SEQUENCE LISTING (194 sequences)

SEQ ID NO: 1 - protein, 742 aa, synthetic construct
  REGION 1..742: note = Synthetic polypeptide
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 2 - DNA, 2464 bp, synthetic construct
  misc_feature 1..2464: note = Synthetic polynucleotide
  source 1..2464: mol_type = other DNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgcgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 3 - DNA, 1045 bp, synthetic construct
  misc_feature 1..1045: note = Synthetic polynucleotide
  source 1..1045: mol_type = other DNA
tcaagcttttggaccctcgtacagaagctaatacgactcactatagggaaataagagagaaaagaagagtaagaagaaatataagagccaccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 4 - DNA, 1072 bp, synthetic construct
  misc_feature 1..1072: note = Synthetic polynucleotide
  source 1..1072: mol_type = other DNA
tcaagcttttggaccctcgtacagaagctaatacgactcactatagggaaataagagagaaaagaagagtaagaagaaatataagagccaccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgcgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 5 - DNA, 2932 bp, synthetic construct
  misc_feature 1..2932: note = Synthetic polynucleotide
  source 1..2932: mol_type = other DNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctgataataggctggagcctcggtggccatgcttcttgccccttgggcctccccccagcccctcctccccttcctgcacccgtacccccgtggtctttgaataaagtctgagtgggcggc

SEQ ID NO: 6 - DNA, 2956 bp, synthetic construct
  misc_feature 1..2956: note = Synthetic polynucleotide
  source 1..2956: mol_type = other DNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtcgattacaaggacgatgacgataagtgataataggctggagcctcggtggccatgcttcttgccccttgggcctccccccagcccctcctccccttcctgcacccgtacccccgtggtctttgaataaagtctgagtgggcggc

SEQ ID NO: 7 - DNA, 2356 bp, synthetic construct
  misc_feature 1..2356: note = Synthetic polynucleotide
  source 1..2356: mol_type = other DNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgactgataataggctggagcctcggtggccatgcttcttgccccttgggcctccccccagcccctcctccccttcctgcacccgtacccccgtggtctttgaataaagtctgagtgggcggc

SEQ ID NO: 8 - DNA, 2383 bp, synthetic construct
  misc_feature 1..2383: note = Synthetic polynucleotide
  source 1..2383: mol_type = other DNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 9 - DNA, 2377 bp, synthetic construct
  misc_feature 1..2377: note = Synthetic polynucleotide
  source 1..2377: mol_type = other DNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgaccaccatcaccaccatcactgatgataataggctggagcctcggtggccatgcttcttgccccttgggcctccccccagcccctcctccccttcctgcacccgtacccccgtggtctttgaataaagtctgagtgggcggc

SEQ ID NO: 10 - DNA, 2287 bp, synthetic construct
  misc_feature 1..2287: note = Synthetic polynucleotide
  source 1..2287: mol_type = other DNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagtgataataggctggagcctcggtggccatgcttcttgccccttgggcctccccccagcccctcctccccttcctgcacccgtacccccgtggtctttgaataaagtctgagtgggcggc

SEQ ID NO: 11 - DNA, 2311 bp, synthetic construct
  misc_feature 1..2311: note = Synthetic polynucleotide
  source 1..2311: mol_type = other DNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcaggattacaaggacgatgacgataagtgataataggctggagcctcggtggccatgcttcttgccccttgggcctccccccagcccctcctccccttcctgcacccgtacccccgtggtctttgaataaagtctgagtgggcggc

SEQ ID NO: 12 - DNA, 2305 bp, synthetic construct
  misc_feature 1..2305: note = Synthetic polynucleotide
  source 1..2305: mol_type = other DNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcaccatcaccaccatcactgataataggctggagcctcggtggccatgcttcttgccccttgggcctccccccagcccctcctccccttcctgcacccgtacccccgtggtctttgaataaagtctgagtgggcggc

SEQ ID NO: 13 - DNA, 724 bp, synthetic construct
  misc_feature 1..724: note = Synthetic polynucleotide
  source 1..724: mol_type = other DNA
tcaagcttttggaccctcgtacagaagctaatacgactcactatagggaaataagagagaaaagaagagtaagaagaaatataagagccaccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagtgataataggctggagcctcggtggccatgcttcttgccccttgggcctccccccagcccctcctccccttcctgcacccgtacccccgtggtctttgaataaagtctgagtgggcggc

SEQ ID NO: 14 - DNA, 748 bp, synthetic construct
  misc_feature 1..748: note = Synthetic polynucleotide
  source 1..748: mol_type = other DNA
tcaagcttttggaccctcgtacagaagctaatacgactcactatagggaaataagagagaaaagaagagtaagaagaaatataagagccaccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcaggattacaaggacgatgacgataagtgataataggctggagcctcggtggccatgcttcttgccccttgggcctccccccagcccctcctccccttcctgcacccgtacccccgtggtctttgaataaagtctgagtgggcggc

SEQ ID NO: 15 - DNA, 853 bp, synthetic construct
  misc_feature 1..853: note = Synthetic polynucleotide
  source 1..853: mol_type = other DNA
tcaagcttttggaccctcgtacagaagctaatacgactcactatagggaaataagagagaaaagaagagtaagaagaaatataagagccaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 16 - DNA, 880 bp, synthetic construct
  misc_feature 1..880: note = Synthetic polynucleotide
  source 1..880: mol_type = other DNA
tcaagcttttggaccctcgtacagaagctaatacgactcactatagggaaataagagagaaaagaagagtaagaagaaatataagagccaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 17 - DNA, 598 bp, synthetic construct
  misc_feature 1..598: note = Synthetic polynucleotide
  source 1..598: mol_type = other DNA
tcaagcttttggaccctcgtacagaagctaatacgactcactatagggaaataagagagaaaagaagagtaagaagaaatataagagccaccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 18 - DNA, 625 bp, synthetic construct
  misc_feature 1..625: note = Synthetic polynucleotide
  source 1..625: mol_type = other DNA
tcaagcttttggaccctcgtacagaagctaatacgactcactatagggaaataagagagaaaagaagagtaagaagaaatataagagccaccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaacgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 19 - DNA, 2434 bp, synthetic construct
  misc_feature 1..2434: note = Synthetic polynucleotide
  source 1..2434: mol_type = other DNA
tcaagcttttggaccctcgtacagaagctaatacgactcactatagggacagacgagagagaagcacgccaattctgcctgcttaagccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgctgataataggctggagcctcggtggccatgcttcttgccccttgggcctccccccagcccctcctccccttcctgcacccgtacccccgtggtctttgaataaagtctgagtgggcggc

SEQ ID NO: 20 - DNA, 1044 bp, synthetic construct
  misc_feature 1..1044: note = Synthetic polynucleotide
  source 1..1044: mol_type = other DNA
tcaagcttttggaccctcgtacagaagctaatacgactcactatagggcttaagcaggcagaattggcccttagcctgtaccagccgaaccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 21 - DNA, 1045 bp, synthetic construct
  misc_feature 1..1045: note = Synthetic polynucleotide
  source 1..1045: mol_type = other DNA
tcaagcttttggaccctcgtacagaagctaatacgactcactatagggtggctcttatatttcttcttactcttcttttctctcttatttccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 22 - DNA, 722 bp, synthetic construct
  misc_feature 1..722: note = Synthetic polynucleotide
  source 1..722: mol_type = other DNA
tcaagcttttggaccctcgtacagaagctaatacgactcactatagggttcggctggtacaggctaaccagaagacagataagagcctccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagtgataataggctggagcctcggtggccatgcttcttgccccttgggcctccccccagcccctcctccccttcctgcacccgtacccccgtggtctttgaataaagtctgagtgggcggc

SEQ ID NO: 23 - DNA, 854 bp, synthetic construct
  misc_feature 1..854: note = Synthetic polynucleotide
  source 1..854: mol_type = other DNA
tcaagcttttggaccctcgtacagaagctaatacgactcactatagggaggctcttatctgtcttctcagtccgaattcgaagtacggctaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 24 - DNA, 853 bp, synthetic construct
  misc_feature 1..853: note = Synthetic polynucleotide
  source 1..853: mol_type = other DNA
tcaagcttttggaccctcgtacagaagctaatacgactcactatagggtggctcttatatttcttcttagtccgaattcgaagtacggctacatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 25 - DNA, 597 bp, synthetic construct
  misc_feature 1..597: note = Synthetic polynucleotide
  source 1..597: mol_type = other DNA
tcaagcttttggaccctcgtacagaagctaatacgactcactatagggtagccgtacttcgaattcggacaagcttctctctcgtctgtccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 26 - DNA, 597 bp, synthetic construct
  misc_feature 1..597: note = Synthetic polynucleotide
  source 1..597: mol_type = other DNA
tcaagcttttggaccctcgtacagaagctaatacgactcactatagggtagccgtacttcgaattcggactttcttttctctcttatttccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 27 - DNA, 3364 bp, synthetic construct
  misc_feature 1..3364: note = Synthetic polynucleotide
  source 1..3364: mol_type = other DNA
tcaagcttttggaccctcgtacagaagctaatacgactcactatagggaaataagagagaaaagaagagtaagaagaaatataagagccaccatggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggcgccatggcgagcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcattgcagaacttgcctcagtgctctcctgatgagattatgtcttatgcccagaaaatctttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctttgagagtctgctctcctagtgtggatgacctacgggccatcgccgaggagtcagatgaggaagaggctattgtagcctacactttggccaccgctggtgccagctcctctgattctctggtgtcacctccagagtcccctgtacccgcgactatccctctgtcctcagtaattgtggctgagaacagtgatcaggaagaaagtgaacagagtgatgaggaacaggaggagggtgctcaggaggagcgggaggacactgtgtctgtcaagtctgagccagtgtctgagatagaggaagttgcctcagaggaagaggaggatggtgctgaggaacccaccgcctctggaggcaagagcacccaccctatggtgactagaagcaaggctgaccagtgataataggctggagcctcggtggccatgcttcttgccccttgggcctccccccagcccctcctccccttcctgcacccgtacccccgtggtctttgaataaagtctgagtgggcggc

SEQ ID NO: 28 - DNA, 3388 bp, synthetic construct
  misc_feature 1..3388: note = Synthetic polynucleotide
  source 1..3388: mol_type = other DNA
tcaagcttttggaccctcgtacagaagctaatacgactcactatagggaaataagagagaaaagaagagtaagaagaaatataagagccaccatggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggcgccatggcgagcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcattgcagaacttgcctcagtgctctcctgatgagattatgtcttatgcccagaaaatctttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctttgagagtctgctctcctagtgtggatgacctacgggccatcgccgaggagtcagatgaggaagaggctattgtagcctacactttggccaccgctggtgccagctcctctgattctctggtgtcacctccagagtcccctgtacccgcgactatccctctgtcctcagtaattgtggctgagaacagtgatcaggaagaaagtgaacagagtgatgaggaacaggaggagggtgctcaggaggagcgggaggacactgtgtctgtcaagtctgagccagtgtctgagatagaggaagttgcctcagaggaagaggaggatggtgctgaggaacccaccgcctctggaggcaagagcacccaccctatggtgactagaagcaaggctgaccaggattacaaggacgatgacgataagtgataataggctggagcctcggtggccatgcttcttgccccttgggcctccccccagcccctcctccccttcctgcacccgtacccccgtggtctttgaataaagtctgagtgggcggc

SEQ ID NO: 29 - DNA, 3352 bp, synthetic construct
  misc_feature 1..3352: note = Synthetic polynucleotide
  source 1..3352: mol_type = other DNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgccgcgccaagaggagcggaagcggagctactaacttcagcctgctgaagcaggctggagacgtggaggagaaccctggacctatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 30 - DNA, 1924 bp, synthetic construct
  misc_feature 1..1924: note = Synthetic polynucleotide
  source 1..1924: mol_type = other DNA
tcaagcttttggaccctcgtacagaagctaatacgactcactatagggaaataagagagaaaagaagagtaagaagaaatataagagccaccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagcgcgccaagaggagcggaagcggagctactaacttcagcctgctgaagcaggctggagacgtggaggagaaccctggacctatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttcgcgccaagaggagcggaagcggagtgaaacagactttgaattttgaccttctcaagttggcgggagacgtggagtccaaccctggacctatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 31 - DNA, 5146 bp, synthetic construct
  misc_feature 1..5146: note = Synthetic polynucleotide
  source 1..5146: mol_type = other DNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgccgcgccaagaggagcggaagcggagctactaacttcagcctgctgaagcaggctggagacgtggaggagaaccctggacctatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgccgcgccaagaggagcggaagcggagtgaaacagactttgaattttgaccttctcaagttggcgggagacgtggagtccaaccctggacctatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagcgcgccaagaggagcggaagcggacagtgtactaattatgctctcttgaaattggctggagatgttgagagcaaccctggacctatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttcgcgccaagaggagcggaagcggagagggcagaggaagtctgctaacatgcggtgacgtcgaggagaatcctggacctatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 32 - protein, 742 aa, synthetic construct; identical to SEQ ID NO: 1
  REGION 1..742: note = Synthetic polypeptide

SEQ ID NO: 33 - protein, 310 aa, synthetic construct
  REGION 1..310: note = Synthetic polypeptide
MPATDTNSTHTTPLHPENQHTLPLHHSTTQPHVQTSDKHADKQHRTQMELDAADYAACAQARQHLYGQTQPQPHAYPNANPQESAHFCTENQHQLTNLLHNIGEGAALGYPVPRAEIRRGGGDWADSASDFDADCWCMWGRFGTMGRQPVVTLLLARQRDGLADWNVVRCRGTGFRAHDSEDGVSVWRQHLVFLLGGHGRRVQLERPSAGEAQARGLLPRIRITPISTSPRPKPPQPTTSTASHPHATARPDHTLFPVPSTPSATVHNPRNYAVQLHAETTRTWRWARRGERGAWMPAETFTCPKDKRPW

SEQ ID NO: 34 - protein, 310 aa, synthetic construct
  REGION 1..310: note = Synthetic polypeptide
MPATDTNSTHTTPLHPEDQHTLPLQHNTTQPHVQTSDKPADKQHRTQMELDAADYAACAQARQHLYGQTQPQLHAYPNANPQESAHFCTDNQHRLTNLLHNIGEGAALGYPVPRAEIRRGGGDWADSASDFDADCWCMWGRFGTMGRQPVVTLLLARQRDGLADWNVVRCRGTGFRAHDSEDGVSVWRQHLVFLLGGHGRRVQLERPSAGEAQARGLLPRIRITPVSTSPRPKAPQPTTSTASHPHATARPDHTLFPVPSTPSATVHNPRNYAVQLHAETTRTWRWARRGERGAWMPAETFTCPKDKRPW

SEQ ID NO: 35 - protein, 466 aa, synthetic construct
  REGION 1..466: note = Synthetic polypeptide
MGRKEMMVRDVPKMVFLISISFLLVSFINCKVMSKALYNRPWRGLVLSKIGKYKLDQLKLEILRQLETTISTKYNVSKQPVKNLTMNMTEFPQYYILAGPIQNYSITYLWFDFYSTQLRKPAKYVYSQYNHTAKTITFRPPPCGTVPSMTCLSEMLNVSKRNDTGEQGCGNFTTFNPMFFNVPRWNTKLYVGPTKVNVDSQTIYFLGLTALLLRYAQRNCTHSFYLVNAMSRNLFRVPKYINGTKLKNTMRKLKRKQAPVKEQFEKKAKKTQSTTTPYFSYTTSAALNVTTNVTYSITTAARRVSTSTIAYRPDSSFMKSIMATQLRDLATWVYTTLRYRQNPFCEPSRNRTAVSEFMKNTHVLIRNETPYTIYGTLDMSSLYYNETMFVENKTASDSNKTTPTSPSMGFQRTFIDPLWDYLDSLLFLDEIRNFSLRSPTYVNLTPPEHRRAVNLSTLNSLWWWLQ

SEQ ID NO: 36 - protein, 135 aa, synthetic construct
  REGION 1..135: note = Synthetic polypeptide
MECNTLVLGLLVLSVVASSNNTSTASTPRPSSSTHASTTVKATTVATTSTTTATSTSSTTSAKPGFTTHDPNVMRPHAHNDFYNAHCTSHMYELSLSSFAAWWTMLNALILMGAFCIVLRHCCFQNFTATTTKGY

SEQ ID NO: 37 - protein, 372 aa, synthetic construct
  REGION 1..372: note = Synthetic polypeptide
MAPSHVDKVNTRTWSASIVFMVLTFVNVSVHLVLSNFPHLGYPCVYYHVVDFERLNMSAYNVMHLHTPMLFLDSVQLVCYAVFMQLVFLAVTIYYLVCWIKISMRKDKGMSLNQSTRDISYMGDSLTAFLFILSMDTFQLFTLTMSFRLPSMIAFMAAVHFFCLTIFNVSMVTQYRSYKRSLFFFSRLHPKLKGTVQFRTLIVNLVEVALGFNTTVVAMALCYGFGNNFFVRTGHMVLAVFVVYAIISIIYFLLIEAVFFQYVKVQFGYHLGAFFGLCGLIYPIVQYDTFLSNEYRTGISWSFGMLFFIWAMFTTCRAVRYFRGRGSGSVKYQALATASGEEVAVLSHHDSLESRRLREEEDDDDDEDFEDA

SEQ ID NO: 38 - protein, 171 aa, synthetic construct
  REGION 1..171: note = Synthetic polypeptide
MSPKDLTPFLTALWLLLGHSRVLRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 39 - protein, 171 aa, synthetic construct; identical to SEQ ID NO: 38
  REGION 1..171: note = Synthetic polypeptide

SEQ ID NO: 40 - protein, 304 aa, synthetic construct
  REGION 1..304: note = Synthetic polypeptide
MPATDTNSTHTTPLHPEHHHSTTQPHAQTSDKHADKQHRTQMELDAADYAACAQARQHLYGQTQPQLHAYPNANPQESAHFCTENQHQLTNLLHNIGEGAALGYPVPRAEIRRGGGDWADSASDFDADCWCMWGRFGTMGRQPVVTLLLARQRDGLADWNVVRCRGTGFRAHDSEDGVSVWRQHLVFLLGGHGRRVQLERPSAGEAQARGLLPRIRITPISTSPRPKPPQPTTSTASHPHATARPDHTLFPVPSTPSATVHNPRNYAVQLHAETTRTWRWARRGERGAWMPAETFTCPKDKRPW

SEQ ID NO: 41 - protein, 310 aa, synthetic construct
  REGION 1..310: note = Synthetic polypeptide
MPATDTNSTHTTPLHPEDQHTLPLHHSTTQPHVQTSDKHADKQHRTQMELDAADYAACAQARQHLYGQTQPQLHAYPNANPQESAHFRTENQHQLTNLLHNIGEGAALGYPVPRAEIRRGGGDWADSASDFDADCWCMWGRFGTMGRQPVVTLLLARQRDGLADWNVVRCRGTGFRAHDSEDGVSVWRQHLVFLLGGHGRRVQLERPSAGEAQARGLLPRIRITPISTSPRPKPPQPTTSTASHPHATARPDHTLFPVPSTPSATVHNPRNYAVQLHAETTRTWRWARRGERGAWMPAETFTCPKDKRPW

SEQ ID NO: 42 - protein, 129 aa, synthetic construct
  REGION 1..129: note = Synthetic polypeptide
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 43 - protein, 129 aa, synthetic construct; identical to SEQ ID NO: 42
  REGION 1..129: note = Synthetic polypeptide

SEQ ID NO: 44 - protein, 310 aa, synthetic construct; identical to SEQ ID NO: 41
  REGION 1..310: note = Synthetic polypeptide

SEQ ID NO: 45 - protein, 310 aa, synthetic construct; identical to SEQ ID NO: 41
  REGION 1..310: note = Synthetic polypeptide

SEQ ID NO: 46 - protein, 310 aa, synthetic construct; identical to SEQ ID NO: 34
  REGION 1..310: note = Synthetic polypeptide

SEQ ID NO: 47 - protein, 310 aa, synthetic construct; identical to SEQ ID NO: 41
  REGION 1..310: note = Synthetic polypeptide

SEQ ID NO: 48 - protein, 760 aa, synthetic construct
  REGION 1..760: note = Synthetic polypeptide
SSFWTLVQKLIRLTIGKERKEEEEIEPPCGQASPPTSSSSPSVSSATYFRHDMAQKPYPNRWTKRFTYCSTPTGDPSASCVKIPPSVPTTAASVTARSSGKTPSVSTFSKAIINTMYSICLDVFLRVLWRSSFTRIPKPWKDTNRDLTLTRWYPKTWPATDLFRSSRHKTAVNSPPLCHRPLTCQYLTFGCHRKPLHTAGQNHIPPQDYTDHTLTRPVSSLMDTIYYSAPSHLVCTKAFTSSTNYVTLKHPRTSSLRCPTTTHPCCLSSAIFHAYFSKRPINATTLYYDKLKNTSSWCLRKINVTLISKTRTFLTPHLTSTTTSAHYYVTAFTVTPWMYSRAVDVRCWTAARKWPSPTHHCSQQPDKKRPAPKSPSHGPTARPHSYKYKNLSPASHKHHHAPRCCCIPRPWTWPNEPFGHRIRSPTSPASYAWSTYSLNRISNISSPNGHYDRSPTLPNYTKRTWPLFFQPSHAKNSTSWAASSTPCWYIRRRDAKSSSKRASVHWPSYHTLRSCLIHTTNTSATCTHPVPVAGDAITRSNASRVSSPMPPSPLPFPPPSPSYLPCNQARWKPSPTCFACRSANPSPRPSPNTSVISQTSTSKVSPTLSPPPSARASSSPRRTVKLNANRATCIPHTASQWRSTFRKTAPFAKAPCNTTTRKASSTSCTCTTRTTSFSPWIPTTKWWSHLRELTTSCFKTVRYKLTSSWTPPTITRTMTISDDNRLEPRWPCFLPLGPPPSPSSPSCTRTPVVFESLSGR

SEQ ID NO: 49 - protein, 759 aa, synthetic construct
  REGION 1..759: note = Synthetic polypeptide
SSFWTLVQKLIRLTIGKERKEEEEIEPPCGQASPPTSSSSPSVSSATYFRHDMAQKPYPNRWTKRFTYCSTPTGDPSASCVKIPPSVPTTAASVTARSSGKTPSVSTFSKAIINTMYSICLDVFLRVLWRSSFTRIPKPWKDTNRDLTLTRWYPKTWPATDLFRSSRHKTAVNSPPLCHRPLTCQYLTFGCHRKPLHTAGQNHIPPQDYTDHTLTRPVSSLMDTIYYSAPSHLVCTKAFTSSTNYVTLKHPRTSSLRCPTTTHPCCLSSAIFHAYFSKRPINATTLYYDKLKNTSSWCLRKINTVTLISKTRTFLTPHLTSTTTSAHYYVTAFTVTPWMYSRAVDVRCWTAARKWPSPTHHCSQQPDKKRPAPKSPSHGPTARPHSYKYKNLSPASHKHHHAPRCCCIPRPWTWPNEPFGHRIRSPTSPASYAWSTYSLNRISNISSPNGHYDRSPTLPNYTKRTWPLFFQPSHAKNSTSWAASSTPCWYIRRRDAKSSSKRASVHWPSYHTLRSCLIHTTNTSATCTHPVPVAGDAITRSNASRVSSPMPPSPLPFPPPSPSYLPCNQARWKPSPTCFACRSANPSPRPSPNTSVISQTSTSKVSPTLSPPPSARASSSPRRTVKLNANRATCIPHTASQWRSTFRKTAPFAKAPCNTTTRKASSTSCTCTTRTTSFSPWIPTTKWWSHLRELTTSCFKTVRYKLTSSWTPPTTITTITDDNRLEPRWPCFLPLGPPPSPSSPSCTRTPVVFESLSGR

SEQ ID NO: 50 - protein, 735 aa, synthetic construct
  REGION 1..735: note = Synthetic polypeptide
SSFWTLVQKLIRLTIGKERKEEEEIEPPWNPGSGAWALTCVSSVWVLRFPHLLLVELLLLTVTIPLIRRLLLTLDPVQSLNALLPKRSAMVLTRPSTTLPSSTEMWWGSIPPSTPIACVLWPRVRILFALNVISSAPRSPSMKTWTRASWWSTNATSSRTPLRYESTRRFRFVVATLTSTPLICWAATRNTWRLLCGRFIISTATVSATVPTAALQARFSWLIIGTAMKTKPCNCPTIIPTPTVPVTRSRINGTAAAAPGSIVRPVIIVWPSLLRAPNILIIFSPLPRVTWLTFLLSTTEPIAMPATLEKTPTSFSFFRTTLSSPTLEDRILRRPTGWWLFLNVRTRSPGIYRTKRMSLVNSLSGKPRNAPFVPKPRTRITFLLPKPPLSYLRSKRTCPTLRWTAYVMRLISYSRFSILHTIKHMKNMETCPSLKPLVVWCSGKVSSKNLWWNSNVWPTAPVILLIIEPKEVQMATMQLIYPTWNRCTIWSTPSCSSPMTRCAVTSTGRWRKSQKPGVWINGAPRSSRNSARSTRQPFSRPFTTNRLPRVSWVMSWAWPAAPSTKPASRCCVITRSRQDAATHDPWSSLISPTARTCSTVNWARTTKSCWATTALRNVSFPASRSSSPGTRPTSTWTTSSNALTSAVSPPSTASPWISTRWKIPTSGYWNFTRRKSCVPATFLTSKRSCANSTRTSSDNRLEPRWPCFLPLGPPPSPSSPSCTRTPVVFESLSGR

SEQ ID NO: 51 - protein, 744 aa, synthetic construct
  REGION 1..744: note = Synthetic polypeptide
SSFWTLVQKLIRLTIGKERKEEEEIEPPWNPGSGAWSALTCVSSVWVLRFPHLLLVELLLLTVTIPLIRRLLLTLDPVQSLNALLPKRSAMVLTRPSTTLPSSTEMWWGSIPPSTPIACVLWPRVRILFALNVISSAPRSPSMKTWTRASWWSTNATSSRTPLRYESTRRFRFVVATLTSTPLICWAATRNTWRLLCGRFIISTATVSATVPTAALQARFSWLIIGTAMKTKPCNCPTIIPTPTVPVTRSRINGTAAAAPGSIVRPVIIVWPSLLRAPNILIIFSPLPRVTWLTFLLSTTEPIAMPATLEKTPTSFSFFRTTLSSPTLEDRILRRPTGWWLFLNVRTRSPGIYRTKRMSLVNSLSGKPRNAPFVPKPRTRITFLLPKPPLSYLRSKRTCPTLRWTAYVMRLISYSRFSILHTIKHMKNMETCPSLKPLVVWCSGKVSSKNLWWNSNVWPTAPVILLIIEPKEVQMATMQLIYPTWNRCTIWSTPSCSSPMTRCAVTSTGRWRKSQKPGVWINGAPRSSRNSARSTRQPFSRPFTTNRLPRVSWVMSWAWPAAPSTKPASRCCVITRSRQDAATHDPWSSLISPTARTCSTVNWARTTKSCWATTALRNVSFPASRSSSPGTRPTSTWTTSSNALTSAVSPPSTASPWISTRWKIPTSGYWNFTRRKSCVPATFLTSKRSCANSTRTSRITRTMTISDNRLEPRWPCFLPLGPPPSPSSPSCTRTPVVFESLSGR

SEQ ID NO: 52 - protein, 742 aa, synthetic construct
  REGION 1..742: note = Synthetic polypeptide
SSFWTLVQKLIRLTIGKERKEEEEIEPPWNPGSGAWSALTCVSSVWVLRFPHLLLVELLLLTVTIPLIRRLLLTLDPVQSLNALLPKRSAMVLTRPSTTLPSSTEMWWGSIPPSTPIACVLWPRVRILFALNVISSAPRSPSMKTWTRASWWSTNATSSRTPLRYESTRRFRFVVATLTSTPLICWAATRNTWRLLCGRFIISTATVSATVPTAALQARFSWLIIGTAMKTKPCNCPTIIPTPTVPVTRSRINGTAAAAPGSIVRPVIIVWPSLLRAPNILIIFSPLPRVTWLTFLLSTTEPIAMPATLEKTPTSFSFFRTTLSSPTLEDRILRRPTGWWLFLNVRTRSPGIYRTKRMSLVNSLSGKPRNAPFVPKPRTRITFLLPKPPLSYLRSKRTCPTLRWTAYVMRLISYSRFSILHTIKHMKNMETCPSLKPLVVWCSGKVSSKNLWWNSNVWPTAPVILLIIEPKEVQMATMQLIYPTWNRCTIWSTPSCSSPMTRCAVTSTGRWRKSQKPGVWINGAPRSSRNSARSTRQPFSRPFTTNRLPRVSWVMSWAWPAAPSTKPASRCCVITRSRQDAATHDPWSSLISPTARTCSTVNWARTTKSCWATTALRNVSFPASRSSSPGTRPTSTWTTSSNALTSAVSPPSTASPWISTRWKIPTSGYWNFTRRKSCVPATFLTSKRSCANSTRTSSTITTITDNRLEPRWPCFLPLGPPPSPSSPSCTRTPVVFESLSGR

SEQ ID NO: 53 - protein, 18 aa, synthetic construct
  REGION 1..18: note = Synthetic polypeptide
MDWTWILFLVAAATRVHS

SEQ ID NO: 54 - protein, 20 aa, synthetic construct
  REGION 1..20: note = Synthetic polypeptide
METPAQLLFLLLLWLPDTTG

SEQ ID NO: 55 - protein, 278 aa, synthetic construct
  REGION 1..278: note = Synthetic polypeptide
MCRRPDCGFSFSPGPVILLWCCLLLPIVSSAAVSVAPTAAEKVPAECPELTRRCLLGEVFEGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 56 - protein, 907 aa, synthetic construct
  REGION 1..907: note = Synthetic polypeptide
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 57 - protein, 214 aa, synthetic construct
  REGION 1..214: note = Synthetic polypeptide
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 58 - DNA, 2437 bp, synthetic construct
  misc_feature 1..2437: note = Synthetic polynucleotide
  source 1..2437: mol_type = other DNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaacttcttccaaagctataatcaatactatgtattccatatgcctcgatgtctctttgcgggtcctctggcggagcagtttctgaaccaggtagatctgaccgaaaccctggaaagataccaacagagacttaacacttacgcgctggtatccaaagacctggccagctaccgatctttctcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgagaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaagaacggtacggtactagaagtaactgacgtcgtcgtggacgccaccgacagtcgtctcctcatgatgtccgtctacgcgctatcggccatcatcggcatctatctgctctaccgcatgctcaagacatgctgataataggctggagcctcggtggccatgcttcttgccccttgggcctccccccagcccctcctccccttcctgcacccgtacccccgtggtctttgaataaagtctgagtgggcggc

SEQ ID NO: 59 - protein, 742 aa, synthetic construct; identical to SEQ ID NO: 1
  REGION 1..742: note = Synthetic polypeptide

SEQ ID NO: 60 - DNA, 1045 bp, synthetic construct; identical to SEQ ID NO: 3
  misc_feature 1..1045: note = Synthetic polynucleotide
  source 1..1045: mol_type = other DNA

SEQ ID NO: 61 - protein, 278 aa, synthetic construct; identical to SEQ ID NO: 55
  REGION 1..278: note = Synthetic polypeptide

SEQ ID NO: 62 - DNA, 724 bp, synthetic construct; identical to SEQ ID NO: 13
  misc_feature 1..724: note = Synthetic polynucleotide
  source 1..724: mol_type = other DNA

SEQ ID NO: 63 - protein, 171 aa, synthetic construct
  REGION 1..171: note = Synthetic polypeptide
MSPKDLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 64 - DNA, 846 bp, synthetic construct
  misc_feature 1..846: note = Synthetic polynucleotide
  source 1..846: mol_type = other DNA
tcaagcttttggaccctcgtacagaagctaatacgactcactatagggaaataagagagaaaagaagagtaagaagaaatataagagccaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcgctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 65 - protein, 214 aa, synthetic construct; identical to SEQ ID NO: 57
  REGION 1..214: note = Synthetic polypeptide

SEQ ID NO: 66 - DNA, 598 bp, synthetic construct
  misc_feature 1..598: note = Synthetic polynucleotide
  source 1..598: mol_type = other DNA
tcaagcttttggaccctcgtacagaagctaatacgactcactatagggaaataagagagaaaagaagagtaagaagaaatataagagccaccatgcggctgtgtcgggtgtggctgtctgtttgtctgtgcgccgtggtgctgggtcagtgccagcgggaaaccgcggaaaagaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 67 - protein, 129 aa, synthetic construct; identical to SEQ ID NO: 42
  REGION 1..129: note = Synthetic polypeptide

SEQ ID NO: 68 - DNA, 2933 bp, synthetic construct
  misc_feature 1..2933: note = Synthetic polynucleotide
  source 1..2933: mol_type = other DNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtcttgataataggctggagcctcggtggccatgcttcttgccccttgggcctccccccagcccctcctccccttcctgcacccgtacccccgtggtctttgaataaagtctgagtgggcggc

SEQ ID NO: 69 - protein, 907 aa, synthetic construct; identical to SEQ ID NO: 56
  REGION 1..907: note = Synthetic polypeptide

SEQ ID NO: 70 - DNA, 1882 bp, synthetic construct
  misc_feature 1..1882: note = Synthetic polynucleotide
  source 1..1882: mol_type = other DNA
tcaagcttttggaccctcgtacagaagctaatacgactcactatagggaaataagagagaaaagaagagtaagaagaaatataagagccaccatggagtcgcgcggtcgccgttgtcccgaaatgatatccgtactgggtcccatttcggggcacgtgctgaaagccgtgtttagtcgcggcgatacgccggtgctgccgcacgagacgagactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggagccatggcgagcgcctccacttccgcgggctcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttgataataggctggagcctcggtggccatgcttcttgccccttgggcctccccccagcccctcctccccttcctgcacccgtacccccgtggtctttgaataaagtctgagtgggcggc

SEQ ID NO: 71 - protein, 557 aa, synthetic construct
  REGION 1..557: note = Synthetic polypeptide
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 72 - DNA, 3364 bp, synthetic construct; identical to SEQ ID NO: 27
  misc_feature 1..3364: note = Synthetic polynucleotide
  source 1..3364: mol_type = other DNA

SEQ ID NO: 73 - protein, 1051 aa, synthetic construct
  REGION 1..1051: note = Synthetic polypeptide
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRGESSAKRKMDPDNPDEGPSSKVPRPETPVTKATTFLQTMLRKEVNSQLSLGDPLFPELAEESLKTFEQVTEDCNENPEKDVLTELVKQIKVRVDMVRHRIKEHMLKKYTQTEEKFTGAFNMMGGCLQNALDILDKVHEPFEDMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMSYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGASSSDSLVSPPESPVPATIPLSSVIVAENSDQEESEQSDEEQEEGAQEEREDTVSVKSEPVSEIEEVASEEEEDGAEEPTASGGKSTHPMVTRSKADQ

SEQ ID NO: 74 - DNA, 3153 bp, synthetic construct
  misc_feature 1..3153: note = Synthetic polynucleotide
  source 1..3153: mol_type = other DNA
atggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggcgccatggcgagcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcattgcagaacttgcctcagtgctctcctgatgagattatgtcttatgcccagaaaatctttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctttgagagtctgctctcctagtgtggatgacctacgggccatcgccgaggagtcagatgaggaagaggctattgtagcctacactttggccaccgctggtgccagctcctctgattctctggtgtcacctccagagtcccctgtacccgcgactatccctctgtcctcagtaattgtggctgagaacagtgatcaggaagaaagtgaacagagtgatgaggaacaggaggagggtgctcaggaggagcgggaggacactgtgtctgtcaagtctgagccagtgtctgagatagaggaagttgcctcagaggaagaggaggatggtgctgaggaacccaccgcctctggaggcaagagcacccaccctatggtgactagaagcaaggctgaccag

SEQ ID NO: 75 - DNA, 3424 bp, synthetic construct
  misc_feature 1..3424: note = Synthetic polynucleotide
  source 1..3424: mol_type = other DNA
gggaaataagagagaaaagaagagtaagaagaaatataagagccaccatggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggcgccatggcgagcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcattgcagaacttgcctcagtgctctcctgatgagattatgtcttatgcccagaaaatctttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctttgagagtctgctctcctagtgtggatgacctacgggccatcgccgaggagtcagatgaggaagaggctattgtagcctacactttggccaccgctggtgccagctcctctgattctctggtgtcacctccagagtcccctgtacccgcgactatccctctgtcctcagtaattgtggctgagaacagtgatcaggaagaaagtgaacagagtgatgaggaacaggaggagggtgctcaggaggagcgggaggacactgtgtctgtcaagtctgagccagtgtctgagatagaggaagttgcctcagaggaagaggaggatggtgctgaggaacccaccgcctctggaggcaagagcacccaccctatggtgactagaagcaaggctgaccagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 76 - DNA, 3352 bp, synthetic construct
  misc_feature 1..3352: note = Synthetic polynucleotide
  source 1..3352: mol_type = other DNA
tcaagcttttggaccctcgtacagaagctaatacgactcactatagggaaataagagagaaaagaagagtaagaagaaatataagagccaccatggagtcgcgcggtcgccgttgtcccgaaatgatatccgtactgggtcccatttcgggtcatgtgctgaaagcggtgtttagtcgcggcgatacgccagtactgccgcacgagacgcgactcctgcagacaggtatccacgtacgcgtgagccagccctcgctcatcctggtgtcgcagtacacgcccgactcgacgccatgccaccgcggcgacaatcagctgcaggtgcagcacacgtactttacgggcagcgaggtggagaacgtgtcggtcaacgtccacaaccccacgggtcgaagcatctgcccctctcaagagcccatgtcgatctatgtgtacgcgctgccgctcaagatgctgaacatcccgagcatcaacgtgcaccactacccgagcgcggccgagcgcaaacaccgacacctgcccgtagccgacgctgttattcacgcgtcgggcaagcagatgtggcaagcgcgcctcacggtctcgggactagcctggacgcgtcagcagaaccagtggaaagagcccgacgtctactacacgtcagcgttcgtgtttcccaccaaggacgtggcactgcgccacgtggtgtgtgcgcacgagctggtttgctccatggagaatacgcgcgcaaccaagatgcaggtgataggtgatcaatacgtcaaggtgtacctggagtccttctgcgaggatgtgccctccggtaagctctttatgcacgtcacgctgggctctgacgtggaagaggacctaacgatgacccgcaatccgcaacccttcatgcgcccccacgagcgcaacggctttacggtgttgtgtcctaaaaatatgataatcaaaccaggcaagatctcgcacatcatgctggatgtggcttttacctcacacgagcattttgggctgctgtgtcccaagagcatcccgggcctgagcatctcaggtaacctgttgatgaacgggcagcaaatctttctcgaggtgcaagctatacgcgagaccgtcgaactgcgtcagtacgatcccgtggctgcgctgttctttttcgatatcgacttgttgctgcagcgcgggcctcagtacagcgagcaccccaccttcaccagccagtatcgcatccagggcaagcttgagtaccgacacacctgggaccggcacgacgagggagccgcccagggcgacgacgacgtctggacctctggatcggactccgacgaagaactcgtaacgaccgagcgtaagaccccccgcgtcaccggcggcggcgccatggcgtccgcctcaacttccgcgggctcagcatcctcggctacggcgtgcacggcgggcgttatgacacgtggcagacttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgctcgcaggccatggcggcattgcagaacttgcctcagtgctctcctgatgagattatgtcttatgcccagaaaatctttaagattttggatgaggagcgagacaaggtgcttacgcacattgatcacatatttatggatatcctcactacatgtgttgaaacgatgtgcaatgagtacaaggtcactagtgacgcttgtatgatgaccatgtacgggggcatatctctcttaagtgaattctgtcgggtgctgtgctgctacgtcttagaggagactagtgtgatgctggccaagcggcctctgataaccaagcctgaggtcatcagtgtaatgaagcgccgcattgaggagatctgcatgaaggtctttgcccagtacattctgggggccgatcctttgagagtctgctctccaagtgtggatgacctacgggccatcgccgaggagtcagacgaggaagaggctattgtagcctacactttggccaccgctggtgccagctcctctgactctctggtgtcacctccagaatcccctgtgcccgcgacaatccctctgtcctcagtaattgtggctgagaacagtgatcaggaagaaagtgaacagagtgatgaggaacaggaggagggtgctcaggaggagcgggaggatactgtgtctgtcaagtctgagccagtgtctgaaattgaggaagttgcctcagaggaagaggaggatggtgctgaggaacccaccgcctctggaggcaagtccacccaccctatggtaactagatcaaaggctgaccagtgataataggctggagcctcggtggccatgcttcttgccccttgggcctccccccagcccctcctccccttcctgcacccgtacccccgtggtctttgaataaagtctgagtgggcggc

SEQ ID NO: 77 - protein, 1047 aa, synthetic construct
  REGION 1..1047: note = Synthetic polypeptide
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRGESSAKRKMDPDNPDEGPSSKVPRPETPVTKATTFLQTMLRKEVNSQLSLGDPLFPELAEESLKTFEQVTEDCNENPEKDVLTELVKQIKVRVDMVRHRIKEHMLKKYTQTEEKFTGAFNMMGGCLQNALDILDKVHEPFEDMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMSYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGASSSDSLVSPPESPVPATIPLSSVIVAENSDQEESEQSDEEQEEGAQEEREDTVSVKSEPVSEIEEVASEEEEDGAEEPTASGGKSTHPMVTRSKADQ

SEQ ID NO: 78 - DNA, 3141 bp, synthetic construct
  misc_feature 1..3141: note = Synthetic polynucleotide
  source 1..3141: mol_type = other DNA
atggagtcgcgcggtcgccgttgtcccgaaatgatatccgtactgggtcccatttcgggtcatgtgctgaaagcggtgtttagtcgcggcgatacgccagtactgccgcacgagacgcgactcctgcagacaggtatccacgtacgcgtgagccagccctcgctcatcctggtgtcgcagtacacgcccgactcgacgccatgccaccgcggcgacaatcagctgcaggtgcagcacacgtactttacgggcagcgaggtggagaacgtgtcggtcaacgtccacaaccccacgggtcgaagcatctgcccctctcaagagcccatgtcgatctatgtgtacgcgctgccgctcaagatgctgaacatcccgagcatcaacgtgcaccactacccgagcgcggccgagcgcaaacaccgacacctgcccgtagccgacgctgttattcacgcgtcgggcaagcagatgtggcaagcgcgcctcacggtctcgggactagcctggacgcgtcagcagaaccagtggaaagagcccgacgtctactacacgtcagcgttcgtgtttcccaccaaggacgtggcactgcgccacgtggtgtgtgcgcacgagctggtttgctccatggagaatacgcgcgcaaccaagatgcaggtgataggtgatcaatacgtcaaggtgtacctggagtccttctgcgaggatgtgccctccggtaagctctttatgcacgtcacgctgggctctgacgtggaagaggacctaacgatgacccgcaatccgcaacccttcatgcgcccccacgagcgcaacggctttacggtgttgtgtcctaaaaatatgataatcaaaccaggcaagatctcgcacatcatgctggatgtggcttttacctcacacgagcattttgggctgctgtgtcccaagagcatcccgggcctgagcatctcaggtaacctgttgatgaacgggcagcaaatctttctcgaggtgcaagctatacgcgagaccgtcgaactgcgtcagtacgatcccgtggctgcgctgttctttttcgatatcgacttgttgctgcagcgcgggcctcagtacagcgagcaccccaccttcaccagccagtatcgcatccagggcaagcttgagtaccgacacacctgggaccggcacgacgagggagccgcccagggcgacgacgacgtctggacctctggatcggactccgacgaagaactcgtaacgaccgagcgtaagaccccccgcgtcaccggcggcggcgccatggcgtccgcctcaacttccgcgggctcagcatcctcggctacggcgtgcacggcgggcgttatgacacgtggcagacttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgctcgcaggccatggcggcattgcagaacttgcctcagtgctctcctgatgagattatgtcttatgcccagaaaatctttaagattttggatgaggagcgagacaaggtgcttacgcacattgatcacatatttatggatatcctcactacatgtgttgaaacgatgtgcaatgagtacaaggtcactagtgacgcttgtatgatgaccatgtacgggggcatatctctcttaagtgaattctgtcgggtgctgtgctgctacgtcttagaggagactagtgtgatgctggccaagcggcctctgataaccaagcctgaggtcatcagtgtaatgaagcgccgcattgaggagatctgcatgaaggtctttgcccagtacattctgggggccgatcctttgagagtctgctctccaagtgtggatgacctacgggccatcgccgaggagtcagacgaggaagaggctattgtagcctacactttggccaccgctggtgccagctcctctgactctctggtgtcacctccagaatcccctgtgcccgcgacaatccctctgtcctcagtaattgtggctgagaacagtgatcaggaagaaagtgaacagagtgatgaggaacaggaggagggtgctcaggaggagcgggaggatactgtgtctgtcaagtctgagccagtgtctgaaattgaggaagttgcctcagaggaagaggaggatggtgctgaggaacccaccgcctctggaggcaagtccacccaccctatggtaactagatcaaaggctgaccag

SEQ ID NO: 79 - DNA, 3412 bp, synthetic construct
  misc_feature 1..3412: note = Synthetic polynucleotide
  source 1..3412: mol_type = other DNA
gggaaataagagagaaaagaagagtaagaagaaatataagagccaccatggagtcgcgcggtcgccgttgtcccgaaatgatatccgtactgggtcccatttcgggtcatgtgctgaaagcggtgtttagtcgcggcgatacgccagtactgccgcacgagacgcgactcctgcagacaggtatccacgtacgcgtgagccagccctcgctcatcctggtgtcgcagtacacgcccgactcgacgccatgccaccgcggcgacaatcagctgcaggtgcagcacacgtactttacgggcagcgaggtggagaacgtgtcggtcaacgtccacaaccccacgggtcgaagcatctgcccctctcaagagcccatgtcgatctatgtgtacgcgctgccgctcaagatgctgaacatcccgagcatcaacgtgcaccactacccgagcgcggccgagcgcaaacaccgacacctgcccgtagccgacgctgttattcacgcgtcgggcaagcagatgtggcaagcgcgcctcacggtctcgggactagcctggacgcgtcagcagaaccagtggaaagagcccgacgtctactacacgtcagcgttcgtgtttcccaccaaggacgtggcactgcgccacgtggtgtgtgcgcacgagctggtttgctccatggagaatacgcgcgcaaccaagatgcaggtgataggtgatcaatacgtcaaggtgtacctggagtccttctgcgaggatgtgccctccggtaagctctttatgcacgtcacgctgggctctgacgtggaagaggacctaacgatgacccgcaatccgcaacccttcatgcgcccccacgagcgcaacggctttacggtgttgtgtcctaaaaatatgataatcaaaccaggcaagatctcgcacatcatgctggatgtggcttttacctcacacgagcattttgggctgctgtgtcccaagagcatcccgggcctgagcatctcaggtaacctgttgatgaacgggcagcaaatctttctcgaggtgcaagctatacgcgagaccgtcgaactgcgtcagtacgatcccgtggctgcgctgttctttttcgatatcgacttgttgctgcagcgcgggcctcagtacagcgagcaccccaccttcaccagccagtatcgcatccagggcaagcttgagtaccgacacacctgggaccggcacgacgagggagccgcccagggcgacgacgacgtctggacctctggatcggactccgacgaagaactcgtaacgaccgagcgtaagaccccccgcgtcaccggcggcggcgccatggcgtccgcctcaacttccgcgggctcagcatcctcggctacggcgtgcacggcgggcgttatgacacgtggcagacttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgctcgcaggccatggcggcattgcagaacttgcctcagtgctctcctgatgagattatgtcttatgcccagaaaatctttaagattttggatgaggagcgagacaaggtgcttacgcacattgatcacatatttatggatatcctcactacatgtgttgaaacgatgtgcaatgagtacaaggtcactagtgacgcttgtatgatgaccatgtacgggggcatatctctcttaagtgaattctgtcgggtgctgtgctgctacgtcttagaggagactagtgtgatgctggccaagcggcctctgataaccaagcctgaggtcatcagtgtaatgaagcgccgcattgaggagatctgcatgaaggtctttgcccagtacattctgggggccgatcctttgagagtctgctctccaagtgtggatgacctacgggccatcgccgaggagtcagacgaggaagaggctattgtagcctacactttggccaccgctggtgccagctcctctgactctctggtgtcacctccagaatcccctgtgcccgcgacaatccctctgtcctcagtaattgtggctgagaacagtgatcaggaagaaagtgaacagagtgatgaggaacaggaggagggtgctcaggaggagcgggaggatactgtgtctgtcaagtctgagccagtgtctgaaattgaggaagttgcctcagaggaagaggaggatggtgctgaggaacccaccgcctctggaggcaagtccacccaccctatggtaactagatcaaaggctgaccagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 80 - protein, 491 aa, synthetic construct
  REGION 1..491: note = Synthetic polypeptide
MESSAKRKMDPDNPDEGPSSKVPRPETPVTKATTFLQTMLRKEVNSQLSLGDPLFPELAEESLKTFEQVTEDCNENPEKDVLTELVKQIKVRVDMVRHRIKEHMLKKYTQTEEKFTGAFNMMGGCLQNALDILDKVHEPFEDMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMSYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGASSSDSLVSPPESPVPATIPLSSVIVAENSDQEESEQSDEEQEEGAQEEREDTVSVKSEPVSEIEEVASEEEEDGAEEPTASGGKSTHPMVTRSKADQ

SEQ ID NO: 81 - protein, 580 aa, synthetic construct
  REGION 1..580: note = Synthetic polypeptide
MESSAKRKMDPDNPDEGPSSKVPRPETPVTKATTFLQTMLRKEVNSQLSLGDPLFPELAEESLKTFEQVTEDCNENPEKDVLTELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRHTNRSLEYKNLPFMIPSMHQVLEEAIKVCKTMQVNNKGIQIIYTRNHEVKNEVDQVRCRLGSMCNLALSTPFLMEHTMPVTHPPDVAQRTADACNDGVKAVWNLKELHTHQLCPRSSDYRNMIIHAATPVDLLGALNLCLPLMQKFPKQVMVRIFSTNQGGFMLPIYETAAKAYAVGQFEKPTETPPEDLDTLSLAIEAAIQDLRNKSQ

SEQ ID NO: 82 - protein, 561 aa, synthetic construct
  REGION 1..561: note = Synthetic polypeptide
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 83 - protein, 698 aa, synthetic construct
  REGION 1..698: note = Synthetic polypeptide
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQHHHHHH

SEQ ID NO: 84 - DNA, 1473 bp, synthetic construct
  misc_feature 1..1473: note = Synthetic polynucleotide
  source 1..1473: mol_type = other DNA
atggagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaagacctttgaacaagtgaccgaggattgcaacgagaaccccgagaaagatgtcctgacagaactcgtcaaacagattaaggttcgagtggacatggtgcggcatagaatcaaggagcacatgctgaagaaatatacccagacggaagagaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttgggcggtgcactgcaggctaaggcccgtgctaagaaggatgaacttaggagaaagatgatgtatatgtgctacaggaatatagagttctttaccaagaactcagccttccctaagaccaccaatggctgcagtcaggccatggcggcattgcagaacttgcctcagtgctctcctgatgagattatgtcttatgcccagaagatctttaagattttggatgaggagagagacaaggtgctcacgcacattgatcacatatttatggatatcctcactacatgtgtggaaacaatgtgtaatgagtacaaggtcactagtgacgcttgtatgatgaccatgtacggcggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggagccgatcctttgagagtctgctctcctagtgtggatgacctacgggccatcgccgaggagtcagatgaggaagaggctattgtagcctacactttggccaccgctggtgccagctcctctgattctctggtgtcacctccagagtcccctgtacccgcgactatccctctgtcctcagtaattgtggctgagaacagtgatcaggaagaaagtgaacagagtgatgaggaacaggaggagggtgctcaggaggagcgggaggacactgtgtctgtcaagtctgagccagtgtctgagatagaggaagttgcctcagaggaagaggaggatggtgctgaggaacccaccgcctctggaggcaagagcacccaccctatggtgactagaagcaaggctgaccag

SEQ ID NO: 85 - DNA, 1740 bp, synthetic construct
  misc_feature 1..1740: note = Synthetic polynucleotide
  source 1..1740: mol_type = other DNA
atggagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaagacctttgaacaagtgaccgaggattgcaacgagaaccccgagaaagatgtcctgacagaactcggtgacatcctcgcccaggctgtcaatcatgccggtatcgattccagtagcaccggccccacgctgacaacccactcttgcagcgttagcagcgcccctcttaacaagccgacgcccaccagcgtcgcggttactaacactcctctccccggggcatccgctactcccgagctcagcccgcgtaagaaaccgcgcaagaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgagacccgcggtgctaccgcgtcttccccttccaccggcagcggcacgccgcgagtgacctctcccacgcacccgctctcccagatgaaccaccctcctcttcccgatcctttgggccggcccgatgaagatagttcctcttcgtcttcctcctcctgcagttcggcttcggacagcgagagtgagtccgaggagatgaaatgcagcagtggcggaggagcatccgtgacctcgagccaccatgggcgcggcggttttggtggcgcggcctcctcctctctgctgagctgcggacatcagagcagcggcggggcgagcaccggacctcgcaagaagaagagcaaacgcatctccgagttggacaacgagaaggtgcgcaatatcatgaaagataagaacacgcccttctgcacacccaacgtgcagactcggcggggtcgcgtcaagattgacgaggtgagccgcatgttccgtcacaccaatcgttctcttgagtacaagaatctgccattcatgatccctagtatgcaccaagtgttagaagaggccatcaaagtttgcaagaccatgcaggtgaacaacaagggcattcagatcatctacacccgcaatcatgaagtgaagaatgaggtggatcaggtacggtgtcgcctgggtagcatgtgcaacctggccctctccactcccttcctcatggagcacactatgcctgtgacacaccctcctgatgtggcgcagcgcacggccgatgcttgtaacgacggtgtcaaggccgtgtggaacctcaaagaactgcacacccaccaattgtgcccgcgctcttctgattaccgcaacatgattatccacgctgccacgcccgtggacctgttgggcgctctcaacctgtgcctgcccctgatgcagaagtttcccaaacaggtcatggtgcgcatcttctccaccaaccagggtgggttcatgctgcctatctacgagacggccgcgaaggcctacgccgtggggcagtttgagaagcccaccgagacccctcccgaagacctggacaccctgagcctggccatcgaggcagccatccaggacctgaggaacaaatctcag

SEQ ID NO: 86 - DNA, 2721 bp, synthetic construct
  misc_feature 1..2721: note = Synthetic polynucleotide
  source 1..2721: mol_type = other DNA
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtc

SEQ ID NO: 87 - DNA, 2226 bp, synthetic construct
  misc_feature 1..2226: note = Synthetic polynucleotide
  source 1..2226: mol_type = other DNA
atgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaacttcttccaaagctataatcaatactatgtattccatatgcctcgatgtctctttgcgggtcctctggcggagcagtttctgaaccaggtagatctgaccgaaaccctggaaagataccaacagagacttaacacttacgcgctggtatccaaagacctggccagctaccgatctttctcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgagaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaagaacggtacggtactagaagtaactgacgtcgtcgtggacgccaccgacagtcgtctcctcatgatgtccgtctacgcgctatcggccatcatcggcatctatctgctctaccgcatgctcaagacatgc

SEQ ID NO: 88 - DNA, 2226 bp, synthetic construct; identical to SEQ ID NO: 87
  misc_feature 1..2226: note = Synthetic polynucleotide
  source 1..2226: mol_type = other DNA

SEQ ID NO: 89 - DNA, 513 bp, synthetic construct
  misc_feature 1..513: note = Synthetic polynucleotide
  source 1..513: mol_type = other DNA
atgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcag

SEQ ID NO: 90 - DNA, 834 bp, synthetic construct
  misc_feature 1..834: note = Synthetic polynucleotide
  source 1..834: mol_type = other DNA
atgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgc

SEQ ID NO: 91 - DNA, 642 bp, synthetic construct
  misc_feature 1..642: note = Synthetic polynucleotide
  source 1..642: mol_type = other DNA
atgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtt

SEQ ID NO: 92 - DNA, 1671 bp, synthetic construct
  misc_feature 1..1671: note = Synthetic polynucleotide
  source 1..1671: mol_type = other DNA
atggagtcgcgcggtcgccgttgtcccgaaatgatatccgtactgggtcccatttcggggcacgtgctgaaagccgtgtttagtcgcggcgatacgccggtgctgccgcacgagacgagactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggagccatggcgagcgcctccacttccgcgggctcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggt

SEQ ID NO: 93 - DNA, 1683 bp, synthetic construct
  misc_feature 1..1683: note = Synthetic polynucleotide
  source 1..1683: mol_type = other DNA
atggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaagaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggcgccatggcgagcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaagaagcaccgaggt

SEQ ID NO: 94 - DNA, 2721 bp, synthetic construct
  misc_feature 1..2721: note = Synthetic polynucleotide
  source 1..2721: mol_type = other DNA
atggaaagccggatctggtgtcttgtggtgtgcgtgaatctttgcatcgtgtgcttgggtgccgccgtgtcatctagcagcacaagaggcacctccgccactcactcacaccacagcagccacacgaccagcgccgctcactccagaagcggctctgtaagccagagagtgaccagttctcaaaccgtcagccacggcgtcaatgagacgatatataatacaaccctgaagtatggagacgtggtgggtgtcaataccaccaagtacccttatcgcgtgtgcagcatggcccagggcactgacctgatcagattcgagagaaatatcgtctgcacctccatgaagcctatcaacgaggaccttgacgagggcatcatggttgtctacaagagaaacattgtggctcacaccttcaaggtgagagtgtatcagaaagtactgacctttaggagatcctacgcttacatccacaccacgtacctgctcggctccaacaccgagtatgtggctccacccatgtgggagattcatcacatcaattcccacagccaatgttacagttcctatagcagagtcattgctggtaccgtgttcgtcgcttaccacagagacagctatgagaacaagaccatgcagttgatgcccgatgactactccaatacacactctacaaggtatgtgacagtcaaagatcagtggcacagccggggcagcacctggctgtaccgagagacatgtaatctgaattgtatggtgactatcactacagccaggagcaaatatccataccacttcttcgccactagcaccggggacgtcgtggacatttccccattctacaatggcacaaacagaaacgccagctacttcggcgagaatgccgacaagttctttatattccccaactataccatcgtgagcgacttcggccgccccaacagcgccctggaaacccaccggctcgtggcctttctcgagcgggccgatagcgtcatatcctgggacatccaggacgagaagaatgtgacatgccagctgaccttctgggaggcctccgagcgtaccatccggtccgaggcagaggacagctaccatttcagcagcgccaagatgaccgcaaccttcctcagtaagaaacaggaggttaacatgtctgattctgccctggactgcgtgcgcgatgaggcaatcaacaagctgcagcagatcttcaacacatcttacaaccaaacttacgagaagtacggcaacgtcagcgtgttcgagacaacaggaggcctggtagtgttctggcaaggtatcaagcagaagagtctggtggagctcgagcgactggctaaccgcagctccctcaacctgacccataataggacaaagagaagcaccgacggcaacaacgctactcatttgagcaacatggaatccgtgcacaacctggtgtatgcccagctgcagttcacttacgacaccctgagaggctacatcaatagagccttagctcagatcgcagaggcttggtgtgtggaccagcgaagaactctggaggtgttcaaggagttaagtaagatcaatccatccgccatcctgtctgctatctacaacaagcccattgccgccaggttcatgggagatgtgctcggcctggctagttgtgtcaccatcaaccagacctccgtgaaggtgctgcgggacatgaatgtgaaggagagccccggtcggtgttactccagaccagtggtgattttcaacttcgccaacagctcctatgtgcagtacggacagctcggagaggataacgagatcttgctcggcaatcacagaactgaggagtgtcagctgccatcactgaagatatttattgccgggaattccgcctacgaatacgttgactaccttttcaagagaatgatcgacctgagcagcatcagcaccgtcgacagcatgattgctctcgatatcgaccctctggagaacaccgactttagagtccttgagctgtattcacagaaggagctgaggagctccaatgtgttcgacctggaggaaatcatgagagagttcaactcttacaagcagcgggtgaagtacgtggaggataaggtagtggacccactcccaccatacctgaaaggactcgacgatctcatgagcggactgggcgcagccgggaaggctgttggcgtcgccatcggagcggtcggaggagcagtggctagcgtggtggagggcgtggccaccttcctgaagaaccctttcggcgcctttaccatcatcctggtggccatcgccgtggtcatcattacatatctgatttatacaagacagagaaggctctgcacccagcccttgcagaacctgttcccctacctggtcagtgccgacggtacaaccgtgaccagcggtagcaccaaggacacctccctgcaggcaccgccgagctacgaggagtccgtgtataacagtggaagaaagggccccggaccgcccagcagcgacgcatccaccgccgctcctccctacacaaatgagcaggcctatcagatgttgctggctctggcacgcctggacgccgagcagcgagctcagcagaacggcaccgattccctggatggacgcacaggcacacaggacaaggggcagaagcccaacctcctcgacagactgagacaccggaagaacggatacaggcatctgaaggactccgatgaggaggagaacgtt

SEQ ID NO: 95 - DNA, 2721 bp, synthetic construct
  misc_feature 1..2721: note = Synthetic polynucleotide
  source 1..2721: mol_type = other DNA
atggaatccaggatctggtgcctcgtggtctgtgtgaacttgtgcatcgtgtgcttgggtgccgccgtgagcagtagcagcaccagaggcaccagcgcaacacactcacaccacagctcccataccacttccgccgcccactccagatcgggctccgtgagccagagggtcaccagcagccagacggtgtcccacggagtgaatgaaaccatctacaacactactctgaagtacggagacgtcgtcggcgtgaataccactaagtacccctacagggtctgctctatggcccaaggcacagacctgatcagatttgaaagaaatatcgtctgtacctccatgaagcccatcaatgaggacttagacgagggcattatggtggtgtataaacgcaacattgtggcccacactttcaaggtcagagtgtatcagaaagtgctcaccttcaggcgtagctatgcctatatccacaccacttatctcctcggcagcaacaccgagtatgttgccccgcctatgtgggagattcaccatataaatagccatagccagtgctacagctcctattcgagagtaatcgccggaaccgttttcgtcgcctaccacagagactcgtacgagaacaagacaatgcagctgatgccagatgactattcgaacacccacagcacgagatatgtcaccgtgaaagatcagtggcacagcaggggtagtacatggttgtatagggaaacctgcaatctcaattgcatggtgaccatcaccaccgccagaagcaaatacccctatcatttcttcgctacctcgacaggagacgtggtggacatatctcccttttataatggcacaaatagaaatgctagctactttggagagaacgccgacaaattcttcatcttccctaactataccatcgtgagcgactttgggcgacctaacagcgccctcgagactcacaggctggtggctttcttagagagggctgatagtgttatctcttgggacattcaggatgagaagaacgtgacatgccagctgacattttgggaggctagcgagcgaaccatcaggtccgaggccgaggacagctaccatttctctagtgccaagatgaccgccaccttcttgtcaaagaagcaagaggtgaacatgtccgactctgcgctggactgtgtccgcgacgaggcaattaataaactgcagcagatctttaataccagctacaaccagacatacgagaagtatggcaacgtgagcgtcttcgaaaccacaggcggccttgtcgtcttttggcaaggcatcaagcagaagagtctggtggagctggaaagactcgccaaccggtcatccctgaatctgacccacaataggacaaagcgcagcaccgatgggaacaacgccacccacctgtcgaacatggagtcagtgcacaacctggtgtacgcccagctgcagttcacttatgataccctcagaggctacattaaccgcgcactggctcagatcgccgaagcatggtgcgtggaccagcggcgaaccctggaagtgtttaaagagctctccaagattaatcctagcgccatcctgagtgctatctacaataagcctatcgccgcaagatttatgggcgacgtgctgggactggcttcctgcgtgacaattaaccagacctccgtcaaggtgctgagggacatgaacgtgaaggagagccccggcagatgctatagccggccagtggtgatcttcaatttcgccaacagctcatacgtgcagtacggccagctcggggaggataatgaaatcctgctgggaaatcacagaaccgaggagtgtcagctgcccagtctgaagattttcatcgcaggcaacagtgcctatgaatacgtggactatctgttcaaacgcatgatcgatctgagctctatctccaccgtggactccatgattgccttggatatcgacccactggagaacaccgatttcagagtgctggagctgtacagccagaaggagctcaggtccagcaatgtgttcgacctggaggaaatcatgagagagttcaactcctacaaacagagagtcaagtacgtggaggacaaggtggtggatcccctgcctccctacctgaaggggctggacgacctgatgagtggcctgggagccgccggcaaagctgtgggagtggccatcggtgccgtcggaggggctgtggccagcgtcgtcgagggagttgccacattcctgaagaaccccttcggggccttcaccattatcctagtcgccattgccgtggtcatcattacctatctgatctacacgcggcagagacggctgtgcacccagcctttgcagaacctgttcccctatttagtgtccgctgacgggaccactgtgacaagcggaagcaccaaggacacatccctgcaggccccacccagctacgaggagtctgtttacaattctggccggaagggccccggccctccctcttctgacgcctccaccgcagcccctccttacacaaacgagcaggcttaccagatgctgttggctttggcccgtctggacgccgaacagagggcccagcagaatggcaccgactccttggacggccggacagggacccaggataagggtcagaagcctaacctactggatcggctccgccatcgcaagaatggctacagacatctcaaggacagcgacgaagaagagaatgtg

SEQ ID NO: 96 - DNA, 2721 bp, synthetic construct
  misc_feature 1..2721: note = Synthetic polynucleotide
  source 1..2721: mol_type = other DNA
atggaatcaagaatctggtgtctcgtggtgtgcgtgaacctgtgtatcgtctgtcttggcgccgccgtctcttcctcaagcacccggggtaccagtgccacccactcacatcactcctcccacactaccagcgccgcccacagcagatccggctccgtgtcccagcgggtgaccagcagccagaccgtgtcacacggcgttaatgaaaccatttacaacaccacactgaagtacggggacgtggtgggcgtgaacaccaccaagtatccctacagggtgtgcagcatggcccagggcaccgacctgattcggttcgagagaaacatcgtgtgcacatccatgaagcctatcaatgaggacctcgacgagggcatcatggtggtttacaagaggaacattgtcgcacacacatttaaggtgcgagtgtaccagaaggtgttaaccttcagaaggtcctacgcatacatccacaccacctacctcctgggctctaacacagaatacgtcgcccctcccatgtgggagattcaccacatcaacagtcacagccagtgctacagctcgtatagcagagttatcgctggcaccgtgttcgtggcttatcaccgcgacagctacgagaacaagacgatgcaacttatgcccgacgattactcaaacacgcactccactagatacgtgactgtgaaggaccagtggcacagtagaggcagcacctggctgtaccgggaaacatgcaatctcaattgtatggtcaccattaccaccgccaggtccaagtacccttaccacttcttcgccacctccactggcgacgtggtcgacatcagccccttctacaatggcaccaacaggaacgcctcttactttggggagaacgccgataaattctttattttccccaactacactattgtctccgactttggcagacccaactcagcattggaaacccacaggctcgtggccttcctggagcgggccgatagtgtgatcagctgggacatccaggatgagaagaacgtgacatgccagctgaccttctgggaggccagcgaacgaaccatccggtccgaggccgaggactcttatcacttctctagcgcaaagatgaccgccaccttcctgtctaagaaacaggaggtgaacatgagcgacagcgccctggactgcgtcagagacgaggcaatcaacaagctgcagcaaatcttcaacaccagctacaaccaaacctacgagaaatacggcaacgtcagcgtcttcgagactaccggagggctcgttgttttctggcagggcattaagcagaagtctctggtcgagctggaaaggctggccaatagaagctccctaaacctcactcacaacagaactaagagaagcaccgatggcaataacgccactcatctgagtaacatggagtctgttcacaacctggtgtatgcccagctgcagtttacttatgacacactgaggggctacatcaatcgagccctggcccagatcgccgaggcttggtgcgtcgaccagagaagaacactggaagtgttcaaggagctgagtaagattaatcccagcgccattctgtccgccatctacaataagccaatcgccgcaagattcatgggtgacgtgctgggcctggcctcctgcgtgacaatcaaccagacaagcgtgaaagtcctcagagacatgaacgtcaaggagtctcctggcaggtgttactcccggcccgtggtgatatttaatttcgccaacagcagttacgtgcagtacggacagctgggcgaggataacgagatactgctcggaaaccatagaacagaggagtgccaactgccctccctgaagattttcatcgccgggaacagcgcctatgagtatgttgactatctgttcaagcggatgatcgacctgagttctatcagcaccgttgactccatgattgctctcgatatcgatcctctggagaacaccgatttcagagtgctggaactctactctcagaaagagctaagaagctcgaacgtgttcgacctggaagaaatcatgagagagttcaactcctacaaacagagggttaagtacgtagaggataaggtcgtggaccctctgcctccataccttaagggattagatgatctgatgagcggcctgggcgctgccggaaaggccgtgggagtggccatcggcgcagtgggtggtgccgtggctagcgtcgtggaaggcgttgccacattcttgaagaacccattcggggccttcacaatcatcctggtggctatcgccgttgtgattatcacatatctgatctacactcgccagcggaggctctgcacccagcctctgcagaaccttttcccctacctagtgtccgctgatgggactacagtcactagcggcagcactaaggacacatccctgcaggctcctccatcttacgaggagagcgtgtataactccgggcgcaagggacctggccctcccagcagcgacgccagcacggcggctcctccctacaccaacgagcaggcataccagatgttgcttgcactggcccgtctggacgctgagcagagggcccagcagaatgggactgattccctggacggcagaaccggcacacaggataaaggacagaaaccgaatctgctcgacaggctgaggcaccggaagaatggatacaggcatctgaaggacagtgacgaggaggagaacgtg

SEQ ID NO: 97 - DNA, 2721 bp, synthetic construct
  misc_feature 1..2721: note = Synthetic polynucleotide
  source 1..2721: mol_type = other DNA
atggagtcaagaatctggtgcttggtggtgtgtgtgaacttgtgtatcgtgtgccttggagccgccgtgagcagcagctccaccagaggcaccagcgccacccacagccatcactcttcccacaccacaagcgccgcccactcgcggagcgggagtgtttcccaacgggtgacaagcagccagactgtgagccacggcgttaacgagacaatctacaacacaacactgaagtacggcgacgtggtgggtgtaaatactaccaagtatccttacagggtgtgctctatggcccagggtaccgacctgatcaggtttgagagaaacattgtttgcacaagcatgaagcccatcaatgaggacttggatgagggcatcatggtggtttacaagagaaatatcgtggcccacaccttcaaagtgagggtgtatcagaaggtgctgacctttagaaggagctacgcttatatccacacaacctaccttctgggcagcaacaccgagtacgtcgcaccacccatgtgggaaattcaccacatcaactctcactcccagtgctattccagctacagcagagtgatagccggcacagtcttcgtggcctaccacagggatagttacgagaataagacgatgcaactgatgcctgacgattactccaacacacacagcacccggtacgtcaccgtgaaggaccagtggcactccagaggtagtacttggctgtaccgggagacttgtaacctgaactgcatggtgacaattaccactgctcgaagcaagtacccttaccacttctttgccacctctaccggcgatgtcgtagacatatctcctttctataacgggaccaacagaaacgcttcgtacttcggcgagaacgctgacaagttcttcatcttcccgaactacactatagttagcgactttggtaggccgaacagcgccctggagacacaccgacttgtggccttcctcgagagagctgacagcgtgatctcctgggacatccaggacgagaagaacgtcacctgccagctgacattctgggaggcctctgagaggaccatcagatccgaggccgaggattcataccactttagcagcgctaagatgaccgctaccttcctgagtaagaagcaggaagtgaacatgtccgactcagccctcgactgcgtgagggacgaggccatcaacaagctgcagcagatcttcaacacctcctacaaccagacatatgagaagtatggtaacgtgagcgtgttcgagacaaccggcggactggtcgtgttttggcagggcataaagcagaagtctctggtcgagctggagaggctggcgaacaggagcagcctcaacctgacccataacagaaccaaacgcagcaccgacggcaacaatgctacccacctgtcaaacatggagagcgtccacaacctggtgtatgcccagctgcaatttacatacgacacgctgcgcggctacatcaatagagccctggcccagatcgccgaggcttggtgcgttgaccagcggcgtactctggaagtcttcaaggagctgagcaagatcaatcccagcgctatcctgagcgcgatctacaataaacctattgctgccagattcatgggagacgtgttggggctggccagctgcgtgacaatcaatcagaccagcgtgaaagtgctgagagacatgaatgtgaaggagtctcctggtaggtgctactcaaggcccgtcgtaattttcaatttcgccaacagttcctacgtgcagtacggacagctgggcgaagacaatgagatcctcctgggcaaccatcggacggaggagtgtcaactcccatcactgaagatctttatcgcaggcaattccgcctatgagtatgtggactatctgttcaagaggatgatcgacctgtccagcatcagcacagtggattcaatgattgcccttgacatcgaccctcttgagaataccgactttagagtgctggagctttatagccagaaagagctcaggagctccaatgtgttcgacctggaagagatcatgcgggagtttaacagctacaagcagagggttaaatatgtggaggacaaggttgtggatccactgccgccctacctgaaagggctggacgacctcatgtccggcctaggagccgcagggaaagccgtgggcgtggccatcggcgcagttggaggcgccgtcgcctctgtggttgaaggcgttgcgacctttctgaagaacccattcggcgccttcaccattatcctggtggccattgccgtggtcatcatcacctatctgatctacaccaggcaacgacgcctgtgcacccagcccctgcagaacctgttcccttacctggtcagcgccgatgggaccacagtgacctctggttctactaaagacaccagccttcaggcccctccatcctacgaggagtctgtgtacaatagcggcagaaagggccccggcccgcccagcagcgatgccagcaccgccgctcctccatacacgaacgagcaggcctatcagatgctgctggcccttgcccgcctggacgccgagcagcgtgctcagcagaatggcaccgattctctggacggccgaactggaacgcaagacaagggacagaagccaaacctgctggacagactgagacacaggaagaatggctacaggcatctgaaggattcagacgaggaggagaacgtg

SEQ ID NO: 98 - DNA, 2721 bp, synthetic construct
  misc_feature 1..2721: note = Synthetic polynucleotide
  source 1..2721: mol_type = other DNA
atggagagccggatctggtgccttgtggtgtgcgtgaacctttgcatcgtgtgcctcggcgccgccgtgagctcatcgagcacccggggcaccagcgccacccacagccaccacagcagccacaccaccagcgcggcccacagtcggagcggcagcgtgagccagcgggtgacctcctcccagaccgtctcccacggcgtgaacgaaaccatctacaacaccaccctgaagtacggcgacgtggttggggtaaataccactaagtacccctaccgggtgtgcagcatggcccagggcaccgacctgatccggttcgagcggaacatcgtctgtaccagcatgaagcccatcaacgaggacctggacgagggcatcatggttgtctacaagcggaatatcgtagcccacaccttcaaggtgcgggtgtaccagaaggtgctgaccttccggcggagctacgcctacattcacacgacttacctgctgggcagcaacaccgagtacgtggcgccgcccatgtgggagatccaccacatcaactctcactctcagtgctacagcagctacagccgggtgatcgccggcaccgtgttcgtggcctaccaccgggacagctacgagaacaagaccatgcagctgatgcccgacgactattctaacacacactccactaggtacgtgaccgtgaaggaccagtggcactccagaggcagcacctggctgtaccgggagacgtgcaacctgaactgcatggtgaccatcaccaccgcccggtcaaagtacccttaccacttcttcgccaccagcactggggatgtggttgacatcagccccttctacaacggcaccaaccggaacgccagctacttcggcgagaacgccgacaagttcttcatcttccccaactacaccatcgtgagcgacttcggccggcccaacagcgccctggaaacacaccggctggtggccttcctggagcgggccgacagcgtgatcagctgggacatccaggacgagaagaacgtgacctgccaactcacattttgggaggccagcgagcggaccatccggagcgaggccgaggactcctatcacttcagcagcgccaagatgaccgccaccttcctgagcaagaagcaggaggtgaacatgagcgattcggcattggactgcgtgcgggacgaggccatcaacaagctgcagcagatcttcaacaccagctacaaccagacctatgagaaatacggcaacgtgagcgtgttcgaaaccaccggcggactggttgttttctggcagggtatcaagcagaagagtttggtggagctcgagcgcctggcaaacaggagcagcctgaacctgacccacaaccggaccaagcggagcaccgacggcaacaatgcaacgcacctatccaacatggagtccgtgcacaacctggtgtacgcccagctgcagttcacctacgacaccctgcggggctacatcaaccgggccctggcccagatcgccgaggcatggtgcgtggaccagcggcggaccctggaggtgttcaaggagctttccaagatcaacccctctgccatcctgtctgctatctacaataagccaatcgcggcacgcttcatgggagacgtactgggcctggccagctgcgtgactattaatcagactagcgtcaaagtgctacgggacatgaacgtaaaggagagccccggccggtgctattctcggcccgtggtcattttcaacttcgccaacagttcctacgtgcagtacggacagttaggcgaggacaacgagattctgctgggtaaccaccggaccgaggagtgccaacttcccagtctaaagatatttatcgccgggaattccgcttatgagtatgtcgactacctgttcaagcggatgatcgacctgtccagtatcagcaccgtggacagcatgattgcactggatatcgaccctctcgagaacaccgacttccgggtgctggagctgtacagccagaaagagctgagatcaagtaatgtctttgacctggaggagatcatgcgggagttcaatagctacaagcagagggtgaaatatgtcgaagacaaggtagtagacccgctgcctccctacctgaaggggcttgacgacctcatgtcagggttaggggcagctggcaaggccgttggcgtcgccatcggcgcggtgggcggtgccgttgcctccgtggtcgaaggcgtcgctaccttcctcaagaaccccttcggcgccttcaccatcatcctggtggctattgcagttgtcatcattacctacctcatctacacccggcagcggaggctgtgcacccagcccctgcagaacctgtttccatacctggtgagcgcagacggaactaccgtgacgagcggatccactaaggacaccagcctgcaggcgcctccttcatacgaagagagtgtgtacaacagcggccggaagggccccggacctccgagtagcgacgcaagtaccgccgccccaccctataccaacgagcaagcttaccagatgctgctggcacttgctcggctggacgccgaacaacgcgcccagcagaacggaactgattctctggacggccggaccggcacccaggacaagggccagaagcccaacctgttggaccggctgcggcaccggaagaacggctatcgtcacctgaaagacagcgacgaggaggagaacgtg

SEQ ID NO: 99 - DNA, 2721 bp, synthetic construct
  misc_feature 1..2721: note = Synthetic polynucleotide
  source 1..2721: mol_type = other DNA
atggagagccggatctggtgcctagtggtgtgcgtgaacctctgcatcgtgtgcctaggcgccgccgtgagcagttctagtacccggggcaccagcgccacccacagccaccacagcagccacactacgtcagcagcgcatagtcggagcggcagcgtgagccagcgggtgacgtcttcccagacagtgtcccacggcgtgaacgaaaccatctacaacaccaccctgaagtacggcgacgtggtgggtgtcaatacaactaagtacccctaccgggtgtgcagcatggcccagggcaccgacctgatccggttcgagcggaatattgtgtgtaccagcatgaagcccatcaacgaggacctggacgagggcatcatggtggtatacaagagaaacattgtcgcccacaccttcaaggtgcgggtgtatcagaaggtgctgaccttccggcggagctacgcctacattcatacgacttacctgctgggcagcaacaccgagtacgtggccccgcccatgtgggagatccaccacatcaacagccactcccagtgctacagcagctacagccgggtgatcgccggcaccgtgttcgtggcctaccaccgggacagctacgagaacaagaccatgcagctgatgcccgacgactatagcaatactcacagcacacggtacgtgaccgtgaaggaccagtggcacagccgcggcagcacctggctgtaccgggaaacgtgcaacctgaactgcatggtgaccatcaccaccgcccggtcgaagtatccctatcacttcttcgccaccagcacgggcgatgtggttgacatcagccccttctacaacggcaccaaccggaacgccagctacttcggcgagaacgccgacaagttcttcatcttccccaactacaccatcgtgagcgacttcggccggcccaacagcgccctggaaacccaccggctggtggccttcctggagcgggccgacagcgtgatcagctgggacatccaggacgagaagaacgtgacctgccagcttacattctgggaggccagcgagcggaccatccggagcgaggccgaggacagttaccacttctcgagcgccaagatgaccgccaccttcctgagcaagaagcaggaggtgaacatgagcgacagtgctctggactgcgtgcgggacgaggccatcaacaagctgcagcagatcttcaacaccagctacaaccagacctatgagaaatacgggaacgtgagcgtgttcgagacaaccggcggcttagtagtgttctggcaggggatcaagcagaagagtttggtggagctcgagcggctggcgaacagaagcagcctgaacctgacccacaaccggaccaagcggagcaccgacggcaacaacgcaacgcacttatcaaacatggaaagtgtgcacaacctggtgtacgcccagctgcagttcacctacgacaccctgcggggctacatcaaccgggccctggcccagatcgccgaggcgtggtgcgtggaccagcggcggaccctggaggtgttcaaggagttgtcgaagatcaacccttctgccatcctgtcagcaatttacaataaacctattgccgcaaggttcatgggagatgtcctgggcctggccagctgcgtgaccataaaccagacaagcgtcaaagtcctccgggacatgaatgtgaaagagagccccggccggtgttacagtcgacccgtggtgatctttaacttcgccaattcttcttatgtgcagtacggacagctcggcgaggacaacgagatcctgctcggtaaccaccggaccgaggagtgtcagcttccctcactgaagattttcattgcggggaacagtgcatacgagtatgttgactacctgttcaagcggatgatcgatctgtctagtatcagcaccgtggacagcatgatcgctctggatatcgacccattggagaacaccgacttccgggtgctggagctgtacagccagaaggagcttcgcagcagtaatgtgtttgacctggaggagatcatgcgggagttcaattcttacaagcagcgcgtgaaatacgttgaggacaaggtggtcgatccgctgcctccctacctgaagggcctggatgatctcatgagcgggttaggggctgccggcaaggccgtcggcgttgccatcggcgcagtgggcggagccgtcgccagcgtggtggagggtgttgcaacgttcctgaagaaccccttcggcgccttcaccatcatcttggttgcaatcgcggttgttatcattacctaccttatctacacccggcaacggcggctgtgcacccagcccctgcagaacctgtttccatacttggtgagcgcggatgggaccaccgtgacttcaggttccaccaaggacaccagcctgcaggcgcctccctcatacgaggagtccgtatacaacagcggccggaaggggccaggtcctcctagctcggacgcaagtactgccgcaccgccttataccaacgagcaggcatatcagatgctgcttgccctggctcggctggacgccgaacagcgcgcccagcagaacggaacagattccctggacggccggaccggcacccaggataagggccagaagcccaacttgctggaccggctgcggcaccggaagaacggctataggcatctgaaggacagcgacgaggaggagaacgtg

SEQ ID NO: 100 - DNA, 2721 bp, synthetic construct
  misc_feature 1..2721: note = Synthetic polynucleotide
  source 1..2721: mol_type = other DNA
atggagagccggatctggtgcctagtggtgtgcgtgaacctatgcatcgtgtgcttaggcgccgccgtgagctcatcgtccacccggggcaccagcgccacccacagccaccacagcagccacacgacaagcgccgcccactcccggagcggcagcgtgagccagcgggtgacttcttcccagacagtgagccacggcgtgaacgagactatctacaacaccaccctgaagtacggcgacgtggtgggcgtcaacactaccaagtacccctaccgggtgtgcagcatggcccagggcaccgacctgatccggttcgagcggaacattgtgtgcaccagcatgaagcccatcaacgaggacctggacgagggcatcatggttgtgtacaagcgtaatatcgtcgcccacaccttcaaggtgcgggtgtaccagaaggtgctgaccttccggcggagctacgcctacatccatactacgtacctgctgggcagcaacaccgagtacgtggctcctcccatgtgggagatccaccacatcaactcccatagccagtgctacagcagctacagccgggtgatcgccggcaccgtgttcgtggcctaccaccgggacagctacgagaacaagaccatgcagctgatgcccgacgactattcgaacacccactcaaccagatacgtgaccgtgaaggaccagtggcattcacggggcagcacctggctgtaccgggaaacatgcaacctgaactgcatggtgaccatcaccaccgcccggagtaaatacccttatcacttcttcgccaccagcacgggcgacgtcgtagacatcagccccttctacaacggcaccaaccggaacgccagctacttcggcgagaacgccgacaagttcttcatcttccccaactacaccatcgtgagcgacttcggccggcccaacagcgccctggagacacaccggctggtggccttcctggagcgggccgacagcgtgatcagctgggacatccaggacgagaagaacgtgacctgccagctgacgttttgggaggccagcgagcggaccatccggagcgaggccgaagattcctatcactttagcagcgccaagatgaccgccaccttcctgagcaagaagcaggaggtgaacatgtctgattccgcgctggactgcgtgcgggacgaggccatcaacaagctgcagcagatcttcaacaccagctacaaccagacctatgagaagtatgggaacgtgagcgtgttcgagacaaccggcgggctggtcgtcttctggcaaggcattaagcagaagtccctcgtggagctggaacggctggccaaccgtagcagcctgaacctgacccacaaccggaccaagcggagcaccgacggcaacaatgctactcatctatcaaacatggaaagcgtgcacaacctggtgtacgcccagctgcagttcacctacgacaccctgcggggctacatcaaccgggccctggcccagatcgccgaggcctggtgcgtggaccagcggcggaccctggaggtgttcaaggagctaagtaagatcaacccctccgcaatcctgagcgccatctataacaagcctatcgccgcccggttcatgggcgatgtgctgggcctggccagctgcgtcaccatcaatcaaactagcgtgaaggtcctacgggacatgaacgtgaaagagagccccggccggtgctactcccggcccgtggtcatcttcaatttcgccaactcttcctatgtgcagtacgggcagctgggcgaggacaacgagattctgctgggtaaccaccggaccgaggagtgccagcttccctccctcaagattttcatagcaggcaattctgcctatgaatacgttgactacctgttcaagcggatgatcgatctctctagtatcagcaccgtggacagcatgattgcgttggacatcgacccgttagagaacaccgacttccgggtgctggagctgtacagccagaaagaactgcgttcaagcaacgttttcgacctggaggagatcatgcgggagttcaactcttacaagcagcgggtcaagtacgtcgaggataaggtcgtggacccgctgccgccctacctgaagggactggacgatctgatgtccggattgggagctgcaggaaaggccgtgggagtagccatcggcgctgttggaggggcagtggccagcgtggtcgaaggcgtcgcgacgttcctgaagaaccccttcggcgccttcacaataatcttggttgccattgctgtcgtcattattacatatcttatctacacccggcagagaagactgtgcacccagcccctgcagaacctgttcccttatttggtgagcgccgacgggacaaccgtcacctccggctcaacgaaggacaccagcctgcaggctccgccttcatatgaagagtcagtatataacagcggccggaaggggccaggtcctccatctagcgacgcatcaactgccgcacctccgtacaccaacgagcaggcataccagatgctgttggccctcgcacggctggacgccgagcaacgcgcccagcagaacgggacggactctttggatggccggaccggcacccaagacaagggccagaagcccaatttgctggaccggctgcggcaccggaagaacggctatagacatctgaaggacagcgacgaggaggagaacgtg

SEQ ID NO: 101 - DNA, 2721 bp, synthetic construct
  misc_feature 1..2721: note = Synthetic polynucleotide
  source 1..2721: mol_type = other DNA
atggagagccggatctggtgcctagtggtgtgcgtgaacctatgcatcgtgtgccttggcgccgccgtgagctcgtccagtacccggggcacctccgccacccactcccaccactcctcccacactacaagcgccgcccactcgcgctccggctccgtctcccagcgcgtcaccagttcccagaccgtgagtcacggcgtcaacgaaaccatctacaacaccaccctcaagtacggcgacgtcgtgggcgtgaatacaaccaagtacccctaccgcgtctgctccatggcccagggcaccgacctcatccgcttcgagcgcaacatcgtctgcacctccatgaagcccatcaacgaggacctcgacgagggcatcatggtcgtgtataagcgaaatattgtggcccacaccttcaaggtccgcgtctaccagaaggtcctcaccttccgccgctcctacgcctacattcacacaacctacctcctcggctccaacaccgagtacgtcgcccctcccatgtgggagatccaccacatcaacagtcacagccagtgctactcctcctactcccgcgtcatcgccggcaccgtcttcgtcgcctaccaccgcgactcctacgagaacaagaccatgcagctcatgcccgacgactatagcaatacacatagtacccgctacgtcaccgtcaaggaccagtggcacagcaggggctccacctggctctaccgcgagacttgcaacctcaactgcatggtcaccatcaccaccgcccgctcaaagtacccgtatcacttcttcgccacctccacgggagacgtggtggacatctcccctttctacaacggcaccaaccgcaacgctagctatttcggcgagaacgccgacaagttcttcatcttccccaactacaccatcgtctccgacttcggccgccccaactccgccctcgaaacccacaggcttgtggccttcctcgagcgcgccgactccgtcatctcctgggacatccaggacgagaagaacgtcacctgccagctcacattctgggaggcctccgagcgcaccatccgctccgaggccgaggattcgtaccactttagctcagcaaagatgaccgccaccttcctctccaagaagcaggaggtcaacatgagcgactctgctttggactgcgtccgcgacgaggccatcaacaagctccagcagatcttcaacacctcctacaaccagacttatgagaagtatggcaacgtctcggtgttcgagactacgggcggtctggtggtcttctggcaggggattaagcagaagtccctcgtcgagttggagagactcgccaaccgctcctccctcaacctcacccacaaccgcaccaagcgctccaccgacggcaacaacgccacgcacctctcaaacatggagtccgtccacaacctcgtctacgcccagctccagttcacctacgacaccctccgcggctacatcaaccgcgccctcgcccagatcgccgaggcctggtgcgtcgaccagcgccgcaccctcgaggtcttcaaggagctcagtaagatcaacccaagtgcgatcctgtcggccatttacaataaaccgattgcagcccgcttcatgggtgacgtactcggcctcgcctcctgcgtgacgattaatcagaccagcgtcaaggtgcttcgcgacatgaatgtgaaggagagcccaggccgctgttacagtcggcccgtcgtcattttcaatttcgccaatagcagctatgtccagtacggccagctcggcgaggacaacgagatactccttggcaaccaccgcaccgaggagtgccagctgccgtctctgaagatattcatagccggcaacagcgcttatgaatacgtggactacctcttcaagcgcatgatcgacctctcctccatctccaccgtcgactccatgatcgcacttgatatcgacccactggagaacaccgacttccgcgttctggaactctactcccagaaggagctacggtcctccaatgttttcgacctcgaggagatcatgcgcgagttcaattcatacaagcaacgggtgaagtatgtggaggacaaggtcgtcgatcctctgcctccctacctcaagggtcttgatgatctcatgtccggcctcggcgctgccgggaaggcagtgggagtcgccatcggcgccgttggaggggccgtcgcctctgtggtggagggcgtggctaccttcctgaagaaccccttcggcgccttcaccattattctggtggccatcgcagtggttatcatcacgtaccttatctacacccggcagagaaggctctgcacccagcccctccagaacctctttccttatctcgtcagcgcagacggtacaacagttactagtggaagtaccaaggacacctccctccaggccccgccaagctacgaggaaagtgtttacaactccggccgcaagggccccggcccaccttcttccgacgcttccaccgctgctccaccatacaccaacgagcaggcctaccagatgctcctggcactggctcggctggatgccgagcagagggcccagcagaacggtaccgattccctcgacggccgcaccggcacccaggataagggccagaagcccaatttactagacagactgcgccaccggaagaacggctaccgccatctgaaggactccgacgaggaggagaacgtc

SEQ ID NO: 102 - DNA, 2721 bp, synthetic construct
  misc_feature 1..2721: note = Synthetic polynucleotide
  source 1..2721: mol_type = other DNA
atggagagccggatctggtgcctagtggtgtgcgtgaacctctgcatcgtgtgcttgggcgccgccgtgagtagttccagtacccggggcacctccgccacccactcccaccactcctcccacactacgagtgccgcccactcacgctccggctccgtctcccagcgcgtcactagttctcagacagtgtctcacggcgtcaacgagacaatctacaacaccaccctcaagtacggcgacgtcgtgggtgtgaatactactaagtacccctaccgcgtctgctccatggcccagggcaccgacctcatccgcttcgagcgcaacatcgtctgcacctccatgaagcccatcaacgaggacctcgacgagggcatcatggtcgtctacaagagaaatatagtggcccacaccttcaaggtccgcgtctaccagaaggtcctcaccttccgccgctcctacgcctacattcacactacctacctcctcggctccaacaccgagtacgtcgccccacccatgtgggagatccaccacatcaactctcacagtcagtgctactcctcctactcccgcgtcatcgccggcaccgtcttcgtcgcctaccaccgcgactcctacgagaacaagaccatgcagctcatgcccgacgactactcaaacactcacagcacccgctacgtcaccgtcaaggaccagtggcacagccgcggctccacctggctctaccgcgagacttgcaacctcaactgcatggtcaccatcaccaccgcccgctcgaagtatccgtaccacttcttcgccacctccacgggcgatgtggtcgacatcagtccattctacaacggcaccaaccgcaacgcttcatacttcggcgagaacgccgacaagttcttcatcttccccaactacaccatcgtctccgacttcggccgccccaactccgccctcgagacacaccgcctggtcgccttcctcgagcgcgccgactccgtcatctcctgggacatccaggacgagaagaacgtcacctgccagttgaccttctgggaggcctccgagcgcaccatccgctccgaggccgaggacagttaccatttcagtagtgccaagatgaccgccaccttcctctccaagaagcaggaggtcaacatgtcggactctgctcttgactgcgtccgcgacgaggccatcaacaagctccagcagatcttcaacacctcctacaaccagacttatgagaagtatggtaacgtctctgtgtttgagactacaggcggtcttgtcgtcttctggcagggtatcaagcagaagtccctcgtcgagctggaacgcctcgccaaccgctcctccctcaacctcacccacaaccgcaccaagcgctccaccgacggcaacaacgcaacacatcttagcaacatggagtccgtccacaacctcgtctacgcccagctccagttcacctacgacaccctccgcggctacatcaaccgcgccctcgcccagatcgccgaggcctggtgcgtcgaccagcgccgcaccctcgaggtcttcaaggagctgagtaagatcaacccaagtgcaatcctaagcgctatttacaacaaacctatcgcagccaggttcatgggagacgtcctcggcctcgcctcctgcgtcaccattaatcagacgtctgttaaggttctccgcgacatgaacgtgaaagagtctccgggccgctgctacagcaggcccgtcgtcatcttcaatttcgccaattcttcatatgtccagtacggccagctcggcgaggacaacgagattctcttagggaaccaccgcaccgaggagtgtcagctacccagcctgaagatctttattgccggcaatagcgcttatgagtatgttgactacctcttcaagcgcatgatcgacctctcctccatctccaccgtcgactccatgatcgctctggatatcgaccctctggagaacaccgacttccgcgtgcttgagctctactcccagaaagagcttaggtcaagcaacgttttcgacctcgaggagatcatgcgcgagttcaactcatataagcaacgcgttaaatatgtagaggataaggtggttgatccacttcctccctacctcaagggtctggatgacctcatgtccggcctcggggcagcaggcaaggccgtcggcgttgccatcggcgccgtgggaggtgctgtggccagtgttgtcgagggcgtagccaccttcttaaagaaccccttcggcgcctttacaataatcctggtggccatcgctgtggttatcattacctatcttatctacaccaggcagcggaggctctgcacccagcccctccagaacctcttcccttacctcgtgagcgcggacgggacgaccgtcacatctggcagtacaaaggacacctccctccaggccccgcctagttatgaagagagcgtttacaactccggccgcaagggccccggtcctccctcctccgacgccagcaccgcagcgcctccatacaccaacgagcaggcctaccagatgctcttggccctggcccgactggatgccgagcagcgcgcccagcagaacggaaccgactccctcgacggccgcaccggcacccaggacaaaggccagaagcccaatctgctcgaccgcctgcgacaccgcaagaacggctatcggcaccttaaagactccgacgaggaggagaacgtc

SEQ ID NO: 103 - DNA, 2226 bp, synthetic construct
  misc_feature 1..2226: note = Synthetic polynucleotide
  source 1..2226: mol_type = other DNA
atgcggccaggacttcctagctacctaatcatccttgccgtgtgtcttttctcacacctcctctctagtaggtacggcgcagaggccgtgtccgagccactcgacaaggccttccaccttttgttaaacacctacggtcggccaatcagattcctccgcgagaacaccacccagtgcacgtacaattcgtctctccgcaacagcacagtcgtgagggagaacgctatatcattcaacttcttccagtcttacaaccagtattacgtgttccacatgccaagatgcctgttcgctggcccactggccgagcagttccttaaccaggtggatctgacagagactctggagagataccaacagaggctgaacacctacgcactggtgagcaaggatctggcctcctacaggagcttcagccaacagctgaaggcccaggacagtctgggcgagcaaccaaccaccgtacctccacctattgacttatcaatacctcacgtgtggatgcctcctcagaccactcctcacggctggaccgaatcccacaccacctccggcctgcacaggcctcatttcaaccagacctgtattctcttcgacggacacgacctgcttttcagcaccgtcacgccatgcttgcaccagggcttctacctgatcgacgagctcaggtatgtgaagatcactctgaccgaggacttcttcgtggttactgtgagcatcgatgacgataccccaatgttactgatcttcggccacctgcctagggtgctgttcaaggcaccataccagagagacaatttcatcctgagacaaaccgagaagcacgagctgctggtgctggtcaagaaggaccagctgaataggcactcatacctgaaggacccagatttcctggacgctgcccttgatttcaattacctggacctgtccgccctgctgagaaacagcttccacagatacgccgtggatgtgctgaagtcaggcagatgtcagatgttggaccgccgaaccgttgagatggccttcgcctacgcgctggccctgttcgccgccgctcggcaggaggaagctggcgcacaggtgagcgtgccgagggctctggaccgacaggctgctctgctacagattcaggaattcatgatcacctgcctgtcacagactccgcctcggactaccctgctgctgtatcctacagcagtggacctggcaaagagagctctttggacccctaaccagatcaccgatatcaccagcctcgtccggctggtctacattctgtctaagcagaatcagcagcacctgatccctcagtgggctctcagacagatcgccgatttcgccctgaagctgcacaagacccacctggcttctttcctgagcgctttcgccagacaggaactgtacctgatgggatcgcttgtgcacagcatgctggtgcacacaactgagaggagagagattttcattgtggagactggcctgtgcagcctggccgagctgtcccatttcacccagctcctcgctcatccgcaccacgagtacctctccgacctctataccccttgctcctcttccggccggcgcgatcacagcctggaaagactcactagactgttcccagacgctaccgtgccggctactgtcccggcagcactgagcatcctgagcactatgcagccttctacgctggaaaccttcccggacctgttctgcctgccactcggagaaagcttctctgccctgacggtgagtgagcacgtgtcgtacatcgtgacaaaccagtacctgatcaagggtatcagctacccagtgtccacaactgtggtgggccagagcctgatcatcacccagaccgatagccaaacaaagtgcgaactgacaagaaacatgcataccactcattccatcactgtggccttaaacatctccctggagaactgcgccttctgtcagtccgccctgctggagtacgatgatacccagggcgttattaatatcatgtacatgcatgatagcgacgatgtgcttttcgccctggacccatacaacgaggtggtggtgtccagccctagaacccactacctcatgctgctgaagaacggcacagtgctggaggtgaccgacgtggtggtggacgctacggacagcaggctgctgatgatgagcgtgtacgccctgagcgccattatcggaatatacctgctgtacaggatgttgaagacctgt

SEQ ID NO: 104 - DNA, 2226 bp, synthetic construct
  misc_feature 1..2226: note = Synthetic polynucleotide
  source 1..2226: mol_type = other DNA
atgcggccgggactccctagctacctcatcatcctcgccgtgtgccttttctctcaccttttgagctccagatacggcgccgaagcggtgtccgagcctctcgacaaggccttccacctccttctcaacacatacggcagaccgatccggttcttgagggagaacactacccaatgcacatataacagtagcctgcggaatagcactgtggtgcgggagaacgccatcagcttcaatttcttccagagctacaatcagtattacgtgttccacatgcctagatgtctgttcgcgggccctctggcagagcagttcctgaaccaggttgatctcacagagacactggagagataccagcaaagactgaacacctacgctctcgtctccaaggacctggccagctataggagcttcagccagcagctgaaggcccaggattccctgggagagcagccaaccaccgtcccgccgccaatcgacctctctatcccacacgtgtggatgcctccacaaaccacaccacacggatggactgagtcccataccaccagcggactgcacaggcctcacttcaatcagacctgcattttgttcgacggccacgacctcctgttcagcactgtgaccccgtgtctgcatcagggcttctacctgattgacgagctgaggtacgtcaagattacgctcaccgaagacttcttcgtggtcacagtgagtatcgatgacgacacccctatgctgctcatcttcggccatctgcctagggtgctgttcaaggccccttaccagagagataatttcatcttgcggcagactgagaagcacgaactgctggtactcgtgaagaaggaccagctgaaccgccactcttacttaaaggatccagacttcctggatgcagcacttgacttcaactatctcgacctctctgccctgctgaggaacagcttccaccggtatgccgtggacgtgctgaagagtggacggtgtcagatgctggaccgcagaacagtggaaatggcgttcgcgtatgctctggccctattcgcggccgcaagacaggaggaggccggcgctcaggtgtccgtccctagagctctggacaggcaggccgccctgctgcaaattcaggagttcatgataacttgcctgagccaaacccctccgagaacaacactgctgctgtatccaacagccgtagatctggccaagcgggccctttggactcctaaccagatcaccgatattacctccctggtgagactggtgtacattctgtccaagcagaaccagcagcacctgatcccgcagtgggccctgagacagatcgctgatttcgccttgaagctgcacaagactcatctggcctccttcctgagtgctttcgcccgccaggaactgtatctgatgggctctcttgtccattccatgctggttcataccacggagagaagggagatcttcatcgtggaaaccggcctttgctccctcgctgagctgagccatttcactcagctgctcgcccacccgcaccacgagtacctgtcagacctttatactccgtgctcctccagcggcaggagggaccacagcctggaacggctcacaagactgttcccggatgctaccgtgcctgctactgtgccagccgccctgagcatcctttccaccatgcagccttccacactggagactttccctgacctgttctgcctgccacttggcgaaagtttcagcgccctgaccgtgtccgaacatgtgagctacatcgtgactaaccagtacctgatcaagggcatcagctacccggttagcaccactgtcgtcggacagtcactgatcatcactcagaccgactcccagaccaagtgcgaactgaccagaaatatgcacacaacccatagcatcaccgtggccctgaacattagcctggagaactgtgccttctgccagagcgccctcctcgagtacgacgatacccagggtgtgataaacattatgtatatgcacgacagtgacgacgttctgttcgcactggacccttacaacgaagtggtcgtttcctctcctcggacccattacctgatgctgctgaagaacggcaccgtgctagaggttactgatgtggtagtggacgccacagacagcagactgctgatgatgagcgtgtacgccctgagcgccattattggcatctacctgctgtacaggatgctgaagacatgt

SEQ ID NO: 105 - DNA, 2226 bp, synthetic construct
  misc_feature 1..2226: note = Synthetic polynucleotide
  source 1..2226: mol_type = other DNA
atgcggccgggactcccttcctacctcatcatccttgccgtgtgtcttttctcacacctcctcagcagccgctacggcgccgaggccgtgtcagagccacttgacaaggccttccatcttttgttgaacacctacggcagacctattcggttcctgagagagaacacaacccagtgcacctataacagctctctgcgcaactccacagtggttagagagaatgccatcagcttcaacttcttccagagttacaaccagtattacgtgttccatatgcctaggtgcctgttcgctggcccgttagccgaacagttcctcaaccaggtggatctgaccgaaacactggaaaggtaccagcagcggctgaatacatacgccttggtgtcaaaggatcttgcttcctacagaagcttcagccagcagctgaaggcccaggacagccttggagagcagccaaccaccgtgcctcctcctattgacctgagcatccctcatgtgtggatgcctcctcagaccacccctcacggctggactgagagccataccacgtccggcctgcacaggcctcacttcaatcagacctgcatcctgttcgacggccacgatctgcttttcagcaccgtcaccccttgcctgcaccagggattctacctgatcgacgagctccggtatgtgaagattacactgaccgaggacttcttcgtggtgaccgtgtccatcgacgatgacaccccaatgctgctgatcttcggccacctgccgagagtcctgttcaaggccccataccagagagacaacttcatcctgcggcagaccgagaagcacgaactgctagtgctggtgaagaaggatcagctgaaccggcactcctacctgaaggaccctgacttccttgacgccgcactcgacttcaactacctggacctcagtgctctactgaggaactctttccaccggtacgccgtggacgtgctgaagtctggaagatgccagatgctggataggaggacagtggagatggcgttcgcgtacgccctggccctgttcgccgccgccagacaggaggaggccggcgcacaggtcagcgtcccaagggccctggaccgccaggctgccctgctgcagattcaggaattcatgatcacctgtctcagccagacccctccgagaacaaccctgctgttgtacccgaccgcagtggatctggctaagagggccctgtggaccccaaaccagattaccgacatcacctctctggtgagactggtgtacatcctgtccaagcagaaccaacagcacctcattccacaatgggccctgaggcaaatcgccgatttcgctctcaagttgcataagacccatctggcctcattcctcagcgccttcgcaagacaggagttgtatctcatgggctccctcgtgcatagcatgctggtgcacacaaccgagcgcagagaaattttcatcgttgaaaccggactgtgcagcctcgccgagttgtctcatttcacccagctgctggctcatcctcaccatgagtatctttccgacctgtacaccccgtgcagcagcagcggccgcagggatcacagcctcgagagactgacaagactgttcccagacgccaccgtgcctgccacagtgccagccgcgctgtccatcctgagcacaatgcagcctagcacactggagactttcccagatctgttctgtcttccactgggcgagagcttcagcgccctgaccgtgagcgagcacgtgagctacatagtgaccaaccaatatttgattaagggcatctcctaccctgtgagcaccacagtggtgggccagtctctgatcatcacacaaaccgacagtcagacgaagtgcgagctgactagaaacatgcacacgacccacagcataaccgtggcactcaacatctccctggagaattgcgccttctgccagagcgccctcctggagtacgacgacactcaaggagtgatcaacatcatgtacatgcacgatagcgatgacgtgctgttcgccctggacccatacaatgaagtggtggtgtccagcccacggacccactacctgatgctcctcaagaacggcacagtgctggaggttacagacgtggtggtcgacgctaccgatagcagacttcttatgatgtccgtgtacgccctgagcgccatcatcggaatctatctgctttacaggatgctgaagacttgc

SEQ ID NO: 106 - DNA, 2226 bp, synthetic construct
  misc_feature 1..2226: note = Synthetic polynucleotide
  source 1..2226: mol_type = other DNA
atgcggccaggcctcccttcgtaccttatcatcttggccgtgtgtcttttctctcacctactctcctcaaggtacggcgccgaggccgtgagtgagccgctagacaaggccttccacctattacttaacacctacggccggcctatccgattcctccgggagaataccacacagtgtacatacaactctagcctgcgcaacagcactgtggtcagggagaacgccatcagcttcaatttcttccagagttataaccagtactacgtgttccacatgccaagatgcctgttcgccggacctcttgccgagcagttcctgaaccaggtggatctgaccgagacactggagagataccagcaaaggctcaatacctacgctttagtgagcaaggaccttgcttcttacagatctttctcacaacagcttaaggcgcaggacagcctcggcgagcagcctaccaccgtgcctcctcctattgacttgagcatcccacacgtatggatgcctccacagacgacaccacacggctggaccgaatcccatacaaccagcggactccaccggcctcatttcaatcagacctgcatccttttcgacggccatgacctgctattctctaccgtcaccccgtgcctgcaccagggcttctaccttatcgacgaactgagatacgtcaagatcacgctgaccgaagacttcttcgtcgttacagtcagcatcgacgacgatacccctatgctgctgatattcggccaccttcctagagtcctgttcaaggcaccgtaccagagggacaacttcatcctgagacagacagagaagcacgagctcctggtgctggtgaagaaggatcagctgaatagacacagttacctgaaggatccagacttcctggacgccgcactggacttcaactatctggatctgagcgccctgcttcgcaatagcttccatagatacgctgtggacgtgctgaagtctggccggtgtcagatgctggatcgtaggaccgtggagatggccttcgcctacgcactcgctctgttcgccgccgctagacaggaggaggccggagcccaagtgagtgtgcctcgggcactggacagacaggcagccttactgcagatccaggagttcatgattacctgcctgtctcagactccaccacggaccacccttctgctgtatcctaccgcggttgatctggctaagagggccctgtggacccctaaccagatcactgacatcaccagcctcgtgaggctggtgtacattcttagcaagcagaaccagcagcacctaatacctcagtgggccctgcggcagatcgccgacttcgccctgaagctgcacaagacccacctggcaagcttcctgtccgccttcgcccgccaggagctgtacctcatgggaagtctggtacactccatgctggtgcacaccaccgagagaagagagatcttcatcgtagaaaccggactctgctcactggccgaattgtcacacttcacccagctgctggcccatcctcatcacgagtatctgtccgacctgtacaccccttgcagctctagcggcaggcgggaccattccttggagaggctgaccaggctgttcccggatgccaccgttccagcaacagtgcctgcagccctgagcattctgtcaacaatgcagcctagcaccctcgaaactttcccggatctgttctgcctgcctcttggtgagagcttcagcgccctgaccgtgtccgagcatgtgtcttatatcgtaaccaatcagtacctgatcaagggcatcagctaccctgtgtccacaactgtcgtgggccaaagcctcatcataacccagaccgattcccagacaaagtgtgaactgacccgcaacatgcacaccactcacagcattactgtggccctgaacatctccctggagaactgtgccttctgtcagagcgcgcttctggagtatgatgacacccagggtgtgattaatatcatgtacatgcacgacagcgacgatgtgctgttcgcgctggatccttacaatgaggtggtcgtgagctcccctagaacccactatctcatgctgttaaagaacggcaccgtcctggaggtgacagacgtggtggttgatgccaccgacagcaggctgctgatgatgagcgtttatgccctgagcgccatcatcggcatttacctcctgtacaggatgttaaagacttgt

SEQ ID NO: 107 - DNA, 2226 bp, synthetic construct
  misc_feature 1..2226: note = Synthetic polynucleotide
  source 1..2226: mol_type = other DNA
atgcggccaggactccctagctacctcattatcctcgccgtgtgccttttctctcacctcttaagctcccggtacggcgccgaagccgtgagcgagccgcttgacaaggctttccacctcctccttaacacctacggcagacctattagattcctgagagagaacaccacccagtgtacatataattctagcctgcggaactccaccgtagtgagggagaatgccattagcttcaacttcttccagagctacaaccagtactatgtgttccatatgccgagatgtctgttcgctggacctctcgcagaacagttcttgaaccaggtggatctgactgaaactctcgagcggtaccagcaaagactgaatacctatgccttggtaagcaaggatctggctagctacaggagcttctcccagcagctcaaggcccaggactcccttggcgaacagcctaccaccgtccctcctccaattgacctgagcattccgcacgtgtggatgcctcctcagaccaccccacacggctggacagagtctcataccaccagcggactgcatagaccgcatttcaaccagacttgcatcctgttcgatggacatgatctcctgttctctacagtgactccatgcctgcaccagggcttctacctgatcgatgagctcagatacgtcaagatcaccttgaccgaagatttcttcgtggtcacagtgagcattgacgacgacaccccaatgcttctgatattcggtcacctgcctagggtcctcttcaaggctccataccagagagacaatttcatccttagacagaccgagaagcacgagctgctcgtgctggtgaagaaggatcaactgaacagacatagctacctaaaggatccggatttcctggacgccgctctggacttcaactacctcgacctcagcgccctgctgaggaacagcttccaccggtatgcagtcgatgttctcaagtccggcagatgccagatgctggaccgtagaactgtggagatggccttcgcctatgctctggccctgttcgccgccgcacgccaggaagaggctggagcccaggtgagcgtcccacgggctctggacagacaggctgctctgctgcagatccaagagttcatgattacctgtctgagccagacccctcctagaaccaccctcctcctctatccgaccgctgtggacctggccaagagagccttgtggacccctaatcagattactgacatcacaagcctggtcagactggtgtatatcctgagcaagcagaatcagcagcacctcattccacagtgggcgctgcggcagatcgctgatttcgccctgaagctgcacaagacccacctggccagcttcttgagcgcattcgcacggcaggaactctacctgatgggctctctggtgcacagcatgctcgtccacaccacagaacggcgagagatattcatcgttgagacaggcctgtgctctctggccgagttgtcccacttcacccaactgctggctcaccctcatcacgagtacctcagcgacctgtacaccccttgctcctccagcggtagacgggatcacagcctggaaagactgaccagactgttcccagacgccacggtccctgcaaccgtgcctgccgctctttcaatcttgtccaccatgcagcctagtacactggaaacattccctgacctcttctgcctgcctctcggagagtccttctcagccctgaccgtgagcgaacacgtgtcctacatcgtgaccaaccagtacctgatcaagggcatctcctaccctgtgtcgaccaccgtcgtgggccagagcctgatcattacacagacggactctcagaccaagtgcgagttgacacggaacatgcacaccacacacagcatcactgtggccctgaatattagcctcgagaactgcgccttctgccagagtgccctgctagagtatgatgatacacagggcgtgattaatatcatgtatatgcacgactctgatgacgtcctgttcgccctggacccatacaacgaggtggttgtgagctcccctcggacccactatctgatgctgctcaagaacggcactgttctcgaggtgacagatgtggtggtcgatgccacagattctcggctgctgatgatgagcgtgtacgctcttagcgccatcatcggaatctacctcctgtacaggatgctgaagacttgt

SEQ ID NO: 108 - DNA, 2226 bp, synthetic construct
  misc_feature 1..2226: note = Synthetic polynucleotide
  source 1..2226: mol_type = other DNA
atgcggccgggcctcccgtcttacctcatcatcttggccgtgtgcctcttctcccacctcttgagctcccggtacggcgcagaggccgtgtcagagcctctcgacaaggccttccatttgctccttaacacatacggcaggccaatcaggttcctgcgagagaataccacacaatgcacctacaactccagcttgaggaatagcaccgtggtgcgggagaacgccatctccttcaatttcttccagtcctacaatcagtattatgtgttccatatgcctaggtgtctcttcgcaggcccacttgccgaacaattcctgaaccaagtggacctgacagagacactggagagataccagcaacggctgaatacctacgccttggtgagcaaggatctcgccagctacagatctttctcacaacaactgaaggcccaggattctctcggtgagcagccaacgactgtgcctcctccaattgacctgtctatcccacatgtgtggatgccacctcagactacccctcacggatggacagagtctcataccactagcggcctgcacaggcctcacttcaatcagacctgtatcctcttcgacggtcacgatctgttgttcagcaccgtgaccccatgcctgcatcagggcttctacctgattgacgagctgagatatgtgaagataacactgaccgaggatttcttcgtggtcaccgtgagcatagacgacgacacaccgatgctcctgatcttcggccatctgccacgagttctgttcaaggcaccttatcagagagacaacttcatcttgaggcaaacagagaagcacgagcttctcgtgctggttaagaaggaccagctcaacaggcatagctacctgaaggacccagatttcctggacgccgctctggatttcaattatctggacctttctgctctgctgagaaacagcttccatagatacgccgtggacgtccttaagtctggccgctgccagatgctggatagacggactgtcgagatggcattcgcctacgctctggctctgttcgccgccgccaggcaggaggaggctggagcccaagtgtcagtgcctagggctctggatagacaagccgccttgctccagatccaggagttcatgattacctgtctgagccagaccccaccaagaaccacgttactgctgtaccctaccgctgtggacctggctaagcgagccctctggacgcctaatcaaatcaccgacatcaccagcttagtcagactggtgtacattctgtctaagcagaaccagcagcacttgattccacagtgggccctgagacagattgccgacttcgccctgaagctccataagacccatctggcgtccttcctgagcgccttcgccagacaggagctctacctgatgggcagcctggttcattccatgctggtccatacaacggagagaagagagatcttcatcgtggagacaggactgtgctctttggccgaactttcccacttcactcagctgctggcgcaccctcatcacgagtacttatcggacctgtacaccccttgcagcagcagcggaaggagggaccattctctcgaaaggctgacaagactgttccctgacgccaccgtcccagccacagtgcctgccgcactgagcatcctcagcacaatgcagccaagcactctggagactttcccggacttgttctgcctgccgctgggcgagtccttcagcgcccttacagtgtcagagcatgtgtcctacatcgtgaccaatcagtacctgatcaagggaatcagctaccctgtgtctacaaccgtggttggccagtccctcatcatcacccagacagatagccaaactaagtgcgaactgactagaaacatgcacacaacccactccatcacagtggccctgaacatcagcctcgagaattgcgccttctgccagagcgcactgttggagtacgacgatactcagggcgtgattaacatcatgtacatgcatgatagcgacgatgtgctgttcgccctggacccttataacgaggtggtggtgagtagtcctaggacccattaccttatgctgctgaagaacggaactgttctggaggttaccgacgtcgtcgttgacgctaccgactcacgcctgctcatgatgtctgtgtatgccctgtctgccatcatcggcatctacctgctgtataggatgctgaagacttgc

SEQ ID NO: 109 - DNA, 2226 bp, synthetic construct
  misc_feature 1..2226: note = Synthetic polynucleotide
  source 1..2226: mol_type = other DNA
atgcggcccggccttcccagctacctcatcatcctcgccgtgtgcttgttcagccaccttctaagcagccggtacggcgccgaggccgtgagcgagcccctcgacaaggccttccacttgttattgaacacctacggccggcccatccggttcctgcgggagaacaccacccagtgcacctacaacagcagcctgcggaacagcaccgtggttcgggagaatgccatcagcttcaacttcttccagagctacaaccagtactacgtgttccacatgccccggtgtcttttcgccggaccgctggccgagcagttcctgaaccaggtggacctgaccgaaactctggagcggtaccagcagcggctgaatacctatgccctggtgagcaaggacctggcctcataccggagcttcagccagcagctgaaggcccaggacagcctgggcgagcagcccaccaccgtgcctccacccatcgacctgagcatcccgcacgtgtggatgcctccacagaccacacctcacggctggaccgagagccacaccaccagcggcctgcaccggccccacttcaaccagacctgcatcctgttcgacggccacgacctgctgttcagcacagttaccccttgccttcatcagggcttctatcttatagacgagctgcggtacgtgaagatcacactcaccgaggacttcttcgtggtgaccgtgagcatcgacgacgacactcctatgctcctcatcttcggccatcttccccgggttctgttcaaggctccataccagcgggacaacttcatcctgcggcagaccgagaagcacgagttgctggtgctggtgaagaaggaccagctgaaccggcattcgtaccttaaggaccccgacttcctggacgccgccctggacttcaactacctagatctcagcgccctgttgaggaatagcttccaccggtacgccgtggacgtgttaaagagcggccggtgccagatgctggatcggcggaccgtggagatggccttcgcctacgcgctggccttgttcgctgccgcccggcaggaggaggccggcgcccaggtgagcgtaccgcgggcactcgatcggcaggccgctctgctgcagatccaggagttcatgatcacctgcctgagccagacccctccgcggaccaccttactgctttaccccactgcagttgacctggctaagcgcgcactctggacccctaaccagatcaccgacatcaccagcctggtgcggctggtgtacatcctgagcaagcagaaccagcaacacctgataccacagtgggctctgagacagatcgccgacttcgccctgaagctgcacaagacccatctggccagcttcctgtccgctttcgcacgacaggagctgtacctgatgggatcactcgtgcacagcatgctcgtgcataccaccgagcggcgggagatcttcatcgtggaaacaggcctgtgttcactagccgaactgagccacttcacccaacttttggcccatccacaccacgagtatttgtcggacctgtacaccccttgttcctcttccggaaggcgggaccactccctggaacggctgacccggctgttccccgacgcaaccgtaccggccacggttccagctgccttaagcatcttaagtaccatgcagcccagcacactggaaaccttcccagatctgttctgcctgccgctgggtgagtctttcagcgctctcaccgtgtccgagcacgtgagctacatcgtgacaaatcaatatctgattaagggcatcagctacccagtgtcaactacggtggttggccagagcttgattataacccagaccgactcgcagactaagtgcgagcttacgagaaacatgcacacaacccacagcatcaccgtggccctgaacataagtctggagaactgcgccttctgccagtctgccttgctcgagtatgatgacacccagggcgtgatcaacatcatgtacatgcatgacagcgacgatgttctcttcgcgttggatccatacaacgaggtggtggtgtccagtccgagaactcactatctgatgctcctaaagaacggcaccgtgctggaggtgaccgacgtcgtggtcgatgccacggactccagactgcttatgatgagcgtgtacgccctaagcgccatcatcggcatctatctcctgtatcggatgcttaagacctgc

SEQ ID NO: 110 - DNA, 2226 bp, synthetic construct
  misc_feature 1..2226: note = Synthetic polynucleotide
  source 1..2226: mol_type = other DNA
atgcggcccggccttcccagctacctcatcatcctcgccgtgtgcttgttcagccacttgcttagcagccggtacggcgccgaggccgtgagcgagcccttggacaaggccttccacttactcctcaacacctacggccggcccatccggttcctgcgggagaacaccacccagtgcacctacaacagcagcctgcggaacagcaccgtggtgcgtgagaacgccatcagcttcaacttcttccagagctacaaccagtactacgtgttccacatgcccaggtgccttttcgccggaccactggccgagcagttcctgaaccaggtggacctgaccgaaacactggagcggtaccagcagaggctgaacacatacgccctggtgagcaaggacctggcctcctaccggagcttcagccagcagctgaaggcccaggacagcctgggcgagcagcccaccaccgtgccgccacccatcgacctgagcatcccacacgtgtggatgccaccacagaccacccctcacggctggaccgagagccacaccaccagcggcctgcaccggccccacttcaaccagacctgcatcctgttcgacggccacgacctgctgttctcgaccgtcaccccttgcttgcaccagggcttctatctgatagacgagctgcggtacgtgaagatcaccttgaccgaggacttcttcgtggtgaccgtgagcatcgacgacgacacacctatgctgctcatcttcggccatttaccccgggttctgttcaaggcaccataccagcgggacaacttcatcctgcggcagaccgagaagcatgagcttctggtgctggtgaagaaggaccagctgaaccggcactcatatctgaaggaccccgacttcctggacgccgccctggacttcaactacctggatttaagcgccctgctccgtaactctttccaccggtacgccgtggacgtgttaaagtcaggccggtgccagatgttggaccggcggaccgtggagatggccttcgcttacgcattagccctcttcgcagccgcccggcaggaggaggccggcgcgcaggtgagcgtgcctagagcgttggatagacaggcggccttgctgcagatccaggagttcatgatcacctgcctgagccagactcctccacggaccacattgctgctctaccccaccgccgttgacctggcaaagcgggcgctctggactccgaaccagatcaccgacatcaccagcctggtgcggctggtgtacatcctgagcaagcagaatcagcagcacctgataccacagtgggcactacgccagatcgccgacttcgccctgaagctgcacaagacccacctggccagcttcctgtctgctttcgcaaggcaggaactgtacctgatgggctctctagtgcacagcatgctcgtccataccacagagcggcgggagatcttcatcgtggagactggcctgtgctctcttgcggaactgagccacttcacccagctcctagcccacccacaccacgagtacctttctgacctgtacaccccgtgctcatcaagtggacggcgggaccactcgctggaaagactcacccggctgttccccgacgctactgtgccggcaactgtgcctgcggctctctctatattatctaccatgcagcccagcacactcgaaaccttccctgatctgttctgcctgcctctaggagagagcttctctgcccttacagtgtccgagcacgtgagctacatcgtgacaaaccaatacctcattaagggcatcagctaccctgttagtactaccgtcgtaggccagagcctaattatcacccagaccgactcccagacaaagtgcgaattaacgcgcaacatgcacacaacccacagcatcaccgtggccctgaacattagcctcgagaactgcgccttctgccagagtgccttgcttgagtatgatgatacccagggcgtgatcaacatcatgtacatgcacgacagcgacgatgtgctgttcgcactcgacccctacaacgaggtggtcgtaagcagtccaaggacccattatttgatgctgcttaagaacggcaccgtgctggaggtgaccgacgtggtggtagacgctacagactcccggctgcttatgatgagcgtgtacgcgctcagtgcgatcatcggcatctacctgctttatcggatgctaaagacctgc

SEQ ID NO: 111 - DNA, 2226 bp, synthetic construct
  misc_feature 1..2226: note = Synthetic polynucleotide
  source 1..2226: mol_type = other DNA
atgcggcccggcttgcccagctacttgatcatcttggccgtgtgcttgttcagccacttacttagcagccggtacggcgccgaggccgtgtccgagcccctcgacaaggccttccacctcctcctcaacacctacggccgccccatccgcttcctccgcgagaacaccacccagtgcacctacaactcctccctccgcaactccaccgtcgtgcgggagaatgccatctccttcaacttcttccagtcctacaaccagtactacgtcttccacatgccccgctgcctcttcgccggacctctcgccgagcagttcctcaaccaggtcgacctcaccgagacgctcgagcgctaccagcagaggttgaatacctatgccctcgtctccaaggacctcgcctcctaccgctccttctcccagcagctcaaggcccaggactccctcggcgagcagcccaccaccgtgcctccaccaatcgacctctccatcccgcacgtctggatgcctccccagaccactccgcacggctggaccgagtcccacaccacctccggactgcatcgccctcacttcaaccagacctgcatcctcttcgacggccacgacctcctcttcagtaccgtgacgccatgcctgcaccagggcttctacctcatcgacgagctccgctacgtcaagatcacccttaccgaggacttcttcgtcgtcaccgtctcgattgacgacgacacaccaatgctcctcatcttcggccacctcccgcgcgtgctgttcaaggcgccctaccagcgcgacaacttcatcctgaggcagaccgagaagcacgagctgctcgtcctcgtcaagaaggaccagctcaaccgccactcctacctcaaggaccccgacttcctcgacgccgccctcgacttcaactacctcgatctgagtgctctgctgaggaattcattccaccgctacgccgtcgacgtcctcaagtccggccgctgccagatgctcgaccgccgcaccgtcgagatggccttcgcttacgcgctggcactcttcgctgccgcccgccaggaggaggccggcgcccaggtcagtgtgccaagagcactggatagacaggccgcacttttgcagatccaggagttcatgatcacctgcctctcccagactccgcctcgcaccacgttgttgttgtaccccacagcagtcgatctggctaagagagccttatggacacctaaccagatcaccgacatcacctccctcgttcggctggtctacatcctctccaagcagaatcagcagcacctgatccctcagtgggcactccgtcaaatcgccgacttcgccctcaagctccacaagacccacctggcgtctttcctcagtgcattcgctaggcaggagctgtacctgatgggcagtctcgtccactccatgctggtgcacaccacggagcgccgcgagatcttcatcgtcgagactggcctctgtagtttagccgagctcagtcacttcacccaactgctcgcccaccctcaccacgagtaccttagtgacctctataccccttgctctagttccggtcgccgcgaccacagcctggaacgtctgacccgcctcttccccgacgctacagtaccagcaaccgtgccagcggccctgtctatcctgtctaccatgcagccctccaccttggagacgttcccagatctgttctgcctccctctaggcgaatcgttctctgcactcacggtcagcgaacacgtctcctacatcgtcacaaaccagtatctgataaagggcatctcctacccagtgtcgaccactgttgtcggccagtccctcatcatcacacagactgattctcagactaagtgcgagttgacacggaacatgcatactacacattctatcaccgttgctttaaacataagcctggagaactgcgccttctgccagtccgctctgctcgagtacgacgatacgcagggcgtcatcaacatcatgtacatgcacgactccgatgacgttctgttcgcactggacccctacaacgaggtcgtcgtctcctctcctaggactcactacttaatgctgttgaagaacggcaccgtcctcgaggtcaccgacgtggtcgtcgatgctacagacagccgactgctcatgatgtccgtgtacgctctctccgccatcatcggcatctacctgctctaccgcatgctcaagacctgc

SEQ ID NO: 112 - DNA, 2226 bp, synthetic construct
  misc_feature 1..2226: note = Synthetic polynucleotide
  source 1..2226: mol_type = other DNA
atgcggcccggcctacccagctaccttatcatcttagccgtgtgcttgttcagccacttgctcagcagccggtacggcgccgaggccgtgtccgagcccctcgacaaggccttccacctcctcctcaacacctacggccgccccatccgcttcctccgcgagaacaccacccagtgcacctacaactcctccctccgcaactccaccgtcgtcagagagaatgccatctccttcaacttcttccagtcctacaaccagtactacgtcttccacatgccccgctgcctcttcgccggcccactcgccgagcagttcctcaaccaggtcgacctcaccgaaactctcgagcgctaccagcagcggctgaatacgtatgccctcgtctccaaggacctcgcctcctaccgctccttctcccagcagctcaaggcccaggactccctcggcgagcagcccaccaccgtccctcctccaatcgacctctccatcccacacgtctggatgcctccccagaccactcctcacggctggaccgagtcccacaccacctccggcctccacaggccacacttcaaccagacctgcatcctcttcgacggccacgacctcctcttcagcacagtgacgccatgtctgcatcagggcttctacctcatcgacgagctccgctacgtcaagatcactctgacggaggacttcttcgtcgtcaccgtctctatagatgacgacaccccaatgctcctcatcttcggccacttgcctcgcgtgctgttcaaggctccctaccagcgcgacaacttcatcctccgccagaccgagaagcatgaattactcgtcctcgtcaagaaggaccagctcaaccgccactcctacctcaaggaccccgacttcctcgacgccgccctcgacttcaactacctggatcttagcgccctgctgcgtaacagcttccaccgctacgccgtcgacgtcctcaagtccggccgctgccagatgctcgaccgccgcaccgtcgagatggccttcgcctatgctctggccctgttcgctgccgcccgccaggaggaggccggcgcccaggtttccgttcctcgggctttagacagacaggctgccctgcttcagatccaggagttcatgatcacctgcctctcccagacgccaccacgcaccacactgttgctgtacccgacagcagtggacctggcaaagagggcactctggactccaaaccagatcaccgacatcacctccctcgtccgcctcgtctacatcctctccaagcagaaccagcaacacctgatacctcagtgggctctgcggcagatcgccgacttcgccctcaagctccacaagacccatttggcctccttcctgagtgccttcgctagacaggagctgtacctgatgggctccctggtgcactccatgctggttcacaccacagagcgccgcgagatcttcatcgtcgagacaggcctctgttcattggcagaactctcgcatttcacccagctgctggcccacccgcaccacgagtatctgagtgacctctacacaccatgcagttcgtcaggaaggcgcgaccacagcctggagcgactgacccgcctcttccccgacgcaacagtccctgctactgtccctgccgcgctgagtattttatcaacgatgcagccctccacgctcgaaaccttccctgacttgttctgcctccctcttggagagagtttcagtgccctgaccgtgtcagagcacgtctcctacatcgtcacgaaccagtatcttatcaagggcatctcctacccagtatcgactacagtggttggccagtccctcatcatcacccagactgacagtcaaactaagtgcgagcttactagaaacatgcacacaacccactccatcaccgttgcattaaacatcagcctcgagaactgcgccttctgccagagcgctctgctggagtacgacgacacacagggcgtcatcaacatcatgtacatgcacgactccgacgacgtcctgttcgcgcttgatccctacaacgaggtcgtcgtgtccagtccacgaactcattacctcatgctgcttaagaacggcaccgtcctcgaggtcaccgacgtggtcgtcgatgctaccgactcccgcctcctcatgatgtccgtttacgcactctctgcgatcatcggcatctacttactatatcgcatgctcaagacctgc

SEQ ID NO: 113 - DNA, 834 bp, synthetic construct
  misc_feature 1..834: note = Synthetic polynucleotide
  source 1..834: mol_type = other DNA
atgtgcagaagaccagactgcggattcagcttctccccaggcccagtgatcttactttggtgctgcctcctccttccgattgtgagcagtgccgccgtgagcgtggctcctaccgcggccgagaaggtgccggccgagtgcccagagctcacccgaaggtgccttctgggcgaggttttcgagggagacaagtacgagtcttggctccggcctctggtcaacgtgacgggcagagacggccctttgagccagctcatcagatacagaccagtaacacctgaggccgccaattccgtcctgctggatgaggccttcctggacaccctcgccctcctttacaataacccagaccagctgagagccctgttgacccttctcagcagcgacaccgctccacggtggatgaccgtcatgagaggctatagcgagtgcggagatggcagccctgccgtctatacctgcgttgacgacctgtgcaggggctacgatttgacaaggctcagctacggcagatctatattcacagagcatgtgctgggcttcgagctggtgccgccatccctgttcaacgtggtggtcgctataaggaacgaggccaccagaacaaatcgcgccgtgagactgccggtgtccacggcagccgcacctgagggcattacactgttctatggcctctacaacgccgtgaaggagttctgtttgcggcaccagctggacccaccactgctcagacacctggataagtactacgctggcctgcctccggagctgaagcaaacacgtgtgaatctgccagcccactctcggtacggaccgcaggccgtggacgcccgg

SEQ ID NO: 114 - DNA, 834 bp, synthetic construct
  misc_feature 1..834: note = Synthetic polynucleotide
  source 1..834: mol_type = other DNA
atgtgcaggaggccagactgcggcttctcattcagcccaggcccagtcatcctcctttggtgttgcctccttctccctatagttagcagtgccgccgtgagcgtggcccctacagccgcggagaaggtgccagcggagtgcccggagttaaccagacgttgcctcttgggcgaggtgttcgagggcgataagtatgagtcctggctgcggcctctggtgaacgtgaccggcagagacggacctctgtcccagctgatcagatacagaccagtgacccctgaagccgcaaacagcgtgctgctggacgaggccttcctggacaccctggccctgttatacaacaaccctgaccagcttcgcgcgctgcttacactgctgagcagcgataccgccccaagatggatgactgtgatgaggggatatagcgagtgtggcgacggcagccctgccgtctacacctgtgtggacgacctctgcagaggctatgacctgaccagactgtcatacggccgaagcatcttcaccgagcacgtcttaggattcgagctggtgcctccaagcctcttcaatgtggtggtggccattcggaacgaggctaccagaaccaaccgggcggtgcgtcttccagtttctacagccgccgccccggaaggaattaccctgttctacggcctgtacaacgctgtcaaggagttctgcctgagacaccagctggatccaccgctgctgcgccacttggacaagtactatgcgggcctccctcctgagctcaagcagacgagggtgaacctccctgctcactcacgttatggaccacaggccgtggacgctaga

SEQ ID NO: 115 - DNA, 834 bp, synthetic construct
  misc_feature 1..834: note = Synthetic polynucleotide
  source 1..834: mol_type = other DNA
atgtgcagaaggccagactgcggcttcagcttctctccaggaccagtgatcctcctctggtgctgccttctcctccctattgtgtcctccgccgccgtgtccgtggccccaaccgccgccgagaaggtgccagcggagtgcccagagctcaccaggcgctgtctgctgggcgaggtgttcgagggcgataagtacgagagttggctgaggccgttggtgaacgtgacgggcagggacggcccgctaagtcagttaataaggtaccggccagtgaccccggaggccgccaacagcgtgctgctggatgaggccttcttggacaccctggccctgttgtacaacaacccagaccagctgagagccctgctgactctgttgagcagcgacaccgccccaagatggatgaccgtgatgagaggctatagcgagtgcggcgatggcagccctgccgtgtacacgtgcgtggacgatttgtgtagaggctacgacctcaccagactgagctacggcagaagcatcttcactgagcatgtgctgggattcgagctggtgcctcctagcctgttcaatgttgtggtggctatacgcaacgaggccacaagaacaaacagggccgtaagactcccagtgagcaccgctgcagcccctgagggaatcacgctgttctacggcctctacaacgctgtgaaggagttctgtctgaggcaccaactggacccacctctgcttagacacctggataagtactacgccggcctcccacctgaactgaagcagaccagggtgaatcttcctgcacactcaaggtatggcccacaggccgtggatgccagg

SEQ ID NO: 116 - DNA, 834 bp, synthetic construct
  misc_feature 1..834: note = Synthetic polynucleotide
  source 1..834: mol_type = other DNA
atgtgcaggcggccagactgcggcttcagcttctcaccgggcccagtcatcttgttgtggtgctgcctcctcctccctatcgtaagctcggcagccgtcagtgtggccccaaccgccgccgagaaggttccagccgagtgtccagaactcacaaggcggtgcctgctgggcgaggtcttcgagggcgacaagtatgaaagctggctcaggccacttgtcaatgtgacaggcagagacggcccactgagccagcttatccggtatagacctgtcactcctgaggccgctaacagcgtgcttctggacgaggctttcctggacactctggctctgctttacaacaacccagaccagctgagagccctgctgaccctgctgagcagcgatacagccccaaggtggatgacagttatgaggggatacagcgagtgtggcgacggaagcccagccgtgtatacctgcgtggatgacctgtgcagaggctacgacctgacccgcctctcctacggaagatccatcttcaccgagcacgtgctaggattcgagctcgtccctcctagcctgttcaatgtggtggtggccatcagaaacgaggccactcggaccaatagagcagtgagactgccagtgagcaccgcggccgcaccagagggtatcacactgttctacggcctgtacaacgccgtgaaggagttctgtctgcgtcaccagctggacccacctctgcttagacatctggataagtactatgccggcctgcctcctgaactcaagcagacccgtgtgaatctgcctgcccactccagatacggccctcaggccgtggacgcaagg

SEQ ID NO: 117 - DNA, 834 bp, synthetic construct
  misc_feature 1..834: note = Synthetic polynucleotide
  source 1..834: mol_type = other DNA
atgtgtagacgaccagactgcggcttctctttctctccaggcccggtgatcttactctggtgttgtttgctccttcctatcgttagtagcgccgccgtgagcgtggctccgacagccgccgagaaggtgccagccgagtgcccagagctcaccagaagatgtctgctgggcgaggtcttcgaaggcgacaagtacgagtcttggctgaggcctctcgtgaatgttaccggcagggacggcccactgagccagctgattaggtaccggccagtgaccccggaagccgccaacagcgtgctgctggatgaagccttcctggacaccctggccctgctgtacaacaatcctgaccagctccgggccctgctgaccctcctcagcagcgacactgcccctcggtggatgacagtcatgagaggctactccgaatgtggagacggcagccctgccgtctacacctgtgtggacgacctctgtaggggctacgacctgacaagactgtcctatggcagaagcattttcaccgagcatgtgcttggcttcgagctggtgcctccatccctgttcaacgtggttgtggccattagaaacgaggccaccagaaccaacagggccgtgcggctgccagtgagtacagccgctgctccagagggcattaccctgttctacggcctttacaatgccgtgaaggagttctgtctgcgccatcagctggaccctcctctgctgagacacctggataagtattacgcgggcctgcctccagaactgaagcagacccgcgtcaacctgccagctcatagccgttacggcccgcaagcagtggacgcccga

SEQ ID NO: 118 - DNA, 834 bp, synthetic construct
  misc_feature 1..834: note = Synthetic polynucleotide
  source 1..834: mol_type = other DNA
atgtgccgaagaccggactgcggcttcagcttcagcccaggcccggttatcctcctctggtgctgcctccttctcccaatcgtgagcagcgccgccgtgagcgtggcgcctaccgccgcggagaaggtcccagccgagtgcccagaattgacgaggagatgcttgctgggcgaagtgttcgagggcgataagtatgagagctggctgcggcctctggtcaacgtgaccggccgcgacggcccactgtcccagctgatcaggtacagaccagtgaccccagaggctgctaacagcgtgctgctggatgaggctttcctcgacacgctggctctcctgtacaacaatccggatcagctcagagccctgctcacactgctgtccagcgacaccgctccaaggtggatgacagtgatgcggggctactcagagtgcggcgacggtagccctgcggtgtatacatgtgtggacgacctgtgcagaggctacgacttaaccaggctgtcctacggtagatccatcttcactgagcacgtgctgggcttcgagctggtgccacctagcctgttcaatgtcgtggtagccatccggaacgaggctaccagaacaaatcgggccgtgaggctcccagtgagcaccgccgccgctcctgagggcatcactctgttctacggactttacaacgccgtcaaggagttctgcctgcggcaccagctcgatccacctctgctgagacacctggacaagtactacgccggccttccgcctgagctgaagcagaccagagtcaacctgcctgcccatagcagatacggcccacaggctgtggatgccaga

SEQ ID NO: 119 - DNA, 834 bp, synthetic construct
  misc_feature 1..834: note = Synthetic polynucleotide
  source 1..834: mol_type = other DNA
atgtgccggcggcccgactgcggcttcagcttcagccccggccccgtgatcctattgtggtgctgcctacttttgcccatcgtgagcagcgccgccgtgagcgtggctcctaccgccgccgagaaggtgcccgccgagtgccccgagctaacccggcggtgccttcttggcgaggtgttcgagggcgacaagtacgagagctggctgcggcccctggtgaacgtgaccggccgggacggccctctgagccagctgatccggtaccggcccgtgacaccagaggccgccaacagcgtgctgctggacgaggccttcctggacaccctggccctgctgtacaacaaccccgaccagctgagagctctgctgacgctgctgtcaagcgacaccgcgccacggtggatgaccgtgatgcggggctacagcgagtgcggcgacggcagccccgccgtgtacacctgcgtggacgacctgtgcaggggctatgacctgacccgtctgagctacggccggagcatcttcaccgagcacgtgctgggcttcgagctggtgcctcccagcctgttcaacgtggtggtggccatccggaacgaggccacccggaccaaccgggccgtgcggctgcccgtgagcaccgcagcagccccagaaggcatcaccctgttctacggcctgtacaatgccgtgaaggagttctgcctgcggcaccagctggacccacctcttctcaggcacctggataagtactacgccggcctgcctcctgaactgaagcagacccgggtgaacctgcccgcccacagccggtatggcccacaggccgtggacgcccgg

SEQ ID NO: 120 - DNA, 834 bp, synthetic construct
  misc_feature 1..834: note = Synthetic polynucleotide
  source 1..834: mol_type = other DNA
atgtgccggcggcccgactgcggcttcagcttcagccccggccccgtgatcctcctctggtgctgcctcttgttgcccatcgtgagcagcgccgccgtgagcgtggctcctaccgccgccgagaaggtgcccgccgagtgccccgagctcacccggcggtgccttctgggcgaggtgttcgagggcgacaagtacgagagctggctgcggcccctggtgaacgtgaccggccgggacggccctctgagccagctgatccggtaccggcccgtgactccagaggccgccaacagcgtgctgctggacgaggccttcctggacaccctggccctgctgtacaacaaccccgaccagctgagggcccttctgaccctgctcagcagcgacaccgccccacggtggatgaccgtgatgcggggctacagcgagtgcggcgacggcagccccgccgtgtacacctgcgtggacgacctgtgcaggggctacgacctgaccaggctgagctacggccggagcatcttcaccgagcacgtgctgggcttcgagctggtgccgcccagcctgttcaacgtggtggtggccatccggaacgaggccacccggaccaaccgggccgtgcggctgcccgtgtcgacagccgccgcccctgagggcatcaccctgttctacggcctatataacgccgtgaaggagttctgcctgcggcaccagctggacccgcccctgcttcgccacctggacaagtattacgccggcctgcctccggagctgaagcagacccgggtgaacctgcccgcccacagccggtacggccctcaggccgtggacgcccgg

SEQ ID NO: 121 - DNA, 834 bp, synthetic construct
  misc_feature 1..834: note = Synthetic polynucleotide
  source 1..834: mol_type = other DNA
atgtgccggcggcccgactgcggcttcagcttcagccccggccccgtgatcctcctttggtgctgcttgctcttgcccatcgtgagcagcgccgccgtctccgtcgctcctaccgccgccgagaaggtccccgccgagtgccccgagctcacccgccgctgcctcctcggcgaggtcttcgagggcgacaagtacgagtcctggctcagacctctcgtcaacgtcaccggccgcgacggcccactctcccagctcatccgctaccgccccgtcacaccggaggccgccaactccgtcctcctcgacgaggccttcctcgacaccctcgccctcctctacaacaaccccgaccagctccgagccctgctgaccctgctgtcctccgacaccgctcctcgctggatgaccgtcatgcgcggctactccgagtgcggcgacggctccccagctgtgtacacctgcgtcgacgacctctgcagaggctacgacctgacgcgcctctcctacggccgctccatcttcaccgagcacgtcctcggcttcgagctcgtgcctccctccctcttcaacgtcgtcgtcgccatccgcaacgaggccacccgcaccaaccgcgccgtccgcctccccgtcagcacagccgctgcaccagagggcatcaccctcttctacggactgtacaacgccgtcaaggagttctgcctccgccaccagctcgacccacctctgctgaggcatctggacaagtattacgccggcctcccaccagagctgaagcagacccgcgtcaacctccccgcccactcccgctacggaccacaggccgtcgacgcccgc

SEQ ID NO: 122 - DNA, 834 bp, synthetic construct
  misc_feature 1..834: note = Synthetic polynucleotide
  source 1..834: mol_type = other DNA
atgtgccggcggcccgactgcggcttcagcttcagccccggccccgtgatcctcctctggtgctgccttttgctccccatcgtgagcagcgccgccgtctccgtcgctcctaccgccgccgagaaggtccccgccgagtgccccgagctcacccgccgctgcctcctcggcgaggtcttcgagggcgacaagtacgagtcctggctcagacctctcgtcaacgtcaccggccgcgacggcccactctcccagctcatccgctaccgccccgtcaccccagaggccgccaactccgtcctcctcgacgaggccttcctcgacaccctcgccctcctctacaacaaccccgaccagctcagggcccttctaaccctgctgtcctccgacaccgcccctcgctggatgaccgtcatgcgcggctactccgagtgcggcgacggctccccggccgtgtacacctgcgtcgacgacctctgcagaggatacgacctcacccggctctcctacggccgctccatcttcaccgagcacgtcctcggcttcgagctcgtcccaccctccctcttcaacgtcgtcgtcgccatccgcaacgaggccacccgcaccaaccgcgccgtccgcctccccgtcagcacagccgcagccccagagggcatcaccctcttctacggcctgtataacgccgtcaaggagttctgcctccgccaccagctcgacccgcctctgctgaggcacctggacaagtattacgccggcctccctcctgagctgaagcagacccgcgtcaacctccccgcccactcccgctatggaccacaggccgtcgacgcccgc

SEQ ID NO: 123 - DNA, 2094 bp, synthetic construct
  misc_feature 1..2094: note = Synthetic polynucleotide
  source 1..2094: mol_type = other DNA
atggagagcagaatttggtgcttggtggtgtgtgttaacctctgcattgtttgcctcggcgcagcagtgtctagcagctctactcggggaacatccgcaacccacagccaccatagctcacacaccaccagcgccgcccactccagatccggtagcgtgagccagcgggtgactagcagccagactgtgtcccatggcgtcaacgagacaatctacaacaccaccctgaagtacggcgacgtcgtgggcgtgaacacaacgaagtacccttacagggtgtgtagcatggctcagggcaccgacttgatccggttcgagagaaatattgtatgcaccagcatgaagccaattaacgaggatctggacgaaggcatcatggtagtgtataagagaaacatagttgcacacactttcaaggtgagggtctaccagaaggtgctgaccttccgccgaagctatgcttacatccatactacctacctgctcggttctaacaccgagtatgtggcacctccaatgtgggagatccaccacatcaattctcatagccagtgttacagctcttacagccgggtgatagccggcaccgtcttcgtggcctaccatagagattcatacgagaacaagaccatgcagctgatgccagacgactacagcaacacccattccaccaggtatgtgacagtcaaggaccaatggcactcacgcggctccacctggctgtacagggagacttgcaacctcaattgcatggtgaccatcaccaccgcccgcagcaagtacccgtaccacttcttcgccaccagcaccggagatgtcgtggacatcagccctttctataacggcactaacagaaacgccagttacttcggagagaatgccgacaagttcttcatcttcccgaactacactattgtgagcgatttcggtcgccctaactccgccctggagacacaccgcctggttgccttcctggagagagccgattccgtgatcagctgggacatccaggacgagaagaatgtgacctgtcagctcactttctgggaggcctccgagaggactatccggagcgaggccgaggactcataccatttcagcagcgccaagatgaccgccaccttcctgtcaaagaagcaggaggtgaacatgtcagatagcgctctggactgtgtgcgcgacgaggctattaacaagctgcagcagatcttcaatacctcctacaatcagacctacgagaagtatggtaacgtgtcagtattcgagacaactggcggcctcgtggtgttctggcagggaatcaagcagaagtccctggtggagcttgaaagactcgccaaccggagcagcctgaacctgacccacaacaggacaaagagatctacagatggtaacaacgccacccatctgagcaacatggagtccgtgcacaacctggtgtacgcccagctccagttcacatacgacaccctgagaggctacattaatagagccctcgcccaaatcgcagaggcctggtgcgtggaccagaggcgaaccctggaggtgttcaaggaattgagcaagatcaatccaagcgccatcttgagcgcaatctataacaagccgattgcggccagattcatgggcgacgtgttgggcctggcctcctgcgtcactatcaaccagacctctgtcaaggtgctcagagatatgaacgttaaggagtccccaggcagatgctatagcagacctgtcgtgattttcaatttcgccaactcaagctacgtgcagtacggccagctcggcgaagataacgagatcctgctgggcaaccacagaaccgaagagtgccagctgccttccctgaagattttcatcgctggcaactccgcttacgagtacgtggattacctgttcaagagaatgatcgacctcagcagcatcagcaccgtggacagcatgatcgccttagacattgaccctctggagaacacagatttcagggtgctggaactatactctcagaaggagctccggtctagcaacgtgttcgatctggaggagatcatgcgggagttcaattcctacaagcagcaccaccaccatcatcac

SEQ ID NO: 124 - DNA, 2094 bp, synthetic construct
  misc_feature 1..2094: note = Synthetic polynucleotide
  source 1..2094: mol_type = other DNA
atggagtcaagaatctggtgtcttgtggtgtgcgtgaacttgtgtatcgtgtgtttgggcgcagccgtgtcctcaagcagcactagaggcactagcgccacccactcccatcatagctcacacaccacaagcgccgcccactctaggtcaggcagcgtgtctcagcgcgtgacctctagccagactgtgagtcacggagtcaatgaaaccatctacaacacaacactgaagtacggagacgtcgtgggcgtgaatacaaccaagtacccatacagggtgtgcagcatggctcagggcactgacctcatcagattcgagcggaacattgtgtgcacctcaatgaagcctatcaacgaggatttggatgaaggcatcatggtcgtgtacaagagaaacatcgtcgctcataccttcaaggtgagagtgtatcagaaggtgctgaccttcagacgtagctacgcttacattcacaccacctacctgctgggcagcaacaccgagtatgtggctcctcctatgtgggagatacatcacatcaacagccattctcagtgctatagttcttatagcagggtcatcgccggcaccgtcttcgtggcctaccatagagatagctacgagaacaagaccatgcagttgatgccggacgattacagcaatacccatagcactaggtacgtgactgtgaaggaccagtggcactcccggggtagcacctggctttacagggaaacctgcaatctgaactgcatggtgactattaccaccgccaggagcaagtatccttaccacttcttcgctacatctactggagacgtggtcgatatctctcctttctacaatggcacaaacagaaatgcttcatatttcggcgagaatgccgacaagttcttcatcttcccaaactacaccattgtgtccgacttcggaagacctaattccgccctggaaacccatagactggtcgcattcctggaaagggccgactccgtcatttcatgggacatccaggatgagaagaacgtcacctgtcagctcacattctgggaagcgagcgaaagaacaattcgcagcgaagccgaggacagctatcatttcagcagtgctaagatgaccgccactttcctgtctaagaagcaggaggtgaacatgtccgacagcgccttggattgcgtgagagacgaagctattaacaagctgcagcagatcttcaacacctcctacaaccagacttacgagaagtatggcaatgtgagtgtgttcgagacaaccggcggcctggtagtattctggcagggcatcaagcagaagtcactggtggagcttgagaggctggccaatagatccagcctgaacctgacccacaaccggacaaagagatctaccgacggaaacaacgccactcatctttccaatatggagagcgtgcacaacctggtgtacgcgcagctccagttcacctacgacacactgaggggctacataaacagggccctggcacagatcgccgaagcctggtgcgtggaccagagaaggaccctggaggttttcaaggagctgagcaagattaatccgtccgctatcctgagcgcaatatacaataagccaatcgccgccagattcatgggcgacgtgttgggactggccagttgcgtcacaataaaccagacctctgtaaaggtcctgagggacatgaatgtcaaggagagcccgggcaggtgctacagccgtcctgtggtgattttcaacttcgctaattcatcttacgtccagtacggccagctgggcgaagacaatgagatcttactgggcaaccataggactgaggagtgccagctgccgagtctgaagattttcatagccggcaatagcgcatatgaatatgtagactacctgttcaagaggatgattgacctctctagcatctcgaccgtggacagcatgatcgccctcgacatcgaccctctggagaacacagacttccgggtcctcgaactgtacagccagaaggagcttaggagctccaacgtgttcgatcttgaggagatcatgagggagttcaatagctataagcaacatcaccaccatcatcac

SEQ ID NO: 125 - DNA, 2094 bp, synthetic construct
  misc_feature 1..2094: note = Synthetic polynucleotide
  source 1..2094: mol_type = other DNA
atggagagcagaatttggtgtctcgtggtgtgtgttaatctctgcatcgtgtgccttggcgccgccgtgtccagcagctccaccaggggcaccagcgcaacacacagccaccactctagccacacaaccagcgccgcccactctcgttcaggctccgtttcacagagggtgacctcctctcaaaccgtgtctcatggagtgaatgaaaccatttataacacaacactgaagtatggcgacgtggtgggcgtcaacaccaccaagtatccttaccgggtttgttcaatggcccagggcaccgatctcatcagattcgagcgaaatatcgtgtgtacatccatgaagcctatcaacgaagacctggacgagggaattatggtcgtgtacaagagaaatattgtggcccacactttcaaggtgagagtgtaccagaaggtgttgacattcaggcggtcctacgcctacatccacaccacttatctgttgggatccaacacagagtacgtcgcaccgcctatgtgggaaatacatcacatcaattcccattctcagtgctattctagctactccagagtgatcgccggcaccgtgttcgtggcctaccaccgcgatagctacgagaataagaccatgcagctgatgcctgacgattacagcaacactcattccacacgctacgtgaccgtgaaggatcagtggcacagccgcggcagcacctggctgtaccgggaaacctgcaacctgaactgcatggtgacaataacaaccgcacgtagcaagtacccataccacttcttcgccacctccaccggtgacgtggtcgacatcagccctttctacaatggcaccaacagaaatgcctcctacttcggcgagaacgccgacaagttcttcatcttccctaattatacaattgtgagcgacttcggcaggcctaacagcgccctggagactcatcgcttggtggctttcctggaacgcgctgacagcgtcatctcttgggatatccaagatgagaagaacgtgacctgccagctgaccttctgggaggccagcgagaggacaatcagaagcgaagccgaggactcttaccatttcagttcagctaagatgaccgccaccttcctgagcaagaagcaggaagtgaatatgtccgattccgctcttgactgcgtcagggacgaagccatcaacaagctccagcagattttcaatacttcttataatcagacctatgagaagtacggcaatgtcagcgtcttcgagacgaccggcggcctggttgtgttctggcaaggaatcaagcagaagtcactggtggagcttgagcggctggccaacagatccagcttgaacctgacccataatcgcaccaagcggagtaccgatggcaacaacgccacacacctcagcaatatggaaagcgtgcacaaccttgtgtacgctcagctgcagttcacctacgatacccttagaggctacatcaacagagccctggcccagatcgcagaggcatggtgcgtggaccagcggcggaccctggaggtgttcaaggagctctccaagatcaacccatccgccattctctccgccatctacaacaagcctattgccgctcggttcatgggcgatgtgctgggactggccagctgcgtgaccatcaaccaaacctcagtgaaggtgctcagagacatgaacgtcaaggagtctccaggcagatgttacagcagacctgtggtgatcttcaacttcgccaattcttcctacgtgcagtacggccaactgggtgaagacaacgagattctgttaggcaaccacaggactgaggagtgtcagctgccgagcctgaagatcttcatcgctggaaacagcgcatacgagtacgtggactacctcttcaagaggatgatcgacttgtcatctatctccacggttgattccatgatcgccttggacatcgatcctctggagaataccgacttcagagtgctggagctctacagccagaaggagcttaggtctagcaatgtgttcgacctggaggagatcatgagggagttcaatagctataagcagcatcaccaccaccatcac

SEQ ID NO: 126 - DNA, 2094 bp, synthetic construct
  misc_feature 1..2094: note = Synthetic polynucleotide
  source 1..2094: mol_type = other DNA
atggagagcagaatctggtgcctcgtggtgtgcgtgaacctctgtatcgtctgcttaggagccgccgtgagcagtagctctaccagaggcacatccgccacccacagccaccactcttcacacaccaccagcgccgcccactccagatcaggcagcgtatcccagagagtgaccagcagccagaccgtgtcacatggagtgaatgaaacaatttacaacaccaccctcaagtacggcgacgtagtgggagtgaacactactaagtacccataccgcgtgtgtagcatggcccagggcaccgatctgatccgattcgagagaaacatcgtttgcaccagcatgaagcctatcaacgaggacctggatgagggcatcatggtggtgtacaagaggaacatcgtggcccacacgttcaaggttagggtgtaccagaaggtgctgactttccgaagaagctatgcctacattcacactacatacctgctcggcagtaacaccgagtacgtggcgccaccgatgtgggaaatacaccatattaattctcatagtcagtgctattccagctacagcagggtgatcgccggaaccgttttcgtggcttatcatagagattcctacgagaacaagaccatgcagctgatgccagacgactatagcaacacgcatagcacccgctacgtgaccgtgaaggaccagtggcattcaagaggatccacctggctctacagagagacatgcaatctgaactgcatggtgaccatcaccaccgcccggtccaagtacccttatcacttcttcgccacaagcaccggcgatgtggtggacatttccccattctacaacggaaccaaccggaacgcctcttacttcggcgagaacgccgacaagttcttcatcttcccaaattataccatcgtgagcgacttcggaagacctaacagcgccctggagacacacagactggtggccttcctcgagcgcgccgactccgtgatctcctgggacatccaggacgagaagaacgtgacttgtcagctgacattctgggaggccagcgaacggaccatcagaagcgaggctgaagactcctaccacttcagctccgccaagatgaccgccactttcctgtcaaagaagcaggaggtgaacatgagcgacagcgccttggattgcgtgagagatgaggccatcaacaagcttcaacagatcttcaacacatcctacaaccagacgtacgagaagtacggaaacgtgagcgtgttcgaaaccaccggcggcttagtggtgttctggcagggaatcaagcagaagtctctggtggagctggagagactggctaacagatcctctctaaacctgacacacaacagaaccaagcggagcacagacggcaataatgccacacacctgagcaacatggaaagcgtccacaacctcgtctatgcccaactgcagttcacctacgacaccctccgaggctacatcaacagagccctggcccagatcgccgaggcttggtgtgtggatcagagacggaccctggaggtgttcaaggagttgagcaagatcaacccgtccgccatcttgagcgctatatacaacaagccaattgctgcgcggttcatgggcgacgtgctgggcctcgcctcatgtgtgaccattaatcaaacaagcgtcaaggtcctgagggatatgaacgttaaggagagcccaggcaggtgctatagcagacctgtggtgattttcaacttcgccaacagcagctacgtgcagtacggccagctgggcgaggacaacgagatcctgctgggcaaccaccgcactgaggagtgccagctgccaagtctgaagatattcatcgcgggaaattcagcttacgagtatgtagactacctgttcaagagaatgatagatcttagcagcatctccactgtggacagtatgatagctcttgatattgacccactggagaataccgacttcagagtgttggagctgtacagtcagaaggagctcaggagctccaatgtgttcgacctggaggagatcatgagggaattcaatagctacaagcagcaccaccaccatcatcac

SEQ ID NO: 127 - DNA, 2094 bp, synthetic construct
  misc_feature 1..2094: note = Synthetic polynucleotide
  source 1..2094: mol_type = other DNA
atggaatcccgaatctggtgcctcgttgtgtgcgtgaatttgtgcatcgtgtgtttgggcgccgccgtctcttcttcctccacacgtggtaccagcgcaacacactcccaccactcaagccacaccacgtccgcggcacacagcagaagcggcagtgtgagccaaagggtgaccagcagccagaccgtgagtcacggcgtgaacgagacaatctacaataccacactcaagtacggcgatgtggtgggcgtcaacaccaccaagtatccttacagagtctgttccatggcccagggcactgacctgatccggttcgaaagaaacatagtgtgcacctccatgaagcctatcaatgaggacctcgatgaaggcattatggtggtgtacaagaggaatattgtggcccataccttcaaggtgagagtgtaccagaaggtgctgaccttcagacggagctacgcctacatccatacaacctacctgctgggaagcaacaccgagtacgtggctcctccaatgtgggagatccaccacatcaatagccacagccagtgctactccagctacagcagagtgattgctggcaccgtcttcgtggcttaccacagagacagctatgagaacaagacaatgcagctcatgccagacgactactctaacacacattcaacccggtatgtgaccgtgaaggaccagtggcactcaagaggcagcacatggctctaccgagagacatgtaacctgaactgcatggttacaatcaccactgcaaggtctaagtacccatatcacttcttcgccacctctaccggagacgtggtggacatcagcccattctacaatggcaccaatcggaacgcaagctacttcggagagaacgccgacaagttcttcatcttcccgaactacaccatcgtgtccgatttcggcaggccaaacagcgccctggagacacaccggctggtggccttcctggagcgcgctgactccgttatctcttgggacatccaggatgagaagaatgtgacctgccaactgacattctgggaggcatccgagcggactatcagaagcgaggccgaggacagctaccacttcagcagcgctaagatgactgctaccttcctgtccaagaagcaggaggtgaacatgtctgattccgctctggactgcgtgagggacgaggctatcaacaagctccagcagatattcaatacttcctacaaccagacctacgagaagtacggtaacgtcagcgttttcgaaaccaccggcggcctggtcgtgttctggcagggaatcaagcagaagtcccttgtcgagctcgagagactggccaaccggtctagcctcaatctgacacacaataggaccaagagatctactgacggcaataacgccacacacctctccaacatggagagtgttcataacctggtttacgcccagctgcagttcacttacgataccctccgcggctacatcaacagggccctggcgcagatcgccgaggcctggtgcgtggatcaaagaaggaccctggaggtcttcaaggaactcagcaagatcaacccatctgctatcctgagcgccatctacaacaagccaatcgccgcccggttcatgggcgacgtcctgggcttggctagctgcgtgaccatcaatcagaccagcgtcaaggtgcttcgcgacatgaacgtcaaggagtcacctggccgctgttactcaaggccagtcgtgatcttcaatttcgccaatagctcctacgtgcagtacggacagttgggcgaggacaatgaaatactcctgggcaaccaccgcaccgaggagtgtcagctgccaagcctgaagatcttcatcgcgggaaactccgcttacgagtatgtggactacctgttcaagagaatgattgatctgagcagcatctccaccgtggacagcatgattgctctggatattgatcctctggagaacaccgatttccgcgtgctggagctgtacagccagaaggaattaaggagcagtaatgtgttcgacctggaggagatcatgagggagttcaacagttacaagcagcaccaccatcaccaccac

SEQ ID NO: 128 - DNA, 2094 bp, synthetic construct
  misc_feature 1..2094: note = Synthetic polynucleotide
  source 1..2094: mol_type = other DNA
atggagtccagaatctggtgcttggtggtgtgcgtgaatctttgcattgtgtgcctcggcgccgccgtgagcagcagcagtactagaggtacctccgctacccacagccaccactcttcccatacaaccagcgccgcccactcacgtagcggctctgtgagccagagggtgacaagctcacagaccgtgagccacggcgtgaacgagactatctacaacactaccctgaagtacggcgatgtggtgggagtgaataccacaaagtacccgtacagggtgtgttccatggcccagggcaccgacctgattcgcttcgaaagaaacatcgtctgcaccagcatgaagcctatcaacgaggatttggatgagggtattatggtggtctacaagagaaatattgtggcccacaccttcaaggtcagagtgtaccagaaggtcctgacgttcaggagatcttacgcttacatccacaccacctaccttctgggctccaacaccgagtatgtggccccgcctatgtgggagatccaccacattaattcccactctcaatgctacagctcctattccagagtgatcgccggcacagtcttcgtggcctaccaccgggacagctatgagaacaagactatgcagctcatgccagacgactatagcaatactcatagcactagatatgtgactgtgaaggaccagtggcatagcagaggcagcacttggctgtaccgggaaacatgcaatcttaattgcatggtcaccataaccaccgcgagatccaagtacccttaccacttcttcgccacctccactggtgacgtcgtggacatctcccctttctataacggaacaaatagaaacgccagctacttcggtgagaacgccgacaagttcttcatcttccctaactacaccatagtgagcgatttcggcagaccgaactccgctctggagacacaccggctggtggccttcctggaacgggccgatagtgttatctcttgggatattcaagacgagaagaacgtcacctgtcagctgactttctgggaagccagcgagaggaccatcagaagtgaagctgaggatagctaccatttctctagtgccaagatgactgccaccttcctgtccaagaagcaggaggtcaacatgtccgacagcgccctcgactgtgtgagagacgaggctattaacaagctgcagcagattttcaacactagctacaatcagacatacgagaagtatggaaacgtgagcgtgttcgaaactaccggtggcctggtggtattctggcagggcatcaagcagaagtccctggtggaattggagagactggctaacaggtcgtccctgaacctgactcacaatagaacgaagaggagcacagacggcaataatgccacccatctgtccaatatggagagtgtgcacaatttggtgtatgcccagctgcagttcacctacgacaccctcaggggctacatcaacagagccctcgcccagatcgctgaagcctggtgcgtggatcagaggaggaccctggaggtcttcaaggaactgagcaagataaacccatccgccatcctcagtgccatttataacaagcctattgccgccaggttcatgggcgacgtgctgggcctggcttcctgtgtcacgattaatcagacctccgtgaaggtgctgagggacatgaacgtgaaggaaagccctggacggtgttacagccgaccagtagtgatcttcaacttcgccaactcctcatacgtgcagtatggccagctgggcgaggacaatgaaattctgctgggcaaccacaggaccgaagagtgccagctgcctagcctgaagatattcatcgccggtaatagcgcctacgagtacgtcgactatcttttcaagagaatgatcgatctgtctagcatttctaccgtggattccatgatcgctcttgacattgacccactggagaacacagacttcagggtgctcgagctgtattcccagaaggaactcaggtctagcaacgttttcgacctcgaggaaattatgagagagttcaactcgtacaagcaacaccatcaccaccatcac

SEQ ID NO: 129 - DNA, 2094 bp, synthetic construct
  misc_feature 1..2094: note = Synthetic polynucleotide
  source 1..2094: mol_type = other DNA
atggagagccggatctggtgcctcgtggtgtgcgtgaacttatgcatcgtgtgcctcggcgccgccgtgagctctagctctacccggggcaccagcgccacccacagccaccacagcagccacaccacctcggccgctcacagccggagcggcagcgtgagccagcgggtgacctccagccagaccgtgtcccacggcgtgaacgagacgatctacaacaccaccctgaagtacggcgacgtggtgggagtgaacacgaccaagtacccctaccgggtgtgcagcatggcccagggcaccgacctgatccggttcgagcggaacattgtgtgcaccagcatgaagcccatcaacgaggacctggacgagggcatcatggtagtgtacaagagaaacatcgtggcccacaccttcaaggtgcgggtgtaccagaaggtgctgaccttccggcggagctacgcctacattcacacaacatacctgctgggcagcaacaccgagtacgtggctcctcccatgtgggagatccaccacatcaactctcatagccagtgctacagcagctacagccgggtgatcgccggcaccgtgttcgtggcctaccaccgggacagctacgagaacaagaccatgcagctgatgcccgacgactatagcaacacacactccactcggtacgtgaccgtgaaggaccagtggcacagcagaggcagcacctggctgtaccgggagacttgcaacctgaactgcatggtgaccatcaccaccgcccggtctaagtacccttaccacttcttcgccaccagcaccggcgatgtggtggacatcagccccttctacaacggcaccaaccggaacgccagctacttcggcgagaacgccgacaagttcttcatcttccccaactacaccatcgtgagcgacttcggccggcccaacagcgccctggaaactcaccggctggtggccttcctggagcgggccgacagcgtgatcagctgggacatccaggacgagaagaacgtgacctgccagctgacattctgggaggccagcgagcggaccatccggagcgaggccgaggatagctatcacttcagcagcgccaagatgaccgccaccttcctgagcaagaagcaggaggtgaacatgagcgattctgcactggactgcgtgcgggacgaggccatcaacaagctgcagcagatcttcaacaccagctacaaccagacctacgagaagtacggaaacgtgagcgtgttcgagactaccggcggccttgtcgtgttctggcagggaatcaagcagaagtccctggtcgagctcgagcgactggccaacagaagcagcctgaacctgacccacaaccggaccaagcggagcaccgacggcaacaacgccacacacctgtctaacatggagtctgtgcacaacctggtgtacgcccagctgcagttcacctacgacaccctgcggggctacatcaaccgggccctggcccagatcgccgaggcatggtgcgtggaccagcggcggaccctggaggtgttcaaggagctctctaagatcaacccgtctgccatcctgagcgccatttacaacaagcctatcgccgcaagattcatgggcgacgtcctgggcctggccagctgcgtgacgatcaatcagaccagcgtgaaggtcctgcgggacatgaacgtcaaggagagccccggcaggtgctatagccggcccgtggtgattttcaacttcgccaatagctcttacgtgcagtacggtcagttaggcgaggacaacgagatcttactgggcaaccaccggaccgaggagtgccaactcccgagcctcaagattttcattgccggcaatagcgcatacgaatatgtggactacctgttcaagcggatgatcgacctgagcagcatcagcaccgtggacagcatgattgctctggacatcgaccctctggagaacaccgacttccgggtgctggagctgtacagccagaaggagctgcggagctctaatgtgttcgacctggaggagatcatgcgggagttcaactcatataagcagcaccaccaccatcatcac

SEQ ID NO: 130 - DNA, 2094 bp, synthetic construct
  misc_feature 1..2094: note = Synthetic polynucleotide
  source 1..2094: mol_type = other DNA
atggagagccggatctggtgcctcgtggtgtgcgtgaacctttgcatcgtgtgcttgggcgccgccgtgagcagcagctccacccggggcacctccgccacccactcccaccactcctcccacaccacctcagccgcccactctcgctccggctccgtctcccagcgcgtcacctccagccagaccgttagccacggcgtcaacgaaaccatctacaacaccaccctcaagtacggcgacgtcgtcggcgtaaacacaaccaagtacccctaccgcgtctgctccatggcccagggcaccgacctcatccgcttcgagcgcaacatcgtctgcacctccatgaagcccatcaacgaggacctcgacgagggcatcatggtcgtctacaagcgcaatattgtggcccacaccttcaaggtccgcgtctaccagaaggtcctcaccttccgccgctcctacgcctacatccacacaacctacctcctcggctccaacaccgagtacgtcgcccctcccatgtgggagatccaccacatcaacagccacagccagtgctactcctcctactcccgcgtcatcgccggcaccgtcttcgtcgcctaccaccgcgactcctacgagaacaagaccatgcagctcatgcccgacgactacagcaatacccacagcacccgctacgtcaccgtcaaggaccagtggcacagcagaggctccacctggctctaccgcgagacatgcaacctcaactgcatggtcaccatcaccaccgcccgctccaagtatccttaccacttcttcgccacctccaccggcgatgtcgtggacatctcaccattctacaacggcaccaaccgcaacgccagttacttcggcgagaacgccgacaagttcttcatcttccccaactacaccatcgtctccgacttcggccgccccaactccgccctcgagacacacagactggtggccttcctcgagcgcgccgactccgtcatctcctgggacatccaggacgagaagaacgtcacctgccagctcacattctgggaggcctccgagcgcaccatccgctccgaggccgaggactcataccatttctccagcgccaagatgaccgccaccttcctctccaagaagcaggaggtcaacatgagcgacagcgctctcgactgcgtccgcgacgaggccatcaacaagctccagcagatcttcaacacctcctacaaccagacgtacgagaagtatggaaacgtcagtgtcttcgaaaccacgggcggcctggttgtattctggcagggaataaagcagaagtccctcgtcgagcttgagcgcctcgccaaccgctcctccctcaacctcacccacaaccgcaccaagcgctccaccgacggcaacaacgctacccacctgtccaacatggagtccgtccacaacctcgtctacgcccagctccagttcacctacgacaccctccgcggctacatcaaccgcgccctcgcccagatcgccgaggcctggtgcgtcgaccagcgccgcaccctcgaggtcttcaaggagctgagtaagatcaaccctagcgcgatcctcagcgctatctataacaagccaatcgctgctaggttcatgggagacgtgctcggcctcgcctcctgcgtgaccatcaatcagacatccgtgaaggtgctgcgcgacatgaatgtcaaggagagcccaggccgctgttattcccggcccgtcgtcattttcaatttcgccaatagctcttacgtccagtacggccagctcggcgaggacaacgagatcctgctgggcaaccaccgcaccgaggagtgccagctgcctagcctcaagattttcattgccggcaattccgcttacgaatacgtggactacctcttcaagcgcatgatcgacctctcctccatctccaccgtcgactccatgatcgccctggatatcgaccctctcgagaacaccgacttccgcgtgttggagctctactcccagaaggagctcagatccagcaacgtattcgacctcgaggagatcatgcgcgagttcaactcctataagcagcaccaccaccatcatcac

SEQ ID NO: 131 - DNA, 2094 bp, synthetic construct
  misc_feature 1..2094: note = Synthetic polynucleotide
  source 1..2094: mol_type = other DNA
atggagagccggatctggtgcttggtggtgtgcgtgaacttgtgcatcgtgtgcttgggcgccgccgtgagcagctctagcacccggggcaccagcgccacccacagccaccacagcagccacacgacctccgccgcccactcacggagcggcagcgtgagccagcgggtgaccagctcacagaccgtgtcccacggcgtgaacgagacgatctacaacaccaccctgaagtacggcgacgtggtgggcgtcaacactaccaagtacccctaccgggtgtgcagcatggcccagggcaccgacctgatccggttcgagcggaatattgtgtgtaccagcatgaagcccatcaacgaggacctggacgagggcatcatggtggtctacaagagaaacattgtggcccacaccttcaaggtgcgggtgtaccagaaggtgctgaccttccggcggagctacgcctacatccatacaacctacctgctgggcagcaacaccgagtacgtggcgcctcccatgtgggagatccaccacatcaactctcactcgcagtgctacagcagctacagccgggtgatcgccggcaccgtgttcgtggcctaccaccgggacagctacgagaacaagaccatgcagctgatgcccgacgactattctaacacccactccaccagatacgtgaccgtgaaggaccagtggcacagcaggggcagcacctggctgtaccgggagacttgcaacctgaactgcatggtgaccatcaccaccgcccggagtaagtatccatatcacttcttcgccaccagcacgggcgacgttgtggacatcagccccttctacaacggcaccaaccggaacgccagctacttcggcgagaacgccgacaagttcttcatcttccccaactacaccatcgtgagcgacttcggccggcccaacagcgccctggaaacccaccggctggtggccttcctggagcgggccgacagcgtgatcagctgggacatccaggacgagaagaacgtgacctgccagctgactttctgggaggccagcgagcggaccatccggagcgaggccgaagactcctaccacttcagcagcgccaagatgaccgccaccttcctgagcaagaagcaggaggtgaacatgagcgattcagctctggactgcgtgcgggacgaggccatcaacaagctgcagcagatcttcaacaccagctacaaccagacttacgagaagtatggaaacgtgagcgtgttcgagacaaccggcggcctcgtggtgttctggcagggtatcaagcagaagtctctcgtggagctggagagactggccaacagaagcagcctgaacctgacccacaaccggaccaagcggagcaccgacggcaacaacgctacccatctgtctaacatggagtcagtgcacaacctggtgtacgcccagctgcagttcacctacgacaccctgcggggctacatcaaccgggccctggcccagatcgccgaggcctggtgcgtggaccagcggcggaccctggaggtgttcaaggagctgtccaagatcaacccttccgccatcctgagcgccatttataataagccgatcgccgcccggttcatgggcgatgttctgggcctggccagctgcgtcaccattaatcagaccagcgttaaggtcctgcgggacatgaatgtcaaggagagccccggcaggtgctactcccgccccgtggtgatattcaacttcgccaactctagctacgtgcagtacggccaactaggcgaggacaacgagatcttgctcggtaaccaccggaccgaggagtgccagttaccttccctgaagattttcatcgcgggcaactccgcctacgagtatgtggactacctgttcaagcggatgatcgatctttctagcatcagcaccgtggacagcatgatagccctggacatcgacccactggagaacaccgacttccgggtgctggagctgtacagccagaaggagcttcggagcagcaatgtgttcgacctggaggagatcatgcgggagttcaattcttacaagcagcaccaccaccatcatcac

SEQ ID NO: 132 - DNA, 2094 bp, synthetic construct
  misc_feature 1..2094: note = Synthetic polynucleotide
  source 1..2094: mol_type = other DNA
atggagagccggatctggtgcctcgtggtgtgcgtgaacctctgcatcgtgtgcctcggcgccgccgtgtcttcatcctccacccggggcacctccgccacccactcccaccactcctcccacaccactagtgccgcccactcacgctccggctccgtctcccagcgcgtcacctcatcccagacagtgagccacggcgtcaacgaaaccatctacaacaccaccctcaagtacggcgacgtcgtgggcgtgaacactacaaagtacccctaccgcgtctgctccatggcccagggcaccgacctgatccgcttcgagcgcaacatcgtctgcacctccatgaagcccatcaacgaggacctcgacgagggcatcatggtcgtctacaagaggaacattgtggcccacaccttcaaggtccgcgtctaccagaaggtcctcaccttccgccgctcctacgcctacatccacactacgtacctcctcggctccaacaccgagtacgtcgcccctcccatgtgggagatccaccacatcaactcgcacagccagtgctactcctcctactcccgcgtcatcgccggcaccgtcttcgtcgcctaccaccgcgactcctacgagaacaagaccatgcagctcatgcccgacgactatagcaacacacatagcacccgctacgtcaccgtcaaggaccagtggcatagcagaggctccacctggctctaccgcgagacgtgcaacctcaactgcatggtcaccatcaccaccgcccgcagcaagtatccatatcacttcttcgccacctccacaggagacgtggtcgacatctcgcctttctacaacggcaccaaccgcaacgctagctacttcggcgagaacgccgacaagttcttcatcttccccaactacaccatcgtctccgacttcggccgccccaactccgccctcgagactcaccgcttggtggccttcctcgagcgcgccgactccgtcatctcctgggacatccaggacgagaagaacgtcacctgccagctgaccttctgggaggcctccgagcgcaccatccgctccgaggccgaggacagctaccacttcagcagcgccaagatgaccgccaccttcctctccaagaagcaggaggtcaacatgagcgacagcgcccttgactgcgtccgcgacgaggccatcaacaagctccagcagatcttcaacacctcctacaaccagacttatgagaagtacggaaacgtctccgttttcgagacaacaggaggcctggttgtcttctggcagggcattaagcagaagtccctcgtcgagctggagagactcgccaaccgctcctccctcaacctcacccacaaccgcaccaagcgctccaccgacggcaacaatgctacacacctgagcaacatggagtccgtccacaacctcgtctacgcccagctccagttcacctacgacaccctccgcggctacatcaaccgcgccctcgcccagatcgccgaggcgtggtgcgtcgaccagcgccgcaccctcgaggtcttcaaggagctgtccaagatcaaccctagcgccatcctgtccgcaatctataacaagcctatcgcggctaggttcatgggcgatgtgctcggcctcgcctcctgcgtgactattaatcagaccagcgtcaaggtgctgcgcgacatgaacgtgaaggagagccctggccgctgctattccaggcccgtcgtcatcttcaatttcgccaattccagctatgtccagtacggccagctcggcgaggacaacgagatcctgcttggcaaccaccgcaccgaggagtgtcagctccctagcctgaagattttcattgccggcaatagcgcttatgagtatgtggactacctcttcaagcgcatgatcgacctctcctccatctccaccgtcgactccatgatcgccctggacatcgacccactggagaacaccgacttccgcgtgctcgaactctactcccagaaggaactgagatcaagcaacgtgttcgacctcgaggagatcatgcgcgagttcaactcttataagcagcaccaccaccatcatcac

SEQ ID NO: 133 - DNA, 642 bp, synthetic construct
  misc_feature 1..642: note = Synthetic polynucleotide
  source 1..642: mol_type = other DNA
atgctcagacttctcctcagacaccacttccactgcctcttgctttgtgccgtctgggccacaccttgcctcgccagcccttggagcaccttgacagccaaccagaacccttcccctccttggtcaaagttgacctacagcaagcctcacgacgctgctaccttctactgtccattcctgtaccctagccctccaagatctccgctgcagttcagcggcttccagagggtgtctaccggacctgagtgcaggaatgagacgctgtacctgctgtacaacagagagggccagaccctggtggaaagaagctccacctgggtcaagaaggtaatctggtacctgagcggcagaaaccagacaatactccagagaatgccacggaccgctagcaagcctagcgatggaaacgtgcagattagcgtggaggacgcaaagattttcggcgcccacatggtgccaaagcagacaaagctgctgcggttcgtggtcaacgacggcacccggtaccagatgtgcgtgatgaagctggagagctgggctcacgtgttcagagattactctgtgagcttccaagtgcggctcaccttcaccgaagccaacaatcagacctacactttctgtactcaccctaacctgatcgtg

SEQ ID NO: 134 - DNA, 642 bp, synthetic construct
  misc_feature 1..642: note = Synthetic polynucleotide
  source 1..642: mol_type = other DNA
atgcttagactcctcctcagacaccacttccattgtctccttttgtgcgccgtgtgggccaccccttgccttgcatcaccttggtctaccctcaccgccaaccagaaccctagccctccttggagtaagttaacatactctaagccgcacgacgccgccaccttctactgtcctttcctctacccaagcccacctcgtagcccacttcagttctctggattccagagagtttcaacaggccctgagtgtcggaacgagactctgtacctgttgtataacagagagggacagaccctggtggagcggtcctccacctgggtgaagaaggtgatctggtatctgagcggcagaaaccagaccatcctgcagcggatgccaaggaccgctagcaagccaagcgacggcaatgtgcagattagcgtggaggatgctaagattttcggcgcacacatggttcctaagcagaccaagctgttacggttcgtggtgaacgatggaactcggtaccaaatgtgcgtgatgaagctggagtcatgggcacatgtgttccgtgactactctgtttctttccaggtgcgcctgaccttcaccgaggccaataaccagacatacaccttctgtacgcacccaaatctgatcgta

SEQ ID NO: 135 - DNA, 642 bp, synthetic construct
  misc_feature 1..642: note = Synthetic polynucleotide
  source 1..642: mol_type = other DNA
atgctcagacttttgctcagacaccacttccactgtttgttgttatgtgccgtgtgggctaccccttgcctcgcatctccgtggtccacactcacagccaaccagaatccttctcctccttggagcaagctcacatatagcaagcctcacgacgcggcaaccttctactgcccattcctgtatccttctcctccgcggagccctctgcagttctccggattccagagagtgtccaccggtcctgagtgcagaaatgaaacactgtatcttctctacaacagagagggccagacccttgtggagagaagcagcacctgggtgaagaaggtcatttggtatctgtctggcagaaaccagaccatactgcagcggatgccaagaacagcctccaagccatccgacggtaacgtgcagatctccgtggaggacgccaagattttcggcgcccacatggtgccaaagcagaccaagctgctgagattcgtggtgaacgatggcaccaggtaccagatgtgcgttatgaagcttgagtcctgggctcacgtgttcagagactactctgtgagcttccaggtgagactgacattcacagaggccaacaaccagacttacaccttctgcacgcatcctaatctgatcgtg

SEQ ID NO: 136 - DNA, 642 bp, synthetic construct
  misc_feature 1..642: note = Synthetic polynucleotide
  source 1..642: mol_type = other DNA
atgctcagacttcttctcagacaccacttccactgtttgcttctctgcgcagtgtgggcaaccccttgccttgcttccccttggtcgactctcaccgccaaccagaatccaagccctccttggagcaagctcacttacagcaagccgcacgacgccgccaccttctactgtcctttcctgtaccctagccctccaagatctcctctgcaattctctggattccagagagtgagcaccggcccagagtgccggaacgagactctgtatctgctgtacaatagggagggacaaaccctggtggagaggagcagcacatgggtgaagaaggtgatctggtacctgagcggcagaaaccagaccatcctgcagagaatgccacggaccgccagcaagccaagcgatggcaacgtccagattagcgtggaagacgccaagatcttcggagcccacatggtgcctaagcagaccaagcttctgcgattcgtggtgaacgacggtacccgctaccaaatgtgcgtgatgaagctggagtcatgggcccacgtcttccgcgactacagcgtatccttccaggtgaggcttaccttcaccgaggccaacaaccaaacctacacattctgcacccatccaaatttgattgtg

SEQ ID NO: 137 - DNA, 642 bp, synthetic construct
  misc_feature 1..642: note = Synthetic polynucleotide
  source 1..642: mol_type = other DNA
atgctcagactattgttgagacatcacttccattgcctccttttgtgcgccgtgtgggctaccccttgcctcgcctcaccttggagcaccttgaccgccaaccagaacccgagccctccgtggtcaaagctcacctacagcaagcctcacgacgccgcaaccttctattgtccattcctgtacccttctccgccgaggtcccctcttcagttcagcggattccagagagtgtctaccggaccagaatgcagaaacgaaacactgtatctgctgtacaaccgggagggccagaccctggtcgagcggagctctacctgggtcaagaaggttatatggtatctgagcggcaggaaccagaccatcctgcagcgcatgcctagaaccgctagcaagccaagcgacggcaacgttcagatctccgtggaggacgctaagatcttcggcgcccatatggtgccaaagcagactaagctgctgagattcgtggtaaacgacggcacaagatatcagatgtgcgtgatgaagctggagagctgggctcatgtgttcagggactactccgtgagtttccaggtgaggctgacattcaccgaggctaataatcagacctacaccttctgcactcacccaaatctgatcgtg

SEQ ID NO: 138 - DNA, 642 bp, synthetic construct
  misc_feature 1..642: note = Synthetic polynucleotide
  source 1..642: mol_type = other DNA
atgctcagattattgctcagacaccacttccactgcctcctcttgtgcgccgtgtgggcgacaccgtgtctcgcaagcccttggtccacactaacggccaaccagaaccctagccctccttggagcaagctcacttatagcaagccacacgatgcggccactttctactgtcctttcctgtatccatcccctcctagatctcctctgcagttcagcggattccagagagtatctactggccctgagtgcagaaatgaaaccctctatctcctgtacaatcgggagggccagactttggtggagcgcagctccacctgggtgaagaaggtgatctggtacctgagcggcagaaaccagaccatcctacagaggatgccaaggaccgccagcaagccatctgacggcaacgtgcagatctctgtggaggacgccaagatcttcggagcccatatggtgcctaagcagacaaagctgttgaggttcgtcgtgaatgacggcacaagataccagatgtgtgtgatgaagctggagagctgggctcacgtgttccgagactacagcgtctcgttccaggtgagactgacattcaccgaggcaaacaaccagacctacaccttctgtacgcaccctaacctgatcgtt

SEQ ID NO: 139 - DNA, 642 bp, synthetic construct
  misc_feature 1..642: note = Synthetic polynucleotide
  source 1..642: mol_type = other DNA
atgcttcggctcctccttcggcaccacttccactgcctcctcctctgcgccgtgtgggccacaccttgcctcgccagcccctggagcaccctcaccgccaaccagaaccccagccctccgtggtctaagttaacctacagcaagccccacgacgccgccaccttctactgccccttcctgtacccttcaccgccgcggagcccgctgcagttcagcggcttccagcgggtgagcaccggccccgagtgccggaacgagacgctgtacctgctgtacaaccgggagggccagaccctggtggagcggagcagcacctgggtgaagaaggtgatctggtacctgagcggccggaaccagaccatcctgcagcggatgccccggaccgcctcaaagccaagcgacggcaacgtgcagatcagcgtggaggacgccaagatcttcggcgcccacatggtgcccaagcagaccaagttgctgcgcttcgtggtgaacgacggcacccggtaccagatgtgcgtgatgaagctggagagctgggcccacgtgttccgggactacagcgtgagcttccaggtgcggctgacattcaccgaggccaacaatcagacctacaccttctgcacccaccccaacctgatcgtg

SEQ ID NO: 140 - DNA, 642 bp, synthetic construct
  misc_feature 1..642: note = Synthetic polynucleotide
  source 1..642: mol_type = other DNA
atgttacggctccttctccgccaccacttccactgcctcttactctgcgccgtgtgggccactccatgccttgccagcccctggagcaccttgaccgccaaccagaaccccagcccaccttggagtaagctcacctacagcaagccccacgacgccgccaccttctactgccccttcctgtatccgagcccaccgcggagcccgctgcagttcagcggcttccagcgggtgagcaccggccccgagtgccggaacgaaaccctgtacctgctgtacaaccgggagggccagaccctggtggagcggagcagcacctgggtgaagaaggtgatctggtacctgagcggccggaaccagaccatcctgcagcggatgccccggaccgctagtaagcctagcgacggcaacgtgcagatcagcgtggaggacgccaagatcttcggcgcccacatggtgcccaagcagaccaagctgcttaggttcgtggtgaacgacggcacccggtaccagatgtgcgtgatgaagctggagagctgggcccacgtgttccgggactacagcgtgagcttccaggtgcggctgaccttcaccgaggccaacaaccagacatacaccttctgcacccaccccaacctgatcgtg

SEQ ID NO: 141 - DNA, 642 bp, synthetic construct
  misc_feature 1..642: note = Synthetic polynucleotide
  source 1..642: mol_type = other DNA
atgctacggctcctactccgccaccacttccactgcttacttttgtgcgccgtgtgggccacaccatgcttggccagcccctggagcaccctcaccgccaaccagaacccctcacctccctggtccaagctcacctactccaagccccacgacgccgccaccttctactgccccttcctctatccatctcctccacgcagcccactccagttctccggcttccagcgcgtctccaccggccccgagtgccgcaacgagacgctctacctcctctacaaccgcgagggccagaccctcgtcgagaggtcatccacctgggtcaagaaggtcatctggtacctctccggccgcaaccagaccatcctccagcgcatgccccgcaccgcgtctaagccgtccgacggcaacgtccagatctccgtcgaggacgccaagatcttcggcgcccacatggtccccaagcagaccaagctcctccgcttcgtcgtcaacgacggcacccgctaccagatgtgcgtcatgaagctcgagtcctgggcccacgtcttccgcgactactccgtctccttccaggtccgcctcaccttcaccgaggccaataaccagacttacaccttctgcacccaccccaacctcatcgtc

SEQ ID NO: 142 - DNA, 642 bp, synthetic construct; identical to SEQ ID NO: 141
  misc_feature 1..642: note = Synthetic polynucleotide
  source 1..642: mol_type = other DNA

SEQ ID NO: 143 - DNA, 642 bp, synthetic construct
  misc_feature 1..642: note = Synthetic polynucleotide
  source 1..642: mol_type = other DNA
atgcttcggctcctcctaaggcaccacttccactgccttttgctttgcgccgtgtgggccaccccttgcttggccagcccctggagcaccctcaccgccaaccagaacccctcccctccctggtccaagctcacctactccaagccccacgacgccgccaccttctactgccccttcctctacccgtcccctccacgcagcccactccagttctccggcttccagcgcgtctccaccggccccgagtgccgcaacgaaacactctacctcctctacaaccgcgagggccagaccctcgtcgagcgctcctccacctgggtcaagaaggtcatctggtacctctccggccgcaaccagaccatcctccagcgcatgccccgcaccgcaagcaagccatccgacggcaacgtccagatctccgtcgaggacgccaagatcttcggcgcccacatggtccccaagcagaccaagctcctccgcttcgtcgtcaacgacggcacccgctaccagatgtgcgtcatgaagctcgagtcctgggcccacgtcttccgcgactactccgtctccttccaggtccgcctcaccttcaccgaggccaataatcagacatacaccttctgcacccaccccaacctcatcgtc

SEQ ID NO: 144 - DNA, 387 bp, synthetic construct
  misc_feature 1..387: note = Synthetic polynucleotide
  source 1..387: mol_type = other DNA
atgcggctgtgtcgggtgtggctgtctgtttgtctgtgcgccgtggtgctgggtcagtgccagcgggaaaccgcggaaaagaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaac

SEQ ID NO: 145 - DNA, 92 bp, synthetic construct
  misc_feature 1..92: note = Synthetic polynucleotide
  source 1..92: mol_type = other DNA
tcaagcttttggaccctcgtacagaagctaatacgactcactatagggaaataagagagaaaagaagagtaagaagaaatataagagccacc

SEQ ID NO: 146 - DNA, 47 bp, synthetic construct
  misc_feature 1..47: note = Synthetic polynucleotide
  source 1..47: mol_type = other DNA
gggaaataagagagaaaagaagagtaagaagaaatataagagccacc

SEQ ID NO: 147 - DNA, 119 bp, synthetic construct
  misc_feature 1..119: note = Synthetic polynucleotide
  source 1..119: mol_type = other DNA
tgataataggctggagcctcggtggccatgcttcttgccccttgggcctccccccagcccctcctccccttcctgcacccgtacccccgtggtctttgaataaagtctgagtgggcggc

SEQ ID NO: 148 - DNA, 16 bp, synthetic construct
  misc_feature 1..16: note = Synthetic polynucleotide
  source 1..16: mol_type = other DNA
ccgcgccaagaggagc

SEQ ID NO: 149 - DNA, 57 bp, synthetic construct
  misc_feature 1..57: note = Synthetic polynucleotide
  source 1..57: mol_type = other DNA
gctactaacttcagcctgctgaagcaggctggagacgtggaggagaaccctggacct

SEQ ID NO: 150 - DNA, 66 bp, synthetic construct
  misc_feature 1..66: note = Synthetic polynucleotide
  source 1..66: mol_type = other DNA
gtgaaacagactttgaattttgaccttctcaagttggcgggagacgtggagtccaaccctggacct

SEQ ID NO: 151 - DNA, 60 bp, synthetic construct
  misc_feature 1..60: note = Synthetic polynucleotide
  source 1..60: mol_type = other DNA
cagtgtactaattatgctctcttgaaattggctggagatgttgagagcaaccctggacct

SEQ ID NO: 152 - DNA, 54 bp, synthetic construct
  misc_feature 1..54: note = Synthetic polynucleotide
  source 1..54: mol_type = other DNA
gagggcagaggaagtctgctaacatgcggtgacgtcgaggagaatcctggacct

SEQ ID NO: 153 - protein, 22 aa, synthetic construct
  REGION 1..22: note = Synthetic polypeptide
GSGATNFSLLKQAGDVEENPGP

SEQ ID NO: 154 - protein, 21 aa, synthetic construct
  REGION 1..21: note = Synthetic polypeptide
GSGEGRGSLLTCGDVEENPGP

SEQ ID NO: 155 - protein, 23 aa, synthetic construct
  REGION 1..23: note = Synthetic polypeptide
GSGQCTNYALLKLAGDVESNPGP

SEQ ID NO: 156 - protein, 25 aa, synthetic construct
  REGION 1..25: note = Synthetic polypeptide
GSGVKQTLNFDLLKLAGDVESNPGP

SEQ ID NO: 157 - DNA, 2721 bp, synthetic construct
  misc_feature 1..2721: note = Synthetic polynucleotide
  source 1..2721: mol_type = other DNA
atggaaagcagaatttggtgcctcgtggtctgcgtgaacctctgtatcgtgtgcttaggcgccgcagtttcaagcagctccaccagaggtacgtcggctacccacagccatcactcaagtcacactacaagcgccgctcacagcagaagcggatctgtgagccagagggtgaccagctcccagaccgtgagccacggagtaaatgaaaccatctacaataccacattgaagtatggcgacgtcgtgggcgtgaacacgaccaaatacccctacagggtctgctctatggctcagggcactgacctgattcggtttgagagaaatatcgtctgcaccagcatgaagcccattaacgaggacctggatgagggcatcatggtggtatataaacgtaacattgtggcccacaccttcaaagtgagagtttaccagaaagtgctgaccttcagaagatcctacgcttacattcacacaacctacctgctgggctcaaacaccgaatacgtggcccctcccatgtgggaaatccaccacatcaactctcacagccagtgctacagctcttacagcagggttattgccggcaccgtcttcgtggcctaccaccgcgacagttatgagaacaagaccatgcagctgatgcctgacgactacagcaacacccactctaccagatacgtgaccgttaaggaccagtggcacagccggggctcaacctggctgtatcgggaaacttgtaacctgaattgcatggtgaccatcacaactgccagaagcaagtacccctatcacttcttcgccaccagcactggcgatgtggtggatatctctcccttctacaacggaaccaatcgcaacgcttcttactttggcgagaacgccgacaagttctttatctttcccaactacaccatcgtcagcgacttcggtagacccaattctgccctggaaactcatcgacttgtggcattcctggaaagggccgattccgtgatcagctgggacattcaggacgagaagaacgttacctgccagctcacattttgggaggccagcgagaggaccattaggagcgaggccgaggacagctaccacttttccagtgccaagatgacagccacatttctctctaagaagcaggaggttaacatgtccgacagcgccctggactgtgtcagagacgaggccatcaataagctgcagcagatcttcaacaccagctacaatcagacatatgagaagtacggcaacgtcagcgtcttcgagacaacaggcgggctggtcgtgttctggcagggaatcaaacagaagtccctggttgagctggagagactggcgaacaggagctctctgaatttgactcataacaggacgaagagatccaccgatggaaacaacgccacccacctgagcaatatggagagcgtccacaatctcgtctacgcccagctccaattcacctacgacaccctgaggggctatatcaaccgggccctggcccagatcgccgaggcatggtgcgtggaccagagacggaccctggaagtgttcaaggagctgtcaaagatcaacccttccgccatcctctccgccatatataataagcccatcgccgcaagattcatgggagatgtcctgggtctggctagctgcgttaccatcaaccagacatcagtgaaggttttgcgagacatgaatgtgaaggagtcacccggccgatgttacagccgcccagtcgtgatctttaacttcgccaattccagctacgtccaatacggccagctgggcgaggacaatgaaattctcctgggtaatcatagaaccgaggagtgccaactcccctcccttaagattttcatcgcaggcaatagcgcttatgagtacgttgactacttgtttaagagaatgatcgatctgagcagcatcagcacagtggactccatgattgcccttgatatcgatcccctggagaataccgactttagagtgctggagttatacagccagaaagagctgcgaagctccaacgtgttcgatctggaggaaattatgagggagtttaactcctacaagcagagagtgaagtacgtcgaagacaaagtggtggatccactgccgccttatcttaaaggcctcgacgatctgatgagcggactgggtgccgccggcaaagctgtgggcgttgccatcggagccgtgggcggggccgtggcctccgtggtggaaggcgtggctacctttctgaagaacccattcggcgcctttaccattatcctggtggccattgccgtggtgatcattacctatctcatctacactaggcagcggaggctgtgtacgcagcctctgcagaacctgtttccctacctggttagcgccgacggaacaacagtgacatctggctctaccaaggatacctctctgcaggcacctccttcttacgaggaatccgtgtacaactcgggaaggaaaggccccgggccaccttcatccgacgcctccacagctgccccgccatacactaacgagcaggcttaccagatgcttctcgccctggctagattggatgccgagcagcgcgcccaacagaacggcaccgacagcctggacggccggacaggcacccaggacaaagggcagaagcccaatctgcttgatagactgaggcaccggaagaacgggtacaggcatcttaaggacagcgacgaggaggagaacgtc

SEQ ID NO: 158 - RNA, 2437 nt, synthetic construct
  misc_feature 1..2437: note = Synthetic polynucleotide
  source 1..2437: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgctgataataggctggagcctcggtggccatgcttcttgccccttgggcctccccccagcccctcctccccttcctgcacccgtacccccgtggtctttgaataaagtctgagtgggcggc

SEQ ID NO: 159 - RNA, 2464 nt, synthetic construct
  misc_feature 1..2464: note = Synthetic polynucleotide
  source 1..2464: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgcgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 160 - RNA, 1045 nt, synthetic construct
  misc_feature 1..1045: note = Synthetic polynuceotide
  source 1..1045: mol_type = other RNA
tcaagcttttggaccctcgtacagaagctaatacgactcactatagggaaataagagagaaaagaagagtaagaagaaatataagagccaccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 161 - RNA, 1072 nt, synthetic construct
  misc_feature 1..1072: note = Synthetic polynucleotide
  source 1..1072: mol_type = other RNA
tcaagcttttggaccctcgtacagaagctaatacgactcactatagggaaataagagagaaaagaagagtaagaagaaatataagagccaccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgcgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 162 - RNA, 2932 nt, synthetic construct
  misc_feature 1..2932: note = Synthetic polynucleotide
  source 1..2932: mol_type = other RNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctgataataggctggagcctcggtggccatgcttcttgccccttgggcctccccccagcccctcctccccttcctgcacccgtacccccgtggtctttgaataaagtctgagtgggcggc

SEQ ID NO: 163 - DNA, 2956 bp, synthetic construct; identical to SEQ ID NO: 6
  misc_feature 1..2956: note = Synthetic polynucleotide
  source 1..2956: mol_type = other DNA
  modified_base order(7,8,9,10,17,20,29,32,38,42,44,52,70,80,82,94,99,106,108,111,115,118,121,123,127,128,132,133,135,137,139,142,144,146,148,152,155,160,161,162,165,168,170,171,173,176,179,185,186,188,191,194,200,206,207,210,212,215,222,224,227,230,234,236,240,245,246,250,252,254,262,266,267,269,270,280,287,290,292,293,304,306,314,319,324,332,334,337,343,347,357,363,365,370,372,374,375,377,379,389,395,397,398,400,401,405,406,407,413,416,418,421,423,429,433,442,446,454,466,469,472,475,477,490,493,504,505,506,511,517,519,529,530,531,532,537,538,539,542,545,549,554,555,559,569,570,572,574,577,594,598,605,608,610,612,619,620,623,626,628,641,645,648,653,654,657,667,668,670,682,683,684,685,688,692,693,695,698,708,710,724,729,730,733,743,744,746,747,761,767,768,772,778,785,789,810,814,816,818,821,828,830,833,835,839,840,842,844,847,853,857,860,867,873,875,878,879,881,884,885,886,887,888,889,896,897,905,910,913,914,919,920,921,923,926,927,928,930,944,950,957,960,961,962,981,982,983,984,985,988,989,990,991,992,999,1004,1006,1009,1011,1017,1018,1019,1031,1032,1034,1038,1039,1053,1054,1057,1061,1062,1063,1064,1066,1067,1073,1080,1084,1087,1089,1092,1097,1099,1115,1117,1121,1122,1124,1129,1133,1134,1135,1137,1146,1159,1160,1163,1164,1179,1182,1184,1188,1189,1190,1191,1193,1194,1196,1204,1214,1215,1216,1218,1219,1221,1223,1237,1243,1245,1251,1253,1258,1263,1267,1271,1274,1280,1282,1286,1290,1291,1300,1301,1302,1303,1307,1310,1311,1314,1319,1326,1328,1335,1337,1345,1347,1351,1353,1354,1355,1364,1367,1370,1371,1372,1375,1378,1380,1381,1383,1391,1393,1404,1406,1408,1411,1417,1424,1425,1426,1437,1442,1444,1448,1450,1451,1454,1457,1460,1475,1481,1490,1496,1499,1500,1501,1503,1510,1515,1519,1526,1528,1531,1533,1543,1548,1549,1554,1556,1563,1564,1571,1572,1576,1588,1597,1608,1611,1613,1615,1619,1633,1639,1641,1642,1651,1660,1668,1675,1676,1678,1680,1687,1688,1689,1702,1703,1712,1713,1714,1717,1721,1724,1726,1728,1729,1735,1743,1747,1753,1768,1774,1777,1781,1784,1786,1792,1800,1812,1815,1818,1828,1831,1834,1836,1837,1838,1841,1842,1843,1854,1857,1861,1866,1871,1876,1894,1897,1899,1900,1916,1923,1925,1930,1931,1939,1945,1947,1948,1951,1962,1968,1974,1978,1983,1987,1989,1990,1999,2002,2003,2008,2015,2017,2019,2026,2035,2038,2044,2048,2050,2059,2066,2073,2074,2080,2083,2089,2090,2091,2094,2107,2111,2112,2122,2123,2124,2125,2126,2131,2140,2143,2151,2152,2157,2160,2173,2178,2182,2194,2197,2206,2214,2218,2225,2227,2236,2239,2248,2269,2270,2275,2281,2282,2290,2294,2302,2307,2311,2314,2323,2324,2331,2332,2335,2346,2347,2355,2356,2362,2365,2368,2371,2377,2381,2383,2386,2389,2390,2392,2396,2397,2399,2400,2401,2404,2406,2408,2411,2423,2424,2425,2427,2440,2449,2451,2452,2453,2457,2459,2461,2464,2466,2485,2490,2511,2514,2515,2522,2528,2529,2532,2543,2545,2546,2547,2549,2552,2553,2555,2558,2580,2583,2585,2588,2592,2603,2609,2610,2627,2628,2635,2638,2639,2641,2647,2654,2656,2687,2693,2694,2696,2697,2698,2711,2743,2746,2755,2762,2775,2784,2785,2793,2795,2812,2816,2817,2828,2834,2838,2841,2844,2849,2856,2860,2866,2869,2870,2872,2873,2879,2880,2886,2899,2902,2907,2908,2911,2919,2927,2930,2932,2933,2934,2938,2943,2945,2949): mod_base = OTHER; note = uracil

SEQ ID NO: 164 - RNA, 2356 nt, synthetic construct
  misc_feature 1..2356: note = Synthetic polynucleotide
  source 1..2356: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgactgataataggctggagcctcggtggccatgcttcttgccccttgggcctccccccagcccctcctccccttcctgcacccgtacccccgtggtctttgaataaagtctgagtgggcggc

SEQ ID NO: 165 - RNA, 2383 nt, synthetic construct
  misc_feature 1..2383: note = Synthetic polynucleotide
  source 1..2383: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 166 - RNA, 2377 nt, synthetic construct
  misc_feature 1..2377: note = Synthetic polynucleotide
  source 1..2377: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgaccaccatcaccaccatcactgatgataataggctggagcctcggtggccatgcttcttgccccttgggcctccccccagcccctcctccccttcctgcacccgtacccccgtggtctttgaataaagtctgagtgggcggc

SEQ ID NO: 167 - RNA, 2287 nt, synthetic construct
  misc_feature 1..2287: note = Synthetic polynucleotide
  source 1..2287: mol_type = other RNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagtgataataggctggagcctcggtggccatgcttcttgccccttgggcctccccccagcccctcctccccttcctgcacccgtacccccgtggtctttgaataaagtctgagtgggcggc

SEQ ID NO: 168 - RNA, 2311 nt, synthetic construct
  misc_feature 1..2311: note = Synthetic polynucleotide
  source 1..2311: mol_type = other RNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcaggattacaaggacgatgacgataagtgataataggctggagcctcggtggccatgcttcttgccccttgggcctccccccagcccctcctccccttcctgcacccgtacccccgtggtctttgaataaagtctgagtgggcggc

SEQ ID NO: 169 - RNA, 2305 nt, synthetic construct
  misc_feature 1..2305: note = Synthetic polynucleotide
  source 1..2305: mol_type = other RNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcaccatcaccaccatcactgataataggctggagcctcggtggccatgcttcttgccccttgggcctccccccagcccctcctccccttcctgcacccgtacccccgtggtctttgaataaagtctgagtgggcggc

SEQ ID NO: 170 - RNA, 724 nt, synthetic construct
  misc_feature 1..724: note = Synthetic polynucleotide
  source 1..724: mol_type = other RNA
tcaagcttttggaccctcgtacagaagctaatacgactcactatagggaaataagagagaaaagaagagtaagaagaaatataagagccaccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagtgataataggctggagcctcggtggccatgcttcttgccccttgggcctccccccagcccctcctccccttcctgcacccgtacccccgtggtctttgaataaagtctgagtgggcggc

SEQ ID NO: 171 - RNA, 748 nt, synthetic construct
  misc_feature 1..748: note = Synthetic polynucleotide
  source 1..748: mol_type = other RNA
tcaagcttttggaccctcgtacagaagctaatacgactcactatagggaaataagagagaaaagaagagtaagaagaaatataagagccaccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcaggattacaaggacgatgacgataagtgataataggctggagcctcggtggccatgcttcttgccccttgggcctccccccagcccctcctccccttcctgcacccgtacccccgtggtctttgaataaagtctgagtgggcggc

SEQ ID NO: 172 - RNA, 853 nt, synthetic construct
  misc_feature 1..853: note = Synthetic polynucleotide
  source 1..853: mol_type = other RNA
tcaagcttttggaccctcgtacagaagctaatacgactcactatagggaaataagagagaaaagaagagtaagaagaaatataagagccaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 173 - RNA, 880 nt, synthetic construct
  misc_feature 1..880: note = Synthetic polynucleotide
  source 1..880: mol_type = other RNA
tcaagcttttggaccctcgtacagaagctaatacgactcactatagggaaataagagagaaaagaagagtaagaagaaatataagagccaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 174 - RNA, 598 nt, synthetic construct
  misc_feature 1..598: note = Synthetic polynucleotide
  source 1..598: mol_type = other RNA
tcaagcttttggaccctcgtacagaagctaatacgactcactatagggaaataagagagaaaagaagagtaagaagaaatataagagccaccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 175 - RNA, 625 nt, synthetic construct
  misc_feature 1..625: note = Synthetic polynucleotide
  source 1..625: mol_type = other RNA
tcaagcttttggaccctcgtacagaagctaatacgactcactatagggaaataagagagaaaagaagagtaagaagaaatataagagccaccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaacgattacaaggacgatgacgataagtgatgataataggctggagcctcggtggccatgcttcttgccccttgggcctccccccagcccctcctccccttcctgcacccgtacccccgtggtctttgaataaagtctgagtgggcggc

SEQ ID NO: 176 - RNA, 2434 nt, synthetic construct
  misc_feature 1..2434: note = Synthetic polynucleotide
  source 1..2434: mol_type = other RNA
tcaagcttttggaccctcgtacagaagctaatacgactcactatagggacagacgagagagaagcacgccaattctgcctgcttaagccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgctgataataggctggagcctcggtggccatgcttcttgccccttgggcctccccccagcccctcctccccttcctgcacccgtacccccgtggtctttgaataaagtctgagtgggcggc

SEQ ID NO: 177 - RNA, 1044 nt, synthetic construct
  misc_feature 1..1044: note = Synthetic polynucleotide
  source 1..1044: mol_type = other RNA
tcaagcttttggaccctcgtacagaagctaatacgactcactatagggcttaagcaggcagaattggcccttagcctgtaccagccgaaccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 178 - RNA, 1045 nt, synthetic construct
  misc_feature 1..1045: note = Synthetic polynucleotide
  source 1..1045: mol_type = other RNA
tcaagcttttggaccctcgtacagaagctaatacgactcactatagggtggctcttatatttcttcttactcttcttttctctcttatttccatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 179 - RNA, 722 nt, synthetic construct
  misc_feature 1..722: note = Synthetic polynucleotide
  source 1..722: mol_type = other RNA
tcaagcttttggaccctcgtacagaagctaatacgactcactatagggttcggctggtacaggctaaccagaagacagataagagcctccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagtgataataggctggagcctcggtggccatgcttcttgccccttgggcctccccccagcccctcctccccttcctgcacccgtacccccgtggtctttgaataaagtctgagtgggcggc

SEQ ID NO: 180 - RNA, 854 nt, synthetic construct
  misc_feature 1..854: note = Synthetic polynucleotide
  source 1..854: mol_type = other RNA
tcaagcttttggaccctcgtacagaagctaatacgactcactatagggaggctcttatctgtcttctcagtccgaattcgaagtacggctaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 181 - RNA, 853 nt, synthetic construct
  misc_feature 1..853: note = Synthetic polynucleotide
  source 1..853: mol_type = other RNA
tcaagcttttggaccctcgtacagaagctaatacgactcactatagggtggctcttatatttcttcttagtccgaattcgaagtacggctacatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 182 - RNA, 597 nt, synthetic construct
  misc_feature 1..597: note = Synthetic polynucleotide
  source 1..597: mol_type = other RNA
tcaagcttttggaccctcgtacagaagctaatacgactcactatagggtagccgtacttcgaattcggacaagcttctctctcgtctgtccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 183 - RNA, 597 nt, synthetic construct
  misc_feature 1..597: note = Synthetic polynucleotide
  source 1..597: mol_type = other RNA
tcaagcttttggaccctcgtacagaagctaatacgactcactatagggtagccgtacttcgaattcggactttcttttctctcttatttccatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 184 - RNA, 3364 nt, synthetic construct
  misc_feature 1..3364: note = Synthetic polynucleotide
  source 1..3364: mol_type = other RNA
tcaagcttttggaccctcgtacagaagctaatacgactcactatagggaaataagagagaaaagaagagtaagaagaaatataagagccaccatggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaagagcccatgtcgatctatgtgtacgcgctgccgctcaagatgctgaacatccccagcatcaacgtgcaccactacccgtcggcggccgagcgcaaacaccgacacctgcccgtagccgacgctgttattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctaacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcaaatcttcctggaggtacaagcgatacgcgagaccgtggaactgcgtcagtacgatcccgtggctgcgctcttctttttcgatatcgacttgttgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgtaagacgccccgcgtcaccggcggcggcgccatggcgagcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggtgagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcattgcagaacttgcctcagtgctctcctgatgagattatgtcttatgcccagaaaatctttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctttgagagtctgctctcctagtgtggatgacctacgggccatcgccgaggagtcagatgaggaagaggctattgtagcctacactttggccaccgctggtgccagctcctctgattctctggtgtcacctccagagtcccctgtacccgcgactatccctctgtcctcagtaattgtggctgagaacagtgatcaggaagaaagtgaacagagtgatgaggaacaggaggagggtgctcaggaggagcgggaggacactgtgtctgtcaagtctgagccagtgtctgagatagaggaagttgcctcagaggaagaggaggatggtgctgaggaacccaccgcctctggaggcaagagcacccaccctatggtgactagaagcaaggctgaccagtgataataggctggagcctcggtggccatgcttcttgccccttgggcctccccccagcccctcctccccttcctgcacccgtacccccgtggtctttgaataaagtctgagtgggcggc

SEQ ID NO: 185 - RNA, 3364 nt, synthetic construct; identical to SEQ ID NO: 184
  misc_feature 1..3364: note = Synthetic polynucleotide
  source 1..3364: mol_type = other RNA

SEQ ID NO: 186 - RNA, 3352 nt, synthetic construct
  misc_feature 1..3352: note = Synthetic polynucleotide
  source 1..3352: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgccgcgccaagaggagcggaagcggagctactaacttcagcctgctgaagcaggctggagacgtggaggagaaccctggacctatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctgataataggctggagcctcggtggccatgcttcttgccccttgggcctccccccagcccctcctccccttcctgcacccgtacccccgtggtctttgaataaagtctgagtgggcggc

SEQ ID NO: 187 - RNA, 1924 nt, synthetic construct
  misc_feature 1..1924: note = Synthetic polynucleotide
  source 1..1924: mol_type = other RNA
tcaagcttttggaccctcgtacagaagctaatacgactcactatagggaaataagagagaaaagaagagtaagaagaaatataagagccaccatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagcgcgccaagaggagcggaagcggagctactaacttcagcctgctgaagcaggctggagacgtggaggagaaccctggacctatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttcgcgccaagaggagcggaagcggagtgaaacagactttgaattttgaccttctcaagttggcgggagacgtggagtccaaccctggacctatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 188 - RNA, 5146 nt, synthetic construct
  misc_feature 1..5146: note = Synthetic polynucleotide
  source 1..5146: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgccgcgccaagaggagcggaagcggagctactaacttcagcctgctgaagcaggctggagacgtggaggagaaccctggacctatgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgccgcgccaagaggagcggaagcggagtgaaacagactttgaattttgaccttctcaagttggcgggagacgtggagtccaaccctggacctatgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagcgcgccaagaggagcggaagcggacagtgtactaattatgctctcttgaaattggctggagatgttgagagcaaccctggacctatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttcgcgccaagaggagcggaagcggagagggcagaggaagtctgctaacatgcggtgacgtcgaggagaatcctggacctatgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 189 - RNA, 2437 nt, synthetic construct
  misc_feature 1..2437: note = Synthetic polynucleotide
  source 1..2437: mol_type = other RNA
tcaagcttttggaccctcgtacagaagctaatacgactcactatagggaaataagagagaaaagaagagtaagaagaaatataagagccaccatgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaacttcttccaaagctataatcaatactatgtattccatatgcctcgatgtctctttgcgggtcctctggcggagcagtttctgaaccaggtagatctgaccgaaaccctggaaagataccaacagagacttaacacttacgcgctggtatccaaagacctggccagctaccgatctttctcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgagaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaagaacggtacggtactagaagtaactgacgtcgtcgtggacgccaccgacagtcgtctcctcatgatgtccgtctacgcgctatcggccatcatcggcatctatctgctctaccgcatgctcaagacatgctgataataggctggagcctcggtggccatgcttcttgccccttgggcctccccccagcccctcctccccttcctgcacccgtacccccgtggtctttgaataaagtctgagtgggcggc

SEQ ID NO: 190 - RNA, 1045 nt, synthetic construct; identical to SEQ ID NO: 160
  misc_feature 1..1045: note = Synthetic polynucleotide
  source 1..1045: mol_type = other RNA

SEQ ID NO: 191 - RNA, 724 nt, synthetic construct; identical to SEQ ID NO: 170
  misc_feature 1..724: note = Synthetic polynucleotide
  source 1..724: mol_type = other RNA

SEQ ID NO: 192 - RNA, 846 nt, synthetic construct
  misc_feature 1..846: note = Synthetic polynucleotide
  source 1..846: mol_type = other RNA
tcaagcttttggaccctcgtacagaagctaatacgactcactatagggaaataagagagaaaagaagagtaagaagaaatataagagccaccatgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcgctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttgataataggctggagcctcggtggccatgcttcttgccccttgggcctccccccagcccctcctccccttcctgcacccgtacccccgtggtctttgaataaagtctgagtgggcggc

SEQ ID NO: 193 - RNA, 598 nt, synthetic construct
  misc_feature 1..598: note = Synthetic polynucleotide
  source 1..598: mol_type = other RNA
tcaagcttttggaccctcgtacagaagctaatacgactcactatagggaaataagagagaaaagaagagtaagaagaaatataagagccaccatgcggctgtgtcgggtgtggctgtctgtttgtctgtgcgccgtggtgctgggtcagtgccagcgggaaaccgcggaaaagaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactgataataggctggagcctcggtggccatgcttcttgccccttgggcctccccccagcccctcctccccttcctgcacccgtacccccgtggtctttgaataaagtctgagtgggcggc

SEQ ID NO: 194 - RNA, 2933 nt, synthetic construct
  misc_feature 1..2933: note = Synthetic polynucleotide
  source 1..2933: mol_type = other RNA
tcaagcttttggaccctcgtacagaagctaatacgactcactatagggaaataagagagaaaagaagagtaagaagaaatataagagccaccatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtcttgataataggctggagcctcggtggccatgcttcttgccccttgggcctccccccagcccctcctccccttcctgcacccgtacccccgtggtctttgaataaagtctgagtgggcggc

(19) United States
(12) Patent Application Publication
(10) Pub. No.: US 2025/0228932 A1
(43) Pub. Date: Jul. 17, 2025

(54) HUMAN CYTOMEGALOVIRUS VACCINE

(71) Applicant: ModernaTX, Inc., Cambridge, MA (US)

(72) Inventors: Giuseppe Ciaramella, Cambridge, MA (US); Shinu John, Cambridge, MA (US)

(73) Assignee: ModernaTX, Inc., Cambridge, MA (US)

(21) Appl. No.: 18/926,517

(22) Filed: Oct. 25, 2024

Related U.S. Application Data
(63) Continuation of application No. 17/839,401, filed on Jun. 13, 2022, now abandoned.
Application No. 17/839,401 is a continuation of application No. 17/548,172, filed on Dec. 10, 2021, now Pat. No. 11,541,113.
Application No. 17/548,172 is a continuation of application No. 16/864,566, filed on May 1, 2020, now Pat. No. 11,197,927.
Application No. 16/864,566 is a continuation of application No. 16/389,545, filed on Apr. 19, 2019, now Pat. No. 10,695,419.
Application No. 16/389,545 is a continuation of application No. PCT/US2017/057748, filed on Oct. 20, 2017.
(60) Provisional application No. 62/548,184, filed on Aug. 21, 2017.
(60) Provisional application No. 62/490,541, filed on Apr. 26, 2017.
(60) Provisional application No. 62/490,510, filed on Apr. 26, 2017.
(60) Provisional application No. 62/411,381, filed on Oct. 21, 2016.

(51) Int. Cl. A61K 39/245; A61K 39/00; A61P 31/22

(52) U.S. Cl. CPC A61K 39/245; A61P 31/22; A61K 2039/53; A61K 2039/6018; C12N 2710/16134; C12N 2710/16171

(57) ABSTRACT

The disclosure relates to HCMV ribonucleic acid (RNA) vaccines, as well as methods of using the vaccines and compositions comprising the vaccines.

### RELATED APPLICATIONS

This application is a continuation of U.S. application Ser. No. 16/389,545, filed Apr. 19, 2019, entitled “Human Cytomegalovirus Vaccine,” which is a continuation of International Application No. PCT/US2017/057748, filed Oct. 20, 2017, which claims the benefit under 35 U.S.C. § 119(e) of U.S. Provisional Application Ser. No. 62/548,184, filed Aug. 21, 2017, entitled “Human Cytomegalovirus Vaccine,” U.S. Provisional Application Ser. No. 62/490,510, filed Apr. 26, 2017, entitled “Human Cytomegalovirus Vaccine,” U.S. Provisional Application Ser. No. 62/490,541, filed Apr. 26, 2017, entitled “Human Cytomegalovirus Vaccine,” and U.S. Provisional Application No. 62/411,381, filed Oct. 21, 2016, entitled “Human Cytomegalovirus Vaccine,” each of which is incorporated by reference herein in its entirety.

### BACKGROUND

Human cytomegalovirus (HCMV) is a genus of viruses in the order Herpesvirales, in the family Herpesviridae, in the subfamily Betaherpesvirinae. There are currently eight species in this genus, which have been identified and classified for different mammals, including humans, monkeys, and rodents. The most studied genus is human cytomegalovirus, also known as human herpesvirus 5 (HHV-5), which is widely distributed in the human population. Diseases associated with HHV-5 include mononucleosis and pneumonias. All herpesviruses share a characteristic ability to remain latent within the body over long periods of time. Although they may be found throughout the body, CMV infections are frequently associated with the salivary glands in humans and other mammals. Other CMV viruses are found in several mammal species, but species isolated from animals differ from HCMV in terms of genomic structure, and have not been reported to cause human disease.

HCMV is endemic in most parts of the world. It is a ubiquitous large enveloped virus that infects 50 to 100% of the adult population worldwide. Although generally asymptomatic in immunocompetent hosts, HCMV infection is a major cause of morbidity and mortality in immunocompromised persons, such as infants following congenital or neonatal infections, transplant recipients, or AIDS patients.

Primary infection normally results in subclinical disease after which the virus becomes latent, retaining the capacity to reactivate at a later time. The virus is transmitted through body fluids, such as blood, saliva, urine, semen and breast milk. In particular, individuals with undeveloped or compromised immunity are highly sensitive to infection by HCMV. It is estimated that at least 60% of the US population has been exposed to CMV, with a prevalence of more than 90% in high-risk groups (e.g., unborn babies whose mothers become infected with CMV during the pregnancy or people with HIV).

In healthy individuals, HCMV typically causes an asymptomatic infection or produces mild, flulike symptoms. However, among two populations, HCMV is responsible for serious medical conditions. First, HCMV is a major cause of congenital defects in newborns infected in utero. Among congenitally infected newborns, 5-10% have major clinical symptoms at birth, such as microcephaly, intracranial calcifications, and hepatitis, as well as cytomegalic inclusion disease, which affects many tissues and organs including the central nervous system, liver, and retina and can lead to multi-organ failure and death. Other infants may be asymptomatic at birth, but later develop hearing loss or central nervous system abnormalities causing, in particular, poor intellectual performance and mental retardation. These pathologies are due in part to the ability of HCMV to enter and replicate in diverse cell types including epithelial cells, endothelial cells, smooth muscle cells, fibroblasts, neurons, and monocytes/macrophages.

The second population at risk are immunocompromised patients, such as those suffering from HIV infection and those undergoing transplantations. In this situation, the virus becomes an opportunistic pathogen and causes severe disease with high morbidity and mortality. The clinical disease causes a variety of symptoms including fever, pneumonia, hepatitis, encephalitis, myelitis, colitis, uveitis, retinitis, and neuropathy. Rarer manifestations of HCMV infections in immunocompetent individuals include Guillain-Barre syndrome, meningoencephalitis, pericarditis, myocarditis, thrombocytopenia, and hemolytic anemia. Moreover. HCMV infection increases the risk of organ graft loss through transplant vascular sclerosis and restenosis, and may increase atherosclerosis in transplant patients as well as in the general population. It is estimated that HCMV infection causes clinical disease in 75% of patients in the first year after transplantation.

There is currently no approved HCMV vaccine. Two candidate vaccines, Towne and gB/MF59, have completed phase II efficacy trials. The Towne vaccine appears protective against both infection and disease caused by challenge with pathogenic Toledo strain and also appears to be effective in preventing severe post-transplantation CMV disease. However, in a small phase II clinical trial, a low dose of Towne vaccine failed to show protection against infection of seronegative mothers who had children actively shedding CMV.

The gB/MF59 vaccine is a protein subunit vaccine comprised of a transmembrane-deleted version of HCMV gB protein, which induces high levels of fibroblast entry neutralizing antibodies in humans and has been shown to be safe and well tolerated in both adults and toddlers. A recent phase II double-blind placebo-controlled trial of the gB/MF59 vaccine revealed a 50% efficacy in inducing sterilizing immunity. As this vaccine induces potent antibody responses but very weak T-cell responses, the partial efficacy provided by the vaccine is thought to be primarily antibody-mediated. While this HCMV vaccine is the first to show any protective efficacy, its 50% protection falls short of the 80-90% desired for most vaccines.

In addition, antibody therapy has been used to control HCMV infection in immunocompromised individuals and to reduce the pathological consequences of maternal-fetal transmission, although such therapy is usually not sufficient to eradicate the virus. HCMV immunoglobulins (Igs) have been administered to transplant patients in association with immunosuppressive treatments for prophylaxis of HCMV disease with mixed results. Antibody therapy has also been used to control brief infection and prevent disease in newborns. However, these products are plasma derivatives with relatively low potency and have to be administered by intravenous infusion at very high doses in order to deliver sufficient amounts of neutralizing antibodies.

HCMV is the leading viral cause of neurodevelopmental abnormality and other birth defects in children and the costs to society are substantial. Although antiviral therapy is available, the treatment with antiviral agents is imperfect and development of a CMV vaccine is the most promising strategy for preventing CMV infection. Given that the health and economic benefits of effective HCMV vaccines are significant, the US Institute of Medicine and US National Vaccine Program Office has categorized development of a CMV vaccine as a highest priority, but no candidate vaccine is under consideration for licensure.

### SUMMARY

In view of the lack of HCMV vaccines, there is a significant need for a vaccine that would be safe and effective in all patient populations to prevent and/or to treat HCMV infection. In particular, there is a need for a vaccine that would be safe and effective for immunocompromised, at-risk pregnant women, and infant patients to prevent or to reduce the severity and/or duration of HCMV. Provided herein is a ribonucleic acid (RNA) vaccine that builds on the knowledge that RNA (e.g., messenger RNA (mRNA)) can safely direct the body's cellular machinery to produce nearly any protein of interest, from native proteins to antibodies and other entirely novel protein constructs that can have therapeutic activity inside and outside of cells. The HCMV RNA vaccines of the present disclosure may be used to induce a balanced immune response against human cytomegalovirus comprising both cellular and humoral immunity, without many of the risks associated with DNA or attenuated virus vaccination.

The RNA vaccines may be utilized in various settings depending on the prevalence of the infection or the degree or level of unmet medical need. The RNA vaccines may be utilized to treat and/or prevent a HCMV of various genotypes, strains, and isolates. The RNA vaccines have superior properties in that they produce much larger antibody titers and produce responses earlier than commercially available anti-viral therapeutic treatments. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines which are manufactured ex vivo and may trigger unwanted cellular responses, the RNA vaccines are presented to the cellular system in a more native fashion.

Various human cytomegalovirus amino acid sequences encompasses by the present disclosure are provided in Tables 2, 6, 7, 8, and 9 below. RNA vaccines as provided herein may include at least one RNA polynucleotide encoding at least one of the HCMV proteins provided in Tables 2, 6, 7, 8 or 9, or a fragment, homolog (e.g., having at least 80%, 85%, 90%, 95%, 98% or 99% identity) or derivative thereof.

Some embodiments of the present disclosure provide HCMV vaccines that include at least one ribonucleic acid (RNA) polynucleotide having an open reading frame encoding at least one HCMV antigenic polypeptide or an immunogenic fragment or epitope thereof. Some embodiments of the present disclosure provide HCMV vaccines that include at least one RNA polynucleotide having an open reading frame encoding two or more HCMV antigenic polypeptides or an immunogenic fragment or epitope thereof. Some embodiments of the present disclosure provide HCMV vaccines that include two or more RNA polynucleotides having an open reading frame encoding two or more HCMV antigenic polypeptides or immunogenic fragments or epitopes thereof. The one or more HCMV antigenic polypeptides may be encoded on a single RNA polynucleotide or may be encoded individually on multiple (e.g., two or more) RNA polynucleotides.

In some embodiments, an antigenic polypeptide is an HCMV glycoprotein. For example, a HCMV glycoprotein may be selected from HCMV gH, gL, gB, gO, gN, and gM and an immunogenic fragment or epitope thereof. In some embodiments, the antigenic polypeptide is a HCMV gH polypeptide. In some embodiments, the antigenic polypeptide is a HCMV gL polypeptide. In some embodiments, the antigenic polypeptide is a HCMV gB polypeptide. In some embodiments, the antigenic polypeptide is a HCMV gO polypeptide. In some embodiments, the antigenic polypeptide is a HCMV gN polypeptide. In some embodiments, the antigenic polypeptide is a HCMV gM polypeptide. In some embodiments, the HCMV glycoprotein is encoded by a nucleic acid sequence of SEQ ID NO: 1, SEQ ID NO:2, SEQ ID NO: 3, SEQ ID NO: 4, SEQ ID NO: 5, or SEQ ID NO:6.

In some embodiments, the HCMV glycoprotein is a variant gH polypeptide, a variant gL polypeptide, or a variant gB polypeptide. In some embodiments, the variant HCMV gH, gL, or gB polypeptide is a truncated polypeptide lacking one or more of the following domain sequences: (1) the hydrophobic membrane proximal domain, (2) the transmembrane domain, and (3) the cytoplasmic domain. In some embodiments, the truncated HCMV gH, gL, or gB polypeptide lacks the hydrophobic membrane proximal domain, the transmembrane domain, and the cytoplasmic domain. In some embodiments, the truncated HCMV gH, gL, or gB polypeptide comprises only the ectodomain sequence. In some embodiments, the HCMV truncated glycoprotein is encoded by a nucleic acid sequence of SEQ ID NO: 7, SEQ ID NO:8, SEQ ID NO: 9, SEQ ID NO: 10, SEQ ID NO: 11, or SEQ ID NO:12.

In some embodiments, an antigenic polypeptide is an HCMV protein selected from UL83, UL123, UL128, UL130 and UL131A or an immunogenic fragment or epitope thereof. In some embodiments, the antigenic polypeptide is a HCMV UL83 polypeptide. In some embodiments, the antigenic polypeptide is a HCMV UL123 polypeptide. In some embodiments, the antigenic polypeptide is a HCMV UL128 polypeptide. In some embodiments, the antigenic polypeptide is a HCMV UL130 polypeptide. In some embodiments, the antigenic polypeptide is a HCMV UL131A polypeptide. In some embodiments, the HCMV protein is encoded by a nucleic acid sequence of SEQ ID NO: 13, SEQ ID NO:14, SEQ ID NO: 15, SEQ ID NO: 16, SEQ ID NO: 17, or SEQ ID NO:18.

In some embodiments, the antigenic polypeptide comprises two or more HCMV proteins, fragments, or epitopes thereof. In some embodiments, the antigenic polypeptide comprises two or more glycoproteins, fragments, or epitopes thereof. In some embodiments, the antigenic polypeptide comprises at least one HCMV glycoprotein, fragment or epitope thereof and at least one other HCMV protein, fragment or epitope thereof. In some embodiments, the two or more HCMV polypeptides are encoded by a single RNA polynucleotide. In some embodiments, the two or more HCMV polypeptides are encoded by two or more RNA polynucleotides, for example, each HCMV polypeptide is encoded by a separate RNA polynucleotide. In some embodiments, the two or more HCMV glycoproteins can be any combination of HCMV gH, gL, gB, gO, gN, and gM polypeptides or immunogenic fragments or epitopes thereof. In some embodiments, the two or more glycoproteins can be any combination of HCMV gB and one or more HCMV polypeptides selected from gH, gL, gO, gN, and gM polypeptides or immunogenic fragments or epitopes thereof. In some embodiments, the two or more glycoproteins can be any combination of HCMV gH and one or more HCMV polypeptides selected from gL, gO, gN, and gM polypeptides or immunogenic fragments or epitopes thereof. In some embodiments, the two or more glycoproteins can be any combination of HCMV gL and one or more HCMV polypeptides selected from gB, gH, gO, gN, and gM polypeptides or immunogenic fragments or epitopes thereof. In some embodiments, the two or more HCMV glycoproteins are gB and gH. In some embodiments, the two or more HCMV glycoproteins are gB and gL. In some embodiments, the two or more HCMV glycoproteins are gH and gL. In some embodiments, the two or more HCMV glycoproteins are gB, gL, and gH. In some embodiments, the two or more HCMV proteins can be any combination of HCMV UL83, UL123, UL128, UL130, and UL131A polypeptides or immunogenic fragments or epitopes thereof. In some embodiments, the two or more HCMV glycoproteins are UL123 and UL130. In some embodiments, the two or more HCMV glycoproteins are UL123 and 131A. In some embodiments, the two or more HCMV glycoproteins are UL130 and 131A. In some embodiments, the two or more HCMV glycoproteins are UL 128, UL130 and 131A. In some embodiments, the two or more HCMV proteins can be any combination of HCMV gB, gH, gL, gO, gM, gN, UL83, UL123, UL128, UL130, and UL131A polypeptides or immunogenic fragments or epitopes thereof. In some embodiments, the two or more glycoproteins can be any combination of HCMV gH and one or more HCMV polypeptides selected from gL, UL128, UL130, and UL131A polypeptides or immunogenic fragments or epitopes thereof. In some embodiments, the two or more glycoproteins can be any combination of HCMV gL and one or more HCMV polypeptides selected from gH, UL128, UL130, and UL131A polypeptides or immunogenic fragments or epitopes thereof. In some embodiments, the two or more HCMV glycoproteins are gL, gH, UL 128, UL130 and 131A. In any of these embodiments in which the vaccine comprises two or more HCMV proteins, the HCMV gH may be a variant gH, such as any of the variant HCMV gH glycoproteins disclosed herein, for example, any of the variant HCMV gH disclosed in the preceding paragraphs and in the Examples. In any of these embodiments in which the vaccine comprises two or more HCMV proteins, the HCMV gB may be a variant gB, such as any of the variant HCMV gB glycoproteins disclosed herein, for example, any of the variant HCMV gB disclosed in the preceding paragraphs and in the Examples. In any of these embodiments in which the vaccine comprises two or more HCMV gL proteins, the HCMV gL may be a variant gL, such as any of the variant HCMV gL glycoproteins disclosed herein, for example, any of the variant HCMV gL disclosed in the preceding paragraphs and in the Examples.

In certain embodiments in which the HCMV vaccine includes two or more RNA polynucleotides having an open reading frame encoding two or more HCMV antigenic polypeptides or an immunogenic fragment or epitope thereof (either encoded by a single RNA polynucleotide or encoded by two or more RNA polynucleotides, for example, each protein encoded by a separate RNA polynucleotide), the two or more HCMV proteins are a variant gB, for example, any of the variant gB polypeptides disclosed herein in the preceding paragraphs, and a HCMV protein selected from gH, gL, gO, gM, gN, UL128, UL130, and UL131A polypeptides or immunogenic fragments or epitopes thereof. In some embodiments, the two or more HCMV proteins are a variant gH, for example, any of the variant gH polypeptides disclosed herein in the preceding paragraphs, and a HCMV protein selected from gH, gL, gO, gM, gN, UL 128, UL130, and UL131A polypeptides or immunogenic fragments or epitopes thereof. In some embodiments, the two or more HCMV proteins are a variant gH, for example, any of the variant gH polypeptides disclosed herein in the preceding paragraphs, and a HCMV protein selected from gH, gL, gO, gM, gN, UL128, UL130, and UL131A polypeptides or immunogenic fragments or epitopes thereof. In some embodiments in which the variant HCMV proteins are variant HCMV gB, variant HCMV gL, and variant HCMV gH, the variant HCMV polypeptide is a truncated polypeptide selected from the following truncated polypeptides: lacks the hydrophobic membrane proximal domain; lacks the transmembrane domain; lacks the cytoplasmic domain; lacks two or more of the hydrophobic membrane proximal, transmembrane, and cytoplasmic domains; and comprises only the ectodomain.

In some embodiments, the HCMV vaccine includes multimeric RNA polynucleotides having an open reading frame encoding at least one HCMV antigenic polypeptide or an immunogenic fragment or epitope thereof. Some embodiments of the present disclosure provide HCMV vaccines that include at least one ribonucleic acid (RNA) polynucleotide having an open reading frame encoding at least one HCMV antigenic polypeptide or an immunogenic fragment or epitope thereof, wherein the 5′UTR of the RNA polynucleotide comprises a patterned UTR. In some embodiments, the patterned UTR has a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level. In some embodiments, the 5′UTR of the RNA polynucleotide (e.g., a first nucleic acid) has regions of complementarity with a UTR of another RNA polynucleotide (a second nucleic acid). For example, UTR nucleotide sequences of two polynucleotides sought to be joined (e.g., in a multimeric molecule) can be modified to include a region of complementarity such that the two UTRs hybridize to form a multimeric molecule.

In some embodiments, the 5′UTR of an RNA polynucleotide encoding an HCMV antigenic polypeptide is modified to allow the formation of a multimeric sequence. In some embodiments, the 5′UTR of an RNA polynucleotide encoding an HCMV protein selected from UL128, UL130, UL131A1 is modified to allow the formation of a multimeric sequence. In some embodiments, the 5′UTR of an RNA polynucleotide encoding an HCMV glycoprotein is modified to allow the formation of a multimeric sequence. In some embodiments, the 5′UTR of an RNA polynucleotide encoding an HCMV glycoprotein selected from gH, gL, gB, gO, gM, and gN is modified to allow the formation of a multimeric sequence. In any of these embodiments, the multimer may be a dimer, a trimer, pentamer, hexamer, heptamer, octamer nonamer, or decamer. Thus, in some embodiments, the 5′UTR of an RNA polynucleotide encoding an HCMV protein selected from gH, gL, gB, gO, gM, gN, UL128, UL130, and UL131A1 is modified to allow the formation of a dimer. In some embodiments, the 5′UTR of an RNA polynucleotide encoding an HCMV protein selected from gH, gL, gB, gO, gM, gN, UL128, UL130, and UL131A 1 is modified to allow the formation of a trimer. In some embodiments, the 5′UTR of an RNA polynucleotide encoding an HCMV protein selected from gH, gL, gB, gO, gM, gN, UL128, UL130, and UL131A1 is modified to allow the formation of a pentamer. Exemplary HCMV nucleic acids having modified 5′UTR sequence for the formation of a multimeric molecule (e.g., dimers, trimers, pentamers, etc) comprise SEQ ID Nos: 19-26.

In any of the above-described embodiments, the HCMV RNA polynucleotides may further comprise additional sequences, for example, one or more linker sequences or one or more sequence tags, such as FLAG-tag and histidine tag.

Some embodiments of the present disclosure provide HCMV vaccines that include at least one ribonucleic acid (RNA) polynucleotide having a single open reading frame encoding two or more (for example, two, three, four, five, or more) HCMV antigenic polypeptides or an immunogenic fragment or epitope thereof. Some embodiments of the present disclosure provide HCMV vaccines that include at least one ribonucleic acid (RNA) polynucleotide having more than one open reading frame, for example, two, three, four, five or more open reading frames encoding two, three, four, five or more HCMV antigenic polypeptides. In either of these embodiments, the at least one RNA polynucleotide may encode two or more HCMV antigenic polypeptides selected from gH, gB, gL, gO, gM, gN, UL83, UL123, UL128, UL130, UL131A, and fragments or epitopes thereof. In some embodiments, the at least one RNA polynucleotide encodes UL83 and UL123. In some embodiments, the at least one RNA polynucleotide encodes gH and gL. In some embodiments, the at least one RNA polynucleotide encodes UL128, UL130, and UL131A. In some embodiments, the at least one RNA polynucleotide encodes gH, gL, UL128, UL130, and UL131A. In some embodiments, in which the at least one RNA polynucleotide has a single open reading frame encoding two or more (for example, two, three, four, five, or more) HCMV antigenic polypeptides, the RNA polynucleotide further comprises additional sequence, for example, a linker sequence or a sequence that aids in the processing of the HCMV RNA transcripts or polypeptides, for example a cleavage site sequence. In some embodiments, the additional sequence may be a protease sequence, such as a furin sequence. In some embodiments, the additional sequence may be self-cleaving 2A peptide, such as a P2A, E2A, F2A, and T2A sequence. In some embodiments, the linker sequences and cleavage site sequences are interspersed between the sequences encoding HCMV polypeptides. In some embodiments, the RNA polynucleotide is encoded by SEQ ID NO: 27, SEQ ID NO: 28, SEQ ID NO: 29, SEQ ID NO: 30 or SEQ ID NO: 31.

In some embodiments, at least one RNA polynucleotide is encoded by at least one nucleic acid sequence selected from any of SEQ ID NOs: 1-31, 58, 60, 62, 64, 66, 68, 70, 72, 76, and 84-144 and homologs having at least 80% (e.g., 85%, 90%, 95%, 98%, 99%) identity with a nucleic acid sequence selected from SEQ ID NOs:1-31, 58, 60, 62, 64, 66, 68, 70, 72,76, and 84-144. In some embodiments, at least one RNA polynucleotide is encoded by at least one nucleic acid sequence selected from any of SEQ ID NOs: 1-31, 58, 60, 62, 64, 66, 68, 70, 72, 76, and 84-144 and homologs having at least 90% (90%, 91%, 92%, 93%, 94%, 95%, 96%, 97%, 98%, 99%, 99.8% or 99.9%) identity with a nucleic acid sequence selected from SEQ ID NO:1-31, 58, 60, 62, 64, 66, 68, 70, 72, 76, and 84-144. In some embodiments, at least one RNA polynucleotide is encoded by at least one fragment of a nucleic acid sequence selected from any of SEQ ID NOs: 1-31, 58, 60, 62, 64, 66, 68, 70, 72, 76, and 84-144 and homologs having at least 80% (e.g., 85%, 90%, 95%, 98%, 99%) identity with a nucleic acid sequence selected from SEQ ID NO:1-31, 58, 60, 62, 64, 66, 68, 70, 72, 76, and 84-144.

In some embodiments, at least one RNA polynucleotide is encoded by at least one nucleic acid sequence selected from any of nucleic acids disclosed herein, or homologs having at least 80% (e.g., 85%, 90%, 95%, 98%, 99%) identity with a nucleic acid sequence disclosed herein.

In any of the above-described embodiments in the preceding paragraphs, the HCMV RNA polynucleotides may further comprise additional sequences, for example, one or more linker sequences or one or more sequence tags, such as FLAG-tag and histidine tag.

In some embodiments, at least one RNA polynucleotide encodes an antigenic polypeptide having at least 90% identity to the amino acid sequence of any of SEQ ID NOs: 32-52. In some embodiments, at least one RNA polynucleotide encodes an antigenic polypeptide having at least 95% identity to the amino acid sequence of any of SEQ ID NOs: 32-52. In some embodiments, at least one RNA polynucleotide encodes an antigenic polypeptide having at least 96% identity to the amino acid sequence of any of SEQ ID NOs:32-52. In some embodiments, at least one RNA polynucleotide encodes an antigenic polypeptide having at least 97% identity to the amino acid sequence of any of SEQ ID NOs: 32-52. In some embodiments, at least one RNA polynucleotide encodes an antigenic polypeptide having at least 98% identity to the amino acid sequence of SEQ ID NOs: 32-52. In some embodiments, at least one RNA polynucleotide encodes an antigenic polypeptide having at least 99% identity to the amino acid sequence of SEQ ID Nos: 32-52.

In some embodiments, the open reading from which the HCMV polypeptide is encoded is codon-optimized. In some embodiments, the at least one RNA polynucleotide encodes an antigenic protein of SEQ ID NO: 32, and wherein the RNA polynucleotide is codon optimized mRNA. In some embodiments, the at least one RNA polynucleotide encodes an antigenic protein of SEQ ID NO: 33, and wherein the RNA polynucleotide is codon optimized mRNA. In some embodiments, the at least one RNA polynucleotide encodes an antigenic protein of SEQ ID NO: 34, and wherein the RNA polynucleotide is codon optimized mRNA. In some embodiments, the at least one RNA polynucleotide encodes an antigenic protein of SEQ ID NO: 38, and wherein the RNA polynucleotide is codon optimized mRNA. In some embodiments, the at least one RNA polynucleotide encodes an antigenic protein of SEQ ID NO: 40, and wherein the RNA polynucleotide is codon optimized mRNA. In some embodiments, the at least one RNA polynucleotide encodes an antigenic protein of SEQ ID NO: 42, and wherein the RNA polynucleotide is codon optimized mRNA. In some embodiments, the at least one RNA polynucleotide encodes an antigenic protein of SEQ ID NO: 47, and wherein the RNA polynucleotide is codon optimized mRNA. In some embodiments, the at least one RNA polynucleotide encodes an antigenic protein of SEQ ID NO: 50, and wherein the RNA polynucleotide is codon optimized mRNA.

In some embodiments, the at least one RNA polynucleotide encodes an antigenic protein of SEQ ID NO: 32, and wherein the RNA polynucleotide has less than 80% identity to wild-type mRNA sequence. In some embodiments, the at least one RNA polynucleotide encodes an antigenic protein of SEQ ID NO: 32, and wherein the RNA polynucleotide has greater than 80% identity to wild-type mRNA sequence, but does not include wild-type mRNA sequence.

In some embodiments, the at least one RNA polynucleotide encodes an antigenic protein of SEQ ID NO: 33, and wherein the RNA polynucleotide has less than 80% identity to wild-type mRNA sequence. In some embodiments, the at least one RNA polynucleotide encodes an antigenic protein of SEQ ID NO: 33, and wherein the RNA polynucleotide has greater than 80% identity to wild-type mRNA sequence, but does not include wild-type mRNA sequence.

In some embodiments, the at least one RNA polynucleotide encodes an antigenic protein of SEQ ID NO: 34, and wherein the RNA polynucleotide has less than 80% identity to wild-type mRNA sequence. In some embodiments, the at least one RNA polynucleotide encodes an antigenic protein of SEQ ID NO: 34, and wherein the RNA polynucleotide has greater than 80% identity to wild-type mRNA sequence, but does not include wild-type mRNA sequence.

In some embodiments, the at least one RNA polynucleotide encodes an antigenic protein of SEQ ID NO: 38, and wherein the RNA polynucleotide has less than 80% identity to wild-type mRNA sequence. In some embodiments, the at least one RNA polynucleotide encodes an antigenic protein of SEQ ID NO: 38, and wherein the RNA polynucleotide has greater than 80% identity to wild-type mRNA sequence, but does not include wild-type mRNA sequence.

In some embodiments, the at least one RNA polynucleotide encodes an antigenic protein of SEQ ID NO: 40, and wherein the RNA polynucleotide has less than 80% identity to wild-type mRNA sequence. In some embodiments, the at least one RNA polynucleotide encodes an antigenic protein of SEQ ID NO: 40, and wherein the RNA polynucleotide has greater than 80% identity to wild-type mRNA sequence, but does not include wild-type mRNA sequence.

In some embodiments, the at least one RNA polynucleotide encodes an antigenic protein of SEQ ID NO: 42, and wherein the RNA polynucleotide has less than 80% identity to wild-type mRNA sequence. In some embodiments, the at least one RNA polynucleotide encodes an antigenic protein of SEQ ID NO: 42, and wherein the RNA polynucleotide has greater than 80% identity to wild-type mRNA sequence, but does not include wild-type mRNA sequence.

In some embodiments, the at least one RNA polynucleotide is encoded by a sequence selected from SEQ ID NO: 1-31 and 84-144 and includes at least one chemical modification.

In some embodiments, the HCMV vaccine is multivalent. In some embodiments, the RNA polynucleotide comprises a polynucleotide sequence derived from a virus strain or isolate selected from VR1814 VR6952, VR3480B1 (ganciclovir resistant), VR4760 (ganciclovir and foscarnet resistant), Towne, TB40/E, AD169, Merlin, and Toledo.

Some embodiments of the present disclosure provide a HCMV vaccine that includes at least one ribonucleic acid (RNA) polynucleotide having an open reading frame encoding at least one HCMV antigenic polypeptide or an immunogenic fragment thereof and at least one 5′ terminal cap. In some embodiments, a 5′ terminal cap is 7mG(5′)ppp(5′)NlmpNp.

Some embodiments of the present disclosure provide a HCMV vaccine that includes at least one ribonucleic acid (RNA) polynucleotide having an open reading frame encoding at least one HCMV antigenic polypeptide or an immunogenic fragment thereof, wherein the at least one ribonucleic acid (RNA) polynucleotide has at least one chemical modification. In some embodiments, the at least one ribonucleic acid (RNA) polynucleotide further comprises a second chemical modification. In some embodiments, the at least one ribonucleic acid (RNA) polynucleotide having at least one chemical modification has a 5′ terminal cap. In some embodiments, the at least one chemical modification is selected from pseudouridine, N1-methylpseudouridine, N1-ethylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Some embodiments of the present disclosure provide a HCMV vaccine that includes at least one ribonucleic acid (RNA) polynucleotide having an open reading frame encoding at least one HCMV antigenic polypeptide or an immunogenic fragment thereof, wherein at least 80% (e.g., 85%, 90%, 95%, 98%, 99%, 100%) of the uracil in the open reading frame have a chemical modification, optionally wherein the vaccine is formulated in a lipid nanoparticle. In some embodiments, 100% of the uracil in the open reading frame have a chemical modification. In some embodiments, a chemical modification is in the 5-position of the uracil. In some embodiments, a chemical modification is a N1-methyl pseudouridine.

Some embodiments of the present disclosure provide a HCMV vaccine that is formulated within a cationic lipid nanoparticle, also referred to herein as ionizable cationic lipid nanoparticles, ionizable lipid nanoparticles and lipid nanoparticles, which are used interchangeably. In some embodiments, the lipid nanoparticle comprises a cationic lipid, a PEG-modified lipid, a sterol and a non-cationic lipid. In some embodiments, the cationic lipid is an ionizable cationic lipid and the non-cationic lipid is a neutral lipid, and the sterol is a cholesterol. In some embodiments, the cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some embodiments, the lipid nanoparticle has a molar ratio of about 20-60% cationic lipid, about 5-25% non-cationic lipid, about 25-55% sterol, and about 0.5-15% PEG-modified lipid. In some embodiments, the nanoparticle has a polydiversity value of less than 0.4. In some embodiments, the nanoparticle has a net neutral charge at a neutral pH. In some embodiments, the nanoparticle has a mean diameter of 50-200 nm.

Some embodiments of the present disclosure provide methods of inducing an antigen specific immune response in a subject, comprising administering to the subject a HCMV RNA vaccine in an amount effective to produce an antigen specific immune response. In some embodiments, an antigen specific immune response comprises a T cell response or a B cell response. In some embodiments, an antigen specific immune response comprises a T cell response and a B cell response. In some embodiments, a method of producing an antigen specific immune response involves a single administration of the vaccine. In some embodiments, a method further includes administering to the subject a booster dose of the vaccine. In some embodiments, a vaccine is administered to the subject by intradermal or intramuscular injection.

Also provided herein are HCMV RNA vaccines for use in a method of inducing an antigen specific immune response in a subject, the method comprising administering the vaccine to the subject in an amount effective to produce an antigen specific immune response.

Further provided herein are uses of HCMV RNA vaccines in the manufacture of a medicament for use in a method of inducing an antigen specific immune response in a subject, the method comprising administering the vaccine to the subject in an amount effective to produce an antigen specific immune response.

Further provided herein are methods of preventing or treating HCMV infection comprising administering to a subject the vaccine of the present disclosure.

The HCMV vaccine disclosed herein may be formulated in an effective amount to produce an antigen specific immune response in a subject.

In some embodiments, an anti-HCMV antigenic polypeptide antibody titer produced in the subject is increased by at least 1 log relative to a control. In some embodiments, the anti-HCMV antigenic polypeptide antibody titer produced in the subject is increased by 1-3 log relative to a control. In some embodiments, an anti-HCMV antigenic polypeptide antibody titer produced in the subject is increased at least 2 times relative to a control. In some embodiments, the anti-HCMV antigenic polypeptide antibody titer produced in the subject is increased at least 5 times relative to a control. In some embodiments, the anti-HCMV antigenic polypeptide antibody titer produced in the subject is increased at least 10 times relative to a control. In some embodiments, the anti-HCMV antigenic polypeptide antibody titer produced in the subject is increased 2-10 times relative to a control.

In some embodiments, the control is an anti-HCMV antigenic polypeptide antibody titer produced in a subject who has not been administered HCMV vaccine.

In some embodiments, the control is an anti-HCMV antigenic polypeptide antibody titer produced in a subject who has been administered a live attenuated or inactivated HCMV vaccine.

In some embodiments, the control is an anti-HCMV antigenic polypeptide antibody titer produced in a subject who has been administered a recombinant or purified HCMV protein vaccine.

In some embodiments, the effective amount is a dose equivalent to an at least 2-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 4-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 10-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 100-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to a 2-1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a total dose of 50-1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

Other aspects of the present disclosure provide methods of inducing an antigen specific immune response in a subject, including administering to a subject the HCMV vaccine disclosed herein in an effective amount to produce an antigen specific immune response in a subject.

In some embodiments, an anti-HCMV antigenic polypeptide antibody titer produced in the subject is increased by at least 1 log relative to a control. In some embodiments, an anti-HCMV antigenic polypeptide antibody titer produced in the subject is increased by 1-3 log relative to a control. In some embodiments, an anti-HCMV antigenic polypeptide antibody titer produced in the subject is increased at least 2 times relative to a control. In some embodiments, the anti-HCMV antigenic polypeptide antibody titer produced in the subject is increased at least 5 times relative to a control. In some embodiments, the anti-HCMV antigenic polypeptide antibody titer produced in the subject is increased at least 10 times relative to a control. In some embodiments, the anti-HCMV antigenic polypeptide antibody titer produced in the subject is increased 2-10 times relative to a control.

In some embodiments, the control is an anti-HCMV antigenic polypeptide antibody titer produced in a subject who has not been administered HCMV vaccine.

In some embodiments, the control is an anti-HCMV antigenic polypeptide antibody titer produced in a subject who has been administered a live attenuated or inactivated HCMV vaccine.

In some embodiments, the control is an anti-HCMV antigenic polypeptide antibody titer produced in a subject who has been administered a recombinant or purified HCMV protein vaccine.

In some embodiments, the effective amount is a dose equivalent to an at least 2-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant HCMV protein vaccine or a live attenuated HCMV vaccine.

In some embodiments, the effective amount is a dose equivalent to an at least 4-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 10-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 100-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to a 2-1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a total dose of 50-1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

Other aspects of the present disclosure provide HCMV vaccines containing a signal peptide linked to a HCMV antigenic polypeptide.

In some embodiments, the HCMV antigenic polypeptide is a HCMV glycoprotein or an antigenic fragment thereof. In some embodiments, the HCMV antigenic polypeptide is a HCMV gB, gM, gN, gH, gL, gO, UL 83, UL123, UL128, UL130, or UL131A protein or an antigenic fragment or epitope thereof. In some embodiments, the HCMV glycoprotein is selected from HCMV gB, gM, gN, gH, gL, and gO.

In some embodiments, the HCMV glycoprotein is HCMV gH. In some embodiments, the HCMV glycoprotein is HCMV gL. In some embodiments, the HCMV glycoprotein is HCMV gB. In some embodiments, the HCMV protein is HCMV UL128. In some embodiments, the HCMV protein is HCMV UL130. In some embodiments, the HCMV protein is HCMV UL131A. In some embodiments, the HCMV protein is HCMV UL83. In some embodiments, the HCMV protein is HCMV UL123. In some embodiments, the HCMV glycoprotein is a variant HCMV gH polypeptide. In some embodiments, the HCMV glycoprotein is a variant HCMV gL polypeptide. In some embodiments, the HCMV glycoprotein is a variant HCMV gB polypeptide.

In some embodiments, the signal peptide is an IgE signal peptide. In some embodiments, the signal peptide is an IgE HC (Ig heavy chain epsilon-1) signal peptide. In some embodiments, the signal peptide has the amino acid sequence MDWTWILFLVAAATRVHS (SEQ ID NO: 53).

In some embodiments, the signal peptide is an IgGκ signal peptide. In some embodiments, the signal peptide has the amino acid sequence METPAQLLFLLLLWLPDTTG (SEQ ID NO: 54).

In some embodiments, the HCMV vaccine comprises at least one RNA polynucleotide encoding gH, gL, UL128, UL130, and UL131A, or antigenic fragments or epitopes thereof, and at least one RNA polynucleotide encoding gB, or an antigenic fragment or epitope thereof.

Further provided herein are uses of HCMV vaccines for prevention of congenital HCMV infection. Further provided herein are methods of administering HCMV vaccines to a women of child-bearing age.

Aspects of the invention relate to a human cytomegalovirus (HCMV) vaccine comprising: i) at least one RNA polynucleotide having one or more open reading frames encoding HCMV antigenic polypeptides gH, gL, UL128, UL130, and/or UL131A, or antigenic fragments or epitopes thereof; ii) an RNA polynucleotide having an open reading frame encoding HCMV antigenic polypeptide gB, or an antigenic fragment or epitope thereof; iii) an RNA polynucleotide having an open reading frame encoding HCMV antigenic polypeptide pp65, or an antigenic fragment or epitope thereof, and iv) a pharmaceutically acceptable carrier or excipient.

In some embodiments, the vaccine comprises: an RNA polynucleotide having an open reading frame encoding HCMV antigenic polypeptide gH, or an antigenic fragment or epitope thereof; an RNA polynucleotide having an open reading frame encoding HCMV antigenic polypeptide gL, or an antigenic fragment or epitope thereof; an RNA polynucleotide having an open reading frame encoding HCMV antigenic polypeptide UL128, or an antigenic fragment or epitope thereof; an RNA polynucleotide having an open reading frame encoding HCMV antigenic polypeptide UL130, or an antigenic fragment or epitope thereof; and an RNA polynucleotide having an open reading frame encoding HCMV antigenic polypeptide UL131A. or an antigenic fragment or epitope thereof.

In some embodiments, at least one RNA polynucleotide has an open reading frame encoding two or more HCMV antigenic polypeptides. In some embodiments, one or more of the open reading frames is codon-optimized. In some embodiments, the pp65 polypeptide contains a deletion of amino acids 435-438. In some embodiments, at least one RNA polynucleotide is encoded by at least one nucleic acid sequence selected from SEQ ID NOs: 58, 60, 62, 64, 66, 68, 70, and 84-144. In some embodiments, at least one of the RNA polynucleotides encodes an antigenic polypeptide having at least 90% identity to any of the amino acid sequences of SEQ ID NO: 59, SEQ ID NO: 61, SEQ ID NO: 63, SEQ ID NO: 65, SEQ ID NO: 67, SEQ ID NO: 69, SEQ ID NO: 71, and 80-83.

In some embodiments, at least one of the RNA polynucleotides encodes an antigenic polypeptide having at least 95% identity to any of the amino acid sequences of SEQ ID NO: 59, SEQ ID NO: 61, SEQ ID NO: 63, SEQ ID NO: 65, SEQ ID NO: 67, SEQ ID NO: 69, SEQ ID NO: 71, and 80-83. In some embodiments, at least one of the RNA polynucleotides encodes an antigenic polypeptide having at least 96% identity to any of the amino acid sequences of SEQ ID NO: 59, SEQ ID NO: 61, SEQ ID NO: 63, SEQ ID NO: 65, SEQ ID NO: 67, SEQ ID NO: 69, SEQ ID NO: 71, and 80-83. In some embodiments, at least 97% identity to any of the amino acid sequences of SEQ ID NO: 59, SEQ ID NO: 61, SEQ ID NO: 63, SEQ ID NO: 65, SEQ ID NO: 67, SEQ ID NO: 69, SEQ ID NO: 71, and 80-83. In some embodiments, at least 98% identity to any of the amino acid sequences SEQ ID NO: 59, SEQ ID NO: 61, SEQ ID NO: 63, SEQ ID NO: 65, SEQ ID NO: 67, SEQ ID NO: 69, SEQ ID NO: 71, and 80-83. In some embodiments, at least 99% identity to any of the amino acid sequences of SEQ ID NO: 59, SEQ ID NO: 61, SEQ ID NO: 63, SEQ ID NO: 65, SEQ ID NO: 67, SEQ ID NO: 69, SEQ ID NO: 71, and 80-83.

In some embodiments, at least one RNA polynucleotide encodes an antigenic protein of SEQ ID NOs: 59, 61, 63, 65, or 67 and the RNA polynucleotide has less than 80% identity to wild-type mRNA sequence or has greater than 80% identity to wild-type mRNA sequence, but does not include wild-type mRNA sequence. In some embodiments, at least one RNA polynucleotide encodes an antigenic protein of SEQ ID NO.: 69, and wherein the RNA polynucleotide has less than 80% identity to wild-type mRNA sequence or has greater than 80% identity to wild-type mRNA sequence, but does not include wild-type mRNA sequence. In some embodiments, at least one RNA polynucleotide encodes an antigenic protein of SEQ ID NO.: 71, and wherein the RNA polynucleotide has less than 80% identity to wild-type mRNA sequence or has greater than 80% identity to wild-type mRNA sequence, but does not include wild-type mRNA sequence.

In some embodiments, at least one RNA polynucleotide includes at least one chemical modification. In some embodiments, the vaccine is multivalent. In some embodiments, the RNA polynucleotide comprises a polynucleotide sequence derived from a virus strain or isolate selected from VR 1814, VR6952, VR3480B1. VR4760, Towne, TB40/E, AD169, Merlin, and Toledo. In some embodiments, the HCMV vaccine further comprises a second chemical modification.

In some embodiments, the chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments, 80% of the uracil in the open reading frame have a chemical modification. In some embodiments, 100% of the uracil in the open reading frame have a chemical modification. In some embodiments, the chemical modification is in the 5-position of the uracil. In some embodiments, the chemical modification is N1-methylpseudouridine, N1-ethylpseudouridine. In some embodiments, the vaccine is formulated within a lipid nanoparticle. In some embodiments, the lipid nanoparticle comprises a cationic lipid, a PEG-modified lipid, a sterol and a non-cationic lipid. In some embodiments, the cationic lipid is an ionizable cationic lipid and the non-cationic lipid is a neutral lipid, and the sterol is a cholesterol. In some embodiments, the cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In some embodiments, the lipid nanoparticle has a molar ratio of about 20-60% cationic lipid, about 5-25% non-cationic lipid, about 25-55% sterol, and about 0.5-15% PEG-modified lipid. In some embodiments, the nanoparticle has a polydiversity value of less than 0.4. In some embodiments, the nanoparticle has a net neutral charge at a neutral pH. In some embodiments, the nanoparticle has a mean diameter of 50-200 nm.

Aspects of the invention relate to methods of inducing an antigen specific immune response in a subject, comprising administering any of the vaccines described herein to the subject in an effective amount to produce an antigen specific immune response. In some embodiments of methods described herein, the antigen specific immune response comprises a T cell response. In some embodiments of methods described herein, the antigen specific immune response comprises a B cell response. In some embodiments of methods described herein, the antigen specific immune response comprises a T cell response and a B cell response.

In some embodiments of methods described herein, the method of producing an antigen specific immune response involves a single administration of the vaccine. In some embodiments of methods described herein, methods further comprise administering a booster dose of the vaccine. In some embodiments of methods described herein, the vaccine is administered to the subject by intradermal or intramuscular injection.

Aspects of the invention relate to HCMV vaccines for use in a method of inducing an antigen specific immune response in a subject, the method comprising administering the vaccine to the subject in an effective amount to produce an antigen specific immune response.

Aspects of the invention relate to use of an HCMV vaccine described herein in the manufacture of a medicament for use in a method of inducing an antigen specific immune response in a subject, the method comprising administering the vaccine to the subject in an effective amount to produce an antigen specific immune response.

Aspects of the invention relate to methods of preventing or treating HCMV infection comprising administering to a subject any of the vaccines described herein.

Aspects of the invention relate to HCMV vaccines formulated in an effective amount to produce an antigen specific immune response in a subject. In some embodiments, an anti-HCMV antigenic polypeptide antibody titer produced in the subject is increased by at least 1 log relative to a control, or 1-3 log relative to a control. In some embodiments, an anti-HCMV antigenic polypeptide antibody titer produced in the subject is increased at least 2 times relative to a control, at least 5 times relative to a control, at least 10 times relative to a control, or 2-10 times relative to a control.

In some embodiments, the control is an anti-HCMV antigenic polypeptide antibody titer produced in a subject who has not been administered HCMV vaccine. In some embodiments, the control is an anti-HCMV antigenic polypeptide antibody titer produced in a subject who has been administered a live attenuated or inactivated HCMV vaccine. In some embodiments, the control is an anti-HCMV antigenic polypeptide antibody titer produced in a subject who has been administered a recombinant or purified HCMV protein vaccine.

In some embodiments, the effective amount is a dose equivalent to an at least 2-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 4-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 10-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 100-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to an at least 1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a dose equivalent to a 2-1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount is a total dose of 50-1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

In some embodiments of methods disclosed herein, an anti-HCMV antigenic polypeptide antibody titer produced in the subject is increased by at least 1 log relative to a control or by 1-3 log relative to a control. In some embodiments of methods disclosed herein, an anti-HCMV antigenic polypeptide antibody titer produced in the subject is increased at least 2 times relative to a control, at least 5 times relative to a control, at least 10 times relative to a control, or 2-10 times relative to a control.

In some embodiments of methods disclosed herein, the control is an anti-HCMV antigenic polypeptide antibody titer produced in a subject who has not been administered HCMV vaccine. In some embodiments of methods disclosed herein, the control is an anti-HCMV antigenic polypeptide antibody titer produced in a subject who has been administered a live attenuated or inactivated HCMV vaccine. In some embodiments of methods disclosed herein, the control is an anti-HCMV antigenic polypeptide antibody titer produced in a subject who has been administered a recombinant or purified HCMV protein vaccine.

In some embodiments of methods disclosed herein, the effective amount is a dose equivalent to an at least 2-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant HCMV protein vaccine or a live attenuated HCMV vaccine.

In some embodiments of methods disclosed herein, the effective amount is a dose equivalent to an at least 4-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments of methods disclosed herein, the effective amount is a dose equivalent to an at least 10-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments of methods disclosed herein, the effective amount is a dose equivalent to an at least 100-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments of methods disclosed herein, the effective amount is a dose equivalent to an at least 1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments of methods disclosed herein, the effective amount is a dose equivalent to a 2-1000-fold reduction in the standard of care dose of a recombinant HCMV protein vaccine, and wherein an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments of methods disclosed herein, the effective amount is a total dose of 50-1000 μg. In some embodiments of methods disclosed herein, the effective amount is a total dose of 100 μg. In some embodiments of methods disclosed herein, the effective amount is a dose of 25 μg administered to the subject a total of two times. In some embodiments of methods disclosed herein, the effective amount is a dose of 100 μg administered to the subject a total of two times. In some embodiments of methods disclosed herein, the effective amount is a dose of 400 μg administered to the subject a total of two times. In some embodiments of methods disclosed herein, the effective amount is a dose of 500 μg administered to the subject a total of two times.

Aspects of the invention relate to an HCMV vaccine, comprising: i) HCMV antigenic polypeptides gH, gL, UL128, UL130, and/or UL131A, or antigenic fragments or epitopes thereof; ii) HCMV antigenic polypeptide gB, or an antigenic fragment or epitope thereof; and iii) HCMV antigenic polypeptide pp65, or an antigenic fragment or epitope thereof.

In some embodiments, one or more of the HCMV antigenic polypeptides comprises a signal sequence linked to the HCMV antigenic polypepide, optionally wherein the signal peptide is an IgE signal peptide. In some embodiments, the signal peptide is an IgE HC (Ig heavy chain epsilon-1) signal peptide. In some embodiments, the signal peptide has the amino acid sequence MDWTWILFLVAAATRVHS (SEQ ID NO: 53). In some embodiments, the signal peptide is an IgGκ signal peptide. In some embodiments, the signal peptide has the amino acid sequence METPAQLLFLLLLWLPDTTG (SEQ ID NO: 54). In some embodiments, the pp65 polypeptide contains a deletion of amino acids 435-438.

In some embodiments of methods disclosed herein, the subject is an immunocompromised organ transplant recipient. In some embodiments of methods disclosed herein, the transplant recipient is a hematopoietic cell transplant recipient or a solid organ transplant recipient.

Aspects of the invention relate to methods of treating an immunocompromised organ transplant recipient subject having a cytomegalovirus (CMV) infection, comprising administering to the subject a therapeutically effective amount of a human cytomegalovirus (HCMV) vaccine comprising: i) at least one RNA polynucleotide having one or more open reading frames encoding HCMV antigenic polypeptides gH, gL, UL128, UL130, and/or UL131A, or antigenic fragments or epitopes thereof; ii) an RNA polynucleotide having an open reading frame encoding HCMV antigenic polypeptide gB, or an antigenic fragment or epitope thereof; iii) an RNA polynucleotide having an open reading frame encoding HCMV antigenic polypeptide pp65, or an antigenic fragment or epitope thereof; and iv) a pharmaceutically acceptable carrier or excipient.

In some embodiments of methods disclosed herein, the vaccine comprises: an RNA polynucleotide having an open reading frame encoding HCMV antigenic polypeptide gH, or an antigenic fragment or epitope thereof; an RNA polynucleotide having an open reading frame encoding HCMV antigenic polypeptide gL, or an antigenic fragment or epitope thereof; an RNA polynucleotide having an open reading frame encoding HCMV antigenic polypeptide UL128, or an antigenic fragment or epitope thereof; an RNA polynucleotide having an open reading frame encoding HCMV antigenic polypeptide UL130, or an antigenic fragment or epitope thereof; and an RNA polynucleotide having an open reading frame encoding HCMV antigenic polypeptide UL131A, or an antigenic fragment or epitope thereof.

In some embodiments of methods disclosed herein, at least one RNA polynucleotide has an open reading frame encoding two or more HCMV antigenic polypeptides. In some embodiments of methods disclosed herein, one or more of the open reading frames is codon-optimized. In some embodiments of methods disclosed herein, the pp65 polypeptide contains a deletion of amino acids 435-438.

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first antigenic polypeptide or a concatemeric polypeptide, wherein the RNA polynucleotide does not include a stabilization element, and wherein an adjuvant is not coformulated or co-administered with the vaccine.

In other aspects the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is 1-5 ug, 5-10 ug, 10-15 ug, 15-20 ug, 10-25 ug, 20-25 ug, 20-50 ug, 30-50 ug, 40-50 ug, 40-60 ug, 60-80 ug, 60-100 ug, 50-100 ug, 80-120 ug, 40-120 ug, 40-150 ug, 50-150 ug, 50-200 ug, 80-200 ug, 100-200 ug, 120-250 ug, 150-250 ug, 180-280 ug, 200-300 ug, 50-300 ug, 80-300 ug, 100-300 ug, 40-300 ug, 50-350 ug, 100-350 ug, 200-350 ug, 300-350 ug, 320-400 ug, 40-380 ug, 40-100 ug, 100-400 ug, 200-400 ug, or 300-400 ug per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100 fold higher level in the local lymph node in comparison with the distal lymph node. In other embodiments the nucleic acid vaccine is chemically modified and in other embodiments the nucleic acid vaccine is not chemically modified.

Aspects of the invention provide a nucleic acid vaccine comprising one or more RNA polynucleotides having an open reading frame encoding a first antigenic polypeptide or a concatemeric polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typially expressed as the highest serum dilution required to achieve a 50% reduction in the number of plaques.

In preferred aspects, vaccines of the invention (e.g., LNP-encapsulated mRNA vaccines) produce prophylactically- and/or therapeutically- efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In exemplary aspects of the invention, antigen-specific antibodies are measured in units of μg/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

Also provided are nucleic acid vaccines comprising one or more RNA polynucleotides having an open reading frame encoding a first antigenic polypeptide or a concatemeric polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 ug and 400 μg of one or more RNA polynucleotides having an open reading frame comprising at least one chemical modification or optionally no nucleotide modification, the open reading frame encoding a first antigenic polypeptide or a concatemeric polypeptide, and a pharmaceutically acceptable carrier or excipient, formulated for delivery to a human subject. In some embodiments, the vaccine further comprises a lipid nanoparticle.

Aspects of the invention provide methods of creating, maintaining or restoring antigenic memory to a virus in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 ug/kg and 400 ug/kg of a nucleic acid vaccine comprising one or more RNA polynucleotides having an open reading frame encoding a first antigenic polypeptide or a concatemeric polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Other aspects provide nucleic acid vaccines comprising an LNP formulated RNA polynucleotide having an open reading frame comprising no nucleotide modifications (unmodified), the open reading frame encoding a first antigenic polypeptide or a concatemeric polypeptide, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

The data presented in the Examples demonstrate significant enhanced immune responses using the formulations of the invention. Surprisingly, in contrast to prior art reports that it was preferable to use chemically unmodified mRNA formulated in a carrier for the production of vaccines, it is described herein that chemically modified mRNA-LNP vaccines required a much lower effective mRNA dose than unmodified mRNA, i.e., tenfold less than unmodified mRNA when formulated in carriers other than LNP. Both the chemically modified and unmodified RNA vaccines of the invention produce better immune responses than mRNA vaccines formulated in a different lipid carrier.

In other aspects the invention encompasses a method of treating an elderly subject age 60 years or older comprising administering to the subject a nucleic acid vaccine comprising one or more RNA polynucleotides having an open reading frame encoding an antigenic polypeptide or a concatemeric polypeptide in an effective amount to vaccinate the subject.

In other aspects the invention encompasses a method of treating a young subject age 17 years or younger comprising administering to the subject a nucleic acid vaccine comprising one or more RNA polynucleotides having an open reading frame encoding an antigenic polypeptide or a concatemeric polypeptide in an effective amount to vaccinate the subject.

In other aspects the invention encompasses a method of treating an adult subject comprising administering to the subject a nucleic acid vaccine comprising one or more RNA polynucleotides having an open reading frame encoding an antigenic polypeptide or a concatemeric polypeptide in an effective amount to vaccinate the subject.

In some aspects the invention is a method of vaccinating a subject with a combination vaccine including at least two nucleic acid sequences encoding antigens wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject. In some embodiments, the sub therapeutic dosage of each individual nucleic acid encoding an antigen is 1, 2, 3, 4, 5, 6, 7, 8, 9, 10, 11, 12, 13, 14, 15, 16, 17, 18, 19, or 20 micrograms. In other embodiments the nucleic acid vaccine is chemically modified and in other embodiments the nucleic acid vaccine is not chemically modified.

The RNA polynucleotide is one of SEQ ID NO: 58, 60, 62, 64, 66, 68, 70, and 84-144 and includes at least one chemical modification. In other embodiments the RNA polynucleotide is one of SEQ ID NO: 1 58, 60, 62, 64, 66, 68, 70, 84-144 and does not include any nucleotide modifications, or is unmodified.

Further aspects of the invention relate to methods of preventing or treating HCMV infection comprising administering to a subject a therapeutically effective amount of: (i) a first HCMV vaccine comprising an RNA polynucleotide having an open reading frame encoding HCMV antigenic polypeptide pp65, or an antigenic fragment or epitope thereof, and (ii) a second HCMV vaccine comprising at least one RNA polynucleotide having one or more open reading frames encoding HCMV antigenic polypeptides gH, gL, UL128, UL130, and/or UL131A, or antigenic fragments or epitopes thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof.

Further aspects of the invention relate to methods of treating an immunocompromised organ transplant recipient subject having a cytomegalovirus (CMV) infection, comprising administering to the subject a therapeutically effective amount of: (i) a first HCMV vaccine comprising an RNA polynucleotide having an open reading frame encoding HCMV antigenic polypeptide pp65, or an antigenic fragment or epitope thereof, and (ii) a second HCMV vaccine comprising at least one RNA polynucleotide having one or more open reading frames encoding HCMV antigenic polypeptides gH, gL, UL128, UL130, and/or UL131A. or antigenic fragments or epitopes thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof.

In some embodiments of methods described herein, the first HCMV vaccine is administered at least 1 week, at least 2 weeks, or at least 3 weeks prior to administering the second HCMV vaccine. In some embodiments of methods described herein, the pp65 polypeptide contains a deletion of amino acids 435-438.

In some embodiments of methods described herein, the second HCMV vaccine comprises: an RNA polynucleotide having an open reading frame encoding HCMV antigenic polypeptide gH, or an antigenic fragment or epitope thereof; an RNA polynucleotide having an open reading frame encoding HCMV antigenic polypeptide gL, or an antigenic fragment or epitope thereof; an RNA polynucleotide having an open reading frame encoding HCMV antigenic polypeptide UL128, or an antigenic fragment or epitope thereof; an RNA polynucleotide having an open reading frame encoding HCMV antigenic polypeptide UL130, or an antigenic fragment or epitope thereof; an RNA polynucleotide having an open reading frame encoding HCMV antigenic polypeptide UL131A, or an antigenic fragment or epitope thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof.

In some embodiments of methods described herein, one or more of the RNA polynucleotides in the first and/or second HCMV vaccines are codon optimized. In some embodiments of methods described herein, at least one of the RNA polynucleotides encodes an antigenic polypeptide having at least 90% identity to any of the amino acid sequences of SEQ ID NO: 59, SEQ ID NO: 61, SEQ ID NO: 63, SEQ ID NO: 65, SEQ ID NO: 67, SEQ ID NO: 69, SEQ ID NO: 71. SEQ ID NO: 73, SEQ ID NO: 77, and SEQ ID NOs: 80-83.

In some embodiments of methods described herein, one or more of the RNA polynucleotides includes at least one chemical modification. In some embodiments of methods described herein, at least one chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments of methods described herein, the first and/or second HCMV vaccine is formulated within a lipid nanoparticle. In some embodiments of methods described herein, the lipid nanoparticle(s) comprise a molar ratio of 20-60% ionizable cationic lipid, 5-25% non-cationic lipid, 25-55% sterol, and 0.5-15% PEG-modified lipid. In some embodiments of methods described herein, the cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In some embodiments of methods described herein, the first and/or second HCMV vaccine further comprises a pharmaceutically acceptable carrier or excipient. In some embodiments of methods described herein, the transplant recipient is a hematopoietic cell transplant recipient or a solid organ transplant recipient. In some embodiments of methods described herein, at least one RNA polynucleotide further encodes at least one 5′ terminal cap, 7mG(5′)ppp(5′)NlmpNp.

Further aspects of the invention relate to a kit comprising: (i) a first HCMV vaccine comprising an RNA polynucleotide having an open reading frame encoding HCMV antigenic polypeptide pp65, or an antigenic fragment or epitope thereof, and (ii) a second HCMV vaccine comprising at least one RNA polynucleotide having one or more open reading frames encoding HCMV antigenic polypeptides gH, gL, UL128, UL130, and/or UL131A, or antigenic fragments or epitopes thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof.

In some embodiments of kits described herein, the first HCMV vaccine is administered at least 1 week, at least 2 weeks, or at least 3 weeks prior to administering the second HCMV vaccine. In some embodiments of methods described herein, the pp65 polypeptide contains a deletion of amino acids 435-438.

In some embodiments of kits described herein, the second HCMV vaccine comprises: an RNA polynucleotide having an open reading frame encoding HCMV antigenic polypeptide gH, or an antigenic fragment or epitope thereof; an RNA polynucleotide having an open reading frame encoding HCMV antigenic polypeptide gL, or an antigenic fragment or epitope thereof; an RNA polynucleotide having an open reading frame encoding HCMV antigenic polypeptide UL128, or an antigenic fragment or epitope thereof; an RNA polynucleotide having an open reading frame encoding HCMV antigenic polypeptide UL130, or an antigenic fragment or epitope thereof; an RNA polynucleotide having an open reading frame encoding HCMV antigenic polypeptide UL131A, or an antigenic fragment or epitope thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof.

In some embodiments of kits described herein, one or more of the RNA polynucleotides in the first and/or second HCMV vaccines are codon optimized. In some embodiments of kits described herein, at least one of the RNA polynucleotides encodes an antigenic polypeptide having at least 90% identity to any of the amino acid sequences of SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 77, and SEQ ID NOs: 80-83.

In some embodiments of kits described herein, one or more of the RNA polynucleotides includes at least one chemical modification. In some embodiments of kits described herein, at least one chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments of kits described herein, the first and/or second HCMV vaccine is formulated within a lipid nanoparticle. In some embodiments of kits described herein, the lipid nanoparticle(s) comprise a molar ratio of 20-60% ionizable cationic lipid, 5-25% non-cationic lipid, 25-55% sterol, and 0.5-15% PEG-modified lipid. In some embodiments of kits described herein, the cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In some embodiments of kits described herein, the first and/or second HCMV vaccine further comprises a pharmaceutically acceptable carrier or excipient. In some embodiments of kits described herein, at least one RNA polynucleotide further encodes at least one 5′ terminal cap, 7mG(5′)ppp(5′)NlmpNp.

Kits described herein are for use in preventing or treating HCMV infection. In some embodiments of kits described herein, the subject is an immunocompromised organ transplant recipient. In some embodiments of kits described herein, the transplant recipient is a hematopoietic cell transplant recipient or a solid organ transplant recipient.

Further aspects of the invention relate to a human cytomegalovirus (HCMV) vaccine comprising: an RNA polynucleotide having an open reading frame encoding HCMV antigenic polypeptide pp65, or an antigenic fragment or epitope thereof, and a pharmaceutically acceptable carrier or excipient.

In some embodiments, the pp65 polypeptide contains a deletion of amino acids 435-438. In some embodiments, the RNA polynucleotide is codon optimized. In some embodiments, the RNA polynucleotide encodes an antigenic polypeptide having at least 90% identity to SEQ ID NO: 71 or SEQ ID NO: 82. In some embodiments, the RNA polynucleotide includes at least one chemical modification. In some embodiments, at least one chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments, the vaccine is formulated within a lipid nanoparticle. In some embodiments, the lipid nanoparticle(s) comprise a molar ratio of 20-60% ionizable cationic lipid, 5-25% non-cationic lipid, 25-55% sterol, and 0.5-15% PEG-modified lipid. In some embodiments, the cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some embodiments, the RNA polynucleotide further encodes at least one 5′ terminal cap, 7mG(5′)ppp(5′)NlmpNp.

In some embodiments, the vaccine is for use in preventing or treating HCMV infection in a subject. In some embodiments, the subject is an immunocompromised organ transplant recipient. In some embodiments, the transplant recipient is a hematopoietic cell transplant recipient or a solid organ transplant recipient.

Aspects of the invention relate to a human cytomegalovirus (HCMV) vaccine comprising: an mRNA comprising an open reading frame (ORF) encoding a HCMV antigenic polypeptide pp65, or an antigenic fragment or epitope thereof, formulated within a lipid nanoparticle, wherein the lipid nanoparticle comprises an ionizable lipid, a PEG-modified lipid, a sterol and a non-cationic lipid.

In some embodiments, the HCMV vaccine further comprises an mRNA comprising an ORF encoding one or more HCMV antigenic polypeptides selected from gH, gL, UL128, UL130, and UL131A, or antigenic fragments or epitopes thereof.

In some embodiments, the HCMV vaccine further comprises an mRNA comprising two ORFs encoding two HCMV antigenic polypeptides selected from gH, gL, UL128, UL130, and UL131A, or antigenic fragments or epitopes thereof.

In some embodiments, the HCMV vaccine further comprises an mRNA comprising three ORFs encoding three HCMV antigenic polypeptides selected from gH, gL, UL128, UL130, and UL131A, or antigenic fragments or epitopes thereof.

In some embodiments, the HCMV vaccine further comprises an mRNA comprising four ORFs encoding four HCMV antigenic polypeptides selected from gH, gL, UL128, UL130, and UL131A, or antigenic fragments or epitopes thereof.

In some embodiments, the HCMV vaccine further comprises an mRNA comprising ORFs encoding each of HCMV antigenic polypeptides gH, gL, UL128. UL130, and UL131A, or antigenic fragments or epitopes thereof.

In some embodiments, the HCMV vaccine further comprises one or more mRNAs, each mRNA comprising an ORF encoding an HCMV antigenic polypeptide selected from gH, gL, UL128. UL130, and UL131A, or antigenic fragments or epitopes thereof.

In some embodiments, the HCMV vaccine further comprises two mRNAs, each mRNA comprising a different ORF encoding an HCMV antigenic polypeptide selected from gH, gL, UL128, UL130, and UL131A, or antigenic fragments or epitopes thereof.

In some embodiments, the HCMV vaccine further comprises three mRNAs, each mRNA comprising a different ORF encoding an HCMV antigenic polypeptide selected from gH, gL, UL128, UL130, and UL131A, or antigenic fragments or epitopes thereof.

In some embodiments, the HCMV vaccine further comprises four mRNAs, each mRNA comprising a different ORF encoding an HCMV antigenic polypeptide selected from gH, gL, UL128, UL130, and UL131A, or antigenic fragments or epitopes thereof.

In some embodiments, the HCMV vaccine further comprises five mRNAs, each mRNA comprising a different ORF encoding an HCMV antigenic polypeptide selected from gH, gL, UL128, UL130, and UL131A, or antigenic fragments or epitopes thereof.

In some embodiments, the HCMV vaccine further comprises an mRNA comprising an ORF encoding HCMV antigenic polypeptide gB, or an antigenic fragment or epitope thereof.

In some embodiments, the lipid nanoparticle has a molar ratio of about 20-60% ionizable lipid, about 5-25% non-cationic lipid, about 25-55% sterol, and about 0.5-15% PEG-modified lipid. In some embodiments, the ionizable lipid comprises Compound 25, a salt or a stereoisomer thereof, or any combination thereof.

In some embodiments, each mRNA is formulated in a separate lipid nanoparticle. In some embodiments, the mRNA comprising the ORF encoding the HCMV antigenic polypeptide pp65 is in a separate lipid nanoparticle than the other mRNA. In some embodiments, all of the mRNA other than the mRNA comprising the ORF encoding the HCMV antigenic polypeptide pp65 are formulated in the same lipid nanoparticle.

In some embodiments, the mRNA comprises a chemical modification. In some embodiments, the chemical modification is N1-methyl pseudouridine (m1Ψ). In some embodiments, each U in the mRNA is a N1-methyl pseudouridine (m1Ψ).

In some embodiments, the ORFs encoding the HCMV antigenic polypeptides are encoded by the following nucleic acid sequences: gH: SEQ ID NO: 87, gL: SEQ ID NO: 90, UL128: SEQ ID NO: 89, UL130: SEQ ID NO: 91, UL131A: SEQ ID NO: 144, gB: SEQ ID NO: 86, and pp65: SEQ ID NO: 92.

In some embodiments, the HCMV antigenic polypeptides have the following amino acid sequences: gH: SEQ ID NO: 59, gL: SEQ ID NO: 3, UL128: SEQ ID NO: 63, UL130: SEQ ID NO: 65, UL131A: SEQ ID NO: 67, gB: SEQ ID NO: 69, and pp65: SEQ ID NO: 71.

In some embodiments, the mRNA further comprises a UTR encoded by SEQ ID NO: 146 and/or SEQ ID NO: 147.

In some embodiments, anti-HCMV antigenic polypeptide antibody titer produced in the subject is increased by 1-3 log relative to a control. In some embodiments, anti-HCMV antigenic polypeptide antibody titer produced in the subject is increased 2-10 times relative to a control. In some embodiments, the control is an anti-HCMV antigenic polypeptide antibody titer produced in a subject who has not been administered HCMV vaccine, a subject who has been administered a live attenuated or inactivated HCMV vaccine, or a subject who has been administered a recombinant or purified HCMV protein vaccine.

In some embodiments, the mRNA is present in the lipid nanoparticle in a total dose selected from 50-1000 μg, 35-100 μg, or 25-50 μg.

In some embodiments, the ORFs encoding the HCMV antigenic polypeptides are encoded by the following nucleic acid sequences: gH: a sequence comprising at least 90%, 95% or 98% identity to SEQ ID NO: 87; gL: a sequence comprising at least 90%, 95% or 98% identity to SEQ ID NO: 90; UL128: a sequence comprising at least 90%, 95% or 98% identity to SEQ ID NO: 89; UL130: a sequence comprising at least 90%, 95% or 98% identity to SEQ ID NO: 91: UL131A: a sequence comprising at least 90%, 95% or 98% identity to SEQ ID NO: 144; gB: a sequence comprising at least 90%, 95% or 98% identity to SEQ ID NO: 86; and pp65: a sequence comprising at least 90%, 95% or 98% identity to SEQ ID NO: 92.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C depict different protein complexes formed by hCMV proteins. The tropism of hCMV is dictated by distinct protein complexes. FIG. 1A shows the gH/gL/gB complex that mediates the entry of hCMV into fibroblasts. FIG. 1B shows the pentameric complex containing gH/gL/UL128/UL130/UL131A. Such a pentameric complex mediates the entry of hCMV into epithelial cells, endothelial cells, monocytes, and dendritic cells. FIG. 1C, which is adapted from Macagno et al. (2010) J. Virology 84(2):1005-13 shows the hCMV pentameric complex (gH/gL/UL128/UL130/UL131A) further in complex with antibodies specific for the protein components of the pentameric complex: 8I21 (anti-pentamer), 3G16 (anti-gH), 15D8 (anti-UL 128), 7113 (anti-UL128/UL130/UL131A), and 10P3 (anti-gL).

FIGS. 2A-2D show that delivery of pre-mixed mRNAs encoding the various subunits of hCMV pentamer leads to surface expression of the pentameric complex in HeLa cells. FIG. 2A shows the surface expression of gH. FIG. 2B shows the surface expression of UL128/UL130/UL131A. FIG. 2C shows the surface expression of UL128. FIG. 2D shows the surface expression of the pentamer. The indicated subunits were detected by monoclonal antibodies. Data in bar graphs represent meant standard deviation (s.d.).

FIGS. 3A-3B show the surface expression of hCMV petameric complex (PC). Hela cells were transfected with mRNAs for all five subunits of the pentameric complex or lacking one of the subunits, as indicated. After 24 hr, cells were stained with anti-PC antibody 8I21 and analyzed by flow cytometry. Representative flow cytometry plots (FIG. 3A) show PC surface expression. A bar graph (FIG. 3B) shows percent PC surface expression. hCMV pentameric complex was not observed to be expressed on the cell surface in the absence of one of the core subunits. Surface expression of the pentamer was only detected at high levels when all the core subunits were expressed.

FIGS. 4A-4B shows the dimerization of gH-gL is sufficient to lead to surface expression of gH. The anti-gH antibody (3G16) was used for the detection of gH on the cell surface. When gH and gL were co-expressed, a similar level of gH was detected on the surface of HeLa cells as when all subunits in the pentameric complex were expressed. When gH was expressed alone, very little gH was detected on the surface of the transfected HeLa cells.

FIGS. 5A-5D show the intracellular and surface expression of hCMV antigen gB. The mRNA encoding gB was expressed both intracellularly and on the cell surface (FIGS. 5A-5C). Both gB precursor and the proteolytically processed, mature gB, were detected by anti-gB antibodies in an immunoblot (FIG. 5D). “*” indicates that the lane was overloaded.

FIG. 6 shows an immunogenicity study of the hCMV pentameric complex mRNA vaccine constructs. Mice were vaccinated according to the vaccination schedule with indicated dosages of the mRNAs. High titers of anti-pentamer antibodies were detected in mice serum following the immunization. Different formulations of the pentamer mRNAs produced comparable levels of antibodies. A third immunization did not lead to boosting of antibody production.

FIG. 7 shows an immunogenicity study of the hCMV gB mRNA vaccine construct, with or without the pentameric complex mRNA constructs. gB mRNA constructs produced similar IgG titers as the gB protein/MF59 antigens after 3 immunizations. A boost in IgG production was observed after the third immunization. Addition of pentameric mRNA constructs did not interefere with the induction of anti-gB IgG.

FIG. 8 shows a neutralization study of the hCMV pentameric complex mRNA vaccine constructs in the epithelial cell line ARPE-19. IE1 staining in infected ARPE-19 cells is demonstrated. Immunization with hCMV pentameric complex mRNA vaccine constructs elicits highly potent neutralizing antibodies in mice. Neutralizing antiboey titer (1:25600) in mice serum at day 41 (3 weeks post second immunization) was able to neutrzalize the hCMV clinical isolate VR1814 in ARPE-19 cells.

FIG. 9 shows a measurement of hCMV neutralization IgG tiers in ARPE-19 cells infected with the hCMV clinical isolate strain VR1814. See also Table 5.

FIGS. 10A-10B show the surface expression in HeLa cells of the hCMV pentameric complex (gH-gL-UL128-UL130-UL131A) encoded by the first-generation pentameric constructs described herein (referred to as “version 1” or “V1”) and second-generation pentameric constructs also described herein (referred to as “version 2” or “V2”). The sequences of the mRNAs within the second generation constructs are provided in Table 6, corresponding to SEQ ID NOs: 58-69. FIGS. 10A and IOC show the results of a fluorescence-activated cell (FACS) sorting experiment detecting the surface expression of the pentameric complex using the 8I21 (anti-pentamer) antibodies. Surface expression of the pentameric complex is indicated by the emerging fluorescent cell population. FIGS. 10B and 10D shows the quantification of the FACS experiment.

FIGS. 11A-11E depict Western blots showing the expression of the subunits of the hCMV pentameric complex (gH, gL, UL128, U1130, and UL131A) encoded by the first generation pentameric constructs described herein (referred to as “version 1” or “V1”) and second-generation pentameric constructs also described herein (referred to as “version 2” or “V2”). Polyclonal antibodies against the various subunits were used for detection. β-actin serves as a loading control.

FIG. 12 shows that immunization with the pentameric mRNA complex elicits high titers of antibodies that are maintained up to several months. An immunogenicity study of the second generation hCMV pentameric complex mRNA vaccine constructs is shown. Balb/c mice were vaccinated according to the vaccination schedule with indicated dosages of the mRNAs (lower panel). Mice serum IgG titers were measured at days 20, 41, 62, 92, and 123 post immunization. hCMV pentamer coated plates were used to measure the serum IgG titer. High titers of anti-pentamer antibodies were detected in the serum of the immunized mice.

FIG. 13 shows that hCMV mRNA vaccines encoding the pentamer elcited higher neutralizing antibody titers in mice than CytoGam®, a hyperimmune serum used clinically for prophylaxis of hCMV. Balb/c mice were vaccinated according to the vaccination schedule with indicated dosages of the mRNAs (lower panel). Neutralizing antibody titers in mice serum were measured at days 42, 122, 152, and 182 post immunization, with ARPE-19 epithelial cells infected with the hCMV clinical isolate VR1814. High titers of neutralizing antibodies induced by the hCMV pentameric complex mRNA vaccine were maintained up to 6 months.

FIG. 14 is a graph showing the neutralizing antibody titers induced in mice by the hCMV pentameric complex mRNA vaccine constructs. Balb/c mice were vaccinated according to the vaccination schedule with indicated dosages of the mRNAs (lower panel). Neutralizing antibody titers in mice serum were measured at days 42, 62, and 182 post immunization, with HEL299 fibroblast cells infected with 500-2000 pfu of hCMV AD169 strain.

FIG. 15 is a schematic representation of pentametic subunits linked by a self-cleaving 2A peptide (e.g., as described in Kim et al., PLoS ONE 6(4): e18556, 2011).

FIG. 16 is a Western blot showing that gH and gL linked by the 2A peptide underwent efficient self-cleavage to generate individual gH and gL subunits.

FIG. 17 shows that the individual gH and gL subunits generated from self-cleavage of the 2A peptide linked were able to dimerize and translocate to the cell surface.

FIGS. 18A-B demonstrates high and sustained titers of anti-pentamer binding and neutralizing antibodies in mice. FIG. 18A depicts a graph showing anti-pentamer antibody titers. Equimolar and equal mass formulations of the pentameric mRNAs were compared and were found to be equally effective. FIG. 18B depicts a graph showing neutralizing titers measured on ARPE19 epithelial cells infected with hCMV strain VR1814. Equimolar and equal mass formulations of the pentameric mRNAs were compared and were found to be equally effective. Neutralizing titers were found to be approximately 25 fold higher than CytoGam®.

FIG. 19A-C demonstrate that neutralization activity against epithelial cell infection is dependent on anti-pentamer antibodies. FIGS. 19A-C show that the depleting protein was either the pentamer or a gH/gL dimer. FIG. 19B and FIG. 19C depict graphs showing neutralization. FIG. 19B shows neutralization by sera from mice immunized with the pentamer or with a gH/gL dimer. FIG. 19C shows neutralization by CytoGam® combined with the pentamer or with a gH/gL.

FIGS. 20A-20B are graphs showing the immunogenicity of second generation hCMV mRNA vaccine constructs formulated with Compound 25 lipids. The second generation mRNA constructs encoding the pentamer and gB induced pentamer-specific antibodies (FIG. 20A) and gB-specific antibodies (FIG. 20B) as early as 20 days post first immunization. The pentamer-specific and gB-specific antibody titers continue to increase in mice after the boost dose.

FIGS. 21A-21C show an analysis of the CD8 IFNγ responses in Balb/c mice splenic lymphocytes stimulated with a pp65-IE1 peptide pool. The mice were immunized with pp65-IE-1 or pp65-IE-1+gB mRNA vaccine constructs. pp65-IE1 mRNA induced specific CD8 IFNγ response. FIG. 21A shows the pp65-IE1 peptide pool stimulated CD8 IFNγ response, as indicated by the emerging IFNγ+ cell population in FACS experiments. FIGS. 21B-21C show the quantification of IFNγ response in splenocytes of mice immunized with different mRNA vaccine constructs.

FIGS. 22A-22C show an analysis of the CD4 IFNγ responses in Balb/c mice splenic lymphocytes stimulated with a pp65-IE1 peptide pool. The mice were immunized with pp65-IE-1 or pp65-IE-1+gB mRNA vaccine constructs. pp65-IE1 mRNA induced specific CD4 IFNγ response. FIG. 22A shows the pp65-IE1 peptide pool stimulated CD4 IFNγ response, as indicated by the emerging IFNγ+ cell population in FACS experiments. FIGS. 22B-22C show the quantification of IFNγ response in splenocytes of mice immunized with different mRNA vaccine constructs.

FIG. 23 is a graph showing that addition of other hCMV antigens (gB and/or pp65-IE1) does not interfere with the production of high titer pentamer-specific antibodies from the hCMV pentameric complex mRNA vaccine. Balb/c mice were vaccinated according to the vaccination schedule with indicated dosages of the mRNAs (lower panel). Mice serum IgG titers were assayed on day 41 post immunization on pentamer coated plates. Similar levels of pentamer-specific IgG titers were produced with addition of gB antigen and/or pp65-IE1 antigen.

FIG. 24 is a graph showing that gB-specific antibody titers induced by gB mRNA vaccine were maintained in the presence of the hCMV pentamer and pp65-IE1. Balb/c mice were vaccinated according to the vaccination schedule with indicated dosages of the mRNAs (lower panel). Mice serum IgG titers were assayed on day 41 post immunization on gB coated plates. The presence of the pentamer and pp65-IE1 did not interfere with the induction of gB-specific IgG titer.

FIGS. 25A-25C are graphs showing the analysis of T cell responses in Balb/c mice splenic lymphocytes stimulated with a pentamer peptide library or a pp65-IE1 peptide pool. The mice were immunized with hCMV pentamer, gB, and pp65-IE1 mRNA vaccines. FIG. 25A shows the CD8 (left panel) and CD4 (right panel) response induced by a pentamer library. The mRNA vaccine used to immunize the mice was pentamer (5 μg):gB (5 μg): pp65-IE1 (2 μg). FIG. 25B shows the CD8 (left panel) and CD4 (right panel) response induced by a pp65-IE1 peptide pool. The mRNA vaccine used to immunize the mice was pentamer (1 μg):gB (1 μg): pp65-IE1 (1 μg). FIG. 25C shows the CD8 (left panel) and CD4 (right panel) response induced by a pp65-IE1 peptide pool. The mRNA vaccine used to immunize the mice was pentamer (5 μg):gB (5 μg): pp65-IE1 (2 μg).

FIG. 26 shows an immunogenicity study of hCMV mRNA vaccines encoding the hCMV pentamer combined with gB or the pentamer combined with both gB and pp65-IE1. Balb/c mice were vaccinated intramuscularly according to the vaccination schedule with indicated dosages of the mRNAs (lower panel). Mice serum IgG titers were assayed on day 41 post immunization on pentamer coated plates. Similar levels of pentamer-specific IgG titers were produced with addition of gB and/or pp65-IE1.

FIG. 27 shows an immunogenicity study of the hCMV mRNA vaccines encoding the hCMV pentamer combined with gB or the pentamer combined with gB and pp65-IE1. Balb/c mice were vaccinated intramuscularly according to the vaccination schedule with indicated dosages of the mRNAs (lower panel). Mice serum IgG titers were assayed on day 41 post immunization on gB coated plates. Similar levels of gB-specific IgG titers were produced with addition of the pentamer and/or pp65-IE1.

FIG. 28 depicts a graph showing the neutralizing antibody titers induced in mice by the hCMV mRNA vaccine constructs encoding the hCMV pentamer, gB, and pp65-IE1. Balb/c mice were vaccinated intramuscularly according to the vaccination schedule with indicated dosages of the mRNAs (lower panel). Neutralizing antibody titers in mice serum were measured at days 41 post immunization, with either ARPE-19 cells infected with ˜1000 pfu of the hCMV VR1814 strain, or HEL200 cells infected with ˜1000 pfu of the AD169 strain. The results show that the hCMV pentamer mRNA vaccine induced comparable or higher neutralization titers in mice that CytoGam®, which is a hyperimmune serum used clinically for prophylaxis of hCMV.

FIG. 29 is a graph showing the immunogenicity of hCMV mRNA vaccines formulated in lipid nanoparticles (Compound 25 and MC3). High titers of pentamer-specific antibodies were generated in Cynomolgus macaques following immunization with hCMV mRNA vaccines encoding the pentamer, gB, and pp65-IE1. Compound 25 formulation and MC3 formulation induced comparable antibody titers at high doses.

FIGS. 30A-30B is a graph showing an analysis of the neutralizing antibody titers induced by the hCMV mRNA vaccines formulated in lipid nanoparticles (Compound 25 and MC3). A 100 μg total dose of the mRNA vaccines formulated with Compound 25 lipids or MC3 lipids exhibited comparable ability to induce neutralizing antibodies against CMV infection as CytoGam®. FIG. 30A shows the results of neutralization assays performed on ARPE-19 epithelial cells infected with hCMV strain VR1814. FIG. 30B shows the results of neutralization assays performed on HEL299 fibroblast cells infected with hCMV strain AD169.

FIGS. 31A and 31B are graphs showing that low doses of hCMV mRNA vaccine encoding the pentamer, pentamer+gB, or pentamer+gB+pp65-IE1 formulated with MC3 lipids elicits neutralizazing antibody titers that are higher or equivalent to CytoGam®. FIG. 31A shows the results of neutralization assays performed on ARPE-19 epithelial cells infected with hCMV strain VR 1814. FIG. 31B shows the results of neutralization assays performed on HEL299 fibroblast cells infected with hCMV strain AD169.

FIG. 32 is a graph showing that immunization with hCMV pentameric complex mRNA vaccine either alone or in combination with mRNAs encoding other antigens elicits similar levels of binding antibodies that are maintained over time.

FIG. 33 is a graph showing the hCMV multivalent vaccine induced high titers of anti-gB antibodies in mice.

FIGS. 34A and 34B are graphs showing that immunization with multivalent hCMV vaccine in Cynomolgus macaques elicited potently neutralizing antibodies with either Compound 25 or MC3 lipid formulation. FIG. 34A shows the results of neutralization assays performed on ARPE-19 epithelial cells infected with hCMV strain VR1814. FIG. 34B shows the results of neutralization assays performed on HEL299 fibroblast cells infected with hCMV strain AD169.

FIG. 35 is a graph showing that a 3 μg total dose of HCMV mRNA vaccine constructs encoding the pentameric complex elicited higher neutralization antibody titers than CytoGam®, a hyperimmune serum used clinically for prophylaxis of hCMV.

FIG. 36 depicts graphs showing the T cell response elicited by multivalent hCMV mRNA vaccine constructs encoding the pentamer, in mice. The mRNA vaccines constructs were formulated in Compound 25 lipid particles. Formulations tested included: mRNA encoding the pp65mut alone; mRNA encoding the pp65mut+mRNA encoding gB combined with mRNA encoding the pentamer; or mRNA encoding the pp65mut+mRNA encoding gB+the mRNA encoding the pentamer. A robust T cell response to the pentamer was elicited by the multivalent hCMV mRNA vaccine.

FIGS. 37A and 37B are graphs showing antibody titers elicited by hCMV mRNA vaccine constucts encoding hCMV pentamer (5 μg) and gB (1 μg), or constructs encoding hCMV pentamer (5 μg), gB (1 μg), and pp65mut (2 μg). The mRNA vaccine constructs were either unmodified (C1) or contained N1-methylpseudouridine chemical modification (C2) and were formulated in MC3 lipid particles. Mice were immunized with two doses (one primary dose and one booster dose on day 21 post primary dose) of the hCMV mRNA vaccines and sera were collected at days 21 and 43 post primary dose. The sera were analyzed on plates coated with hCMV pentamer (FIG. 37A) or gB (FIG. 37B). The hCMV mRNA vaccine constructs elicited antibody titers specific for hCMV pentmer and gB, and the antibody titers increased after the boost dose.

FIGS. 38A and 38B are graphs showing antibody titers elicited by hCMV mRNA vaccine constructs encoding hCMV pentamer (5 μg) and gB (1 μg), or constructs encoding hCMV pentamer (5 μg), gB (1 μg), and pp65mut (2 μg). The mRNA vaccine constructs were either unmodified (C1) or contained N1-methylpseudouridine chemical modification (C2), and were formulated in compound 25 lipid particles. Mice were immunized with two doses (one primary dose and one booster dose on day 21 post primary dose) of the hCMV mRNA vaccines and sera were collected at days 21 and 43 post primary. The sera were analyzed on plates coated with hCMV pentamer (FIG. 38A) or gB (FIG. 38B). The hCMV mRNA vaccine constructs elicited antibody titers specific for hCMV pentmer and gB and the antibody titers increased after the boost dose.

FIGS. 39A and 39B are graphs showing the neutralizing antibody titers against infection of hCMV in fibroblast cells (FIG. 39A) and epithelial cells (FIG. 39B). The mRNA vaccine constructs were either unmodified (C1) or contained N1-methylpseudouridine chemical modification (C2), and were formulated in either MC3 lipid particles or compound 25 lipid particles. Mice were immunized as described in FIGS. 37A and 37B. Mice sera were collected 20 days after the booster dose. The neutralizing antibody titers in the sera of immunized mice were measured on HEL299 cells infected with about 1000 pfu of hCMV AD169 strain (FIG. 39A) or were measured on ARPE-19 cells infected with about 1000 pfu of hCMV VR1814 strain (FIG. 39B). All mRNA vaccine constructs formulated with either MC3 or compound 25 lipid particles elicited neutralizing antibody titers against hCMV infection.

FIGS. 40A-40C are graphs showing T-cell responses (CD4+ T-cell response and CD8+ T-cell response) elicited by hCMV mRNA vaccine constructs encoding hCMV pentamer (5 μg), gB (1 μg), and pp65mut (2 μg). The mRNA vaccine constructs were either unmodified (C1) or contained N1-methylpseudouridine chemical modification (C2), and were formulated in MC3 lipid particles. FIG. 40A shows the T-cell responses elicited by the hCMV mRNA vaccine constructs against hCMV pentamer. FIG. 40B shows the T-cell responses elicited by the hCMV mRNA vaccine constructs against pp65. FIG. 40C shows the T-cell responses elicited by the hCMV mRNA vaccine constructs against gB.

FIGS. 41A-41C are graphs showing T-cell responses (CD4+ T-cell response and CD8+ T-cell response) elicited by hCMV mRNA vaccine constructs encoding hCMV pentamer (5 μg), gB (1 μg), and pp65mut (2 μg). The mRNA vaccine constructs were either unmodified (C1) or contained N1-methylpseudouridine chemical modification (C2), and were formulated in compound 25 lipid particles. FIG. 41A shows the T-cell responses elicited by hCMV mRNA vaccine constructs against hCMV pentamer. FIG. 41B shows the T-cell responses elicited by the hCMV mRNA vaccine constructs against pp65. FIG. 41C shows the T-cell responses elicited by the hCMV mRNA vaccine constructs against gB.

FIGS. 42A-42C show intracellular and cell surface expression analysis of gB by flow cytometry. HeLa cells were either untransfected (control) or transfected with gB mRNA; after 24 hr, the cells were either fixed and permeabilized or not fixed and stained with mouse monoclonal anti-gB antibody. Intracellular expression (FIG. 42A) and surface expression (FIG. 42B) were analyzed by flow cytometry. Representative flow cytometry plots (top) and bar graphs (bottom) depict percent intracellular gB expression. FIG. 42C depicts a Western blot showing expression of gB.

FIGS. 43A-43B are graphs showing expression of surface pentameric complex formation in cells transfected with the indicated mRNAs in equal mass ratios. HeLa cells were transfected with equal mass ratios of mRNAs for all five subunits of the pentametic complex or lacking one of the subunits, as indicated. Cell surface expression of the pentameric complex was analyzed by flow cytometry. FIG. 43A shows flow cytometry plots showing surface expression of the pentameric complex. FIG. 43B is a bar graph. The data in the bar graph represents mean t standard deviation.

FIGS. 44A-44E demonstrate the neutralizing antibodies and specificity of antibodies in sera of mice immunized with the hCMV mRNA vaccines. FIG. 44A is a schematic of the vaccination regimen in mice showing days of dosing, blood draws, and spleen harvest. FIGS. 44B and 44C show neutralizing titers in sera from mice immunized with the indicated doses and mRNA groups. All mRNAs were present at equal mass in the various vaccine groups. Numbers in parentheses depict the dose of each antigen. PD1, PD2, and PD3 refer to postdose 1, postdose 2, and postdose 3, respectively. Shown are neutralization titers against VR1814 infection in ARPE-19 epithelial cells (FIG. 44B) and against AD169 infection in HEL299 fibroblast cells (FIG. 44C). FIGS. 44D and 44E show specificity of neutralizing antibodies in sera of mice immunized with hCMV mRNA vaccine. Mouse immune serum was preincubated with 5 μg of purified gB, gH/gL, or pentameric complex protein prior to performance of neutralization assays. Also shown are NT50 titers against epithelial (FIG. 44D) and fibroblast cell (FIG. 44E) infection. LOD refers to lower limit of detection; and CG refers to Cytogam. Results represent the mean k standard deviation in scatter and bar graphs. N=5 for all groups. Statistical analysis was done using the Kruskal-Wallis test and Dunn's multiple comparison test (*p<0.05).

FIGS. 45A-45B are graphs showing antibody responses in mice immunized with hCMV multivalent mRNA vaccines. Anti-pentamer (FIG. 45A) and anti-gB (FIG. 45B) binding titers in sera from BALB/c mice immunized with the indicated doses and mRNA groups are shown. Numbers in parentheses indicate individual doses of each antigen. Also shown is the total dose of each vaccine. The dotted line represents positive cut-off values. N=5 for all groups.

FIGS. 46A-46E demonstrate the neutralizing activity and specificity of antibodies in sera of non human primates (NHP) vaccinated with hCMV mRNA vaccine. FIG. 46A is a schematic of the vaccination regimen and blood draws in NHPs. Neutralizing titers are measured as described in FIGS. 44A-44E in the sera of NHPs that received two doses of the indicated vaccines. All mRNAs were present at equal mass in the various vaccine groups; the total dose is shown in parentheses. NT50 was measured on ARPE-19 cells infected with VR1814 strain (left, 400 μg and 25 μg dose; right, 100 μg dose, FIG. 46B) and HEL 299 cells infected with AD169 strain (left, 400 μg and 25 μg doses; right, 100 μg dose, FIG. 46C). Specificity of antibodies elicited by immunization of NHPs with HCMV antigens was assessed. NHP immune serum and Cytogam were preincubated with 5 μg of purified gB, g/gL, or the pentameric complex protein prior to performing neutralization assays. NT50 titers against epithelial (FIG. 46D) and fibroblast (FIG. 46E) cell infection are shown as mean±standard deviation, N=3, for each group. Statistical analysis was done using the Kruskal-Wallis test and Dunn's multiple comparison test (*p<0.05).

FIGS. 47A-47B show antibody responses in NHPs immunized with hCMV multivalent vaccines. Anti-pentameric complex (PC) (left, 400 μg and 25 μg doses; right, 100 μg dose. FIG. 47A) and anti-gB (left, 400 μg and 25 μg doses; right, 100 μg dose, FIG. 47B) binding titers in sera of NHPs immunized with the indicated doses of the various LNP/mRNA formulations are shown. The dotted line represents positive cut-off values. Results show mean±standard deviation, N=3, for each group.

FIGS. 48A-48F show differential T cell responses to pp65 and the petameric complex in hCMV mRNA vaccine. FIGS. 48A and 48B show T cell responses to pp65-IE1. One week following boost, CD4 (FIG. 48A) and CD8 (FIG. 48B) T cells secreting IFNγ in response to pp65-IE1 peptide pools were measured by ICS and analyzed by flow cytometry. pp65-IE1 was present at a dose of 2 μg in both vaccine groups. FIGS. 48C and 48D show pp65-specific CD4 (FIG. 48C) and CD8 (FIG. 48D) T cell responses. FIGS. 48E and 48F show pentamer-specific CD4 (FIG. 48E) and CD8 (FIG. 48F) T cell responses. One week postboost, splenocytes from the indicated groups were stimulated either with pp65 (FIGS. 48C and 48D) or pentameric complex (PC) (FIGS. 48E and 48F) peptide libraries, and polyfunctional (IFNγ, TNF-α, IL-2) T cell responses were measured by ICS and analyzed by flow cytometry. Scatter plots represent mean±standard deviation. For FIGS. 48C-48E, the doses of pentameric complex, gB, and pp65 were 8 μg, 2 μg, and 2 μg, respectively, wherever applicable. N=5 for all groups. Statistical analysis was done using the two-tailed Mann-Whitney U test (*p<0.05, **p<0.01, ****p<0.0001).

FIGS. 49A-49E show that heterologous prime boost vaccine regimen restores pp65 specific T cell responses. FIG. 49A is a schematic of heterologous prime boost dosing schedule. FIGS. 49B-49E show pp65-specific (FIGS. 49B and 49C) and pentamer-specific (FIGS. 49D and 49E) T cell responses in mice vaccinated with the indicated hCMV mRNA antigens. Polyfunctional T cell responses were measured as described in FIGS. 48A-48F. Shown are pp65-specific CD4 (FIG. 49B) and CD8 (FIG. 49C) and pentameric complex (PC)-specific CD4 (FIG. 49D) and CD8 (FIG. 49E) T cell responses. Scatter plots represent mean t standard deviation, N=5, for all groups. Statistical analysis was done using the Kruskal-Wallis test and Dunn's multiple comparison test (*p<0.05, **p<0.01).

FIGS. 50A-50B show T cell responses in mice vaccinated with pp65^{ΔP} and pp65^{ΔP-IE1}. CD4 (FIG. 50A) and CD8 T (FIG. 50B) cells secreting IFNγ in response to pp65 were measured by ICS and analyzed by Flow Cytometry. A 2 μg dose of mRNA was used for each vaccine group. Statistical analysis was done using the two-tailed Mann Whitney U test (*p<0.05, **p<0.01). N=5 for each group.

FIGS. 51A-51D show the T cell responses to various hCMV antigens. FIG. 51A shows pp65-specific CD4 and CD8 T cells that secrete IL2. FIG. 51B shows pentamer-specific T cells that secrete IL2. FIGS. 51C-51D show polyfunctional CD4 (FIG. 51C) and CD8 (FIG. 51D) T cell responses to gB antigen. T cell responses were measured as described in FIG. 5. Scatter plots represent mean±standard deviation, N=5, for all groups. Statistical analysis was done using the two-tailed Mann Whitney U test (*p<0.05).

FIG. 52 shows the expression of gB in HEK 293 cells using codon-optimized gB mRNA variants. Compared to the wild type gB mRNA, several of the codon-optimized variants (Var #1-Var #4, Var #9, and Var #10) led to enhanced expression in HEK293 cells, among which Var #4 had the highest expression level. * indicates truncated proteins due to out of frame AUGs or background bands.

### DETAILED DESCRIPTION

Embodiments of the present disclosure provide RNA (e.g., mRNA) vaccines that include polynucleotide encoding a human cytomegalovirus (HCMV) antigen. Demonstrated herein is an HCMV vaccine that elicits broad and durable neutralizing antibodies as well as robust T cell responses. The human cytomegalovirus (HCMV) is a ubiquitous double-stranded DNA virus belonging to the Herpes virus family. HCMV is made up of a DNA core, an outer capsid and covered by a lipid membrane (envelope) which incorporates virus specific glycoproteins. The diameter is around 150-200 nm. Genomes are linear and non-segmented, around 200 kb in length. Viral replication is nuclear, and is lysogenic. Replication is dsDNA bidirectional replication.

HCMV can infect a wide range of mammalian cells, which correlates with its ability to infect most organs and tissues. Entry into the host cell is achieved by attachment of the viral glycoproteins to host cell receptors, which mediates endocytosis. HCMV displays a broad host cell range, with the ability to infect several cell types, such as endothelial cells, epithelial cells, smooth muscle cells, fibroblasts, leukocytes, and dendritic cells. This broad cellular tropism suggests that HCMV may bind a number of receptors or a common surface molecule.

HCMV encodes several surface glycoproteins that are important for viral attachment and entry into different cell types. Entry into fibroblast cells is mediated by the core herpesvirus fusion machinery comprising gB and the gH/gL/gO ternary complex (Vanarsdall and Johnson, 2012; Vanarsdall et al., 2008, incorporated herein by reference). The pentameric complex (PC), composed of gH/gL/UL128/UL130/UL131A (Hahn et al., 2004; Ryckman et al., 2008; Wang and Shenk, 2005b, incorporated herein by reference), mediates entry into endothelial cells, epithelial cells, and myeloid cells. The majority of neutralizing antibodies are directed against envelope glycoproteins (Britt et al., 1990; Fouts et al., 2012; Macagno et al., 2010; Marshall et al., 1992, incorporated herein by reference), whereas robust T cell responses are directed against the tegument protein pp65 and nonstructural proteins such as IE1 and IE2 (Blanco-Lobo et al., 2016; Borysiewicz et al., 1988; Kern et al., 2002, incorporated herein by reference).

HCMV envelopment is very complicated and comprises more than 20 glycoproteins which may be the reason for broad cellular tropism of HCMV. HCMV particles contain at least four major glycoprotein complexes, all of which are involved in HCMV infection, which requires initial interaction with the cell surface through binding to heparin sulfate proteoglycans and possibly other surface receptors.

The gCI complex is comprised of dimeric molecules of the glycoprotein gB. Each 160-kDa monomer is cleaved to generate a 116-kDa surface unit linked by disulfide bonds to a 55-kDa transmembrane component. Some antibodies immunospecific for gB inhibit the attachment of virions to cells, whereas others block the fusion of infected cells, suggesting that the gB protein might execute multiple functions at the start of infection. Studies have confirmed that glycoprotein B (gB) facilitates HCMV entry into cells by binding receptors and mediating membrane fusion. Several cellular membrane proteins interact with gB, which interactions likely facilitate entry and activate cellular signaling pathways.

The gCII complex is the most abundant of the glycoprotein complexes and is a heterodimer consisting of glycoproteins gM and gN. The complex binds to heparan sulfate proteoglycans, suggesting it might contribute to the initial interaction of the virion with the cell surface. It may also perform a structural role during virion assembly/envelopment, similar to the gM-gN complex found in some α-herpesviruses.

The gCII complex is a trimer comprised of glycoproteins gH, gL, gO which are covalently linked by disulfide bonds. All known herpesviruses encode gH-gL heterodimers, which mediate fusion of the virion envelope with the cell membrane. Antibodies specific for human CMV gH do not affect virus attachment but block penetration and cell-to-cell transmission. A gO-deficient mutant of HCMV (strain AD169) shows a significant growth defect.

HCMV proteins UL128, UL130, and UL131A assemble with gH and gL proteins to form a heterologous pentameric complex, designated gH/gL/UL128-131A, found on the surface of the HCMV. Natural variants and deletion and mutational analyses have implicated proteins of the gH/gL/UL128-131A complex with the ability to infect certain cell types, including for example, endothelial cells, epithelial cells, and leukocytes.

HCMV enters cells by fusing its envelope with either the plasma membrane (fibroblasts) or the endosomal membrane (epithelial and endothelial cells). HCMV initiates cell entry by attaching to the cell surface heparan sulfate proteoglycans using envelope glycoprotein M (gM) or gB. This step is followed by interaction with cell surface receptors that trigger entry or initiate intracellular signaling. The entry receptor function is provided by gH/gL glycoprotein complexes. Different gH/gL complexes are known to facilitate entry into epithelial cells, endothelial cells, or fibroblasts. For example, while entry into fibroblasts requires gH/gL heterodimer, entry into epithelial and endothelial cells requires the pentameric complex gH/gL/UL128/UL130/UL131 in addition to gH/gL. Thus, different gH/gL complexes engage distinct entry receptors on epithelial/endothelial cells and fibroblasts. Receptor engagement is followed by membrane fusion, a process mediated by gB and gH/gL. Early antibody studies have supported critical roles for both gB and gH/gL in HCMV entry. gB is essential for entry and cell spread. gB and gH/gL are necessary and sufficient for cell fusion and thus constitute the “core fusion machinery” of HCMV, which is conserved among other herpesviruses.

Thus, the four glycoprotein complexes play a crucial role in viral attachment, binding, fusion and entry into the host cell.

Studies involving the gH/gL/UL128-131A complex have shown that HCMV glycoproteins gB, gH, gL, gM, and gN, as well as UL128, UL130, and UL131A proteins, are antigenic and involved in the immunostimulatory response in a variety of cell types. Moreover, UL128, UL130, and UL131A genes are relatively conserved among HCMV isolates and therefore represent an attractive target for vaccination. Furthermore, recent studies have shown that antibodies to epitopes within the pentameric gH/gL/UL128-131 complex neutralize entry into endothelial, epithelial, and other cell types, thus blocking the ability of HCMV to infect several cell types.

HCMV envelope glycoprotein complexes (gCI, II, III, gH/gL/UL128-131A) represent major antigenic targets of antiviral immune responses. Embodiments of the present disclosure provide RNA (e.g., mRNA) vaccines that include polynucleotide encoding a HCMV antigen, in particular an HCMV antigen from one of the HCMV glycoprotein complexes. Embodiments of the present disclosure provide RNA (e.g., mRNA) vaccines that include at least one polynucleotide encoding at least one HCMV antigenic polypeptide. The HCMV RNA vaccines provided herein may be used to induce a balanced immune response, comprising both cellular and humoral immunity, without many of the risks associated with DNA vaccines and live attenuated vaccines.

The entire contents of International Application No. PCT/US2015/027400 (WO 2015/164674), entitled “Nucleic Acid Vaccines,” is incorporated herein by reference.

### HCMV Vaccine for Transplant Patients

Although HCMV infection is benign in most healthy adults, it can sometimes result in serious diseases, such as retinitis, in immunocompromised patients, e.g., an organ transplant recipient. An “immunocomprised patient” refers to a patient who does not have the ability to respond normally to an infection due to an impaired or weakened immune system. This inability to fight infection can be caused by a number of conditions including illness and disease (e.g., in some embodiments, diabetes, HIV), malnutrition, and drugs.

The control of HCMV infection in immunocomprised patients, e.g., organ transplant recipients who are receiving immunosuppressive drugs to suppress their adaptive immune systems, is associated with preserved cellular immune responses, e.g., T cell responses involving CD4+, CD8+, and NK T cells (Riddell et al., Semin. Respir. Infect. 10:199-208, 1995). HCMV antigens that elicit T cell responses (e.g., CD8+ responses) include, without limitation, the major tegument protein pp65, and the early-immediate proteins such as IE1 (e.g., in Khan et al., J. Infect. Dis. 185, 1025-1034, 2002). In some instances, T cell responses specific to the glycoprotein gB can be elicited (Borysiewicz et al., J. Exp. Med. 168, 919-931, 1988). CD4+ response is present in almost all individuals infected with HCMV. (Kern, F., et al., J. Infect. Dis. 185:1709-1716 (2002)).

In some embodiments, the immunocomprised patient is an organ transplant recipient. An “organ transplant recipient” refers to a subject who has received or will receive an organ transplant. As used herein, an “organ transplant” refers to the moving of an organ or tissue from a donor to a recipient. In some embodiments, a donor and a recipient are different subjects. In other embodiments, a donor and a recipient are the same subject. Donors and recipients can be human or non-human subjects. For example, in some embodiments, a donor and a recipient are both human subjects. In other embodiments, a donor is a non-human subject and a recipient subject is a human subject. In other embodiments, a donor is a human subject and a recipient is a non-human subject. In other embodiments, a donor and a recipient are both non-human subjects.

In some embodiments, the organ transplant recipient is a solid organ transplant (SOT) recipient. Solid organs/tissue that may be transplanted include, without limitation, heart, kidney, liver, lungs, pancreas, intestine, thymus, bones, tendons, cornea, skin, heart valves, nerves and veins. In some embodiments, the organ transplant recipient is a hematopoietic cell transplant (HCT) recipient. “Hematopoietic cell transplantation (HCT)” refers to the intravenous infusion of hematopoietic cells to a recipient. Hematopoietic cells may be from, e.g., bone marrow, peripheral blood, amniotic fluid, and umbilical cord blood. Bone marrow transplantation is a common type of hematopoietic stem cell transplantation. Hematopoietic cells can be transplanted from a donor to a recipient. The donor and recipient can be the same subject or different subjects.

The donor or recipient of a transplantation can be HCMV seropositive or seronegative. “Seropositive” means the individual (e.g., the transplant donor and/or the recipient) has had a past HCMV infection and HCMV IgG can be detected in his/her blood. Being “seropositive” does not necessarily mean that there is live, replicating HCMV in the blood of the subject. An individual who has not had a past HCMV infection does not have HCMV specific IgG in his/her blood, and is therefore “seronegative.”

Without appropriate prophylactic measures, the seronegative recipient of an organ from a seropositive donor can be at high risk (>60%) of developing CMV disease. IgG detection can be used to diagnose donor seropositivity since donors generally have intact humoral responses. In some embodiments, the recipient is seropositive but the HCMV is latent, and the HCMV is reactivated after the transplantation.

“Latent,” or “latency” refers to a phase in certain viruses' life cycles in which, after initial infection, proliferation of virus particles ceases. However, the viral genome is not fully eradicated. As a result, the virus can reactivate and begin producing large amounts of viral progeny without the host being infected by new outside virus. A virus can potentially stay within a host indefinitely. In some instances, a latent virus can be reactivated via external activators (i.e. sunlight, stress) to cause an acute infection.

Transplant recipients disclosed herein include subjects that are immunocompromised and subjects that are not immunocompromised. HCMV-associated diseases in organ transplant recipients can affect most organs of the body, and can result in, e.g., fever, pneumonia, hepatitis, encephalitis, myelitis, colitis, uveitis, retinitis, neuropathy, Guillain-Barre syndrome, meningoencephalitis, pericarditis, myocarditis, thrombocytopenia, hemolytic anemia, deadly pneumonitis, esophagitis, leukopenia, infections, and complications in organ transplant.

Aspects of the present disclosure provide safe and effective HCMV vaccines and methods to protect subjects, including immunocomprised organ transplant recipients, against HCMV infection. HCMV vaccines disclosed herein include RNA vaccines (e.g., mRNA vaccines) that encode at least one HCMV antigenic polypeptide, or an immunogenic fragment thereof. In some embodiments, the antigenic polypeptides or immunogenic fragments encoded by the HCMV RNA vaccine (e.g., mRNA vaccine) of the present disclosure are selected from gB, gH, gL, gO, gM, gN, UL83, UL123, UL128, UL130, UL131A, pp65 and IE1 antigens. In some embodiments, the HCMV RNA vaccine (e.g., mRNA vaccine) comprises at least one RNA polynucleotide (e.g., mRNA) having one or more open reading frames encoding gH, gL, UL128, UL130, and/or UL131A, or antigenic fragments or epitopes thereof. In some embodiments, the HCMV RNA vaccine (e.g., mRNA vaccine) comprises at least one RNA polynucleotide (e.g., mRNA) having one or more open reading frames encoding gB, gH, gL, UL128, UL130, and/or UL131A, or antigenic fragments or epitopes thereof. In some embodiments, the HCMV RNA vaccine (e.g., mRNA vaccine) comprises at least one RNA polynucleotide (e.g., mRNA) having one or more open reading frames encodes gB, gH, gL, UL128, UL130, and UL131A, or antigenic fragments or epitopes thereof, and further comprises an RNA polynucleotide (e.g., mRNA) having an open reading frame encoding HCMV antigenic polypeptide pp65, or an antigenic fragment or epitope thereof. In some embodiments, the HCMV RNA vaccine (e.g., mRNA vaccine) comprises at least one RNA polynucleotide (e.g., mRNA) having one or more open reading frames encoding HCMV antigenic polypeptide pp65, or antigenic fragments or epitopes thereof. In some embodiments, the pp65 polypeptide sequence contains a deletion of amino acids 435-438. In some embodiments, a first HCMV vaccine and a second HCMV vaccine are administered. A first HCMV vaccine can comprise an RNA polynucleotide having an open reading frame encoding HCMV antigenic polypeptide pp65, or an antigenic fragment or epitope thereof, while a second HCMV vaccine can comprise at least one RNA polynucleotide having one or more open reading frames encoding HCMV antigenic polypeptides gH, gL, UL128, UL130, and/or UL131A, or antigenic fragments or epitopes thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof.

Within HCMV vaccines described herein, the various components can be formulated together or separately. In some embodiments, the RNA polynucleotides (e.g., mRNAs) encoding gB, gH, gL, UL128, UL130, UL131A, or antigenic fragments or epitopes thereof, may be formulated in one HCMV vaccine composition and can be formulated at equal ratios (e.g., at 1:1:1:1:1 ratio) or can be formulated at different ratios. In some embodiments, the RNA polynucleotides (e.g., mRNAs) ending pp65, or antigenic fragments or epitopes thereof, are formulated in a separate HCMV vaccine composition. In other embodiments, the RNA polynucleotides (e.g., mRNAs) encoding gB, gH, gL, UL128, UL130, UL131A, or antigenic fragments or epitopes thereof are formulated together with the RNA polynucleotides (e.g., mRNAs) ending pp65, or antigenic fragments or epitopes thereof.

HCMV vaccines described herein can be administered to donors and/or recipients of organ transplant. Donors and/or recipients can be seronegative or seropositive. In some embodiments, the HCMV mRNA vaccines of the present disclosure are administered to: seronegative recipients receiving a transplant from a seropositive donor; seronegative recipients receiving a transplantation from a seronegative donor; or seropositive recipients receiving a transplant from a seropositive or seronegative donor. The HCMV mRNA vaccines of the present disclosure may also be administered to transplant donors, either seronegative or seropositive, to prevent or treat HCMV.

HCMV mRNA vaccines described herein may be administered to transplant recipients or donors before or after the transplantation. If given before transplantation, it may be given, e.g., 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, or more before the transplantation. If give after the transplantation, it may be given, e.g., 1 day, 2 days, 3 days. 4 days, 5 days, 6 days, 7 days, 8 days, 9 days, 10 days, 2 weeks, 3 weeks, 4 weeks, 5 weeks, 6 weeks, 7 weeks, 8 weeks, 3 months, 4 months, 6 months, or more after the transplantation. The dosage of the HCMV mRNA vaccines may include any of the dosages described herein. Booster doses may also be given after one or two primary doses. In some embodiments, two primary doses are given at 0 and 1 month, and a booster dose is given at 6 months.

In some embodiments, in which two HCMV vaccines are administered, the two HCMV vaccines may be administered simultaneously or sequentially. For example, in some embodiments, a first HCMV vaccine comprising an RNA polynucleotide having an open reading frame encoding HCMV antigenic polypeptide pp65, or an antigenic fragment or epitope thereof is administered before a second HCMV vaccine comprising at least one RNA polynucleotide having one or more open reading frames encoding HCMV antigenic polypeptides gH, gL, UL128, UL130, and/or UL131A, or antigenic fragments or epitopes thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof. For example, the first HCMV vaccine can be administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days before the second vaccine. In some embodiments, the first HCMV vaccine is administered at least 1, 2, 3, or 4 weeks before the second HCMV vaccine. In some embodiments, the first HCMV vaccine is administered at least 1, 2, or 3 weeks before the second HCMV vaccine.

In some embodiments, HCMV mRNA vaccines described herein may be given in combination with other antiviral drugs, e.g., Ganciclovir and derivatives, CMV-CTL, HCMV specific antibodies, Brincidofovir, or Letermovir.

It has been discovered that the mRNA vaccines described herein are superior to current vaccines in several ways. First, the lipid nanoparticle (LNP) delivery is superior to other formulations including liposome or protamine based approachs described in the literature and no additional adjuvants are to be necessary. The use of LNPs enables the effective delivery of chemically modified or unmodified (no nucleotide modifications) mRNA vaccines. Both modified and unmodified LNP formulated mRNA vaccines are superior to conventional vaccines by a significant degree. In some embodiments the mRNA vaccines of the invention are superior to conventional vaccines by a factor of at least 10 fold, 20 fold, 40 fold, 50 fold, 100 fold, 500 fold or 1,000 fold.

Although attempts have been made to produce functional RNA vaccines, including mRNA vaccines and self-replicating RNA vaccines, the therapeutic efficacy of these RNA vaccines have not yet been fully established. Quite surprisingly, the inventors have discovered, according to aspects of the invention a class of formulations for delivering mRNA vaccines in vivo that results in significantly enhanced, and in many respects synergistic, immune responses including enhanced antigen generation and functional antibody production with neutralization capability. These results can be achieved even when significantly lower doses of the mRNA are administered in comparison with mRNA doses used in other classes of lipid based formulations. The formulations of the invention have demonstrated significant unexpected immune responses sufficient to establish the efficacy of functional mRNA vaccines as prophylactic and therapeutic agents. Additionally, self-replicating RNA vaccines rely on viral replication pathways to deliver enough RNA to a cell to produce an immunogenic response. The formulations of the invention do not require viral replication to produce enough protein to result in a strong immune response. Thus, the mRNA of the invention are not self-replicating RNA and do not include components necessary for viral replication.

### Nucleic Acids/Polynucleotides

Human cytomegalovirus (HCMV) vaccines, as provided herein, comprise at least one (one or more) ribonucleic acid (RNA) polynucleotide having an open reading frame encoding at least one HCMV antigenic polypeptide. The term “nucleic acid,” in its broadest sense, includes any compound and/or substance that comprises a polymer of nucleotides. These polymers are referred to as polynucleotides.

In some embodiments, at least one RNA polynucleotide of a HCMV vaccine is encoded by at least one nucleic acid sequence selected from any of SEQ ID NOs: 1-31, 58, 60, 62, 64, 66, 68, 70, and 80-83. In some embodiments, at least one RNA polynucleotide of a HCMV vaccine is encoded by at least one fragment of a nucleic acid sequence selected from any of SEQ ID NOs: 1-31, 58, 60, 62, 64, 66, 68, 70, and 80-83.

In some embodiments, an RNA vaccine comprises an RNA polynucleotide having an open reading frame encoded by SEQ ID NO:58, or an antigenic fragment or epitope thereof; an RNA polynucleotide having an open reading frame encoded by SEQ ID NO:60, or an antigenic fragment or epitope thereof; an RNA polynucleotide having an open reading frame encoded by SEQ ID NO:62, or an antigenic fragment or epitope thereof; an RNA polynucleotide having an open reading frame encoded by SEQ ID NO:64, or an antigenic fragment or epitope thereof: an RNA polynucleotide having an open reading frame encoded by SEQ ID NO:66, or an antigenic fragment or epitope thereof, and an RNA polynucleotide having an open reading frame encoded by SEQ ID NO:68, or an antigenic fragment or epitope thereof. In some embodiments, an RNA vaccine also comprises an RNA polynucleotide having an open reading frame encoded by SEQ ID NO:70, or an antigenic fragment or epitope thereof.

In some embodiments, an RNA vaccine comprises an RNA polynucleotide having an open reading frame encoded by SEQ ID NO:90, or an antigenic fragment or epitope thereof; an RNA polynucleotide having an open reading frame encoded by SEQ ID NO:91, or an antigenic fragment or epitope thereof; an RNA polynucleotide having an open reading frame encoded by SEQ ID NO:144, or an antigenic fragment or epitope thereof; an RNA polynucleotide having an open reading frame encoded by SEQ ID NO:87, or an antigenic fragment or epitope thereof; an RNA polynucleotide having an open reading frame encoded by SEQ ID NO:89, or an antigenic fragment or epitope thereof; an RNA polynucleotide having an open reading frame encoded by SEQ ID NO:86, or an antigenic fragment or epitope thereof; and/or an RNA polynucleotide having an open reading frame encoded by SEQ ID NO:92, or an antigenic fragment or epitope thereof.

It should be appreciated that open reading frame sequences can be combined with multiple different regulatory sequences, such as untranslated regions (UTRs). ORFs described herein can be linked to different UTRs. In some embodiments, a 5′ UTR sequence comprises SEQ ID NO: 146. In some embodiments, a 3′UTR sequence comprises SEQ ID NO:147.

In some embodiments, an RNA vaccine comprises an RNA polynucleotide having an open reading frame encoded by SEQ ID NO:90, or an antigenic fragment or epitope thereof, with a 5′ UTR sequence comprising SEQ ID NO: 146 and/or a 3′UTR sequence comprising SEQ ID NO: 147: an RNA polynucleotide having an open reading frame encoded by SEQ ID NO:91, or an antigenic fragment or epitope thereof, with a 5′ UTR sequence comprising SEQ ID NO: 146 and/or a 3′UTR sequence comprising SEQ ID NO: 147; an RNA polynucleotide having an open reading frame encoded by SEQ ID NO:144, or an antigenic fragment or epitope thereof, with a 5′ UTR sequence comprising SEQ ID NO: 146 and/or a 3′UTR sequence comprising SEQ ID NO: 147; an RNA polynucleotide having an open reading frame encoded by SEQ ID NO:87, or an antigenic fragment or epitope thereof, with a 5′ UTR sequence comprising SEQ ID NO: 146 and/or a 3′UTR sequence comprising SEQ ID NO: 147; an RNA polynucleotide having an open reading frame encoded by SEQ ID NO:89, or an antigenic fragment or epitope thereof, with a 5′ UTR sequence comprising SEQ ID NO: 146 and/or a 3′UTR sequence comprising SEQ ID NO: 147; an RNA polynucleotide having an open reading frame encoded by SEQ ID NO:86, or an antigenic fragment or epitope thereof, with a 5′ UTR sequence comprising SEQ ID NO: 146 and/or a 3′UTR sequence comprising SEQ ID NO: 147; and/or an RNA polynucleotide having an open reading frame encoded by SEQ ID NO:92, or an antigenic fragment or epitope thereof, with a 5′ UTR sequence comprising SEQ ID NO: 146 and/or a 3′UTR sequence comprising SEQ ID NO: 147.

In some embodiments, a transplant donor or recipient is administered an RNA vaccine composition comprising an RNA polynucleotide having an open reading frame encoded by SEQ ID NO:58, or an antigenic fragment or epitope thereof; an RNA polynucleotide having an open reading frame encoded by SEQ ID NO:60, or an antigenic fragment or epitope thereof; an RNA polynucleotide having an open reading frame encoded by SEQ ID NO:62, or an antigenic fragment or epitope thereof; an RNA polynucleotide having an open reading frame encoded by SEQ ID NO:64, or an antigenic fragment or epitope thereof; an RNA polynucleotide having an open reading frame encoded by SEQ ID NO:66, or an antigenic fragment or epitope thereof, and an RNA polynucleotide having an open reading frame encoded by SEQ ID NO:68, or an antigenic fragment or epitope thereof. In some embodiments, the transplant donor or recipient is also administered an RNA polynucleotide having an open reading frame encoded by SEQ ID NO:70, or an antigenic fragment or epitope thereof. The RNA polynucleotide having an open reading frame encoded by SEQ ID NO:70, or an antigenic fragment or epitope thereof can be formulated together or separately with the other RNA polynucleotides administered to the transplant donor or recipient and can be administered either together or separately from the other RNA polynucleotides administered to the transplant donor or recipient.

In some embodiments, a transplant donor or recipient is only administered an RNA polynucleotide having an open reading frame encoded by SEQ ID NO:70, or an antigenic fragment or epitope thereof.

Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. In some preferred embodiments, an mRNA is translated in vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.” One of ordinary skill in the art would understand how to identify an mRNA sequence based on the corresponding DNA sequence.

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

Some embodiments of the present disclosure provide HCMV vaccines that include at least one ribonucleic acid (RNA) polynucleotide having an open reading frame encoding at least one HCMV antigenic polypeptide or an immunogenic fragment or epitope thereof. Some embodiments of the present disclosure provide HCMV vaccines that include at least one RNA polynucleotide having an open reading frame encoding two or more HCMV antigenic polypeptides or an immunogenic fragment or epitope thereof. Some embodiments of the present disclosure provide HCMV vaccines that include two or more RNA polynucleotides having an open reading frame encoding two or more HCMV antigenic polypeptides or immunogenic fragments or epitopes thereof. The one or more HCMV antigenic polypeptides may be encoded on a single RNA polynucleotide or may be encoded individually on multiple (e.g., two or more) RNA polynucleotides.

Some embodiments of the present disclosure provide HCMV vaccines that include at least one ribonucleic acid (RNA) polynucleotide having a single open reading frame encoding two or more (for example, two, three, four, five, or more) HCMV antigenic polypeptides or an immunogenic fragment or epitope thereof. Some embodiments of the present disclosure provide HCMV vaccines that include at least one ribonucleic acid (RNA) polynucleotide having more than one open reading frame, for example, two, three, four, five or more open reading frames encoding two, three, four, five or more HCMV antigenic polypeptides. In either of these embodiments, the at least one RNA polynucleotide may encode two or more HCMV antigenic polypeptides selected from gH, gB, gL, gO, gM, gN, UL83, UL123, UL128, UL130, UL131A, and fragments or epitopes thereof. In some embodiments, the at least one RNA polynucleotide encodes UL83 and UL123. In some embodiments, the at least one RNA polynucleotide encodes gH and gL. In some embodiments, the at least one RNA polynucleotide encodes UL128, UL130, and UL131A. In some embodiments, the at least one RNA polynucleotide encodes gH, gL, UL128, UL130, and UL131A.

In some embodiments, a vaccine comprises an RNA polynucleotide having an open reading frame encoding HCMV antigenic polypeptide gH, or an antigenic fragment or epitope thereof: an RNA polynucleotide having an open reading frame encoding HCMV antigenic polypeptide gL, or an antigenic fragment or epitope thereof; an RNA polynucleotide having an open reading frame encoding HCMV antigenic polypeptide UL128, or an antigenic fragment or epitope thereof; an RNA polynucleotide having an open reading frame encoding HCMV antigenic polypeptide UL130, or an antigenic fragment or epitope thereof; an RNA polynucleotide having an open reading frame encoding HCMV antigenic polypeptide UL131A, or an antigenic fragment or epitope thereof; and an RNA polynucleotide having an open reading frame encoding HCMV antigenic polypeptide gB, or an antigenic fragment or epitope thereof. In some embodiments, the vaccine also comprises an RNA polynucleotide having an open reading frame encoding HCMV antigenic polypeptide pp65, or an antigenic fragment or epitope thereof. In some embodiments, the pp65 polypeptide encoded by the RNA polynucleotide contains a deletion of amino acids 435-438. In some embodiments, the pp65 polypeptide encoded by the RNA polynucleotide comprises SEQ ID NO:71. In some embodiments, the pp65 polypeptide is part of a fusion protein. In some embodiments, pp65 polypeptide, or a fragment thereof, is fused to IE1 or a fragment thereof.

In some embodiments, in which the at least one RNA polynucleotide has a single open reading frame encoding two or more (for example, two, three, four, five, or more) HCMV antigenic polypeptides, the RNA polynucleotide may further comprise additional sequence, for example, a linker sequence or a sequence that aids in the processing of the HCMV RNA transcripts or polypeptides, for example a cleavage site sequence. In some embodiments, the additional sequence may be a protease sequence, such as a furin sequence. Furin, also referred to as PACE (paired basic amino acid cleaving enzyme), is a calcium-dependent serine endoprotease that cleaves precursor proteins into biologically active products at paired basic amino acid processing sites. Some of its substrates include the following: proparathyroid hormone, transforming growth factor beta 1 precursor, proalbumin, pro-beta-secretase, membrane type-1 matrix metalloproteinase, beta subunit of pro-nerve growth factor, and von Willebrand factor. The envelope proteins of certain viruses must be cleaved by furin in order to become fully functional, while some viruses require furin processing during their entry into host cells. T cells require furin to maintain peripheral immune tolerance. In some embodiments, the additional sequence may be self-cleaving 2A peptide, such as a P2A, E2A, F2A, and T2A sequence. In some embodiments, the linker sequences and cleavage site sequences are interspersed between the sequences encoding HCMV polypeptides. 2A peptides are “self-cleaving” small peptides, approximately 18-22 amino acids in length. Ribosomes skip the synthesis of a glycyl-prolyl peptide bond at the C-terminus of a 2A peptide, resulting in the cleavage of the 2A peptide and its immediate downstream peptide. They are frequently used in biomedical research to allow for the simultaneous expression of more than one gene in cells using a single plasmid. There are a number of 2A peptides, including the following: foot-and-mouth disease virus (FMDV) 2A (F2A), equine rhinitis A virus (ERAV) 2A (E2A), porcine teschovirus-1 2A (P2A), and Thoseaasigna virus 2A (T2A). T2A has the highest cleavage efficiency (close to 100%), followed by E2A, P2A, and F2A. Amino acid sequences are the following: P2A:(GSG)ATNFSLLKQAGDVEENPGP (SEQ ID NO: 153); T2A: (GSG)EGRGSLLTCGDVEENPGP (SEQ ID NO: 154); E2A: (GSG)QCTNYALLKLAGDVESNPGP (SEQ ID NO: 155); F2A: (GSG)VKQTLNFDLLKLAGDVESNPGP (SEQ ID NO: 156). In some embodiments, the linker sequences and cleavage site sequences are interspersed between the sequences encoding HCMV polypeptides. In some embodiments, the RNA polynucleotide is encoded by SEQ ID NO: 27, SEQ ID NO: 28, SEQ ID NO: 29, SEQ ID NO: 30 or SEQ ID NO: 31.

In some embodiments, a RNA polynucleotide of a HCMV vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 antigenic polypeptides. In some embodiments, a RNA polynucleotide of a HCMV vaccine encodes at least 10, 20, 30, 40, 50, 60, 70, 80, 90 or 100 antigenic polypeptides. In some embodiments, a RNA polynucleotide of a HCMV vaccine encodes at least 100 or at least 200 antigenic polypeptides. In some embodiments, a RNA polynucleotide of a HCMV vaccine encodes 1-10, 5-15, 10-20, 15-25, 20-30, 25-35, 30-40, 35-45, 40-50, 1-50, 1-100, 2-50 or 2-100 antigenic polypeptides.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 90% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 85% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 80% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 75% sequence identity to a naturally-occurring or wild-type sequence (e.g., a naturally-occurring or wild-type mRNA sequence encoding a polypeptide or protein of interest (e.g., an antigenic protein or polypeptide.

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares between 65% and 75 or about 80% sequence identity to a naturally-occurring or wild-type sequence (e.g., a naturally-occurring or wild-type mRNA sequence encoding a polypeptide or protein of interest (e.g., an antigenic protein or polypeptide.

The skilled artisan will appreciate that, except where otherwise noted, polynucleotide sequences set forth in the instant application will recite “T”s in a representative DNA sequence but where the sequence represents RNA, the “T”s would be substituted for “U”s.

### Antigens/Antigenic Polypeptides

In some embodiments, an antigenic polypeptide is an HCMV glycoprotein. For example, a HCMV glycoprotein may be HCMV gB, gH, gL, gO, gN, or gM or an immunogenic fragment or epitope thereof. In some embodiments, the antigenic polypeptide is a HCMV gH polypeptide. In some embodiments, the antigenic polypeptide is a HCMV gL polypeptide. In some embodiments, the antigenic polypeptide is a HCMV gB polypeptide. In some embodiments, the antigenic polypeptide is a HCMV gO polypeptide. In some embodiments, the antigenic polypeptide is a HCMV gN polypeptide. In some embodiments, the antigenic polypeptide is a HCMV gM polypeptide. In some embodiments, the antigenic polypeptide is a HCMV gC polypeptide. In some embodiments, the antigenic polypeptide is a HCMV gN polypeptide. In some embodiments, the antigenic polypeptide is a HCMV gM polypeptide.

In some embodiments, an antigenic polypeptide is a HCMV protein selected from UL83. UL123, UL128, UL130, and UL131A or an immunogenic fragment or epitope thereof. In some embodiments, the antigenic polypeptide is a HCMV UL83 polypeptide. In some embodiments, the antigenic polypeptide is a HCMV UL123 polypeptide. In some embodiments, the antigenic polypeptide is a HCMV UL128 polypeptide. In some embodiments, the antigenic polypeptide is a HCMV UL130 polypeptide. In some embodiments, the antigenic polypeptide is a HCMV UL131A polypeptide.

In some embodiments, the antigenic HCMV polypeptide comprises two or more HCMV polypeptides. The two or more HCMV polypeptides can be encoded by a single RNA polynucleotide or can be encoded by two or more RNA polynucleotides, for example, each glycoprotein encoded by a separate RNA polynucleotide. In some embodiments, the two or more HCMV polypeptides can be any combination of HCMV gH, gL, gB, gO, gN, gM, UL83, UL123, UL128, UL130, and UL131A polypeptides or immunogenic fragments or epitopes thereof. In some embodiments, the two or more HCMV polypeptides can be any combination of HCMV gH and a polypeptide selected from gL, gB, gO, gN, gM, UL83, UL123. UL128, UL130, and UL131A polypeptides or immunogenic fragments or epitopes thereof. In some embodiments, the two or more HCMV polypeptides can be any combination of HCMV gB and a polypeptide selected from gH, gL, gO, gN, gM, UL83, UL123, UL128. UL130, and UL131A polypeptides or immunogenic fragments or epitopes thereof. In some embodiments, the two or more HCMV polypeptides can be any combination of HCMV gL and a polypeptide selected from gH, gB, gO, gN, gM, UL83, UL123, UL128, UL130, and UL131A polypeptides or immunogenic fragments or epitopes thereof. In some embodiments, the two or more HCMV polypeptides can be any combination of HCMV gH, gL and a polypeptide selected from gB, gO, gN, gM, UL83, UL123, UL128, UL130, and UL131A polypeptides or immunogenic fragments or epitopes thereof. In some embodiments, the two or more HCMV polypeptides can be any combination of HCMV gH, gL, and a glycoprotein selected from gB, gH, gK, gL, gC, gN, and gM polypeptides or immunogenic fragments or epitopes thereof. In some embodiments, the two or more HCMV polypeptides can be any combination of HCMV gH, gL, and a polypeptide selected from UL83, UL123, UL128, UL130, and UL131A polypeptides or immunogenic fragments or epitopes thereof. In some embodiments, the two or more HCMV polypeptides are UL128. UL130, and UL131A. In some embodiments, the two or more HCMV polypeptides are gH and gL. In some embodiments, the two or more HCMV polypeptides are gH, gL, UL128, UL130, and UL131A. In some embodiments, the two or more HCMV polypeptides are gB, gH, gL, UL128, UL130, and UL131A.

HCMV vaccines described herein can further include the HCMV tegument protein pp65. This protein is a target antigen for HCMV-specific cytotoxic T lymphocytes (CTL) responses. (Mclaughlin-Taylor et al., J. Med. Virol. 43:103-110 (1994)).

Pp65 is the major constituent of extracellular virus particles and is the major tegument protein responsible for modulating/evading the host cell immune response during HCMV infections (e.g., in McLaughlin-Taylor et al., J Med Virol 1994, 43: 103-110). Further, pp65 is implicated in counteracting both innate and adaptive immune responses during HCMV infections (e.g., in Kalejta et al., J Gen Virol 2006, 87: 1763-1779). Pp65's role in immune evasion is largely attributable to its targeting of both humoral and cellular immunity as well as serving as the dominant target antigen of cytotoxic T lymphocytes (e.g., in McLaughlin-Taylor et al., J Med Virol 1994, 43: 103-110). Further, pp65 mediates the phosphorylation of viral immediate-early proteins (IE), produced abundantly early after infection, which blocks their presentation to the major histocompatibility complex class I molecules (Gilbert et al., Nature, 383:720-722, 1996). pp65 also plays a role in immune evasion during HCMV infections through the inhibition of natural killer cell cytotoxicity (e.g., in Arnon et al., Nat Immunol 2005, 6: 515-523) and/or attenuation of the interferon response (e.g., in Abate et al., J Virol 2004, 78: 10995-11006). It has also been shown that a pp65-IE1 fusion protein is able to induce both cellular and humoral immune response against HCMV (Reap et al., Clin Vaccine Immunol, vol. 14 no. 6 748-755, 2007; Lilja et al., Vaccine, November 19; 30(49), 2002).

Embodiments of the present disclosure provide RNA (e.g., mRNA) vaccines that include polynucleotides encoding a HCMV structural protein, e.g., pp65, or a pp65-IE1 fusion protein, for eliciting protective immunity against CMV infection. Tables 8 and 9 provide nucleic acid and protein sequences for pp65 and fusion proteins encompassing pp65. In some embodiments, a pp65 RNA polynucleotide is encoded by a sequence within Table 8 Table 9, or Table 13. In some embodiments, a pp65 RNA polynucleotide is encoded by SEQ ID NO:70 or SEQ ID NO: 93. In some embodiments, the RNA polynucleotide encodes a pp65 protein provided in Table 8 or Table 9. In some embodiments, the pp65 protein comprises SEQ ID NO:71. In some embodiments, the pp65 polypeptide is part of a fusion protein. In some embodiments, pp65 polypeptide, or a fragment thereof, is fused to IE1 or a fragment thereof.

The present disclosure includes variant HCMV antigenic polypeptides. In some embodiments, the variant HCMV antigenic polypeptide is a variant pp65 polypeptide. In some embodiments, a variant pp65 polypeptide contains a deletion of amino acids 435-438 relative to the wild type pp65 sequence. The variant pp65 polypeptide can comprise SEQ ID NO:71. A pp65 protein with a deletion of amino acids 435-438 is also referred to herein as “pp65mut” or “pp65^{ΔP}.” In some embodiments, pp65mut is encoded by a polynucleotide comprising the nucleotide sequence of SEQ ID NO: 92. In some embodiments, the variant pp65 polypeptide is part of a fusion protein. In some embodiments, a variant pp65 polypeptide, or a fragment thereof, is fused to IE1 or a fragment thereof.

The use of pp65, including variant forms of pp65, in vaccine compositions is described in and incorporated by reference from: U.S. Pat Nos. 7,387,782, 7,025,969, 6,133,433, 6,207,161, 6,074,645, 6,251,399, 6,727,093, 6,726,910, 6,843,992, 6,544,521, 6,951,651, 8,580,276, 7,163,685, 6,242,567, 6,835,383, 6,156,317, 6,562,345, 8,673,317, 8,278,093, 7,888,112, 9,180,162, 7,410,795, 6,579,970, 7,202,331, 8,029,796, 8,425,898, US 2015-0335732, US 2016-0213771, WO 2015/047901, US 2012-0213818, US 2014-0127216,7,041,442, 8,617,560, 7,976,845, US 2015-0273051, US 2015-0174237, 6,448,389, WO 2015/082570, 7,419,674, US 2014-0308308, and US 2013-0202708, which are incorporated by reference herein in their entireties.

The present disclosure includes variant HCMV antigenic polypeptides. In some embodiments, the variant HCMV antigenic polypeptide is a variant HCMV gH polypeptide. In some embodiments, the variant HCMV antigenic polypeptide is a variant HCMV gL polypeptide. In some embodiments, the variant HCMV antigenic polypeptide is a variant HCMV gB polypeptide. The variant HCMV polypeptides are designed to expedite passage of the antigenic polypeptide through the ER/golgi, leading to increased surface expression of the antigen. In some embodiments, the variant HCMV polypeptides are truncated to delete one or more of the following domains: hydrophobic membrane proximal domain, transmembrane domain, and cytoplasmic domain. In some embodiments, the variant HCMV polypeptides are truncated to include only the ectodomain sequence. For example, the variant HCMV polypeptide can be a truncated HCMV gH polypeptide, truncated HCMV gB polypeptide, or truncated HCMV gL polypeptide comprising at least amino acids 1-124, including, for example, amino acids 1-124, 1-140, 1-160, 1-200, 1-250, 1-300, 1-350, 1-360, 1-400, 1-450, 1-500, 1-511, 1-550, and 1-561, as well as polypeptide fragments having fragment sizes within the recited size ranges.

In some embodiments, a HCMV antigenic polypeptide is longer than 25 amino acids and shorter than 50 amino acids. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain or multichain polypeptides such as antibodies or insulin and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90% identity with a native or reference sequence.

In some embodiments “variant mimics” are provided. As used herein, the term “variant mimic” is one which contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic, for example, phenylalanine may act as an inactivating substitution for tyrosine; or alanine may act as an inactivating substitution for serine.

“Orthologs” refers to genes in different species that evolved from a common ancestral gene by speciation. Normally, orthologs retain the same function in the course of evolution. Identification of orthologs is critical for reliable prediction of gene function in newly sequenced genomes.

“Analogs” is meant to include polypeptide variants which differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini or any combination thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide or polynucleotide based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In some embodiments, the polypeptides further comprise additional sequences or functional domains. For example, the HCMV polypeptides of the present disclosure may comprise one or more linker sequences. In some embodiments, the HCMV of the present invention may comprise a polypeptide tag, such as an affinity tag (chitin binding protein (CBP), maltose binding protein (MBP), glutathione-S-transferase (GST). SBP-tag, Strep-tag, AviTag, Calmodulin-tag); solubilization tag; chromatography tag (polyanionic amino acid tag, such as FLAG-tag); epitope tag (short peptide sequences that bind to high-affinity antibodies, such as V5-tag, Myc-tag, VSV-tag, Xpress tag. E-tag, S-tag, and HA-tag); fluorescence tag (e.g., GFP). In some embodiments, the HCMV of the present invention may comprise an amino acid tag, such as one or more lysines, histidines, or glutamates, which can be added to the polypeptide sequences (e.g., at the N-terminal or C-terminal ends). Lysines can be used to increase peptide solubility or to allow for biotinylation. Protein and amino acid tags are peptide sequences genetically grafted onto a recombinant protein. Sequence tags are attached to proteins for various purposes, such as peptide purification, identification, or localization, for use in various applications including, for example, affinity purification, protein array, western blotting, immunofluorescence, and immunoprecipitation. Such tags are subsequently removable by chemical agents or by enzymatic means, such as by specific proteolysis or intein splicing.

Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

### Multiprotein and Multicomponent Vaccines

The present disclosure encompasses HCMV vaccines, e.g., vaccines against human cytomegalovirus, comprising multiple RNA (e.g., mRNA) polynucleotides, each encoding a single antigenic polypeptide, as well as HCMV vaccines comprising a single RNA polynucleotide encoding more than one antigenic polypeptide (e.g., as a fusion polypeptide). Thus, it should be understood that a vaccine composition comprising a RNA polynucleotide having an open reading frame encoding a first HCMV antigenic polypeptide and a RNA polynucleotide having an open reading frame encoding a second HCMV antigenic polypeptide encompasses (a) vaccines that comprise a first RNA polynucleotide encoding a first HCMV antigenic polypeptide and a second RNA polynucleotide encoding a second HCMV antigenic polypeptide, and (b) vaccines that comprise a single RNA polynucleotide encoding a first and second HCMV antigenic polypeptide (e.g., as a fusion polypeptide). HCMV RNA vaccines of the present disclosure, in some embodiments, comprise 2-10 (e.g., 2, 3, 4, 5, 6, 7, 8, 9 or 10), or more. RNA polynucleotides having an open reading frame, each of which encodes a different HCMV antigenic polypeptide (or a single RNA polynucleotide encoding 2-10, or more, different HCMV antigenic polypeptides). In some embodiments, an HCMV RNA vaccine comprises a RNA polynucleotide having an open reading frame encoding an HCMV glycoprotein. In some embodiments, an HCMV RNA vaccine comprises a RNA polynucleotide having an open reading frame encoding an HCMV glycoprotein B (gB), a RNA polynucleotide having an open reading frame encoding an HCMV glycoprotein M (gM), a RNA polynucleotide having an open reading frame encoding an HCMV glyprotein N (gN), a RNA polynucleotide having an open reading frame encoding an HCMV glycoprotein H (gH), a RNA polynucleotide having an open reading frame encoding an HCMV glycoprotein L (gL), and a RNA polynucleotide having an open reading frame encoding an HCMV glycoprotein O (gO). In some embodiments, an HCMV RNA vaccine comprises a RNA polynucleotide having an open reading frame encoding an HCMV gB protein. In some embodiments, an HCMV RNA vaccine comprises a RNA polynucleotide having an open reading frame encoding an HCMV UL128 protein. In some embodiments, an HCMV RNA vaccine comprises a RNA polynucleotide having an open reading frame encoding an HCMV UL130 protein. In some embodiments, an HCMV RNA vaccine comprises a RNA polynucleotide having an open reading frame encoding an HCMV UL131 protein. In some embodiments, an HCMV RNA vaccine comprises a RNA polynucleotide having an open reading frame encoding an HCMV pp65 protein. In some embodiments, the pp65 protein contains a deletion of amino acids 435-438. In some embodiments, an HCMV RNA vaccine comprises a RNA polynucleotide having an open reading frame encoding an HCMV gM and gN proteins. In some embodiments, an HCMV RNA vaccine comprises a RNA polynucleotide having an open reading frame encoding an HCMV gH, gL, and gO proteins. In some embodiments, an HCMV RNA vaccine comprises a RNA polynucleotide having an open reading frame encoding an HCMV gH, gL, UL128, UL130, and UL131A proteins. In some embodiments, an HCMV RNA vaccine comprises RNA polynucleotides having one or more open reading frames encoding an HCMV UL83, UL128, UL123, UL130, or UL131A protein. In some embodiments, the HCMV RNA vaccine further comprises a RNA polynucleotide having an open reading frame encoding one or more (e.g., 2, 3, 4, 5, 6 or 7) HCMV proteins.

In some embodiments, an HCMV RNA vaccine comprises RNA polynucleotides having one or more open reading frames encoding HCMV gH, gL, UL128, UL130, and UL131A proteins, or fragments thereof, and an HCMV gB protein, or fragment thereof.

In some embodiments, an HCMV RNA vaccine comprises an RNA polynucleotide having an open reading frame encoding a gH protein or a fragment thereof, an RNA polynucleotide having an open reading frame encoding a gL protein or a fragment thereof, an RNA polynucleotide having an open reading frame encoding a UL128 protein or a fragment thereof, an RNA polynucleotide having an open reading frame encoding a UL130 protein or a fragment thereof, an RNA polynucleotide having an open reading frame encoding a UL131A protein or a fragment thereof, and an an RNA polynucleotide having an open reading frame encoding a gB protein, or a fragment thereof. In some embodiments, an HCMV RNA vaccine also comprises an RNA polynucleotide having an open reading frame encoding a pp65 protein or a fragment thereof. In some embodiments, the pp65 polypeptide contains a deletion of amino acids 435-438.

In some embodiments, a RNA polynucleotide encodes an HCMV antigenic polypeptide fused to a signal peptide (e.g., SEQ ID NO: 53 or 54). The signal peptide may be fused at the N-terminus or the C-terminus of the antigenic polypeptide.

### Signal Peptides

In some embodiments, antigenic polypeptides encoded by HCMV nucleic acids comprise a signal peptide. Signal peptides, comprising the N-terminal 15-60 amino acids of proteins, are typically needed for the translocation across the membrane on the secretory pathway and thus universally control the entry of most proteins both in eukaryotes and prokaryotes to the secretory pathway. Signal peptides generally include three regions: an N-terminal region of differing length, which usually comprises positively charged amino acids, a hydrophobic region, and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it. The signal peptide is not responsible for the final destination of the mature protein, however. Secretory proteins devoid of further address tags in their sequence are by default secreted to the external environment. Signal peptides are cleaved from precursor proteins by an endoplasmic reticulum (ER)-resident signal peptidase or they remain uncleaved and function as a membrane anchor. During recent years, a more advanced view of signal peptides has evolved, showing that the functions and immunodorminance of certain signal peptides are much more versatile than previously anticipated.

HCMV vaccines of the present disclosure may comprise, for example, RNA polynucleotides encoding an artificial signal peptide, wherein the signal peptide coding sequence is operably linked to and is in frame with the coding sequence of the HCMV antigenic polypeptide. Thus, HCMV vaccines of the present disclosure, in some embodiments, produce an antigenic polypeptide comprising a HCMV antigenic polypeptide fused to a signal peptide. In some embodiments, a signal peptide is fused to the N-terminus of the HCMV antigenic polypeptide. In some embodiments, a signal peptide is fused to the C-terminus of the HCMV antigenic polypeptide.

In some embodiments, the signal peptide fused to the HCMV antigenic polypeptide is an artificial signal peptide. In some embodiments, an artificial signal peptide fused to the HCMV antigenic polypeptide encoded by the HCMV RNA vaccine is obtained from an immunoglobulin protein, e.g., an IgE signal peptide or an IgG signal peptide. In some embodiments, a signal peptide fused to the HCMV antigenic polypeptide encoded by an HCMV mRNA vaccine is an Ig heavy chain epsilon-1 signal peptide (IgE HC SP) having the sequence of: MDWTWILFLVAAATRVHS (SEQ ID NO: 53). In some embodiments, a signal peptide fused to a HCMV antigenic polypeptide encoded by the HCMV RNA vaccine is an IgGₖ chain V-III region HAH signal peptide (IgGₖ SP) having the sequence of METPAQLLFLLLLWLPDTTG (SEQ ID NO: 54). In some embodiments, a signal peptide fused to the HCMV antigenic polypeptide encoded by an HCMV RNA vaccine has an amino acid sequence set forth in SEQ ID NO: 53 or SEQ ID NO: 54. The examples disclosed herein are not meant to be limiting and any signal peptide that is known in the art to facilitate targeting of a protein to ER for processing and/or targeting of a protein to the cell membrane may be used in accordance with the present disclosure.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide may have a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Non-limiting examples of HCMV antigenic polypeptides fused to signal peptides, which are encoded by the HCMV RNA vaccine of the present disclosure, may be found in Table 2, SEQ ID NOs: 32-52.

A signal peptide is typically cleaved from the nascent polypeptide at the cleavage junction during ER processing. The mature HCMV antigenic polypeptide produce by HCMV RNA vaccine of the present disclosure typically does not comprise a signal peptide.

### Chemical Modifications

HCMV RNA vaccines of the present disclosure comprise, in some embodiments, at least one ribonucleic acid (RNA) polynucleotide having an open reading frame encoding at least one HCMV antigenic polypeptide, or an immunogenic fragment thereof, that comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides) that are useful in the vaccines of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine: N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine: N6, N6 (dimethyl)adenine: N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine: 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine: 8-(alkyl)adenine: 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP: 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP: 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-α-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP: 2-Fluoroadenosine TP; 2-Iodoadenosine TP: 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetyleytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine: 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-α-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP: 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP: 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-uimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N(methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP: 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP: 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2.4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP: 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP: 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-α-thiomethoxyuridine TP; 2′-Deoxy-2-b-aminouridine TP; 2′-Deoxy-2′-b-azidouridine TP; 2′-Deoxy-2′-b-bromouridine TP: 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP: Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl; 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 2′ methyl, 2′amino. 2′azido, 2′fluro-adenine; 2′methyl, 2′amino, 2′azido. 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo )-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; lsocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m¹ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (ψ) and 5-methyl-cytidine (m⁵C). In some embodiments, polynucleotides (e.g., RNA polynucleotides, such as mRNA polynucleotides) comprise 1-methyl-pseudouridine (m¹ψ). In some embodiments, polynucleotides (e.g., RNA polynucleotides, such as mRNA polynucleotides) comprise 1-methyl-pseudouridine (m¹ψ) and 5-methyl-cytidine (m⁵C). In some embodiments, polynucleotides (e.g., RNA polynucleotides, such as mRNA polynucleotides) comprise 2-thiouridine (s²U). In some embodiments, polynucleotides (e.g., RNA polynucleotides, such as mRNA polynucleotides) comprise 2-thiouridine and 5-methyl-cytidine (m¹C). In some embodiments, polynucleotides (e.g., RNA polynucleotides, such as mRNA polynucleotides) comprise methoxy-uridine (mo⁵U). In some embodiments, polynucleotides (e.g., RNA polynucleotides, such as mRNA polynucleotides) comprise 5-methoxy-uridine (mo⁵U) and 5-methyl-cytidine (m⁵C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m⁵C). In some embodiments, polynucleotides (e.g., RNA polynucleotides, such as mRNA polynucleotides) comprise N6-methyl-adenosine (m⁶A). In some embodiments, polynucleotides (e.g., RNA polynucleotides, such as mRNA polynucleotides) comprise N6-methyl-adenosine (m⁶A) and 5-methyl-cytidine (m⁵C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m⁵C), meaning that all cytosine residues in the mRNA sequence are replaced with 5-methyl-cytidine (m⁵C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine, nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G. U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures). In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

Thus, in some embodiments, the RNA (e.g., mRNA) vaccines comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (W), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3W), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, N1-ethyl-pseudouridine 3-(3-amino-3-carboxypropyl)uridine (acp3U), I-methyl-3-(3-amino-3-carboxypropyl)pseudouridine (acp3ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C). N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, a-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, O6-methyl-guanosine, 2′-F-ara-guanosine, and 2′-F-guanosine.

In Vitro Transcription of RNA (e.g., mRNA)

HCMV vaccines of the present disclosure comprise at least one RNA polynucleotide, such as a mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

### Methods of Treatment

Provided herein are compositions (e.g., pharmaceutical compositions), methods, kits and reagents for prevention and/or treatment of HCMV in humans and other mammals. HCMV RNA vaccines can be used as therapeutic or prophylactic agents. They may be used in medicine to prevent and/or treat infectious disease. In exemplary aspects, the HCMV RNA vaccines of the invention are used to provide prophylactic protection from human cytomegalovirus infection and may be particularly useful for prevention and/or treatment of immunocompromised and infant patients to prevent or to reduce the severity and/or duration of the clinical manifestation of the cytomegalovirus infection. In some embodiments, vaccines described herein reduce or prevent congenital transmission of HCMV from mother to child.

### Broad Spectrum Vaccines

HCMV RNA (e.g., mRNA) vaccines can be used as therapeutic or prophylactic agents. It is envisioned that there may be situations where persons are at risk for infection with more than one betacoronovirus, for example, at risk for infection with HCMV. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one HCMV strain, a combination vaccine can be administered that includes RNA encoding at least one antigenic polypeptide of a first HCMV and further includes RNA encoding at least one antigenic polypeptide of a second HCMV. RNAs (mRNAs) can be co-formulated, for example, in a single LNP or can be formulated in separate LNPs destined for co-administration.

A method of eliciting an immune response in a subject against a HCMV is provided in aspects of the invention. The method involves administering to the subject a HCMV RNA vaccine comprising at least one RNA polynucleotide having an open reading frame encoding at least one HCMV antigenic polypeptide or an immunogenic fragment thereof, thereby inducing in the subject an immune response specific to HCMV antigenic polypeptide or an immunogenic fragment thereof, wherein anti-antigenic polypeptide antibody titer in the subject is increased following vaccination relative to anti-antigenic polypeptide antibody titer in a subject vaccinated with a prophylactically effective dose of a traditional vaccine against the HCMV. An “anti-antigenic polypeptide antibody” is a serum antibody the binds specifically to the antigenic polypeptide.

A prophylactically effective dose is a therapeutically effective dose that prevents infection with the virus at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the HCMV.

In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against the HCMV.

In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against the HCMV.

In some embodiments the anti-antigenic polypeptide antibody titer in the subject is increased 3 log following vaccination relative to anti-antigenic polypeptide antibody titer in a subject vaccinated with a prophylactically effective dose of a traditional vaccine against the HCMV.

In some embodiments the anti-antigenic polypeptide antibody titer in the subject is increased 5 log following vaccination relative to anti-antigenic polypeptide antibody titer in a subject vaccinated with a prophylactically effective dose of a traditional vaccine against the HCMV.

In some embodiments the anti-antigenic polypeptide antibody titer in the subject is increased 10 log following vaccination relative to anti-antigenic polypeptide antibody titer in a subject vaccinated with a prophylactically effective dose of a traditional vaccine against the HCMV.

A method of eliciting an immune response in a subject against a HCMV is provided in other aspects of the invention. The method involves administering to the subject a HCMV RNA vaccine comprising at least one RNA polynucleotide having an open reading frame encoding at least one HCMV antigenic polypeptide or an immunogenic fragment thereof, thereby inducing in the subject an immune response specific to HCMV antigenic polypeptide or an immunogenic fragment thereof, wherein the immune response in the subject is equivalent to an immune response in a subject vaccinated with a traditional vaccine against the HCMV at 2 times to 100 times the dosage level relative to the RNA vaccine.

In some embodiments the immune response in the subject is equivalent to an immune response in a subject vaccinated with a traditional vaccine at twice the dosage level relative to the HCMV RNA vaccine.

In some embodiments the immune response in the subject is equivalent to an immune response in a subject vaccinated with a traditional vaccine at three times the dosage level relative to the HCMV RNA vaccine.

In some embodiments the immune response in the subject is equivalent to an immune response in a subject vaccinated with a traditional vaccine at 4 times the dosage level relative to the HCMV RNA vaccine.

In some embodiments the immune response in the subject is equivalent to an immune response in a subject vaccinated with a traditional vaccine at 5 times the dosage level relative to the HCMV RNA vaccine.

In some embodiments the immune response in the subject is equivalent to an immune response in a subject vaccinated with a traditional vaccine at 10 times the dosage level relative to the HCMV RNA vaccine.

In some embodiments the immune response in the subject is equivalent to an immune response in a subject vaccinated with a traditional vaccine at 50 times the dosage level relative to the HCMV RNA vaccine.

In some embodiments the immune response in the subject is equivalent to an immune response in a subject vaccinated with a traditional vaccine at 100 times the dosage level relative to the HCMV RNA vaccine.

In some embodiments the immune response in the subject is equivalent to an immune response in a subject vaccinated with a traditional vaccine at 10 times to 1000 times the dosage level relative to the HCMV RNA vaccine.

In some embodiments the immune response in the subject is equivalent to an immune response in a subject vaccinated with a traditional vaccine at 100 times to 1000 times the dosage level relative to the HCMV RNA vaccine.

In other embodiments the immune response is assessed by determining anti-antigenic polypeptide antibody titer in the subject.

In other aspects the invention is a method of eliciting an immune response in a subject against a HCMV by administering to the subject a HCMV RNA vaccine comprising at least one RNA polynucleotide having an open reading frame encoding at least one HCMV antigenic polypeptide or an immunogenic fragment thereof, thereby inducing in the subject an immune response specific to HCMV antigenic polypeptide or an immunogenic fragment thereof, wherein the immune response in the subject is induced 2 days to 10 weeks earlier relative to an immune response induced in a subject vaccinated with a prophylactically effective dose of a traditional vaccine against the HCMV. In some embodiments the immune response in the subject is induced in a subject vaccinated with a prophylactically effective dose of a traditional vaccine at 2 times to 100 times the dosage level relative to the RNA vaccine.

In some embodiments the immune response in the subject is induced 2 days earlier relative to an immune response induced in a subject vaccinated with a prophylactically effective dose of a traditional vaccine.

In some embodiments the immune response in the subject is induced 3 days earlier relative to an immune response induced in a subject vaccinated a prophylactically effective dose of a traditional vaccine.

In some embodiments the immune response in the subject is induced 1 week earlier relative to an immune response induced in a subject vaccinated with a prophylactically effective dose of a traditional vaccine.

In some embodiments the immune response in the subject is induced 2 weeks earlier relative to an immune response induced in a subject vaccinated with a prophylactically effective dose of a traditional vaccine.

In some embodiments the immune response in the subject is induced 3 weeks earlier relative to an immune response induced in a subject vaccinated with a prophylactically effective dose of a traditional vaccine.

In some embodiments the immune response in the subject is induced 5 weeks earlier relative to an immune response induced in a subject vaccinated with a prophylactically effective dose of a traditional vaccine.

In some embodiments the immune response in the subject is induced 10 weeks earlier relative to an immune response induced in a subject vaccinated with a prophylactically effective dose of a traditional vaccine.

A method of eliciting an immune response in a subject against a HCMV by administering to the subject a HCMV RNA vaccine having an open reading frame encoding a first antigenic polypeptide, wherein the RNA polynucleotide does not include a stabilization element, and wherein an adjuvant is not coformulated or co-administered with the vaccine is also provided herein.

### Standard of Care for CMV Prevention and Treatment

A variety of approaches to preventing and/or treating CMV, including immunization strategies, have previously been pursued or are currently being pursued, some of which are summarized below. However, all of these approaches have drawbacks and limitations. (Schleiss et al. (2008). Curr Top Microbiol Immunol. 325:361-382).

### Ganciclovir and Valganciclovir

In some embodiments, Ganciclovir or Valganciclovir is the standard of care therapy for treatment or prevention of CMV infections (Reusser P. et al. (2000); 130(4):101-12; Biron et al. (2006) Antiviral Research 71:154-163).

Ganciclovir (marketed as CYTOVENE® and ZIRGAN®) and Valganciclovir (a prodrug form of Ganciclovir marketed as VALCYTE®) are antiviral medications developed by Hoffmann-La Roche to treat CMV infection. They are analogues of 2′-deoxy-guanosine, which competitively inhibits dGTP incorporation into DNA and, in turn, viral replication (Sugawara M et al., J Pharm Sci. 2000; 89(6):781-9). CYTOVENE-IV (ganciclovir sodium for injection) is FDA approved “for use only in the treatment of cytomegalovirus (CMV) retinitis in immunocompromised patients and for the prevention of CMV disease in transplant patients at risk for CMV disease.” (FDA Label, Jan. 31, 2006, page 1.)

The recommended dose regimen for CYTOVENE-IV for treatment of CMV retinitis for patients with normal renal function includes an induction phase of 5 mg/kg (administered intravenously over an hour) every 12 hours for 14-21 days, followed by a maintenance phase of 5 mg/kg (administered intravenously over an hour) once daily seven days a week or 6 mg/kg once daily five days a week. (Id., page 22.) For prevention of CMV in transplant patients with normal renal function, the recommended dose regimen includes 5 mg/kg (administered intravenously over an hour) every 12 hours for 7-14 days; then 5 mg/kg once daily seven days a week or 6 mg/kg once daily five days a week. (Id.)

In a study involving heart transplant patients, at 120 days post-transplant, the incidence of CMV in seropositive subjects was 9% in subjects receiving treatment compared to 46% in subjects receiving a placebo. (Biron et al. (2006) Antiviral Research 71:154-163, page 157.) in a study involving bone marrow transplant subjects, at 100 days post-transplant the incidence of CMV in treated subjects was 3% compared to 43% in subjects treated with a placebo. (Id.)

One form of Ganciclovir that is marketed by Bausch and Lomb, ZIRGAN®, is in the form of an ophthalmic gel, which is FDA approved for treatment of acute herpetic keratitis (dendritic ulcers.) (FDA label, Sep. 15, 2009, page 4; Wilhelmus K R et al., 2010. Cochrane Database Syst Rev 12: CD002898).

VALCYTE® (valganciclovir hydrochloride) in tablet form is FDA approved in adult patients for treatment of CMV retinitis in patients with acquired immunodeficiency syndrome (AIDS) and prevention of CMV disease in kidney, heart, and kidney-pancreas transplant patients at high risk. (FDA label, Apr. 23, 2015, page 1.) The dose regimen for VALCYTE® is shown in the following table, as depicted on the FDA label dated Apr. 23, 2015:

TABLE 1
Dose regimen for VALCYTE ®
DOSAGE AND ADMINISTRATION
Adult Dosage (2.2)

| | |
|---|---|
| Treatment of CMV | Induction: 900 mg (two 450 mg tablets) twice a |
| retinitis | day for 21 days |
| | Maintenance: 900 mg (two 450 mg tablets) once |
| | a day |
| Prevention of CMV | 900 mg (two 450 mg tablets) once a day within 10 |
| disease in heart or | days of transplantation until 100 days |
| kidney-pancreas | post-transplantation |
| transplant patients | |
| Prevention of CMV | 900 mg (two 450 mg tablets) once a day within 10 |
| disease in kidney | days of tansplantation until 200 days |
| transplant patients | post-transplantation |

Pediatric Dosage (2.3)

| | |
|---|---|
| Prevention of CMV | Dose once a day within 10 days of transplantation |
| disease in kidney | until 200 days post-transplantation according to |
| transplant patients 4 | dosage algorithm (note the calculation of |
| months to 16 years | creatinine clearance using a modified Schwartz |
| of age | formula in children) |
| Prevention of CMV | Dose once a day within 10 days of transplantation |
| disease in heart | until 100 days post-transplantation according to |
| transplant patients 1 | dosage algorithm (note the calculation of |
| month to 16 years | creatinine clearance using a modified Schwartz |
| of age | formula in children) |

An oral form of Ganciclovir was found to have low bioavailability. (Biron et al. (2006) Antiviral Research 71:154-163.) Valganciclovir was reported to have better bioavailability than Ganciclovir. (Pescovitz M D et al., Antimicrob Agents Chemother. 2000; 44(10):2811-5; Biron et al. (2006) Antiviral Research 71:154-163.)

Adverse side effects associated with Ganciclovir and Valganciclovir include: fever, rash, diarrhea, and hematologic effects (such as neutropenia, anemia, and thrombocytopenia), as well as potential reproductive toxicity. Ganciclovir was also found to affect fertility and to be carcinogenic and teratogenic in animal studies. (Biron et al. (2006) Antiviral Research 71:154-163.)

Phase 3 clinical trials involving treatment of CMV infection with Ganciclovir or Valganciclovir include trials associated with clinicaltrials.gov identifier numbers: NCT00000143, NCT00000136, NCT00000134, NCT00497796, NCT00227370, NCT00466817, and NCT00294515. Results of clinical trials involving Ganciclovir or Valganciclovir are summarized in Biron et al. (2006) Antiviral Research 71:154-163, incorporated by reference herein in its entirety.

### Experimental Vaccines in Development for CMV

TransVax™ (also known as ASP0113 and VCL-CB01)

TransVax™ is a CMV vaccine being developed by Vical Incorporated and Astellas Pharma Inc. (Smith et al. (2013) Vaccines 1(4):398-414.) TransVax™ is a bivalent DNA vaccine containing plasmids encoding CMV pp65 and gB antigens formulated in CRL1005 poloxamer and benzalkonium. (Id.; Kharfan-Dabaja et al. (2012) Lancet Infect Dis 12:290-99). The pp65 antigen induces cytotoxic T cell response, conferring cellular immunity, while the gB antigen elicits both cellular immunity and antigen-specific antibody production. Accordingly, the vaccine is intended to induce both cellular and humoral immune responses. The pp65 and gB sequences are modified from wild type protein sequences through deletions and codon optimization, as described on pages 402-403 of Smith et al. (2013) Vaccines 1(4):398-414, incorporated by reference herein in its entirety.

TransVax™ has received orphan drug designation in the United States and Europe for hematopoietic stem cell transplantation (HSCT), e.g., bone marrow transplantation, and solid organ transplantation (SOT) patients.

In a Phase 1 clinical trial, 37.5% and 50% of CMV-subjects, who were dosed with 1 mg and 5 mg, respectively, of the vaccine, demonstrated antibody or T-cell responses. (Page 406 of Smith et al. (2013) Vaccines 1(4):398-414.) A Phase 2 clinical trial was conducted in patients undergoing allogenic haemopoietic stem cell transplantation (ClinicalTrials.gov identifier number NCT00285259) (Kharfan-Dabaja et al. (2012) Lancet Infect Dis 12:290-99). Transplant patients received the experimental vaccine four times, including once before the transplantation. (Id., page 292.) The dose before transplantation was administered between 3-5 days before transplantation, while the doses after transplantation were administered between 21-42 days after transplantation, and at 84 and 196 days after transplantation. (Id.) Endpoints included assessment of safety and reduction in cytomegalovirus viraemia. (Id.) The incidence of cytomegalovirus viraemia was found to be lower in patients who received the vaccine compared to placebo (32.5% (vaccine group) compared to 61.8% (placebo); Table 2, on page 294 of Kharfan-Dabaja et al.). The vaccine was also reported to be well-tolerated and safe. (Id., page 295.) However, after vaccine treatment, rates of viraemia necessitation anti-viral treatment resembled those of placebo controls. (Id., page 296.)

TransVax™ is currently being tested in a Phase 3 clinical trial for treatment of hematopoietic cell transplant (HCT) patients, accorded ClinicalTrials.gov identifier number NCT01877655. The endpoint for the trial is mortality and end organ disease (EOD) 1 year after transplant. The estimated enrollment is 500 and the vaccine is administered by intramuscular injection. TransVax™ is also currently being tested in a Phase 2 clinical trial in CMV-Seronegative kidney transplant recipients receiving an organ from a CMV-Seropositive donor, accorded ClinicalTrials.gov identifier number NCT01974206. The primary outcome being measured in this trial is incidence of CMV viremia one year after first administration of the drug. The enrollment is 150 and the vaccine is administered by intramuscular injection. Subjects included in the trial also received ganciclovir or valganciclovir from within ten days up transplant through randomization.

Clinical trials involving TransVax™ are found at the ClinicalTrials.gov website with the following ClinicalTrials.gov identifier numbers; NCT02103426, NCT01877655, NCT01974206, and NCT01903928.

US patents and published applications that are assigned to Vical Inc. and relate to CMV include: U.S. Pat. Nos. 8,673,317, 9,180,162, 8,278,093, 7,888,112, 7,410,795, which are incorporated by reference herein in their entireties.

### Experimental Vaccines in Development by City of Hope/National Cancer Institute/Helocyte

Several experimental CMV vaccines are being developed by City of Hope and its licensee Helocyte. US patents and published applications that are assigned to City of Hope and relate to CMV include: U.S. Pat. Nos. 7,387,782, 7,025,969, 6,133,433, 6,207,161. U.S. Pat. Nos. 6,074,645, 6,251,399, 6,727,093, 6,726,910, 6,843,992, 6,544,521, 6,951,651, 8,580,276, 7,163,685, 6,242,567, 6,835,383, 6,156,317, 6,562,345, US 2014-0065181 and US 2015-0216965, which are incorporated by reference herein in their entireties.

### i) CMVPepVax

CMVPepVax is an experimental vaccine being developed by City of Hope Medical Center, National Cancer Institute, and Helocyte, Inc. The vaccine includes a pp65 T-cell epitope and a tetanus T-helper epitope in the form of a chimeric peptide, and also includes the adjuvant PF03512676. (Nakamura R et al., Lancet Heamatology (2016) February; 3(2): e87-98).

CMVPepVax was tested in a Phase 1b clinical trial on CMV-seropositive patients who were undergoing haemopoietic stem-cell transplantation (HCT). (Id.) The vaccine was administered on days 28 and 56 through subcutaneous administration. (Id.) It was reported that patients receiving the vaccine showed improved relapse-free survival. (Id.) This clinical trial was accorded ClinicalTrials.gov identifier number NCT01588015. CMVPepVax is currently being tested in a Phase 2 clinical trial to measure efficacy in reducing the frequency of Cytomegalovirus events in patients with hematologic malignancies undergoing donor stem cell transplant, accorded ClinicalTrials.gov identifier number NCT02396134.

### ii) CMV-MVA Triplex

CMV-MVA-Triplex is an experimental CMV vaccine being developed by City of Hope Medical Center, National Cancer Institute, and Helocyte, Inc. (formerly DiaVax Biosciences). This vaccine consists of an inactivated Modified Vaccinia Ankara (MVA) viral vector that encodes the CMV antigens UL83 (pp65), UL123 (IE1) and UL122 (1E2). (NC1 Drug Dictionary.)

CMV-MVA Triplex is currently being tested in a Phase 2 clinical trial investigating efficacy in reducing CMV complications in patients previously infected with CMV and undergoing donor hematopoietic cell transplant. This trial has been accorded ClinicalTrials.gov identifier number NCT02506933. A Phase 1 clinical trial in healthy volunteers with or without previous exposure to CMV is also ongoing (ClinicalTrials.gov identifier No. NCT01941056).

iii) Pentamer

City of Hope and Helocyte, Inc. are also pursuing a pentameric vaccine using a Modified Vaccinia Ankara (MVA) viral vector that encodes the five CMV pentameric subunits. This vaccine is still in preclinical development. (Wussow et al. (2014) PLoS Pathog 10(11): e1004524. doi: 10.1371/journal.ppat.1004524).

gB/MF59

This experimental vaccine, originally developed in the 1990s combines the gB antigen with the MF59 adjuvant. (Pass et al. (2009) J Clin Virol 46(Suppl 4):S73-S76.) Several clinical trials that were conducted in the 1990s, sponsored by Chiron Corporation, indicated that the vaccine was safe. (Id., page 2.) Sanofi Pasteur later obtained the rights to this vaccine. (Id.)

A Phase 2 clinical trial was conducted in postpartum females starting in 1999 (with enrollment completed in 2006) using the endpoint of time to CMV infection. (Id., page 3.) Subjects were administered the vaccine at 0, 1, and 6 months. (Rieder et al. (2014) Clin Microbiol Infect 20 (Suppl. 5):95-102, page 98). Infection with CMV was diagnosed in 8% of vaccine-treated subjects compared to 14% of placebo-treated subjects, respectively (corresponding to 43% efficacy). Results indicated a 50% reduction in rate of CMV infection in subjects treated with the vaccine (3.3% in test subjects compared to 6.6% in placebo-treated subjects). (Id.; Pass et al. (2009) J Clin Virol 46(Suppl 4):S73-S76., page 4.). The 50% reduction in rate of CMV infection has been described as “lower than wished for from a clinical perspective.” (Rieder et al. (2014) Clin Microbiol Infect 20 (Suppl. 5):95-102, page 98.)

A Phase 2 clinical trial has also been conducted with gB/MF59 in kidney and liver transplant patients. (Id., page 100.) It was reported that “high gB-antibody titres correlated with shorter duration of viraemia” and that “duration of viraemia and number of days of ganciclovir treatment were reduced.” (Id.)

Clinical trials involving gB/MF59 are found at the ClinicalTrials.gov website with the following ClinicalTrials.gov identifier numbers: NCT00133497, NCT00815165, and NCT00125502.

US 2009-0104227, assigned to Sanofi Pasteur SA, is incorporated by reference herein in its entirety.

gB/AS01

GlaxoSmithKline is developing an experimental vaccine that includes the gB antigen combined with the AS01 adjuvant. (McVoy (2013) Clinical Infectious Diseases 57(S4):S196-9, page S197.) This vaccine is referred to as GSK1492903A. Clinical trials involving GSK1492903A are found at the ClinicalTrials.gov website with the following ClinicalTrials.gov identifier numbers: NCT00435396 and NCT01357915.

WO 2016/067239 and WO 2015/181142, filed by GlaxoSmithKline Biologicals SA, are incorporated by reference herein in their entireties.

### Towne Vaccine

The CMV Towne vaccine is a live attenuated vaccine. (McVoy (2013) Clinical Infectious Diseases 57(S4):S196-9, page S197.) This vaccine was not successful in protecting against primary maternal infection, at least when administered at a low dose. (Id.) In a trial involving kidney transplant subjects, treatment with this vaccine resulted in reduction of severe disease, while only having a minimal impact on mild disease. (Plotkin et al. (1994) Transplantation 58(11):1176-8.)

Live attenuated vaccines in which sections of the Towne genome have been replaced with sequence from other “low-passage” strains have also been developed, referred to as “Towne-Toledo chimeras,” which were found to be well-tolerated in a Phase 1 clinical trial. (McVoy (2013) Clinical Infectious Diseases 57(S4):S196-9, page S197; Heineman et al. (2006) The Journal of Infectious Diseases 193:1350-60.) Chimeric viral genomes including portions of the Towne genome are described in and incorporated by reference from U.S. Pat. No. 7,204,990, incorporated by reference herein in its entirety.

Another approach that is being explored involves co-administering the Towne vaccine with the adjuvant recombinant interleukin-12 (rhIL-12) (Jacobson et al. (2006) Vaccine 24:5311-9.)

### CMV-CTL

CMV Targeted T-Cell Program (CMV-CTL) represents a cellular immunotherapy approach being developed by Atara Biotherapeutics.

A Phase 1 clinical trial used CMV pp65 or pp65/IE1 peptide mixes to pulse monocytes to expand CMV CTL and investigated the immunologic effects. (Bao et al. (2012) J Immunother 35(3):293-298). CMV specific immune responses were observed in approximately 70% of subjects receiving CTL. (Id., page 5.)

A Phase 2 clinical trial is currently ongoing, investigating third party donor derived CMVpp65 specific T-cells for the treatment of CMV infection or persistent CMV viremia after allogeneic hematopoietic stem cell transplantation. This trial was assigned ClinicalTrials.gov identifier number NCT02136797. A second Phase 2 clinical trial is also ongoing, investigating primary transplant donor derived CMVpp65 specific T-cells for the treatment of CMV infection or persistent CMV viremia after allogeneic hematopoietic stem cell transplantation. This trial was assigned ClinicalTrials.gov identifier number NCT01646645.

### Monoclonal Abs

### Novartis

CSJ148, being developed by Novartis, represents a combination of two monoclonal antibodies that target gB and the CMV pentameric complex. (Dole et al. (2016) Antimicrob Agents Chemother. April 22; 60(5):2881-7). The two antibodies are known as LJP538 and LJP539. (Id.) LJP538, LJP539, and CSJ148 were found to be safe when administered intravenously to healthy volunteers and revealed expected pharmacokinetics for IgG. (Id.) CSJ148 is currently in a Phase 2 clinical trial investigating efficacy and safety in stem cell transplant patients (ClinicalTrials.gov identifier number NCT02268526).

### Theraclone

TCN-202 is a fully human monoclonal antibody being developed by Theraclone for treatment of CMV infection. TCN-202 was found to be safe and well-tolerated in a Phase 1 clinical trial (ClinicalTrial.gov identifier number NCT01594437). A Phase 2 study was initiated in 2013 to investigate efficacy in kidney transplant recipients. (Theraclone Press Release, Sep. 10, 2013.)

### Brincidofovir

Brincidofovir (CMX001) is an experimental lipid-nucleotide conjugate being developed by Chimerix, Durham, N.C., for treatment of DNA viruses including CMV. Brincidofovir received Fast Track designation from the FDA for CMV.

Results from a Phase 3 clinical trial (called “SUPPRESS”) investigating prevention of CMV in subjects undergoing hematopoietic cell transplantation (HCT) were announced in February, 2016. (Chimerix Press Release. Feb. 20, 2016.) It was reported that the trial failed to meet its primary endpoint of preventing CMV at week 24, although an anti-viral effect was observed during the treatment phase. (Id.) The trial involved 452 subjects undergoing HCT who were administered Brincidofovir twice a week for up to fourteen weeks. (Id.) It was speculated that increased use of immunosteroids, such as corticosteroids, for treatment of graft versus host disease (GVHD), after treatment with Brincidofovir, may have contributed to failure to reach the primary endpoint of the trial. (Id.) Other Phase 3 trials were terminated based on the results of the SUPPRESS trial, but Chimerix has indicated that they intend to pursue further Phase 2 trials in subjects undergoing kidney transplants. (Id.)

Information about clinical trials associated with Brincidofovir are found at the ClinicalTrials.gov website, including identifier numbers: NCT02087306, NCT02271347, NCT02167685, NCT02596997, NCT02439970, NCT00793598, NCT01769170, NCT00780182, NCT01241344, NCT00942305, NCT02420080, NCT02439957, NCT01143181, and NCT01610765.

### V160

V160 is an experimental CMV vaccine being developed by Merck, which is based on the attenuated AD169 strain. V160 is currently being tested in a Phase 1 clinical trial evaluating a three dose regimen testing several formulations in healthy adults. This trial was assigned the ClinicalTrials.gov identifier number NCT01986010.

Merck is also pursuing vaccines that target the CMV pentameric complex. (Loughney et al. (2015) jbc.M115.652230.) US patents and published applications assigned to Merck Sharp & Dohme Corp include: US 2014-0220062 and US 2015-0307850, which are incorporated by reference herein in their entireties.

### Letermovir

Letermovir (AIC246) is an antiviral drug being developed by Merck for the treatment of CMV infections (Chemaly et al. (2014) New England Journal of Medicine, 370; 19, May 8, 2014. Verghese et al. (2013) Drugs Future. May; 38(5): 291-298). It was tested in a Phase IIb clinical trial investigating prevention of CMV in HSCT recipients, corresponding to ClinicalTrials.gov identifier number NCT01063829, and was found to reduce the incidence of CMV infection in transplant subjects.

### Redvax GmbH/Pfizer

A preclinical candidate targeting CMV was developed by Redvax GmbH, which spun out from Redbiotec AG. This candidate is now being pursued by Pfizer Inc.

Patents and patent publications assigned to Redvax GmbH or Pfizer and related to CMV include: US 2015-0322115, WO 2015/170287, US 2015-0359879, and WO 2014/068001, incorporated by reference herein in their entireties.

### Therapeutic and Prophylactic Compositions

Provided herein are compositions (e.g., pharmaceutical compositions), methods, kits and reagents for prevention, treatment or diagnosis of HCMV in humans. HCMV RNA vaccines can be used as therapeutic or prophylactic agents. They may be used in medicine to prevent and/or treat infectious disease. In some embodiments, the HCMV vaccines of the invention can be envisioned for use in the priming of immune effector cells, for example, to activate peripheral blood mononuclear cells (PBMCs) ex vivo, which are then infused (re-infused) into a subject.

In exemplary embodiments, one or more HCMV vaccine containing RNA polynucleotides as described herein can be administered to a subject (e.g., a mammalian subject, such as a human subject), and the RNA polynucleotides are translated in vivo to produce an antigenic polypeptide. In some embodiments, the subject is an organ donor or an organ recipient. For example, the subject can be an immunocompromised organ transplant recipient. In some embodiments, the transplant recipient is a hematopoietic cell transplant recipient or a solid organ transplant recipient. In some embodiments, the subject is a woman of child-bearing age. In some embodiments, vaccines described herein reduce or prevent congenital transmission of HCMV from a mother to a child. (Pass et al. (2014) J Ped Infect Dis 3 (suppl 1): S2-S6.)

The HCMV RNA vaccines may be induced for translation of a polypeptide (e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing a HCMV RNA vaccine that contains a polynucleotide that has at least one a translatable region encoding an antigenic polypeptide.

An “effective amount” of one or more HCMV RNA vaccines is provided based, at least in part, on the target tissue, target cell type, means of administration, physical characteristics of the polynucleotide (e.g., size, and extent of modified nucleosides) and other components of the HCMV RNA vaccine, and other determinants. In general, an effective amount of one or more HCMV RNA vaccine compositions provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation from the polynucleotide, decreased nucleic acid degradation (as demonstrated, for example, by increased duration of protein translation from a modified polynucleotide), or altered antigen specific immune response of the host cell.

In some embodiments, RNA vaccines (including polynucleotides their encoded polypeptides) in accordance with the present disclosure may be used for treatment of HCMV.

HCMV RNA vaccines may be administered prophylactically or therapeutically as part of an active immunization scheme to healthy individuals or early in infection during the incubation phase or during active infection after onset of symptoms. In some embodiments, the amount of RNA vaccines of the present disclosure provided to a cell, a tissue or a subject may be an amount effective for immune prophylaxis.

HCMV RNA vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, HCMV RNA vaccines may be administered intramuscularly or intradermally, similarly to the administration of inactivated vaccines known in the art.

The HCMV RNA vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers and produce responses early than commercially available anti-virals.

Provided herein are pharmaceutical compositions including HCMV RNA vaccines and RNA vaccine compositions and/or complexes optionally in combination with one or more pharmaceutically acceptable excipients.

HCMV RNA vaccines may be formulated or administered alone or in conjunction with one or more other components. For instance, HCMV RNA vaccines (vaccine compositions) may comprise other components including, but not limited to, adjuvants. In some embodiments, HCMV RNA vaccines do not include an adjuvant (they are adjuvant free).

HCMV RNA vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, HCMV RNA vaccines are administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigenic polypeptides.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

HCMV RNA vaccines can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with HCMV RNA vaccines (e.g., for transplantation into a subject), hyaluronidase, nanoparticle mimics and combinations thereof.

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the RNA vaccine may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA vaccine does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. Ideally, the inventive nucleic acid does not include an intron.

In some embodiments, the RNA vaccine may or may not contain a enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA vaccine may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Nanoparticle Formulations

In some embodiments, HCMV RNA vaccines are formulated in a nanoparticle. In some embodiments, HCMV RNA vaccines are formulated in a lipid nanoparticle. In some embodiments, HCMV RNA vaccines are formulated in a lipid-polycation complex, referred to as a lipid nanoparticle. The formation of the lipid nanoparticle may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In some embodiments, HCMV RNA vaccines are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the ionizable lipid component, the degree of ionizable lipid saturation, the nature of the PEGylation, ratio of all components and biophysical parameters such as size. In one example by Semple et al. (Nature Biotech. 2010 28:172-176; herein incorporated by reference in its entirety), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200; herein incorporated by reference in its entirety).

In some embodiments, lipid nanoparticle formulations may comprise 35 to 45% ionizable cationic lipid, 40% to 50% ionizable cationic lipid, 50% to 60% ionizable cationic lipid and/or 55% to 65% ionizable cationic lipid. In some embodiments, the ratio of lipid to RNA (e.g., mRNA) in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, a HCMV RNA vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% ionizable cationic lipid: 5-25% neutral lipid: 25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35 to 65%, 45 to 65%, 60%, 57.5%, 50% or 40% on a molar basis.

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation. DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in its entirety).

In some embodiments, lipid nanoparticle formulations include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, lipid nanoparticle formulations include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, lipid nanoparticle formulations include 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.5% of the neutral lipid, 31% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 38.5% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 35% of the sterol, 4.5% or 5% of the PEG or PEG-modified lipid, and 0.5% of the targeting lipid on a molar basis.

In some embodiments, lipid nanoparticle formulations include 40% of a cationic lipid selected from 2,2-dilinoleyl-4-dimethylaminoethyl-11,31-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 15% of the neutral lipid, 40% of the sterol, and 5% of the PEG or PEG-modified lipid on a molar basis.

In some embodiments, lipid nanoparticle formulations include 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.1% of the neutral lipid, 34.3% of the sterol, and 1.4% of the PEG or PEG-modified lipid on a molar basis.

In some embodiments, lipid nanoparticle formulations include 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in its entirety), 7.5% of the neutral lipid, 31.5% of the sterol, and 3.5% of the PEG or PEG-modified lipid on a molar basis.

In some embodiments, lipid nanoparticle formulations consists essentially of a lipid mixture in molar ratios of 20-70% ionizable cationic lipid: 5-45% neutral lipid: 20-55% cholesterol: 0.5-15% PEG-modified lipid. In some embodiments, lipid nanoparticle formulations consists essentially of a lipid mixture in a molar ratio of 20-60% ionizable cationic lipid: 5-25% neutral lipid: 25-55% cholesterol: 0.5-15% PEG-modified lipid.

In some embodiments, the molar lipid ratio is 50/10/38.5/1.5 (mol % ionizable cationic lipid/neutral lipid, e.g., DSPC/Chol/PEG-modified lipid, e.g., PEG-DMG, PEG-DSG or PEG-DPG), 57.2/7.1134.3/1.4 (mol % ionizable cationic lipid/neutral lipid, e.g., DPPC/Chol/PEG-modified lipid, e.g., PEG-cDMA), 40/15/40/5 (mol % ionizable cationic lipid/neutral lipid, e.g., DSPC/Chol/PEG-modified lipid, e.g., PEG-DMG), 50/10/35/4.5/0.5 (mol % ionizable cationic lipid/neutral lipid, e.g., DSPC/Chol/PEG-modified lipid, e.g., PEG-DSG), 50/10/35/5 (ionizable cationic lipid/neutral lipid, e.g., DSPC/Chol/PEG-modified lipid, e.g., PEG-DMG), 40/10/40/10 (mol % ionizable cationic lipid/neutral lipid, e.g., DSPC/Chol/PEG-modified lipid, e.g., PEG-DMG or PEG-cDMA), 35/15/40/10 (mol % ionizable cationic lipid/neutral lipid, e.g., DSPC/Chol/PEG-modified lipid, e.g., PEG-DMG or PEG-cDMA) or 52/13/30/5 (mol % ionizable cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176: Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, lipid nanoparticle formulations may comprise a ionizable cationic lipid, a PEG lipid and a structural lipid and optionally comprise a non-cationic lipid. As a non-limiting example, a lipid nanoparticle may comprise 40-60% of ionizable cationic lipid, 5-15% of a non-cationic lipid, 1-2% of a PEG lipid and 30-50% of a structural lipid. As another non-limiting example, the lipid nanoparticle may comprise 50% ionizable cationic lipid, 10% non-cationic lipid, 1.5% PEG lipid and 38.5% structural lipid. As yet another non-limiting example, a lipid nanoparticle may comprise 55% ionizable cationic lipid, 10% non-cationic lipid, 2.5% PEG lipid and 32.5% structural lipid. In some embodiments, the ionizable cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a ionizable cationic lipid, a non-cationic lipid, a PEG lipid and a structural lipid. As a non-limiting example, the lipid nanoparticle may comprise 40-60% of ionizable cationic lipid, 5-15% of a non-cationic lipid, 1-2% of a PEG lipid and 30-50% of a structural lipid. As another non-limiting example, the lipid nanoparticle may comprise 50% ionizable cationic lipid, 10% non-cationic lipid, 1.5% PEG lipid and 38.5% structural lipid. As yet another non-limiting example, the lipid nanoparticle may comprise 55% ionizable cationic lipid, 10% non-cationic lipid, 2.5% PEG lipid and 32.5% structural lipid. In some embodiments, the ionizable cationic lipid may be any cationic lipid described herein such as, but not limited to, DLin-KC2-DMA, DLin-MC3-DMA and L319.

In some embodiments, the lipid nanoparticle formulations described herein may comprise a ionizable cationic lipid, a non-cationic lipid, a PEG lipid and a structural lipid. As a non-limiting example, the lipid nanoparticle comprise 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprise 55% of the cationic lipid L319, 10% of the non-cationic lipid DSPC, 2.5% of the PEG lipid PEG-DMG and 32.5% of the structural lipid cholesterol.

Relative amounts of the active ingredient, the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the RNA vaccine composition may comprise the polynucleotide described herein, formulated in a lipid nanoparticle comprising MC3, Cholesterol, DSPC and PEG2000-DMG, the buffer trisodium citrate, sucrose and water for injection. As a non-limiting example, the composition comprises: 2.0 mg/mL of drug substance (e.g., polynucleotides encoding H10N8 influenza virus), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

### Liposomes, Lipoplexes, and Lipid Nanoparticles

The RNA vaccines of the invention can be formulated using one or more liposomes, lipoplexes, or lipid nanoparticles. In some embodiments, the RNA vaccine comprises one or more RNA polynucleotides comprising one or more open reading frames encoding one or more of HCMV antigenic polypeptides gB, gH, gL, UL128, UL130, UL131, and pp65. In some embodiments, all of the RNA polynucleotide components of the vaccine are formulated in the same liposome, lipoplex or lipid nanoparticle. In other embodiments, one or more of the RNA polynucleotide components of the vaccine are formulated in different liposomes, lipoplexes or lipid nanoparticles. In other embodiments, each of RNA polynucleotide components of the vaccine is formulated in a different liposome, lipoplex or lipid nanoparticle. In some embodiments, an RNA vaccine comprises RNA polynucleotides encoding gB, gH, gL, UL128, UL130 and UL131. The RNA polynucleotides encoding gB, gH, gL, UL128, UL130 and UL131 can be formulated in one or more liposomes, lipoplexes, or lipid nanoparticles. In certain embodiments, RNA polynucleotides encoding gB, gH, gL, UL128, UL130 and UL131 are all included in the same liposome, lipoplexe, or lipid nanoparticle. In some embodiments, an RNA vaccine further comprises an RNA polynucleotide encoding pp65. In some embodiments, the pp65 polypeptide contains a deletion of amino acids 435-438. The RNA polynucleotide encoding pp65 can be formulated with the other RNA components of the vaccine or separately from the other RNA components of the vaccine. In some embodiments, an RNA polynucleotide encoding pp65 is formulated in a separate vaccine. In some embodiments, RNA polynucleotides encoding gB, gH, gL, UL128, UL130 and UL131 are all included in the same liposome, lipoplexe, or lipid nanoparticle, while an RNA polynucleotide encoding pp65 is formulated in a separate liposome, lipoplexe, or lipid nanoparticle. When RNA polynucleotides are formulated in separate liposomes, lipoplexes, or lipid nanoparticles, they can be administered together or separately. In some embodiments, a liposome, lipoplexe, or lipid nanoparticle comprising an RNA polynucleotide encoding pp65 is administered before a liposome, lipoplexe, or lipid nanoparticle comprising RNA polynucleotides encoding gB, gH, gL, UL128, UL130, and UL131. In some embodiments, a liposome, lipoplexe, or lipid nanoparticle comprising an RNA polynucleotide encoding pp65 is administered before a liposome, lipoplexe, or lipid nanoparticle comprising RNA polynucleotides encoding gB, gH, gL, UL128, UL130, UL131, and pp65.

In some embodiments, pharmaceutical compositions of RNA vaccines include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, PA).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In a preferred embodiment, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In some embodiments, pharmaceutical compositions may include liposomes which may be formed to deliver polynucleotides which may encode at least one immunogen (antigen) or any other polypeptide of interest. The RNA vaccine may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In another embodiment, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are herein incorporated by reference in its entirety.

In another embodiment, the polynucleotide which may encode an immunogen (antigen) may be formulated in a cationic oil-in-water emulsion where the emulsion particle comprises an oil core and a cationic lipid which can interact with the polynucleotide anchoring the molecule to the emulsion particle (see International Pub. No. WO2012006380; herein incorporated by reference in its entirety).

In some embodiments, the RNA vaccines may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety.

In another embodiment, the lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; the contents of each of which is herein incorporated by reference in their entirety). In another embodiment, the polynucleotides encoding an immunogen may be formulated in a lipid vesicle which may have crosslinks between functionalized lipid bilayers (see U.S. Pub. No. 20120177724, the contents of which is herein incorporated by reference in its entirety).

In some embodiments, the polylnucleotides may be formulated in a lipsome as described in International Patent Publication No. WO2013086526, the contents of which is herein incorporated by reference in its entirety. The RNA vaccines may be encapsulated in a liposome using reverse pH gradients and/or optimized internal buffer compositions as described in International Patent Publication No. WO2013086526, the contents of which is herein incorporated by reference in its entirety.

In some embodiments, the RNA vaccine pharmaceutical compositions may be formulated in liposomes such as, but not limited to, DiLa2 liposomes (Marina Biotech. Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the cationic lipid may be a low molecular weight cationic lipid such as those described in US Patent Application No. 20130090372, the contents of which are herein incorporated by reference in its entirety.

In some embodiments, the RNA vaccines may be formulated in a lipid vesicle which may have crosslinks between functionalized lipid bilayers.

In some embodiments, the RNA vaccines may be formulated in a liposome comprising a cationic lipid. The liposome may have a molar ratio of nitrogen atoms in the cationic lipid to the phophates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

In some embodiments, the RNA vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In another embodiment, the RNA vaccines may be formulated in a lipid-polycation complex which may further include a non-cationic lipid such as, but not limited to, cholesterol or dioleoyl phosphatidylethanolamine (DOPE).

In some embodiments, the RNA vaccines may be formulated in an aminoalcohol lipidoid. Aminoalcohol lipidoids which may be used in the present invention may be prepared by the methods described in U.S. Pat. No. 8,450,298, herein incorporated by reference in its entirety.

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about about 5:1 to about 20:1, from about 10:1 to about 25:1, from about 15:1 to about 30:1 and/or at least 30:1.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(o-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the RNA vaccines may be formulated in a lipid nanoparticle such as those described in International Publication No. WO2012170930, the contents of which is herein incorporated by reference in its entirety.

In some embodiments, the RNA vaccine formulation comprising the polynucleotide is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the formulation includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in its entirety)

In some embodiments, the formulations of the inventions include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, the formulations of the inventions include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, the formulations of the inventions include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, the formulations of the inventions include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In some embodiments, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In some embodiments, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In some embodiments, the formulations of the inventions include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In some embodiments, the formulations of the inventions include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In some embodiments, the formulations of the inventions include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in its entirety), about 7.5% of the neutral lipid, about 31.5% of the sterol, and about 3.5% of the PEG or PEG-modified lipid on a molar basis.

In preferred embodiments, lipid nanoparticle formulation consists essentially of a lipid mixture in molar ratios of about 20-70% ionizable cationic lipid: 5-45% neutral lipid: 20-55% cholesterol: 0.5-15% PEG-modified lipid; more preferably in a molar ratio of about 20-60% ionizable cationic lipid: 5-25% neutral lipid: 25-55% cholesterol: 0.5-15% PEG-modified lipid.

In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Cho1/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Exemplary lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, the lipid nanoparticle formulations described herein may comprise a ionizable cationic lipid, a PEG lipid and a structural lipid and optionally comprise a non-cationic lipid. As a non-limiting example, the lipid nanoparticle may comprise about 40-60% of ionizable cationic lipid, about 5-15% of a non-cationic lipid, about 1-2% of a PEG lipid and about 30-50% of a structural lipid. As another non-limiting example, the lipid nanoparticle may comprise about 50% ionizable cationic lipid, about 10% non-cationic lipid, about 1.5% PEG lipid and about 38.5% structural lipid. As yet another non-limiting example, the lipid nanoparticle may comprise about 55% ionizable cationic lipid, about 10% non-cationic lipid, about 2.5% PEG lipid and about 32.5% structural lipid. In some embodiments, the ionizable cationic lipid may be any cationic lipid described herein such as, but not limited to. DLin-KC2-DMA, DLin-MC3-DMA and L319.

In some embodiments, the lipid nanoparticle formulations described herein may be 4 component lipid nanoparticles. The lipid nanoparticle may comprise a ionizable cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In some embodiments, the cationic lipid may be any cationic lipid described herein such as, but not limited to. DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

In some embodiments, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836; the contents of each of which are herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365. WO2012044638 and WO2013116126 or US Patent Publication No. US20130178541 and US20130225836; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-V1 of US Patent Publication No. US20100036115, formula I of US Patent Publication No US20130123338; each of which is herein incorporated by reference in their entirety. As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-1 6, 19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethylricosa-14,17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconma-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine. (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyleptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimetbylhentriacont-22-en-10-amine, (16Z)-N,N-dimethylpenmacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimetbylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In some embodiments, the lipid may be a cleavable lipid such as those described in International Publication No. WO2012170889, herein incorporated by reference in its entirety.

In another embodiment, the lipid may be a cationic lipid such as, but not limited to, Formula (I) of U.S. Patent Application No. US20130064894, the contents of which are herein incorporated by reference in its entirety.

In some embodiments, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354; the contents of each of which are herein incorporated by reference in their entirety.

In another embodiment, the cationic lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In some embodiments, the LNP formulations of the RNA vaccines may contain PEG-c-DOMG at 3% lipid molar ratio. In another embodiment, the LNP formulations RRNA vaccines may contain PEG-c-DOMG at 1.5% lipid molar ratio.

In some embodiments, the pharmaceutical compositions of the RNA vaccines may include at least one of the PEGylated lipids described in International Publication No. WO2012099755, the contents of which is herein incorporated by reference in its entirety.

In some embodiments, the LNP formulation may contain PEG-DMG 2000 (1,2-dimyristoyl-sn-glycero-3-phophoethanolamine-N-[methoxy(polyethylene glycol)-2000). In some embodiments, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety).

In some embodiments, the LNP formulation may be formulated by the methods described in International Publication Nos. WO2011127255 or WO2008103276, the contents of each of which is herein incorporated by reference in their entirety. As a non-limiting example, the RNA vaccines described herein may be encapsulated in LNP formulations as described in WO2011127255 and/or WO2008103276; each of which is herein incorporated by reference in their entirety.

In some embodiments, the RNA vaccines described herein may be formulated in a nanoparticle to be delivered by a parenteral route as described in U.S. Pub. No. US20120207845; the contents of which are herein incorporated by reference in its entirety.

In some embodiments, the RNA vaccines may be formulated in a lipid nanoparticle made by the methods described in US Patent Publication No US20130156845 or International Publication No WO2013093648 or WO2012024526, each of which is herein incorporated by reference in its entirety.

The lipid nanoparticles described herein may be made in a sterile environment by the system and/or methods described in US Patent Publication No. US20130164400, herein incorporated by reference in its entirety.

In some embodiments, the LNP formulation may be formulated in a nanoparticle such as a nucleic acid-lipid particle described in U.S. Pat. No. 8,492,359, the contents of which are herein incorporated by reference in its entirety. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle may be the polynucleotides described herein and/or are known in the art.

In some embodiments, the LNP formulation may be formulated by the methods described in International Publication Nos. WO2011127255 or WO2008103276, the contents of each of which are herein incorporated by reference in their entirety. As a non-limiting example, modified RNA described herein may be encapsulated in LNP formulations as described in WO2011127255 and/or WO2008103276; the contents of each of which are herein incorporated by reference in their entirety.

In some embodiments. LNP formulations described herein may comprise a polycationic composition. As a non-limiting example, the polycationic composition may be selected from formula 1-60 of US Patent Publication No. US20050222064; the content of which is herein incorporated by reference in its entirety. In another embodiment, the LNP formulations comprising a polycationic composition may be used for the delivery of the modified RNA described herein in vivo and/or in vitro.

In some embodiments, the LNP formulations described herein may additionally comprise a permeability enhancer molecule. Non-limiting permeability enhancer molecules are described in US Patent Publication No. US20050222064; the content of which is herein incorporated by reference in its entirety.

In some embodiments, the RNA vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the RNA vaccines may be formulated in a lyophilized gel-phase liposomal composition as described in US Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Application No. WO2013033438 or US Patent Publication No. US20130196948, the contents of each of which are herein incorporated by reference in its entirety. As a non-limiting example, the phosphate conjugates may include a compound of any one of the formulas described in International Application No. WO2013033438, herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In one aspect, polymer conjugates with the polynucleotides of the present invention may be made using the methods and/or segmented polymeric reagents described in U.S. Patent Application No. 20130072709, herein incorporated by reference in its entirety. In another aspect, the polymer conjugate may have pendant side groups comprising ring moieties such as, but not limited to, the polymer conjugates described in US Patent Publication No. US20130196948, the contents of which is herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In one aspect, the conjugate may be a “self” peptide designed from the human membrane protein CD47 (e.g., the “self” particles described by Rodriguez et al (Science 2013 339, 971-975), herein incorporated by reference in its entirety). As shown by Rodriguez et al. the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, the RNA vaccines of the present invention are formulated in nanoparticles which comprise a conjugate to enhance the delivery of the nanoparticles of the present invention in a subject. The conjugate may be the CD47 membrane or the conjugate may be derived from the CD47 membrane protein, such as the “self” peptide described previously. In another aspect the nanoparticle may comprise PEG and a conjugate of CD47 or a derivative thereof. In yet another aspect, the nanoparticle may comprise both the “self” peptide described above and the membrane protein CD47.

In another aspect, a “self” peptide and/or CD47 protein may be conjugated to a virus-like particle or pseudovirion, as described herein for delivery of the RNA vaccines of the present invention.

In another embodiment, RNA vaccine pharmaceutical compositions comprising the polynucleotides of the present invention and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443, the contents of which are herein incorporated by reference in its entirety.

The nanoparticle formulations may be a carbohydrate nanoparticle comprising a carbohydrate carrier and a RNA vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in its entirety).

Nanoparticle formulations of the present invention may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA vaccines within the central nervous system. As a non-limiting example nanoparticles comprising a hydrophilic coating and methods of making such nanoparticles are described in US Patent Publication No. US20130183244, the contents of which are herein incorporated by reference in its entirety.

In some embodiments, the lipid nanoparticles of the present invention may be hydrophilic polymer particles. Non-limiting examples of hydrophilic polymer particles and methods of making hydrophilic polymer particles are described in US Patent Publication No. US20130210991, the contents of which are herein incorporated by reference in its entirety.

In another embodiment, the lipid nanoparticles of the present invention may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the internal ester linkage may be located on either side of the saturated carbon.

In some embodiments, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limted to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosla tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in its entirety. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in its entirety). A non-limiting scalable method to produce nanoparticles which can penetrate human mucus is described by Xu et al. (See e.g., J Control Release 2013, 170(2):279-86; the contents of which are herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., US Publication 20100215580 and US Publication 20080166414 and US20130164343; the contents of each of which is herein incorporated by reference in their entirety).

In some embodiments, the mucus penetrating lipid nanoparticles may comprise at least one polynucleotide described herein. The polynucleotide may be encapsulated in the lipid nanoparticle and/or disposed on the surface of the paricle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In another embodiment, the mucus penetrating lipid nanoparticles may be a hypotonic formulation comprising a mucosal penetration enhancing coating. The formulation may be hypotonice for the epithelium to which it is being delivered. Non-limiting examples of hypotonic formulations may be found in International Patent Publication No. WO2013110028, the contents of which are herein incorporated by reference in its entirety.

In some embodiments, in order to enhance the delivery through the mucosal barrier the RNA vaccine formulation may comprise or be a hypotonic solution. Hypotonic solutions were found to increase the rate at which mucoinert particles such as, but not limited to, mucus-penetrating particles, were able to reach the vaginal epithelial surface (See e.g., Ensign et al. Biomaterials 2013 34(28):6922-9; the contents of which is herein incorporated by reference in its entirety).

In some embodiments, the RNA vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344: Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673: Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Cuff Drug Discov Technol. 2011 8:197-206; Musacchio and Torchilin, Front Biosci. 2011 16:1388-1412; Yu et al., Mol Membr Biol. 2010 27:286-298: Patil et al., Crit Rev Ther Drug Carrier Syst. 2008 25:1-61; Benoit et al., Biomacromolecules. 2011 12:2708-2714: Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68: Peer et al., Proc Natl Acad Sci U S A. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630: Peer and Lieberman, Gene Ther. 2011 18:1127-1133; all of which are incorporated herein by reference in its entirety).

In some embodiments, the RNA vaccine is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; the contents of which are herein incorporated by reference in its entirety). As a non-limiting example, the SLN may be the SLN described in International Patent Publication No. WO2013105101, the contents of which are herein incorporated by reference in its entirety. As another non-limiting example, the SLN may be made by the methods or processes described in International Patent Publication No. WO2013105101, the contents of which are herein incorporated by reference in its entirety.

Liposomes, lipoplexes, or lipid nanoparticles may be used to improve the efficacy of polynucleotides directed protein production as these formulations may be able to increase cell transfection by the RNA vaccine; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the polynucleotide.

In some embodiments, the RNA vaccines of the present invention can be formulated for controlled release and/or targeted delivery. As used herein, “controlled release” refers to a pharmaceutical composition or compound release profile that conforms to a particular pattern of release to effect a therapeutic outcome. In some embodiments, the RRNA vaccines may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

In another embodiment, the RNA vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL). HYLENEX® (Halozyme Therapeutics, San Diego CA), surgical sealants such as fibrinogen polymers (Ethicon Inc. Cornelia, GA), TISSELL® (Baxter International, Inc Deerfield, IL), PEG-based sealants, and COSEAL® (Baxter International, Inc Deerfield, IL).

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the the RNA vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the RNA vaccine controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In some embodiments, the RNA vaccine controlled release and/or targeted delivery formulation comprising at least one polynucleotide may comprise at least one PEG and/or PEG related polymer derivatives as described in U.S. Pat. No. 8,404,222, herein incorporated by reference in its entirety.

In another embodiment, the RNA vaccine controlled release delivery formulation comprising at least one polynucleotide may be the controlled release polymer system described in US20130130348, herein incorporated by reference in its entirety.

In some embodiments, the the RNA vaccines of the present invention may be encapsulated in a therapeutic nanoparticle, referred to herein as “therapeutic nanoparticle RRNA vaccines.” Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in US Pub No. US20120140790, the contents of which is herein incorporated by reference in its entirety.

In some embodiments, the therapeutic nanoparticle RNA vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the the polynucleotides of the present invention (see International Pub No. 2010075072 and US Pub No. US20100216804, US20110217377 and US20120201859, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety).

In some embodiments, the therapeutic nanoparticle RNA vaccines may be formulated to be target specific. As a non-limiting example, the thereapeutic nanoparticles may include a corticosteroid (see International Pub. No. WO2011084518; herein incorporated by reference in its entirety). As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In some embodiments, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the therapeutic nanoparticle comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In another embodiment, the diblock copolymer may comprise the diblock copolymers described in European Patent Publication No. the contents of which are herein incorporated by reference in its entirety. In yet another embodiment, the diblock copolymer may be a high-X diblock copolymer such as those described in International Patent Publication No. WO2013120052, the contents of which are herein incorporated by reference in its entirety.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in its entirety). In yet another non-limiting example, the therapeutic nanoparticle is a stealth nanoparticle or a target-specific stealth nanoparticle as described in US Patent Publication No. US20130172406, the contents of which are herein incorporated by reference in its entirety.

In some embodiments, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In yet another non-limiting example, the lipid nanoparticle comprises the block copolymer PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety). The RNA vaccines of the present invention may be formulated in lipid nanoparticles comprising the PEG-PLGA-PEG block copolymer.

In some embodiments, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In some embodiments, the block copolymers described herein may be included in a polyion complex comprising a non-polymeric micelle and the block copolymer. (See e.g., U.S. Pub. No. 20120076836; herein incorporated by reference in its entirety).

In some embodiments, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In some embodiments, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or US Patent Publication No US20130121954, the contents of which are herein incorporated by reference in its entirety. In one aspect, the poly(vinyl ester) polymers may be conjugated to the polynucleotides described herein. In another aspect, the poly(vinyl ester) polymer which may be used in the present invention may be those described in, herein incorporated by reference in its entirety.

In some embodiments, the therapeutic nanoparticle may comprise at least one diblock copolymer. The diblock copolymer may be, but it not limited to, a poly(lactic) acid-poly(ethylene)glycol copolymer (see e.g., International Patent Publication No. WO2013044219; herein incorporated by reference in its entirety). As a non-limiting example, the therapeutic nanoparticle may be used to treat cancer (see International publication No. WO2013044219; herein incorporated by reference in its entirety).

In some embodiments, the therapeutic nanoparticles may comprise at least one cationic polymer described herein and/or known in the art.

In some embodiments, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof.

In another embodiment, the nanoparticles described herein may comprise an amine cationic lipid such as those described in International Patent Application No. WO2013059496, the contents of which are herein incorporated by reference in its entirety. In one aspect the cationic lipids may have an amino-amine or an amino-amide moiety.

In some embodiments, the therapeutic nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In some embodiments, the therapeutic nanoparticle may be formulated in an aqueous solution which may be used to target cancer (see International Pub No. WO2011084513 and US Pub No. US20110294717, each of which is herein incorporated by reference in their entirety).

In some embodiments, the therapeutic nanoparticle RNA vaccines, e.g., therapeutic nanoparticles comprising at least one RNA vaccine may be formulated using the methods described by Podobinski et al in U.S. Pat. No. 8,404,799, the contents of which are herein incorporated by reference in its entirety.

In some embodiments, the RNA vaccines may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, each of which is herein incorporated by reference in their entirety. In another embodiment, the synthetic nanocarrier formulations may be lyophilized by methods described in International Pub. No. WO2011072218 and U.S. Pat. No. 8,211,473; the content of each of which is herein incorporated by reference in their entirety. In yet another embodiment, formulations of the present invention, including, but not limited to, synthetic nanocarriers, may be lyophilized or reconstituted by the methods described in US Patent Publication No. US20130230568, the contents of which are herein incorporated by reference in its entirety.

In some embodiments, the synthetic nanocarriers may contain reactive groups to release the polynucleotides described herein (see International Pub. No. WO20120952552 and US Pub No. US20120171229, each of which is herein incorporated by reference in their entirety).

In some embodiments, the synthetic nanocarriers may contain an immunostimulatory agent to enhance the immune response from delivery of the synthetic nanocarrier. As a non-limiting example, the synthetic nanocarrier may comprise a Th1 immunostimulatory agent which may enhance a Th1-based response of the immune system (see International Pub No. WO2010123569 and US Pub. No. US20110223201, each of which is herein incorporated by reference in its entirety).

In some embodiments, the synthetic nanocarriers may be formulated for targeted release. In some embodiments, the synthetic nanocarrier is formulated to release the polynucleotides at a specified pH and/or after a desired time interval. As a non-limiting example, the synthetic nanoparticle may be formulated to release the RNA vaccines after 24 hours and/or at a pH of 4.5 (see International Pub. Nos. WO2010138193 and WO2010138194 and US Pub Nos. US20110020388 and US20110027217, each of which is herein incorporated by reference in their entireties).

In some embodiments, the synthetic nanocarriers may be formulated for controlled and/or sustained release of the polynucleotides described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the RNA vaccine may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In some embodiments, the synthetic nanocarrier may be formulated for use as a vaccine. In some embodiments, the synthetic nanocarrier may encapsulate at least one polynucleotide which encode at least one antigen. As a non-limiting example, the synthetic nanocarrier may include at least one antigen and an excipient for a vaccine dosage form (see International Pub No. WO2011150264 and US Pub No. US20110293723, each of which is herein incorporated by reference in their entirety). As another non-limiting example, a vaccine dosage form may include at least two synthetic nanocarriers with the same or different antigens and an excipient (see International Pub No. WO2011150249 and US Pub No. US20110293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

In some embodiments, the synthetic nanocarrier may comprise at least one polynucleotide which encodes at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (See e.g, U.S. Pat. No. 8,241,610; herein incorporated by reference in its entirety). In another embodiment, the synthetic nanocarrier may comprise at least one polynucleotide and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety.

In some embodiments, the synthetic nanocarrier may encapsulate at least one polynucleotide which encodes a peptide, fragment or region from a virus. As a non-limiting example, the synthetic nanocarrier may include, but is not limited to, the nanocarriers described in International Pub No. WO2012024621, WO201202629, WO2012024632 and US Pub No. US20120064110, US20120058153 and US20120058154, each of which is herein incorporated by reference in their entirety.

In some embodiments, the synthetic nanocarrier may be coupled to a polynucleotide which may be able to trigger a humoral and/or cytotoxic T lymphocyte (CTL) response (See e.g., International Publication No. WO2013019669, herein incorporated by reference in its entirety).

In some embodiments, the RNA vaccine may be encapsulated in, linked to and/or associated with zwitterionic lipids. Non-limiting examples of zwitterionic lipids and methods of using zwitterionic lipids are described in US Patent Publication No. US20130216607, the contents of which are herein incorporated by reference in its entirety. In one aspect, the zwitterionic lipids may be used in the liposomes and lipid nanoparticles described herein.

In some embodiments, the RNA vaccine may be formulated in colloid nanocarriers as described in US Patent Publication No. US20130197100, the contents of which are herein incorporated by reference in its entirety.

In some embodiments, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343: herein incorporated by reference in its entirety.

In some embodiments, LNPs comprise the lipid KL52 (an amino-lipid disclosed in U.S. Application Publication No. 2012/0295832 expressly incorporated herein by reference in its entirety). Activity and/or safety (as measured by examining one or more of ALT/AST, white blood cell count and cytokine induction) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments. RNA vaccine may be delivered using smaller LNPs. Such particles may comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, and less than 975 um.

In another embodiment, RNA vaccines may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1: e37; Chen, D. et al., Rapid discovery of potent siRNA-containing lipid nanoparticles enabled by controlled microfluidic formulation. J Am Chem Soc. 2012. 134(16):6948-51; each of which is herein incorporated by reference in its entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety.

In some embodiments, the RNA vaccine of the present invention may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (UMM) from the Institut für Mikrotechnik Mainz GmbH, Mainz Germany).

In some embodiments, the RNA vaccines of the present invention may be formulated in lipid nanoparticles created using microfluidic technology (see Whitesides, George M. The Origins and the Future of Microfluidics. Nature, 2006 442: 368-373; and Abraham et al. Chaotic Mixer for Microchannels. Science, 2002 295: 647-651; each of which is herein incorporated by reference in its entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651; which is herein incorporated by reference in its entirety).

In some embodiments, the RNA vaccines of the present invention may be formulated in lipid nanoparticles created using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In some embodiments, the RNA vaccines of the invention may be formulated for delivery using the drug encapsulating microspheres described in International Patent Publication No. WO2013063468 or U.S. Pat. No. 8,440,614, each of which is herein incorporated by reference in its entirety. The microspheres may comprise a compound of the formula (I), (II), (III), (IV), (V) or (V1) as described in International Patent Publication No. WO2013063468, the contents of which are herein incorporated by reference in its entirety. In another aspect, the amino acid, peptide, polypeptide, lipids (APPL) are useful in delivering the RNA vaccines of the invention to cells (see International Patent Publication No. WO2013063468, the contents of which is herein incorporated by reference in its entirety).

In some embodiments, the RNA vaccines of the invention may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm.

In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In one aspect, the lipid nanoparticle may be a limit size lipid nanoparticle described in International Patent Publication No. WO2013059922, the contents of which are herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may comprise a polyethylene glycol-lipid such as, but not limited to, DLPE-PEG, DMPE-PEG, DPPC-PEG and DSPE-PEG.

In some embodiments, the RNA vaccines may be delivered, localized and/or concentrated in a specific location using the delivery methods described in International Patent Publication No. WO2013063530, the contents of which are herein incorporated by reference in its entirety. As a non-limiting example, a subject may be administered an empty polymeric particle prior to, simultaneously with or after delivering the RNA vaccines to the subject. The empty polymeric particle undergoes a change in volume once in contact with the subject and becomes lodged, embedded, immobilized or entrapped at a specific location in the subject.

In some embodiments, the RNA vaccines may be formulated in an active substance release system (See e.g., US Patent Publication No. US20130102545, the contents of which is herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In some embodiments, the RNA vaccines may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, herein incorporated by reference in its entirety. As another non-limiting example, the nanoparticle described in International Patent Publication No. WO2013052167, herein incorporated by reference in its entirety, may be used to deliver the RNA vaccines described herein.

In some embodiments, the RNA vaccines may be formulated in porous nanoparticle-supported lipid bilayers (protocells). Protocells are described in International Patent Publication No. WO2013056132, the contents of which are herein incorporated by reference in its entirety.

In some embodiments, the RNA vaccines described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in its entirety. As another non-limiting example, the polymer nanoparticle for oral and parenteral formulations may be made by the methods described in European Patent No. EP2073848B1, the contents of which are herein incorporated by reference in its entirety.

In another embodiment, the RNA vaccines described herein may be formulated in nanoparticles used in imaging. The nanoparticles may be liposome nanoparticles such as those described in US Patent Publication No US20130129636, herein incorporated by reference in its entirety. As a non-limiting example, the liposome may comprise gadolinium(III)2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component (see e.g., US Patent Publication No US20130129636, the contents of which is herein incorporated by reference in its entirety).

In some embodiments, the nanoparticles which may be used in the present invention are formed by the methods described in U.S. Patent Application No. US20130130348, the contents of which is herein incorporated by reference in its entirety.

The nanoparticles of the present invention may further include nutrients such as, but not limited to, those which deficiencies can lead to health hazards from anemia to neural tube defects (see e.g. the nanoparticles described in International Patent Publication No WO2013072929, the contents of which is herein incorporated by reference in its entirety). As a non-limiting example, the nutrient may be iron in the form of ferrous, ferric salts or elemental iron, iodine, folic acid, vitamins or micronutrients.

In some embodiments, the RNA vaccines of the present invention may be formulated in a swellable nanoparticle. The swellable nanoparticle may be, but is not limited to, those described in U.S. Pat. No. 8,440,231, the contents of which is herein incorporated by reference in its entirety. As a non-limiting embodiment, the swellable nanoparticle may be used for delivery of the RNA vaccines of the present invention to the pulmonary system (see e.g., U.S. Pat. No. 8,440,231, the contents of which is herein incorporated by reference in its entirety).

The RNA vaccines of the present invention may be formulated in polyanhydride nanoparticles such as, but not limited to, those described in U.S. Pat. No. 8,449,916, the contents of which is herein incorporated by reference in its entirety.

The nanoparticles and microparticles of the present invention may be geometrically engineered to modulate macrophage and/or the immune response. In one aspect, the geometrically engineered particles may have varied shapes, sizes and/or surface charges in order to incorporated the polynucleotides of the present invention for targeted delivery such as, but not limited to, pulmonary delivery (see e.g., International Publication No WO2013082111, the contents of which is herein incorporated by reference in its entirety). Other physical features the geometrically engineering particles may have include, but are not limited to, fenestrations, angled arms, asymmetry and surface roughness, charge which can alter the interactions with cells and tissues. As a non-limiting example, nanoparticles of the present invention may be made by the methods described in International Publication No WO2013082111, the contents of which is herein incorporated by reference in its entirety.

In some embodiments, the nanoparticles of the present invention may be water soluble nanoparticles such as, but not limited to, those described in International Publication No. WO2013090601, the contents of which is herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments the nanoparticles of the present invention may be developed by the methods described in US Patent Publication No. US20130172406, the contents of which are herein incorporated by reference in its entirety.

In some embodiments, the nanoparticles of the present invention are stealth nanoparticles or target-specific stealth nanoparticles such as, but not limited to, those described in US Patent Publication No. US20130172406; the contents of which is herein incorporated by reference in its entirety. The nanoparticles of the present invention may be made by the methods described in US Patent Publication No. US20130172406, the contents of which are herein incorporated by reference in its entirety.

In another embodiment, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In some embodiments, the nanoparticle may be a nanoparticle-nucleic acid hybrid structure having a high density nucleic acid layer. As a non-limiting example, the nanoparticle-nucleic acid hybrid structure may made by the methods described in US Patent Publication No. US20130171646, the contents of which are herein incorporated by reference in its entirety. The nanoparticle may comprise a nucleic acid such as, but not limited to, polynucleotides described herein and/or known in the art.

At least one of the nanoparticles of the present invention may be embedded in in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in its entirety.

In some embodiments the RNA (e.g., mRNA) vaccine may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP²² derived or analog peptides, Pestivirus Ems, HSV, VP²² (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s). Antennapedia-derived peptides (particularly from Drosophila antennapedia), pAntp, pIs1, FGF, Lactoferrin, Transportan, Buforin-2, Bac715-24, SynB, SynB(1), pVEC, hCT-derived peptides, SAP, histones, cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS:

Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammo-nium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole), etc.

In other embodiments the RNA (e.g., mRNA) vaccine is not associated with a cationic or polycationic compounds.

In some embodiments, a nanoparticle comprises compounds of Formula (I):

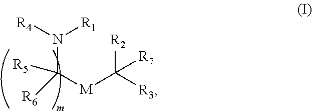

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)O R, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—,
—N(R′)C(O)—, —C(O)—, —C(S)—, —C(S)S—, —SC(S)—, —CH(OH)—, —P(O)(OR′)O—, —S(O)₂—, —S —S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
—CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR,
—O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)O R, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆, alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
—CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR,
—O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR,
and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
—CHQR, —CQ(R)₂, and unsubstituted C₁₋₆ alkyl, where Q is selected from a C₃ carbocycle, a 5- to 14-membered heteroaryl having one or more heteroatoms selected from N, O, and S, —OR,
—O(CH₂)ₙN(R)₂. —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)˜OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R, is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

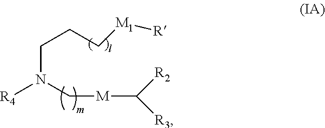

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

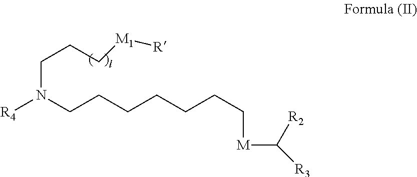

or a salt or isomer thereof, wherein 1 is selected from 1, 2, 3, 4, and 5: M₁ is a bond or M′; R₄ is unsubstituted C₁₋₃ alkyl,
or —(CH₂)˜Q, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

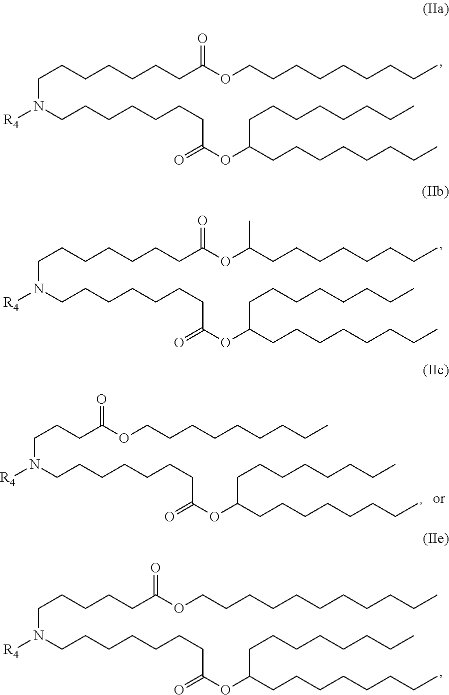

or a salt or isomer thereof, wherein R₄ is as described herein.

In some embodiments, a subset of compounds of Formula (I) includes those of Formula (IId):

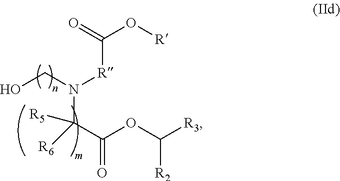

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, a subset of compounds of Formula (I) includes those of Formula (IIa), (IIb), (IIc), or (IIe):

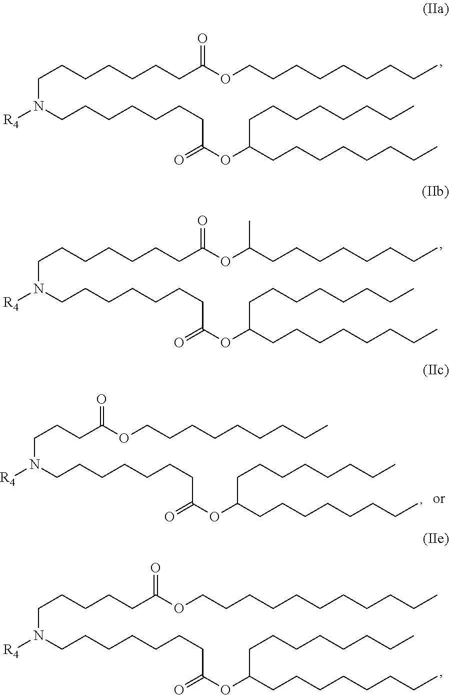

or a salt or isomer thereof, wherein R₄ is as described herein.

In some embodiments, a subset of compounds of Formula (I) includes those of Formula (IId):

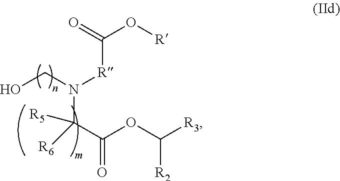

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, the compound of Formula (I) is selected from the group consisting of:

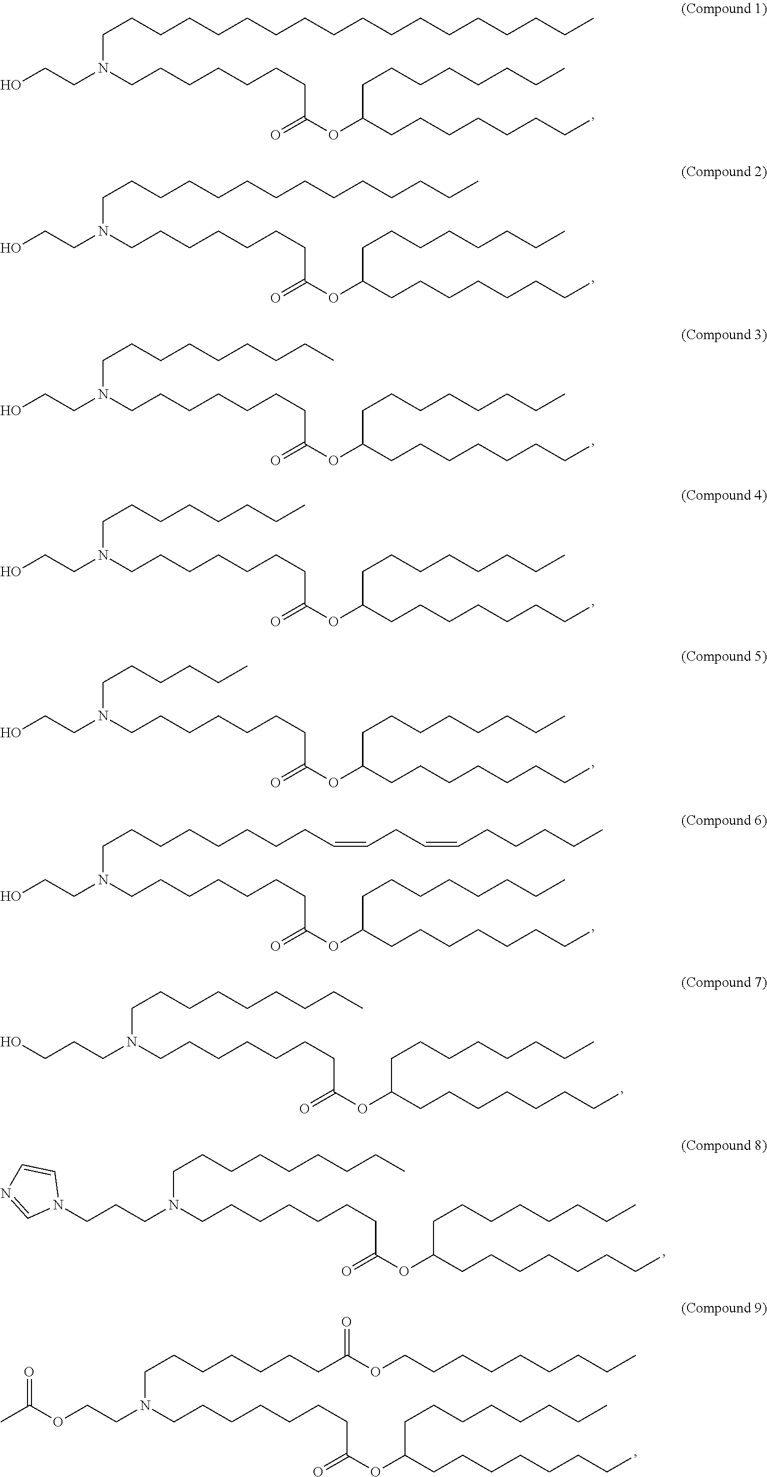

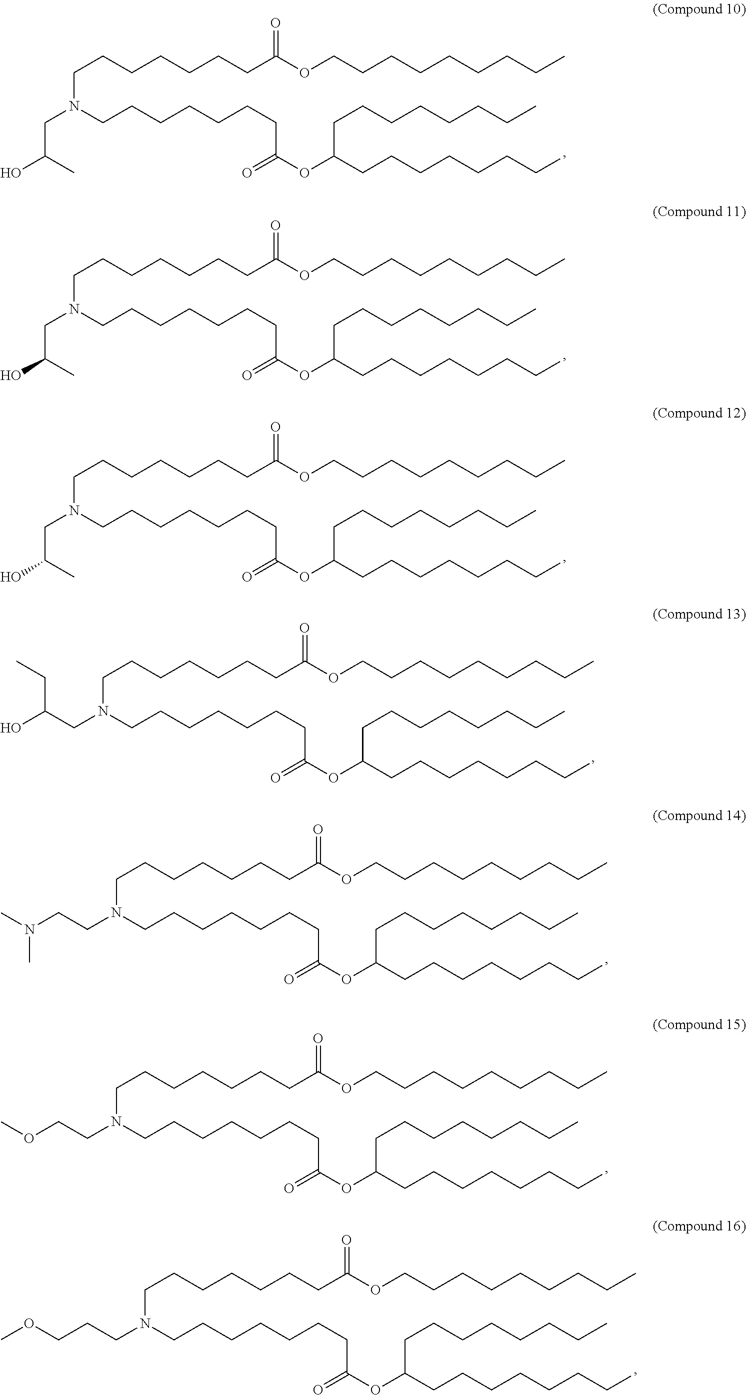

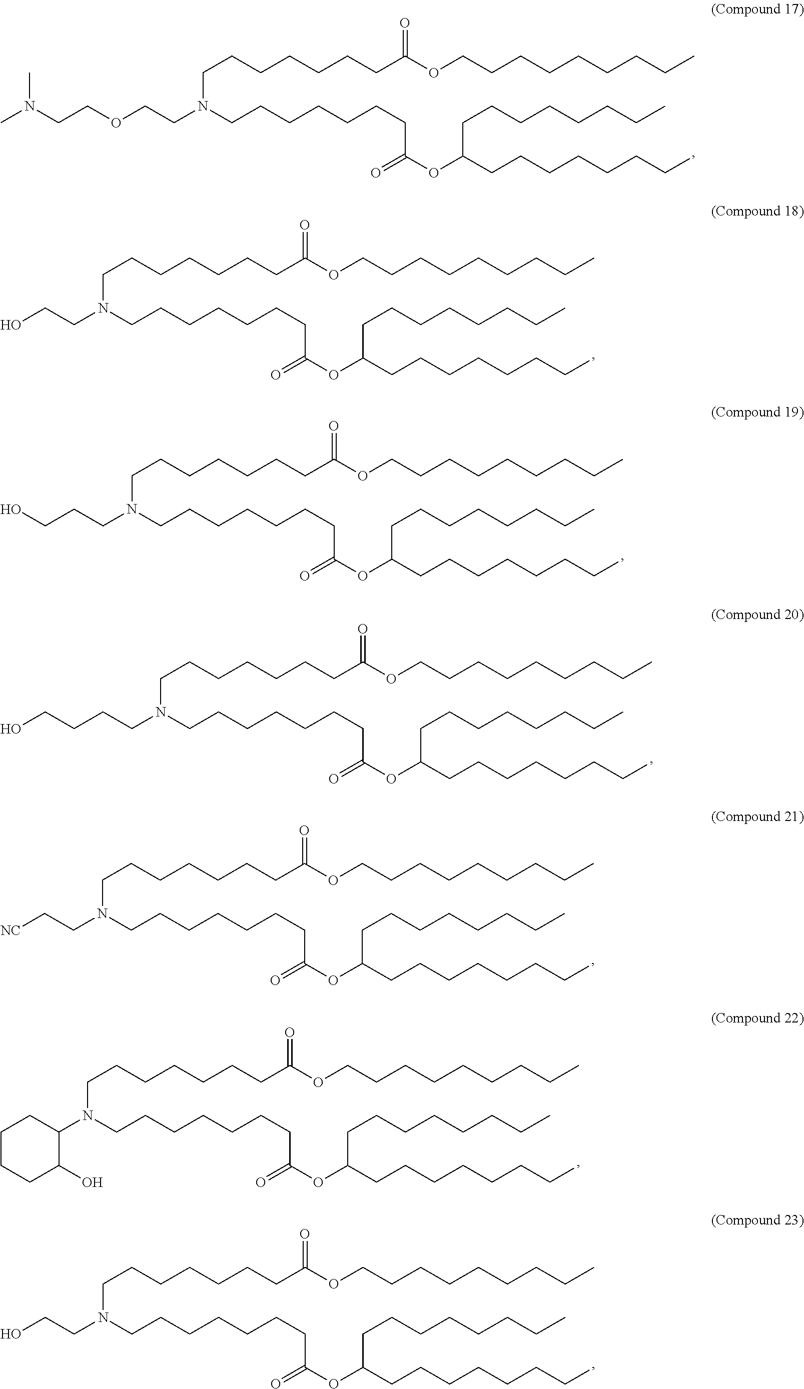

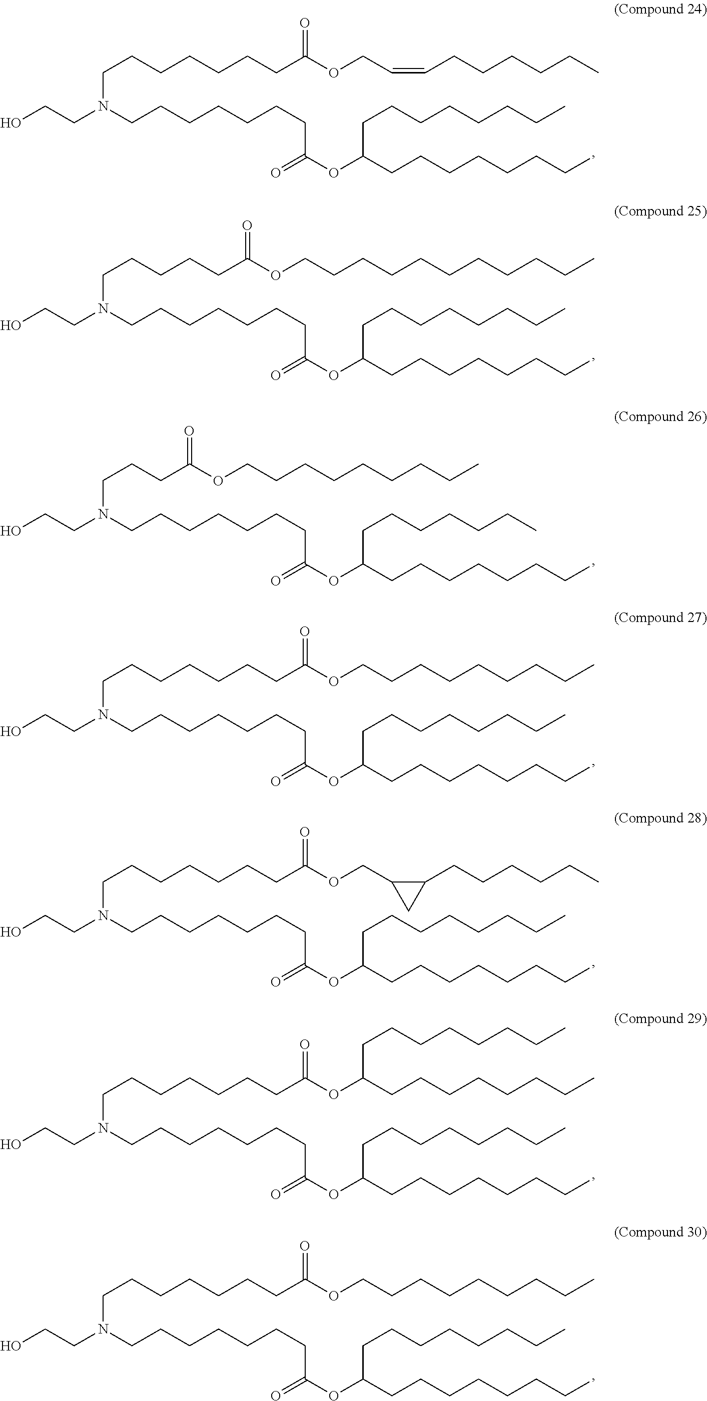

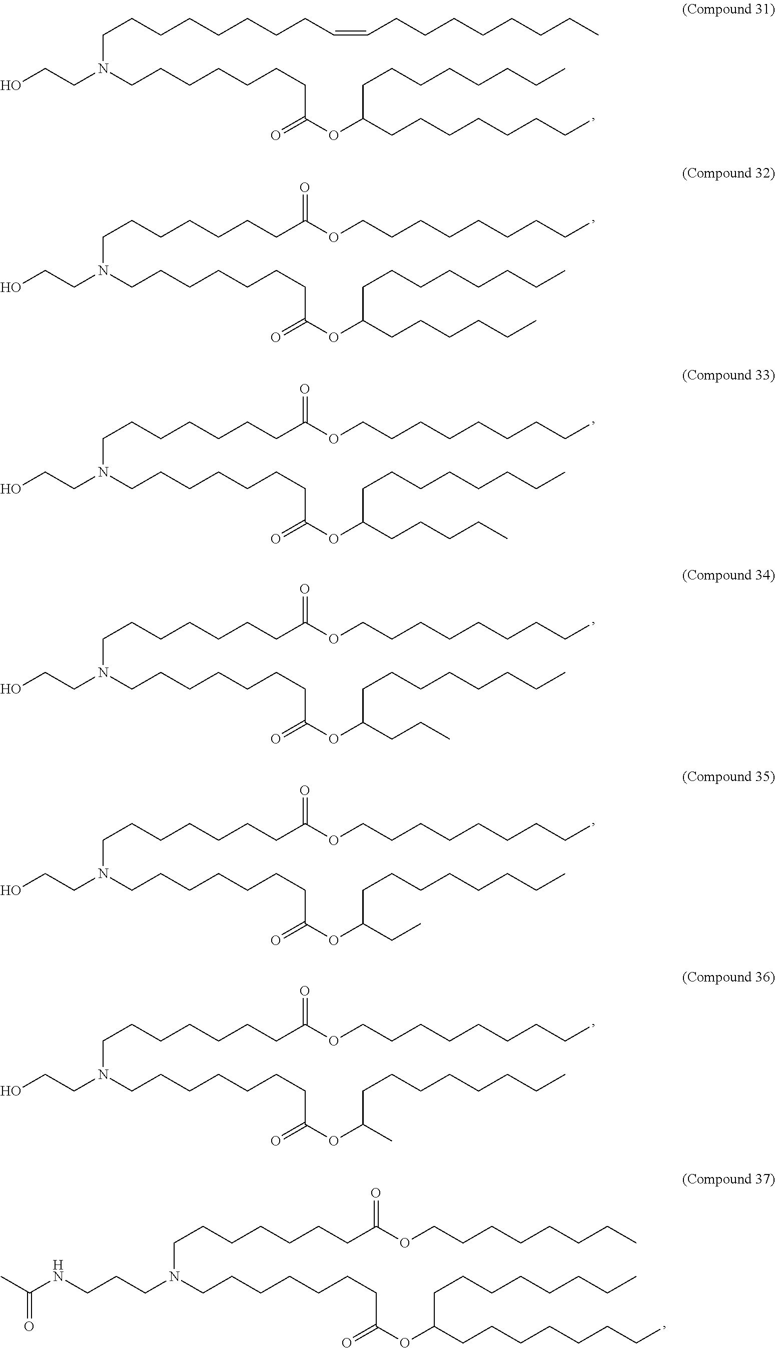

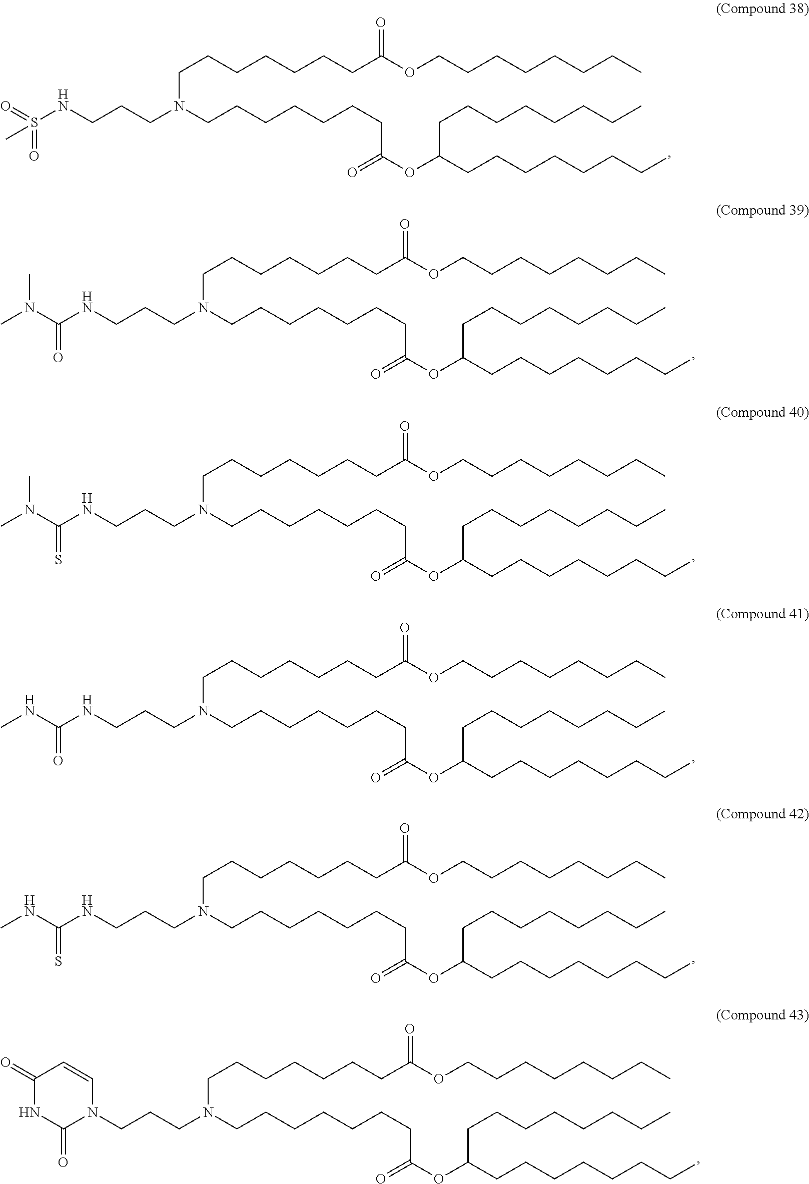

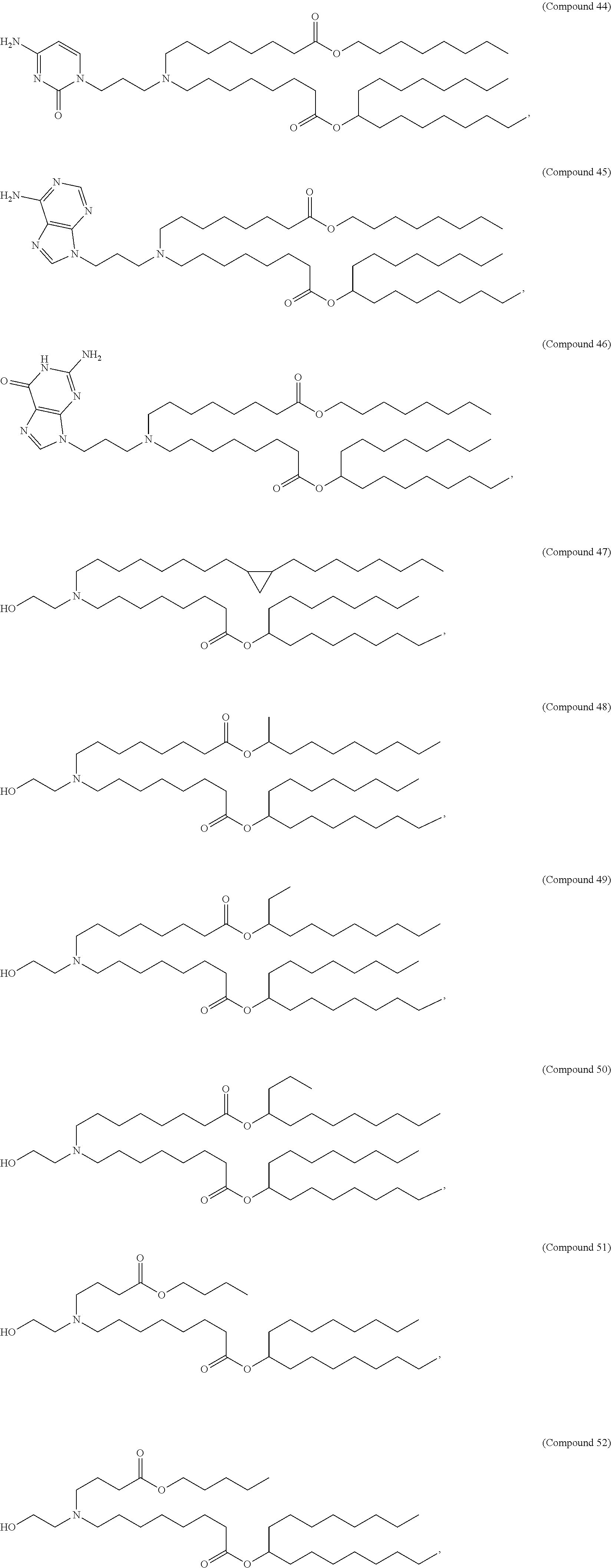

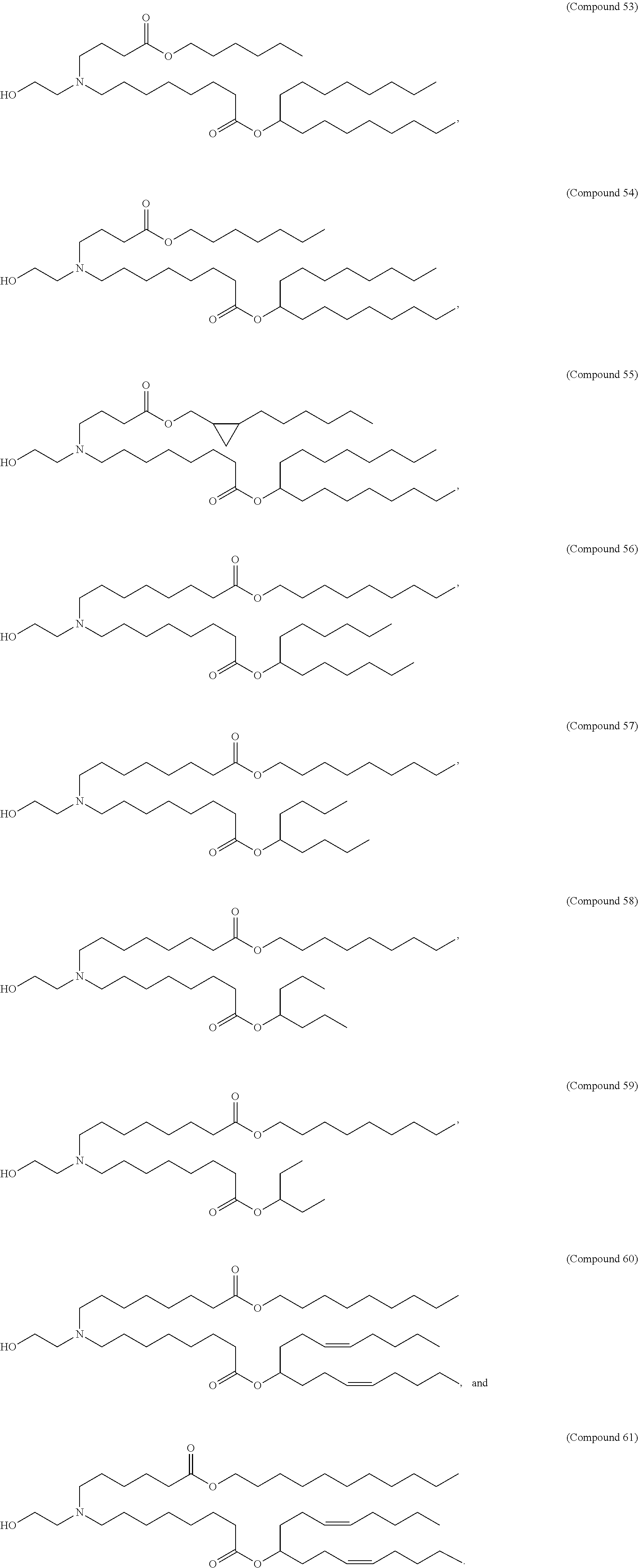

In further embodiments, the compound of Formula (I) is selected from the group consisting of:

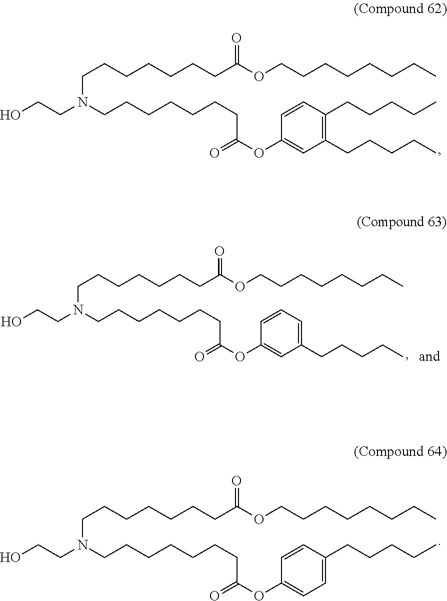 [SMILES: CCCCCCCCOC(=O)CCCCCCCN(CCO)CCCCCCCC(=O)Oc1ccc(CCCCC)c(CCCCC)c1 ; CCCCCCCCOC(=O)CCCCCCCN(CCO)CCCCCCCC(=O)Oc1cccc(CCCCC)c1 ; CCCCCCCCOC(=O)CCCCCCCN(CCO)CCCCCCCC(=O)Oc1ccc(CCCCC)cc1]

In some embodiments, the compound of Formula (I) is selected from the group consisting of:

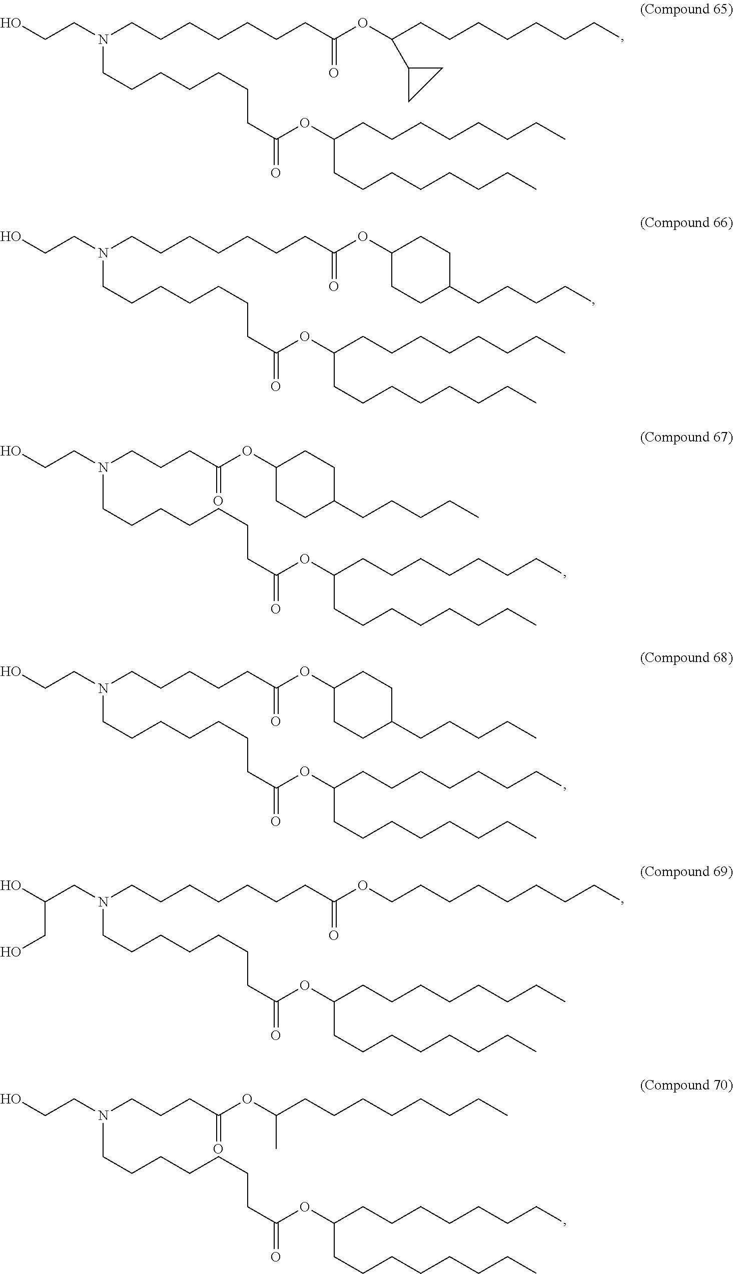

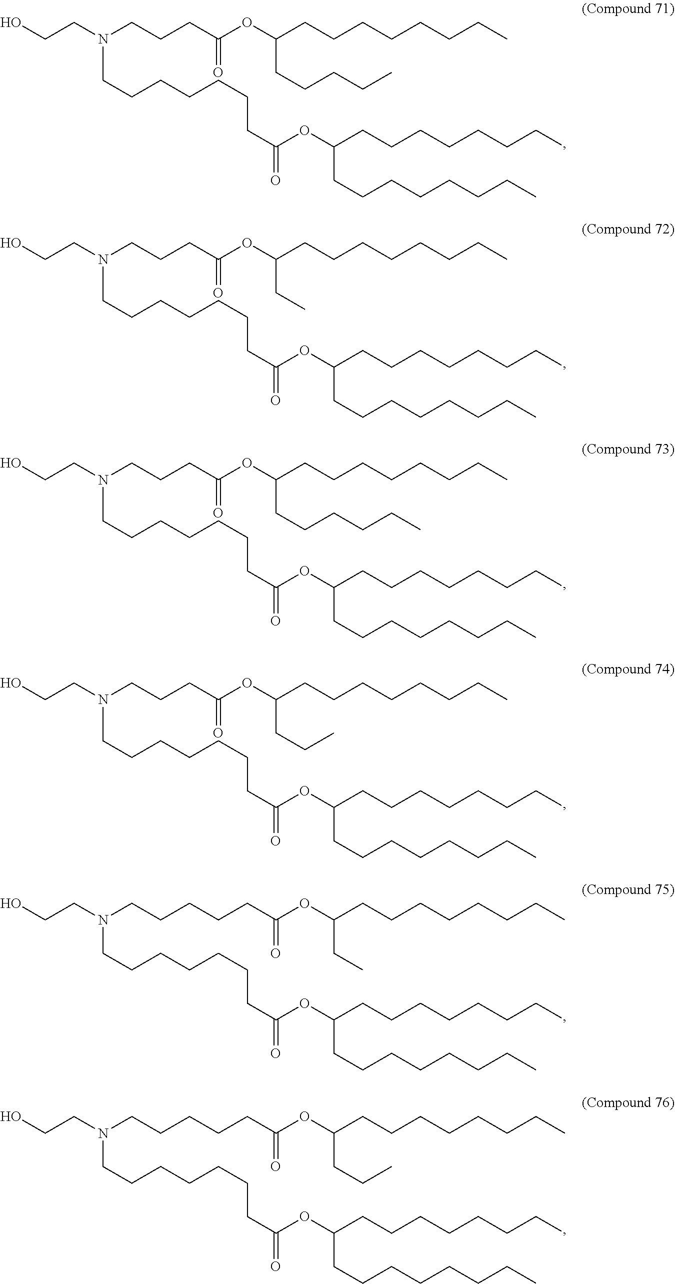

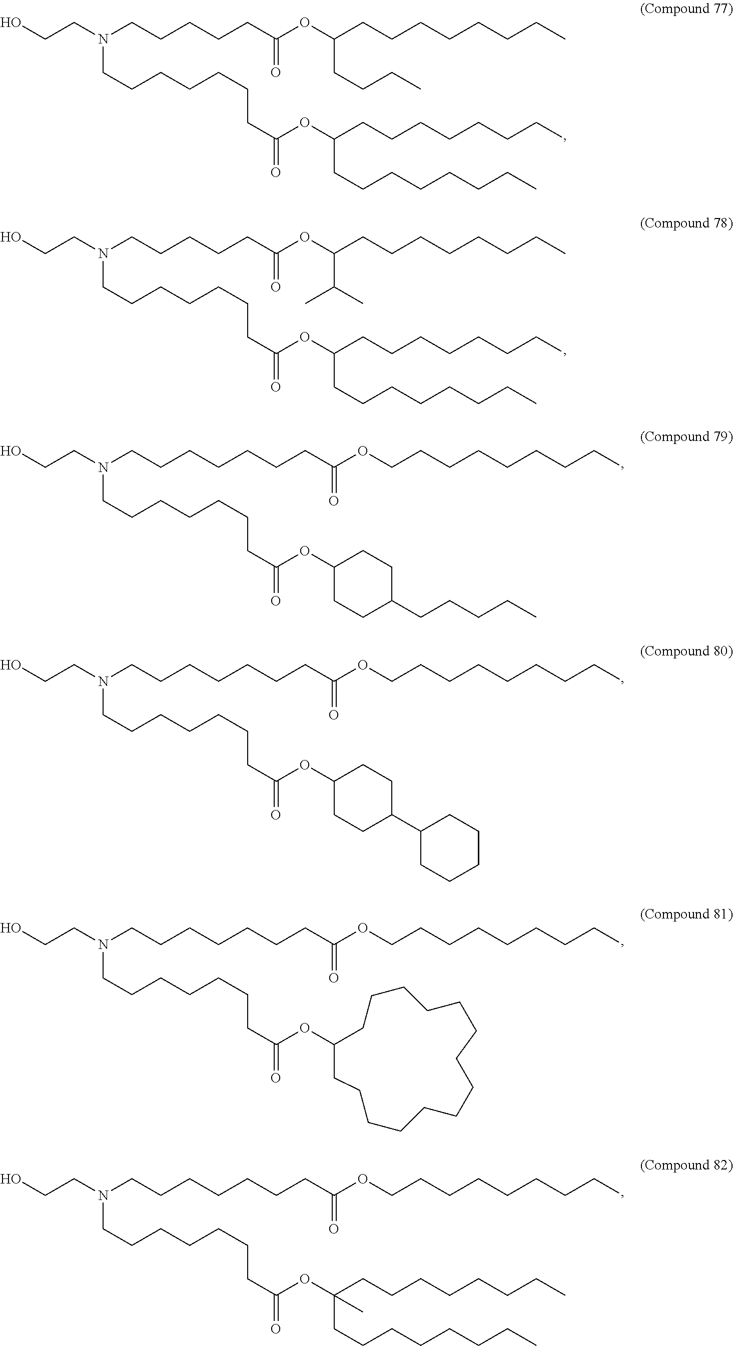

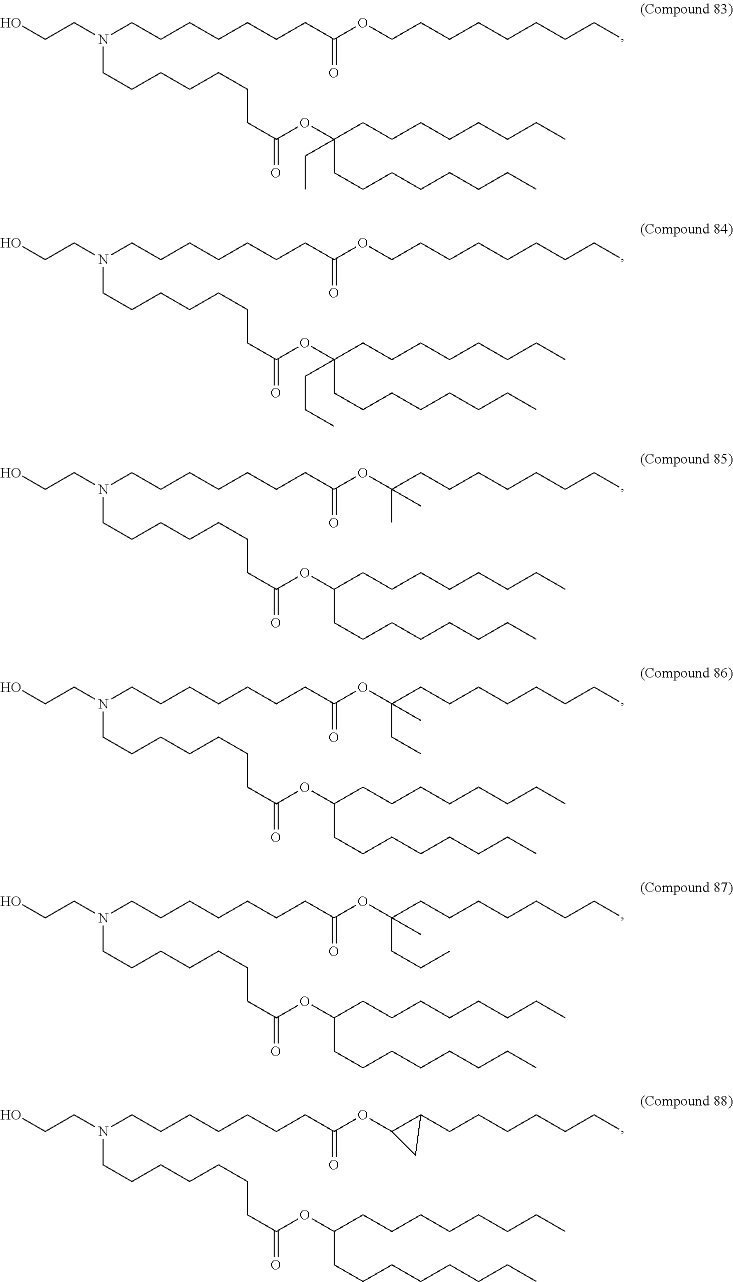

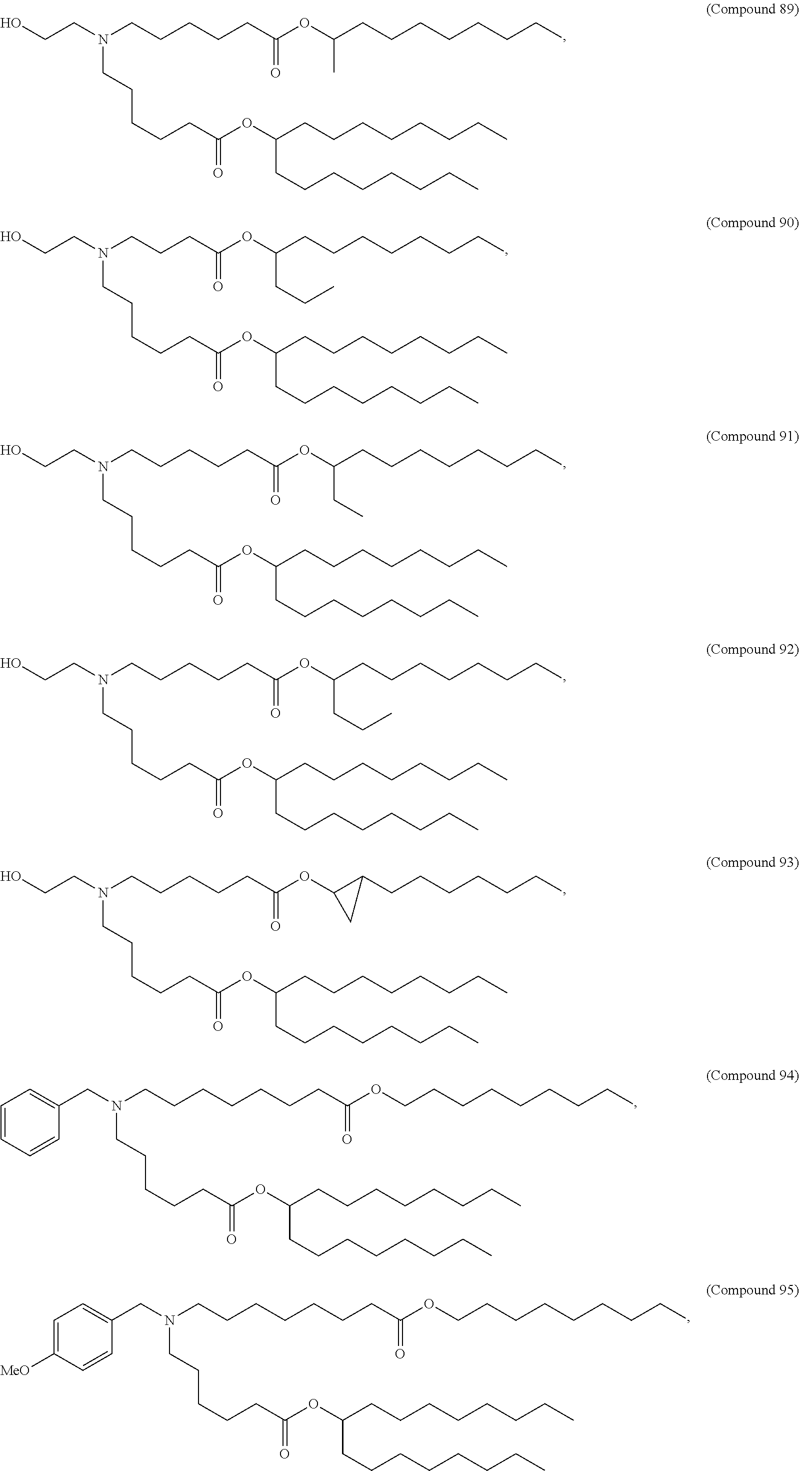

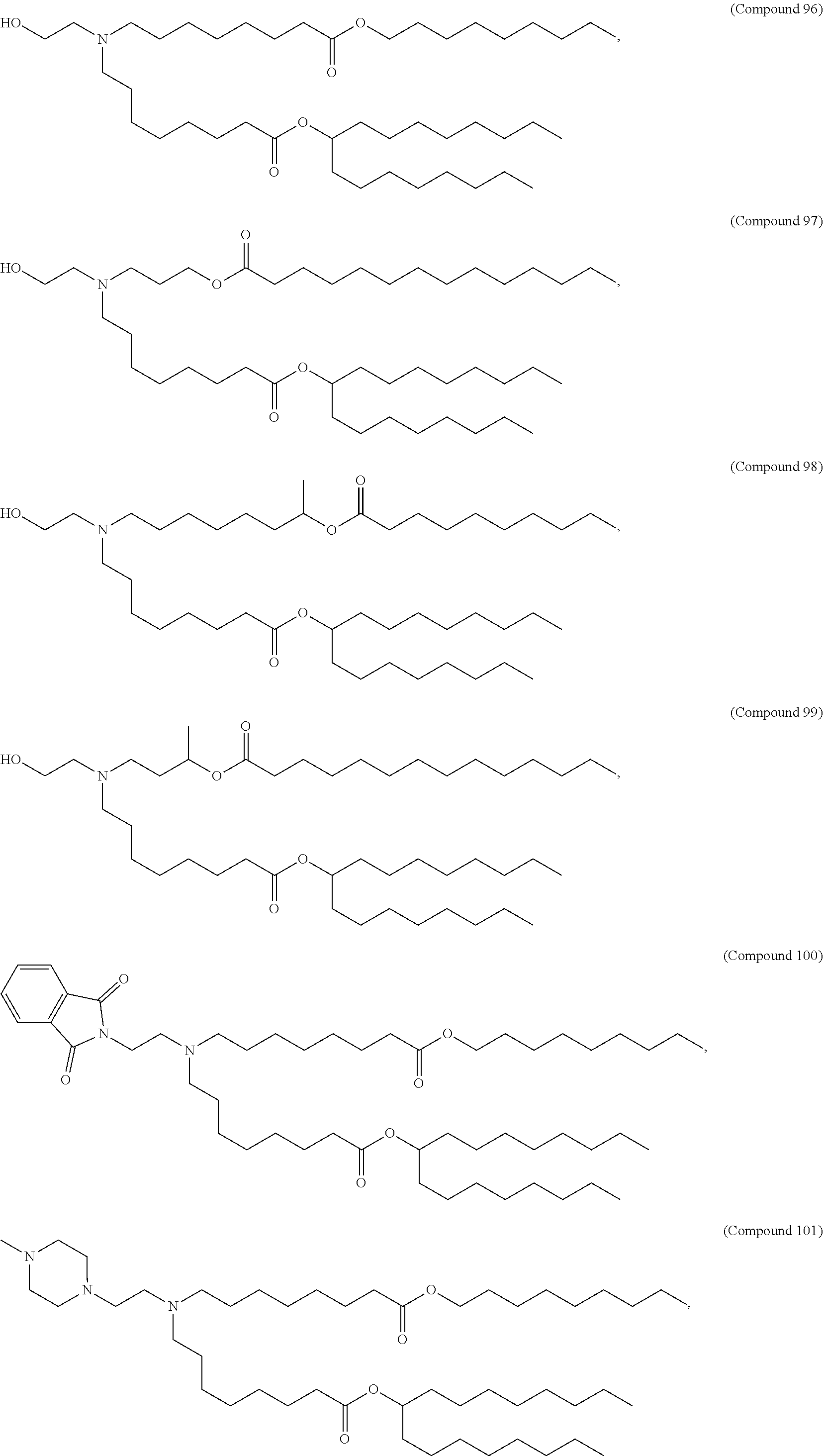

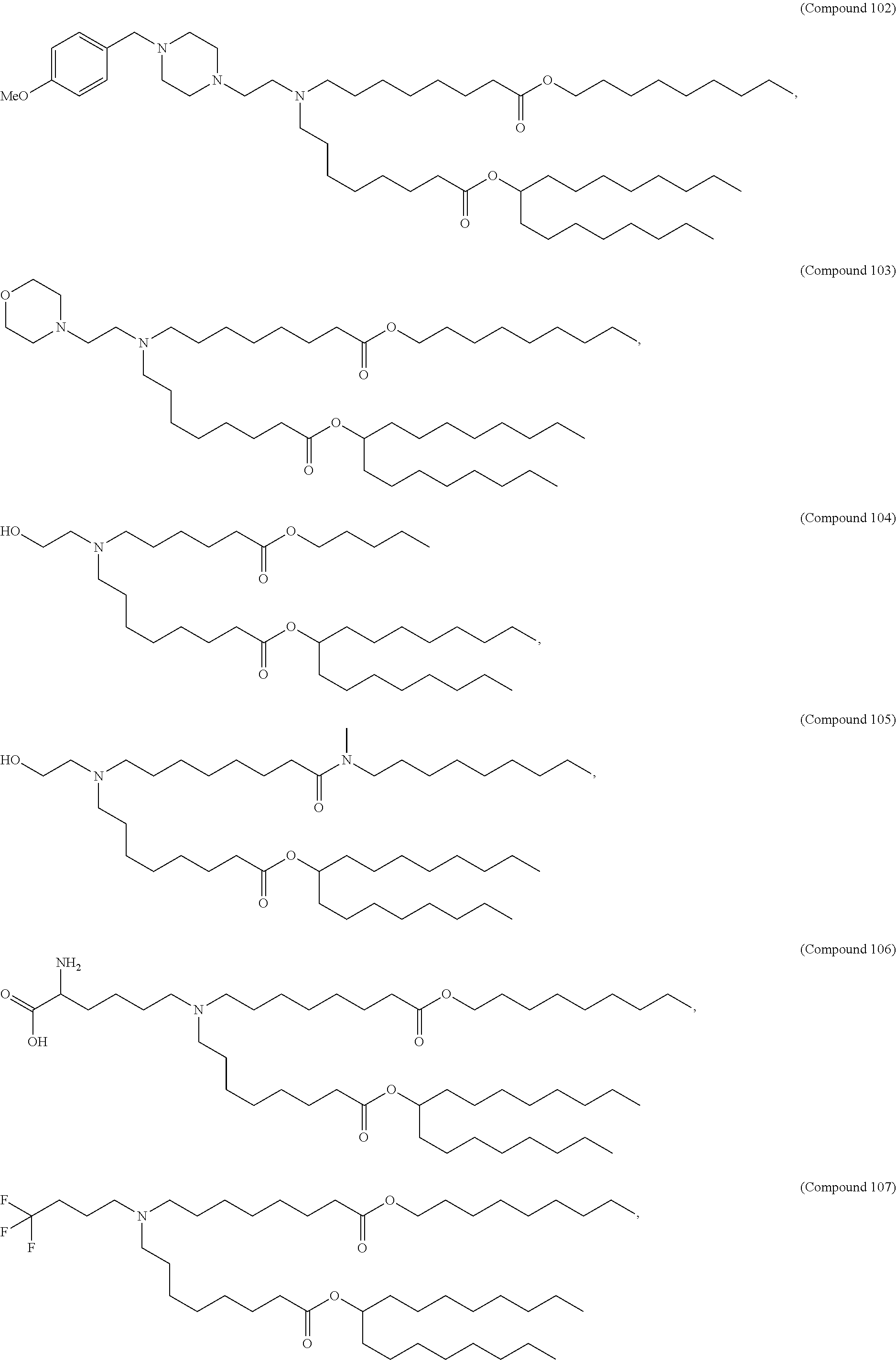

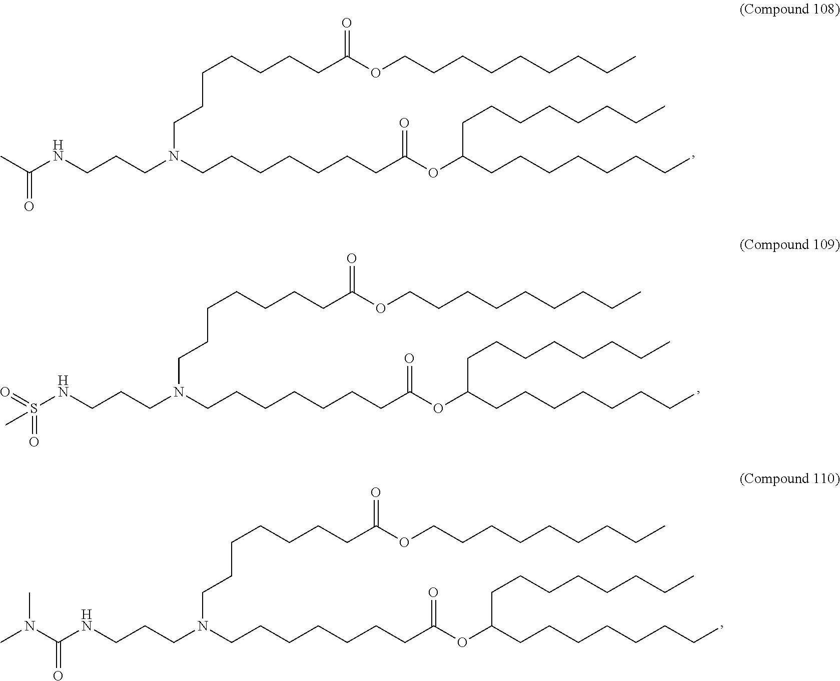

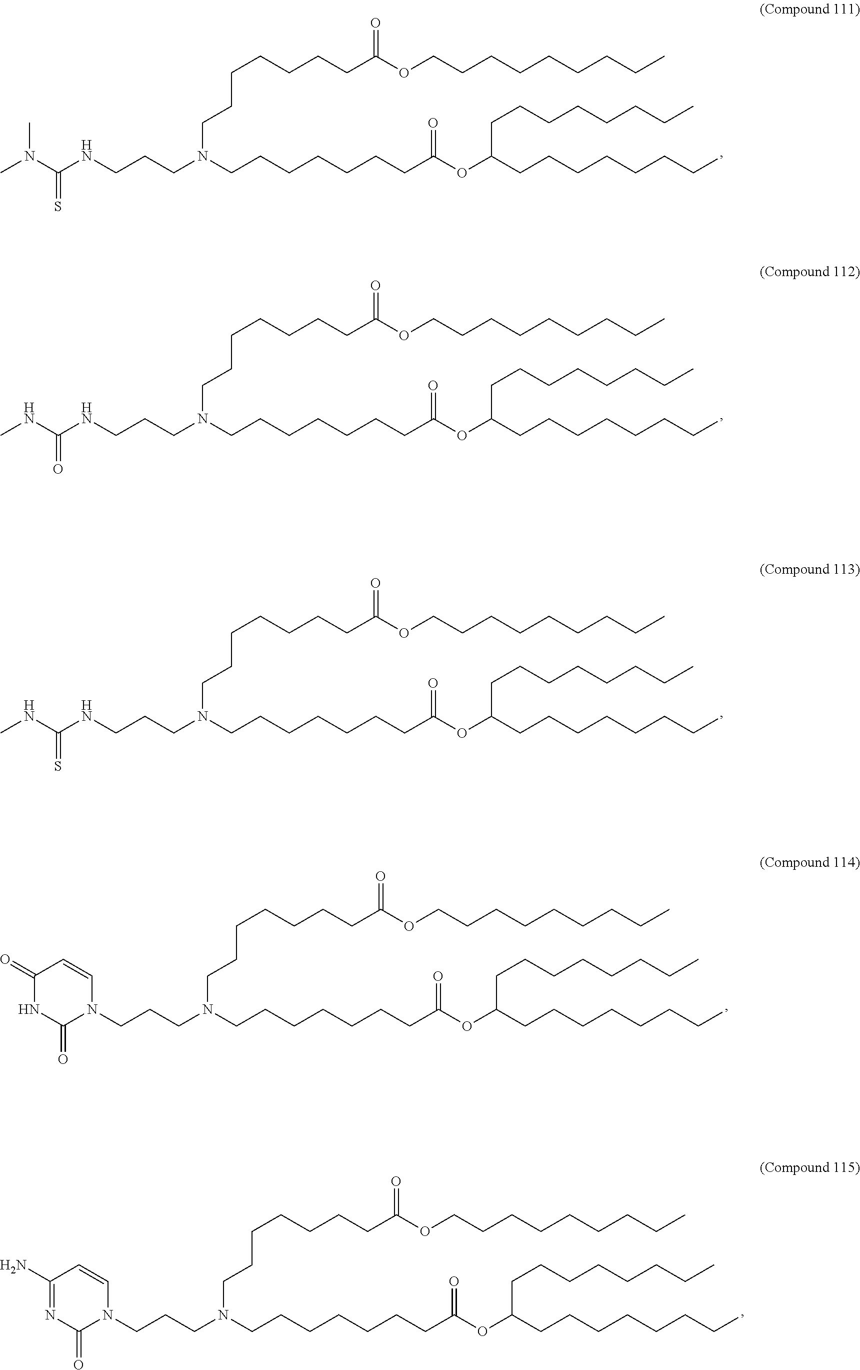

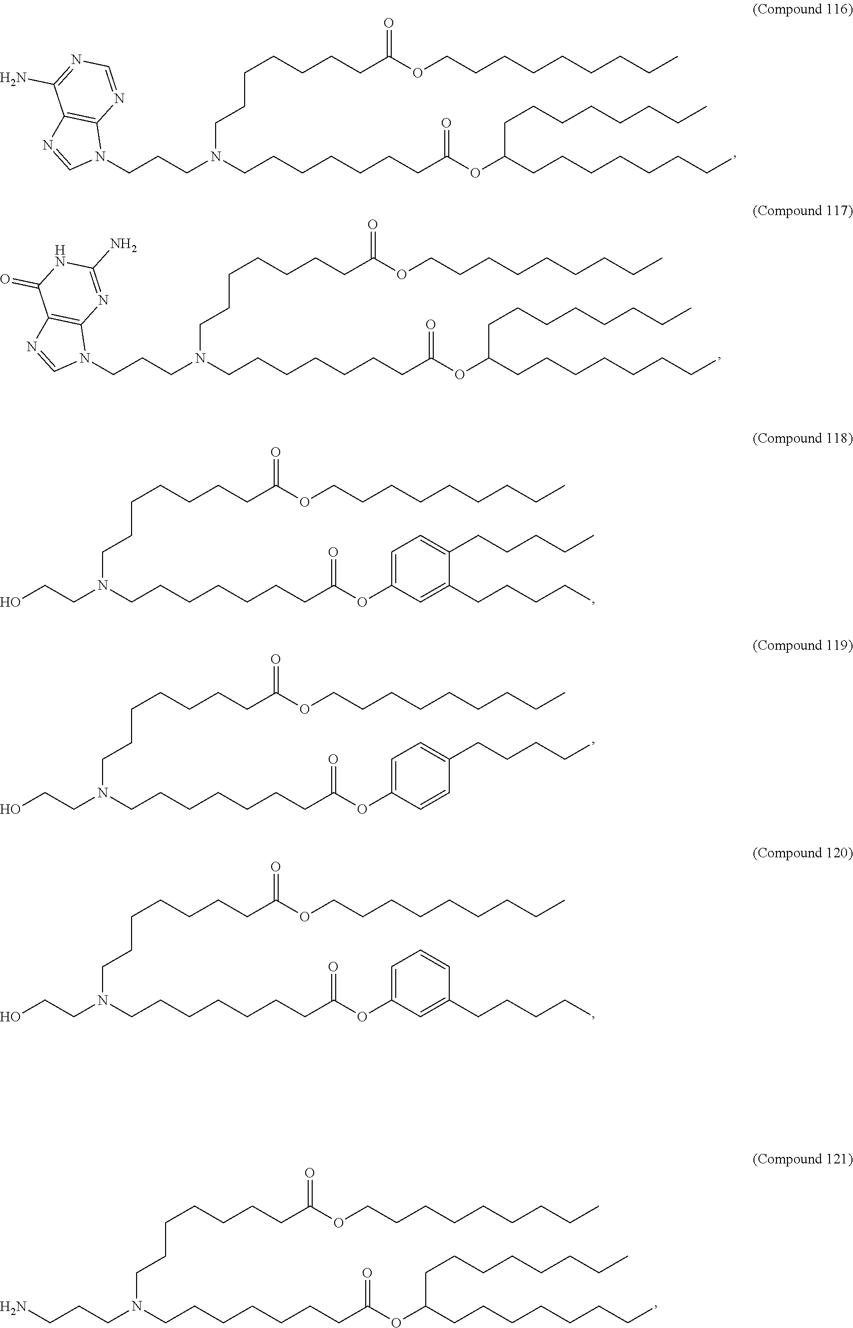

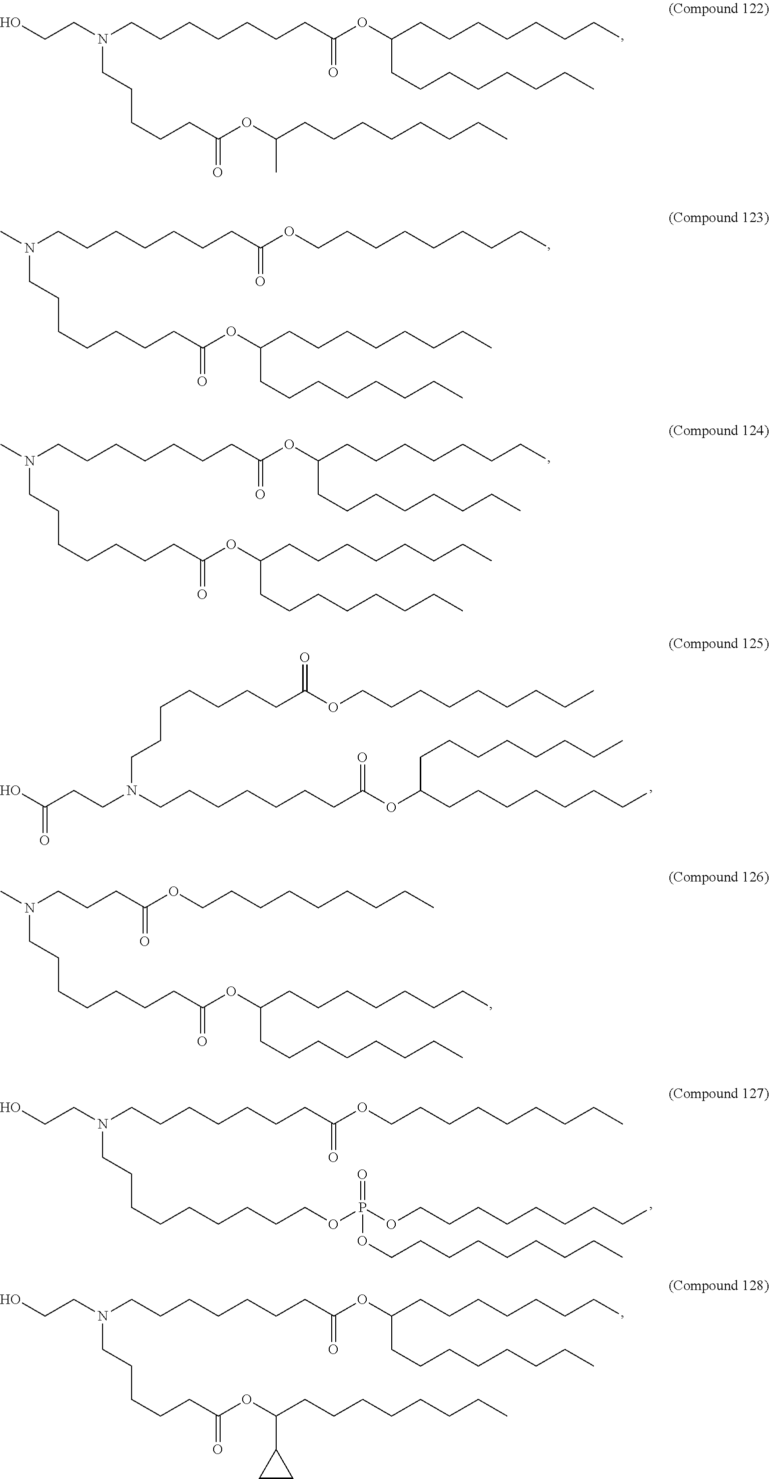

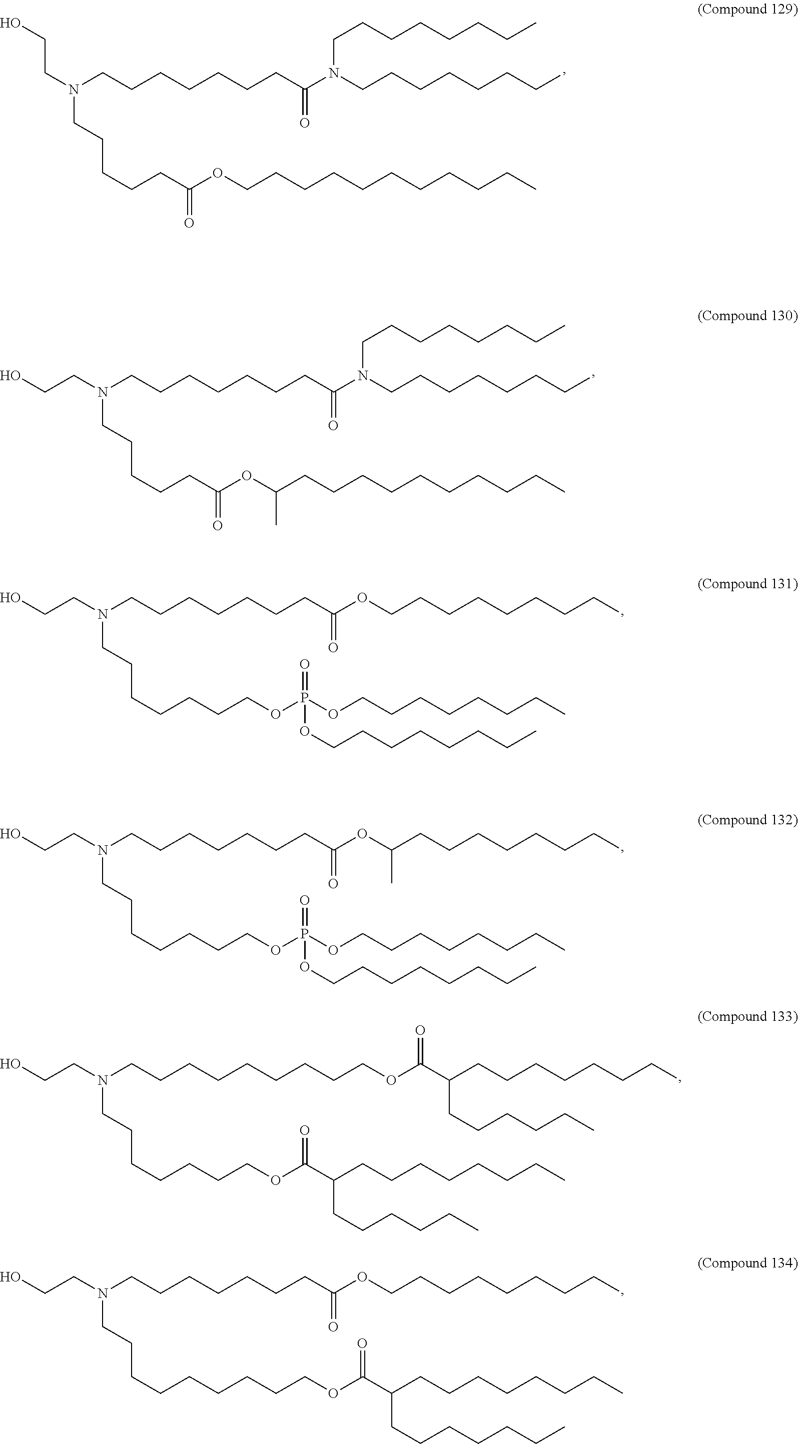

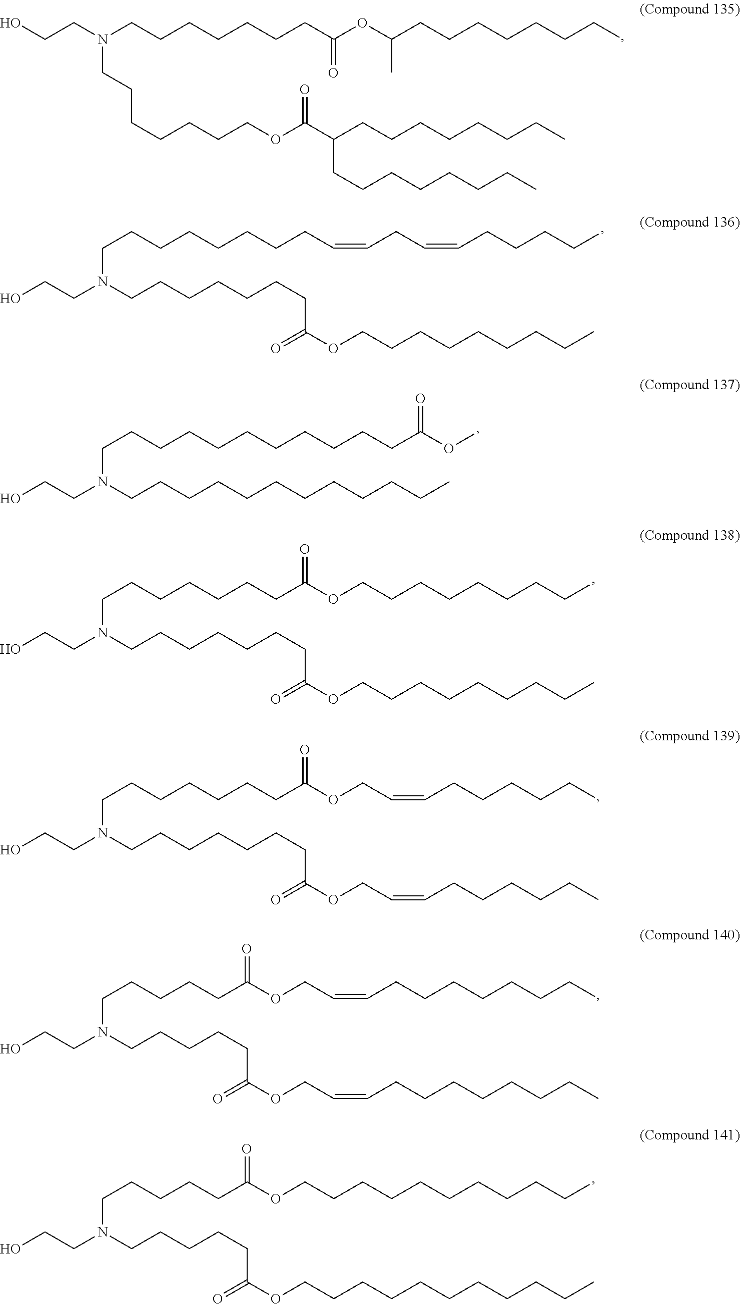

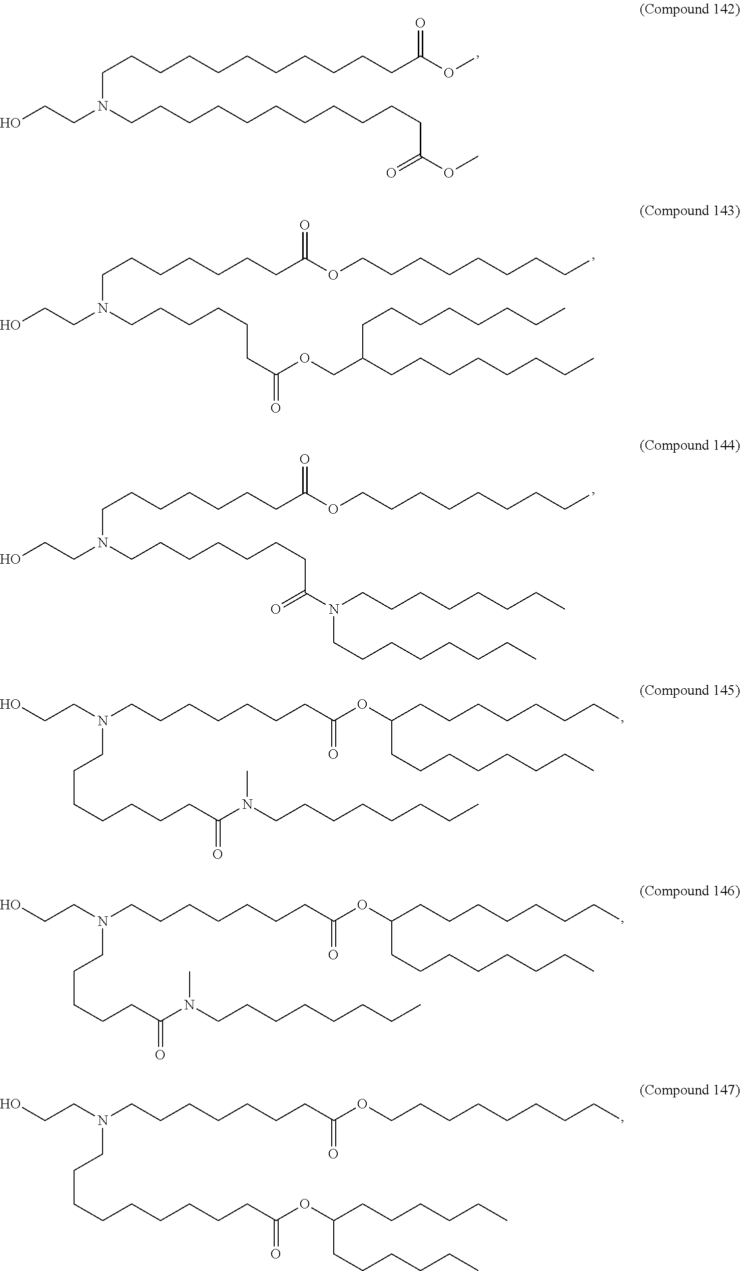

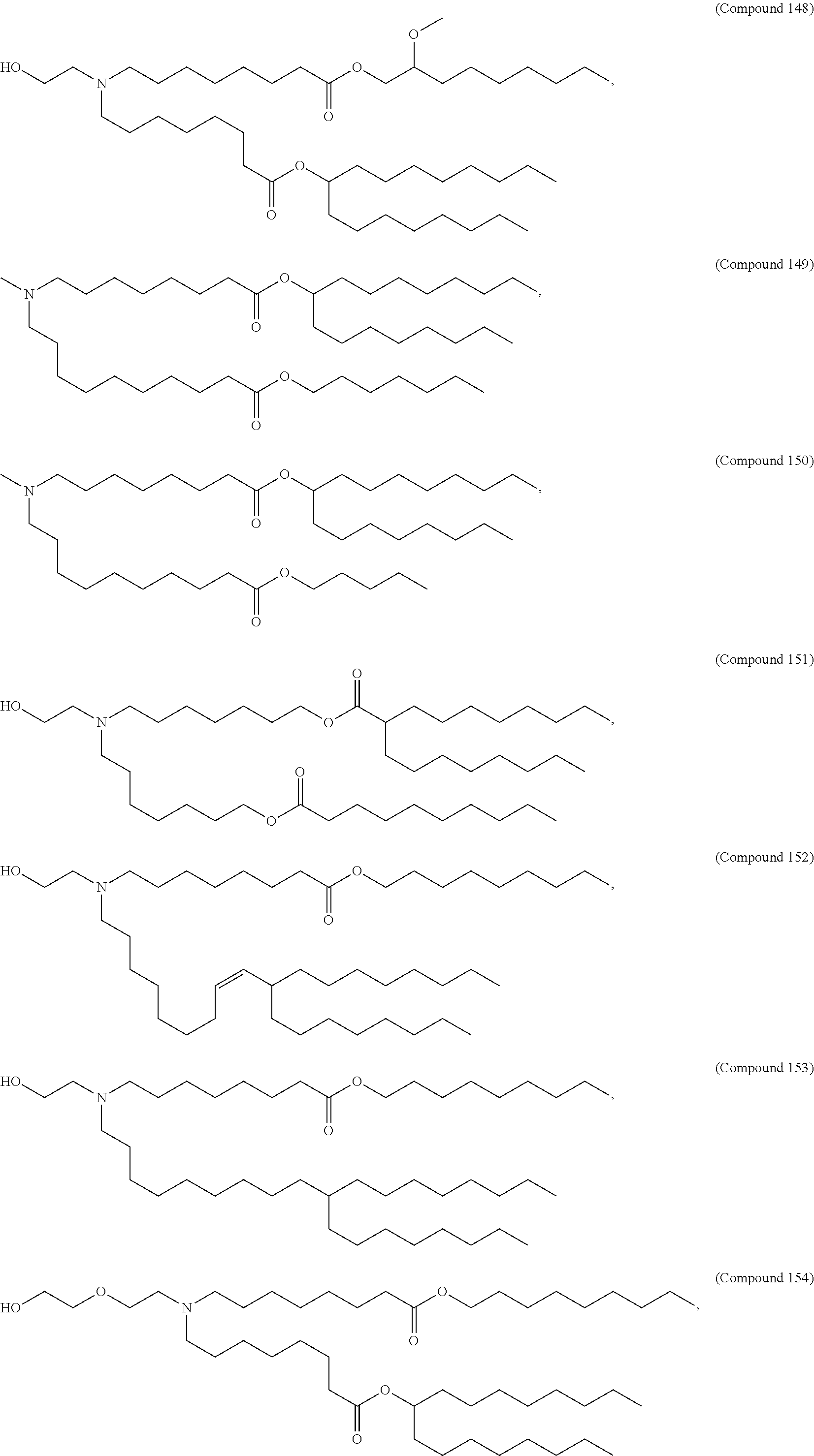

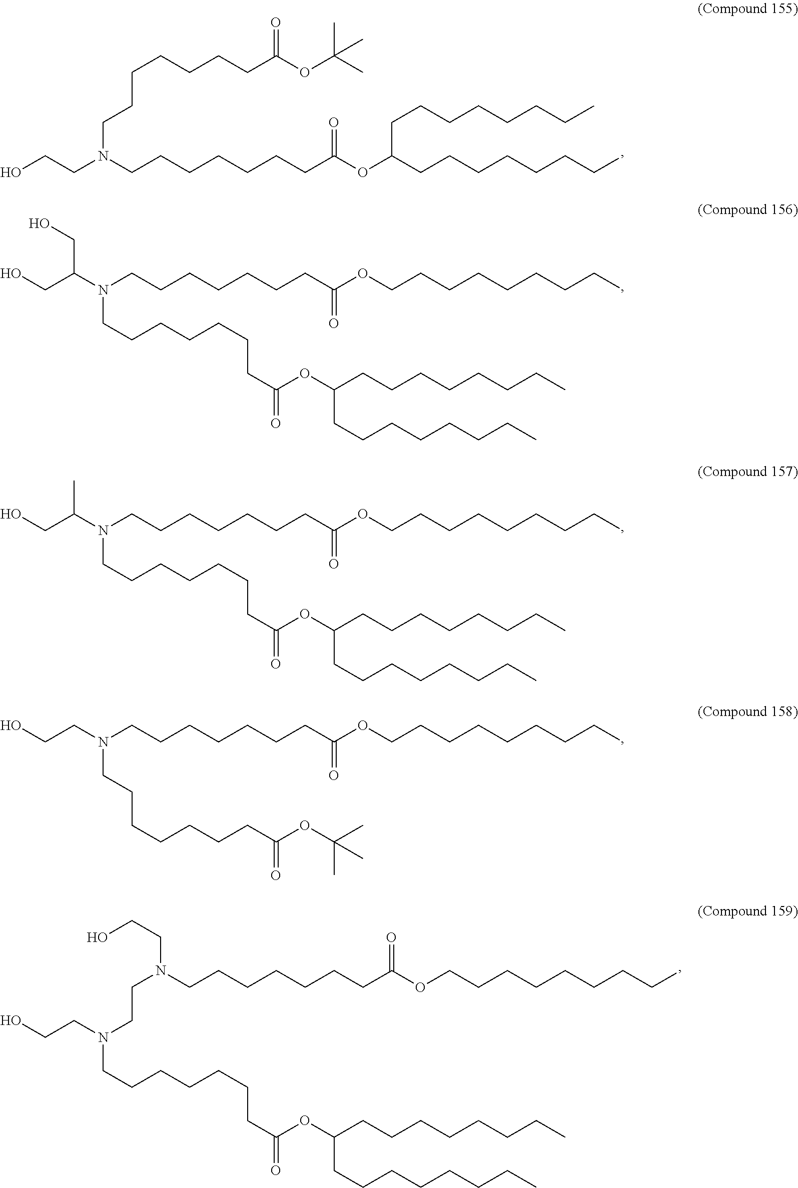

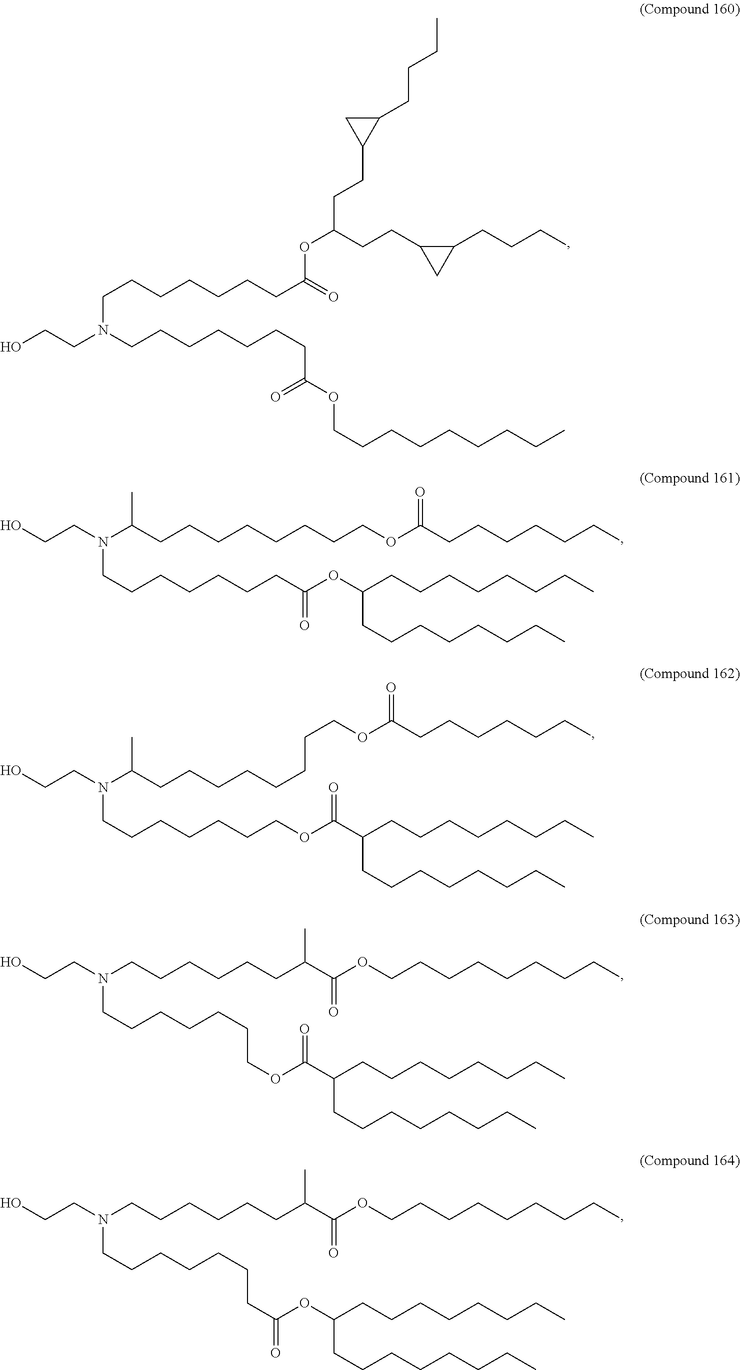

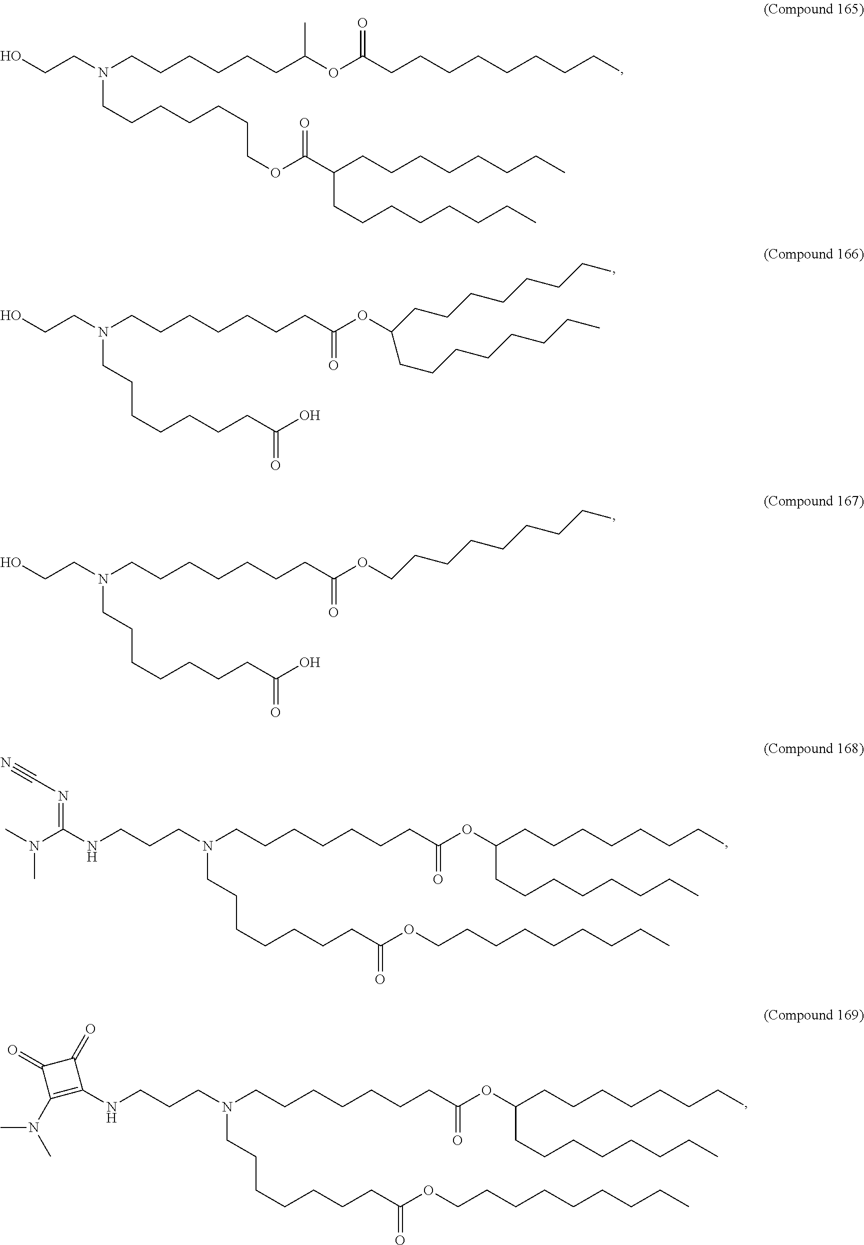

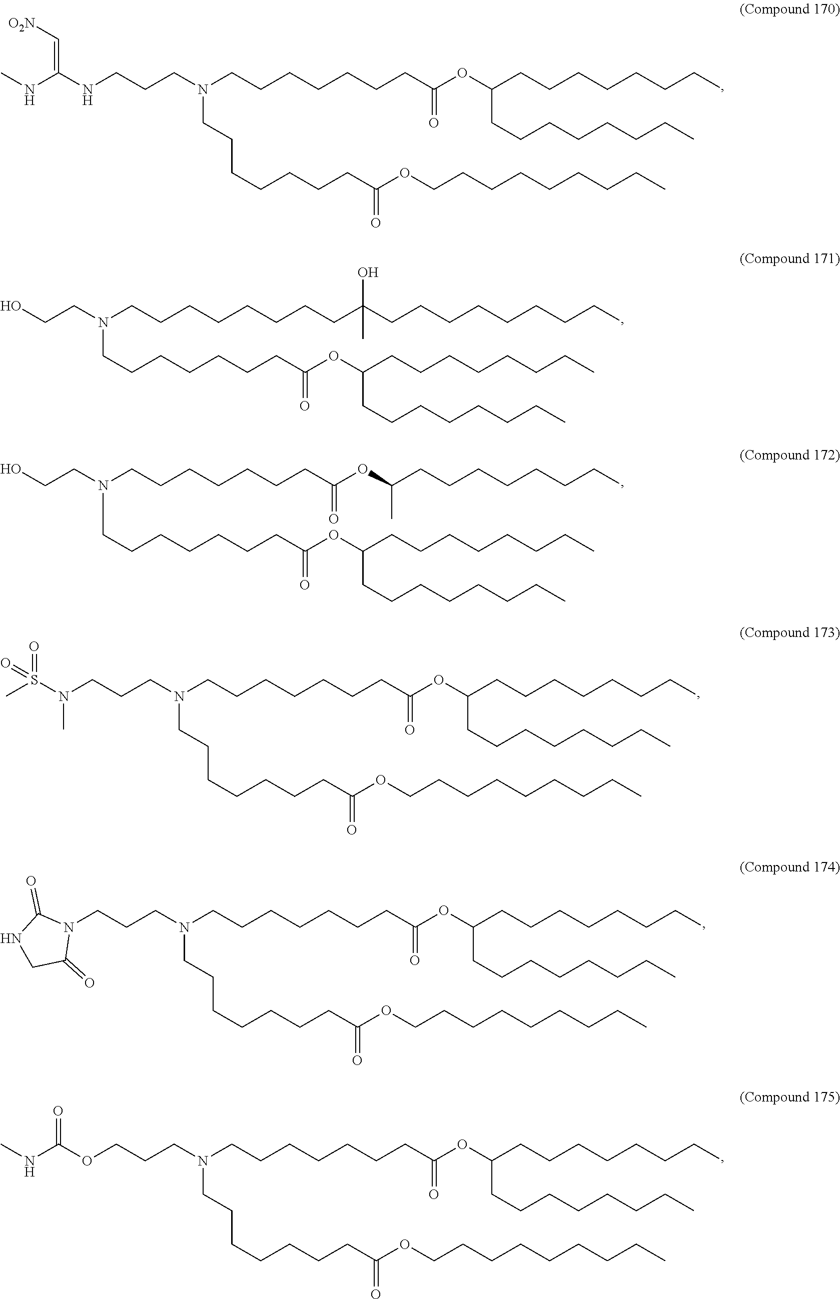

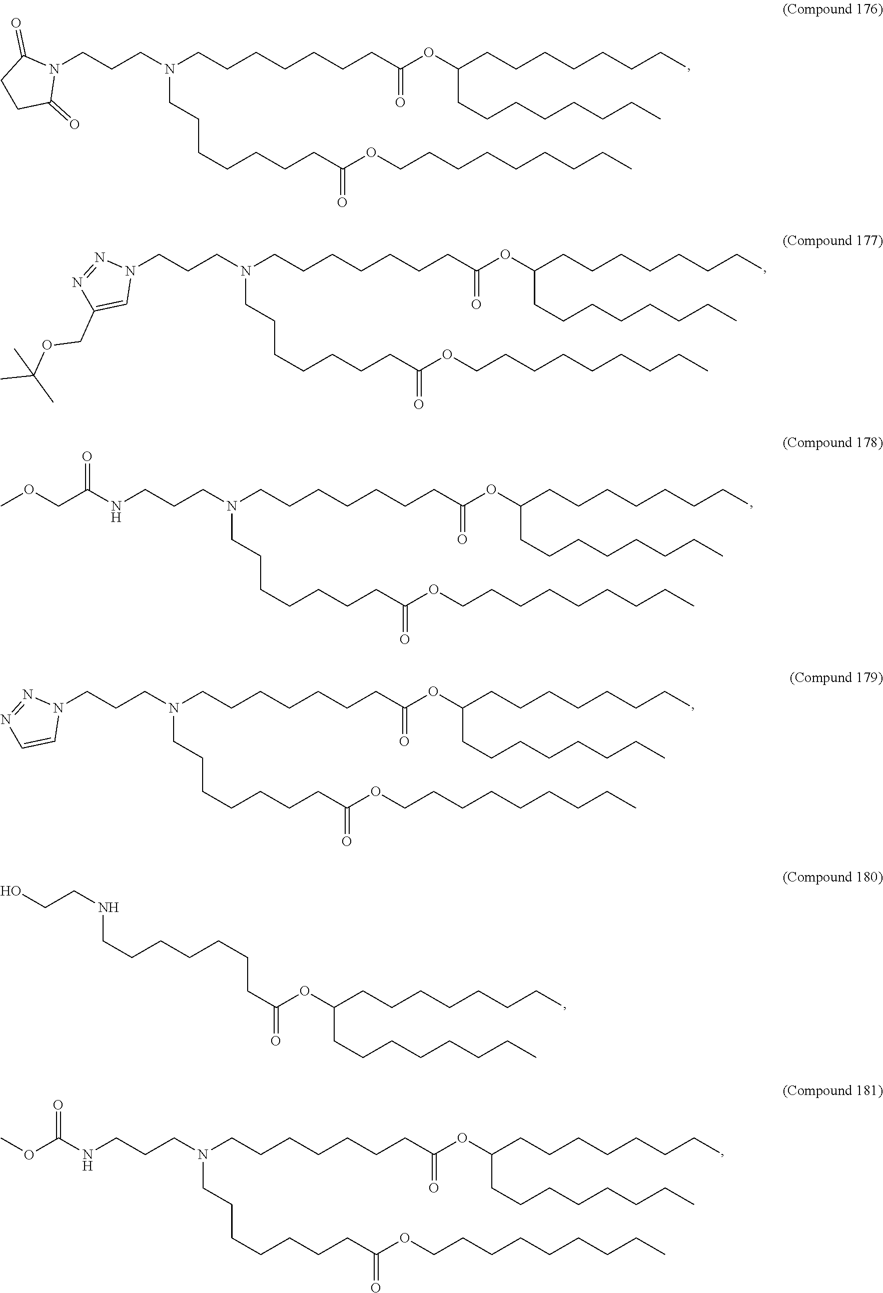

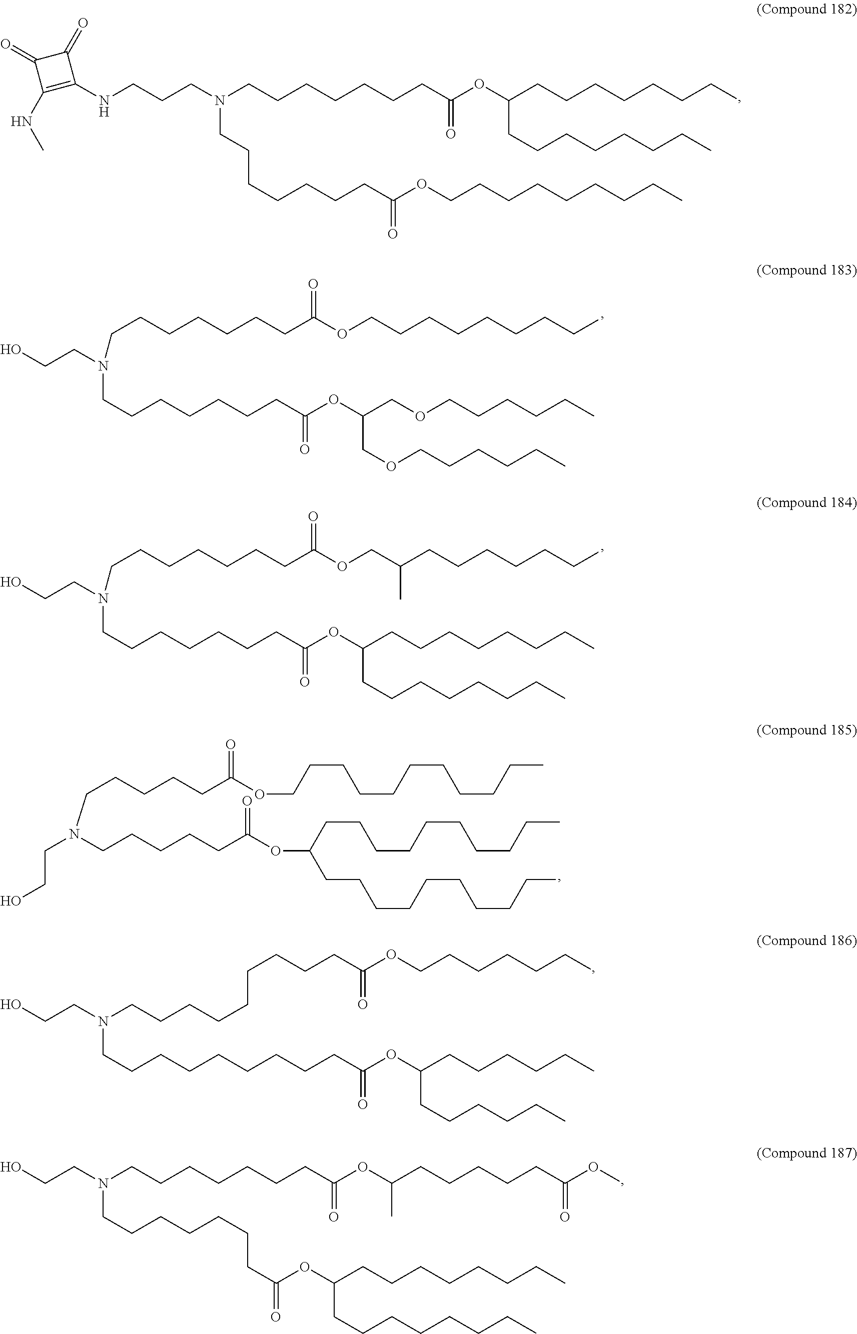

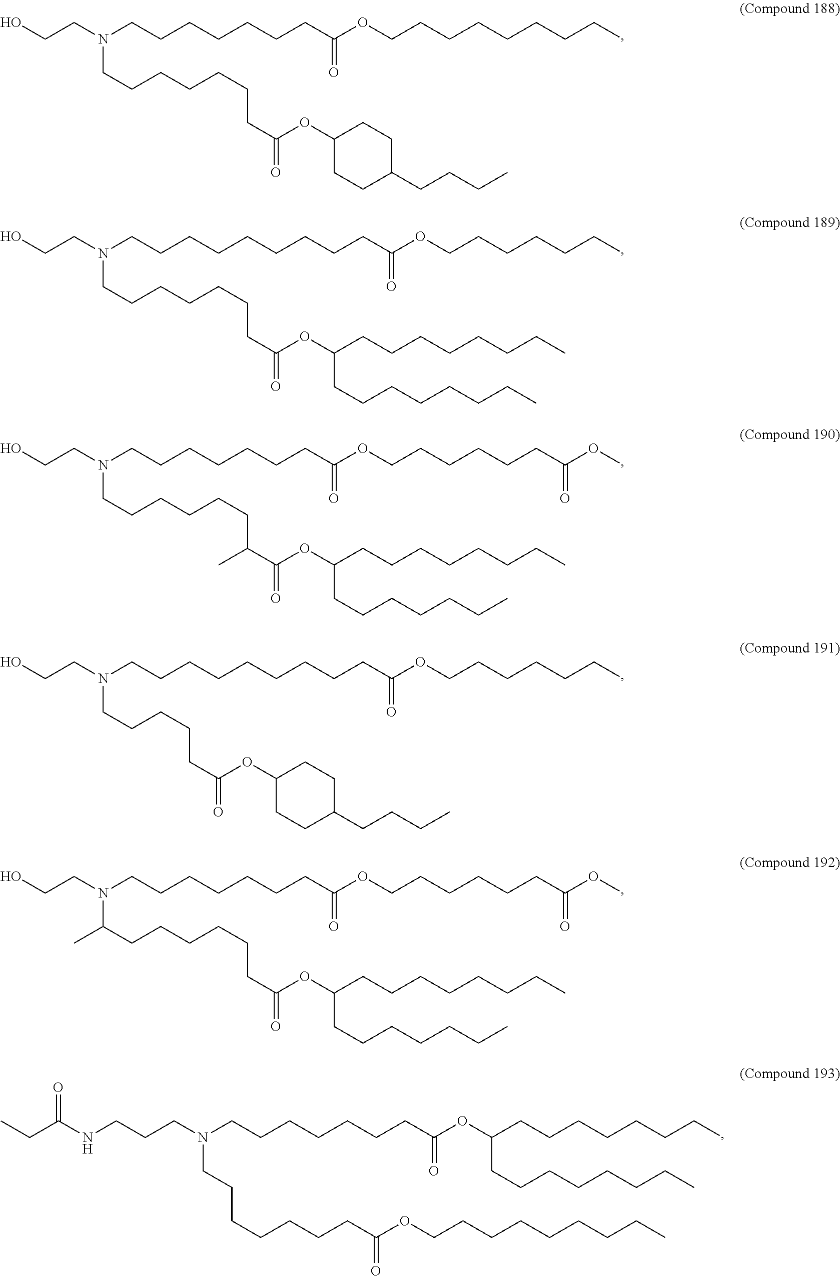

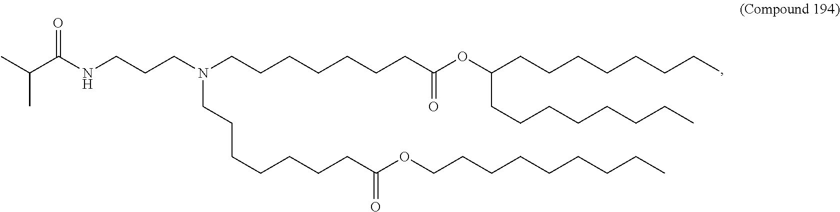

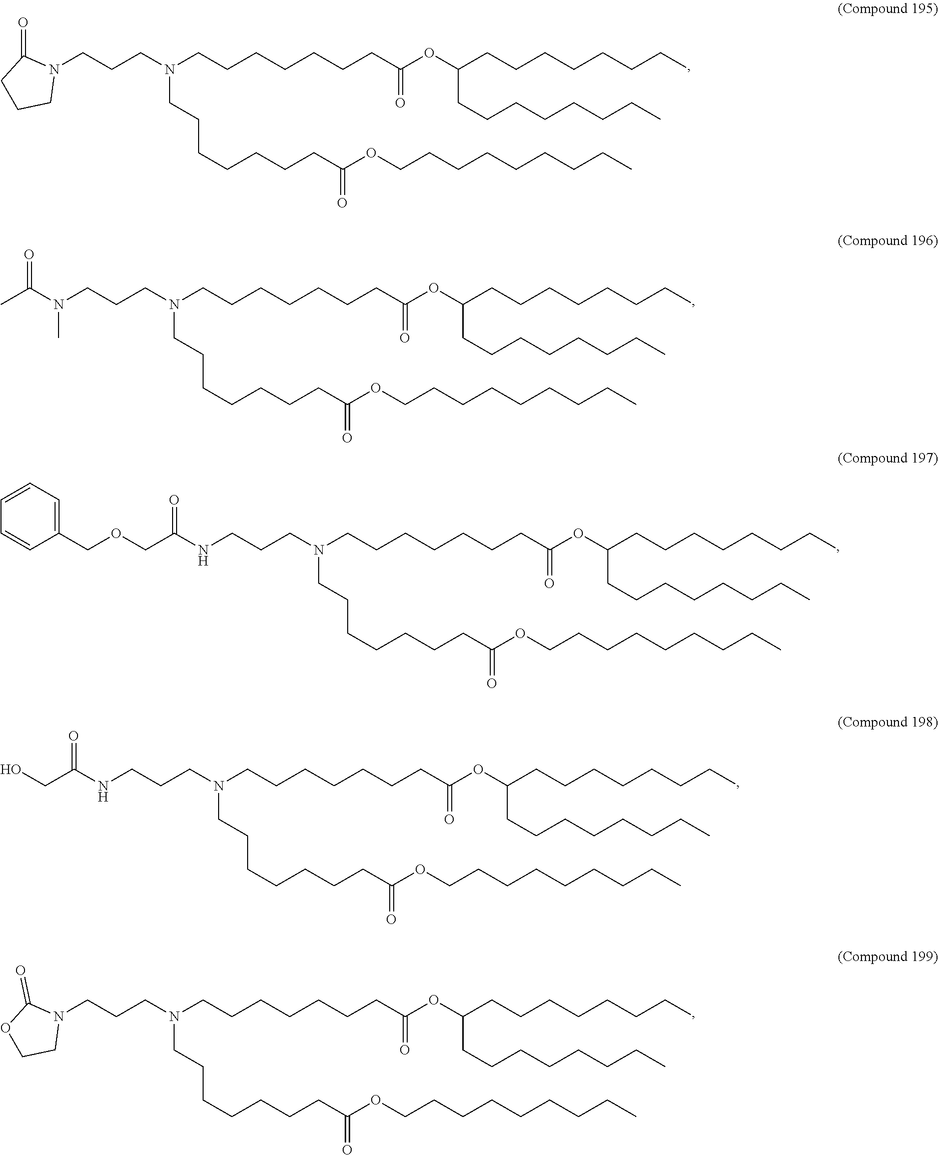

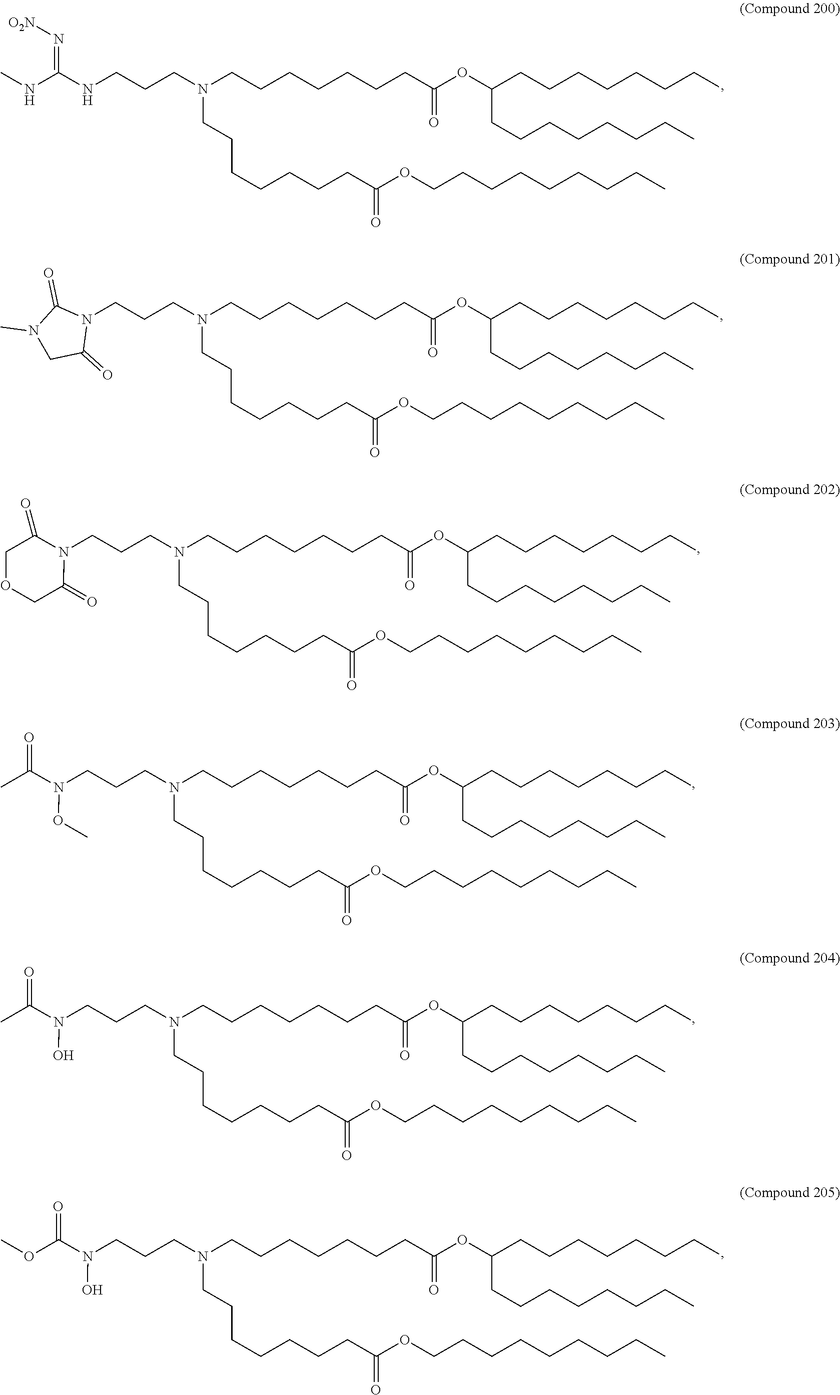

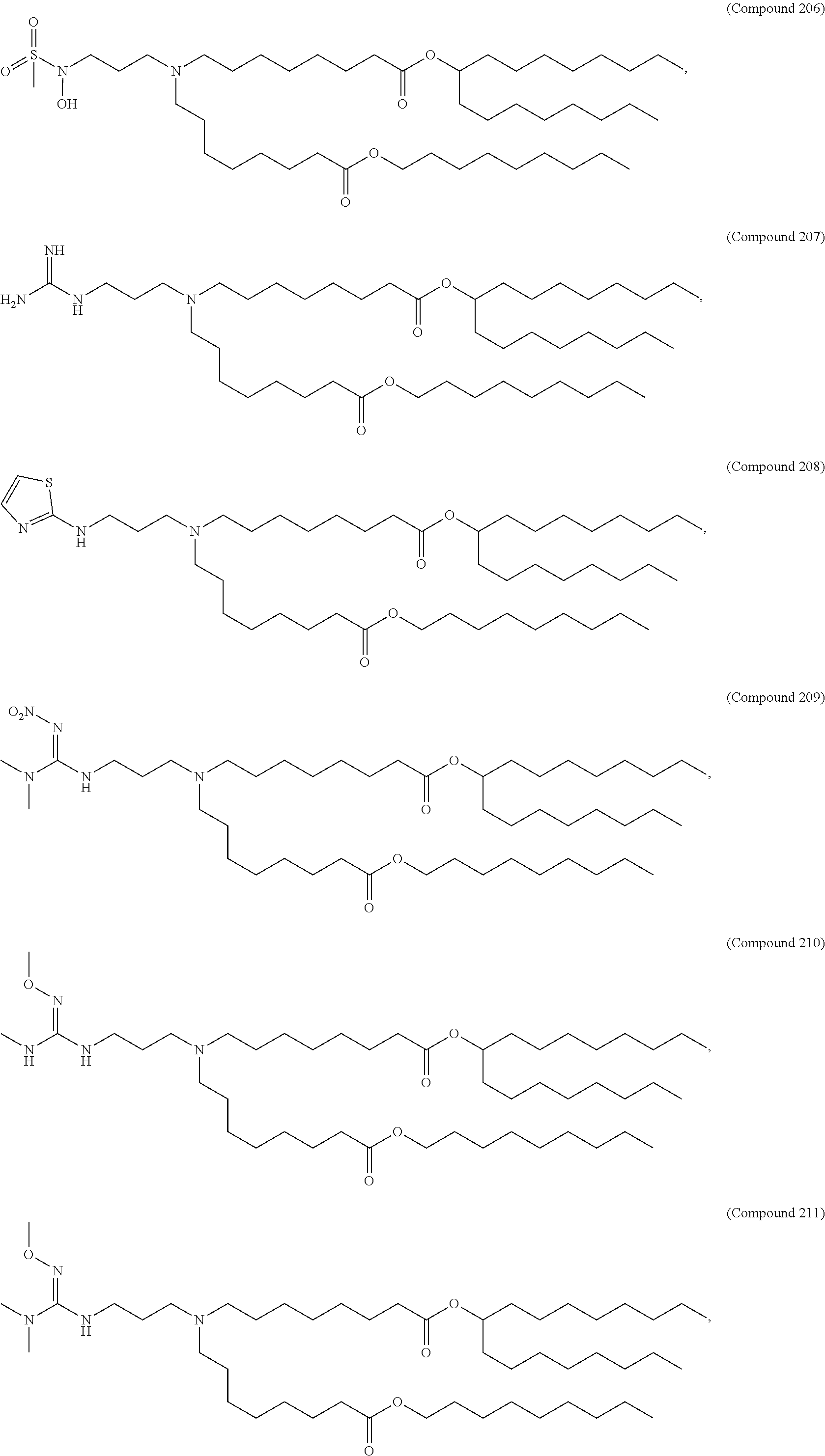

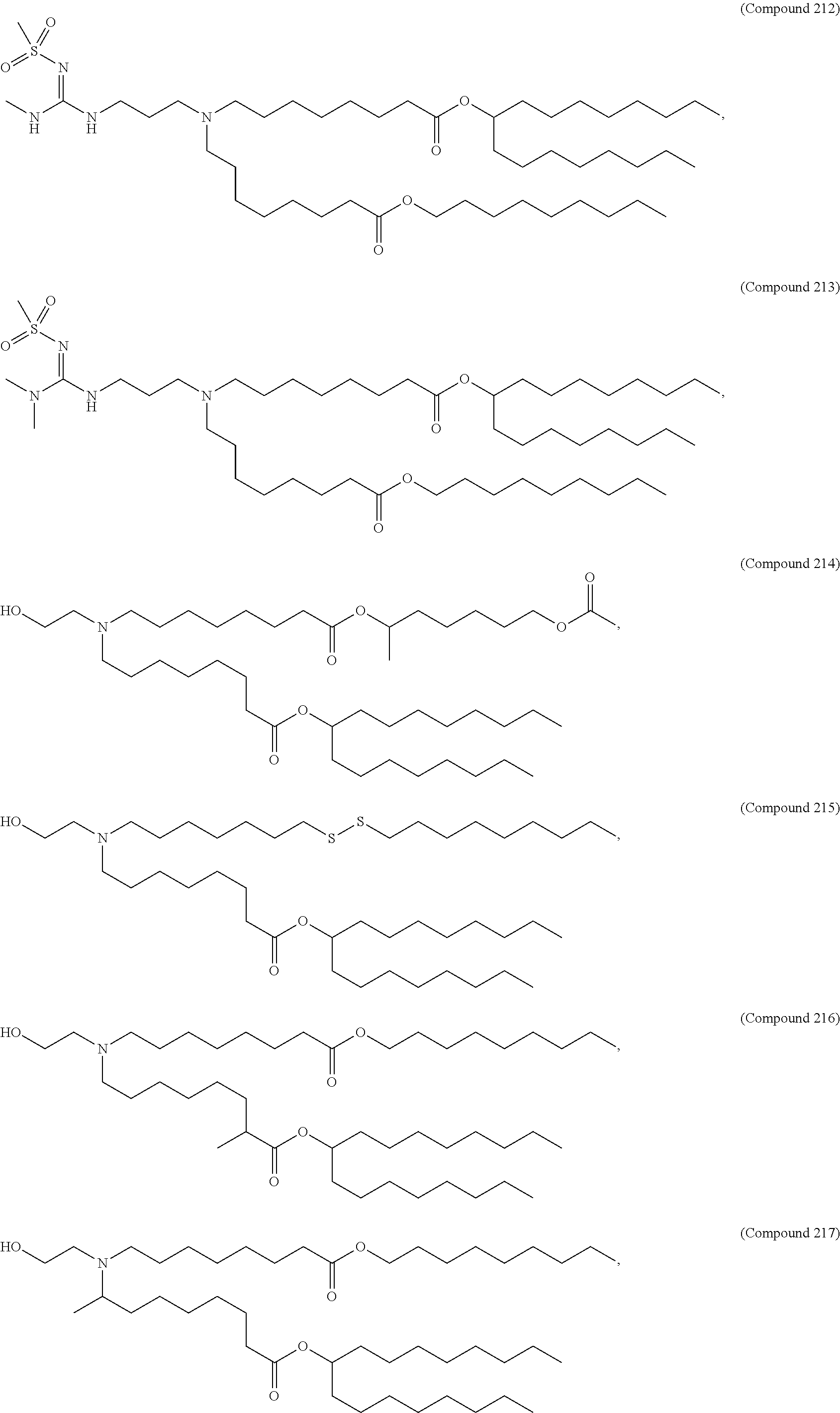

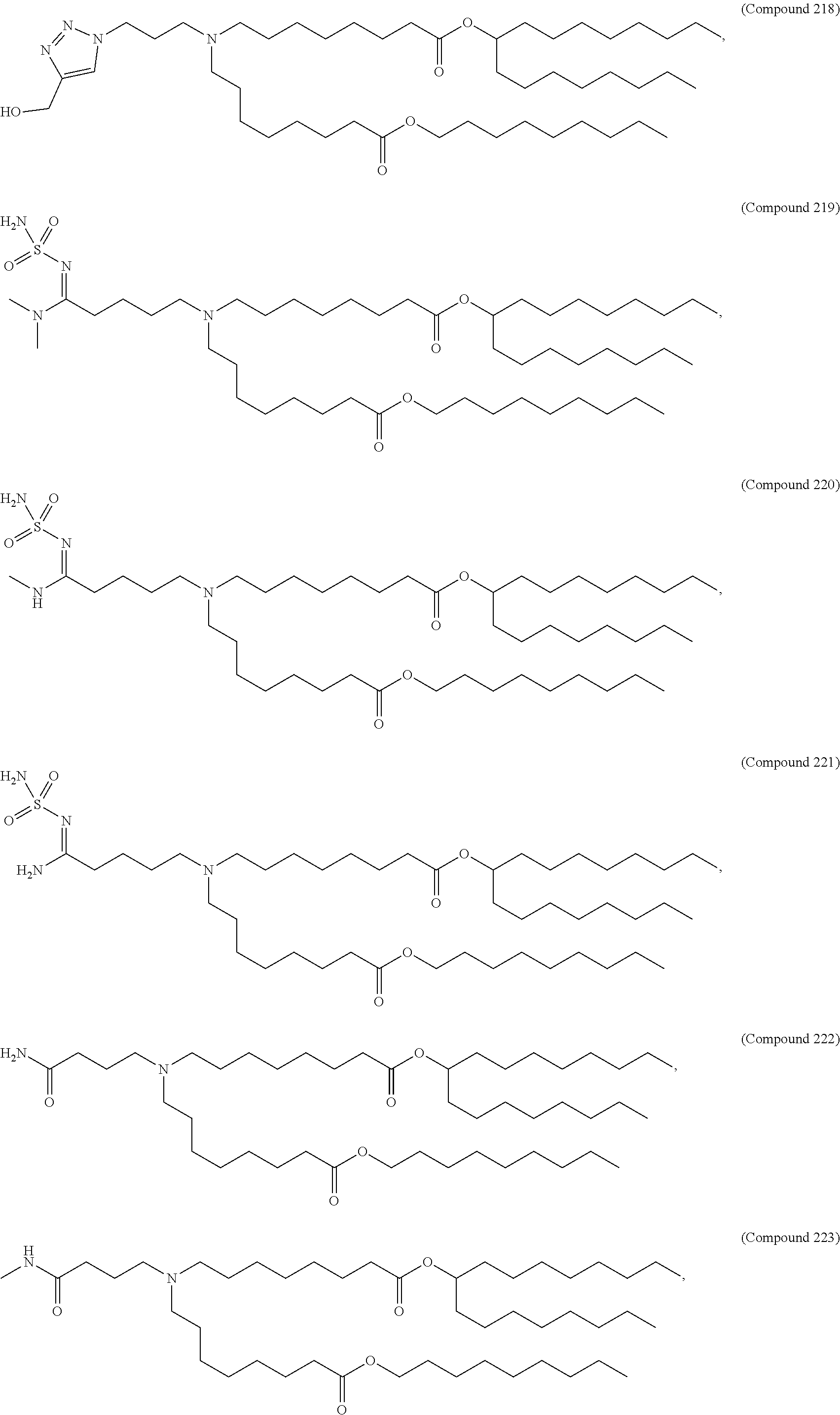

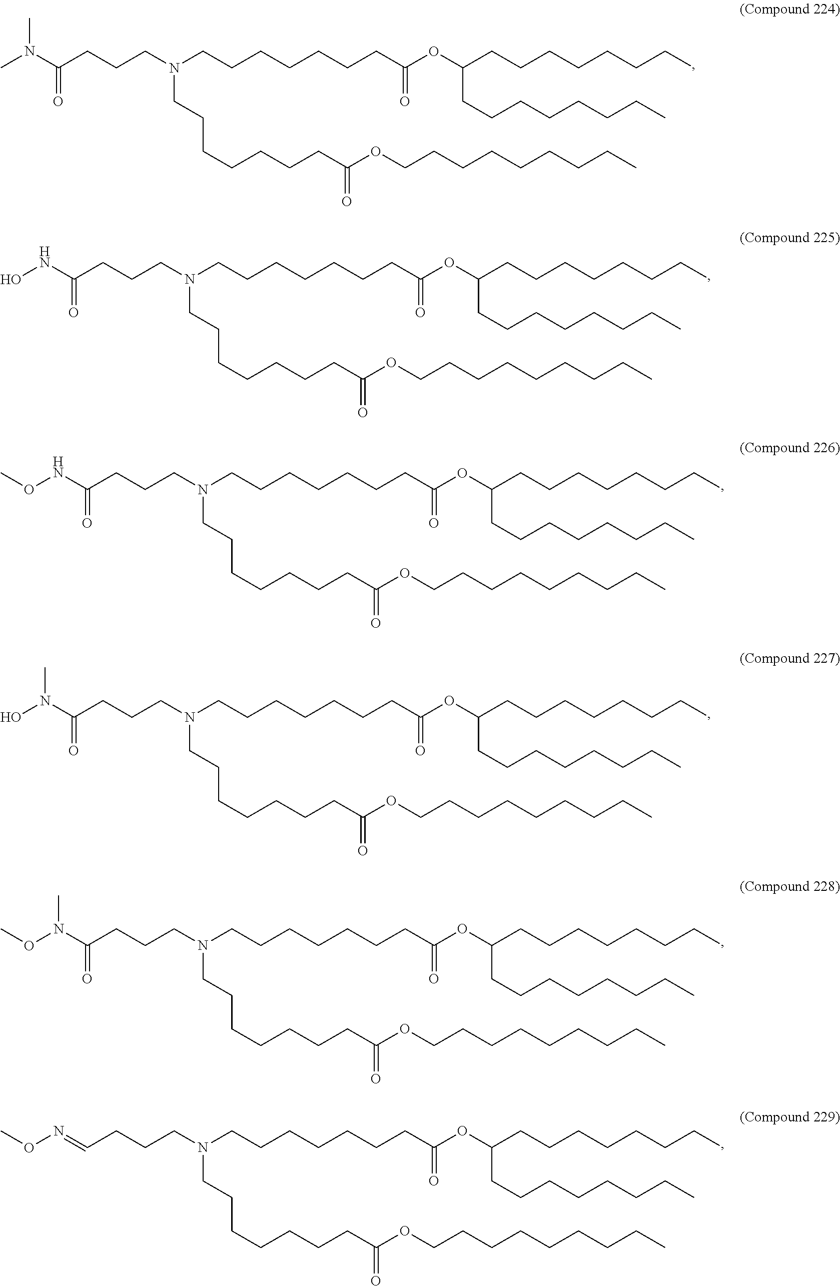

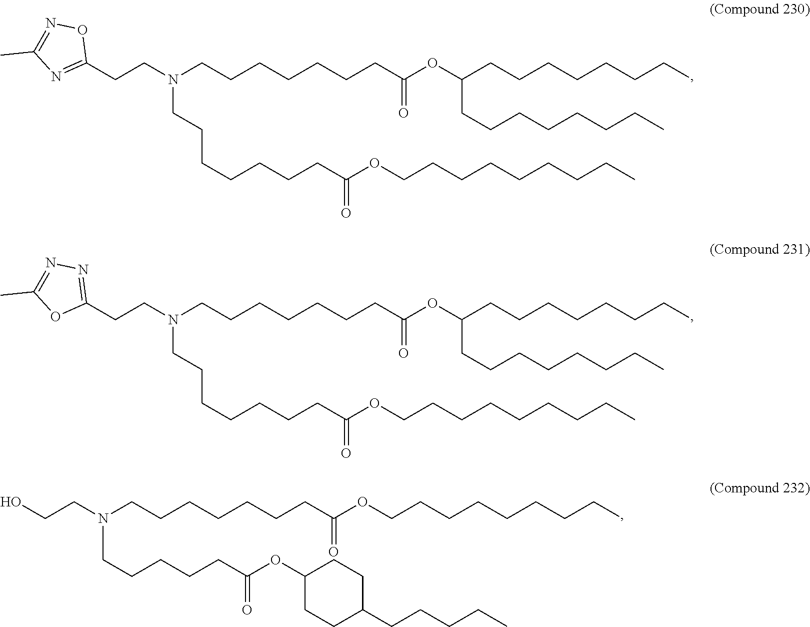

and salts and isomers thereof.

In some embodiments, a nano article comprises the following compound:

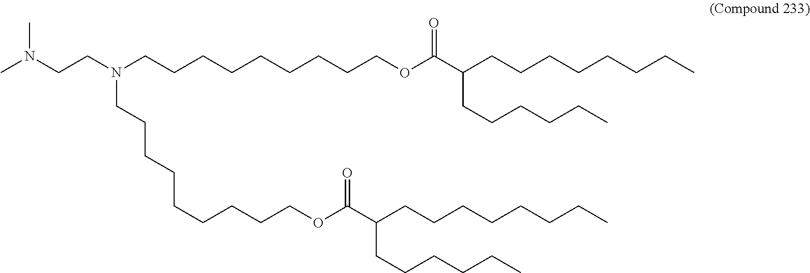 [SMILES: CCCCCCCCC(CCCCCC)C(=O)OCCCCCCCCCN(CCCCCCCCCOC(=O)C(CCCCCC)CCCCCCCC)CCN(C)C]

or salts and isomers thereof.

In some embodiments, the disclosure features a nanoparticle composition including a lipid component comprising a compound as described herein (e.g., a compound according to Formula (I), (IA), (II), (IIa), (IIb), (IIc), (IId) or (IIe)).

In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)-N,N-dimethyl-2-nonylhenicosa-12,15-dien-1-amine (L608), and N,N-dimethyl-1-[(1S,2R)-2-octylcyclopropyl]heptadecan-8-amine (L530).

In some embodiments, the lipid is

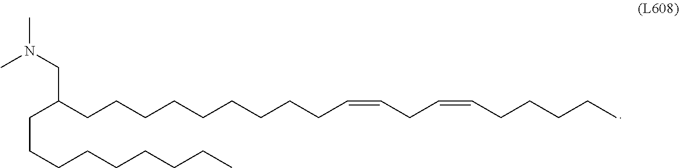 [SMILES: CCCCC/C=C\C/C=C\CCCCCCCCCC(CCCCCCCCC)CN(C)C]

In some embodiments, the lipid is

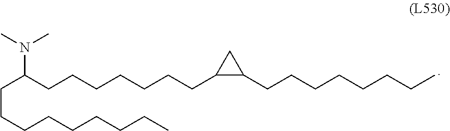 [SMILES: CCCCCCCCCC(CCCCCCCC1CC1CCCCCCCC)N(C)C]

### Multimeric Complexes

The RNA vaccines described herein can be assembled as multimeric complexes having non-covalent (e.g., hydrogen bonds) linkages between mRNA molecules. These types of multimeric structures allow for uniform distribution of the mRNA in a therapeutic composition. When multiple nucleic acids such as RNA are formulated, for instance, in a lipid based formulation, a relatively uniform distribution of the total nucleic acid through the formulation may be achieved. However, the distribution of a particular nucleic acid with respect to the other nucleic acids in the mixture is not uniform. For instance when the nucleic acid mixture is composed of two distinct mRNA sequences, some of the lipid particles or other formulatory agents will house a single mRNA sequence, while others will house the other mRNA sequence and a few will house both of the mRNA sequences. In a therapeutic context this uneven distribution of mRNA is undesirable because the dosage of the mRNA being delivered to a patient will vary from administration to administration. Quite surprisingly, the multimeric structures described herein have enabled the production of formulations having nucleic acids with a uniform distribution throughout the formulation. It was surprising that a non-covalent interaction between the individual nucleic acids would be capable of producing such a uniform distribution of the nucleic acids in a formulation. Additionally, the multimeric nucleic acid complexes do not interfere with activity such as mRNA expression activity.

In some embodiments the multimeric structures of the RNA polynucleotides making up the vaccine are uniformly distributed throughout a composition such as a lipid nanoparticle. Uniformly distributed, as used herein in the context of multiple nucleic acids (each having a unique nucleotide sequence), refers to the distribution of each of the nucleic acids relative to one another in the formulation. Distribution of the nucleic acids in a formulation may be assessed using methods known in the art. A nucleic acid is uniformly distributed relative to another nucleic acid if the nucleic acid is associated in proximity within a particular area of the formulation to the other nucleic acid at an approximately 1:1 ratio. In some embodiments the nucleic acid is uniformly distributed relative to another nucleic acid if the nucleic acid is positioned within a particular area of the formulation to the other nucleic acid at an approximately 1:1.1, 1:1.2, 1:1.3, 1:1.4, 1:1.5, 1:1.6, 1:1.7, 1:1.8, 1:1.9, or 1:2 ratio.

A multimeric structure as used herein is series of at least nucleic acids linked together to form a multimeric structure. In some embodiments a multimeric structure is composed of 2 or more, 3 or more, 4 or more, 5 or more 6 or more 7 or more, 8 or more, 9 or more nucleic acids. In other embodiments the multimeric structure is composed of 1000 or less, 900 or less, 500 or less, 100 or less, 75 or less, 50 or less, 40 or less, 30 or less, 20 or less or 100 or less nucleic acids. In yet other embodiments a multimeric structure has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 nucleic acids. In preferred embodiments a multimeric structure is composed of 3-5 nucleic acids.

In some embodiments the upper limit on the number of nucleic acids in a multimeric structure depends on the length of dimerizable region. A greater than 20-nucleotide space between mRNAs can provide specificity and enough force to keep the multi-mRNA complex intact for downstream processing and is thus preferred in some embodiments. In some embodiments 4-5 nucleic acids in a multimeric structure may be desirable for vaccines.

The multimeric structures may be self-assembling multimeric mRNA structures composed of a first mRNA having a first linking region comprised of a part A and a part B and a second mRNA having a second linking region comprised of a part C and a part D, wherein at least part A of the first and at least part C of the second linking regions are complementary to one another. Preferably the nucleic acids are linked to one another through a non-covalent bond in the linking regions. The following is an exemplary linking region, wherein X is any nucleic acid sequence of 0-100 nucleotides and A and B are complementary parts, which are complementary to one or more other nucleic acids.

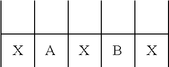

A linking region, as used herein, refers to a nucleic acid sequence having one or more regions or parts that are complementary to one or more regions of other linking regions. A pair of linking regions, each having one complementary region, may be at least 70% complementary to one another. In some embodiments a pair of linking regions are at least 75%, 80%, 85%, 90%, 95%, 96%, 97%, 98%, 99% or 100% complementary to one another. A linking region may be composed of sub-parts, optionally referred to as parts A, B, C, D, . . . , which have shorter regions of complementarity between one another, such that the subparts may be complementary with other sub-parts. For instance, a simple multimeric structure of two mRNAs can each have a linking region with a single region of complementarity. The two linking regions are able to form non-covalent interactions with one another through base pairing. More complex multimeric structures are also contemplated wherein a linking region of each nucleic acid has at least two parts, each part having complementarity with a part on another nucleic acid linking region. Linking regions having multiple parts with different complementarity enables the production of larger multimeric complexes of 3, 4, 5 or more nucleic acids.

The linking regions in some embodiments are 5-100 nucleotides in length. In other embodiments the linking regions are 10-25 nucleotides in length.

As used herein, the term “region of complementarity” refers to a region on a first nucleic acid strand that is substantially complementary to a second region on a second nucleic acid strand. Generally, two nucleic acids sharing a region of complementarity are capable, under suitable conditions, of hybridizing (e.g., via nucleic acid base pairing) to form a duplex structure. A region of complementarity can vary in size. In some embodiments, a region of complementarity ranges in length from about 2 base pairs to about 100 base pairs. In some embodiments, a region of complementarity ranges in length from about 5 base pairs to about 75 base pairs. In some embodiments, a region of complementarity ranges in length from about 10 base pairs to about 50 base pairs. In some embodiments, a region of complementarity ranges in length from about 20 base pairs to about 30 base pairs.

The number of nucleic acid bases shared between two nucleic acids across a region of complementarity can vary. In some embodiments, two nucleic acids share 100% complementary base pairs (e.g., no mismatches) across a region of complementarity. In some embodiments, two nucleic acids share at least 99.9%, at least 95%, at least 90%, at least 85%, at least 80%, at least 75% or at least 70% complementary base pairs across a region of complementarity. In some embodiments, a region of complementarity shared between two nucleic acids includes at least 1, at least 2, at least 3, at least 4, at least 5, at least 6, at least 7, at least 8, at least 9, or at least 10 base pair mismatches. In some embodiments, a region of complementarity shared between two nucleic acids includes more than 10 base pair mismatches.

As used herein, the term “non-covalent bond” refers to a chemical interaction (e.g., joining) between molecules that does not involve the sharing of electrons. Generally, non-covalent bonds are formed via electromagnetic interactions between charged molecules. Examples of non-covalent bonds include, but are not limited to, ionic bonds, hydrogen bonds, halogen bonds, Van der Waals forces (e.g., dipole-dipole interactions, London dispersion forces, etc.), π-effects (π-π interactions, cation-π interactions, anion-π interactions), and hydrophobic effect.

In some embodiments, at least one non-covalent bond formed between the nucleic acid molecules (e.g., mRNA molecules) of a multimeric molecule is a result of Watson-Crick base-pairing. The term “Watson-Crick base-pairing”, or “base-pairing” refers to the formation of hydrogen bonds between specific pairs of nucleotide bases (“complementary base pairs”). For example, two hydrogen bonds form between adenine (A) and uracil (U), and three hydrogen bonds form between guanine (G) and cytosine (C). One method of assessing the strength of bonding between two polynucleotides is by quantifying the percentage of bonds formed between the guanine and cytosine bases of the two polynucleotides (“GC content”). In some embodiments, the GC content of bonding between two nucleic acids of a multimeric molecule (e.g., a multimeric mRNA molecule) is at least 10%, at least 20%, at least 30%, at least 40%, or at least 50%. In some embodiments, the GC content of bonding between two nucleic acids of a multimeric molecule (e.g., a multimeric mRNA molecule) is between 10% and 70%, about 20% to about 60%, or about 30% to about 60%. The formation of a nucleic acid duplex via bonding of complementary base pairs can also be referred to as “hybridization”.

In some embodiments, two nucleic acid molecules (e.g., mRNA molecules) hybridize to form a multimeric molecule. Hybridization can result from the formation of at least 1, at least 2, at least 3, at least 4, at least 5, at least 6, at least 7, at least 8, at least 9, or at least 10 non-covalent bonds between two polynucleotides (e.g., mRNA molecules). In some embodiments, between about 2 non-covalent bonds and about 10 non-covalent bonds are formed between two nucleic acid molecules. In some embodiments, between about 5 and about 15 non-covalent bonds are formed between two nucleic acid molecules. In some embodiments, between about 10 and about 20 non-covalent bonds are formed between two nucleic acid molecules. In some embodiments, between about 15 and about 30 non-covalent bonds are formed between two nucleic acid molecules. In some embodiments, between about 20 and about 50 non-covalent bonds are formed between two nucleic acid molecules. In some embodiments, the number of non-covalent bonds formed between two nucleic acid molecules (e.g., mRNA molecules) is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 non-covalent bonds.

In some embodiments the self-assembling multimeric mRNA structure is comprised of at least 2-100 mRNAs each mRNA having a linking region and a stabilizing nucleic acid, wherein the stabilizing nucleic acid has a nucleotide sequence with regions complementary to each linking region. A stabilizing nucleic acid as used herein is any nucleic acid that has multiple linking regions and is capable of forming non-covalent interactions with at least 2, but more preferably, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 other nucleic acids. For instance the stabilizing nucleic acid may have the following structure:

L₁X₁L₂X₂L₃X₃L₄X₄L₅X₅L₆X₆ wherein L is a nucleic acid sequence complementary to a linking region and wherein x is any nucleic acid sequence 0-50 nucleotides in length. Such a structure may look like the following:

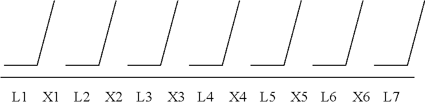

In some embodiments, a multimeric mRNA molecule comprises a first mRNA and a second mRNA, wherein the first mRNA and the second mRNA are non-covalently linked to one another through a splint. As used herein, the term “splint” refers to an oligonucleotide having a first region of complementarity with the first nucleic acid and a second region of complementarity with the second nucleic acid. A splint can be a DNA oligonucleotide or an RNA oligonucleotide. In some embodiments, a splint comprises one or more modified oligonucleotides. In some embodiments, a splint is non-covalently linked to a 5′UTR of an mRNA. In some embodiments, a splint is non-covalently linked to a 3′UTR of an mRNA.

In some embodiments, non-covalent bonds between nucleic acid molecules (e.g., mRNA molecules) are formed in a non-coding region of each molecule. As used herein, the term “non-coding region” refers to a location of a polynucleotide (e.g., an mRNA) that is not translated into a protein. Examples of non-coding regions include regulatory regions (e.g., DNA binding domains, promoter sequences, enhancer sequences), and untranslated regions (e.g., 5′UTR, 3′UTR). In some embodiments, the non-coding region is an untranslated region (UTR).

By definition, wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal.

Natural 5′UTRs bear features which play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

It should be understood that any UTR from any gene may be incorporated into the regions of the polynucleotide. Furthermore, multiple wild-type UTRs of any known gene may be utilized. It is also within the scope of the present invention to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ or 3′ UTR may be used. As used herein, a “double” UTR is one in which two copies of the same UTR are encoded either in series or substantially in series.

It is also within the scope of the present invention to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B. or C represent a different UTR at the nucleotide level.

In some embodiments, flanking regions are selected from a family of transcripts whose proteins share a common function, structure, feature of property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern. The untranslated region may also include translation enhancer elements (TEE).

In some embodiments, an UTR of a polynucleotide (e.g., a first nucleic acid) of the present invention is engineered or modified to have regions of complementarity with an UTR of another polynucleotide (a second nucleic acid). For example, UTR nucleotide sequences of two polynucleotides sought to be joined (e.g., in a multimeric molecule) can be modified to include a region of complementarity such that the two UTRs hybridize to form a multimeric molecule.

In some embodiments, the 5′UTR of an RNA polynucleotide encoding an HCMV antigenic polypeptide is modified to allow the formation of a multimeric sequence. In some embodiments, the 5′UTR of an RNA polynucleotide encoding an HCMV protein selected from gH, gL, gB, gO, gM, gM, UL128, UL130, UL131A1 is modified to allow the formation of a multimeric sequence. In some embodiments, the 5′UTR of an RNA polynucleotide encoding an HCMV protein selected from UL128, UL130. UL131A1 is modified to allow the formation of a multimeric sequence. In some embodiments, the 5′UTR of an RNA polynucleotide encoding an HCMV glycoprotein is modified to allow the formation of a multimeric sequence. In some embodiments, the 5′UTR of an RNA polynucleotide encoding an HCMV glycoprotein selected from gH, gL, gB, gO, gM, and gM is modified to allow the formation of a multimeric sequence. In any of these embodiments, the multimer may be a dimer, a trimer, pentamer, hexamer, heptamer, octamer nonamer, or decamer. In any of these embodiments, the multimer may be a homogenous multimer, that is, it may comprise dimers, trimers, pentamers etc having sequence encoding the same HCMV antigenic polypeptide. In any of these embodiments, the multimer may be a heterogeneous multimer comprising dimers, trimers, pentamers etc having sequence encoding different HCMV antigenic polypeptides, for example two different antigenic polypeptides, three different antigenic polypeptides, four different antigenic polypeptide, five different antigenic polypeptides, etc. Exemplary HCMV nucleic acids having modified 5′ UTR sequence for the formation of a multimeric molecule (e.g., dimers, trimers, pentamers, etc) comprise SEQ ID Nos: 19-26.

In some embodiments the RNA vaccine may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP²² derived or analog peptides, Pestivirus Erns, HSV, VP²² (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIs1, FGF, Lactoferrin, Transportan, Buforin-2, Bac715-24, SynB, SynB(1), pVEC, hCT-derived peptides, SAP, histones, cationic polysaccharides, for example chitosan, polybrene, cationic polymers, e.g. polyethyleneimine (PET), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O— ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammo-nium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In other embodiments the RNA vaccine is not associated with a cationic or polycationic compounds.

### Modes of Vaccine Administration

HCMV RNA vaccines may be administered by any route which results in a therapeutically effective outcome. These include, but are not limited, to intradermal, intramuscular, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. HCMV RNA vaccines compositions are typically formulated in dosage unit form for ease of administration and uniformity of dosage. It will be understood, however, that the total daily usage of HCMV RNA vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, HCMV RNA vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, HCMV RNA vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, HCMV RNA vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, HCMV RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later. Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a HCMV RNA vaccine composition may be administered three or four times.

In some embodiments, HCMV RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments the RNA vaccine for use in a method of vaccinating a subject is administered to the subject in a single dosage of between 10 μg/kg and 400 μg/kg of the nucleic acid vaccine in an effective amount to vaccinate the subject. In some embodiments the RNA vaccine for use in a method of vaccinating a subject is administered to the subject in a single dosage of between 10 μg and 400 μg of the nucleic acid vaccine in an effective amount to vaccinate the subject. In some embodiments, an HCMV RNA (e.g., mRNA) vaccine for use in a method of vaccinating a subject is administered to the subject in a single dosage of 10 μg. In some embodiments, an HCMV RNA vaccine for use in a method of vaccinating a subject is administered to the subject in a single dosage of 2 μg. In some embodiments, an HCMV RNA vaccine for use in a method of vaccinating a subject is administered to the subject in two dosages of 10 μg. In some embodiments, an HCMV RNA vaccine for use in a method of vaccinating a subject is administered the subject two dosages of 2 μg.

HCMV vaccines described herein can contain multiple RNA polynucleotides. The RNA polynucleotides can be present in equal or different amounts within the vaccine. For example, a vaccine can comprise: an RNA polynucleotide having an open reading frame encoding HCMV antigenic polypeptide gH, or an antigenic fragment or epitope thereof; an RNA polynucleotide having an open reading frame encoding HCMV antigenic polypeptide gL, or an antigenic fragment or epitope thereof; an RNA polynucleotide having an open reading frame encoding HCMV antigenic polypeptide UL128, or an antigenic fragment or epitope thereof; an RNA polynucleotide having an open reading frame encoding HCMV antigenic polypeptide UL130, or an antigenic fragment or epitope thereof; an RNA polynucleotide having an open reading frame encoding HCMV antigenic polypeptide UL131A, or an antigenic fragment or epitope thereof; and/or an RNA polynucleotide having an open reading frame encoding HCMV antigenic polypeptide gB, or an antigenic fragment or epitope thereof. In some embodiments, the ratio of gH-gL-UL128-UL130-UL131A is approximately 1:1:1:1:1. In other embodiments, the ratio of gH-gL-UL128-UL130-UL131A is approximately 4:2:1:1:1. In some embodiments, the ratio of gB-gH-gL-UL128-UL130-UL131A is approximately 1:1:1:1:1:1. In some embodiments, the vaccine comprises an equimolar concentration of gH, gL, UL128, UL130, and UL131A. In some embodiments, the vaccine comprises an equimolar concentration of gB, gH, gL, UL128, UL130, and UL131A. In some embodiments, the vaccine comprises an equal mass of gH, gL, UL128, UL130, and UL131A. In some embodiments, the vaccine comprises an equal mass of gB, gH, gL, UL128, UL130, and UL131A.

An HCMV RNA vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### HCMV RNA Vaccine Formulations and Methods of Use

Some aspects of the present disclosure provide formulations of one or more HCMV RNA (e.g., mRNA) vaccines, wherein the HCMV RNA vaccines are formulated in an effective amount to produce an antigen specific immune response in a subject (e.g., production of antibodies specific to an anti-HCMV antigenic polypeptide). “An effective amount” is a dose of an HCMV RNA (e.g., mRNA) vaccine effective to produce an antigen-specific immune response. Also provided herein are methods of inducing an antigen-specific immune response in a subject.

In some embodiments, the antigen-specific immune response is characterized by measuring an anti-HCMV antigenic polypeptide antibody titer produced in a subject administered an HCMV RNA (e.g., mRNA) vaccine as provided herein. An antibody titer is a measurement of the amount of antibodies within a subject, for example, antibodies that are specific to a particular antigen (e.g., an anti-HCMV antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the HCMV RNA vaccine.

In some embodiments, an anti-HCMV antigenic polypeptide antibody titer produced in a subject is increased by at least 1 log relative to a control. For example, anti-HCMV antigenic polypeptide antibody titer produced in a subject may be increased by at least 1.5, at least 2, at least 2.5, or at least 3 log relative to a control. In some embodiments, the anti-HCMV antigenic polypeptide antibody titer produced in the subject is increased by 1, 1.5, 2, 2.5 or 3 log relative to a control. In some embodiments, the anti-HCMV antigenic polypeptide antibody titer produced in the subject is increased by 1-3 log relative to a control. For example, the anti-HCMV antigenic polypeptide antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-HCMV antigenic polypeptide antibody titer produced in a subject is increased at least 2 times relative to a control. For example, the anti-HCMV antigenic polypeptide antibody titer produced in a subject may be increased at least 3 times, at least 4 times, at least 5 times, at least 6 times, at least 7 times, at least 8 times, at least 9 times, or at least 10 times relative to a control. In some embodiments, the anti-HCMV antigenic polypeptide antibody titer produced in the subject is increased 2, 3, 4, 5, 6, 7, 8, 9, or 10 times relative to a control. In some embodiments, the anti-HCMV antigenic polypeptide antibody titer produced in a subject is increased 2-10 times relative to a control. For example, the anti-HCMV antigenic polypeptide antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-HCMV antigenic polypeptide antibody titer produced in a subject who has not been administered an HCMV RNA (e.g., mRNA) vaccine. In some embodiments, a control is an anti-HCMV antigenic polypeptide antibody titer produced in a subject who has been administered a live attenuated HCMV vaccine. An attenuated vaccine is a vaccine produced by reducing the virulence of a viable (live). An attenuated virus is altered in a manner that renders it harmless or less virulent relative to live, unmodified virus. In some embodiments, a control is an anti-HCMV antigenic polypeptide antibody titer produced in a subject administered inactivated HCMV vaccine. In some embodiments, a control is an anti-HCMV antigenic polypeptide antibody titer produced in a subject administered a recombinant or purified HCMV protein vaccine. Recombinant protein vaccines typically include protein antigens that either have been produced in a heterologous expression system (e.g., bacteria or yeast) or purified from large amounts of the pathogenic organism.

In some embodiments, an effective amount of an HCMV RNA (e.g., mRNA) vaccine is a dose that is reduced compared to the standard of care dose of a recombinant HCMV protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified HCMV protein vaccine, or a live attenuated or inactivated HCMV vaccine, that a physician/clinician or other medical professional would administer to a subject to treat or prevent HCMV, or an HCMV-related condition, while following the standard of care guideline for treating or preventing HCMV, or an HCMV-related condition.

In some embodiments, the anti-HCMV antigenic polypeptide antibody titer produced in a subject administered an effective amount of an HCMV RNA vaccine is equivalent to an anti-HCMV antigenic polypeptide antibody titer produced in a control subject administered a standard of care dose of a recombinant or purified HCMV protein vaccine or a live attenuated or inactivated HCMV vaccine.

In some embodiments, an effective amount of an HCMV RNA (e.g., mRNA) vaccine is a dose equivalent to an at least 2-fold reduction in a standard of care dose of a recombinant or purified HCMV protein vaccine. For example, an effective amount of an HCMV RNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified HCMV protein vaccine. In some embodiments, an effective amount of an HCMV RNA vaccine is a dose equivalent to an at least at least 100-fold, at least 500-fold, or at least 1000-fold reduction in a standard of care dose of a recombinant or purified HCMV protein vaccine. In some embodiments, an effective amount of an HCMV RNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified HCMV protein vaccine. In some embodiments, the anti-HCMV antigenic polypeptide antibody titer produced in a subject administered an effective amount of an HCMV RNA vaccine is equivalent to an anti-HCMV antigenic polypeptide antibody titer produced in a control subject administered the standard of care dose of a recombinant or protein HCMV protein vaccine or a live attenuated or inactivated HCMV vaccine. In some embodiments, an effective amount of an HCMV RNA (e.g., mRNA) vaccine is a dose equivalent to a 2-fold to 1000-fold (e.g., 2-fold to 100-fold, 10-fold to 1000-fold) reduction in the standard of care dose of a recombinant or purified HCMV protein vaccine, wherein the anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount of an HCMV RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-, 2 to 5-, 2 to 4-, 2 to 3-, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10- , 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 4 to 00-, 4 to 200-, 4 to 100-, 4 to 90-, 4 to 80-, 4 to 70-, 4 to 60-, 4 to 50-, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9-, 5 to 8-, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-, 7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-, 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-, 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-, 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant HCMV protein vaccine. In some embodiments, such as the foregoing, the anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine. In some embodiments, the effective amount is a dose equivalent to (or equivalent to an at least) 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 4360-, 470-, 480-, 490-, 500-, 510-, 520-, 530-, 540-, 550-, 560-, 5760-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820-, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant HCMV protein vaccine. In some embodiments, such as the foregoing, an anti-HCMV antigenic polypeptide antibody titer produced in the subject is equivalent to an anti-HCMV antigenic polypeptide antibody titer produced in a control subject administered the standard of care dose of a recombinant or purified HCMV protein vaccine or a live attenuated or inactivated HCMV vaccine.

In some embodiments, the effective amount of an HCMV RNA (e.g., mRNA) vaccine is a total dose of 50-1000 μg. In some embodiments, the effective amount of an HCMV RNA (e.g., mRNA) vaccine is a total dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 μg. In some embodiments, the effective amount of an HCMV RNA (e.g., mRNA) vaccine is a total dose of 50, 100, 150, 200, 250, 300, 350, 400, 450, 500, 550, 600, 650, 700, 750, 800, 850, 900, 950 or 1000 μg. In some embodiments, the effective amount is a dose of 25-500 μg administered to the subject a total of two times. In some embodiments, the effective amount of an HCMV RNA (e.g., mRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 μg administered to the subject a total of two times. In some embodiments, the effective amount of an HCMV RNA (e.g., mRNA) vaccine is a total dose of 25, 50, 100, 150, 200, 250, 300, 350, 400, 450, or 500 μg administered to the subject a total of two times.

In some embodiments, the antigen specific immune response induced by the HCMV RNA vaccines in a subject is the production of antibodies specific to an anti-HCMV antigenic polypeptide. In some embodiments, such antibodies are capable of neutralizing HCMV in an infected host. In some embodiments,, the antigen specific immune response induced by the HCMV RNA vaccines in a subject is antigen-specific T-cell response. Such T-cell response may provide immunity to the immunized animal (e.g., mice or human) against fution HCMV infenctions.

### Kits

The present disclosure also provides any of the above-mentioned compositions in kits. Aspects of the disclosure relate to kits comprising one or more HCMV vaccines. In some aspects, a kit comprises: (i) an HCMV vaccine comprising an RNA polynucleotide having an open reading frame encoding HCMV antigenic polypeptide pp65, or an antigenic fragment or epitope thereof, and/or (ii) an HCMV vaccine comprising at least one RNA polynucleotide having one or more open reading frames encoding HCMV antigenic polypeptides gH, gL, UL128, UL130, and/or UL131A, or antigenic fragments or epitopes thereof; an RNA polynucleotide having an open reading frame encoding HCMV antigenic polypeptide gB, or an antigenic fragment or epitope thereof; and an RNA polynucleotide having an open reading frame encoding HCMV antigenic polypeptide pp65, or an antigenic fragment or epitope thereof.

In certain embodiments, instructions are provided for administering the one or more HCMV vaccines. The kit can include a description of use of the composition(s) for participation in any biological or chemical mechanism disclosed herein. The kits can further include a description of activity of the condition in treating the pathology, as opposed to the symptoms of the condition. That is, the kit can include a description of use of the compositions as discussed herein. The kit also can include instructions for use of a combination of two or more compositions of the invention, or instruction for use of a combination of a composition of the invention and other products. Instructions also may be provided for administering the composition by any suitable technique as previously described.

The kits described herein may also contain one or more containers, which may contain the composition and other ingredients as previously described. The kits also may contain instructions for mixing, diluting, and/or administering or applying the compositions of the invention in some cases. The compositions of the kit may be provided as any suitable form.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1: Manufacture of Polynucleotides

According to the present disclosure, the manufacture of polynucleotides and or parts or regions thereof may be accomplished utilizing the methods taught in International Application WO2014/152027 entitled “Manufacturing Methods for Production of RNA Transcripts”, the contents of which is incorporated herein by reference in its entirety.

Purification methods may include those taught in International Application WO2014/152030 and WO2014/152031, each of which is incorporated herein by reference in its entirety.

Detection and characterization methods of the polynucleotides may be performed as taught in WO2014/144039, which is incorporated herein by reference in its entirety.

Characterization of the polynucleotides of the disclosure may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, WO2014/144711 and WO2014/144767, the contents of each of which is incorporated herein by reference in its entirety.

### Example 2: Chimeric Polynucleotide Synthesis

### Introduction

According to the present disclosure, two regions or parts of a chimeric polynucleotide may be joined or ligated using triphosphate chemistry.

According to this method, a first region or part of 100 nucleotides or less is chemically synthesized with a 5′ monophosphate and terminal 3′desOH or blocked OH. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

It is noted that for ligation methods, ligation with DNA T4 ligase, followed by treatment with DNAse should readily avoid concatenation.

The entire chimeric polynucleotide need not be manufactured with a phosphate-sugar backbone. If one of the regions or parts encodes a polypeptide, then it is preferable that such region or part comprise a phosphate-sugar backbone.

Ligation is then performed using any known click chemistry, orthoclick chemistry, solulink, or other bioconjugate chemistries known to those in the art.

### Synthetic Route

The chimeric polynucleotide is made using a series of starting segments. Such segments include:
(a) Capped and protected 5′ segment comprising a normal 3′OH (SEG. 1)
(b) 5′ triphosphate segment which may include the coding region of a polypeptide and comprising a normal 3′OH (SEG. 2)
(c) 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) is treated with cordycepin and then with pyrophosphatase to create the 5′monophosphate.

Segment 2 (SEG. 2) is then ligated to SEG. 3 using RNA ligase. The ligated polynucleotide is then purified and treated with pyrophosphatase to cleave the diphosphate. The treated SEG.2-SEG. 3 construct is then purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF (SEG. 2) and 3′UTR+PolyA (SEG. 3).

The yields of each step may be as much as 90-95%.

### Example 3: PCR for cDNA Production

PCR procedures for the preparation of cDNA are performed using 2× KAPA HIFI™ HotStart ReadyMix by Kapa Biosystems (Woburn, MA). This system includes 2× KAPA ReadyMix 12.5 μl; Forward Primer (10 μM) 0.75 μl; Reverse Primer (10 μM) 0.75 μl; Template cDNA−100 ng; and dH₂O diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reaction is cleaned up using Invitrogen's PURELINK™ PCR Micro Kit (Carlsbad, CA) per manufacturer's instructions (up to 5 μg). Larger reactions will require a cleanup using a product with a larger capacity. Following the cleanup, the cDNA is quantified using the NANODROP® and analyzed by agarose gel electrophoresis to confirm the cDNA is the expected size. The cDNA is then submitted for sequencing analysis before proceeding to the in vitro transcription reaction.

### Example 4: In Vitro Transcription (IVT)

The in vitro transcription reaction generates polynucleotides containing uniformly modified polynucleotides. Such uniformly modified polynucleotides may comprise a region or part of the polynucleotides of the disclosure. The input nucleotide triphosphate (NTP) mix is made in-house using natural and un-natural NTPs.

A typical in vitro transcription reaction includes the following:

| | | | |
|---|---|---|---|
| 1 | Template cDNA | 1.0 | μg |
| 2 | 10x transcription buffer (400 mM | 2.0 | μl |
| | Tris-HCl pH 8.0, 190 mM MgCl₂, 50 mM | | |
| | DTT, 10 mM Spermidine) | | |
| 3 | Custom NTPs (25 mM each) | 7.2 | μl |
| 4 | RNase Inhibitor | 20 | U |
| 5 | T7 RNA polymerase | 3000 | U |

| | | |
|---|---|---|
| 6 | dH₂0 | Up to 20.0 μl. and |

| | |
|---|---|
| 7 | Incubation at 37° C. for 3 hr-5 hrs. |

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, TX) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

### Example 5: Enzymatic Capping

Capping of a polynucleotide is performed as follows where the mixture includes: IVT RNA 60 μg-180 μg and dH₂O up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U): Vaccinia capping enzyme (Guanylyl transferase) (40 U); dH₂O (Up to 28 μl); and incubation at 37° C. for 30 minutes for 60 μg RNA or up to 2 hours for 180 μg of RNA.

The polynucleotide is then purified using Ambion's MEGACLEAR™ Kit (Austin, TX) following the manufacturer's instructions. Following the cleanup, the RNA is quantified using the NANODROP™ (ThermoFisher, Waltham, MA) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 6: PolyA Tailing Reaction

Without a poly-T in the cDNA, a poly-A tailing reaction must be performed before cleaning the final product. This is done by mixing Capped IVT RNA (100 μl); RNase Inhibitor (20 U): 10× Tailing Buffer (0.5 M Tris-HCl (pH 8.0), 2.5 M NaCl, 100 mM MgCl₂)(12.0 μl); 20 mM ATP (6.0 μl); Poly-A Polymerase (20 U); dH₂O up to 123.5 μl and incubation at 37′ C for 30 min. If the poly-A tail is already in the transcript, then the tailing reaction may be skipped and proceed directly to cleanup with Ambion's MEGACLEAR™ kit (Austin, TX) (up to 500 μg). Poly-A Polymerase is preferably a recombinant enzyme expressed in yeast.

It should be understood that the processivity or integrity of the polyA tailing reaction may not always result in an exact size polyA tail. Hence polyA tails of approximately between 40-200 nucleotides, e.g, about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the invention.

### Example 7: Natural 5′ Caps and 5′ Cap Analogues

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-0 methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

When transfected into mammalian cells, the modified mRNAs have a stability of between 12-18 hours or more than 18 hours, e.g., 24, 36, 48, 60, 72 or greater than 72 hours.

### Example 8: Capping Assays

### A. Protein Expression Assay

Polynucleotides encoding a polypeptide, containing any of the caps taught herein can be transfected into cells at equal concentrations. 6, 12, 24 and 36 hours post-transfection the amount of protein secreted into the culture medium can be assayed by ELISA. Synthetic polynucleotides that secrete higher levels of protein into the medium would correspond to a synthetic polynucleotide with a higher translationally-competent Cap structure.

### B. Purity Analysis Synthesis

Polynucleotides encoding a polypeptide, containing any of the caps taught herein can be compared for purity using denaturing Agarose-Urea gel electrophoresis or HPLC analysis. Polynucleotides with a single, consolidated band by electrophoresis correspond to the higher purity product compared to polynucleotides with multiple bands or streaking bands. Synthetic polynucleotides with a single HPLC peak would also correspond to a higher purity product. The capping reaction with a higher efficiency would provide a more pure polynucleotide population.

### C. Cytokine Analysis

Polynucleotides encoding a polypeptide, containing any of the caps taught herein can be transfected into cells at multiple concentrations. 6, 12, 24 and 36 hours post-transfection the amount of pro-inflammatory cytokines such as TNF-alpha and IFN-beta secreted into the culture medium can be assayed by ELISA. Polynucleotides resulting in the secretion of higher levels of pro-inflammatory cytokines into the medium would correspond to a polynucleotides containing an immune-activating cap structure.

### D. Capping Reaction Efficiency

Polynucleotides encoding a polypeptide, containing any of the caps taught herein can be analyzed for capping reaction efficiency by LC-MS after nuclease treatment. Nuclease treatment of capped polynucleotides would yield a mixture of free nucleotides and the capped 5′-5-triphosphate cap structure detectable by LC-MS. The amount of capped product on the LC-MS spectra can be expressed as a percent of total polynucleotide from the reaction and would correspond to capping reaction efficiency. The cap structure with higher capping reaction efficiency would have a higher amount of capped product by LC-MS.

### Example 9: Agarose Gel Electrophoresis of Modified RNA or RT PCR Products

Individual polynucleotides (200-400 ng in a 20 μl volume) or reverse transcribed PCR products (200-400 ng) are loaded into a well on a non-denaturing 1.2% Agarose E-Gel (invitrogen, Carlsbad, CA) and run for 12-15 minutes according to the manufacturer protocol.

### Example 10: Nanodrop Modified RNA Quantification and UV Spectral Data

Modified polynucleotides in TE buffer (1 μl) are used for Nanodrop UV absorbance readings to quantitate the yield of each polynucleotide from an chemical synthesis or in vitro transcription reaction.

### Example 11: Formulation of Modified mRNA Using Lipidoids

Polynucleotides are formulated for in vitro experiments by mixing the polynucleotides with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations may used as a starting point. After formation of the particle, polynucleotide is added and allowed to integrate with the complex. The encapsulation efficiency is determined using a standard dye exclusion assays.

### Example 12: hCMV Vaccine—hCMV Glycoprotein Sequences

A hCMV vaccine may comprise, for example, at least one RNA polynucleotide encoded by at least one of the following sequences or by at least one fragment or epitope of the following sequences. In some embodiments, a hCMV vaccine may comprise at least one RNA polynucleotide comprising one of the mRNA sequences listed below or at least one fragment of one of the sequences listed below.

Throughout all of the Examples described herein, each of the sequences described herein can be a chemically modified sequence or an unmodified sequence which includes no nucleotide modifications.

Throughout all of the Examples described herein, open reading frame sequences can be linked to different 5′ and 3′UTRs.

Examples of UTR sequences include:

5′ UTR coding sequence:
(SEQ ID NO: 145)
TCAAGCTTTTGGACCCTCGTACAGAAGCTAATACGACTCACTATAGGGA
AATAAGAGAGAAAAGAAGAGTAAGAAGAAATATAAGAGCCACC
5′ UTR (without promoter) coding sequence:
(SEQ ID NO: 146)
GGGAAATAAGAGAGAAAAGAAGAGTAAGAAGAAATATAAGAGCCACC
3′ UTR coding sequence:
(SEQ ID NO: 147)
TGATAATAGGCTGGAGCCTCGGTGGCCATGCTTCTTGCCCCTTGGGCCT
CCCCCCAGCCCCTCCTCCCCTTCCTGCACCCGTACCCCCGTGGTCTTTG
AATAAAGTCTGAGTGGGCGGC.

5′UTR is bolded
3′UTR is underlined

hCMV-gH: hCMV, glycoprotein H (Merlin Strain)
(SEQ ID NO: 1)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCCAGGCCTCCCCTCCTACCTCATCAT
CCTCGCCGTCTGTCTCTTCAGCCACCTACTTTCGTCACGATATGGCGCAGAAGCCGTATCCG
AACCGCTGGACAAAGCGTTTCACCTACTGCTCAACACCTACGGGAGACCCATCCGCTTCCTG
CGTGAAAATACCACCCAGTGTACCTACAACAGCAGCCTCCGTAACAGCACGGTCGTCAGGGA
AAACGCCATCAGTTTCAACTTTTTCCAAAGCTATAATCAATACTATGTATTCCATATGCCTC
GATGTCTTTTTGCGGGTCCTCTGGCGGAGCAGTTTCTGAACCAGGTAGATCTGACCGAAACC
CTGGAAAGATACCAACAGAGACTTAACACTTACGCGCTGGTATCCAAAGACCTGGCCAGCTA
CCGATCTTTTTCGCAGCAGCTAAAGGCACAAGACAGCCTAGGTGAACAGCCCACCACTGTGC
CACCGCCCATTGACCTGTCAATACCTCACGTTTGGATGCCACCGCAAACCACTCCACACGGC
TGGACAGAATCACATACCACCTCAGGACTACACCGACCACACTTTAACCAGACCTGTATCCT
CTTTGATGGACACGATCTACTATTCAGCACCGTCACACCTTGTTTGCACCAAGGCTTTTACC
TCATCGACGAACTACGTTACGTTAAAATAACACTGACCGAGGACTTCTTCGTAGTTACGGTG
TCCATAGACGACGACACACCCATGCTGCTTATCTTCGGCCATCTTCCACGCGTACTTTTCAA
AGCGCCCTATCAACGCGACAACTTTATACTACGACAAACTGAAAAACACGAGCTCCTGGTGC
TAGTTAAGAAAGATCAACTGAACCGTCACTCTTATCTCAAAGACCCGGACTTTCTTGACGCC
GCACTTGACTTCAACTACCTAGACCTCAGCGCACTACTACGTAACAGCTTTCACCGTTACGC
CGTGGATGTACTCAAGAGCGGTCGATGTCAGATGCTGGACCGCCGCACGGTAGAAATGGCCT
TCGCCTACGCATTAGCACTGTTCGCAGCAGCCCGACAAGAAGAGGCCGGCGCCCAAGTCTCC
GTCCCACGGGCCCTAGACCGCCAGGCCGCACTCTTACAAATACAAGAATTTATGATCACCTG
CCTCTCACAAACACCACCACGCACCACGTTGCTGCTGTATCCCACGGCCGTGGACCTGGCCA
AACGAGCCCTTTGGACACCGAATCAGATCACCGACATCACCAGCCTCGTACGCCTGGTCTAC
ATACTCTCTAAACAGAATCAGCAACATCTCATCCCCCAATGGGCACTACGACAGATCGCCGA
CTTTGCCCTAAAACTACACAAAACGCACCTGGCCTCTTTTCTTTCAGCCTTCGCACGCCAAG
AACTClACCTCATGGGCAGCCTCGTCCACTCCATGCTGGTACATACGACGGAGAGACGCGAA
ATCTTCATCGTAGAAACGGGCCTCTGTTCATTGGCCGAGCTATCACACTTTACGCAGTTGTT
AGCTCATCCACACCACGAATACCTCAGCGACCTGTACACACCCTGTTCCAGTAGCGGGCGAC
GCGATCACTCGCTCGAACGCCTCACGCGTCTCTTCCCCGATGCCACCGTCCCCGCTACCGTT
CCCGCCGCCCTCTCCATCCTATCTACCATGCAACCAAGCACGCTGGAAACCTTCCCCGACCT
GTTTTGCTTGCCGCTCGGCGAATCCTTCTCCGCGCTGACCGTCTCCGAACACGTCAGTTATA
TCGTAACAAACCAGTACCTGATCAAAGGTATCTCCTACCCTGTCTCCACCACCGTCGTAGGC
CAGAGCCTCATCATCACCCAGACGGACAGTCAAACTAAATGCGAACTGACGCGCAACATGCA
TACCACACACAGCATCACAGTGGCGCTCAACATTTCGCTAGAAAACTGCGCCTTTTGCCAAA
GCGCCCTGCTAGAATACGACGACACGCAAGGCGTCATCAACATCATGTACATGCACGACTCG
GACGACGTCCTTTTCGCCCTGGATCCCTACAACGAAGTGGTGGTCTCATCTCCGCGAACTCA
CTACCTCATGCTTTTGAAAAACGGTACGGTACTAGAAGTAACTGACGTCGTCGTGGACGCCA
CCGACAGTCGTCTCcTCATGATGTCcGTCTACGCGCTATCGGCCATCATCGGCATCTATCTG
CTCTACCGCATGCTCAAGACATGCTGATAATAGGCTGGAGCCTCGGTGGCCATGCTTCTTGC
CCCTTGGGCCTCCCCCCAGCCCCTCCTCCCCTTCCTGCACCCGTACCCCCGTGGTCTTTGAA
TAAAGTCTGAGTGGGCGGC
hCMV-gH: hCMV, glycoprotein H (Merlin Strain) mRNA
(SEQ ID NO: 158)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCCAGGCCUCCCCUCCUACCUCAUCAU
CCUCGCCGUCUGUCUCUUCAGCCACCUACUUUCGUCACGAUAUGGCGCAGAAGCCGUAUCCG
AACCGCUGGACAAAGCGUUUCACCUACUGCUCAACACCUACGGGAGACCCAUCCGCUUCCUG
CGUGAAAAUACCACCCAGUGUACCUACAACAGCAGCCUCCGUAACAGCACGGUCGUCAGGGA
AAACGCCAUCAGUUUCAACUUUUUCCAAAGCUAUAAUCAAUACUAUGUAUUCCAUAUGCCUC
GAUGUCUUUUUGCGGGUCCUCUGGCGGAGCAGUUUCUGAACCAGGUAGAUCUGACCGAAACC
CUGGAAAGAUACCAACAGAGACUUAACACUUACGCGCUGGUAUCCAAAGACCUGGCCAGCUA
CCGAUCUUUUUCGCAGCAGCUAAAGGCACAAGACAGCCUAGGUGAACAGCCCACCACUGUGC
CACCGCCCAUUGACCUGUCAAUACCUCACGUUUGGAUGCCACCGCAAACCACUCCACACGGC
UGGACAGAAUCACAUACCACCUCAGGACUACACCGACCACACUUUAACCAGACCUGUAUCCU
CUUUGAUGGACACGAUCUACUAUUCAGCACCGUCACACCUUGUUUGCACCAAGGCUUUUACC
UCAUCGACGAACUACGUUACGUUAAAAUAACACUGACCGAGGACUUCUUCGUAGUUACGGUG
UCCAUAGACGACGACACACCCAUGCUGCUUAUCUUCGGCCAUCUUCCACGCGUACUUUUCAA
AGCGCCCUAUCAACGCGACAACUUUAUACUACGACAAACUGAAAAACACGAGCUCCUGGUGC
UAGUUAAGAAAGAUCAACUGAACCGUCACUCUUAUCUCAAAGACCCGGACUUUCUUGACGCC
GCACUUGACUUCAACUACCUAGACCUCAGCGCACUACUACGUAACAGCUUUCACCGUUACGC
CGUGGAUGUACUCAAGAGCGGUCGAUGUCAGAUGCUGGACCGCCGCACGGUAGAAAUGGCCU
UCGCCUACGCAUUAGCACUGUUCGCAGCAGCCCGACAAGAAGAGGCCGGCGCCCAAGUCUCC
GUCCCACGGGCCCUAGACCGCCAGGCCGCACUCUUACAAAUACAAGAAUUUAUGAUCACCUG
CCUCUCACAAACACCACCACGCACCACGUUGCUGCUGUAUCCCACGGCCGUGGACCUGGCCA
AACGAGCCCUUUGGACACCGAAUCAGAUCACCGACAUCACCAGCCUCGUACGCCUGGUCUAC
AUACUCUCUAAACAGAAUCAGCAACAUCUCAUCCCCCAAUGGGCACUACGACAGAUCGCCGA
CUUUGCCCUAAAACUACACAAAACGCACCUGGCCUCUUUUCUUUCAGCCUUCGCACGCCAAG
AACUCUACCUCAUGGGCAGCCUCGUCCACUCCAUGCUGGUACAUACGACGGAGAGACGCGAA
AUCUUCAUCGUAGAAACGGGCCUCUGUUCAUUGGCCGAGCUAUCACACUUUACGCAGUUGUU
AGCUCAUCCACACCACGAAUACCUCAGCGACCUGUACACACCCUGUUCCAGUAGCGGGCGAC
GCGAUCACUCGCUCGAACGCCUCACGCGUCUCUUCCCCGAUGCCACCGUCCCCGCUACCGUU
CCCGCCGCCCUCUCCAUCCUAUCUACCAUGCAACCAAGCACGCUGGAAACCUUCCCCGACCU
GUUUUGCUUGCCGCUUGGCGAAUCCUUCUCCGUGCUGACCGUCUCCGAACACGUGAGUUAUA
UCGUAACAAACCAGUACCUGAUCAAAGGUAUCUCCUACCCUGUCUCCACCACCGUCGUAGGC
CAGAGCCUCAUCAUCACCCAGACGGACAGUCAAACUAAAUGCGAACUGACGCGCAACAUGCA
UACCACACACAGCAUCACAGUGGCGCUCAACAUUUCGCUAGAAAACUGCGCCUUUUGCCAAA
GCGCCCUGCUAGAAUACGACGACACGCAAGGCGUCAUCAACAUCAUGUACAUGCACGACUCG
GACGACGUCCUUUUCGCCCUGGAUCCCUACAACGAAGUGGUGGUCUCAUCUCCGCGAACUCA
CUACCUCAUGCUUUUGAAAAACGGUACGGUACUAGAAGUAACUGACGUCGUCGUGGACGCCA
CCGACAGUCGUCUCCUCAUGAUGUCCGUCUACGCGCUAUCGGCCAUCAUCGGCAUCUAUCUG
CUCUACCGCAUGCUCAAGACAUGCUGAUAAUAGGCUGGAGCCUCGGUGGCCAUGCUUCUUGC
CCCUUGGGCCUCCCCCCAGCCCCUCCUCCCCUUCCUGCACCCGUACCCCCGUGGUCUUUGAA
UAAAGUCUGAGUGGGCGGC
hCMV-gHFLAG, hCMV glycoproteinH-FLAG tag
(SEQ ID NO: 2)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCCAGGCCTCCCCTCCTACCTCATCAT
CCTCGCCGTCTGTCTCTTCAGCCACCTACTTTCGTCACGATATGGCGCAGAAGCCGTATCCG
AACCGCTGGACAAAGCGTTTCACCTACTGCTCAACACCTACGGGAGACCCATCCGCTTCCTG
CGTGAAAATACCACCCAGTGTACCTACAACAGCAGCCTCCGTAACAGCACGGTCGTCAGGGA
AAACGCCATCAGTTTCAACTTTTTCCAAAGCTATAATCAATACTATGTATTCCATATGCCTC
GATGTCTTTTTGCGGGTCCTCTGGCGGAGCAGTTTCTGAACCAGGTAGATCTGACCGAAACC
CTGGAAAGATACCAACAGAGACTTAACACTTACGCGCTGGTATCCAAAGACCTGGCCAGCTA
CCGATCTTTTTCGCAGCAGCTAAAGGCACAAGACAGCCTAGGTGAACAGCCCACCACTGTGC
CACCGCCCATTGACCTGTCAATACCTCACGTTTGGATGCCACCGCAAACCACTCCACACGGC
TGGACAGAATCACATACCACCTCAGGACTACACCGACCACACTTTAACCAGACCTGTATCCT
CTTTGATGGACACGATCTACTATTCAGCACCGTCACACCTTGTTTGCACCAAGGCTTTTACC
TCATCGACGAACTACGTTACGTTAAAATAACACTGACCGAGGACTTCTTCGTAGTTACGGTG
TCCATAGACGACGACACACCCATGCTGCTTATCTTCGGCCATCTTCCACGCGTACTTTTCAA
AGCGCCCTATCAACGCGACAACTTTATACTACGACAAACTGAAAAACACGAGCTCCTGGTGC
TAGTTAAGAAAGATCAACTGAACCGTCACTCTTATCTCAAAGACCCGGACTTTCTTGACGCC
GCACTTGACTTCAACTACCTAGACCTCAGCGCACTACTACGTAACAGCTTTCACCGTTACGC
CGTGGATGTACTCAAGAGCGGTCGATGTCAGATGCTGGACCGCCGCACGGTAGAAATGGCCT
TCGCCTACGCATTAGCACTGTTCGCAGCAGCCCGACAAGAAGAGGCCGGCGCCCAAGTCTCC
GTCCCACGGGCCCTAGACCGCCAGGCCGCACTCTTACAAATACAAGAATTTATGATCACCTG
CCTCTCACAAACACCACCACGCACCACGTTGCTGCTGTATCCCACGGCCGTGGACCTGGCCA
AACGAGCCCTTTGGACACCGAATCAGATCACCGACATCACCAGCCTCGTACGCCTGGTCTAC
ATACTCTCTAAACAGAATCAGCAACATCTCATCCCCCAATGGGCACTACGACAGATCGCCGA
CTTTGCCCTAAAACTACACAAAACGCACCTGGCCTCTTTTCTTTCAGCCTTCGCACGCCAAG
AACTCTACCTCATGGGCAGCCTCGTCCACTCCATGCTGGTACATACGACGGAGAGACGCGAA
ATCTTCATCGTAGAAACGGGCCTCTGTTCATTGGCCGAGCTATCACACTTTACGCAGTTGTT
AGCTCATCCACACCACGAATACCTCAGCGACCTGTACACACCCTGTTCCAGTAGCGGGCGAC
GCGATCACTCGCTCGAACGCCTCACGCGTCTCTTCCCCGATGCCACCGTCCCCGCTACCGTT
CCCGCCGCCCTCTCCATCCTATCTACCATGCAACCAAGCACGCTGGAAACCTTCCCCGACCT
GTTTTGCTTGCCGCTCGGCGAATCCTTCTCCGCGCTGACCGTCTCCGAACACGTCAGTTATA
TCGTAACAAACCAGTACCTGATCAAAGGTATCTCCTACCCTGTCTCCACCACCGTCGTAGGC
CAGAGCCTCATCATCACCCAGACGGACAGTCAAACTAAATGCGAACTGACGCGCAACATGCA
TACCACACACAGCATCACAGTGGCGCTCAACATTTCGCTAGAAAACTGCGCCTTTTGCCAAA
GCGCCCTGCTAGAATACGACGACACGCAAGGCGTCATCAACATCATGTACATGCACGACTCG
GACGACGTCCTTTTCGCCCTGGATCCCTACAACGAAGTGGTGGTCTCATCTCCGCGAACTCA
CTACCTCATGCTTTTGAAAAACGGTACGGTACTAGAAGTAACTGACGTCGTCGTGGACGCCA
CCGACAGTCGTCTCCTCATGATGTCCGTCTACGCGCTATCGGCCATCATCGGCATCTATCTG
CTCTACCGCATGCTCAAGACATGCGATTACAAGGACGATGACGATAAGTGATGATAATAGGC
TGGAGCCTCGGTGGCCATGCTTCTTGCCCCTTGGGCCTCCCCCCAGCCCCTCCTCCCCTTCC
TGCACCCGTACCCCCGTGGTCTTTGAATAAAGTCTGAGTGGGCGGC
hCMV-gHFLAG, hCMV glycoproteinH-FLAG tag mRNA
(SEQ ID NO: 159)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCCAGGCCUCCCCUCCUACCUCAUCAU
CCUCGCCGUCUGUCUCUUCAGCCACCUACUUUCGUCACGAUAUGGCGCAGAAGCCGUAUCCG
AACCGCUGGACAAAGCGUUUCACCUACUGCUCAACACCUACGGGAGACCCAUCCGCUUCCUG
CGUGAAAAUACCACCCAGUGUACCUACAACAGCAGCCUCCGUAACAGCACGGUCGUCAGGGA
AAACGCCAUCAGUUUCAACUUUUUCCAAAGCUAUAAUCAAUACUAUGUAUUCCAUAUGCCUC
GAUGUCUUUUUGCGGGUCCUCUGGCGGAGCAGUUUCUGAACCAGGUAGAUCUGACCGAAACC
CUGGAAAGAUACCAACAGAGACUUAACACUUACGCGCUGGUAUCCAAAGACCUGGCCAGCUA
CCGAUCUUUUUCGCAGCAGCUAAAGGCACAAGACAGCCUAGGUGAACAGCCCACCACUGUGC
CACCGCCCAUUGACCUGUCAAUACCUCACGUUUGGAUGCCACCGCAAACCACUCCACACGGC
UGGACAGAAUCACAUACCACCUCAGGACUACACCGACCACACUUUAACCAGACCUGUAUCCU
CUUUGAUGGACACGAUCUACUAUUCAGCACCGUCACACCUUGUUUGCACCAAGGCUUUUACC
UCAUCGACGAACUACGUUACGUUAAAAUAACACUGACCGAGGACUUCUUCGUAGUUACGGUG
UCCAUAGACGACGACACACCCAUGCUGCUUAUCUUCGGCCAUCUUCCACGCGUACUUUUCAA
AGCGCCCUAUCAACGCGACAACUUUAUACUACGACAAACUGAAAAACACGAGCUCCUGGUGC
UAGUUAAGAAAGAUCAACUGAACCGUCACUCUUAUCUCAAAGACCCGGACUUUCUUGACGCC
GCACUUGACUUCAACUACCUAGACCUCAGCGCACUACUACGUAACAGCUUUCACCGUUACGC
CGUGGAUGUACUCAAGAGCGGUCGAUGUCAGAUGCUGGACCGCCGCACGGUAGAAAUGGCCU
UCGCCUACGCAUUAGCACUGUUCGCAGCAGCCCGACAAGAAGAGGCCGGCGCCCAAGUCUCC
GUCCCACGGGCCCUAGACCGCCAGGCCGCACUCUUACAAAUACAAGAAUUUAUGAUCACCUG
CCUCUCACAAACACCACCACGCACCACGUUGCUGCUGUAUCCCACGGCCGUGGACCUGGCCA
AACGAGCCCUUUGGACACCGAAUCAGAUCACCGACAUCACCAGCCUCGUACGCCUGGUCUAC
AUACUCUCUAAACAGAAUCAGCAACAUCUCAUCCCCCAAUGGGCACUACGACAGAUCGCCGA
CUUUGCCCUAAAACUACACAAAACGCACCUGGCCUCUUUUCUUUCAGCCUUCGCACGCCAAG
AACUCUACCUCAUGGGCAGCCUCGUCCACUCCAUGCUGGUACAUACGACGGAGAGACGCGAA
AUCUUCAUCGUAGAAACGGGCCUCUGUUCAUUGGCCGAGCUAUCACACUUUACGCAGUUGUU
AGCUCAUCCACACCACGAAUACCUCAGCGACCUGUACACACCCUGUUCCAGUAGCGGGCGAC
GCGAUCACUCGCUCGAACGCCUCACGCGUCUCUUCCCCGAUGCCACCGUCCCCGCUACCGUU
CCCGCCGCCCUCUCCAUCCUAUCUACCAUGCAACCAAGCACGCUGGAAACCUUCCCCGACCU
GUUUUGCUUGCCGCUCGGCGAAUCCUUCUCCGCGCUGACCGUCUCCGAACACGUCAGUUAUA
UCGUAACAAACCAGUACCUGAUCAAAGGUAUCUCCUACCCUGUCUCCACCACCGUCGUAGGC
CAGAGCCUCAUCAUCACCCAGACGGACAGUCAAACUAAAUGCGAACUGACGCGCAACAUGCA
UACCACACACAGCAUCACAGUGGCGCUCAACAUUUCGCUAGAAAACUGCGCCUUUUGCCAAA
GCGCCCUGCUAGAAUACGACGACACGCAAGGCGUCAUCAACAUCAUGUACAUGCACGACUCG
GACGACGUCCUUUUCGCCCUGGAUCCCUACAACGAAGUGGUGGUCUCAUCUCCGCGAACUCA
CUACCUCAUGCUUUUGAAAAACGGUACGGUACUAGAAGUAACUGACGUCGUCGUGGACGCCA
CCGACAGUCGUCUCCUCAUGAUGUCCGUCUACGCGCUAUCGGCCAUCAUCGGCAUCUAUCUG
CUCUACCGCAUGCUCAAGACAUGCGAUUACAAGGACGAUGACGAUAAGUGAUGAUAAUAGGC
UGGAGCCUCGGUGGCCAUGCUUCUUGCCCCUUGGGCCUCCCCCCAGCCCCUCCUCCCCUUCC
UGCACCCGUACCCCCGUGGUCUUUGAAUAAAGUCUGAGUGGGUGGC
hCMV-gL, hCMV glycoprotein L
(SEQ ID NO: 3)
TCAAGCTTTTGGACCCTCGTACAGAAGCTAATACGACTCACTATAGGGAAATAAGAGAGAAA
AGAAGAGTAAGAAGAAATATAAGAGCCACCATGTGCCGCCGCCCGGATTGCGGCTTCTCTTT
CTCACCTGGACCGGTGATACTGCTGTGGTGTTGCCTTCTGCTGCCCATTGTTTCCTCAGCCG
CCGTCAGCGTCGCTCCTACCGCCGCCGAGAAAGTCCCCGCGGAGTGCCCCGAACTAACGCGC
CGATGCTTGTTGGGTGAGGTGTTTGAGGGTGACAAGTATGAAAGTTGGCTGCGCCCGTTGGT
GAATGTTACCGGGCGCGATGGCCCGCTATCGCAACTTATCCGTTACCGTCCCGTTACGCCGG
AGGCCGCCAACTCCGTGCTGTTGGACGAGGCTTTCCTGGACACTCTGGCCCTGCTGTACAAC
AATCCGGATCAATTGCGGGCCCTGCTGACGCTGTTGAGCTCGGACACAGCGCCGCGCTGGAT
GACGGTGATGCGCGGCTACAGCGAGTGCGGCGATGGCTCGCCGGCCGTGTACACGTGCGTGG
ACGACCTGTGCCGCGGCTACGACCTCACGCGACTGTCATACGGGCGCAGCATCTTCACGGAA
CACGTGTTAGGCTTCGAGCTGGTGCCACCGTCTCTCTTTAACGTGGTGGTGGCCATACGCAA
CGAAGCCACGCGTACCAACCGCGCCGTGCGTCTGCCCGTGAGCACCGCTGCCGCGCCCGAGG
GCATCACGCTCTTTTACGGCCTGTACAACGCAGTGAAGGAATTCTGCCTGCGTCACCAGCTG
GACCCGCCGCTGCTACGCCACCTAGATAAATACTACGCCGGACTGCCGCCCGAGCTGAAGCA
GACGCGCGTCAACCTGCCGGCTCACTCGCGCTATGGCCCTCAAGCAGTGGATGCTCGCTGAT
AATAGGCTGGAGCCTCGGTGGCCATGCTTCTTGCCCCTTGGGCCTCCCCCCAGCCCCTCCTC
CCCTTCCTGCAC0CGTACCCCCGTGGTCTTTGAATAAAGTCTGAGTGGGCGGC
hCMV-gL, hCMV glycoprotein L mRNA
(SEQ ID NO: 160)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGUGCCGCCGCCCGGAUUGCGGCUUCUCUUU
CUCACCUGGACCGGUGAUACUGCUGUGGUGUUGCCUUCUGCUGCCCAUUGUUUCCUCAGCCG
CCGUCAGCGUCGCUCCUACCGCCGGCGAGAAAGUCCCCGCGGAGUGCCCCGAACUAACGCGC
CGAUGCUUGUUGGGUGAGGUGUUUGAGGGUGACAAGUAUGAAAGUUGGCUGCGCCCGUUGGU
GAAUGUUACCGGGCGCGAUGGCCCGCUAUCGCAACUUAUCCGUUACCGUCCCGUUACGCCGG
AGGCCGCCAACUCCGUGCUGUUGGACGAGGCUUUCCUGGACACUCUGGCCCUGCUGUACAAC
AAUCCGGAUCAAUUGCGGGCCCUGCUGACGCUGUUGAGCUCGGACACAGCGCCGCGCUGGAU
GACGGUGAUGCGCGGCUACAGCGAGUGCGGCGAUGGCUCGCCGGCCGUGUACACGUGCGUGG
ACGACCUGUGCCGCGGCUACGACCUCACGCGACUGUCAUACGGGCGCAGCAUCUUCACGGAA
CACGUGUUAGGCUUCGAGCUGGUGCCACCGUCUCUCUUUAACGUGGUGGUGGCCAUACGCAA
CGAAGCCACGCGUACCAACCGCGCCGUGCGUCUGCCCGUGAGCACCGCUGCCGCGCCCGAGG
GCAUCACGCUCUUUUACGGCCUGUACAACGCAGUGAAGGAAUUCUGCCUGCGUCACCAGCUG
GACCCGCCGCUGCUACGCCACCUAGAUAAAUACUACGCCGGACUGCCGCCCGAGCUGAAGCA
GACGCGCGUCAACCUGCCGGCUCACUCGCGCUAUGGCCCUCAAGCAGUGGAUGCUCGCUGAU
AAUAGGCUGGAGCCUCGGUGGCCAUGCUUCUUGCCCCUUGGGCCUCCCCCCAGCCCCUCCUC
CCCUUCCUGCACCCGUACCCCCGUGGUCUUUGAAUAAAGUCUGAGUGGGCGGC
hCMV-gLFLAG, glycoprotein L-FLAG
(SEQ ID NO: 4)
TCAAGCTTTTGGACCCTCGTACAGAAGCTAATACGACTCACTATAGGGAAATAAGAGAGAAA
AGAAGAGTAAGAAGAAATATAAGAGCCACCATGTGCCGCCGCCCGGATTGCGGCTTCTCTTT
CTCACCTGGACCGGTGATACTGCTGTGGTGTTGCCTTCTGCTGCCCATTGTTTCCTCAGCCG
CCGTCAGCGTCGCTCCTACCGCCGCCGAGAAAGTCCCCGCGGAGTGCCCCGAACTAACGCGC
CGATGCTTGTTGGGTGAGGTGTTTGAGGGTGACAAGTATGAAAGTTGGCTGCGCCCGTTGGT
GAATGTTACCGGGCGCGATGGCCCGCTATCGCAACTTATCCGTTACCGTCCCGTTACGCCGG
AGGCCGCCAACTCCGTGCTGTTGGACGAGGcTTTCCTGGACACTCTGGCCCTGCTGTACAAC
AATCCGGATCAATTGCGGGCCCTGCTGACGCTGTTGAGCTCGGACACAGCGCCGCGCTGGAT
GACGGTGATGCGCGGCTACAGCGAGTGCGGCGATGGCTCGCCGGCCGTGTACACGTGCGTGG
ACGACCTGTGCCGCGGCTACGACCTCACGCGACTGTCATACGGGCGCAGCATCTTCACGGAA
CACGTGTTAGGCTTCGAGCTGGTGCCACCGTCTCTCTTTAACGTGGTGGTGGCCATACGCAA
CGAAGCCACGCGTACCAACCGCGCCGTGCGTCTGCCCGTGAGCACCGCTGCCGCGCCCGAGG
GCATCACGCTCTTTTACGGCCTGTACAACGCAGTGAAGGAATTCTGCCTGCGTCACCAGCTG
GACCCGCCGCTGCTACGCCACCTAGATAAATACTACGCCGGACTGCCGCCCGAGCTGAAGCA
GACGCGCGTCAACCTGCCGGCTCACTCGCGCTATGGCCCTCAAGCAGTGGATGCTCGCGATT
ACAAGGACGATGACGATAAGTGATGATAATAGGCTGGAGCCTCGGTGGCCATGCTTCTTGCC
CCTTGGGCCTCCCCCCAGCCCCTCCTCCCCTTCCTGCACCCGTACCCCCGTGGTCTTTGAAT
AAAGTCTGAGTGGGCGGC
hCMV-gLFLAG, glycoprotein L-FLAG mRNA
(SEQ ID NO: 161)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGUGCCGCCGCCCGGAUUGCGGCUUCUCUUU
CUCACCUGGACCGGUGAUACUGCUGUGGUGUUGCCUUCUGCUGCCCAUUGUUUCCUCAGCCG
CCGUCAGCGUCGCUCCUACCGCCGCCGAGAAAGUCCCCGCGGAGUGCCCCGAACUAACGCGC
CGAUGCUUGUUGGGUGAGGUGUUUGAGGGUGACAAGUAUGAAAGUUGGCUGCGCCCGUUGGU
GAAUGUUACCGGGCGCGAUGGCCCGCUAUCGCAACUUAUCCGUUACCGUCCCGUUACGCCGG
AGGCCGCCAACUCCGUGCUGUUGGACGAGGCUUUCCUGGACACUCUGGCCCUGCUGUACAAC
AAUCCGGAUCAAUUGCGGGCCCUGCUGACGCUGUUGAGCUCGGACACAGCGCCGCGCUGGAU
GACGGUGAUGCGCGGCUACAGCGAGUGCGGCGAUGGCUCGCCGGCCGUGUACACGUGCGUGG
ACGACCUGUGCCGCGGCUACGACCUCACGCGACUGUCAUACGGGCGCAGCAUCUUCACGGAA
CACGUGUUAGGCUUCGAGCUGGUGCCACCGUCUCUCUUUAACGUGGUGGUGGCCAUACGCAA
CGAAGCCACGCGUACCAACCGCGCCGUGCGUCUGCCCGUGAGCACCGCUGCCGCGCCCGAGG
GCAUCACGCUCUUUUACGGCCUGUACAACGCAGUGAAGGAAUUCUGCCUGCGUCACCAGCUG
GACCCGCCGCUGCUACGCCACCUAGAUAAAUACUACGCCGGACUGCCGCCCGAGCUGAAGCA
GACGCGCGUCAACCUGCCGGCUCACUCGCGCUAUGGCCCUCAAGCAGUGGAUGCUCGCGAUU
ACAAGGACGAUGACGAUAAGUGAUGAUAAUAGGCUGGAGCCUCGGUGGCCAUGCUUCUUGCC
CCUUGGGCCUCCCCCCAGCCCCUCCUCCCCUUCCUGCACCCGUACCCCCGUGGUCUUUGAAU
AAAGUCUGAGUGGGCGGC
hCMV gB, hCMV glycoprotein B
(SEQ ID NO: 5)
TCAAGCTTTTGGACCCTCGTACAGAAGCTAATACGACTCACTATAGGGAAATAAGAGAGAAA
AGAAGAGTAAGAAGAAATATAAGAGCCACCATGGAATCCAGGATCTGGTGCCTGGTAGTCTG
CGTTAACTTGTGTATCGTCTGTCTGGGTGCTGCGGTTTCCTCATCTTCTACTCGTGGAACTT
CTGCTACTCACAGTCACCATTCCTCTCATACGACGTCTGCTGCTCACTCTCGATCCGGTTCA
GTCTCTCAACGCGTAACTTCTTCCCAAACGGTCAGCCATGGTGTTAACGAGACCATCTACAA
CACTACCCTCAAGTACGGAGATGTGGTGGGGGTCAATACCACCAAGTACCCCTATCGCGTGT
GTTCTATGGCCCAGGGTACGGATCTTATTCGCTTTGAACGTAATATCGTCTGCACCTCGATG
AAGCCCATCAATGAAGACCTGGACGAGGGCATCATGGTGGTCTACAAACGCAACATCGTCGC
GCACACCTTTAAGGTACGAGTCTACCAGAAGGTTTTGACGTTTCGTCGTAGCTACGCTTACA
TCCACACCACTTATCTGCTGGGCAGCAACACGGAATACGTGGCGCCTCCTATGTGGGAGATT
CATCATATCAACAGCCACAGTCAGTGCTACAGTTCCTACAGCCGCGTTATAGCAGGCACGGT
TTTCGTGGCTTATCATAGGGACAGCTATGAAAACAAAACCATGCAATTAATGCCCGACGATT
ATTCCAACACCCACAGTACCCGTTACGTGACGGTCAAGGATCAATGGCACAGCCGCGGCAGC
ACCTGGCTCTATCGTGAGACCTGTAATCTGAATTGTATGGTGACCATCACTACTGCGCGCTC
CAAATATCCTTATCATTTTTTCGCCACTTCCACGGGTGACGTGGTTGACATTTCTCCTTTCT
ACAACGGAACCAATCGCAATGCCAGCTACTTTGGAGAAAACGCCGACAAGTTTTTCATTTTT
CCGAACTACACTATCGTCTCCGACTTTGGAAGACCGAATTCTGCGTTAGAGACCCACAGGTT
GGTGGCTTTTCTTGAACGTGCGGACTCGGTGATCTCCTGGGATATACAGGACGAAAAGAATG
TCACTTGTCAACTCACTTTCTGGGAAGCCTCGGAACGCACCATTCGTTCCGAAGCCGAGGAC
TCGTATCACTTTTCTTCTGCCAAAATGACCGCCACTTTCTTATCTAAGAAGCAAGAGGTGAA
CATGTCCGACTCTGCGCTGGACTGCGTACGTGATGAGGCTATAAATAAGTTACAGCAGATTT
TCAATACTTCATACAATCAAACATATGAAAAATATGGAAACGTGTCCGTCTTTGAAACCACT
GGTGGTTTGGTAGTGTTCTGGCAAGGTATCAAGCAAAAATCTCTGGTGGAACTCGAACGTTT
GGCCAACCGCTCCAGTCTGAATCTTACTCATAATAGAACCAAAAGAAGTACAGATGGCAACA
ATGCAACTCATTTATCCAACATGGAATCGGTGCACAATCTGGTCTACGCCCAGCTGCAGTTC
ACCTATGACACGTTGCGCGGTTACATCAACCGGGCGCTGGCGCAAATCGCAGAAGCCTGGTG
TGTGGATCAACGGCGCACCCTAGAGGTCTTCAAGGAACTCAGCAAGATCAACCCGTCAGCCA
TTCTCTCGGCCATTTACAACAAACCGATTGCCGCGCGTTTCATGGGTGATGTCTTGGGCCTG
GCCAGCTGCGTGACCATCAACCAAACCAGCGTCAAGGTGCTGCGTGATATGAACGTGAAGGA
GTCGCCAGGACGCTGCTACTCACGACCCGTGGTCATCTTTAATTTCGCCAACAGCTCGTACG
TGCAGTACGGTCAACTGGGCGAGGACAACGAAATCCTGTTGGGCAACCACCGCACTGAGGAA
TGTCAGCTTCCCAGCCTCAAGATCTTCATCGCCGGGAACTCGGCCTACGAGTACGTGGACTA
CCTCTTCAAACGCATGATTGACCTCAGCAGTATCTCCACCGTCGACAGCATGATCGCCCTGG
ATATCGACCCGCTGGAAAATACCGACTTCAGGGTACTGGAACTTTACTCGCAGAAAGAGCTG
CGTTCCAGCAACGTTTTTGACCTCGAAGAGATCATGCGCGAATTCAACTCGTACAAGCAGCG
GGTAAAGTACGTGGAGGACAAGGTAGTCGACCCGCTACCGCCCTACCTCAAGGGTCTGGACG
ACCTCATGAGCGGCCTGGGCGCCGCGGGAAAGGCCGTTGGCGTAGCCATTGGGGCCGTGGGT
GGCGCGGTGGCCTCCGTGGTCGAAGGCGTTGCCACCTTCCTCAAAAACCCCTTCGGAGCGTT
CACCATCATCCTCGTGGCCATAGCTGTAGTCATTATCACTTATTTGATCTATACTCGACAGC
GGCGTTTGTGCACGCAGCCGCTGCAGAACCTCTTTCCCTATCTGGTGTCCGCCGACGGGACC
ACCGTGACGTCGGGCAGCACCAAAGACACGTCGTTACAGGCTCCGCCTTCCTACGAGGAAAG
TGTTTATAATTCTGGTCGCAAAGGACCGGGACCACCGTCGTCTGATGCATCCACGGCGGCTC
CGCCTTACACCAACGAGCAGGCTTACCAGATGCTTCTGGCCCTGGCCCGTCTGGACGCAGAG
CAGCGAGCGCAGCAGAACGGTACAGATTCTTTGGACGGACGGACTGGCACGCAGGACAAGGG
ACAGAAGCCCAACCTACTAGACCGACTGCGACATCGCAAAAACGGCTACCGACACTTGAAAG
ACTCTGACGAAGAAGAGAACGTCTGATAATAGGCTGGAGCCTCGGTGGCCATGCTTCTTGCC
CCTTGGGCCTCCCCCCAGCCCCTCCTCCCCTTCCTGCACCCGTACCCCCGTGGTCTTTGAAT
AAAGTCTGAGTGGGCGGC
hCMV_gB, hCMV glycoprotein B mRNA
(SEQ ID NO: 162)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGGAAUCCAGGAUCUGGUGCCUGGUAGUCUG
CGUUAACUUGUGUAUCGUCUGUCUGGGUGCUGCGGUUUCCUCAUCUUCUACUCGUGGAACUU
CUGCUACUCACAGUCACCAUUCCUCUCAUACGACGUCUGCUGCUCACUCUCGAUCCGGUUCA
GUCUCUCAACGCGUAACUUCUUCCCAAACGGUCAGCCAUGGUGUUAACGAGACCAUCUACAA
CACUACCCUCAAGUACGGAGAUGUGGUGGGGGUCAAUACCACCAAGUACCCCUAUCGCGUGU
GUUCUAUGGCCCAGGGUACGGAUCUUAUUCGCUUUGAACGUAAUAUCGUCUGCACCUCGAUG
AAGCCCAUCAAUGAAGACCUGGACGAGGGCAUCAUGGUGGUCUACAAACGCAACAUCGUCGC
GCACACCUUUAAGGUACGAGUCUACCAGAAGGUUUUGACGUUUCGUCGUAGCUACGCUUACA
UCCACACCACUUAUCUGCUGGGCAGCAACACGGAAUACGUGGCGCCUCCUAUGUGGGAGAUU
CAUCAUAUCAACAGCCACAGUCAGUGCUACAGUUCCUACAGCCGCGUUAUAGCAGGCACGGU
UUUCGUGGCUUAUCAUAGGGACAGCUAUGAAAACAAAACCAUGCAAUUAAUGCCCGACGAUU
AUUCCAACACCCACAGUACCCGUUACGUGACGGUCAAGGAUCAAUGGCACAGCCGCGGCAGC
ACCUGGCUCUAUCGUGAGACCUGUAAUCUGAAUUGUAUGGUGACCAUCACUACUGCGCGCUC
CAAAUAUCCUUAUCAUUUUUUCGCCACUUCCACGGGUGACGUGGUUGACAUUUCUCCUUUCU
ACAACGGAACCAAUCGCAAUGCCAGCUACUUUGGAGAAAACGCCGACAAGUUUUUCAUUUUU
CCGAACUACACUAUCGUCUCCGACUUUGGAAGACCGAAUUUUGCGUUAGAGACCCACAGGUU
GGUGGCUUUUCUUGAACGUGCGGACUCGGUGAUCUCCUGGGAUAUACAGGACGAAAAGAAUG
UCACUUGUCAACUCACUUUCUGGGAAGCCUCGGAACGCACCAUUCGUUCCGAAGCCGAGGAC
UCGUAUCACUUUUCUUCUGCCAAAAUGACCGCCACUUUCUUAUCUAAGAAGCAAGAGGUGAA
CAUGUCCGACUCUGCGCUGGACUGCGUACGUGAUGAGGCUAUAAAUAAGUUACAGCAGAUUU
UCAAUACUUCAUACAAUCAAACAUAUGAAAAAUAUGGAAACGUGUCCGUCUUUGAAACCACU
GGUGGUUUGGUAGUGUUCUGGCAAGGUAUCAAGCAAAAAUCUCUGGUGGAACUCGAACGUUU
GGCCAACCGCUCCAGUCUGAAUCUUACUCAUAAUAGAACCAAAAGAAGUACAGAUGGCAACA
AUGCAACUCAUUUAUCCAACAUGGAAUCGGUGCACAAUCUGGUCUACGCCCAGCUGCAGUUC
ACCUAUGACACGUUGCGCGGUUACAUCAACCGGGCGCUGGCGCAAAUCGCAGAAGCCUGGUG
UGUGGAUCAACGGCGCACCCUAGAGGUCUUCAAGGAACUCAGCAAGAUCAACCCGUCAGCCA
UUCUCUCGGCCAUUUACAACAAACCGAUUGCCGCGCGUUUCAUGGGUGAUGUCUUGGGCCUG
GCCAGCUGCGUGACCAUCAACCAAACCAGCGUCAAGGUGCUGCGUGAUAUGAACGUGAAGGA
GUCGCCAGGACGCUGCUACUCACGACCCGUGGUCAUCUUUAAUUUCGCCAACAGCUCGUACG
UGCAGUACGGUCAACUGGGCGAGGACAACGAAAUCCUGUUGGGCAACCACCGCACUGAGGAA
UGUCAGCUUCCCAGCCUCAAGAUCUUCAUCGCCGGGAACUCGGCCUACGAGUACGUGGACUA
CCUCUUCAAACGCAUGAUUGACCUCAGCAGUAUCUCCACCGUCGACAGCAUGAUCGCCCUGG
AUAUCGACCCGCUGGAAAAUACCGACUUCAGGGUACUGGAACUUUACUCGCAGAAAGAGCUG
CGUUCCAGCAACGUUUUUGACCUCGAAGAGAUCAUGCGCGAAUUCAACUCGUACAAGCAGCG
GGUAAAGUACGUGGAGGACAAGGUAGUCGACCCGCUACCGCCCUACCUCAAGGGUCUGGACG
ACCUCAUGAGCGGCCUGGGCGCCGCGGGAAAGGCCGUUGGCGUAGCCAUUGGGGCCGUGGGU
GGCGCGGUGGCCUCCGUGGUCGAAGGCGUUGCCACCUUCCUCAAAAACCCCUUCGGAGCGUU
CACCAUCAUCCUCGUGGCCAUAGCUGUAGUCAUUAUCACUUAUUUGAUCUAUACUCGACAGC
GGCGUUUGUGCACGCAGCCGCUGCAGAACCUCUUUCCCUAUCUGGUGUCCGCCGACGGGACC
ACCGUGACGUCGGGCAGCACCAAAGACACGUCGUUACAGGCUCCGCCUUCCUACGAGGAAAG
UGUUUAUAAUUCUGGUCGCAAAGGACCGGGACCACCGUCGUCUGAUGCAUCCACGGCGGCUC
CGCCUUACACCAACGAGCAGGCUUACCAGAUGCUUCUGGCCCUGGCCCGUCUGGACGCAGAG
CAGCGAGCGCAGCAGAACGGUACAGAUUCUUUGGACGGACGGACUGGCACGCAGGACAAGGG
ACAGAAGCCCAACCUACUAGACCGACUGCGACAUCGCAAAAACGGCUACCGACACUUGAAAG
ACUCUGACGAAGAAGAGAACGUCUGAUAAUAGGCUGGAGCCUCGGUGGCCAUGCUUCUUGCC
CCUUGGGCCUCCCCCCAGCCCCUCCUCCCCUUCCUGCACCCGUACCCCCGUGGUCUUUGAAU
AAAGUCUGAGUGGGCGGC
hCMV gBFLAG, hCMV glycoproteinB-FLAG
(SEQ ID NO: 6)
TCAAGCTTTTGGACCCTCGTACAGAAGCTAATACGACTCACTATAGGGAAATAAGAGAGAAA
AGAAGAGTAAGAAGAAATATAAGAGCCACCATGGAATCCAGGATCTGGTGCCTGGTAGTCTG
CGTTAACTTGTGTATCGTCTGTCTGGGTGCTGCGGTTTCCTCATCTTCTACTCGTGGAACTT
CTGCTACTCACAGTCACCATTCCTCTCATACGACGTCTGCTGCTCACTCTCGATCCGGTTCA
GTCTCTCAACGCGTAACTTCTTCCCAAACGGTCAGCCATGGTGTTAACGAGACCATCTACAA
CACTACCCTCAAGTACGGAGATGTGGTGGGGGTCAATACCACCAAGTACCCCTATCGCGTGT
GTTCTATGGCCCAGGGTACGGATCTTATTCGCTTTGAACGTAATATCGTCTGCACCTCGATG
AAGCCCATCAATGAAGACCTGGACGAGGGCATCATGGTGGTCTACAAACGCAACATCGTCGC
GCACACCTTTAAGGTACGAGTCTACCAGAAGGTTTTGACGTTTCGTCGTAGCTACGCTTACA
TCCACACCACTTATCTGCTGGGCAGCAACACGGAATACGTGGCGCCTCCTATGTGGGAGATT
CATCATATCAACAGCCACAGTCAGTGCTACAGTTCCTACAGCCGCGTTATAGCAGGCACGGT
TTTCGTGGCTTATCATAGGGACAGCTATGAAAACAAAACCATGCAATTAATGCCCGACGATT
ATTCCAACACCCACAGTACCCGTTACGTGACGGTCAAGGATCAATGGCACAGCCGCGGCAGC
ACCTGGCTCTATCGTGAGACCTGTAATCTGAATTGTATGGTGACCATCACTACTGCGCGCTC
CAAATATCCTTATCATTTTTTCGCCACTTCCACGGGTGACGTGGTTGACATTTCTCCTTTCT
ACAACGGAACCAATCGCAATGCCAGCTACTTTGGAGAAAACGCCGACAAGTTTTTCATTTTT
CCGAACTACACTATCGTCTCCGACTTTGGAAGACCGAATTCTGCGTTAGAGACCCACAGGTT
GGTGGCTTTTCTTGAACGTGCGGACTCGGTGATCTCCTGGGATATACAGGACGAAAAGAATG
TCACTTGTCAACTCACTTTCTGGGAAGCCTCGGAACGCACCATTCGTTCCGAAGCCGAGGAC
TCGTATCACTTTTCTTCTGCCAAAATGACCGCCACTTTCTTATCTAAGAAGCAAGAGGTGAA
CATGTCCGACTCTGCGCTGGACTGCGTACGTGATGAGGCTATAAATAAGTTACAGCAGATTT
TCAATACTTCATACAATCAAACATATGAAAAATATGGAAACGTGTCCGTCTTTGAAACCACT
GGTGGTTTGGTAGTGTTCTGGCAAGGTATCAAGCAAAAATCTCTGGTGGAACTCGAACGTTT
GGCCAACCGCTCCAGTCTGAATCTTACTCATAATAGAACCAAAAGAAGTACAGATGGCAACA
ATGCAACTCATTTATCCAACATGGAATCGGTGCACAATCTGGTCTACGCCCAGCTGCAGTTC
ACCTATGACACGTTGCGCGGTTACATCAACCGGGCGCTGGCGCAAATCGCAGAAGCCTGGTG
TGTGGATCAACGGCGCACCCTAGAGGTCTTCAAGGAACTCAGCAAGATCAACCCGTCAGCCA
TTCTCTCGGCCATTTACAACAAACCGATTGCCGCGCGTTTCATGGGTGATGTCTTGGGCCTG
GCCAGCTGCGTGACCATCAACCAAACCAGCGTCAAGGTGCTGCGTGATATGAACGTGAAGGA
GTCGCCAGGACGCTGCTACTCACGACCCGTGGTCATCTTTAATTTCGCCAACAGCTCGTACG
TGCAGTACGGTCAACTGGGCGAGGACAACGAAATCCTGTTGGGCAACCACCGCACTGAGGAA
TGTCAGCTTCCCAGCCTCAAGATCTTCATCGCCGGGAACTCGGCCTACGAGTACGTGGACTA
CCTCTTCAAACGCATGATTGACCTCAGCAGTATCTCCACCGTCGACAGCATGATCGCCCTGG
ATATCGACCCGCTGGAAAATACCGACTTCAGGGTACTGGAACTTTACTCGCAGAAAGAGCTG
CGTTCCAGCAACGTTTTTGACCTCGAAGAGATCATGCGCGAATTCAACTCGTACAAGCAGCG
GGTAAAGTACGTGGAGGACAAGGTAGTCGACCCGCTACCGCCCTACCTCAAGGGTCTGGACG
ACCTCATGAGCGGCCTGGGCGCCGCGGGAAAGGCCGTTGGCGTAGCCATTGGGGCCGTGGGT
GGCGCGGTGGCCTCCGTGGTCGAAGGCGTTGCCACCTTCCTCAAAAACCCCTTCGGAGCGTT
CACCATCATCCTCGTGGCCATAGCTGTAGTCATTATCACTTATTTGATCTATACTCGACAGC
GGCGTTTGTGCACGCAGCCGCTGCAGAACCTCTTTCCCTATCTGGTGTCCGCCGACGGGACC
ACCGTGACGTCGGGCAGCACCAAAGACACGTCGTTACAGGCTCCGCCTTCCTACGAGGAAAG
TGTTTATAATTCTGGTCGCAAAGGACCGGGACCACCGTCGTCTGATGCATCCACGGCGGCTC
CGCCTTACACCAACGAGCAGGCTTACCAGATGCTTCTGGCCCTGGCCCGTCTGGACGCAGAG
CAGCGAGCGCAGCAGAACGGTACAGATTCTTTGGACGGACGGACTGGCACGCAGGACAAGGG
ACAGAAGCCCAACCTACTAGACCGACTGCGACATCGCAAAAACGGCTACCGACACTTGAAAG
ACTCTGACGAAGAAGAGAACGTCGATTACAAGGACGATGACGATAAGTGATAATAGGCTGGA
GCCTCGGTGGCCATGCTTCTTGCCCCTTGGGCCTCCCCCCAGCCCCTCCTCCCCTTCCTGCA
CCCGTACCCCCGTGGTCTTTGAATAAAGTCTGAGTGGGCGGC
hCMV BFLAG, hCMV glycoproteinB-FLAG mRNA
(SEQ ID NO: 163)
TCAAGCUUUUGGACCCUCGUACAGAAGCUAAUACGACUCACUAUAGGGAAAUAAGAGAGAAA
AGAAGAGUAAGAAGAAAUAUAAGAGCCACCAUGGAAUCCAGGAUCUGGUGCCUGGUAGUCUG
CGUUAACUUGUGUAUCGUCUGUCUGGGUGCUGCGGUUUCCUCAUCUUCUACUCGUGGAACUU
CUGCUACUCACAGUCACCAUUCCUCUCAUACGACGUCUGCUGCUCACUCUCGAUCCGGUUCA
GUCUCUCAACGCGUAACUUCUUCCCAAACGGUCAGCCAUGGUGUUAACGAGACCAUCUACAA
CACUACCCUCAAGUACGGAGAUGUGGUGGGGGUCAAUACCACCAAGUACCCCUAUCGCGUGU
GUUCUAUGGCCCAGGGUACGGAUCUUAUUCGCUUUGAACGUAAUAUCGUCUGCACCUCGAUG
AAGCCCAUCAAUGAAGACCUGGACGAGGGCAUCAUGGUGGUCUACAAACGCAACAUCGUCGC
GCACACCUUUAAGGUACGAGUCUACCAGAAGGUUUUGACGUUUCGUCGUAGCUACGCUUACA
UCCACACCACUUAUCUGCUGGGCAGCAACACGGAAUACGUGGCGCCUCCUAUGUGGGAGAUU
CAUCAUAUCAACAGCCACAGUCAGUGCUACAGUUCCUACAGCCGCGUUAUAGCAGGCACGGU
UUUCGUGGCUUAUCAUAGGGACAGCUAUGAAAACAAAACCAUGCAAUUAAUGCCCGACGAUU
AUUCCAACACCCACAGUACCCGUUACGUGACGGUCAAGGAUCAAUGGCACAGCCGCGGCAGC
ACCUGGCUCUAUCGUGAGACCUGUAAUCUGAAUUGUAUGGUGACCAUCACUACUGCGCGCUC
CAAAUAUCCUUAUCAUUUUUUCGCCACUUCCACGGGUGACGUGGUUGACAUUUCUCCUUUCU
ACAACGGAACCAAUCGCAAUGCCAGCUACUUUGGAGAAAACGCCGACAAGUUUUUCAUUUUU
CCGAACUACACUAUCGUCUCCGACUUUGGAAGACCGAAUUCUGCGUUAGAGACCCACAGGUU
GGUGGCUUUUCUUGAACGUGCGGACUCGGUGAUCUCCUGGGAUAUACAGGACGAAAAGAAUG
UCACUUGUCAACUCACUUUCUGGGAAGCCUCGGAACGCACCAUUCGUUCCGAAGCCGAGGAC
UCGUAUCACUUUUCUUCUGCCAAAAUGACCGCCACUUUCUUAUCUAAGAAGCAAGAGGUGAA
CAUGUCCGACUCUGCGCUGGACUGCGUACGUGAUGAGGCUAUAAAUAAGUUACAGCAGAUUU
UCAAUACUUCAUACAAUCAAACAUAUGAAAAAUAUGGAAACGUGUCCGUCUUUGAAACCACU
GGUGGUUUGGUAGUGUUCUGGCAAGGUAUCAAGCAAAAAUCUCUGGUGGAACUCGAACGUUU
GGCCAACCGCUCCAGUCUGAAUCUUACUCAUAAUAGAACCAAAAGAAGUACAGAUGGCAACA
AUGCAACUCAUUUAUCCAACAUGGAAUCGGUGCACAAUCUGGUCUACGCCCAGCUGCAGUUC
ACCUAUGACACGUUGCGCGGUUACAUCAACCGGGCGCUGGCGCAAAUCGCAGAAGCCUGGUG
UGUGGAUCAACGGCGCACCCUAGAGGUCUUCAAGGAACUCAGCAAGAUCAACCCGUCAGCCA
UUCUCUCGGCCAUUUACAACAAACCGAUUGCCGCGCGUUUCAUGGGUGAUGUCUUGGGCCUG
GCCAGCUGCGUGACCAUCAACCAAACCAGCGUCAAGGUGCUGCGUGAUAUGAACGUGAAGGA
GUCGCCAGGACGCUGCUACUCACGACCCGUGGUCAUCUUUAAUUUCGCCAACAGCUCGUACG
UGCAGUACGGUCAACUGGGCGAGGACAACGAAAUCCUGUUGGGCAACCACCGCACUGAGGAA
UGUCAGCUUCCCAGCCUCAAGAUCUUCAUCGCCGGGAACUCGGCCUACGAGUACGUGGACUA
CCUCUUCAAACGCAUGAUUGACCUCAGCAGUAUCUCCACCGUCGACAGCAUGAUCGCCCUGG
AUAUCGACCCGCUGGAAAAUACCGACUUCAGGGUACUGGAACUUUACUCGCAGAAAGAGCUG
CGUUCCAGCAACGUUUUUGACCUCGAAGAGAUCAUGCGCGAAUUCAACUCGUACAAGCAGCG
GGUAAAGUACGUGGAGGACAAGGUAGUCGACCCGCUACCGCCCUACCUCAAGGGUCUGGACG
ACCUCAUGAGCGGCCUGGGCGCCGCGGGAAAGGCCGUUGGCGUAGCCAUUGGGGCCGUGGGU
GGCGCGGUGGCCUCCGUGGUCGAAGGCGUUGCCACCUUCCUCAAAAACCCCUUCGGAGCGUU
CACCAUCAUCCUCGUGGCCAUAGCUGUAGUCAUUAUCACUUAUUUGAUCUAUACUCGACAGC
GGCGUUUGUGCACGCAGCCGCUGCAGAACCUCUUUCCCUAUCUGGUGUCCGCCGACGGGACC
ACCGUGACGUCGGGCAGCACCAAAGACACGUCGUUACAGGCUCCGCCUUCCUACGAGGAAAG
UGUUUAUAAUUCUGGUCGCAAAGGACCGGGACCACCGUCGUCUGAUGCAUCCACGGCGGCUC
CGCCUUACACCAACGAGCAGGCUUACCAGAUGCUUCUGGCCCUGGCCCGUCUGGACGCAGAG
CAGCGAGCGCAGCAGAACGGUACAGAUUCUUUGGACGGACGGACUGGCACGCAGGACAAGGG
ACAGAAGCCCAACCUACUAGACCGACUGCGACAUCGCAAAAACGGCUACCGACACUUGAAAG
ACUCUGACGAAGAAGAGAACGUCGAUUACAAGGACGAUGACGAUAAGUGAUAAUAGGCUGGA
GCCUCGGUGGCCAUGCUUCUUGCCCCUUGGGCCUCCCCCCAGCCCCUCCUCCCCUUCCUGCA
CCCGUACCCCCGUGGUCUUUGAAUAAAGUCUGAGUGGGCGGC

### Example 13: hCMV Vaccine—hCMV Variant Glycoprotein Sequences

A hCMV vaccine may comprise, for example, at least one RNA polynucleotide encoded by at least one of the following sequences or by at least one fragment or epitope of the following sequences. In some embodiments, a hCMV vaccine may comprise at least one RNA polynucleotide comprising at least one of the mRNA sequences listed below or at least one fragment of the mRNA sequences listed below.

hCMV-gHtrunc, hCMV glycoproteinH (Ectodomain)
(SEQ ID NO: 7)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCCAGGCCTCCCCTCCTACCTCATCAT
CCTCGCCGTCTGTCTCTTCAGCCACCTACTTTCGTCACGATATGGCGCAGAAGCCGTATCCG
AACCGCTGGACAAAGCGTTTCACCTACTGCTCAACACCTACGGGAGACCCATCCGCTTCCTG
CGTGAAAATACCACCCAGTGTACCTACAACAGCAGCCTCCGTAACAGCACGGTCGTCAGGGA
AAACGCCATCAGTTTCAACTTTTTCCAAAGCTATAATCAATACTATGTATTCCATATGCCTC
GATGTCTTTTTGCGGGTCCTCTGGCGGAGCAGTTTCTGAACCAGGTAGATCTGACCGAAACC
CTGGAAAGATACCAACAGAGACTTAACACTTACGCGCTGGTATCCAAAGACCTGGCCAGCTA
CCGATCTTTTTCGCAGCAGCTAAAGGCACAAGACAGCCTAGGTGAACAGCCCACCACTGTGC
CACCGCCCATTGACCTGTCAATACCTCACGTTTGGATGCCACCGCAAACCACTCCACACGGC
TGGACAGAATCACATACCACCTCAGGACTACACCGACCACACTTTAACCAGACCTGTATCCT
CTTTGATGGACACGATCTACTATTCAGCACCGTCACACCTTGTTTGCACCAAGGCTTTTACC
TCATCGACGAACTACGTTACGTTAAAATAACACTGACCGAGGACTTCTTCGTAGTTACGGTG
TCCATAGACGACGACACACCCATGCTGCTTATCTTCGGCCATCTTCCACGCGTACTTTTCAA
AGCGCCCTATCAACGCGACAACTTTATACTACGACAAACTGAAAAACACGAGCTCCTGGTGC
TAGTTAAGAAAGATCAACTGAACCGTCACTCTTATCTCAAAGACCCGGACTTTCTTGACGCC
GCACTTGACTTCAACTACCTAGACCTCAGCGCACTACTACGTAACAGCTTTCACCGTTACGC
CGTGGATGTACTCAAGAGCGGTCGATGTCAGATGCTGGACCGCCGCACGGTAGAAATGGCCT
TCGCCTACGCATTAGCACTGTTCGCAGCAGCCCGACAAGAAGAGGCCGGCGCCCAAGTCTCC
GTCCCACGGGCCCTAGACCGCCAGGCCGCACTCTTACAAATACAAGAATTTATGATCACCTG
CCTCTCACAAACACCACCACGCACCACGTTGCTGCTGTATCCCACGGCCGTGGACCTGGCCA
AACGAGCCCTTTGGACACCGAATCAGATCACCGACATCACCAGCCTCGTACGCCTGGTCTAC
ATACTCTCTAAACAGAATCAGCAACATCTCATCCCCCAATGGGCACTACGACAGATCGCCGA
CTTTGCCCTAAAACTACACAAAACGCACCTGGCCTCTTTTCTTTCAGCCTTCGCACGCCAAG
AACTCTACCTCATGGGCAGCCTCGTCCACTCCATGCTGGTACATACGACGGAGAGACGCGAA
ATCTTCATCGTAGAAACGGGCCTCTGTTCATTGGCCGAGCTATCACACTTTACGCAGTTGTT
AGCTCATCCACACCACGAATACCTCAGCGACCTGTACACACCCTGTTCCAGTAGCGGGCGAC
GCGATCACTCGCTCGAACGCCTCACGCGTCTCTTCCCCGATGCCACCGTCCCCGCTACCGTT
CCCGCCGCCCTCTCCATCCTATCTACCATGCAACCAAGCACGCTGGAAACCTTCCCCGACCT
GTTTTGCTTGCCGCTCGGCGAATCCTTCTCCGCGCTGACCGTCTCCGAACACGTCAGTTATA
TCGTAACAAACCAGTACCTGATCAAAGGTATCTCCTACCCTGTCTCCACCACCGTCGTAGGC
CAGAGCCTCATCATCACCCAGACGGACAGTCAAACTAAATGCGAACTGACGCGCAACATGCA
TACCACACACAGCATCACAGTGGCGCTCAACATTTCGCTAGAAAACTGCGCCTTTTGCCAAA
GCGCCCTGCTAGAATACGACGACACGCAAGGCGTCATCAACATCATGTACATGCACGACTCG
GACGACGTCCTTTTCGCCCTGGATCCCTACAACGAAGTGGTGGTCTCATCTCCGCGAACTCA
CTACCTCATGCTTTTGAAAAACGGTACGGTACTAGAAGTAACTGACGTCGTCGTGGACGCCA
CCGACTGATAATAGGCTGGAGCCTCGGTGGCCATGCTTCTTGCCCCTTGGGCCTCCCCCCAG
CCCCTCCTCCCCTTCCTGCACCCGTACCCCCGTGGTCTTTGAATAAAGTCTGAGTGGGCGGC
hCMV-gHtrune, hCMV glycoproteinH (Ectodomain) mRNA
(SEQ ID NO: 164)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCCAGGCCUCCCCUCCUACCUCAUCAU
CCUCGCCGUCUGUCUCUUCAGCCACCUACUUUCGUCACGAUAUGGCGCAGAAGCCGUAUCCG
AACCGCUGGACAAAGCGUUUCACCUACUGCUCAACACCUACGGGAGACCCAUCCGCUUCCUG
CGUGAAAAUACCACCCAGUGUACCUACAACAGCAGCCUCCGUAACAGCACGGUCGUCAGGGA
AAACGCCAUCAGUUUCAACUUUUUCCAAAGCUAUAAUCAAUACUAUGUAUUCCAUAUGCCUC
GAUGUCUUUUUGCGGGUCCUCUGGCGGAGCAGUUUCUGAACCAGGUAGAUCUGACCGAAACC
CUGGAAAGAUACCAACAGAGACUUAACACUUACGCGCUGGUAUCCAAAGACCUGGCCAGCUA
CCGAUCUUUUUCGCAGCAGCUAAAGGCACAAGACAGCCUAGGUGAACAGCCCACCACUGUGC
CACCGCCCAUUGACCUGUCAAUACCUCACGUUUGGAUGCCACCGCAAACCACUCCACACGGC
UGGACAGAAUCACAUACCACCUCAGGACUACACCGACCACACUUUAACCAGACCUGUAUCCU
CUUUGAUGGACACGAUCUACUAUUCAGCACCGUCACACCUUGUUUGCACCAAGGCUUUUACC
UCAUCGACGAACUACGUUACGUUAAAAUAACACUGACCGAGGACUUCUUCGUAGUUACGGUG
UCCAUAGACGACGACACACCCAUGCUGCUUAUCUUCGGCCAUCUUCCACGCGUACUUUUCAA
AGCGCCCUAUCAACGCGACAACUUUAUACUACGACAAACUGAAAAACACGAGCUCCUGGUGC
UAGUUAAGAAAGAUCAACUGAACCGUCACUCUUAUCUCAAAGACCCGGACUUUCUUGACGCC
GCACUUGACUUCAACUACCUAGACCUCAGCGCACUACUACGUAACAGCUUUCACCGUUACGC
CGUGGAUGUACUCAAGAGCGGUCGAUGUCAGAUGCUGGACCGCCGCACGGUAGAAAUGGCCU
UCGCCUACGCAUUAGCACUGUUCGCAGCAGCCCGACAAGAAGAGGCCGGCGCCCAAGUCUCC
GUCCCACGGGCCCUAGACCGCCAGGCCGCACUCUUACAAAUACAAGAAUUUAUGAUCACCUG
CCUCUCACAAACACCACCACGCACCACGUUGCUGCUGUAUCCCACGGCCGUGGACCUGGCCA
AACGAGCCCUUUGGACACCGAAUCAGAUCACCGACAUCACCAGCCUCGUACGCCUGGUCUAC
AUACUCUCUAAACAGAAUCAGCAACAUCUCAUCCCCCAAUGGGCACUACGACAGAUCGCCGA
CUUUGCCCUAAAACUACACAAAACGCACCUGGCCUCUUUUCUUUCAGCCUUCGCACGCCAAG
AACUCUACCUCAUGGGCAGCCUCGUCCACUCCAUGCUGGUACAUACGACGGAGAGACGCGAA
AUCUUCAUCGUAGAAACGGGCCUCUGUUCAUUGGCCGAGCUAUCACACUUUACGCAGUUGUU
AGCUCAUCCACACCACGAAUACCUCAGCGACCUGUACACACCCUGUUCCAGUAGCGGGCGAC
GCGAUCACUCGCUCGAACGCCUCACGCGUCUCUUCCCCGAUGCCACCGUCCCCGCUACCGUU
CCCGCCGCCCUCUCCAUCCUAUCUACCAUGCAACCAAGCACGCUGGAAACCUUCCCCGACCU
GUUUUGCUUGCCGCUCGGCGAAUCCUUCUCCGCGCUGACCGUCUCCGAACACGUCAGUUAUA
UCGUAACAAACCAGUACCUGAUCAAAGGUAUCUCCUACCCUGUCUCCACCACCGUCGUAGGC
CAGAGCCUCAUCAUCACCCAGACGGACAGUCAAACUAAAUGCGAACUGACGCGCAACAUGCA
UACCACACACAGCAUCACAGUGGCGCUCAACAUUUCGCUAGAAAACUGCGCCUUUUGCCAAA
GCGCCCUGCUAGAAUACGACGACACGCAAGGCGUCAUCAACAUCAUGUACAUGCACGACUCG
GACGACGUCCUUUUCGCCCUGGAUCCCUACAACGAAGUGGUGGUCUCAUCUCCGCGAACUCA
CUACCUCAUGCUUUUGAAAAACGGUACGGUACUAGAAGUAACUGACGUCGUCGUGGACGCCA
CCGACUGAUAAUAGGCUGGAGCCUCGGUGGCCAUGCUUCUUGCCCCUUGGGCCUCCCCCCAG
CCCCUCCUCCCCUUCCUGCACCCGUACCCCCGUGGUCUUUGAAUAAAGUCUGAGUGGGCGGC
hCMV-gHtruncFLAG, glycoprotein H Ectodomain
(SEQ ID NO: 8)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCCAGGCCTCCCCTCCTACCTCATCAT
CCTCGCCGTCTGTCTCTTCAGCCACCTACTTTCGTCACGATATGGCGCAGAAGCCGTATCCG
AACCGCTGGACAAAGCGTTTCACCTACTGCTCAACACCTACGGGAGACCCATCCGCTTCCTG
CGTGAAAATACCACCCAGTGTACCTACAACAGCAGCCTCCGTAACAGCACGGTCGTCAGGGA
AAACGCCATCAGTTTCAACTTTTTCCAAAGCTATAATCAATACTATGTATTCCATATGCCTC
GATGTCTTTTTGCGGGTCCTCTGGCGGAGCAGTTTCTGAACCAGGTAGATCTGACCGAAACC
CTGGAAAGATACCAACAGAGACTTAACACTTACGCGCTGGTATCCAAAGACCTGGCCAGCTA
CCGATCTTTTTCGCAGCAGCTAAAGGCACAAGACAGCCTAGGTGAACAGCCCACCACTGTGC
CACCGCCCATTGACCTGTCAATACCTCACGTTTGGATGCCACCGCAAACCACTCCACACGGC
TGGACAGAATCACATACCACCTCAGGACTACACCGACCACACTTTAACCAGACCTGTATCCT
CTTTGATGGACACGATCTACTATTCAGCACCGTCACACCTTGTTTGCACCAAGGCTTTTACC
TCATCGACGAACTACGTTACGTTAAAATAACACTGACCGAGGACTTCTTCGTAGTTACGGTG
TCCATAGACGACGACACACCCATGCTGCTTATCTTCGGCCATCTTCCACGCGTACTTTTCAA
AGCGCCCTATCAACGCGACAACTTTATACTACGACAAACTGAAAAACACGAGCTCCTGGTGC
TAGTTAAGAAAGATCAACTGAACCGTCACTCTTATCTCAAAGACCCGGACTTTCTTGACGCC
GCACTTGACTTCAACTACCTAGACCTCAGCGCACTACTACGTAACAGCTTTCACCGTTACGC
CGTGGATGTACTCAAGAGCGGTCGATGTCAGATGCTGGACCGCCGCACGGTAGAAATGGCCT
TCGCCTACGCATTAGCACTGTTCGCAGCAGCCCGACAAGAAGAGGCCGGCGCCCAAGTCTCC
GTCCCACGGGOCCTAGACCGCCAGGCCGCACTCTTACAAATACAAGAATTTATGATCACCTG
CCTCTCACAAACACCACCACGCACCACGTTGCTGCTGTATCCCACGGCCGTGGACCTGGCCA
AACGAGCCCTTTGGACACCGAATCAGATCACCGACATCACCAGCCTCGTACGCCTGGTCTAC
ATACTCTCTAAACAGAATCAGCAACATCTCATCCCCCAATGGGCACTACGACAGATCGCCGA
CTTTGCCCTAAAACTACACAAAACGCACCTGGCCTCTTTTCTTTCAGCCTTCGCACGCCAAG
AACTCTACCTCATGGGCAGCCTCGTCCACTCCATGCTGGTACATACGACGGAGAGACGCGAA
ATCTTCATCGTAGAAACGGGCCTCTGTTCATTGGCCGAGCTATCACACTTTACGCAGTTGTT
AGCTCATCCACACCACGAATACCTCAGCGACCTGTACACACCCTGTTCCAGTAGCGGGCGAC
GCGATCACTCGCTCGAACGCCTCACGCGTCTCTTCCCCGATGCCACCGTCCCCGCTACCGTT
CCCGCCGCCCTCTCCATCCTATCTACCATGCAACCAAGCACGCTGGAAACCTTCCCCGACCT
GTTTTGCTTGCCGCTCGGCGAATCCTTCTCCGCGCTGACCGTCTCCGAACACGTCAGTTATA
TCGTAACAAACCAGTACCTGATCAAAGGTATCTCCTACCCTGTCTCCACCACCGTCGTAGGC
CAGAGCCTCATCATCACCCAGACGGACAGTCAAACTAAATGCGAACTGACGCGCAACATGCA
TACCACACACAGCATCACAGTGGCGCTCAACATTTCGCTAGAAAACTGCGCCTTTTGCCAAA
GCGCCCTGCTAGAATACGACGACACGCAAGGCGTCATCAACATCATGTACATGCACGACTCG
GACGACGTccTTTTCGCCCTGGATCCCTACAACGAAGTGGTGGTCTCATCTCCGCGAACTCA
CTACCTCATGCTTTTGAAAAACGGTACGGTACTAGAAGTAACTGACGTCGTCGTGGACGCCA
CCGACGATTACAAGGACGATGACGATAAGTGATGATAATAGGCTGGAGCCTCGGTGGCCATG
CTTCTTGCCCCTTGGGCCTCCCCCCAGCCCCTCCTCCCCTTCCTGCACCCGTACCCCCGTGG
TCTTTGAATAAAGTCTGAGTGGGCGG
hCMV-gHtruncFLAG, glycoprotein H Ectodomain mRNA
(SEQ ID NO: 165)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCCAGGCCUCCCCUCCUACCUCAUCAU
CCUCGCCGUCUGUCUCUUCAGCCACCUACUUUCGUCACGAUAUGGCGCAGAAGCCGUAUCCG
AACCGCUGGACAAAGCGUUUCACCUACUGCUCAACACCUACGGGAGACCCAUCCGCUUCCUG
CGUGAAAAUACCACCCAGUGUACCUACAACAGCAGCCUCCGUAACAGCACGGUCGUCAGGGA
AAACGCCAUCAGUUUCAACUUUUUCCAAAGCUAUAAUCAAUACUAUGUAUUCCAUAUGCCUC
GAUGUCUUUUUGCGGGUCCUCUGGCGGAGCAGUUUCUGAACCAGGUAGAUCUGACCGAAACC
CUGGAAAGAUACCAACAGAGACUUAACACUUACGCGCUGGUAUCCAAAGACCUGGCCAGCUA
CCGAUCUUUUUCGCAGCAGCUAAAGGCACAAGACAGCCUAGGUGAACAGCCCACCACUGUGC
CACCGCCCAUUGACCUGUCAAUACCUCACGUUUGGAUGCCACCGCAAACCACUCCACACGGC
UGGACAGAAUCACAUACCACCUCAGGACUACACCGACCACACUUUAACCAGACCUGUAUCCU
CUUUGAUGGACACGAUCUACUAUUCAGCACCGUCACACCUUGUUUGCACCAAGGCUUUUACC
UCAUCGACGAACUACGUUACGUUAAAAUAACACUGACCGAGGACUUCUUCGUAGUUACGGUG
UCCAUAGACGACGACACACCCAUGCUGCUUAUCUUCGGCCAUCUUCCACGCGUACUUUUCAA
AGCGCCCUAUCAACGCGACAACUUUAUACUACGACAAACUGAAAAACACGAGCUCCUGGUGC
UAGUUAAGAAAGAUCAACUGAACCGUCACUCUUAUCUCAAAGACCCGGACUUUCUUGACGCC
GCACUUGACUUCAACUACCUAGACCUCAGCGCACUACUACGUAACAGCUUUCACCGUUACGC
CGUGGAUGUACUCAAGAGCGGUCGAUGUCAGAUGCUGGACCGCCGCACGGUAGAAAUGGCCU
UCGCCUACGCAUUAGCACUGUUCGCAGCAGCCCGACAAGAAGAGGCCGGCGCCCAAGUCUCC
GUCCCACGGGCCCUAGACCGCCAGGCCGCACUCUUACAAAUACAAGAAUUUAUGAUCACCUG
CCUCUCACAAACACCACCACGCACCACGUUGCUGCUGUAUCCCACGGCCGUGGACCUGGCCA
AACGAGCCCUUUGGACACCGAAUCAGAUCACCGACAUCACCAGCCUCGUACGCCUGGUCUAC
AUACUCUCUAAACAGAAUCAGCAACAUCUCAUCCCCCAAUGGGCACUACGACAGAUCGCCGA
CUUUGCCCUAAAACUACACAAAACGCACCUGGCCUCUUUUCUUUCAGCCUUCGCACGCCAAG
AACUCUACCUCAUGGGCAGCCUCGUCCACUCCAUGCUGGUACAUACGACGGAGAGACGCGAA
AUCUUCAUCGUAGAAACGGGCCUCUGUUCAUUGGCCGAGCUAUCACACUUUACGCAGUUGUU
AGCUCAUCCACACCACGAAUACCUCAGCGACCUGUACACACCCUGUUCCAGUAGCGGGCGAC
GCGAUCACUCGCUCGAACGCCUCACGCGUCUCUUCCCCGAUGCCACCGUCCCCGCUACCGUU
CCCGCCGCCCUCUCCAUCCUAUCUACCAUGCAACCAAGCACGCUGGAAACCUUCCCCGACCU
GUUUUGCUUGCCGCUCGGCGAAUCCUUCUCCGCGCUGACCGUCUCCGAACACGUCAGUUAUA
UCGUAACAAACCAGUACCUGAUCAAAGGUAUCUCCUACCCUGUCUCCACCACCGUCGUAGGC
CAGAGCCUCAUCAUCACCCAGACGGACAGUCAAACUAAAUGCGAACUGACGCGCAACAUGCA
UACCACACACAGCAUCACAGUGGCGCUCAACAUUUCGCUAGAAAACUGCGCCUUUUGCCAAA
GCGCCCUGCUAGAAUACGACGACACGCAAGGCGUCAUCAACAUCAUGUACAUGCACGACUCG
GACGACGUCCUUUUCGCCCUGGAUCCCUACAACGAAGUGGUGGUCUCAUCUCCGCGAACUCA
CUACCUCAUGCUUUUGAAAAACGGUACGGUACUAGAAGUAACUGACGUCGUCGUGGACGCCA
CCGACGAUUACAAGGACGAUGACGAUAAGUGAUGAUAAUAGGCUGGAGCCUCGGUGGCCAUG
CUUCUUGCCCCUUGGGCCUCCCCCCAGCCCCUCCUCCCCUUCCUGCACCCGUACCCCCGUGG
UCUUUGAAUAAAGUCUGAGUGGGCGGC
hCMVgHtrunc6XHis, glycoprotein H Ectodomain-6XHis tag
(SEQ ID NO: 9)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCCAGGCCTCCCCTCCTACCTCATCAT
CCTCGCCGTCTGTCTCTTCAGCCACCTACTTTCGTCACGATATGGCGCAGAAGCCGTATCCG
AACCGCTGGACAAAGCGTTTCACCTACTGCTCAACACCTACGGGAGACCCATCCGCTTCCTG
CGTGAAAATACCACCCAGTGTACCTACAACAGCAGCCTCCGTAACAGCACGGTCGTCAGGGA
AAACGCCATCAGTTTCAACTTTTTCCAAAGCTATAATCAATACTATGTATTCCATATGCCTC
GATGTCTTTTTGCGGGTCCTCTGGCGGAGCAGTTTCTGAACCAGGTAGATCTGACCGAAACC
CTGGAAAGATACCAACAGAGACTTAACACTTACGCGCTGGTATCCAAAGACCTGGCCAGCTA
CCGATCTTTTTCGCAGCAGCTAAAGGCACAAGACAGCCTAGGTGAACAGCCCACCACTGTGC
CACCGCCCATTGACCTGTCAATACCTCACGTTTGGATGCCACCGCAAACCACTCCACACGGC
TGGACAGAATCACATACCACCTCAGGACTACACCGACCACACTTTAACCAGACCTGTATCCT
CTTTGATGGACACGATCTACTATTCAGCACCGTCACACCTTGTTTGCACCAAGGCTTTTACC
TCATCGACGAACTACGTTACGTTAAAATAACACTGACCGAGGACTTCTTCGTAGTTACGGTG
TCCATAGACGACGACACACCCATGCTGCTTATCTTCGGCCATCTTCCACGCGTACTTTTCAA
AGCGCCCTATCAACGCGACAACTTTATACTACGACAAACTGAAAAACACGAGCTCCTGGTGC
TAGTTAAGAAAGATCAACTGAACCGTCACTCTTATCTCAAAGACCCGGACTTTCTTGACGCC
GCACTTGACTTCAACTACCTAGACCTCAGCGCACTACTACGTAACAGCTTTCACCGTTACGC
CGTGGATGTACTCAAGAGCGGTCGATGTCAGATGCTGGACCGCCGCACGGTAGAAATGGCCT
TCGCCTACGCATTAGCACTGTTCGCAGCAGCCCGACAAGAAGAGGCCGGCGCCCAAGTCTCC
GTCCCACGGGCCCTAGACCGCCAGGCCGCACTCTTACAAATACAAGAATTTATGATCACCTG
CCTCTCACAAACACCACCACGCACCACGTTGCTGCTGTATCCCACGGCCGTGGACCTGGCCA
AACGAGCCCTTTGGACACCGAATCAGATCACCGACATCACCAGCCTCGTACGCCTGGTCTAC
ATACTCTCTAAACAGAATCAGCAACATCTCATCCCCCAATGGGCACTACGACAGATCGCCGA
CTTTGCCCTAAAACTACACAAAACGCACCTGGCCTCTTTTCTTTCAGCCTTCGCACGCCAAG
AACTCTACCTCATGGGCAGCCTCGTCCACTCCATGCTGGTACATACGACGGAGAGACGCGAA
ATCTTCATCGTAGAAACGGGCCTCTGTTCATTGGCCGAGCTATCACACTTTACGCAGTTGTT
AGCTCATCCACACCACGAATACCTCAGCGACCTGTACACACCCTGTTCCAGTAGCGGGCGAC
GCGATCACTCGCTCGAACGCCTCACGCGTCTCTTCCCCGATGCCACCGTCCCCGCTACCGTT
CCCGCCGCCCTCTCCATCCTATCTACCATGCAACCAAGCACGCTGGAAACCTTCCCCGACCT
GTTTTGCTTGCCGCTCGGCGAATCCTTCTCCGCGCTGACCGTCTCCGAACACGTCAGTTATA
TCGTAACAAACCAGTACCTGATCAAAGGTATCTCCTACCCTGTCTCCACCACCGTCGTAGGC
CAGAGCCTCATCATCACCCAGACGGACAGTCAAACTAAATGCGAACTGACGCGCAACATGCA
TACCACACACAGCATCACAGTGGCGCTCAACATTTCGCTAGAAAACTGCGCCTTTTGCCAAA
GCGCCCTGCTAGAATACGACGACACGCAAGGCGTCATCAACATCATGTACATGCACGACTCG
GACGACGTCCTTTTCGCCCTGGATCCCTACAACGAAGTGGTGGTCTCATCTCCGCGAACTCA
CTACCTCATGCTTTTGAAAAACGGTACGGTACTAGAAGTAACTGACGTCGTCGTGGACGCCA
CCGACCACCATCACCACCATCACTGATGATAATAGGCTGGAGCCTCGGTGGCCATGCTTCTT
GCCCCTTGGGCCTCCCCCCAGCCCCTCCTCCCCTTCCTGCACCCGTACCCCCGTGGTCTTTG
AATAAAGTCTGAGTGGGCGGC
hCMVgHtruncXHis, glycoprotein H Ectodomain-6XHis tag mRNA
(SEQ ID NO: 166)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCCAGGCCUCCCCUCCUACCUCAUCAU
CCUCGCCGUCUGUCUCUUCAGCCACCUACUUUCGUCACGAUAUGGCGCAGAAGCCGUAUCCG
AACCGCUGGACAAAGCGUUUCACCUACUGCUCAACACCUACGGGAGACCCAUCCGCUUCCUG
CGUGAAAAUACCACCCAGUGUACCUACAACAGCAGCCUCCGUAACAGUACGGUCGUCAGGGA
AAACGCCAUCAGUUUCAACUUUUUCCAAAGCUAUAAUCAAUACUAUGUAUUCCAUAUGCCUC
GAUGUCUUUUUGCGGGUCCUCUGGCGGAGCAGUUUCUGAACCAGGUAGAUCUGACCGAAACC
CUGGAAAGAUACCAACAGAGACUUAACACUUACGCGCUGGUAUCCAAAGACCUGGCCAGCUA
CCGAUCUUUUUCGCAGCAGCUAAAGGCACAAGACAGCCUAGGUGAACAGCCCACCACUGUGC
CACCGCCCAUUGACCUGUCAAUACCUCACGUUUGGAUGCCACCGCAAACCACUCCACACGGC
UGGACAGAAUCACAUACCACCUCAGGACUACACCGACCACACUUUAACCAGACCUGUAUCCU
CUUUGAUGGACACGAUCUACUAUUCAGCACCGUCACACCUUGUUUGCACCAAGGCUUUUACC
UCAUCGACGAACUACGUUACGUUAAAAUAACACUGACCGAGGACUUCUUCGUAGUUACGGUG
UCCAUAGACGACGACACACCCAUGCUGCUUAUCUUCGGCCAUCUUCCACGCGUACUUUUCAA
AGCGCCCUAUCAACGCGACAACUUUAUACUACGACAAACUGAAAAACACGAGCUCCUGGUGC
UAGUUAAGAAAGAUCAACUGAACCGUCACUCUUAUCUCAAAGACCCGGACUUUCUUGACGCC
GCACUUGACUUCAACUACCUAGACCUCAGCGCACUACUACGUAACAGCUUUCACCGUUACGC
CGUGGAUGUACUCAAGAGCGGUCGAUGUCAGAUGCUGGACUGLCGCACGGUAGAAAUGGCCU
UCGCCUACGCAUUAGCACUGUUCGCAGCAGCCCGACAAGAAGAGGCCGGCGCCCAAGUCUCC
GUCCCACGGGCCCUAGACCGCCAGGCCGCACUCUUACAAAUACAAGAAUUUAUGAUCACCUG
CCUCUCACAAACACCACCACGCACCACGUUGCUGCUGUAUCCCACGGCCGUGGACCUGGCCA
AACGAGCCCUUUGGACACCGAAUCAGAUCACCGACAUCACCAGCCUCGUACGCCUGGUCUAC
AUACUCUCUAAACAGAAUCAGCAACAUCUCAUCCCCCAAUGGGCACUACGACAGAUCGCCGA
CUUUGCCCUAAAACUACACAAAACGCACCUGGCCUCUUUUCUUUCAGCCUUCGCACGCCAAG
AACUCUACCUCAUGGGCAGCCUCGUCCACUCCAUGCUGGUACAUACGACGGAGAGACGCGAA
AUCUUCAUCGUAGAAACGGGCCUCUGUUCAUUGGCCGAGCUAUCACACUUUACGCAGUUGUU
AGCUCAUCCACACCACGAAUACCUCAGCGACCUGUACACACCCUGUUCCAGUAGCGGGCGAC
GCGAUCACUCGCUCGAACGCCUCACGCGUCUCUUCCCCGAUGCCACCGUCCCCGCUACCGUU
CCCGCCGCCCUCUCCAUCCUAUCUACCAUGCAACCAAGCACGCUGGAAACCUUCCCCGACCU
GUUUUGCUUGCCGCUCGGCGAAUCCUUCUCCGCGCUGACCGUCUCCGAACACGUCAGUUAUA
UCGUAACAAACCAGUACCUGAUCAAAGGUAUCUCCUACCCUGUCUCCACCACCGUCGUAGGC
CAGAGCCUCAUCAUCACCCAGACGGACAGUCAAACUAAAUGCGAACUGACGCGCAACAUGCA
UACCACACACAGCAUCACAGUGGCGCUCAACAUUUCGCUAGAAAACUGCGCCUUUUGCCAAA
GCGCCCUGCUAGAAUACGACGACACGCAAGGCGUCAUCAACAUCAUGUACAUGCACGACUCG
GACGACGUCCUUUUCGCCCUGGAUCCCUACAACGAAGUGGUGGUCUCAUCUCCGCGAACUCA
CUACCUCAUGCUUUUGAAAAACGGUACGGUACUAGAAGUAACUGACGUCGUCGUGGACGCCA
CCGACCACCAUCACCACCAUCACUGAUGAUAAUAGGCUGGAGCCUCGGUGGCCAUGCUUCUU
GCCCCUUGGGCCUCCCCCCAGCCCCUCCUCCCCUUCCUGCACCCGUACCCCCGUGGUCUUUG
AAUAAAGUCUGAGUGGGCGGC
hCMV TrgB,glycoprotein B (ectodomain)
(SEQ ID NO: 10)
TCAAGCTTTTGGACCCTCGTACAGAAGCTAATACGACTCACTATAGGGAAATAAGAGAGAAA
AGAAGAGTAAGAAGAAATATAAGAGCCACCATGGAATCCAGGATCTGGTGCCTGGTAGTCTG
CGTTAACTTGTGTATCGTCTGTCTGGGTGCTGCGGTTTCCTCATCTTCTACTCGTGGAACTT
CTGCTACTCACAGTCACCATTCCTCTCATACGACGTCTGCTGCTCACTCTCGATCCGGTTCA
GTCTCTCAACGCGTAACTTCTTCCCAAACGGTCAGCCATGGTGTTAACGAGACCATCTACAA
CACTACCCTCAAGTACGGAGATGTGGTGGGGGTCAATACCACCAAGTACCCCTATCGCGTGT
GTTCTATGGCCCAGGGTACGGATCTTATTCGCTTTGAACGTAATATCGTCTGCACCTCGATG
AAGCCCATCAATGAAGACCTGGACGAGGGCATCATGGTGGTCTACAAACGCAACATCGTCGC
GCACACCTTTAAGGTACGAGTCTACCAGAAGGTTTTGACGTTTCGTCGTAGCTACGCTTACA
TCCACACCACTTATCTGCTGGGCAGCAACACGGAATACGTGGCGCCTCCTATGTGGGAGATT
CATCATATCAACAGCCACAGTCAGTGCTACAGTTCCTACAGCCGCGTTATAGCAGGCACGGT
TTTCGTGGCTTATCATAGGGACAGCTATGAAAACAAAACCATGCAATTAATGCCCGACGATT
ATTCCAACACCCACAGTACCCGTTACGTGACGGTCAAGGATCAATGGCACAGCCGCGGCAGC
ACCTGGCTCTATCGTGAGACCTGTAATCTGAATTGTATGGTGACCATCACTACTGCGCGCTC
CAAATATCCTTATCATTTTTTCGCCACTTCCACGGGTGACGTGGTTGACATTTCTCCTTTCT
ACAACGGAACCAATCGCAATGCCAGCTACTTTGGAGAAAACGCCGACAAGTTTTTCATTTTT
CCGAACTACACTATCGTCTCCGACTTTGGAAGACCGAATTCTGCGTTAGAGACCCACAGGTT
GGTGGCTTTTCTTGAACGTGCGGACTCGGTGATCTCCTGGGATATACAGGACGAAAAGAATG
TCACTTGTCAACTCACTTTCTGGGAAGCCTCGGAACGCACCATTCGTTCCGAAGCCGAGGAC
TCGTATCACTTTTCTTCTGCCAAAATGACCGCCACTTTCTTATCTAAGAAGCAAGAGGTGAA
CATGTCCGACTCTGCGCTGGACTGCGTACGTGATGAGGCTATAAATAAGTTACAGCAGATTT
TCAATACTTCATACAATCAAACATATGAAAAATATGGAAACGTGTCCGTCTTTGAAACCACT
GGTGGTTTGGTAGTGTTCTGGCAAGGTATCAAGCAAAAATCTCTGGTGGAACTCGAACGTTT
GGCCAACCGCTCCAGTCTGAATCTTACTCATAATAGAACCAAAAGAAGTACAGATGGCAACA
ATGCAACTCATTTATCCAACATGGAATCGGTGCACAATCTGGTCTACGCCCAGCTGCAGTTC
ACCTATGACACGTTGCGCGGTTACATCAACCGGGCGCTGGCGCAAATCGCAGAAGCCTGGTG
TGTGGATCAACGGCGCACCCTAGAGGTCTTCAAGGAACTCAGCAAGATCAACCCGTCAGCCA
TTCTCTCGGCCATTTACAACAAACCGATTGCCGCGCGTTTCATGGGTGATGTCTTGGGCCTG
GCCAGCTGCGTGACCATCAACCAAACCAGCGTCAAGGTGCTGCGTGATATGAACGTGAAGGA
GTCGCCAGGACGCTGCTACTCACGACCCGTGGTCATCTTTAATTTCGCCAACAGCTCGTACG
TGCAGTACGGTCAACTGGGCGAGGACAACGAAATCCTGTTGGGCAACCACCGCACTGAGGAA
TGTCAGCTTCCCAGCCTCAAGATCTTCATCGCCGGGAACTCGGCCTACGAGTACGTGGACTA
CCTCTTCAAACGCATGATTGACCTCAGCAGTATCTCCACCGTCGACAGCATGATCGCCCTGG
ATATCGACCCGCTGGAAAATACCGACTTCAGGGTACTGGAACTTTACTCGCAGAAAGAGCTG
CGTTCCAGCAACGTTTTTGACCTCGAAGAGATCATGCGCGAATTCAACTCGTACAAGCAGTG
ATAATAGGCTGGAGCCTCGGTGGCCATGCTTCTTGCCCCTTGGGCCTCCCCCCAGCCCCTCC
TCCCCTTCCTGCACCCGTACCCCCGTGGTCTTTGAATAAAGTCTGAGTGGGCGGC
hCMV TrgB,glycoprotein B (ectodomain) mRNA
(SEQ ID NO: 167)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGGAAUCCAGGAUCUGGUGCCUGGUAGUCUG
CGUUAACUUGUGUAUCGUCUGUCUGGGUGCUGCGGUUUCCUCAUCUUCUACUCGUGGAACUU
CUGCUACUCACAGUCACCAUUCCUCUCAUACGACGUCUGCUGCUCACUCUCGAUCCGGUUCA
GUCUCUCAACGCGUAACUUCUUCCCAAACGGUCAGCCAUGGUGUUAACGAGACCAUCUACAA
CACUACCCUCAAGUACGGAGAUGUGGUGGGGGUCAAUACCACCAAGUACCCCUAUCGCGUGU
GUUCUAUGGCCCAGGGUACGGAUCUUAUUCGCUUUGAACGUAAUAUCGUCUGCACCUCGAUG
AAGCCCAUCAAUGAAGACCUGGACGAGGGCAUCAUGGUGGUCUACAAACGCAACAUCGUCGC
GCACACCUUUAAGGUACGAGUCUACCAGAAGGUUUUGACGUUUCGUCGUAGCUACGCUUACA
UCCACACCACUUAUCUGCUGGGCAGCAACACGGAAUACGUGGCGCCUCCUAUGUGGGAGAUU
CAUCAUAUCAACAGCCACAGUCAGUGCUACAGUUCCUACAGCCGCGUUAUAGCAGGCACGGU
UUUCGUGGCUUAUCAUAGGGACAGCUAUGAAAACAAAACCAUGCAAUUAAUGCCCGACGAUU
AUUCCAACACCCACAGUACCCGUUACGUGACGGUCAAGGAUCAAUGGCACAGCCGCGGCAGC
ACCUGGCUCUAUCGUGAGACCUGUAAUCUGAAUUGUAUGGUGACCAUCACUACUGCGCGCUC
CAAAUAUCCUUAUCAUUUUUUCGCCACUUCCACGGGUGACGUGGUUGACAUUUCUCCUUUCU
ACAACGGAACCAAUCGCAAUGCCAGCUACUUUGGAGAAAACGCCGACAAGUUUUUCAUUUUU
CCGAACUACACUAUCGUCUCCGACUUUGGAAGACCGAAUUCUGCGUUAGAGACCCACAGGUU
GGUGGCUUUUCUUGAACGUGCGGACUCGGUGAUCUCCUGGGAUAUACAGGACGAAAAGAAUG
UCACUUGUCAACUCACUUUCUGGGAAGCCUCGGAACGCACCAUUCGUUCCGAAGCCGAGGAC
UCGUAUCACUUUUCUUCUGCCAAAAUGACCGCCACUUUCUUAUCUAAGAAGCAAGAGGUGAA
CAUGUCCGACUCUGCGCUGGACUGCGUACGUGAUGAGGCUAUAAAUAAGUUACAGCAGAUUU
UCAAUACUUCAUACAAUCAAACAUAUGAAAAAUAUGGAAACGUGUCCGUCUUUGAAACCACU
GGUGGUUUGGUAGUGUUCUGGCAAGGUAUCAAGCAAAAAUCUCUGGUGGAACUCGAACGUUU
GGCCAACCGCUCCAGUCUGAAUCUUACUCAUAAUAGAACCAAAAGAAGUACAGAUGGCAACA
AUGCAACUCAUUUAUCCAACAUGGAAUCGGUGCACAAUCUGGUCUACGCCCAGCUGCAGUUC
ACCUAUGACACGUUGCGCGGUUACAUCAACCGGGCGCUGGCGCAAAUCGCAGAAGCCUGGUG
UGUGGAUCAACGGCGCACCCUAGAGGUCUUCAAGGAACUCAGCAAGAUCAACCCGUCAGCCA
UUCUCUCGGCCAUUUACAACAAACCGAUUGCCGCGCGUUUCAUGGGUGAUGUCUUGGGCCUG
GCCAGCUGCGUGACCAUCAACCAAACCAGCGUCAAGGUGCUGCGUGAUAUGAACGUGAAGGA
GUCGCCAGGACGCUGCUACUCACGACCCGUGGUCAUCUUUAAUUUCGCCAACAGCUCGUACG
UGCAGUACGGUCAACUGGGCGAGGACAACGAAAUCCUGUUGGGCAACCACCGCACUGAGGAA
UGUCAGCUUCCCAGCCUCAAGAUCUUCAUCGCCGGGAACUCGGCCUACGAGUACGUGGACUA
CCUCUUCAAACGCAUGAUUGACCUCAGCAGUAUCUCCACCGUCGACAGCAUGAUCGCCCUGG
AUAUCGACCCGCUGGAAAAUACCGACUUCAGGGUACUGGAACUUUACUCGCAGAAAGAGCUG
CGUUCCAGCAACGUUUUUGACCUCGAAGAGAUCAUGCGCGAAUUCAACUCGUACAAGCAGUG
AUAAUAGGCUGGAGCCUCGGUGGCCAUGCUUCUUGCCCCUUGGGCCUCCCCCCAGCCCCUCC
UCCCCUUCCUGCACCCGUACCCCCGUGGUCUUUGAAUAAAGUCUGAGUGGGCGGC
hCMV TrgBFLAG, hCMV glycoproteinB ectodomain-FLAG
(SEQ ID NO: 11)
TCAAGCTTTTGGACCCTCGTACAGAAGCTAATACGACTCACTATAGGGAAATAAGAGAGAAA
AGAAGAGTAAGAAGAAATATAAGAGCCACCATGGAATCCAGGATCTGGTGCCTGGTAGTCTG
CGTTAACTTGTGTATCGTCTGTCTGGGTGCTGCGGTTTCCTCATCTTCTACTCGTGGAACTT
CTGCTACTCACAGTCACCATTCCTCTCATACGACGTCTGCTGCTCACTCTCGATCCGGTTCA
GTCTCTCAACGCGTAACTTCTTCCCAAACGGTCAGCCATGGTGTTAACGAGACCATCTACAA
CACTACCCTCAAGTACGGAGATGTGGTGGGGGTCAATACCACCAAGTACCCCTATCGCGTGT
GTTCTATGGCCCAGGGTACGGATCTTATTCGCTTTGAACGTAATATCGTCTGCACCTCGATG
AAGCCCATCAATGAAGACCTGGACGAGGGCATCATGGTGGTCTACAAACGCAACATCGTCGC
GCACACCTTTAAGGTACGAGTCTACCAGAAGGTTTTGACGTTTCGTCGTAGCTACGCTTACA
TCCACACCACTTATCTGCTGGGCAGCAACACGGAATACGTGGCGCCTCCTATGTGGGAGATT
CATCATATCAACAGCCACAGTCAGTGCTACAGTTCCTACAGCCGCGTTATAGCAGGCACGGT
TTTCGTGGCTTATCATAGGGACAGCTATGAAAACAAAACCATGCAATTAATGCCCGACGATT
ATTCCAACACCCACAGTACCCGTTACGTGACGGTCAAGGATCAATGGCACAGCCGCGGCAGC
ACCTGGCTCTATCGTGAGACCTGTAATCTGAATTGTATGGTGACCATCACTACTGCGCGCTC
CAAATATCCTTATCATTTTTTCGCCACTTCCACGGGTGACGTGGTTGACATTTCTCCTTTCT
ACAACGGAACCAATCGCAATGCCAGCTACTTTGGAGAAAACGCCGACAAGTTTTTCATTTTT
CCGAACTACACTATCGTCTCCGACTTTGGAAGACCGAATTCTGCGTTAGAGACCCACAGGTT
GGTGGCTTTTCTTGAACGTGCGGACTCGGTGATCTCCTGGGATATACAGGACGAAAAGAATG
TCACTTGTCAACTCACTTTCTGGGAAGCCTCGGAACGCACCATTCGTTCCGAAGCCGAGGAC
TCGTATCACTTTTCTTCTGCCAAAATGACCGCCACTTTCTTATCTAAGAAGCAAGAGGTGAA
CATGTCCGACTCTGCGCTGGACTGCGTACGTGATGAGGCTATAAATAAGTTACAGCAGATTT
TCAATACTTCATACAATCAAACATATGAAAAATATGGAAACGTGTCCGTCTTTGAAACCACT
GGTGGTTTGGTAGTGTTCTGGCAAGGTATCAAGCAAAAATCTCTGGTGGAACTCGAACGTTT
GGCCAACCGCTCCAGTCTGAATCTTACTCATAATAGAACCAAAAGAAGTACAGATGGCAACA
ATGCAACTCATTTATCCAACATGGAATCGGTGCACAATCTGGTCTACGCCCAGCTGCAGTTC
ACCTATGACACGTTGCGCGGTTACATCAACCGGGCGCTGGCGCAAATCGCAGAAGCCTGGTG
TGTGGATCAACGGCGCACCCTAGAGGTCTTCAAGGAACTCAGCAAGATCAACCCGTCAGCCA
TTCTCTCGGCCATTTACAACAAACCGATTGCCGCGCGTTTCATGGGTGATGTCTTGGGCCTG
GCCAGCTGCGTGACCATCAACCAAACCAGCGTCAAGGTGCTGCGTGATATGAACGTGAAGGA
GTCGCCAGGACGCTGCTACTCACGACCCGTGGTCATCTTTAATTTCGCCAACAGCTCGTACG
TGCAGTACGGTCAACTGGGCGAGGACAACGAAATCCTGTTGGGCAACCACCGCACTGAGGAA
TGTCAGCTTCCCAGCCTCAAGATCTTCATCGCCGGGAACTCGGCCTACGAGTACGTGGACTA
CCTCTTCAAACGCATGATTGACCTCAGCAGTATCTCCACCGTCGACAGCATGATCGCCCTGG
ATATCGACCCGCTGGAAAATACCGACTTCAGGGTACTGGAACTTTACTCGCAGAAAGAGCTG
CGTTCCAGCAACGTTTTTGACCTCGAAGAGATCATGCGCGAATTCAACTCGTACAAGCAGGA
TTACAAGGACGATGACGATAAGTGATAATAGGCTGGAGCCTCGGTGGCCATGCTTCTTGCCC
CTTGGGCCTCCCCCCAGCCCCTCCTCCCCTTCCTGCACCCGTACCCCCGTGGTCTTTGAATA
AAGTCTGAGTGGGCGGC
hCMV TrgBFLAG, hCMV glycoproteinB Ectodomain-FLAG mRNA
(SEQ ID NO: 168)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGGAAUCCAGGAUCUGGUGCCUGGUAGUCUG
CGUUAACUUGUGUAUCGUCUGUCUGGGUGCUGCGGUUUCCUCAUCUUCUACUCGUGGAACUU
CUGCUACUCACAGUCACCAUUCCUCUCAUACGACGUCUGCUGCUCACUCUCGAUCCGGUUCA
GUCUCUCAACGCGUAACUUCUUCCCAAACGGUCAGCCAUGGUGUUAACGAGACCAUCUACAA
CACUACCCUCAAGUACGGAGAUGUGGUGGGGGUCAAUACCACCAAGUACCCCUAUCGCGUGU
GUUCUAUGGCCCAGGGUACGGAUCUUAUUCGCUUUGAACGUAAUAUCGUCUGCACCUCGAUG
AAGCCCAUCAAUGAAGACCUGGACGAGGGCAUCAUGGUGGUCUACAAACGCAACAUCGUCGC
GCACACCUUUAAGGUACGAGUCUACCAGAAGGUUUUGACGUUUCGUCGUAGCUACGCUUACA
UCCACACCACUUAUCUGCUGGGCAGCAACACGGAAUACGUGGCGCCUCCUAUGUGGGAGAUU
CAUCAUAUCAACAGCCACAGUCAGUGCUACAGUUCCUACAGCCGCGUUAUAGCAGGCACGGU
UUUCGUGGCUUAUCAUAGGGACAGCUAUGAAAACAAAACCAUGCAAUUAAUGCCCGACGAUU
AUUCCAACACCCACAGUACCCGUUACGUGACGGUCAAGGAUCAAUGGCACAGCCGCGGCAGC
ACCUGGCUCUAUCGUGAGACCUGUAAUCUGAAUUGUAUGGUGACCAUCACUACUGCGCGCUC
CAAAUAUCCUUAUCAUUUUUUCGCCACUUCCACGGGUGACGUGGUUGACAUUUCUCCUUUCU
ACAACGGAACCAAUCGCAAUGCCAGCUACUUUGGAGAAAACGCCGACAAGUUUUUCAUUUUU
CCGAACUACACUAUCGUCUCCGACUUUGGAAGACCGAAUUCUGCGUUAGAGACCCACAGGUU
GGUGGCUUUUCUUGAACGUGCGGACUCGGUGAUCUCCUGGGAUAUACAGGACGAAAAGAAUG
UCACUUGUCAACUCACUUUCUGGGAAGCCUCGGAACGCACCAUUCGUUCCGAAGCCGAGGAC
UCGUAUCACUUUUCUUCUGCCAAAAUGACCGCCACUUUCUUAUCUAAGAAGCAAGAGGUGAA
CAUGUCCGACUCUGCGCUGGACUGCGUACGUGAUGAGGCUAUAAAUAAGUUACAGCAGAUUU
UCAAUACUUCAUACAAUCAAACAUAUGAAAAAUAUGGAAACGUGUCCGUCUUUGAAACCACU
GGUGGUUUGGUAGUGUUCUGGCAAGGUAUCAAGCAAAAAUCUCUGGUGGAACUCGAACGUUU
GGCCAACCGCUCCAGUCUGAAUCUUACUCAUAAUAGAACCAAAAGAAGUACAGAUGGCAACA
AUGCAACUCAUUUAUCCAACAUGGAAUCGGUGCACAAUCUGGUCUACGCCCAGCUGCAGUUC
ACCUAUGACACGUUGCGCGGUUACAUCAACCGGGCGCUGGCGCAAAUCGCAGAAGCCUGGUG
UGUGGAUCAACGGCGCACCCUAGAGGUCUUCAAGGAACUCAGCAAGAUCAACCCGUCAGCCA
UUCUCUCGGCCAUUUACAACAAACCGAUUGCCGCGCGUUUCAUGGGUGAUGUCUUGGGCCUG
GCCAGCUGCGUGACCAUCAACCAAACCAGCGUCAAGGUGCUGCGUGAUAUGAACGUGAAGGA
GUCGCCAGGACGCUGCUACUCACGACCCGUGGUCAUCUUUAAUUUCGCCAACAGCUCGUACG
UGCAGUACGGUCAACUGGGCGAGGACAACGAAAUCCUGUUGGGCAACCACCGCACUGAGGAA
UGUCAGCUUCCCAGCCUCAAGAUCUUCAUCGCCGGGAACUCGGCCUACGAGUACGUGGACUA
CCUCUUCAAACGCAUGAUUGACCUCAGCAGUAUCUCCACCGUCGACAGCAUGAUCGCCCUGG
AUAUCGACCCGCUGGAAAAUACCGACUUCAGGGUACUGGAACUUUACUCGCAGAAAGAGCUG
CGUUCCAGCAACGUUUUUGACCUCGAAGAGAUCAUGCGCGAAUUCAACUCGUACAAGCAGGA
UUACAAGGACGAUGACGAUAAGUGAUAAUAGGCUGGAGCCUCGGUGGCCAUGCUUCUUGCCC
CUUGGGCCUCCCCCCAGCCCCUCCUCCCCUUCCUGCACCCGUACCCCCGUGGUCUUUGAAUA
AAGUCUGAGUGGGCGGC
hCMV-TrgB6XHis, hCMV glycoprotein ectodomain-6XHis tag
(SEQ ID NO: 12)
TCAAGCTTTTGGACCCTCGTACAGAAGCTAATACGACTCACTATAGGGAAATAAGAGAGAAA
AGAAGAGTAAGAAGAAATATAAGAGCCACCATGGAATCCAGGATCTGGTGCCTGGTAGTCTG
CGTTAACTTGTGTATCGTCTGTCTGGGTGCTGCGGTTTCCTCATCTTCTACTCGTGGAACTT
CTGCTACTCACAGTCACCATTCCTCTCATACGACGTCTGCTGCTCACTCTCGATCCGGTTCA
GTCTCTCAACGCGTAACTTCTTCCCAAACGGTCAGCCATGGTGTTAACGAGACCATCTACAA
CACTACCCTCAAGTACGGAGATGTGGTGGGGGTCAATACCACCAAGTACCCCTATCGCGTGT
GTTCTATGGCCCAGGGTACGGATCTTATTCGCTTTGAACGTAATATCGTCTGCACCTCGATG
AAGCCCATCAATGAAGACCTGGACGAGGGCATCATGGTGGTCTACAAACGCAACATCGTCGC
GCACACCTTTAAGGTACGAGTCTACCAGAAGGTTTTGACGTTTCGTCGTAGCTACGCTTACA
TCCACACCACTTATCTGCTGGGCAGCAACACGGAATACGTGGCGCCTCCTATGTGGGAGATT
CATCATATCAACAGCCACAGTCAGTGCTACAGTTCCTACAGCCGCGTTATAGCAGGCACGGT
TTTCGTGGCTTATCATAGGGACAGCTATGAAAACAAAACCATGCAATTAATGCCCGACGATT
ATTCCAACACCCACAGTACCCGTTACGTGACGGTCAAGGATCAATGGCACAGCCGCGGCAGC
ACCTGGCTCTATCGTGAGACCTGTAATCTGAATTGTATGGTGACCATCACTACTGCGCGCTC
CAAATATCCTTATCATTTTTTCGCCACTTCCACGGGTGACGTGGTTGACATTTCTCCTTTCT
ACAACGGAACCAATCGCAATGCCAGCTACTTTGGAGAAAACGCCGACAAGTTTTTCATTTTT
CCGAACTACACTATCGTCTCCGACTTTGGAAGACCGAATTCTGCGTTAGAGACCCACAGGTT
GGTGGCTTTTCTTGAACGTGCGGACTCGGTGATCTCCTGGGATATACAGGACGAAAAGAATG
TCACTTGTCAACTCACTTTCTGGGAAGCCTCGGAACGCACCATTCGTTCCGAAGCCGAGGAC
TCGTATCACTTTTCTTCTGCCAAAATGACCGCCACTTTCTTATCTAAGAAGCAAGAGGTGAA
CATGTCCGACTCTGCGCTGGACTGCGTACGTGATGAGGCTATAAATAAGTTACAGCAGATTT
TCAATACTTCATACAATCAAACATATGAAAAATATGGAAACGTGTCCGTCTTTGAAACCACT
GGTGGTTTGGTAGTGTTCTGGCAAGGTATCAAGCAAAAATCTCTGGTGGAACTCGAACGTTT
GGCCAACCGCTCCAGTCTGAATCTTACTCATAATAGAACCAAAAGAAGTACAGATGGCAACA
ATGCAACTCATTTATCCAACATGGAATCGGTGCACAATCTGGTCTACGCCCAGCTGCAGTTC
ACCTATGACACGTTGCGCGGTTACATCAACCGGGCGCTGGCGCAAATCGCAGAAGCCTGGTG
TGTGGATCAACGGCGCACCCTAGAGGTCTTCAAGGAACTCAGCAAGATCAACCCGTCAGCCA
TTCTCTCGGCCATTTACAACAAACCGATTGCCGCGCGTTTCATGGGTGATGTCTTGGGCCTG
GCCAGCTGCGTGACCATCAACCAAACCAGCGTCAAGGTGCTGCGTGATATGAACGTGAAGGA
GTCGCCAGGACGCTGCTACTCACGACCCGTGGTCATCTTTAATTTCGCCAACAGCTCGTACG
TGCAGTACGGTCAACTGGGCGAGGACAACGAAATCCTGTTGGGCAACCACCGCACTGAGGAA
TGTCAGCTTCCCAGCCTCAAGATCTTCATCGCCGGGAACTCGGCCTACGAGTACGTGGACTA
CCTCTTCAAACGCATGATTGACCTCAGCAGTATCTCCACCGTCGACAGCATGATCGCCCTGG
ATATCGACCCGCTGGAAAATACCGACTTCAGGGTACTGGAACTTTACTCGCAGAAAGAGCTG
CGTTCCAGCAACGTTTTTGACCTCGAAGAGATCATGCGCGAATTCAACTCGTACAAGCAGCA
CCATCACCACCATCACTGATAATAGGCTGGAGCCTCGGTGGCCATGCTTCTTGCCCCTTGGG
CCTCCCCCCAGCCCCTCCTCCCCTTCCTGCACCCGTACCCCCGTGGTCTTTGAATAAAGTCT
GAGTGGGCGGC
hCMV-TrgB6XHis, hCMV glycoprotein ectodomain-6XHis tag mRNA
(SEQ ID NO: 169)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGGAAUCCAGGAUCUGGUGCCUGGUAGUCUG
CGUUAACUUGUGUAUCGUCUGUCUGGGUGCUGCGGUUUCCUCAUCUUCUACUCGUGGAACUU
CUGCUACUCACAGUCACCAUUCCUCUCAUACGACGUCUGCUGCUCACUCUCGAUCCGGUUCA
GUCUCUCAACGCGUAACUUCUUCCCAAACGGUCAGCCAUGGUGUUAACGAGACCAUCUACAA
CACUACCCUCAAGUACGGAGAUGUGGUGGGGGUCAAUACCACCAAGUACCCCUAUCGCGUGU
GUUCUAUGGCCCAGGGUACGGAUCUUAUUCGCUUUGAACGUAAUAUCGUCUGCACCUCGAUG
AAGCCCAUCAAUGAAGACCUGGACGAGGGCAUCAUGGUGGUCUACAAACGCAACAUCGUCGC
GCACACCUUUAAGGUACGAGUCUACCAGAAGGUUUUGACGUUUCGUCGUAGCUACGCUUACA
UCCACACCACUUAUCUGCUGGGCAGCAACACGGAAUACGUGGCGCCUCCUAUGUGGGAGAUU
CAUCAUAUCAACAGCCACAGUCAGUGCUACAGUUCCUACAGCCGCGUUAUAGCAGGCACGGU
UUUCGUGGCUUAUCAUAGGGACAGCUAUGAAAACAAAACCAUGCAAUUAAUGCCCGACGAUU
AUUCCAACACCCACAGUACCCGUUACGUGACGGUCAAGGAUCAAUGGCACAGCCGCGGCAGC
ACCUGGCUCUAUCGUGAGACCUGUAAUCUGAAUUGUAUGGUGACCAUCACUACUGCGCGCUC
CAAAUAUCCUUAUCAUUUUUUCGCCACUUCCACGGGUGACGUGGUUGACAUUUCUCCUUUCU
ACAACGGAACCAAUCGCAAUGCCAGCUACUUUGGAGAAAACGCCGACAAGUUUUUCAUUUUU
CCGAACUACACUAUCGUCUCCGACUUUGGAAGACCGAAUUCUGCGUUAGAGACCCACAGGUU
GGUGGCUUUUCUUGAACGUGCGGACUCGGUGAUCUCCUGGGAUAUACAGGACGAAAAGAAUG
UCACUUGUCAACUCACUUUCUGGGAAGCCUCGGAACGCACCAUUCGUUCCGAAGCCGAGGAC
UCGUAUCACUUUUCUUCUGCCAAAAUGACCGCCACUUUCUUAUCUAAGAAGCAAGAGGUGAA
CAUGUCCGACUCUGCGCUGGACUGCGUACGUGAUGAGGCUAUAAAUAAGUUACAGCAGAUUU
UCAAUACUUCAUACAAUCAAACAUAUGAAAAAUAUGGAAACGUGUCCGUCUUUGAAACCACU
GGUGGUUUGGUAGUGUUCUGGCAAGGUAUCAAGCAAAAAUCUCUGGUGGAACUCGAACGUUU
GGCCAACCGCUCCAGUCUGAAUCUUACUCAUAAUAGAACCAAAAGAAGUACAGAUGGCAACA
AUGCAACUCAUUUAUCCAACAUGGAAUCGGUGCACAAUCUGGUCUACGCCCAGCUGCAGUUC
ACCUAUGACACGUUGCGCGGUUACAUCAACCGGGCGCUGGCGCAAAUCGCAGAAGCCUGGUG
UGUGGAUCAACGGCGCACCCUAGAGGUCUUCAAGGAACUCAGCAAGAUCAACCCGUCAGCCA
UUCUCUCGGCCAUUUACAACAAACCGAUUGCCGCGCGUUUCAUGGGUGAUGUCUUGGGCCUG
GCCAGCUGCGUGACCAUCAACCAAACCAGCGUCAAGGUGCUGCGUGAUAUGAACGUGAAGGA
GUCGCCAGGACGCUGCUACUCACGACCCGUGGUCAUCUUUAAUUUCGCCAACAGCUCGUACG
UGCAGUACGGUCAACUGGGCGAGGACAACGAAAUCCUGUUGGGCAACCACCGCACUGAGGAA
UGUCAGCUUCCCAGCCUCAAGAUCUUCAUCGCCGGGAACUCGGCCUACGAGUACGUGGACUA
CCUCUUCAAACGCAUGAUUGACCUCAGCAGUAUCUCCACCGUCGACAGCAUGAUCGCCCUGG
AUAUCGACCCGCUGGAAAAUACCGACUUCAGGGUACUGGAACUUUACUCGCAGAAAGAGCUG
CGUUCCAGCAACGUUUUUGACCUCGAAGAGAUCAUGCGCGAAUUCAACUCGUACAAGCAGCA
CCAUCACCACCAUCACUGAUAAUAGGCUGGAGCCUCGGUGGCCAUGCUUCUUGCCCCUUGGG
CCUCCCCCCAGCCCCUCCUCCCCUUCCUGCACCCGUACCCCCGUGGUCUUUGAAUAAAGUCU
GAGUGGGCGGC

### Example 14: hCMV Vaccine—hCMV UL Sequences

A hCMV vaccine may comprise, for example, at least one RNA polynucleotide encoded by at least one of the following sequences or by at least one fragment or epitope of the following sequences. In some embodiments, a hCMV vaccine may comprise at least one RNA polynucleotide comprising at least one of the mRNA sequences listed below or at least one fragment of the mRNA sequences listed below.

hCMV UL128
(SEQ ID NO: 13)
TCAAGCTTTTGGACCCTCGTACAGAAGCTAATACGACTCACTATAGGGAAATAAGAGAGAAA
AGAAGAGTAAGAAGAAATATAAGAGCCACCATGAGTCCCAAAGATCTGACGCCGTTCTTGAC
GGCGTTGTGGCTGCTATTGGGTCACAGCCGCGTGCCGCGGGTGCGCGCAGAAGAATGTTGCG
AATTCATAAACGTCAACCACCCGCCGGAACGCTGTTACGATTTCAAAATGTGCAATCGCTTC
ACCGTCGCGCTGCGGTGTCCGGACGGCGAAGTCTGCTACAGTCCCGAGAAAACGGCTGAGAT
TCGCGGGATCGTCACCACCATGACCCATTCATTGACACGCCAGGTCGTACACAACAAACTGA
CGAGCTGCAACTACAATCCGTTATACCTCGAAGCTGACGGGCGAATACGCTGCGGCAAAGTA
AACGACAAGGCGCAGTACCTGCTGGGCGCCGCTGGCAGCGTTCCCTATCGATGGATCAATCT
GGAATACGACAAGATAACCCGGATCGTGGGCCTGGATCAGTACCTGGAGAGCGTTAAGAAAC
ACAAACGGCTGGATGTGTGCCGCGCTAAAATGGGCTATATGCTGCAGTGATAATAGGCTGGA
GCCTCGGTGGCCATGCTTCTTGCCCCTTGGGCCTCCCCCCAGCCCCTCCTCCCCTTCCTGCA
CCCGTACCCCCGTGGTCTTTGAATAAAGTCTGAGTGGGCGGC
hCMV UL128 mRNA
(SEQ ID NO: 170)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGAGUCCCAAAGAUCUGACGCCGUUCUUGAC
GGCGUUGUGGCUGCUAUUGGGUCACAGCCGCGUGCCGCGGGUGCGCGCAGAAGAAUGUUGCG
AAUUCAUAAACGUCAACCACCCGCCGGAACGCUGUUACGAUUUCAAAAUGUGCAAUCGCUUC
ACCGUCGCGCUGCGGUGUCCGGACGGCGAAGUCUGCUACAGUCCCGAGAAAACGGCUGAGAU
UCGCGGGAUCGUCACCACCAUGACCCAUUCAUUGACACGCCAGGUCGUACACAACAAACUGA
CGAGCUGCAACUACAAUCCGUUAUACCUCGAAGCUGACGGGCGAAUACGCUGCGGCAAAGUA
AACGACAAGGCGCAGUACCUGCUGGGCGCCGCUGGCAGCGUUCCCUAUCGAUGGAUCAAUCU
GGAAUACGACAAGAUAACCCGGAUCGUGGGCCUGGAUCAGUACCUGGAGAGCGUUAAGAAAC
ACAAACGGCUGGAUGUGUGCCGCGCUAAAAUGGGCUAUAUGCUGCAGUGAUAAUAGGCUGGA
GCCUCGGUGGCCAUGCUUCUUGCCCCUUGGGCCUCCCCCCAGCCCCUCCUCCCCUUCCUGCA
CCCGUACCCCCGUGGUCUUUGAAUAAAGUCUGAGUGGGCGGC
hCMV-128FLAG, UL128-FLAGtag
(SEQ ID NO: 14)
TCAAGCTTTTGGACCCTCGTACAGAAGCTAATACGACTCACTATAGGGAAATAAGAGAGAAA
AGAAGAGTAAGAAGAAATATAAGAGCCACCATGAGTCCCAAAGATCTGACGCCGTTCTTGAC
GGCGTTGTGGCTGCTATTGGGTCACAGCCGCGTGCCGCGGGTGCGCGCAGAAGAATGTTGCG
AATTCATAAACGTCAACCACCCGCCGGAACGCTGTTACGATTTCAAAATGTGCAATCGCTTC
ACCGTCGCGCTGCGGTGTCCGGACGGCGAAGTCTGCTACAGTCCCGAGAAAACGGCTGAGAT
TCGCGGGATCGTCACCACCATGACCCATTCATTGACACGCCAGGTCGTACACAACAAACTGA
CGAGCTGCAACTACAATCCGTTATACCTCGAAGCTGACGGGCGAATACGCTGCGGCAAAGTA
AACGACAAGGCGCAGTACCTGCTGGGCGCCGCTGGCAGCGTTCCCTATCGATGGATCAATCT
GGAATACGACAAGATAACCCGGATCGTGGGCCTGGATCAGTACCTGGAGAGCGTTAAGAAAC
ACAAACGGCTGGATGTGTGCCGCGCTAAAATGGGCTATATGCTGCAGGATTACAAGGACGAT
GACGATAAGTGATAATAGGCTGGAGCCTCGGTGGCCATGCTTCTTGCCCCTTGGGCCTCCCC
CCAGCCCCTCCTCCCCTTCCTGCACCCGTACCCCCGTGGTCTTTGAATAAAGTCTGAGTGGG
CGGC
hCMV-128FLAG, UL128-FLAG tag mRNA
(SEQ ID NO: 171)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGAGUCCCAAAGAUCUGACGCCGUUCUUGAC
GGCGUUGUGGCUGCUAUUGGGUCACAGCCGCGUGCCGCGGGUGCGCGCAGAAGAAUGUUGCG
AAUUCAUAAACGUCAACCACCCGCCGGAACGCUGUUACGAUUUCAAAAUGUGCAAUCGCUUC
ACCGUCGCGCUGCGGUGUCCGGACGGCGAAGUCUGCUACAGUCCCGAGAAAACGGCUGAGAU
UCGCGGGAUCGUCACCACCAUGACCCAUUCAUUGACACGCCAGGUCGUACACAACAAACUGA
CGAGCUGCAACUACAAUCCGUUAUACCUCGAAGCUGACGGGCGAAUACGCUGCGGCAAAGUA
AACGACAAGGCGCAGUACCUGCUGGGCGCCGCUGGCAGCGUUCCCUAUCGAUGGAUCAAUCU
GGAAUACGACAAGAUAACCCGGAUCGUGGGCCUGGAUCAGUACCUGGAGAGCGUUAAGAAAC
ACAAACGGCUGGAUGUGUGCCGCGCUAAAAUGGGCUAUAUGCUGCAGGAUUACAAGGACGAU
GACGAUAAGUGAUAAUAGGCUGGAGCCUCGGUGGCCAUGCUUUUUGCCCCUUGGGCCUCCCC
CCAGCCCCUCCUCCCCUUCCUGCACCCGUACCCCCGUGGUCUUUGAAUAAAGUCUGAGUGGG
CGGC
hCMV-UL130
(SEQ ID NO: 15)
TCAAGCTTTTGGACCCTCGTACAGAAGCTAATACGACTCACTATAGGGAAATAAGAGAGAAA
AGAAGAGTAAGAAGAAATATAAGAGCCACCATGCTGCGGCTTCTGCTTCGTCACCACTTTCA
CTGCCTGCTTCTGTGCGCGGTTTGGGCAACGCCCTGTCTGGCGTCTCCGTGGTCGACGCTAA
CAGCAAACCAGAATCCGTCCCCGCCATGGTCTAAACTGACGTATTCCAAACCGCATGACGCG
GCGACGTTTTACTGTCCTTTTCTCTATCCCTCGCCCCCACGATCCCCCTTGCAATTCTCGGG
GTTCCAGCGGGTATCAACGGGTCCCGAGTGTCGCAACGAGACCCTGTATCTGCTGTACAACC
GGGAAGGCCAGACCTTGGTGGAGAGAAGCTCCACCTGGGTGAAAAAGGTGATCTGGTACCTG
AGCGGTCGGAACCAAACCATCCTCCAACGGATGCCCCGAACGGCTTCGAAACCGAGCGACGG
AAACGTGCAGATCAGCGTGGAAGACGCCAAGATTTTTGGAGCGCACATGGTGCCCAAGCAGA
CCAAGCTGCTACGCTTCGTCGTCAACGATGGCACACGTTATCAGATGTGTGTGATGAAGCTG
GAGAGCTGGGCTCACGTCTTCCGGGACTACAGCGTGTCTTTTCAGGTGCGATTGACGTTCAC
CGAGGCCAATAACCAGACTTACACCTTCTGCACCCATCCCAATCTCATCGTTTGATAATAGG
CTGGAGCCTCGGTGGCCATGCTTCTTGCCCCTTGGGCCTCCCCCCAGCCCCTCCTCCCCTTC
CTGCACCCGTACCCCCGTGGTCTTTGAATAAAGTCTGAGTGGGCGGC
hCMV-UL130 mRNA
(SEQ ID NO: 172)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCUGCGGCUUCUGCUUCGUCACCACUUUCA
CUGCCUGCUUCUGUGCGCGGUUUGGGCAACGCCCUGUCUGGCGUCUCCGUGGUCGACGCUAA
CAGCAAACCAGAAUCCGUCCCCGCCAUGGUCUAAACUGACGUAUUCCAAACCGCAUGACGCG
GCGACGUUUUACUGUCCUUUUCUCUAUCCCUCGCCCCCACGAUCCCCCUUGCAAUUCUCGGG
GUUCCAGCGGGUAUCAACGGGUCCCGAGUGUCGCAACGAGACCCUGUAUCUGCUGUACAACC
GGGAAGGCCAGACCUUGGUGGAGAGAAGCUCCACCUGGGUGAAAAAGGUGAUCUGGUACCUG
AGCGGUCGGAACCAAACCAUCCUCCAACGGAUGCCCCGAACGGCUUCGAAACCGAGCGACGG
AAACGUGCAGAUCAGCGUGGAAGACGCCAAGAUUUUUGGAGCGCACAUGGUGCCCAAGCAGA
CCAAGCUGCUACGCUUCGUCGUCAACGAUGGCACACGUUAUCAGAUGUGUGUGAUGAAGCUG
GAGAGCUGGGCUCACGUCUUCCGGGACUACAGCGUGUCUUUUCAGGUGCGAUUGACGUUCAC
CGAGGCCAAUAACCAGACUUACACCUUCUGCACCCAUCCCAAUCUCAUCGUUUGAUAAUAGG
CUGGAGCCUCGGUGGCCAUGCUUCUUGCCCCUUGGGCCUCCCCCCAGCCCCUCCUCCCCUUC
CUGCACCCGUACCCCCGUGGUCUUUGAAUAAAGUCUGAGUGGGCGGC
hCMV-UL130FLAG, UL130-FLAG tag
(SEQ ID NO: 16)
TCAAGCTTTTGGACCCTCGTACAGAAGCTAATACGACTCACTATAGGGAAATAAGAGAGAAA
AGAAGAGTAAGAAGAAATATAAGAGCCACCATGCTGCGGCTTCTGCTTCGTCACCACTTTCA
CTGCCTGCTTCTGTGCGCGGTTTGGGCAACGCCCTGTCTGGCGTCTCCGTGGTCGACGCTAA
CAGCAAACCAGAATCCGTCCCCGCCATGGTCTAAACTGACGTATTCCAAACCGCATGACGCG
GCGACGTTTTACTGTCCTTTTCTCTATCCCTCGCCCCCACGATCCCCCTTGCAATTCTCGGG
GTTCCAGCGGGTATCAACGGGTCCCGAGTGTCGCAACGAGACCCTGTATCTGCTGTACAACC
GGGAAGGCCAGACCTTGGTGGAGAGAAGCTCCACCTGGGTGAAAAAGGTGATCTGGTACCTG
AGCGGTCGGAACCAAACCATCCTCCAACGGATGCCCCGAACGGCTTCGAAACCGAGCGACGG
AAACGTGCAGATCAGCGTGGAAGACGCCAAGATTTTTGGAGCGCACATGGTGCCCAAGCAGA
CCAAGCTGCTACGCTTCGTCGTCAACGATGGCACACGTTATCAGATGTGTGTGATGAAGCTG
GAGAGCTGGGCTCACGTCTTCCGGGACTACAGCGTGTCTTTTCAGGTGCGATTGACGTTCAC
CGAGGCCAATAACCAGACTTACACCTTCTGCACCCATCCCAATCTCATCGTTGATTACAAGG
ACGATGACGATAAGTGATGATAATAGGCTGGAGCCTCGGTGGCCATGCTTCTTGCCCCTTGG
GCCTCCCCCCAGCCCCTCCTCCCCTTCCTGCACCCGTACCCCCGTGGTCTTTGAATAAAGTC
TGAGTGGGCGGC
hCMV-UL130FLAG, UL130-FLAG tag mRNA
(SEQ ID NO: 173)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCUGCGGCUUCUGCUUCGUCACCACUUUCA
CUGCCUGCUUCUGUGCGCGGUUUGGGCAACGCCCUGUCUGGCGUCUCCGUGGUCGACGCUAA
CAGCAAACCAGAAUCCGUCCCCGCCAUGGUCUAAACUGACGUAUUCCAAACCGCAUGACGCG
GCGACGUUUUACUGUCCUUUUCUCUAUCCCUCGCCCCCACGAUCCCCCUUGCAAUUCUCGGG
GUUCCAGCGGGUAUCAACGGGUCCCGAGUGUCGCAACGAGACCCUGUAUCUGCUGUACAACC
GGGAAGGCCAGACCUUGGUGGAGAGAAGCUCCACCUGGGUGAAAAAGGUGAUCUGGUACCUG
AGCGGUCGGAACCAAACCAUCCUCCAACGGAUGCCCCGAACGGCUUCGAAACCGAGCGACGG
AAACGUGCAGAUCAGCGUGGAAGACGCCAAGAUUUUUGGAGCGCACAUGGUGCCCAAGCAGA
CCAAGCUGCUACGCUUCGUCGUCAACGAUGGCACACGUUAUCAGAUGUGUGUGAUGAAGCUG
GAGAGCUGGGCUCACGUCUUCCGGGACUACAGCGUGUCUUUUCAGGUGCGAUUGACGUUCAC
CGAGGCCAAUAACCAGACUUACACCUUCUGCACCCAUCCCAAUCUCAUCGUUGAUUACAAGG
ACGAUGACGAUAAGUGAUGAUAAUAGGCUGGAGCCUCGGUGGCCAUGCUUCUUGCCCCUUGG
GCCUCCCCCCAGCCCCUCCUCCCCUUCCUGCACCCGUACCCCCGUGGUCUUUGAAUAAAGUC
UGAGUGGGCGGC
hCMV-UL131A
(SEQ ID NO: 17)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCTGTGTCGGGTGTGGCTGTCTGTTTG
TCTGTGCGCCGTGGTGCTGGGTCAGTGCCAGCGGGAAACCGCGGAAAAAAACGATTATTACC
GAGTACCGCATTACTGGGACGCGTGCTCTCGCGCGCTGCCCGACCAAACCCGTTACAAGTAT
GTGGAACAGCTCGTGGACCTCACGTTGAACTACCACTACGATGCGAGCCACGGCTTGGACAA
CTTTGACGTGCTCAAGAGAATCAACGTGACCGAGGTGTCGTTGCTCATCAGCGACTTTAGAC
GTCAGAACCGTCGCGGCGGCACCAACAAAAGGACCACGTTCAACGCCGCCGGTTCGCTGGCG
CCACACGCCCGGAGCCTCGAGTTCAGCGTGCGGCTCTTTGCCAACTGATAATAGGCTGGAGC
CTCGGTGGCCATGCTTCTTGCCCCTTGGGCCTCCCCCCAGCCCCTCCTCCCCTTCCTGCACC
CGTACCCCCGTGGTCTTTGAATAAAGTCTGAGTGGGCGGC
hCMV-UL131A mRNA
(SEQ ID NO: 174)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCUGUGUCGGGUGUGGCUGUCUGUUUG
UCUGUGCGCCGUGGUGCUGGGUCAGUGCCAGCGGGAAACCGCGGAAAAAAACGAUUAUUACC
GAGUACCGCAUUACUGGGACGCGUGCUCUCGCGCGCUGCCCGACCAAACCCGUUACAAGUAU
GUGGAACAGCUCGUGGACCUCACGUUGAACUACCACUACGAUGCGAGCCACGGCUUGGACAA
CUUUGACGUGCUCAAGAGAAUCAACGUGACCGAGGUGUCGUUGCUCAUCAGCGACUUUAGAC
GUCAGAACCGUCGCGGCGGCACCAACAAAAGGACCACGUUCAACGCCGCCGGUUCGCUGGCG
CCACACGCCCGGAGCCUCGAGUUCAGCGUGCGGCUCUUUGCCAACUGAUAAUAGGCUGGAGC
CUCGGUGGCCAUGCUUCUUGCCCCUUGGGCCUCCCCCCAGCCCCUCCUCCCCUUCCUGCACC
CGUACCCCCGUGGUCUUUGAAUAAAGUCUGAGUGGGCGGC
hCMV-UL131AFLAG, UL131A-FLAG
(SEQ ID NO: 18)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCTGTGTCGGGTGTGGCTGTCTGTTTG
TCTGTGCGCCGTGGTGCTGGGTCAGTGCCAGCGGGAAACCGCGGAAAAAAACGATTATTACC
GAGTACCGCATTACTGGGACGCGTGCTCTCGCGCGCTGCCCGACCAAACCCGTTACAAGTAT
GTGGAACAGCTCGTGGACCTCACGTTGAACTACCACTACGATGCGAGCCACGGCTTGGACAA
CTTTGACGTGCTCAAGAGAATCAACGTGACCGAGGTGTCGTTGCTCATCAGCGACTTTAGAC
GTCAGAACCGTCGCGGCGGCACCAACAAAAGGACCACGTTCAACGCCGCCGGTTCGCTGGCG
CCACACGCCCGGAGCCTCGAGTTCAGCGTGCGGCTCTTTGCCAACGATTACAAGGACGATGA
CGATAAGTGATGATAATAGGCTGGAGCCTCGGTGGCCATGCTTCTTGCCCCTTGGGCCTCCC
CCCAGCCCCTCCTCCCCTTCCTGCACCCGTACCCCCGTGGTCTTTGAATAAAGTCTGAGTGG
GCGGC
hCMV-UL131AFLAG, UL131A-FLAG mRNA
(SEQ ID NO: 175)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCUGUGUCGGGUGUGGCUGUCUGUUUG
UCUGUGCGCCGUGGUGCUGGGUCAGUGCCAGCGGGAAACCGCGGAAAAAAACGAUUAUUACC
GAGUACCGCAUUACUGGGACGCGUGCUCUCGCGCGCUGCCCGACCAAACCCGUUACAAGUAU
GUGGAACAGCUCGUGGACCUCACGUUGAACUACCACUACGAUGCGAGCCACGGCUUGGACAA
CUUUGACGUGCUCAAGAGAAUCAACGUGACCGAGGUGUCGUUGCUCAUCAGCGACUUUAGAC
GUCAGAACCGUCGCGGCGGCACCAACAAAAGGACCACGUUCAACGCCGCCGGUUCGCUGGCG
CCACACGCCCGGAGCCUCGAGUUCAGCGUGCGGCUCUUUGCCAACGAUUACAAGGACGAUGA
CGAUAAGUGAUGAUAAUAGGCUGGAGCCUCGGUGGCCAUGCUUCUUGCCCCUUGGGCCUCCC
CCCAGCCCCUCCUCCCCUUCCUGCACCCGUACCCCCGUGGUCUUUGAAUAAAGUCUGAGUGG
GCGGC

### Example 15: hCMV Vaccine—hCMV UL Multimeric Sequences

A hCMV vaccine may comprise, for example, at least one RNA polynucleotide encoded by at least one of the following sequences or by at least one fragment or epitope of the following sequences. In some embodiments, a hCMV vaccine may comprise at least one RNA polynucleotide comprising at least one of the mRNA sequences listed below or at least one fragment of the mRNA sequences listed below.

hCMV gH penta
(SEQ ID NO: 19)
TCAAGCTTTTGGACCCTCGTACAGAAGCTAATACG
ACTCACTATAGGGACAGACGAGAGAGAAGCACGCC
AATTCTGCCTGCTTAAGCCATGCGGCCAGGCCTCC
CCTCCTACCTCATCATCCTCGCCGTCTGTCTCTTC
AGCCACCTACTTTCGTCACGATATGGCGCAGAAGC
CGTATCCGAACCGCTGGACAAAGCGTTTCACCTAC
TGCTCAACACCTACGGGAGACCCATCCGCTTCCTG
CGTGAAAATACCACCCAGTGTACCTACAACAGCAG
CCTCCGTAACAGCACGGTCGTCAGGGAAAACGCCA
TCAGTTTGAACTTTTTCCAAAGCTATAATCAATAC
TATGTATTCCATATGCCTCGATGTCTTTTTGCGGG
TCCTCTGGCGGAGCAGTTTCTGAACCAGGTAGATC
TGACCGAAACCCTGGAAAGATACCAACAGAGACTT
AACACTTACGCGCTGGTATCCAAAGACCTGGCCAG
CTACCGATCTTTTTCGCAGCAGCTAAAGGCACAAG
ACAGCCTAGGTGAACAGCCCACCACTGTGCCACCG
CCCATTGACCTGTCAATACCTCACGTTTGGATGCC
ACCGCAAACCACTCCACACGGCTGGACAGAATCAC
ATACCACCTCAGGACTACACCGACCACACTTTAAC
CAGACCTGTATCCTCTTTGATGGACACGATCTACT
ATTCAGCACCGTCACACCTTGTTTGCACCAAGGCT
TTTACCTCATCGACGAACTACGTTACGTTAAAATA
ACACTGACCGAGGACTTCTTCGTACTTACGCTGTC
CATAGACGACGACACACCCATGCTGCTTATCTTCG
GCCATCTTCCACGCGTACTTTTCAAAGCGCCCTAT
CAACGCGACAACTTTATACTACGACAAACTGAAAA
ACACGAGCTCCTGGTGCTAGTTAAGAAAGATCAAC
TGAACCGTCACTCTTATCTCAAAGACCCGGACTTT
CTTGACGCCGCACTTGACTTCAACTACCTAGACCT
CAGCGCACTACTACGTAACAGCTTTCACCGTTACG
CCGTGGATGTACTCAAGAGCGGTCGATGTCAGATG
CTGGACCGCCGCACGGTAGAAATGGCCTTCGCCTA
CGCATTAGCACTGTTCGCAGCAGCCCGACAAGAAG
AGGCCGGCGCCCAAGTCTCCGTCCCACGGGCCCTA
GACCGCCAGGCCGCACTCTTACAAATACAAGAATT
TATGATCACCTGCCTCTCACAAACACCACCACGCA
CCACGTTGCTGCTGTATCCCACGGCCGTGGACCTG
GCCAAACGAGCCCTTTGGACACCGAATCAGATCAC
CGACATCACCAGCCTCGTACGCCTGGTCTACATAC
TCTCTAAACAGAATCAGCAACATCTCATCCCCCAA
TGGGCACTACGACAGATCGCCGACTTTGCCCTAAA
ACTACACAAAACGCACCTGGCCTCTTTTCTTTCAG
CCTTCGCACGCCAAGAACTCTACCTCATGGGCAGC
CTCGTCCACTCCATGCTGGTACATACGACGGAGAG
ACGCGAAATCTTCATCGTAGAAACGGGCCTCTGTT
CATTGGCCGAGCTATCACACTTTACGCAGTTGTTA
GCTCATCCACACCACGAATACCTCAGCGACCTGTA
CACACCCTGTTCCAGTAGCGGGCGACGCGATCACT
CGCTCGAACGCCTCACGCGTCTCTTCCCCGATGCC
ACCGTCCCCGCTACCGTTCCCGCCGCCCTCTCCAT
CCTATCTACCATGCAACCAAGCACGCTGGAAACCT
TCCCCCACCTGTTTTGCTTGCCGCTCGGCGAATCC
TTCTCCGCGCTGACCGTCTCCGAACACGTCAGTTA
TATCGTAACAAACCAGTACCTGATCAAAGGTATCT
CCTACCCTGTCTCCACCACCGTCGTAGGCCAGAGC
CTCATCATCACCCAGACGGACAGTCAAACTAAATG
CGAACTGACGCGCAACATGCATACCACACACAGCA
TCACAGTGGCGCTCAACATTTCGCTAGAAAACTGC
GCCTTTTGCCAAAGCGCCCTGCTAGAATACGACGA
CACGCAAGGCGTCATCAACATCATGTACATGCACG
ACTCGGACGACGTCCTTTTCGCCCTGGATCCCTAC
AACGAAGTGGTGGTCTCATCTCCGCGAACTCACTA
CCTCATGCTTTTGAAAAACGGTACGGTACTAGAAG
TAACTGACGTCGTCGTGGACGCCACCGACAGTCGT
CTCCTCATGATGTCCGTCTACGCGCTATCGGCCAT
CATCGGCATCTATCTGCTCTACCGCATGCTCAAGA
CATGCTGATAATAGGCTGGAGCCTCGGTGGCCATG
CTTCTTGCCCCTTGGGCCTCCCCCCAGCCCCTCCT
CCCCTTCCTGCACCCGTACCCCCGTGGTCTTTGAA
TAAAGTCTGAGTGGGCGGC
hCMV gH penta mRNA
(SEQ ID NO: 176)
UCAAGCUUUUGGACCCUCGUACAGAAGCUAAUACG
ACUCACUAUAGGGACAGACGAGAGAGAAGCACGCC
AAUUCUGCCUGCUUAAGCCAUGCGGCCAGGCCUCC
CCUCCUACGUCAUCAUCCUCGCCGUCUGUCUCUUC
AGCCACCUACUUUCGUCACGAUAUGGCGCAGAAGC
CGUAUCCGAACCGCUGGACAAAGCGUUUCACCUAC
UGCUCAACAGCUACGGGAGACGCAUCCGCUUCCUG
CGUGAAAAUACCACCCAGUGUACCUACAACAGCAG
CCUCCGUAACAGCACGGUCGUCAGGGAAAACGCCA
UCAGUUUCAACUUUUUCCAAAGCUAUAAUCAAUAC
UAUGUAUUCCAUAUGCCUCGAUGUCUUUUUGCGGG
UCCUCUGGCGGAGCAGUUUCUGAACCAGGUAGAUC
UGACCGAAACCCUGGAAAGAUACCAACAGAGACUU
AACACUUACGCGCUGGUAUCCAAAGACCUGGCCAG
CUACCGAUCUUUUUCGCAGCAGCUAAAGGCACAAG
ACAGCCUAGGUGAACAGCCCACCACUGUGCCACCG
CCCAUUGACCUGUCAAUACCUCACGUUUGGAUGCC
ACCGCAAACCACUCCACACGGCUGGACAGAAUCAC
AUACCACCUCAGGACUACACCGACCACACUUUAAC
CAGACCUGUAUCCUCUUUGAUGGACACGAUCUACU
AUUCAGCACCGUCACACCUUGUUUGCACCAAGGCU
UUUACCUCAUCGACGAACUACGUUACGUUAAAAUA
ACACUGACCGAGGACUUCUUCGUAGUUACGGUGUC
CAUAGACGACGACACACCCAUGCUGCUUAUCUUCG
GCCAUCUUCCACGCGUACUUUUCAAAGCGCCCUAU
CAACGCGACAACUUUAUACUACGACAAACUGAAAA
ACACGAGCUCCUGGUGCUAGUUAAGAAAGAUCAAC
UGAACCGUCACUCUUAUCUCAAAGACCCGGACUUU
CUUGACGCCGCACUUGACUUCAACUACCUAGACCU
CAGCGCACUACUACGUAACAGCUUUCACCGUUACG
CCGUGGAUGUACUCAAGAGCGGUCGAUGUCAGAUG
CUGGACCGCCGCACGGUAGAAAUGGCCUUCGCCUA
CGCAUUAGCACUGUUCGCAGCAGCCCGACAAGAAG
AGGCCGGCGCCCAAGUCUCCGUCCCACGGGCCCUA
GACCGCCAGGCCGCACUCUUACAAAUACAAGAAUU
UAUGAUCACCUGCCUCUCACAAACACCACCACGCA
CCACGUUGCUGCUGUAUCCCACGGCCGUGGACCUG
GCCAAACGAGCCCUUUGGACACCGAAUCAGAUCAC
CGACAUCACCAGCCUCGUACGCCUGGUCUACAUAC
UCUCUAAACAGAAUCAGCAACAUCUCAUCCCCCAA
UGGGCACUACGACAGAUCGCCGACUUUGCCCUAAA
ACUACACAAAACGCACCUGGCCUCUUUUCUUUCAG
CCUUCGCACGCCAAGAACUCUACCUCAUGGGCAGC
CUCGUCCACUCCAUGCUGGUACAUACGACGGAGAG
ACGCGAAAUCUUCAUCGUAGAAACGGGCCUCUGUU
CAUUGGCCG
AGCUAUCACACUUUACGCAGUUGUUAGCUCAUCCA
CACCACGAAUACCUCAGCGACCUGUACACACCCUG
UUCCAGUAGCGGGCGACGCGAUCACUCGCUCGAAC
GCCUCACGCGUCUCUUCCCCGAUGCCACCGUCCCC
GCUACCGUUCCCGCCGCCCUCUCCAUCCUAUGUAC
CAUGCAACCAAGCACGCUGGAAACCUUCCCCGACC
UGUUUUGCUUGCCGCUCGGCGAAUCCUUCUCCGCG
CUGACCGUCUCCGAACACGUCAGUUAUAUCGUAAC
AAACCAGUACCUGAUCAAAGGUAUCUCCUACCCUG
UCUCCACCACCGUCGUAGGCCAGAGCCUCAUCAUC
ACGGAGACGGACAGUCAAACUAAAUGCGAACUGAC
GCGCAACAUGCAUACCACACACAGCAUCACAGUGG
CGCUCAACAUUUCGCUAGAAAACUGCGCCUUUUGC
CAAAGCGCCCUGCUAGAAUACGACGACACGCAAGG
CGUCAUCAACAUCAUGUACAUGCACGACUCGGACG
ACGUCCUUUUCGCCCUGGAUCCCUACAACGAAGUG
GUGGUCUCAUCUCCGCGAACUCACUACCUCAUGCU
UUUGAAAAACGGUACGGUACUAGAAGUAACUGACG
UCGUCGUGGACGCCACCGACAGUCGUCUCCUCAUG
AUGUCCGUCUACGCGCUAUCGGCCAUCAUCGGCAU
CUAUCUGCUCUACCGCAUGCUCAAGACAUGCUGAU
AAUAGGCUGGAGCCUCGGUGGCCAUGCUUCUUGCC
CCUUGGGCCUCCCCCCAGCCCCUCCUCCCCUUCCU
GCACCCGUACCCCCGUGGUCUUUGAAUAAAGUCUG
AGUGGGCGGC
hCMV gL penta
(SEQ ID NO: 20)
TCAAGCTTTTGGACCCTCGTACAGAAGCTAATACG
ACTCACTATAGGGCTTAAGCAGGCAGAATTGGCCC
TTAGCCTGTACCAGCCGAACCATGTGCCGCCGCCC
GGATTGCGGCTTCTCTTTCTCACCTGGACCGGTGA
TACTGCTGTGGTGTTGCCTTGTGCTGCCCATTGTT
TCCTCAGCCGCCGTCAGCGTCGCTCCTACCGCCGC
CGAGAAAGTCCCCGCGGAGTGCCCCGAACTAACGC
GCCGATGCTTGTTGGGTGAGGTGTTTGAGGGTGAC
AAGTATGAAAGTTGGCTGCGCCCGTTGGTGAATGT
TACCGGGCGCGATGGCCCGCTATCGGAACTTATGC
GTTACCGTCCCGTTAGGCCGGAGGCCGCCAACTCG
GTGCTGTTGGACGAGGCTTTCCTGGACACTCTGGC
CCTGCTGTACAACAATCCGGATCAATTGCGGGCCC
TGCTGACGCTGTTGAGCTCGGACACAGCGCCGCGC
TGGATGACGGTGATGCGCGGCTACAGCGAGTGCGG
CGATGGCTCGCCGGCCGTGTACACGTGCGTGGACG
ACCTGTGCCGCGGCTACGACCTCACGCGACTGTCA
TACGGGCGCAGCATGTTCACGGAACACGTGTTAGG
CTTCGAGCTGGTGCCACCGTCTCTCTTTAACGTGG
TGGTGGCCATACGCAACGAAGCCACGCGTACCAAC
CGCGCCGTGCGTCTGCCCGTGAGCACCGCTGCCGC
GGCCGAGGGCATCACGCTCTTTTACGGCCTGTACA
ACGCAGTGAAGGAATTCTGCCTGCGTCAGCAGCTG
GAGCCGCCGCTGCTACGCCACCTAGATAAATACTA
CGCCGGAGTGCCGCGCGAGCTGAAGCAGACGCGCG
TCAACCTGCCGGCTCACTCGCGCTATGGCCCTCAA
GCAGTGGATGCTCGCTGATAATAGGCTGGAGCCTC
GGTGGCCATGCTTCTTGCCCCTTGGGCCTCCCCCC
AGCCCCTCCTCCCCTTCCTGCACCCGTACCCCCGT
GGTCTTTGAATAAAGTCTGAGTGGGCGGC
hCMV gL penta mRNA
(SEQ ID NO: 177)
UCAAGCUUUUGGACCCUCGUACAGAAGCUAAUACG
ACUCACUAUAGGGCUUAAGCAGGCAGAAUUGGCCC
UUAGCCUGUACCAGCCGAACCAUGUGCCGCCGCCC
GGAUUGCGGCUUCUCUUUCUCACCUGGACCGGUGA
UACUGCUGUGGUGUUGCCUUCUGCUGCCCAUUGUU
UCCUGAGCGGCCGUCAGCGUCGCUCCUACCGCCGC
CGAGAAAGUCCCCGCGGAGUGCCCCGAACUAACGC
GCCGAUGCUUGUUGGGUGAGGUGUUUGAGGGUGAC
AAGUAUGAAAGUUGGCUGCGCCCGUUGGUGAAUGU
UACCGGGGGGGAUGGGCCGGUAUCGCAAGUUAUCG
GUUACGGUCCGGUUACGCCGGAGGCCGCCAACUCC
GUGCUGUUGGACGAGGCUUUCCUGGACACUCUGGC
CCUGCUGUACAACAAUCCGGAUCAAUUGCGGGCCC
UGCUGACGCUGUUGAGCUCGGACACAGCGCGGCGC
UGGAUGACGGUGAUGCGCGGCUACAGCGAGUGCGG
CGAUGGCUCGCCGGCCGUGUACACGUGCGUGGACG
ACCUGUGCCGCGGCUACGACCUCACGCGACUGUCA
UACGGGCGCAGCAUCUUCACGGAACAGGUGUUAGG
CUUCGAGCUGGUGCCACCGUCUCUCUUUAACGUGG
UGGUGGCCAUAGGCAACGAAGCCACGCGUACCAAC
CGCGCCGUGCGUCUGCCCGUGAGCACCGCUGCCGC
GCCCGAGGGCAUCACGCUCUUUUACGGCCUGUACA
ACGCAGUGAAGGAAUUGUGCCUGGGUGACCAGCUG
GACGCGCCGCUGCUACGCCACCUAGAUAAAUAGUA
CGCCGGACUGCCGCGCGAGCUGAAGCAGACGCGCG
UCAACCUGCCGGCUCACUCGCGCUAUGGCCCUCAA
GCAGUGGAUGCUCGCUGAUAAUAGGCUGGAGCCUC
GGUGGCCAUGCUUCUUGCCCCUUGGGCCUCCCCCC
AGCCCCUCCUCCCCUUCCUGCACCCGUACCCCCGU
GGUCUUUGAAUAAAGUCUGAGUGGGCGGC
hCMV gL dimer
(SEQ ID NO: 21)
TCAAGCTTTTGGACCCTCGTACAGAAGCTAATACG
ACTCACTATAGGGTGGCTCTTATATTTCTTCTTAC
TCTTCTTTTCTCTCTTATTTCCATGTGCCGCCGCC
CGGATTGCGGCTTCTCTTTCTCACCTGGACCGGTG
ATACTGCTGTGGTGTTGCCTTCTGCTGCCCATTGT
TTCCTCAGCCGCCGTCAGCGTCGCTCCTACCGCCG
CCGAGAAAGTCCCCGCGGAGTGCCCCGAACTAACG
CGCCGATGCTTGTTGGGTGAGGTGTTTGAGGGTGA
CAAGTATGAAAGTTGGCTGCGCCCGTTGGTGAATG
TTACCGGGCGCGATGGCCCGCTATCGCAACTTATC
CGTTACCGTCCCGTTACGCCGGAGGCCGCCAACTC
CGTGCTGTTGGACGAGGCTTTCCTGGACACTCTGG
CCCTGCTGTACAACAATCCGGATCAATTGCGGGCC
CTGCTGACGCTGTTGAGCTCGGACACAGCGCCGCG
CTGGATGACGGTGATGCGCGGCTACAGCGAGTGCG
GCGATGGCTCGCCGGCCGTGTACACGTGCGTGGAC
GACCTGTGCGGCGGCTACGACCTCACGCGACTGTC
ATACGGGCGCAGCATCTTCACGGAACAGGTGTTAG
GCTTCGAGCTGGTGCCACCGTCTCTCTTTAACGTG
GTGGTGGCCATACGCAACGAAGCCACGCGTACCAA
CCGCGCCGTGCGTCTGCCCGTGAGCACCGCTGCCG
CGCCCGAGGGCATCACGCTCTTTTACGGCCTGTAC
AACGCAGTGAAGGAATTCTGCCTGCGTCACCAGCT
GGACCCGCCGCTGCTACGCCACCTAGATAAATACT
ACGCCGGACTGCCGCCCGAGCTGAAGCAGACGCGC
GTCAACCTGCCGGCTCACTCGCGCTATGGCCCTCA
AGCAGTGGATGCTCGCTGATAATAGGCTGGAGCCT
CGGTGGCCATGCTTCTTGCCCCTTGGGCCTCCCCC
CAGCCCCTCCTCCCCTTCCTGCACCCGTACCCCCG
TGGTCTTTGAATAAAGTCTGAGTGGGCGGC
hCMV gL dimer mRNA
(SEQ ID NO: 178)
UCAAGCUUUUGGACCCUCGUACAGAAGCUAAUACG
ACUCACUAUAGGGUGGCUCUUAUAUUUCUUCUUAC
UCUUCUUUUCUCUCUUAUUUCCAUGUGCCGCCGCC
CGGAUUGCGGCUUCUCUUUCUCACCUGGACCGGUG
AUACUGCUGUGGUGUUGCCUUCUGCUGCCCAUUGU
UUCCUCAGCCGCCGUCAGCGUCGCUCCUACCGCCG
CCGAGAAAGUCCCCGCGGAGUGCCCCGAACUAACG
CGCCGAUGCUUGUUGGGUGAGGUGUUUGAGGGUGA
CAAGUAUGAAAGUUGGCUGCGCCCGUUGGUGAAUG
UUACCGGGCGCGAUGGCCCGCUAUCGCAACUUAUC
CGUUACCGUCCCGUUACGCCGGAGGCCGCCAACUC
CGUGCUGUUGGACGAGGCUUUCCUGGACACUCUGG
CCCUGCUGUACAACAAUCCGGAUCAAUUGCGGGCC
CUGCUGAGGGUGUUGAGGUCGGACACAGGGCCGGG
CUGGAUGACGGUGAUGCGCGGCUACAGCGAGUGCG
GCGAUGGCUCGCCGGCCGUGUACACGUGCGUGGAC
GACCUGUGCCGCGGCUACGACCUCACGCGACUGUC
AUACGGGCGCAGCAUCUUCACGGAACACGUGUUAG
GCUUCGAGCUGGUGCCACCGUCUCUCUUUAACGUG
GUGGUGGCCAUACGCAACGAAGCCACGCGUACCAA
CCGCGCCGUGCGUCUGCCCGUGAGCACCGCUGCCG
CGCCCGAGGGCAUCACGCUCUUUUACGGCCUGUAC
AACGCAGUGAAGGAAUUCUGCCUGCGUCACCAGCU
GGACCCGCCGCUGCUACGCCACCUAGAUAAAUAGU
ACGCCGGACUGCCGCCCGAGCUGAAGCAGACGCGC
GUCAACCUGCCGGCUCACUCGCGCUAUGGCCCUCA
AGCAGUGGAUGCUCGCUGAUAAUAGGCUGGAGCCU
CGGUGGCCAUGCUUCUUGCCCCUUGGGCCUCCCCC
CAGCCCGUCCUCCCCUUCCUGCACCCGUACCCCCG
UGGUCUUUGAAUAAAGUCUGAGUGGGCGGC
hCMV UL128 penta
(SEQ ID NO: 22)
TCAAGCTTTTGGACCCTCGTACAGAAGCTAATACG
ACTCACTATAGGGTTCGGCTGGTACAGGCTAACCA
GAAGACAGATAAGAGCCTCCATGAGTCCCAAAGAT
CTGACGGCGTTCTTGACGGCGTTGTGGCTGCTATT
GGGTCACAGCCGCGTGCCGCGGGTGCGCGCAGAAG
AATGTTGCGAATTCATAAACGTCAACCACCCGCCG
GAACGCTGTTACGATTTCAAAATGTGCAATCGCTT
CACCGTCGCGCTGCGGTGTCCGGACGGCGAAGTCT
GCTACAGTCCCGAGAAAACGGCTGAGATTCGCGGG
ATCGTCACCACCATGACCCATTCATTGACACGCCA
GGTCGTACACAACAAACTGACGAGCTGCAACTACA
ATCCGTTATACCTCGAAGCTGACGGGCGAATACGC
TGCGGCAAAGTAAACGACAAGGCGCAGTACCTGCT
GGGCGCCGCTGGCAGCGTTCCCTATCGATGGATCA
ATCTGGAATACGACAAGATAACCCGGATCGTGGGC
CTGGATCAGTACCTGGAGAGCGTTAAGAAACACAA
ACGGCTGGATGTGTGCCGCGCTAAAATGGGCTATA
TGCTGCAGTGATAATAGGCTGGAGCCTCGGTGGCC
ATGCTTCTTGCCCCTTGGGCCTCCCCCCAGCCCCT
CCTCCCCTTCCTGCACCCGTACCCCCGTGGTCTTT
GAATAAAGTCTGAGTGGGCGGC
hCMV UL128 penta mRNA
(SEQ ID NO: 179)
UCAAGCUUUUGGACCCUCGUACAGAAGCUAAUACG
ACUCACUAUAGGGUUCGGCUGGUACAGGCUAACCA
GAAGACAGAUAAGAGCCUCCAUGAGUCCCAAAGAU
CUGACGCCGUUCUUGACGGCGUUGUGGCUGCUAUU
GGGUCACAGCCGCGUGCCGCGGGUGCGCGCAGAAG
AAUGUUGCGAAUUCAUAAACGUCAACCACCCGCCG
GAACGCUGUUACGAUUUCAAAAUGUGCAAUCGCUU
CACCGUCGCGCUGCGGUGUCCGGACGGCGAAGUCU
GCUACAGUCCCGAGAAAACGGCUGAGAUUCGCGGG
AUCGUCACCACCAUGACCCAUUCAUUGACACGCCA
GGUCGUACACAACAAACUGACGAGCUGCAACUACA
AUCCGUUAUACCUCGAAGCUGACGGGCGAAUACGC
UGCGGCAAAGUAAACGACAAGGCGCAGUACCUGCU
GGGCGCCGCUGGCAGCGUUCCCUAUCGAUGGAUCA
AUCUGGAAUACGACAAGAUAACCCGGAUCGUGGGC
CUGGAUCAGUACCUGGAGAGCGUUAAGAAACACAA
ACGGCUGGAUGUGUGCCGCGCUAAAAUGGGCUAUA
UGCUGCAGUGAUAAUAGGCUGGAGCCUCGGUGGCC
AUGCUUCUUGCCCCUUGGGCCUCCCCCCAGCCCCU
CCUCCCCUUCCUGCACCCGUACCCCCGUGGUCUUU
GAAUAAAGUCUGAGUGGGCGGC
hCMV-UL130 penta
(SEQ ID NO: 23)
TCAAGCTTTTGGACCCTCGTACAGAAGCTAATACG
ACTCACTATAGGGAGGCTCTTATCTGTCTTCTCAG
TCCGAATTCGAAGTACGGCTACCATGCTGCGGCTT
CTGCTTCGTCACCACTTTCACTGCCTGCTTCTGTG
CGCGGTTTGGGCAACGCCCTGTCTGGCGTCTCCGT
GGTCGACGCTAACAGCAAACCAGAATCCGTCCCGG
CCATGGTCTAAACTGACGTATTCCAAACCGCATGA
CGCGGCGACGTTTTACTGTCCTTTTCTCTATCCCT
CGCCCCCACGATCCCCCTTGCAATTCTCGGGGTTC
CAGCGGGTATCAACGGGTCCCGAGTGTGGCAACGA
GACCCTGTATCTGCTGTACAACCGGGAAGGCCAGA
CCTTGGTGGAGAGAAGCTCCACCTGGGTGAAAAAG
GTGATCTGGTACCTGAGCGGTCGGAACCAAACCAT
CCTCCAACGGATGCCCCGAACGGCTTCGAAACCGA
GCGACGGAAACGTGCAGATCAGCGTGGAAGACGCC
AAGATTTTTGGAGCGCACATGGTGCCCAAGCAGAC
CAAGCTGCTACGCTTCGTCGTCAACGATGGCACAC
GTTATGAGATGTGTGTGATGAAGCTGGAGAGCTGG
GCTCACGTCTTCCGGGACTACAGCGTGTCTTTTCA
GGTGCGATTGACGTTCACCGAGGCCAATAACCAGA
CTTACACCTTCTGCACCCATCCCAATCTCATCGTT
TGATAATAGGCTGGAGCCTCGGTGGCCATGCTTCT
TGCCCCTTGGGCCTCCCCCCAGCCCCTCCTCCCCT
TCCTGCACCCGTACCCCCGTGGTCTTTGAATAAAG
TCTGAGTGGGCGGC
hCMV-UL130 penta mRNA
(SEQ ID NO: 180)
UCAAGCUUUUGGACCCUCGUACAGAAGCUAAUACG
ACUCACUAUAGGGAGGCUCUUAUCUGUCUUCUCAG
UCCGAAUUCGAAGUACGGCUACGAUGCUGCGGCUU
CUGCUUCGUCACCACUUUCACUGCCUGCUUCUGUG
CGCGGUUUGGGCAACGCCCUGUCUGGCGUCUCCGU
GGUCGACGCUAACAGCAAACCAGAAUCCGUCCCCG
CCAUGGUCUAAACUGACGUAUUCCAAACCGCAUGA
CGCGGCGACGUUUUACUGUCCUUUUCUCUAUCCCU
CGCCCCCACGAUCCCCCUUGCAAUUCUCGGGGUUC
CAGCGGGUAUCAACGGGUCCCGAGUGUCGCAACGA
GACCCUGUAUCUGCUGUACAACCGGGAAGGCCAGA
CCUUGGUGGAGAGAAGCUCCACCUGGGUGAAAAAG
GUGAUCUGGUACCUGAGCGGUCGGAACCAAACCAU
CCUCCAACGGAUGCCCCGAACGGCUUCGAAACCGA
GCGACGGAAACGUGCAGAUCAGCGUGGAAGACGCC
AAGAUUUUUGGAGCGCACAUGGUGCCCAAGCAGAC
CAAGCUGCUAGGGUUCGUCGUCAACGAUGGCACAC
GUUAUCAGAUGUGUGUGAUGAAGCUGGAGAGCUGG
GCUGACGUCUUCCGGGACUACAGCGUGUCUUUUCA
GGUGGGAUUGACGUUCACCGAGGCCAAUAACCAGA
CUUACACCUUCUGCACCCAUCCCAAUCUCAUCGUU
UGAUAAUAGGCUGGAGCCUCGGUGGCCAUGCUUCU
UGCCCCUUGGGCCUCCCCCCAGCCCCUCCUCCCCU
UCCUGCACCCGUACCCCCGUGGUCUUUGAAUAAAG
UCUGAGUGGGCGGC
hCMVUL130 trimer
(SEQ ID NO: 24)
TCAAGCTTTTGGACCCTCGTACAGAAGCTAATACG
ACTCACTATAGGGTGGCTCTTATATTTCTTCTTAG
TCCGAATTCGAAGTACGGCTACATGCTGGGGCTTC
TGCTTCGTCACCACTTTCACTGCCTGCTTCTGTGC
GCGGTTTGGGCAACGCCCTGTCTGGCGTCTCCGTG
GTCGACGCTAACAGCAAACCAGAATCCGTCCCCGC
CATGGTCTAAACTGACGTATTCCAAACCGCATGAC
GCGGCGACGTTTTACTGTCCTTTTCTCTATCCCTC
GCCCCCACGATCCCCCTTGCAATTCTCGGGGTTCC
AGCGGGTATCAACGGGTCCCGAGTGTCGCAACGAG
ACCCTGTATCTGCTGTACAACCGGGAAGGCCAGAC
CTTGGTGGAGAGAAGCTCGACCTGGGTGAAAAAGG
TGATGTGGTACCTGAGCGGTCGGAACGAAACCATC
CTCCAACGGATGCCCCGAACGGCTTCGAAACCGAG
CGACGGAAAGGTGCAGATCAGCGTGGAAGACGCCA
AGATTTTTGGAGCGCAGATGGTGCCCAAGCAGACC
AAGCTGCTACGCTTCGTCGTCAACGATGGCACAGG
TTATCAGATGTGTGTGATGAAGCTGGAGAGCTGGG
CTCACGTCTTCCGGGACTACAGCGTGTCTTTTCAG
GTGCGATTGAGGTTCACCGAGGCCAATAACCAGAC
TTACACCTTCTGCACCCATCCCAATCTCATCGTTT
GATAATAGGCTGGAGCCTCGGTGGCCATGCTTCTT
GCCCCTTGGGCCTCCCCCCAGCCCCTCCTCCCCTT
CCTGCACCCGTACCCCCGTGGTCTTTGAATAAAGT
CTGAGTGGGCGGC
hCMVUL130 trimer mRNA
(SEQ ID NO: 181)
UCAAGCUUUUGGACCCUCGUACAGAAGCUAAUACG
ACUCACUAUAGGGUGGCUCUUAUAUUUCUUCUUAG
UCCGAAUUCGAAGUACGGCUACAUGCUGCGGCUUC
UGCUUCGUCACCACUUUCACUGCCUGCUUCUGUGC
GCGGUUUGGGCAACGCCCUGUCUGGCGUCUCCGUG
GUCGACGCUAACAGCAAACCAGAAUCCGUCCCCGC
CAUGGUCUAAACUGACGUAUUCCAAACCGCAUGAC
GCGGCGACGUUUUACUGUCCUUUUCUCUAUCCCUC
GCCCCCACGAUCCCCCUUGCAAUUCUCGGGGUUCC
AGCGGGUAUCAACGGGUCCCGAGUGUCGCAACGAG
ACCCUGUAUCUGCUGUACAACCGGGAAGGCCAGAC
CUUGGUGGAGAGAAGCUCCACCUGGGUGAAAAAGG
UGAUCUGGUACCUGAGCGGUCGGAACCAAACCAUC
CUCCAACGGAUGCCCCGAACGGCUUCGAAACCGAG
CGACGGAAACGUGCAGAUCAGCGUGGAAGACGCCA
AGAUUUUUGGAGCGCACAUGGUGCCCAAGCAGACC
AAGUUGCUACGCUUCGUCGUCAACGAUGGUACACG
UUAUCAGAUGUGUGUGAUGAAGCUGGAGAGCUGGG
CUCACGUCUUCCGGGACUACAGCGUGUCUUUUCAG
GUGCGAUUGACGUUCACCGAGGCCAAUAACCAGAC
UUACACCUUCUGCACCCAUCCCAAUCUCAUCGUUU
GAUAAUAGGCUGGAGCCUCGGUGGCCAUGCUUCUU
GCCCCUUGGGCCUCCCCCCAGCCCCUCCUCCCCUU
CCUGCACCCGUACCCCCGUGGUCUUUGAAUAAAGU
CUGAGUGGGCGGC
hCMV-UL131A penta
(SEQ ID NO: 25)
TCAAGCTTTTGGACCCTCGTACAGAAGCTAATACG
ACTCACTATAGGGTAGCCGTACTTCGAATTCGGAC
AAGCTTCTCTCTCGTCTGTCCATGCGGCTGTGTCG
GGTGTGGCTGTCTGTTTGTCTGTGCGCCGTGGTGC
TGGGTCAGTGCCAGCGGGAAACCGCGGAAAAAAAC
GATTATTACCGAGTACCGCATTACTGGGACGCGTG
CTCTCGCGCGCTGCCCGACCAAACCCGTTACAAGT
ATGTGGAACAGCTCGTGGACCTCACGTTGAACTAC
CACTACGATGCGAGCCACGGCTTGGACAACTTTGA
CGTGCTCAAGAGAATCAACGTGACCGAGGTGTCGT
TGCTCATCAGCGACTTTAGACGTCAGAACCGTCGC
GGCGGCACCAACAAAAGGACCACGTTCAACGCCGC
CGGTTCGCTGGCGCCACACGCCCGGAGCCTCGAGT
TCAGCGTGCGGCTCTTTGCCAACTGATAATAGGCT
GGAGCCTCGGTGGCCATGCTTCTTGCCCCTTGGGC
CTCCCCCCAGCCCCTCCTCCCCTTCCTGCACCCGT
ACCCCCGTGGTCTTTGAATAAAGTCTGAGTGGGCG
GC
hCMV-UL131A penta mRNA
(SEQ ID NO: 182)
UCAAGCUUUUGGACCCUCGUACAGAAGCUAAUACG
ACUCACUAUAGGGUAGCCGUACUUCGAAUUCGGAC
AAGCUUCUCUCUCGUCUGUCCAUGCGGCUGUGUCG
CGUGUGGCUGUCUGUUUGUCUGUGCGCCGUGGUGC
UGGGUCAGUGCCAGCGGGAAACCGCGGAAAAAAAG
GAUUAUUACCGAGUACCGCAUUACUGGGACGCGUG
CUCUCGCGCGCUGCCCGACCAAACCCGUUACAAGU
AUGUGGAACAGCUCGUGGACCUCACGUUGAACUAC
CACUACGAUGCGAGCCACGGCUUGGACAACUUUGA
CGUGCUCAAGAGAAUCAACGUGACCGAGGUGUCGU
UGCUCAUCAGCGACUUUAGACGUCAGAACCGUCGC
GGCGGCACCAACAAAAGGAGCAGGUUCAACGCCGC
CGGUUCGCUGGCGCCACACGCCCGGAGCCUCGAGU
UCAGCGUGCGGCUCUUUGCCAACUGAUAAUAGGCU
GGAGCCUCGGUGGCCAUGCUUCUUGCCCCUUGGGC
CUCCCCCCAGCCCCUCCUCCCCUUCCUGCACCCGU
ACCCCCGUGGUCUUUGAAUAAAGUCUGAGUGGGCG
GC
hCMVUL131A trimer
(SEQ ID NO: 26)
TCAAGCTTTTGGACCCTCGTACAGAAGCTAATACG
ACTCACTATAGGGTAGCCGTACTTCGAATTCGGAC
TTTCTTTTCTCTCTTATTTCCATGCGGCTGTGTCG
GGTGTGGCTGTCTGTTTGTCTGTGCGCCGTGGTGC
TGGGTCAGTGCCAGCGGGAAACCGCGGAAAAAAAC
GATTATTACCGAGTACCGCATTACTGGGACGCGTG
CTCTCGCGCGCTGCCCGACCAAACCCGTTACAAGT
ATGTGGAACAGCTCGTGGACCTCACGTTGAACTAC
CACTACGATGCGAGCCACGGCTTGGACAACTTTGA
CGTGCTCAAGAGAATCAACGTGAGCGAGGTGTCGT
TGCTCATCAGCGACTTTAGACGTCAGAACCGTCGC
GGCGGCACCAACAAAAGGACCACGTTCAACGCCGC
CGGTTCGCTGGCGCCACACGCCCGGAGCCTCGAGT
TCAGCGTGCGGCTCTTTGCCAACTGATAATAGGCT
GGAGCCTCGGTGGCCATGCTTCTTGCCCCTTGGGC
CTCCCCCCAGCCCCTCCTCCCCTTCCTGCACCCGT
ACCCCCGTGGTCTTTGAATAAAGTCTGAGTGGGCG
GC
hCMVUL131A trimer mRNA
(SEQ ID NO: 183)
UCAAGCUUUUGGACCCUCGUACAGAAGCUAAUACG
ACUCACUAUAGGGUAGCCGUACUUCGAAUUCGGAC
UUUCUUUUCUCUCUUAUUUCCAUGCGGCUGUGUCG
GGUGUGGCUGUCUGUUUGUCUGUGCGCCGUGGUGC
UGGGUCAGUGCCAGCGGGAAACCGCGGAAAAAAAC
GAUUAUUACCGAGUACCGCAUUACUGGGACGCGUG
CUCUCGCGCGCUGCCCGACCAAACCCGUUACAAGU
AUGUGGAACAGCUCGUGGACCUCACGUUGAACUAC
CACUACGAUGCGAGCCACGGCUUGGACAACUUUGA
CGUGCUCAAGAGAAUCAACGUGACCGAGGUGUCGU
UGCUCAUCAGCGACUUUAGACGUCAGAACCGUCGC
GGCGGCACCAACAAAAGGACCACGUUCAACGCCGC
CGGUUCGCUGGCGCCACACGCCCGGAGCCUCGAGU
UCAGCGUGCGGCUCUUUGCCAACUGAUAAUAGGCU
GGAGCCUCGGUGGCCAUGCUUCUUGCCCCUUGGGC
CUCCCCCCAGCCCCUCCUCCCCUUCCUGCACCCGU
ACCCCCGUGGUCUUUGAAUAAAGUCUGAGUGGGCG
GC

### Example 16: hCMV Vaccine—hCMV UL Fusion Sequences

A hCMV vaccine may comprise, for example, at least one RNA polynucleotide encoded by at least one of the following sequences or by at least one fragment or epitope of the following sequences. In some embodiments, a hCMV vaccine may comprise at least one RNA polynucleotide comprising at least one of the mRNA sequences listed below or at least one fragment of the mRNA sequences listed below.

hCMV pp65-IE1, hCMV UL83-UL123 fusion
(SEQ ID NO: 27)
TCAAGCTTTTGGACCCTCGTACAGAAGCTAATACG
ACTCACTATAGGGAAATAAGAGAGAAAAGAAGAGT
AAGAAGAAATATAAGAGCCACCATGGAGTCGCGCG
GTCGCCGTTGTCCCGAAATGATATCCGTACTGGGT
CCCATTTCGGGGCACGTGCTGAAAGCCGTGTTTAG
TCGCGGCGATACGCCGGTGCTGCCGCACGAGACGC
GACTCCTGCAGACGGGTATCCACGTACGCGTGAGC
CAGCCCTCGCTGATCCTGGTGTCGCAGTACACGCC
CGACTCGACGCCATGCCACCGCGGCGACAATCAGC
TGCAGGTGCAGCACACGTACTTTACGGGCAGCGAG
GTGGAGAACGTGTCGGTCAACGTGCACAACCCCAC
GGGCCGAAGCATCTGCCCCAGCCAAGAGCCCATGT
CGATCTATGTGTACGCGCTGCCGCTCAAGATGCTG
AACATCCCCAGCATCAACGTGCACCACTACCCGTC
GGCGGCCGAGCGCAAACACCGACACCTGCCCGTAG
CCGACGCTGTTATTCACGCGTCGGGCAAGCAGATG
TGGCAGGCGCGTCTCACGGTCTCGGGACTGGCCTG
GACGCGTCAGCAGAACCAGTGGAAAGAGCCCGACG
TCTACTACACGTCAGCGTTCGTGTTTCCCACCAAG
GACGTGGCACTGCGGCACGTGGTGTGCGCGCACGA
GCTGGTTTGCTCCATGGAGAACACGCGCGCAACCA
AGATGCAGGTGATAGGTGACCAGTACGTCAAGGTG
TACCTGGAGTCCTTCTGCGAGGACGTGCCCTCCGG
CAAGCTCTTTATGCACGTCACGCTGGGCTCTGACG
TGGAAGAGGACCTAACGATGACCCGCAACCCGCAA
CCCTTCATGCGCCCCCACGAGCGCAACGGCTTTAC
GGTGTTGTGTCCCAAAAATATGATAATCAAACCGG
GCAAGATCTCGCACATCATGCTGGATGTGGCTTTT
ACCTCACACGAGCATTTTGGGCTGCTGTGTCCCAA
GAGCATCCCGGGCCTGAGCATCTCAGGTAACCTGT
TGATGAACGGGCAGCAAATCTTCCTGGAGGTACAA
GCGATACGCGAGACCGTGGAACTGCGTCAGTACGA
TCCCGTGGCTGCGCTCTTCTTTTTCGATATCGACT
TGTTGCTGCAGCGCGGGCCTCAGTACAGCGAGCAC
CCCACCTTCACCAGCCAGTATCGCATCCAGGGCAA
GCTTGAGTACCGACACACCTGGGACCGGCACGACG
AGGGTGCCGCCCAGGGCGACGACGACGTCTGGACC
AGCGGATCGGACTCCGACGAAGAACTCGTAACCAC
CGAGCGTAAGACGCCCCGCGTCACCGGCGGCGGCG
CCATGGCGAGCGCCTCCACTTCCGCGGGCCGCAAA
CGCAAATCAGCATCCTCGGCGACGGCGTGCACGGC
GGGCGTTATGACACGCGGCCGCCTTAAGGCCGAGT
CCACCGTCGCGCCCGAAGAGGACACCGACGAGGAT
TCCGACAACGAAATCCACAATCCGGCCGTGTTCAC
CTGGCCGCCCTGGCAGGCCGGCATCCTGGCCCGCA
ACCTGGTGCCCATGGTGGCTACGGTTCAGGGTCAG
AATCTGAAGTACCAGGAGTTCTTCTGGGACGCCAA
CGACATCTACCGCATCTTCGCCGAATTGGAAGGCG
TATGGCAGCCCGCTGCGCAACCCAAACGTCGCCGC
CACCGGCAAGACGCCTTGCCCGGGCCATGCATCGC
CTCGACGCCCAAAAAGCACCGAGGTGAGTCCTCTG
CCAAGAGAAAGATGGACCCTGATAATCCTGACGAG
GGCCCTTCCTCCAAGGTGCCACGGCCCGAGACACC
CGTGACCAAGGCCACGACGTTCCTGCAGACTATGT
TAAGGAAGGAGGTTAACAGTCAGCTGAGCCTGGGA
GACCCGCTGTTCCCAGAATTGGCCGAAGAATCCCT
CAAAACCTTTGAACAAGTGACCGAGGATTGCAACG
AGAACCCCGAAAAAGATGTCCTGACAGAACTCGTC
AAACAGATTAAGGTTCGAGTGGACATGGTGCGGCA
TAGAATCAAGGAGCACATGCTGAAAAAATATACCC
AGACGGAAGAAAAATTCACTGGCGCCTTTAATATG
ATGGGAGGATGTTTGCAGAATGCCTTAGATATCTT
AGATAAGGTTCATGAGCCTTTCGAGGACATGAAGT
GTATTGGGCTAACTATGCAGAGCATGTATGAGAAC
TACATTGTACCTGAGGATAAGCGGGAGATGTGGAT
GGCTTGTATTAAGGAGCTGCATGATGTGAGCAAGG
GCGCCGCTAACAAGTTGGGGGGTGCACTGCAGGCT
AAGGCCCGTGCTAAAAAGGATGAACTTAGGAGAAA
GATGATGTATATGTGCTACAGGAATATAGAGTTCT
TTACCAAGAACTCAGCCTTCCCTAAGACCACCAAT
GGCTGCAGTCAGGCCATGGCGGCATTGCAGAACTT
GCCTCAGTGCTCTCCTGATGAGATTATGTCTTATG
CCCAGAAAATCTTTAAGATTTTGGATGAGGAGAGA
GACAAGGTGCTCACGCACATTGATCACATATTTAT
GGATATCCTCACTACATGTGTGGAAACAATGTGTA
ATGAGTACAAGGTCACTAGTGACGCTTGTATGATG
ACCATGTACGGGGGCATCTCTCTCTTAAGTGAGTT
CTGTCGGGTGCTGTGCTGCTATGTCTTAGAGGAGA
CTAGTGTGATGCTGGCCAAGCGGCCTCTGATAACC
AAGCCTGAGGTTATCAGTGTAATGAAGCGCCGCAT
TGAGGAGATCTGCATGAAGGTCTTTGCCCAGTACA
TTCTGGGGGCCGATCCTTTGAGAGTCTGCTCTCCT
AGTGTGGATGACCTACGGGCCATCGCCGAGGAGTC
AGATGAGGAAGAGGCTATTGTAGCCTACACTTTGG
CCACCGCTGGTGCCAGCTCCTCTGATTCTCTGGTG
TCACCTCCAGAGTCCCCTGTACCCGCGACTATCCC
TCTGTCCTCAGTAATTGTGGCTGAGAACAGTGATC
AGGAAGAAAGTGAACAGAGTGATGAGGAACAGGAG
GAGGGTGCTCAGGAGGAGCGGGAGGACACTGTGTC
TGTCAAGTCTGAGCCAGTGTCTGAGATAGAGGAAG
TTGCCTCAGAGGAAGAGGAGGATGGTGCTGAGGAA
CCCACCGCCTCTGGAGGCAAGAGCACCCACCCTAT
GGTGACTAGAAGCAAGGCTGACCAGTGATAATAGG
CTGGAGCCTCGGTGGCCATGCTTCTTGCCCCTTGG
GCCTCCCCCCAGCCCCTCCTCCCCTTCCTGCACCC
GTACCCCCGTGGTCTTTGAATAAAGTCTGAGTGGG
CGGC
hCMV pp65-IE1, hCMV UL83-UL123
fusion mRNA
(SEQ ID NO: 184)
UCAAGCUUUUGGACCCUCGUACAGAAGCUAAUACG
ACUCACUAUAGGGAAAUAAGAGAGAAAAGAAGAGU
AAGAAGAAAUAUAAGAGCCACCAUGGAGUCGCGCG
GUCGCCGUUGUCCCGAAAUGAUAUCCGUACUGGGU
CCCAUUUCGGGGCACGUGCUGAAAGCCGUGUUUAG
UCGCGGCGAUACGCCGGUGCUGCCGCACGAGACGC
GACUCCUGCAGACGGGUAUCCACGUACGCGUGAGC
CAGCCCUCGCUGAUCCUGGUGUCGCAGUACACGCC
CGACUCGACGCCAUGCCACCGCGGCGACAAUCAGC
UGCAGGUGCAGCACACGUACUUUACGGGCAGCGAG
GUGGAGAACGUGUCGGUCAACGUGCACAACCCCAC
GGGCCGAAGCAUCUGCCCCAGCCAAGAGCCCAUGU
CGAUCUAUGUGUACGCGCUGCCGCUCAAGAUGCUG
AACAUCCCCAGCAUCAACGUGCACCACUACCCGUC
GGCGGCCGAGCGCAAACACCGACACCUGCCCGUAG
CCGACGCUGUUAUUCACGCGUCGGGCAAGCAGAUG
UGGCAGGCGCGUCUCACGGUCUCGGGACUGGCCUG
GACGCGUCAGCAGAACCAGUGGAAAGAGCCCGACG
UCUACUACACGUCAGCGUUCGUGUUUCCCACCAAG
GACGUGGCACUGCGGCACGUGGUGUGCGCGCACGA
GCUGGUUUGCUCCAUGGAGAACACGCGCGCAACCA
AGAUGCAGGUGAUAGGUGACCAGUACGUCAAGGUG
UACCUGGAGUCCUUCUGCGAGGACGUGCCCUCCGG
CAAGCUCUUUAUGCACGUCACGCUGGGCUCUGACG
UGGAAGAGGACCUAACGAUGACCCGCAACCCGCAA
CCCUUCAUGCGCCCCCACGAGCGCAACGGCUUUAC
GGUGUUGUGUCCCAAAAAUAUGAUAAUCAAACCGG
GCAAGAUCUCGCACAUCAUGCUGGAUGUGGCUUUU
ACCUCACACGAGCAUUUUGGGCUGCUGUGUCCCAA
GAGCAUCCCGGGCCUGAGCAUCUCAGGUAACCUGU
UGAUGAACGGGCAGCAAAUCUUCCUGGAGGUACAA
GCGAUACGCGAGACCGUGGAACUGCGUCAGUACGA
UCCCGUGGCUGCGCUCUUCUUUUUCGAUAUCGACU
UGUUGCUGCAGCGCGGGCCUCAGUACAGCGAGCAC
CCCACCUUCACCAGCCAGUAUCGCAUCCAGGGCAA
GCUUGAGUACCGACACACCUGGGACCGGCACGACG
AGGGUGCCGCCCAGGGCGACGACGACGUCUGGACC
AGCGGAUCGGACUCCGACGAAGAACUCGUAACCAC
CGAGCGUAAGACGCCCCGCGUCACCGGCGGCGGCG
CCAUGGCGAGCGCCUCCACUUCCGCGGGCCGCAAA
CGCAAAUCAGCAUCCUCGGCGACGGCGUGCACGGC
GGGCGUUAUGACACGCGGCCGCCUUAAGGCCGAGU
CCACCGUCGCGCCCGAAGAGGACACCGACGAGGAU
UCCGACAACGAAAUCCACAAUCCGGCCGUGUUCAC
CUGGCCGCCCUGGCAGGCCGGCAUCCUGGCCCGCA
ACCUGGUGCCCAUGGUGGCUACGGUUCAGGGUCAG
AAUCUGAAGUACCAGGAGUUCUUCUGGGACGCCAA
CGACAUCUACCGCAUCUUCGCCGAAUUGGAAGGCG
UAUGGCAGCCCGCUGCGCAACCCAAACGUCGCCGC
CACCGGCAAGACGCCUUGCCCGGGCCAUGCAUCGC
CUCGACGCCCAAAAAGCACCGAGGUGAGUCCUCUG
CCAAGAGAAAGAUGGACCCUGAUAAUCCUGACGAG
GGCCCUUCCUCCAAGGUGCCACGGCCCGAGACACC
CGUGACCAAGGCCACGACGUUCCUGCAGACUAUGU
UAAGGAAGGAGGUUAACAGUCAGCUGAGCCUGGGA
GACCCGCUGUUCCCAGAAUUGGCCGAAGAAUCCCU
CAAAACCUUUGAACAAGUGACCGAGGAUUGCAACG
AGAACCCCGAAAAAGAUGUCCUGACAGAACUCGUC
AAACAGAUUAAGGUUCGAGUGGACAUGGUGCGGCA
UAGAAUCAAGGAGCACAUGCUGAAAAAAUAUACCC
AGACGGAAGAAAAAUUCACUGGCGCCUUUAAUAUG
AUGGGAGGAUGUUUGCAGAAUGCCUUAGAUAUCUU
AGAUAAGGUUCAUGAGCCUUUCGAGGACAUGAAGU
GUAUUGGGCUAACUAUGCAGAGCAUGUAUGAGAAC
UACAUUGUACCUGAGGAUAAGCGGGAGAUGUGGAU
GGCUUGUAUUAAGGAGCUGCAUGAUGUGAGCAAGG
GCGCCGCUAACAAGUUGGGGGGUGCACUGCAGGCU
AAGGCCCGUGCUAAAAAGGAUGAACUUAGGAGAAA
GAUGAUGUAUAUGUGCUACAGGAAUAUAGAGUUCU
UUACCAAGAACUCAGCCUUCCCUAAGACCACCAAU
GGCUGCAGUCAGGCCAUGGCGGCAUUGCAGAACUU
GCCUCAGUGCUCUCCUGAUGAGAUUAUGUCUUAUG
CCCAGAAAAUCUUUAAGAUUUUGGAUGAGGAGAGA
GACAAGGUGCUCACGCACAUUGAUCACAUAUUUAU
GGAUAUCCUCACUACAUGUGUGGAAACAAUGUGUA
AUGAGUACAAGGUCACUAGUGACGCUUGUAUGAUG
ACCAUGUACGGGGGCAUCUCUCUCUUAAGUGAGUU
CUGUCGGGUGCUGUGCUGCUAUGUCUUAGAGGAGA
CUAGUGUGAUGCUGGCCAAGCGGCCUCUGAUAACC
AAGCCUGAGGUUAUCAGUGUAAUGAAGCGCCGCAU
UGAGGAGAUCUGCAUGAAGGUCUUUGCCCAGUACA
UUCUGGGGGCCGAUCCUUUGAGAGUCUGCUCUCCU
AGUGUGGAUGACCUACGGGCCAUCGCCGAGGAGUC
AGAUGAGGAAGAGGCUAUUGUAGCCUACACUUUGG
CCACCGCUGGUGCCAGCUCCUCUGAUUCUCUGGUG
UCACCUCCAGAGUCCCCUGUACCCGCGACUAUCCC
UCUGUCCUCAGUAAUUGUGGCUGAGAACAGUGAUC
AGGAAGAAAGUGAACAGAGUGAUGAGGAACAGGAG
GAGGGUGCUCAGGAGGAGCGGGAGGACACUGUGUC
UGUCAAGUCUGAGCCAGUGUCUGAGAUAGAGGAAG
UUGCCUCAGAGGAAGAGGAGGAUGGUGCUGAGGAA
CCCACCGCCUCUGGAGGCAAGAGCACCCACCCUAU
GGUGACUAGAAGCAAGGCUGACCAGUGAUAAUAGG
CUGGAGCCUCGGUGGCCAUGCUUCUUGCCCCUUGG
GCCUCCCCCCAGCCCCUCCUCCCCUUCCUGCACCC
GUACCCCCGUGGUCUUUGAAUAAAGUCUGAGUGGG
CGGC
hCMV PP65-IE1FLAG, hCMV UL83-UL123
FLAG tag
(SEQ ID NO: 28)
TCAAGCTTTTGGACCCTCGTACAGAAGCTAATACG
ACTCACTATAGGGAAATAAGAGAGAAAAGAAGAGT
AAGAAGAAATATAAGAGCCACCATGGAGTCGCGCG
GTCGCCGTTGTCCCGAAATGATATCCGTACTGGGT
CCCATTTCGGGGCACGTGCTGAAAGCCGTGTTTAG
TCGCGGCGATACGCCGGTGCTGCCGCACGAGACGC
GACTCCTGCAGACGGGTATCCACGTACGCGTGAGC
CAGCCCTCGCTGATCCTGGTGTCGCAGTACACGCC
CGACTCGACGCCATGCCACCGCGGCGACAATCAGC
TGCAGGTGCAGCACACGTACTTTACGGGCAGCGAG
GTGGAGAACGTGTCGGTCAACGTGCACAACCCCAC
GGGCCGAAGCATCTGCCCCAGCCAAGAGCCCATGT
CGATCTATGTGTACGCGCTGCCGCTCAAGATGCTG
AACATCCCCAGCATCAACGTGCACCACTACCCGTC
GGCGGCCGAGCGCAAACACCGACACCTGCCCGTAG
CCGACGCTGTTATTCACGCGTCGGGCAAGCAGATG
TGGCAGGCGCGTCTCACGGTCTCGGGACTGGCCTG
GACGCGTCAGCAGAACCAGTGGAAAGAGCCCGACG
TCTACTACACGTCAGCGTTCGTGTTTCCCACCAAG
GACGTGGCACTGCGGCACGTGGTGTGCGCGCACGA
GCTGGTTTGCTCCATGGAGAACACGCGCGCAACCA
AGATGCAGGTGATAGGTGACCAGTACGTCAAGGTG
TACCTGGAGTCCTTCTGCGAGGACGTGCCCTCCGG
CAAGCTCTTTATGCACGTCACGCTGGGCTCTGACG
TGGAAGAGGACCTAACGATGACCCGCAACCCGCAA
CCCTTCATGCGCCCCCACGAGCGCAACGGCTTTAC
GGTGTTGTGTCCCAAAAATATGATAATCAAACCGG
GCAAGATCTCGCACATCATGCTGGATGTGGCTTTT
ACCTCACACGAGCATTTTGGGCTGCTGTGTCCCAA
GAGCATCCCGGGCCTGAGCATCTCAGGTAACCTGT
TGATGAACGGGCAGCAAATCTTCCTGGAGGTACAA
GCGATACGCGAGACCGTGGAACTGCGTCAGTACGA
TCCCGTGGCTGCGCTCTTCTTTTTCGATATCGACT
TGTTGCTGCAGCGCGGGCCTCAGTACAGCGAGCAC
CCCACCTTCACCAGCCAGTATCGCATCCAGGGCAA
GCTTGAGTACCGACACACCTGGGACCGGCACGACG
AGGGTGCCGCCCAGGGCGACGACGACGTCTGGACC
AGCGGATCGGACTCCGACGAAGAACTCGTAACCAC
CGAGCGTAAGACGCCCCGCGTCACCGGCGGCGGCG
CCATGGCGAGCGCCTCCACTTCCGCGGGCCGCAAA
CGCAAATCAGCATCCTCGGCGACGGCGTGCACGGC
GGGCGTTATGACACGCGGCCGCCTTAAGGCCGAGT
CCACCGTCGCGCCCGAAGAGGACACCGACGAGGAT
TCCGACAACGAAATCCACAATCCGGCCGTGTTCAC
CTGGCCGCCCTGGCAGGCCGGCATCCTGGCCCGCA
ACCTGGTGCCCATGGTGGCTACGGTTCAGGGTCAG
AATCTGAAGTACCAGGAGTTCTTCTGGGACGCCAA
CGACATCTACCGCATCTTCGCCGAATTGGAAGGCG
TATGGCAGCCCGCTGCGCAACCCAAACGTCGCCGC
CACCGGCAAGACGCCTTGCCCGGGCCATGCATCGC
CTCGACGCCCAAAAAGCACCGAGGTGAGTCCTCTG
CCAAGAGAAAGATGGACCCTGATAATCCTGACGAG
GGCCCTTCCTCCAAGGTGCCACGGCCCGAGACACC
CGTGACCAAGGCCACGACGTTCCTGCAGACTATGT
TAAGGAAGGAGGTTAACAGTCAGCTGAGCCTGGGA
GACCCGCTGTTCCCAGAATTGGCCGAAGAATCCCT
CAAAACCTTTGAACAAGTGACCGAGGATTGCAACG
AGAACCCCGAAAAAGATGTCCTGACAGAACTCGTC
AAACAGATTAAGGTTCGAGTGGACATGGTGCGGCA
TAGAATCAAGGAGCACATGCTGAAAAAATATACCC
AGACGGAAGAAAAATTCACTGGCGCCTTTAATATG
ATGGGAGGATGTTTGCAGAATGCCTTAGATATCTT
AGATAAGGTTCATGAGCCTTTCGAGGACATGAAGT
GTATTGGGCTAACTATGCAGAGCATGTATGAGAAC
TACATTGTACCTGAGGATAAGCGGGAGATGTGGAT
GGCTTGTATTAAGGAGCTGCATGATGTGAGCAAGG
GCGCCGCTAACAAGTTGGGGGGTGCACTGCAGGCT
AAGGCCCGTGCTAAAAAGGATGAACTTAGGAGAAA
GATGATGTATATGTGCTACAGGAATATAGAGTTCT
TTACCAAGAACTCAGCCTTCCCTAAGACCACCAAT
GGCTGCAGTCAGGCCATGGCGGCATTGCAGAACTT
GCCTCAGTGCTCTCCTGATGAGATTATGTCTTATG
CCCAGAAAATCTTTAAGATTTTGGATGAGGAGAGA
GACAAGGTGCTCACGCACATTGATCACATATTTAT
GGATATCCTCACTACATGTGTGGAAACAATGTGTA
ATGAGTACAAGGTCACTAGTGACGCTTGTATGATG
ACCATGTACGGGGGCATCTCTCTCTTAAGTGAGTT
CTGTCGGGTGCTGTGCTGCTATGTCTTAGAGGAGA
CTAGTGTGATGCTGGCCAAGCGGCCTCTGATAACC
AAGCCTGAGGTTATCAGTGTAATGAAGCGCCGCAT
TGAGGAGATCTGCATGAAGGTCTTTGCCCAGTACA
TTCTGGGGGCCGATCCTTTGAGAGTCTGCTCTCCT
AGTGTGGATGACCTACGGGCCATCGCCGAGGAGTC
AGATGAGGAAGAGGCTATTGTAGCCTACACTTTGG
CCACCGCTGGTGCCAGCTCCTCTGATTCTCTGGTG
TCACCTCCAGAGTCCCCTGTACCCGCGACTATCCC
TCTGTCCTCAGTAATTGTGGCTGAGAACAGTGATC
AGGAAGAAAGTGAACAGAGTGATGAGGAACAGGAG
GAGGGTGCTCAGGAGGAGCGGGAGGACACTGTGTC
TGTCAAGTCTGAGCCAGTGTCTGAGATAGAGGAAG
TTGCCTCAGAGGAAGAGGAGGATGGTGCTGAGGAA
CCCACCGCCTCTGGAGGCAAGAGCACCCACCCTAT
GGTGACTAGAAGCAAGGCTGACCAGGATTACAAGG
ACGATGACGATAAGTGATAATAGGCTGGAGCCTCG
GTGGCCATGCTTCTTGCCCCTTGGGCCTCCCCCCA
GCCCCTCCTCCCCTTCCTGCACCCGTACCCCCGTG
GTCTTTGAATAAAGTCTGAGTGGGCGGC
hCMV pp65-IE1, hCMV UL83-UL123
fusion mRNA
(SEQ ID NO: 185)
UCAAGCUUUUGGACCCUCGUACAGAAGCUAAUACG
ACUCACUAUAGGGAAAUAAGAGAGAAAAGAAGAGU
AAGAAGAAAUAUAAGAGCCACCAUGGAGUCGCGCG
GUCGCCGUUGUCCCGAAAUGAUAUCCGUACUGGGU
CCCAUUUCGGGGCACGUGCUGAAAGCCGUGUUUAG
UCGCGGCGAUACGCCGGUGCUGCCGCACGAGACGC
GACUCCUGCAGACGGGUAUCCACGUACGCGUGAGC
CAGCCCUCGCUGAUCCUGGUGUCGCAGUACACGCC
CGACUCGACGCCAUGCCACCGCGGCGACAAUCAGC
UGCAGGUGCAGCACACGUACUUUACGGGCAGCGAG
GUGGAGAACGUGUCGGUCAACGUGCACAACCCCAC
GGGCCGAAGCAUCUGCCCCAGCCAAGAGCCCAUGU
CGAUCUAUGUGUACGCGCUGCCGCUCAAGAUGCUG
AACAUCCCCAGCAUCAACGUGCACCACUACCCGUC
GGCGGCCGAGCGCAAACACCGACACCUGCCCGUAG
CCGACGCUGUUAUUCACGCGUCGGGCAAGCAGAUG
UGGCAGGCGCGUCUCACGGUCUCGGGACUGGCCUG
GACGCGUCAGCAGAACCAGUGGAAAGAGCCCGACG
UCUACUACACGUCAGCGUUCGUGUUUCCCACCAAG
GACGUGGCACUGCGGCACGUGGUGUGCGCGCACGA
GCUGGUUUGCUCCAUGGAGAACACGCGCGCAACCA
AGAUGCAGGUGAUAGGUGACCAGUACGUCAAGGUG
UACCUGGAGUCCUUCUGCGAGGACGUGCCCUCCGG
CAAGCUCUUUAUGCACGUCACGCUGGGCUCUGACG
UGGAAGAGGACCUAACGAUGACCCGCAACCCGCAA
CCCUUCAUGCGCCCCCACGAGCGCAACGGCUUUAC
GGUGUUGUGUCCCAAAAAUAUGAUAAUCAAACCGG
GCAAGAUCUCGCACAUCAUGCUGGAUGUGGCUUUU
ACCUCACACGAGCAUUUUGGGCUGCUGUGUCCCAA
GAGCAUCCCGGGCCUGAGCAUCUCAGGUAACCUGU
UGAUGAACGGGCAGCAAAUCUUCCUGGAGGUACAA
GCGAUACGCGAGACCGUGGAACUGCGUCAGUACGA
UCCCGUGGCUGCGCUCUUCUUUUUCGAUAUCGACU
UGUUGCUGCAGCGCGGGCCUCAGUACAGCGAGCAC
CCCACCUUCACCAGCCAGUAUCGCAUCCAGGGCAA
GCUUGAGUACCGACACACCUGGGACCGGCACGACG
AGGGUGCCGCCCAGGGCGACGACGACGUCUGGACC
AGCGGAUCGGACUCCGACGAAGAACUCGUAACCAC
CGAGCGUAAGACGCCCCGCGUCACCGGCGGCGGCG
CCAUGGCGAGCGCCUCCACUUCCGCGGGCCGCAAA
CGCAAAUCAGCAUCCUCGGCGACGGCGUGCACGGC
GGGCGUUAUGACACGCGGCCGCCUUAAGGCCGAGU
CCACCGUCGCGCCCGAAGAGGACACCGACGAGGAU
UCCGACAACGAAAUCCACAAUCCGGCCGUGUUCAC
CUGGCCGCCCUGGCAGGCCGGCAUCCUGGCCCGCA
ACCUGGUGCCCAUGGUGGCUACGGUUCAGGGUCAG
AAUCUGAAGUACCAGGAGUUCUUUUGGGACGCCAA
CGACAUCUACCGLAUCUUCGCCGAAUUGGAAGGCG
UAUGGCAGCCCGCUGCGCAACCCAAACGUCGCCGC
CACCGGCAAGACGCCUUGCCCGGGCCAUGCAUCGC
CUCGACGCCCAAAAAGCACCGAGGUGAGUCCUCUG
CCAAGAGAAAGAUGGACCCUGAUAAUCCUGACGAG
GGCCCUUCCUCCAAGGUGCCACGGCCCGAGACACC
CGUGACCAAGGCCACGACGUUCCUGCAGACUAUGU
UAAGGAAGGAGGUUAACAGUCAGCUGAGCCUGGGA
GACCCGCUGUUCCCAGAAUUGGCCGAAGAAUCCCU
CAAAACCUUUGAACAAGUGACCGAGGAUUGCAACG
AGAACCCCGAAAAAGAUGUCCUGACAGAACUCGUC
AAACAGAUUAAGGUUCGAGUGGACAUGGUGCGGCA
UAGAAUCAAGGAGCACAUGCUGAAAAAAUAUACCC
AGACGGAAGAAAAAUUCACUGGCGCCUUUAAUAUG
AUGGGAGGAUGUUUGCAGAAUGCCUUAGAUAUCUU
AGAUAAGGUUCAUGAGCCUUUCGAGGACAUGAAGU
GUAUUGGGCUAACUAUGCAGAGCAUGUAUGAGAAC
UACAUUGUACCUGAGGAUAAGCGGGAGAUGUGGAU
GGCUUGUAUUAAGGAGCUGCAUGAUGUGAGCAAGG
GCGCCGCUAACAAGUUGGGGGGUGCACUGCAGGCU
AAGGCCCGUGCUAAAAAGGAUGAACUUAGGAGAAA
GAUGAUGUAUAUGUGCUACAGGAAUAUAGAGUUCU
UUACCAAGAACUCAGCCUUCCCUAAGACCACCAAU
GGCUGCAGUCAGGCCAUGGCGGCAUUGCAGAACUU
GCCUCAGUGCUCUCCUGAUGAGAUUAUGUCUUAUG
CCCAGAAAAUCUUUAAGAUUUUGGAUGAGGAGAGA
GACAAGGUGCUCACGCACAUUGAUCACAUAUUUAU
GGAUAUCCUCACUACAUGUGUGGAAACAAUGUGUA
AUGAGUACAAGGUCACUAGUGACGCUUGUAUGAUG
ACCAUGUACGGGGGCAUCUCUCUCUUAAGUGAGUU
CUGUCGGGUGCUGUGCUGCUAUGUCUUAGAGGAGA
CUAGUGUGAUGCUGGCCAAGCGGCCUCUGAUAACC
AAGCCUGAGGUUAUCAGUGUAAUGAAGCGCCGCAU
UGAGGAGAUCUGCAUGAAGGUCUUUGCCCAGUACA
UUCUGGGGGCCGAUCCUUUGAGAGUCUGCUCUCCU
AGUGUGGAUGACCUACGGGCCAUCGCCGAGGAGUC
AGAUGAGGAAGAGGCUAUUGUAGCCUACACUUUGG
CCACCGCUGGUGCCAGCUCCUCUGAUUCUCUGGUG
UCACCUCCAGAGUCCCCUGUACCCGCGACUAUCCC
UCUGUCCUCAGUAAUUGUGGCUGAGAACAGUGAUC
AGGAAGAAAGUGAACAGAGUGAUGAGGAACAGGAG
GAGGGUGCUCAGGAGGAGCGGGAGGACACUGUGUC
UGUCAAGUCUGAGCCAGUGUCUGAGAUAGAGGAAG
UUGCCUCAGAGGAAGAGGAGGAUGGUGCUGAGGAA
CCCACCGCCUCUGGAGGCAAGAGCACCCACCCUAU
GGUGACUAGAAGCAAGGCUGACCAGUGAUAAUAGG
CUGGAGCCUCGGUGGCCAUGCUUCUUGCCCCUUGG
GCCUCCCCCCAGCCCCUCCUCCCCUUCCUGCACCC
GUACCCCCGUGGUCUUUGAAUAAAGUCUGAGUGGG
CGGC

### Example 17: hCMV Vaccine—hCMV Concatameric Sequences

A hCMV vaccine may comprise, for example, at least one RNA polynucleotide encoded by at least one of the following sequences or by at least one fragment or epitope of the following sequences. In some embodiments, a hCMV vaccine may comprise at least one RNA polynucleotide comprising at least one of the mRNA sequences listed below or at least one fragment of the mRNA sequences listed below.

hCMVgH-2A-gL (ORF-gH-Furing-Linker-P2A-gL)
Furin:
(SEQ ID NO: 148)
CCGCGCCAAGAGGAGC
Linker:
GGAAGCGGA
P2A peptide:
(SEQ ID NO: 149)
GCTACTAACTTCAGCCTGCTGAAGCAGGCTGGAGACGTGGAGGAGAACCCTGGACCT
5′-UTR: bold
3′-UTR: underline
hCMVgH-2A-gL (ORF-gH-Furin-Linker-P2A-gL)
(SEQ ID NO: 29)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCCAGGCCTCCCCTCCTACCTCATCAT
CCTCGCCGTCTGTCTCTTCAGCCACCTACTTTCGTCACGATATGGCGCAGAAGCCGTATCCG
AACCGCTGGACAAAGCGTTTCACCTACTGCTCAACACCTACGGGAGACCCATCCGCTTCCTG
CGTGAAAATACCACCCAGTGTACCTACAACAGCAGCCTCCGTAACAGCACGGTCGTCAGGGA
AAACGCCATCAGTTTCAACTTTTTCCAAAGCTATAATCAATACTATGTATTCCATATGCCTC
GATGTCTTTTTGCGGGTCCTCTGGCGGAGCAGTTTCTGAACCAGGTAGATCTGACCGAAACC
CTGGAAAGATACCAACAGAGACTTAACACTTACGCGCTGGTATCCAAAGACCTGGCCAGCTA
CCGATCTTTTTCGCAGCAGCTAAAGGCACAAGACAGCCTAGGTGAACAGCCCACCACTGTGC
CACCGCCCATTGACCTGTCAATACCTCACGTTTGGATGCCACCGCAAACCACTCCACACGGC
TGGACAGAATCACATACCACCTCAGGACTACACCGACCACACTTTAACCAGACCTGTATCCT
CTTTGATGGACACGATCTACTATTCAGCACCGTCACACCTTGTTTGCACCAAGGCTTTTACC
TCATCGACGAACTACGTTACGTTAAAATAACACTGACCGAGGACTTCTTCGTAGTTACGGTG
TCCATAGACGACGACACACCCATGCTGCTTATCTTCGGCCATCTTCCACGCGTACTTTTCAA
AGCGCCCTATCAACGCGACAACTTTATACTACGACAAACTGAAAAACACGAGCTCCTGGTGC
TAGTTAAGAAAGATCAACTGAACCGTCACTCTTATCTCAAAGACCCGGACTTTCTTGACGCC
GCACTTGACTTCAACTACCTAGACCTCAGCGCACTACTACGTAACAGCTTTCACCGTTACGC
CGTGGATGTACTCAAGAGCGGTCGATGTCAGATGCTGGACCGCCGCACGGTAGAAATGGCCT
TCGCCTACGCATTAGCACTGTTCGCAGCAGCCCGACAAGAAGAGGCCGGCGCCCAAGTCTCC
GTCCCACGGGCCCTAGACCGCCAGGCCGCACTCTTACAAATACAAGAATTTATGATCACCTG
CCTCTCACAAACACCACCACGCACCACGTTGCTGCTGTATCCCACGGCCGTGGACCTGGCCA
AACGAGCCCTTTGGACACCGAATCAGATCACCGACATCACCAGCCTCGTACGCCTGGTCTAC
ATACTCTCTAAACAGAATCAGCAACATCTCATCCCCCAATGGGCACTACGACAGATCGCCGA
CTTTGCCCTAAAACTACACAAAACGCACCTGGCCTCTTTTCTTTCAGCCTTCGCACGCCAAG
AACTCTACCTCATGGGCAGCCTCGTCCACTCCATGCTGGTACATACGACGGAGAGACGCGAA
ATCTTCATCGTAGAAACGGGCCTCTGTTCATTGGCCGAGCTATCACACTTTACGCAGTTGTT
AGCTCATCCACACCACGAATACCTCAGCGACCTGTACACACCCTGTTCCAGTAGCGGGCGAC
GCGATCACTCGCTCGAACGCCTCACGCGTCTCTTCCCCGATGCCACCGTCCCCGCTACCGTT
CCCGCCGCCCTCTCCATCCTATCTACCATGCAACCAAGCACGCTGGAAACCTTCCCCGACCT
GTTTTGCTTGCCGCTCGGCGAATCCTTCTCCGCGCTGACCGTCTCCGAACACGTCAGTTATA
TCGTAACAAACCAGTACCTGATCAAAGGTATCTCCTACCCTGTCTCCACCACCGTCGTAGGC
CAGAGCCTCATCATCACCCAGACGGACAGTCAAACTAAATGCGAACTGACGCGCAACATGCA
TACCACACACAGCATCACAGTGGCGCTCAACATTTCGCTAGAAAACTGCGCCTTTTGCCAAA
GCGCCCTGCTAGAATACGACGACACGCAAGGCGTCATCAACATCATGTACATGCACGACTCG
GACGACGTCCTTTTCGCCCTGGATCCCTACAACGAAGTGGTGGTCTCATCTCCGCGAACTCA
CTACCTCATGCTTTTGAAAAACGGTACGGTACTAGAAGTAACTGACGTCGTCGTGGACGCCA
CCGACAGTCGTCTCCTCATGATGTCCGTCTACGCGCTATCGGCCATCATCGGCATCTATCTG
CTCTACCGCATGCTCAAGACATGCCGCGCCAAGAGGAGCGGAAGCGGAGCTACTAACTTCAG
CCTGCTGAAGCAGGCTGGAGACGTGGAGGAGAACCCTGGACCTATGTGCCGCCGCCCGGATT
GCGGCTTCTCTTTCTCACCTGGACCGGTGATACTGCTGTGGTGTTGCCTTCTGCTGCCCATT
GTTTCCTCAGCCGCCGTCAGCGTCGCTCCTACCGCCGCCGAGAAAGTCCCCGCGGAGTGCCC
CGAACTAACGCGCCGATGCTTGTTGGGTGAGGTGTTTGAGGGTGACAAGTATGAAAGTTGGC
TGCGCCCGTTGGTGAATGTTACCGGGCGCGATGGCCCGCTATCGCAACTTATCCGTTACCGT
CCCGTTACGCCGGAGGCCGCCAACTCCGTGCTGTTGGACGAGGCTTTCCTGGACACTCTGGC
CCTGCTGTACAACAATCCGGATCAATTGCGGGCCCTGCTGACGCTGTTGAGCTCGGACACAG
CGCCGCGCTGGATGACGGTGATGCGCGGCTACAGCGAGTGCGGCGATGGCTCGCCGGCCGTG
TACACGTGCGTGGACGACCTGTGCCGCGGCTACGACCTCACGCGACTGTCATACGGGCGCAG
CATCTTCACGGAACACGTGTTAGGCTTCGAGCTGGTGCCACCGTCTCTCTTTAACGTGGTGG
TGGCCATACGCAACGAAGCCACGCGTACCAACCGCGCCGTGCGTCTGCCCGTGAGCACCGCT
GCCGCGCCCGAGGGCATCACGCTCTTTTACGGCCTGTACAACGCAGTGAAGGAATTCTGCCT
GCGTCACCAGCTGGACCCGCCGCTGCTACGCCACCTAGATAAATACTACGCCGGACTGCCGC
CCGAGCTGAAGCAGACGCGCGTCAACCTGCCGGCTCACTCGCGCTATGGCCCTCAAGCAGTG
GATGCTCGCTGATAATAGGCTGGAGCCTCGGTGGCCATGCTTCTTGCCCCTTGGGCCTCCCC
CCAGCCCCTCCTCCCCTTCCTGCACCCGTACCCCCGTGGTCTTTGAATAAAGTCTGAGTGGG
CGGC
hCMVgH-2A-gL (ORF-gH-Furin-Linker-P2A-gL) mRNA
(SEQ ID NO: 186)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCCAGGCCUCCCCUCCUACCUCAUCAU
CCUCGCCGUCUGUCUCUUCAGCCACCUACUUUCGUCACGAUAUGGCGCAGAAGCCGUAUCCG
AACCGCUGGACAAAGCGUUUCACCUACUGCUCAACACCUACGGGAGACCCAUCCGCUUCCUG
CGUGAAAAUACCACCCAGUGUACCUACAACAGCAGCCUCCGUAACAGCACGGUCGUCAGGGA
AAACGCCAUCAGUUUCAACUUUUUCCAAAGCUAUAAUCAAUACUAUGUAUUCCAUAUGCCUC
GAUGUCUUUUUGCGGGUCCUCUGGCGGAGCAGUUUCUGAACCAGGUAGAUCUGACCGAAACC
CUGGAAAGAUACCAACAGAGACUUAACACUUACGCGCUGGUAUCCAAAGACCUGGCCAGCUA
CCGAUCUUUUUCGCAGCAGCUAAAGGCACAAGACAGCCUAGGUGAACAGCCCACCACUGUGC
CACCGCCCAUUGACCUGUCAAUACCUCACGUUUGGAUGCCACCGCAAACCACUCCACACGGC
UGGACAGAAUCACAUACCACCUCAGGACUACACCGACCACACUUUAACCAGACCUGUAUCCU
CUUUGAUGGACACGAUCUACUAUUCAGCACCGUCACACCUUGUUUGCACCAAGGCUUUUACC
UCAUCGACGAACUACGUUACGUUAAAAUAACACUGACCGAGGACUUCUUCGUAGUUACGGUG
UCCAUAGACGACGACACACCCAUGCUGCUUAUCUUCGGCCAUCUUCCACGCGUACUUUUCAA
AGCGCCCUAUCAACGCGACAACUUUAUACUACGACAAACUGAAAAACACGAGCUCCUGGUGC
UAGUUAAGAAAGAUCAACUGAACCGUCACUCUUAUCUCAAAGACCCGGACUUUCUUGACGCC
GCACUUGACUUCAACUACCUAGACCUCAGCGCACUACUACGUAACAGCUUUCACCGUUACGC
CGUGGAUGUACUCAAGAGCGGUCGAUGUCAGAUGCUGGACCGCCGCACGGUAGAAAUGGCCU
UCGCCUACGCAUUAGCACUGUUCGCAGCAGCCCGACAAGAAGAGGCCGGCGCCCAAGUCUCC
GUCCCACGGGCCCUAGACCGCCAGGCCGCACUCUUACAAAUACAAGAAUUUAUGAUCACCUG
CCUCUCACAAACACCACCACGCACCACGUUGCUGCUGUAUCCCACGGCCGUGGACCUGGCCA
AACGAGCCCUUUGGACACCGAAUCAGAUCACCGACAUCACCAGCCUCGUACGCCUGGUCUAC
AUACUCUCUAAACAGAAUCAGCAACAUCUCAUCCCCCAAUGGGCACUACGACAGAUCGCCGA
CUUUGCCCUAAAACUACACAAAACGCACCUGGCCUCUUUUCUUUCAGCCUUCGCACGCCAAG
AACUCUACCUCAUGGGCAGCCUCGUCCACUCCAUGCUGGUACAUACGACGGAGAGACGCGAA
AUCUUCAUCGUAGAAACGGGCCUCUGUUCAUUGGCCGAGCUAUCACACUUUACGCAGUUGUU
AGCUCAUCCACACCACGAAUACCUCAGCGACCUGUACACACCCUGUUCCAGUAGCGGGCGAC
GCGAUCACUCGCUCGAACGCCUCACGCGUCUCUUCCCCGAUGCCACCGUCCCCGCUACCGUU
CCCGCCGCCCUCUCCAUCCUAUCUACCAUGCAACCAAGCACGCUGGAAACCUUCCCCGACCU
GUUUUGCUUGCCGCUCGGCGAAUCCUUCUCCGCGCUGACCGUCUCCGAACACGUCAGUUAUA
UCGUAACAAACCAGUACCUGAUCAAAGGUAUCUCCUACCCUGUCUCCACCACCGUCGUAGGC
CAGAGCCUCAUCAUCACCCAGACGGACAGUCAAACUAAAUGCGAACUGACGCGCAACAUGCA
UACCACACACAGCAUCACAGUGGCGCUCAACAUUUCGCUAGAAAACUGCGCCUUUUGCCAAA
GCGCCCUGCUAGAAUACGACGACACGCAAGGCGUCAUCAACAUCAUGUACAUGCACGACUCG
GACGACGUCCUUUUCGCCCUGGAUCCCUACAACGAAGUGGUGGUCUCAUCUCCGCGAACUCA
CUACCUCAUGCUUUUGAAAAACGGUACGGUACUAGAAGUAACUGACGUCGUCGUGGACGCCA
CCGACAGUCGUCUCCUCAUGAUGUCCGUCUACGCGCUAUCGGCCAUCAUCGGCAUCUAUCUG
CUCUACCGCAUGCUCAAGACAUGCCGCGCCAAGAGGAGCGGAAGCGGAGCUACUAACUUCAG
CCUGCUGAAGCAGGCUGGAGACGUGGAGGAGAACCCUGGACCUAUGUGCCGCCGCCCGGAUU
GCGGCUUCUCUUUCUCACCUGGACCGGUGAUACUGCUGUGGUGUUGCCUUCUGCUGCCCAUU
GUUUCCUCAGCCGCCGUCAGCGUCGCUCCUACCGCCGCCGAGAAAGUCCCCGCGGAGUGCCC
CGAACUAACGCGCCGAUGCUUGUUGGGUGAGGUGUUUGAGGGUGACAAGUAUGAAAGUUGGC
UGCGCCCGUUGGUGAAUGUUACCGGGCGCGAUGGCCCGCUAUCGCAACUUAUCCGUUACCGU
CCCGUUACGCCGGAGGCCGCCAACUCCGUGCUGUUGGACGAGGCUUUCCUGGACACUCUGGC
CCUGCUGUACAACAAUCCGGAUCAAUUGCGGGCCCUGCUGACGCUGUUGAGCUCGGACACAG
CGCCGCGCUGGAUGACGGUGAUGCGCGGCUACAGCGAGUGCGGCGAUGGCUCGCCGGCCGUG
UACACGUGCGUGGACGACCUGUGCCGCGGCUACGACCUCACGCGACUGUCAUACGGGCGCAG
CAUCUUCACGGAACACGUGUUAGGCUUCGAGCUGGUGCCACCGUCUCUCUUUAACGUGGUGG
UGGCCAUACGCAACGAAGCCACGCGUACCAACCGCGCCGUGCGUCUGCCCGUGAGCACCGCU
GCCGCGCCCGAGGGCAUCACGCUCUUUUACGGCCUGUACAACGCAGUGAAGGAAUUCUGCCU
GCGUCACCAGCUGGACCCGCCGCUGCUACGCCACCUAGAUAAAUACUACGCCGGACUGCCGC
CCGAGCUGAAGCAGACGCGCGUCAACCUGCCGGCUCACUCGCGCUAUGGCCCUCAAGCAGUG
GAUGCUCGCUGAUAAUAGGCUGGAGCCUCGGUGGCCAUGCUUCUUGCCCCUUGGGCCUCCCC
CCAGCCCCUCCUCCCCUUCCUGCACCCGUACCCCCGUGGUCUUUGAAUAAAGUCUGAGUGGG
CGGC
hCMVUL128-2A-UL131 (ORF-UL128-Furin-Linker-P2A-UL130 Furin-
Linker-F2A-UL131A)
Furin:
(SEQ ID NO: 148)
CCGCGCCAAGAGGAGC
Linker:
GGAAGCGGA
P2A peptide:
(SEQ ID NO: 149)
GCTACTAACTTCAGCCTGCTGAAGCAGGCTGGAGACGTGGAGGAGAACCCTGGACCT
F2A peptide:
(SEQ ID NO: 150)
GTGAAACAGACTTTGAATTTTGACCTTCTCAAGTTGGCGGGAGACGTGGAGTCCAACCCTGG
5′-UTR: bold
3′-UTR: underline
hCMVUL128-2A-UL131 (ORF- UL128- Furin-Linker-P2A-UL130 Furin-
Linker-F2A-UL131A)
(SEQ ID NO: 30)
TCAAGCTTTTGGACCCTCGTACAGAAGCTAATACGACTCACTATAGGGAAATAAGAGAGAAA
AGAAGAGTAAGAAGAAATATAAGAGCCACCATGAGTCCCAAAGATCTGACGCCGTTCTTGAC
GGCGTTGTGGCTGCTATTGGGTCACAGCCGCGTGCCGCGGGTGCGCGCAGAAGAATGTTGCG
AATTCATAAACGTCAACCACCCGCCGGAACGCTGTTACGATTTCAAAATGTGCAATCGCTTC
ACCGTCGCGCTGCGGTGTCCGGACGGCGAAGTCTGCTACAGTCCCGAGAAAACGGCTGAGAT
TCGCGGGATCGTCACCACCATGACCCATTCATTGACACGCCAGGTCGTACACAACAAACTGA
CGAGCTGCAACTACAATCCGTTATACCTCGAAGCTGACGGGCGAATACGCTGCGGCAAAGTA
AACGACAAGGCGCAGTACCTGCTGGGCGCCGCTGGCAGCGTTCCCTATCGATGGATCAATCT
GGAATACGACAAGATAACCCGGATCGTGGGCCTGGATCAGTACCTGGAGAGCGTTAAGAAAC
ACAAACGGCTGGATGTGTGCCGCGCTAAAATGGGCTATATGCTGCAGCGCGCCAAGAGGAGC
GGAAGCGGAGCTACTAACTTCAGCCTGCTGAAGCAGGCTGGAGACGTGGAGGAGAACCCTGG
ACCTATGCTGCGGCTTCTGCTTCGTCACCACTTTCACTGCCTGCTTCTGTGCGCGGTTTGGG
CAACGCCCTGTCTGGCGTCTCCGTGGTCGACGCTAACAGCAAACCAGAATCCGTCCCCGCCA
TGGTCTAAACTGACGTATTCCAAACCGCATGACGCGGCGACGTTTTACTGTCCTTTTCTCTA
TCCCTCGCCCCCACGATCCCCCTTGCAATTCTCGGGGTTCCAGCGGGTATCAACGGGTCCCG
AGTGTCGCAACGAGACCCTGTATCTGCTGTACAACCGGGAAGGCCAGACCTTGGTGGAGAGA
AGCTCCACCTGGGTGAAAAAGGTGATCTGGTACCTGAGCGGTCGGAACCAAACCATCCTCCA
ACGGATGCCCCGAACGGCTTCGAAACCGAGCGACGGAAACGTGCAGATCAGCGTGGAAGACG
CCAAGATTTTTGGAGCGCACATGGTGCCCAAGCAGACCAAGCTGCTACGCTTCGTCGTCAAC
GATGGCACACGTTATCAGATGTGTGTGATGAAGCTGGAGAGCTGGGCTCACGTCTTCCGGGA
CTACAGCGTGTCTTTTCAGGTGCGATTGACGTTCACCGAGGCCAATAACCAGACTTACACCT
TCTGCACCCATCCCAATCTCATCGTTCGCGCCAAGAGGAGCGGAAGCGGAGTGAAACAGACT
TTGAATTTTGACCTTCTCAAGTTGGCGGGAGACGTGGAGTCCAACCCTGGACCTATGCGGCT
GTGTCGGGTGTGGCTGTCTGTTTGTCTGTGCGCCGTGGTGCTGGGTCAGTGCCAGCGGGAAA
CCGCGGAAAAAAACGATTATTACCGAGTACCGCATTACTGGGACGCGTGCTCTCGCGCGCTG
CCCGACCAAACCCGTTACAAGTATGTGGAACAGCTCGTGGACCTCACGTTGAACTACCACTA
CGATGCGAGCCACGGCTTGGACAACTTTGACGTGCTCAAGAGAATCAACGTGACCGAGGTGT
CGTTGCTCATCAGCGACTTTAGACGTCAGAACCGTCGCGGCGGCACCAACAAAAGGACCACG
TTCAACGCCGCCGGTTCGCTGGCGCCACACGCCCGGAGCCTCGAGTTCAGCGTGCGGCTCTT
TGCCAACTGATAATAGGCTGGAGCCTCGGTGGCCATGCTTCTTGCCCCTTGGGCCTCCCCCC
AGCCCCTCCTCCCCTTCCTGCACCCGTACCCCCGTGGTCTTTGAATAAAGTCTGAGTGGGCG
GC
hCMVUL128-2A-UL131 (ORF-UL128-Furin-Linker-P2A-UL130 Furin-
Linker-F2A-UL131A) mRNA
(SEQ ID NO: 187)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGAGUCCCAAAGAUCUGACGCCGUUCUUGAC
GGCGUUGUGGCUGCUAUUGGGUCACAGCCGCGUGCCGCGGGUGCGCGCAGAAGAAUGUUGCG
AAUUCAUAAACGUCAACCACCCGCCGGAACGCUGUUACGAUUUCAAAAUGUGCAAUCGCUUC
ACCGUCGCGCUGCGGUGUCCGGACGGCGAAGUCUGCUACAGUCCCGAGAAAACGGCUGAGAU
UCGCGGGAUCGUCACCACCAUGACCCAUUCAUUGACACGCCAGGUCGUACACAACAAACUGA
CGAGCUGCAACUACAAUCCGUUAUACCUCGAAGCUGACGGGCGAAUACGCUGCGGCAAAGUA
AACGACAAGGCGCAGUACCUGCUGGGCGCCGCUGGCAGCGUUCCCUAUCGAUGGAUCAAUCU
GGAAUACGACAAGAUAACCCGGAUCGUGGGCCUGGAUCAGUACCUGGAGAGCGUUAAGAAAC
ACAAACGGCUGGAUGUGUGCCGCGCUAAAAUGGGCUAUAUGCUGCAGCGCGCCAAGAGGAGC
GGAAGCGGAGCUACUAACUUCAGCCUGCUGAAGCAGGCUGGAGACGUGGAGGAGAACCCUGG
ACCUAUGCUGCGGCUUCUGCUUCGUCACCACUUUCACUGCCUGCUUCUGUGCGCGGUUUGGG
CAACGCCCUGUCUGGCGUCUCCGUGGUCGACGCUAACAGCAAACCAGAAUCCGUCCCCGCCA
UGGUCUAAACUGACGUAUUCCAAACCGCAUGACGCGGCGACGUUUUACUGUCCUUUUCUCUA
UCCCUCGCCCCCACGAUCCCCCUUGCAAUUCUCGGGGUUCCAGCGGGUAUCAACGGGUCCCG
AGUGUCGCAACGAGACCCUUUAUCUGCUGUACAACCGGGAAGGCCAGACCUUGGUGGAGAGA
AGCUCCACCUGGGUGAAAAAGGUGAUCUGGUACCUGAGCGGUCGGAACCAAACCAUCCUCCA
ACGGAUGCCCCGAACGGCUUCGAAACCGAGCGACGGAAACGUGCAGAUCAGCGUGGAAGACG
CCAAGAUUUUUGGAGCGCACAUGGUGCCCAAGCAGACCAAGCUGCUACGCUUCGUCGUCAAC
GAUGGCACACGUUAUCAGAUGUGUGUGAUGAAGCUGGAGAGCUGGGCUCACGUCUUCCGGGA
CUACAGCGUGUCUUUUCAGGUGCGAUUGACGUUCACCGAGGCCAAUAACCAGACUUACACCU
UCUGCACCCAUCCCAAUCUCAUCGUUCGCGCCAAGAGGAGCGGAAGCGGAGUGAAACAGACU
UUGAAUUUUGACCUUCUCAAGUUGGCGGGAGACGUGGAGUCCAACCCUGGACCUAUGCGGCU
GUGUCGGGUGUGGCUGUCUGUUUGUCUGUGCGCCGUGGUGCUGGGUCAGUGCCAGCGGGAAA
CCGCGGAAAAAAACGAUUAUUACCGAGUACCGCAUUACUGGGACGCGUGCUCUCGCGCGCUG
CCCGACCAAACCCGUUACAAGUAUGUGGAACAGCUCGUGGACCUCACGUUGAACUACCACUA
CGAUGCGAGCCACGGCUUGGACAACUUUGACGUGCUCAAGAGAAUCAACGUGACCGAGGUGU
CGUUGCUCAUCAGCGACUUUAGACGUCAGAACCGUCGCGGCGGCACCAACAAAAGGACCACG
UUCAACGCCGCCGGUUCGCUGGCGCCACACGCCCGGAGCCUCGAGUUCAGCGUGCGGCUCUU
UGCCAACUGAUAAUAGGCUGGAGCCUCGGUGGCCAUGCUUCUUGCCCCUUGGGCCUCCCCCC
AGCCCCUCCUCCCCUUCCUGCACCCGUACCCCCGUGGUCUUUGAAUAAAGUCUGAGUGGGCG
GC
hCMVgH-gL-UL128-UL130-UL131A 2a
Furin-
(SEQ ID NO: 148)
CCGCGCCAAGAGGAGC
Linker-GGAAGCGGA
P2A peptide-
(SEQ ID NO: 149)
GCTACTAACTTCAGCCTGCTGAAGCAGGCTGGAGACGTGGAGGAGAACCCTGGACCT
F2A peptide-
(SEQ ID NO: 150)
GTGAAACAGACTTTGAATTTTGACCTTCTCAAGTTGGCGGGAGACGTGGAGTCCAACCCTGG
ACCT
e2a peptide-
(SEQ ID NO: 151)
cagtgtactaattatgctctcttgaaattggctggagatgttgagagcaaccctggacct
t2a-
(SEQ ID NO: 152)
gagggcagaggaagtctgctaacatgcogtgacgtcgaggagaatcctggacct
ORF-gH-Furin-Linker-P2A-gL Furin-Linker-F2A-UL128-Furin-
Linker-e2a-UL130-Furin-Linker-t2a-UL131A
(SEQ ID NO: 31)
TCAAGCTTTTGGACCCTCGTACAGAAGCTAATACGACTCACTATAGGGAAATAAGAGAGAAA
AGAAGAGTAAGAAGAAATATAAGAGCCACCATGCGGCCAGGCCTCCCCTCCTACCTCATCAT
CCTCGCCGTCTGTCTCTTCAGCCACCTACTTTCGTCACGATATGGCGCAGAAGCCGTATCCG
AACCGCTGGACAAAGCGTTTCACCTACTGCTCAACACCTACGGGAGACCCATCCGCTTCCTG
CGTGAAAATACCACCCAGTGTACCTACAACAGCAGCCTCCGTAACAGCACGGTCGTCAGGGA
AAACGCCATCAGTTTCAACTTTTTCCAAAGCTATAATCAATACTATGTATTCCATATGCCTC
GATGTCTTTTTGCGGGTCCTCTGGCGGAGCAGTTTCTGAACCAGGTAGATCTGACCGAAACC
CTGGAAAGATACCAACAGAGACTTAACACTTACGCGCTGGTATCCAAAGACCTGGCCAGCTA
CCGATCTTTTTCGCAGCAGCTAAAGGCACAAGACAGCCTAGGTGAACAGCCCACCACTGTGC
CACCGCCCATTGACCTGTCAATACCTCACGTTTGGATGCCACCGCAAACCACTCCACACGGC
TGGACAGAATCACATACCACCTCAGGACTACACCGACCACACTTTAACCAGACCTGTATCCT
CTTTGATGGACACGATCTACTATTCAGCACCGTCACACCTTGTTTGCACCAAGGCTTTTACC
TCATCGACGAACTACGTTACGTTAAAATAACACTGACCGAGGACTTCTTCGTAGTTACGGTG
TCCATAGACGACGACACACCCATGCTGCTTATCTTCGGCCATCTTCCACGCGTACTTTTCAA
AGCGCCCTATCAACGCGACAACTTTATACTACGACAAACTGAAAAACACGAGCTCCTGGTGC
TAGTTAAGAAAGATCAACTGAACCGTCACTCTTATCTCAAAGACCCGGACTTTCTTGACGCC
GCACTTGACTTCAACTACCTAGACCTCAGCGCACTACTACGTAACAGCTTTCACCGTTACGC
CGTGGATGTACTCAAGAGCGGTCGATGTCAGATGCTGGACCGCCGCACGGTAGAAATGGCCT
TCGCCTACGCATTAGCACTGTTCGCAGCAGCCCGACAAGAAGAGGCCGGCGCCCAAGTCTCC
GTCCCACGGGCCCTAGACCGCCAGGCCGCACTCTTACAAATACAAGAATTTATGATCACCTG
CCTCTCACAAACACCACCACGCACCACGTTGCTGCTGTATCCCACGGCCGTGGACCTGGCCA
AACGAGCCCTTTGGACACCGAATCAGATCACCGACATCACCAGCCTCGTACGCCTGGTCTAC
ATACTCTCTAAACAGAATCAGCAACATCTCATCCCCCAATGGGCACTACGACAGATCGCCGA
CTTTGCCCTAAAACTACACAAAACGCACCTGGCCTCTTTTCTTTCAGCCTTCGCACGCCAAG
AACTCTACCTCATGGGCAGCCTCGTCCACTCCATGCTGGTACATACGACGGAGAGACGCGAA
ATCTTCATCGTAGAAACGGGCCTCTGTTCATTGGCCGAGCTATCACACTTTACGCAGTTGTT
AGCTCATCCACACCACGAATACCTCAGCGACCTGTACACACCCTGTTCCAGTAGCGGGCGAC
GCGATCACTCGCTCGAACGCCTCACGCGTCTCTTCCCCGATGCCACCGTCCCCGCTACCGTT
CCCGCCGCCCTCTCCATCCTATCTACCATGCAACCAAGCACGCTGGAAACCTTCCCCGACCT
GTTTTGCTTGCCGCTCGGCGAATCCTTCTCCGCGCTGACCGTCTCCGAACACGTCAGTTATA
TCGTAACAAACCAGTACCTGATCAAAGGTATCTCCTACCCTGTCTCCACCACCGTCGTAGGC
CAGAGCCTCATCATCACCCAGACGGACAGTCAAACTAAATGCGAACTGACGCGCAACATGCA
TACCACACACAGCATCACAGTGGCGCTCAACATTTCGCTAGAAAACTGCGCCTTTTGCCAAA
GCGCCCTGCTAGAATACGACGACACGCAAGGCGTCATCAACATCATGTACATGCACGACTCG
GACGACGTCCTTTTCGCCCTGGATCCCTACAACGAAGTGGTGGTCTCATCTCCGCGAACTCA
CTACCTCATGCTTTTGAAAAACGGTACGGTACTAGAAGTAACTGACGTCGTCGTGGACGCCA
CCGACAGTCGTCTCCTCATGATGTCCGTCTACGCGCTATCGGCCATCATCGGCATCTATCTG
CTCTACCGCATGCTCAAGACATGCCGCGCCAAGAGGAGCGGAAGCGGAGCTACTAACTTCAG
CCTGCTGAAGCAGGCTGGAGACGTGGAGGAGAACCCTGGACCTATGTGCCGCCGCCCGGATT
GCGGCTTCTCTTTCTCACCTGGACCGGTGATACTGCTGTGGTGTTGCCTTCTGCTGCCCATT
GTTTCCTCAGCCGCCGTCAGCGTCGCTCCTACCGCCGCCGAGAAAGTCCCCGCGGAGTGCCC
CGAACTAACGCGCCGATGCTTGTTGGGTGAGGTGTTTGAGGGTGACAAGTATGAAAGTTGGC
TGCGCCCGTTGGTGAATGTTACCGGGCGCGATGGCCCGCTATCGCAACTTATCCGTTACCGT
CCCGTTACGCCGGAGGCCGCCAACTCCGTGCTGTTGGACGAGGCTTTCCTGGACACTCTGGC
CCTGCTGTACAACAATCCGGATCAATTGCGGGCCCTGCTGACGCTGTTGAGCTCGGACACAG
CGCCGCGCTGGATGACGGTGATGCGCGGCTACAGCGAGTGCGGCGATGGCTCGCCGGCCGTG
TACACGTGCGTGGACGACCTGTGCCGCGGCTACGACCTCACGCGACTGTCATACGGGCGCAG
CATCTTCACGGAACACGTGTTAGGCTTCGAGCTGGTGCCACCGTCTCTCTTTAACGTGGTGG
TGGCCATACGCAACGAAGCCACGCGTACCAACCGCGCCGTGCGTCTGCCCGTGAGCACCGCT
GCCGCGCCCGAGGGCATCACGCTCTTTTACGGCCTGTACAACGCAGTGAAGGAATTCTGCCT
GCGTCACCAGCTGGACCCGCCGCTGCTACGCCACCTAGATAAATACTACGCCGGACTGCCGC
CCGAGCTGAAGCAGACGCGCGTCAACCTGCCGGCTCACTCGCGCTATGGCCCTCAAGCAGTG
GATGCTCGCCGCGCCAAGAGGAGCGGAAGCGGAGTGAAACAGACTTTGAATTTTGACCTTCT
CAAGTTGGCGGGAGACGTGGAGTCCAACCCTGGACCTATGAGTCCCAAAGATCTGACGCCGT
TCTTGACGGCGTTGTGGCTGCTATTGGGTCACAGCCGCGTGCCGCGGGTGCGCGCAGAAGAA
TGTTGCGAATTCATAAACGTCAACCACCCGCCGGAACGCTGTTACGATTTCAAAATGTGCAA
TCGCTTCACCGTCGCGCTGCGGTGTCCGGACGGCGAAGTCTGCTACAGTCCCGAGAAAACGG
CTGAGATTCGCGGGATCGTCACCACCATGACCCATTCATTGACACGCCAGGTCGTACACAAC
AAACTGACGAGCTGCAACTACAATCCGTTATACCTCGAAGCTGACGGGCGAATACGCTGCGG
CAAAGTAAACGACAAGGCGCAGTACCTGCTGGGCGCCGCTGGCAGCGTTCCCTATCGATGGA
TCAATCTGGAATACGACAAGATAACCCGGATCGTGGGCCTGGATCAGTACCTGGAGAGCGTT
AAGAAACACAAACGGCTGGATGTGTGCCGCGCTAAAATGGGCTATATGCTGCAGCGCGCCAA
GAGGAGCGGAAGCGGAcagtgtactaattatgctctcttgaaattggctggagatgttgaga
gcaaccctggacctATGCTGCGGCTTCTGCTTCGTCACCACTTTCACTGCCTGCTTCTGTGC
GCGGTTTGGGCAACGCCCTGTCTGGCGTCTCCGTGGTCGACGCTAACAGCAAACCAGAATCC
GTCCCCGCCATGGTCTAAACTGACGTATTCCAAACCGCATGACGCGGCGACGTTTTACTGTC
CTTTTCTCTATCCCTCGCCCCCACGATCCCCCTTGCAATTCTCGGGGTTCCAGCGGGTATCA
ACGGGTCCCGAGTGTCGCAACGAGACCCTGTATCTGCTGTACAACCGGGAAGGCCAGACCTT
GGTGGAGAGAAGCTCCACCTGGGTGAAAAAGGTGATCTGGTACCTGAGCGGTCGGAACCAAA
CCATCCTCCAACGGATGCCCCGAACGGCTTCGAAACCGAGCGACGGAAACGTGCAGATCAGC
GTGGAAGACGCCAAGATTTTTGGAGCGCACATGGTGCCCAAGCAGACCAAGCTGCTACGCTT
CGTCGTCAACGATGGCACACGTTATCAGATGTGTGTGATGAAGCTGGAGAGCTGGGCTCACG
TeTTCCGGGACTACAGCGTGTCTTTTCAGGTGCGATTGACGTTCACCGAGGCCAATAACCAG
ACTTACACCTTCTGCACCCATCCCAATCTCATCGTTCGCGCCAAGAGGAGCGGAAGCGGAga
gggcagaggaagtctgctaacatgcggtgacgtcgaggagaatcctggacctATGCGGCTGT
GTCGGGTGTGGCTGTCTGTTTGTCTGTGCGCCGTGGTGCTGGGTCAGTGCCAGCGGGAAACC
GCGGAAAAAAACGATTATTACCGAGTACCGCATTACTGGGACGCGTGCTCTCGCGCGCTGCC
CGACCAAACCCGTTACAAGTATGTGGAACAGCTCGTGGACCTCACGTTGAACTACCACTACG
ATGCGAGCCACGGCTTGGACAACTTTGACGTGCTCAAGAGAATCAACGTGACCGAGGTGTCG
TTGCTCATCAGCGACTTTAGACGTCAGAACCGTCGCGGCGGCACCAACAAAAGGACCACGTT
CAACGCCGCCGGTTCGCTGGCGCCACACGCCCGGAGCCTCGAGTTCAGCGTGCGGCTCTTTG
CCAACTGATAATAGGCTGGAGCCTCGGTGGCCATGCTTCTTGCCCCTTGGGCCTCCCCCCAG
CCCCTCCTCCCCTTCCTGCACCCGTACCCCCGTGGTCTTTGAATAAAGTCTGAGTGGGCGGC
ORF-gH-Furin-Linker-P2A-gL Furin-Linker-F2A-UL128-Furin-
Linker-e2a-UL130-Furin-Linker-t2a-UL131A mRNA
(SEQ ID NO: 188)
UCAAGCUUUUGGACCCUCGUACAGAAGCUAAUACGACUCACUAUAGGGAAAUAAGAGAGAAA
AGAAGAGUAAGAAGAAAUAUAAGAGCCACCAUGCGGCCAGGCCUCCCCUCCUACCUCAUCAU
CCUCGCCGUCUGUCUCUUCAGCCACCUACUUUCGUCACGAUAUGGCGCAGAAGCCGUAUCCG
AACCGCUGGACAAAGCGUUUCACCUACUGCUCAACACCUACGGGAGACCCAUCCGCUUCCUG
CGUGAAAAUACCACCCAGUGUACCUACAACAGCAGCCUCCGUAACAGCACGGUCGUCAGGGA
AAACGCCAUCAGUUUCAACUUUUUCCAAAGCUAUAAUCAAUACUAUGUAUUCCAUAUGCCUC
GAUGUCUUUUUGCGGGUCCUCUGGCGGAGCAGUUUCUGAACCAGGUAGAUCUGACCGAAACC
CUGGAAAGAUACCAACAGAGACUUAACACUUACGCGCUGGUAUCCAAAGACCUGGCCAGCUA
CCGAUCUUUUUCGCAGCAGCUAAAGGCACAAGACAGCCUAGGUGAACAGCCCACCACUGUGC
CACCGCCCAUUGACCUGUCAAUACCUCACGUUUGGAUGCCACCGCAAACCACUCCACACGGC
UGGACAGAAUCACAUACCACCUCAGGACUACACCGACCACACUUUAACCAGACCUGUAUCCU
CUUUGAUGGACACGAUCUACUAUUCAGCACCGUCACACCUUGUUUGCACCAAGGCUUUUACC
UCAUCGACGAACUACGUUACGUUAAAAUAACACUGACCGAGGACUUCUUCGUAGUUACGGUG
UCCAUAGACGACGACACACCCAUGCUGCUUAUCUUCGGCCAUCUUCCACGCGUACUUUUCAA
AGCGCCCUAUCAACGCGACAACUUUAUACUACGACAAACUGAAAAACACGAGCUCCUGGUGC
UAGUUAAGAAAGAUCAACUGAACCGUCACUCUUAUCUCAAAGACCCGGACUUUCUUGACGCC
GCACUUGACUUCAACUACCUAGACCUCAGCGCACUACUACGUAACAGCUUUCACCGUUACGC
CGUGGAUGUACUCAAGAGCGGUCGAUGUCAGAUGCUGGACCGCCGCACGGUAGAAAUGGCCU
UCGCCUACGCAUUAGCACUGUUCGCAGCAGCCCGACAAGAAGAGGCCGGCGCCCAAGUCUCC
GUCCCACGGGCCCUAGACCGCCAGGCCGCACUCUUACAAAUACAAGAAUUUAUGAUCACCUG
CCUCUCACAAACACCACCACGCACCACGUUGCUGCUGUAUCCCACGGCCGUGGACCUGGCCA
AACGAGCCCUUUGGACACCGAAUCAGAUCACCGACAUCACCAGCCUCGUACGCCUGGUCUAC
AUACUCUCUAAACAGAAUCAGCAACAUCUCAUCCCCCAAUGGGCACUACGACAGAUCGCCGA
CUUUGCCCUAAAACUACACAAAACGCACCUGGCCUCUUUUCUUUCAGCCUUCGCACGCCAAG
AALUCUACCUCAUGGGCAGCCUCGUCCACUCCAUGCUGGUACAUACGACGGAGAGACGCGAA
AUCUUCAUCGUAGAAACGGGCCUCUGUUCAUUGGCCGAGCUAUCACACUUUACGCAGUUGUU
AGCUCAUCCACACCACGAAUACCUCAGCGACCUGUACACACCCUGUUCCAGUAGCGGGCGAC
GCGAUCACUCGCUCGAACGCCUCACGCGUCUCUUCCCCGAUGCCACCGUCCCCGCUACCGUU
CCCGCCGCCCUCUCCAUCCUAUCUACCAUGCAACCAAGCACGCUGGAAACCUUCCCCGACCU
GUUUUGCUUGCCGCUCGGCGAAUCCUUCUCCGCGCUGACCGUUUCCGAACACGUCAGUUAUA
UCGUAACAAACCAGUACCUGAUCAAAGGUAUCUCCUACCCUGUCUCCACCACCGUCGUAGGC
CAGAGCCUCAUCAUCACCCAGACGGACAGUCAAACUAAAUGCGAACUGACGCGCAACAUGCA
UACCACACACAGCAUCACAGUGGCGCUCAACAUUUCGCUAGAAAACUGCGCCUUUUGCCAAA
GCGCCCUGCUAGAAUACGACGACACGCAAGGCGUCAUCAACAUCAUGUACAUGCACGACUCG
GACGACGUCCUUUUCGCCCUGGAUCCCUACAACGAAGUGGUGGUCUCAUCUCCGCGAACUCA
CUACCUCAUGCUUUUGAAAAACGGUACGGUACUAGAAGUAACUGACGUCGUCGUGGACGCCA
CCGACAGUCGUCUCCUCAUGAUGUCCGUCUACGCGCUAUCGGCCAUCAUCGGCAUCUAUCUG
CUCUACCGCAUGCUCAAGACAUGCCGCGCCAAGAGGAGCGGAAGCGGAGCUACUAACUUCAG
CCUGCUGAAGCAGGCUGGAGACGUGGAGGAGAACCCUGGACCUAUGUGCCGCCGCCCGGAUU
GCGGCUUCUCUUUCUCACCUGGACCGGUGAUACUGCUGUGGUGUUGCCUUCUGCUGCCCAUU
GUUUCCUCAGCCGCCGUCAGCGUCGCUCCUACCGCCGCCGAGAAAGUCCCCGCGGAGUGCCC
CGAACUAACGCGCCGAUGCUUGUUGGGUGAGGUGUUUGAGGGUGACAAGUAUGAAAGUUGGC
UGCGCCCGUUGGUGAAUGUUACCGGGCGCGAUGGCCCGCUAUCGCAACUUAUCCGUUACCGU
CCCGUUACGCCGGAGGCCGCCAACUCCGUGCUGUUGGACGAGGCUUUCCUGGACACUCUGGC
CCUGCUGUACAACAAUCCGGAUCAAUUGCGGGCCCUGCUGACGCUGUUGAGCUCGGACACAG
CGCCGCGCUGGAUGACGGUGAUGCGCGGCUACAGCGAGUGCGGCGAUGGCUCGCCGGCCGUG
UACACGUGCGUGGACGACCUGUGCCGCGGCUACGACCUCACGCGACUGUCAUACGGGCGCAG
CAUCUUCACGGAACACGUGUUAGGCUUCGAGCUGGUGCCACCGUCUCUCUUUAACGUGGUGG
UGGCCAUACGCAACGAAGCCACGCGUACCAACCGCGCCGUGCGUCUGCCCGUGAGCACCGCU
GCCGCGCCCGAGGGCAUCACGCUCUUUUACGGCCUGUACAACGCAGUGAAGGAAUUCUGCCU
GCGUCACCAGCUGGACCCGCCGCUGCUACGCCACCUAGAUAAAUACUACGCCGGACUGCCGC
CCGAGCUGAAGCAGACGCGCGUCAACCUGCCGGCUCACUCGCGCUAUGGCCCUCAAGCAGUG
GAUGCUCGCCGCGCCAAGAGGAGCGGAAGCGGAGUGAAACAGACUUUGAAUUUUGACCUUCU
CAAGUUGGCGGGAGACGUGGAGUCCAACCCUGGACCUAUGAGUCCCAAAGAUCUGACGCCGU
UCUUGACGGCGUUGUGGCUGCUAUUGGGUCACAGCCGCGUGCCGCGGGUGCGCGCAGAAGAA
UGUUGCGAAUUCAUAAACGUCAACCACCCGCCGGAACGCUGUUACGAUUUCAAAAUGUGCAA
UCGCUUCACCGUCGCGCUGCGGUGUCCGGACGGCGAAGUCUGCUACAGUCCCGAGAAAACGG
CUGAGAUUCGCGGGAUCGUCACCACCAUGACCCAUUCAUUGACACGCCAGGUCGUACACAAC
AAACUGACGAGCUGCAACUACAAUCCGUUAUACCUCGAAGCUGACGGGCGAAUACGCUGCGG
CAAAGUAAACGACAAGGCGCAGUACCUGCUGGGCGCCGCUGGCAGCGUUCCCUAUCGAUGGA
UCAAUCUGGAAUACGACAAGAUAACCCGGAUCGUGGGCCUGGAUCAGUACCUGGAGAGCGUU
AAGAAACACAAACGGCUGGAUGUGUGCCGCGCUAAAAUGGGCUAUAUGCUGCAGCGCGCCAA
GAGGAGCGGAAGCGGAcaguguacuaauuaugcucucuugaaauuggcuggagauguugaga
gcaacccuggaccuAUGCUGCGGCUUCUGCUUCGUCACCACUUUCACUGCCUGCUUCUGUGC
GCGGUUUGGGCAACGCCCUGUCUGGCGUCUCCGUGGUCGACGCUAACAGCAAACCAGAAUCC
GUCCCCGCCAUGGUCUAAACUGACGUAUUCCAAACCGCAUGACGCGGCGACGUUUUACUGUC
CUUUUCUCUAUCCCUCGCCCCCACGAUCCCCCUUGCAAUUCUCGGGGUUCCAGCGGGUAUCA
ACGGGUCCCGAGUGUCGCAACGAGACCCUGUAUCUGCUGUACAACCGGGAAGGCCAGACCUU
GGUGGAGAGAAGCUCCACCUGGGUGAAAAAGGUGAUCUGGUACCUGAGCGGUCGGAACCAAA
CCAUCCUCCAACGGAUGCCCCGAACGGCUUCGAAACCGAGCGACGGAAACGUGCAGAUCAGC
GUGGAAGACGCCAAGAUUUUUGGAGCGCACAUGGUGCCCAAGCAGACCAAGCUGCUACGCUU
CGUCGUCAACGAUGGCACACGUUAUCAGAUGUGUGUGAUGAAGCUGGAGAGCUGGGCUCACG
UCUUCCGGGACUACAGCGUGUCUUUUCAGGUGCGAUUGACGUUCACCGAGGCCAAUAACCAG
ACUUACACCUUCUGCACCCAUCCCAAUCUCAUCGUUCGCGCCAAGAGGAGCGGAAGCGGAga
gggcagaggaagucugcuaacaugcggugacgucgaggagaauccuggaccuAUGCGGCUGU
GUCGGGUGUGGCUGUCUGUUUGUCUGUGCGCCGUGGUGCUGGGUCAGUGCCAGCGGGAAACC
GCGGAAAAAAACGAUUAUUACCGAGUACCGCAUUACUGGGACGCGUGCUCUCGCGCGCUGCC
CGACCAAACCCGUUACAAGUAUGUGGAACAGCUCGUGGACCUCACGUUGAACUACCACUACG
AUGCGAGCCACGGCUUGGACAACUUUGACGUGCUCAAGAGAAUCAACGUGACCGAGGUGUCG
UUGCUCAUCAGCGACUUUAGACGUCAGAACCGUCGCGGCGGCACCAACAAAAGGACCACGUU
CAACGCCGCCGGUUCGCUGGCGCCACACGCCCGGAGCCUCGAGUUCAGCGUGCGGCUCUUUG
CCAACUGAUAAUAGGCUGGAGCCUCGGUGGCCAUGCUUCUUGCCCCUUGGGCCUCCCCCCAG
CCCCUCCUCCCCUUCCUGCACCCGUACCCCCGUGGUCUUUGAAUAAAGUCUGAGUGGGCGGC

### Example 18: Immunogenicity Study

The instant study is designed to test the immunogenicity in mice of candidate CMV vaccines comprising an mRNA polynucleotide encoding the gH and gL glycoproteins or the UL128, UL130, and UL131A polypeptides obtained from MCMV.

Mice are vaccinated on week 0 and 4 via intramuscular (IM) or intradermal (ID) routes. One group remains unvaccinated and one is administered inactivated MCMV. Serum is collected from each mouse on weeks 1, 3 (pre-dose) and 5. Individual bleeds are tested for 15 anti-gH and anti-gL activity or anti-UL128, anti-UL130, and anti-UL131A via ELISA assay from all three time points, and pooled samples from week 5 only are tested by Western blot using inactivated MCMV.

### ELISA Immunoassays

Antibody production is measured in a sample by ELISA. Appropriately diluted samples were placed in 96-well plates precoated with a capture antibody directed against an epitope of the antibody. Serum samples typically were diluted 1:100 for the assay. Incubation and washing protocols were performed using routine methods. Data is read at 450 nm with wavelength. Data is reported and plotted.

### Example 19: MCMV Challenge

The instant study is designed to test the efficacy in mice of candidate CMV vaccines against a lethal challenge using a mouse CMV vaccine comprising mRNAs encoding gH and gL or UL128, UL130, and UL131A. Due to the strict species specificity of CMV infection, there is no animal model available for study of HCMV infection and immunity. Murine cytomegalovirus (MCMV) infection is the most widely used mouse model simulating HCMV infection. In the current study, the immunogenicity and protective efficacy of MCMV gH, gL, UL128, UL130, UL131A antigens are investigated.

BALB/c mice are randomly divided into groups. The groups are respectively immunized with (1) 10 μg gB (positive control), (2) 10 μg gH and gL mRNAs (combination of separate sequences), (3) 10 μg gH-gL concatamer mRNA (single sequence), (4) 10 μg UL128, UL130, UL131A mRNAs (combination of separate sequences), (5) 10 μg UL128-UL130-UL131A concatamer mRNA (single sequence), (6) 10 μg gH-gL-UL128-UL130-UL131A concatamer mRNA (single sequence) and (7) PBS. Mice are immunized two times (second dose at day 28) by injection into the right quadriceps muscle (IM) or by intradermal administration (ID), and are challenged with a lethal dose (5×LD50, 200 μl/mouse) of SG-MCMV (Smith strain, 10⁵ PFU) by intraperitoneal injection. This infection causes systemic virus replication in mice and death of all unvaccinated mice within one week after the challenge.

Endpoint is day 5 post infection, death or euthanasia. Animals displaying severe illness as determined by >30% weight loss, extreme lethargy or paralysis are euthanized. The protective effects of the DNA vaccines are evaluated comprehensively using infection symptoms of body temperature, weight loss, and survival. The mice are weighed and assessed daily in order to monitor weight loss, apparent physical condition (bristled hair and wounded skin), body temperature, and behaviour. The mice are humanely euthanized via cervical dislocation after chloroform (inhalation excess) in all cases in order to minimize or avoid animal suffering.

### Example 20: MCMV Neutralization Assay

Mice are immunized according to the methods in Example 18. Mouse serum samples are collected 3 weeks after the second immunization. Serum samples are stored at −20° C. until use. Neutralizing antibody directed against MCMV are determined by a plaque reduction assay, for example, as described in Geoffroy F, et al., Murine cytomegalovirus inactivated by sodium periodate is innocuous and immunogenic in mice and protects them against death and infection. Vaccine. 1996; 14: 1686-1694. Decomplemented sera (30 μl) are serially diluted 2-fold with MEM. Each dilution is mixed with 100 PFU MCMV in 30 μl of MEM and then incubated 1 hour at 4° C. and 1 hour at 37° C. The mixture is layered onto 3T3 monolayers and PFU are calculated by the standard plaque assay. A neutralization titer is expressed as the highest serum dilution required to achieve a 50% reduction in the number of plaques.

### Example 21: IFN-γ ELISPOT Assay

Mice are immunized with gH or gL, or co-immunized with gH/gL mRNAs twice (day 0 and day 28) at a dosage of 10 μg by IM. Two weeks after the second immunization, splenocytes are isolated for ELISPOT assays. Immunospot are coated with rat anti-mouse IFN-γ mAb in accordance with manufacturer instructions, incubated at 4° C. overnight and then blocked with 200 μl of blocking solution. Subsequently, 2×10⁵ lymphocytes are added to the wells in triplicate, stimulated with 10 μg/ml of corresponding gH or gL peptides or a gH/gL polypeptide mixture (for co-immunization group). After 18 hours, the lymphocytes are discarded and biotin-labeled anti-mouse IFN-γ Ab antibody is added to each well and incubated at 37° C. for 1 h. Next, diluted Streptavidin-HRP conjugate solution is added and incubated at room temperature for 2 hours. Finally, the plates are treated with 100 μl of AEC substrate solution and incubated at room temperature for 20 min in the dark. The reaction is stopped by washing with dematerialized water. Spots are quantified by an ELISPOT reader.

TABLE 2
Human Cytomegalovirus Sequences

| SEQ | | | Nucleotide |
|---|---|---|---|
| ID | | | Sequence |
| NO: | Protein Name | Protein Sequence | (SEQ ID NO:) |
| 32 | gi\|52139248\|ref\| | MRPGLPSYLIILAVCLFSHLLSSRYGAEAVSE | 1 |
| | YP_081523.1\| | PLDKAFHLLLNTYGRPIRFLRENTTQCTYNSS | |
| | envelope | LRNSTVVRENAISFNFFQSYNQYYVFHMPRCL | |
| | glycoprotein H | FAGPLAEQFLNQVDLTETLERYQQRLNTYALV | |
| | [Human | SKDLASYRSFSQQLKAQDSLGEQPTTVPPPID | |
| | herpesvirus 5] | LSIPHVWMPPQTTPHGWTESHTTSGLHRPHFN | |
| | | QTCILFDGHDLLFSTVTPCLHQGFYLIDELRY | |
| | | VKITLTEDFFVVTVSIDDDTPMLLIFGHLPRV | |
| | | LFKAPYQRDNFILRQTEKHELLVLVKKDQLNR | |
| | | HSYLKDPDFLDAALDFNYLDLSALLRNSFHRY | |
| | | AVDVLKSGRCQMLDRRTVEMAFAYALALFAAA | |
| | | RQEEAGAQVSVPRALDRQAALLQIQEFMITCL | |
| | | SQTPPRTTLLLYPTAVDLAKRALWTPNQITDI | |
| | | TSLVRLVYILSKQNQQHLIPQWALRQIADFAL | |
| | | KLHKTHLASFLSAFARQELYLMGSIVHSMLVH | |
| | | TTERREIFTVETGLCSLAELSHFTQLLAHPHH | |
| | | EYLSDLYTPCSSSGRRDHSLERLTRLFPDATV | |
| | | PATVPAALSILSTMQPSTLETFPDLFCLPLGE | |
| | | SFSALTVSEHVSYIVTNQYLIKGISYPVSTTV | |
| | | VGQSLIITQTDSQTKCELTRNMHTTHSITVAL | |
| | | NISLENCAFCQSALLEYDDTQGVINIMYMHDS | |
| | | DDVLEALDPYNEVVVSSPRTHYLMLLKNGTVL | |
| | | EVTDVVVDATDSRLLMMSVYALSAUGIYLLYR | |
| | | MLKTC | |
| 33 | gi\|822887470\|gb\| | MPATDTNSTHTTPLHPENQHTLPLHHSTTQPH | 3 |
| | AKI08892.1\| | VQTSDKHADKQHRTQMELDAADYAACAQARQH | |
| | RL1 protein | LYGQTQPQPHAYPNANPQESAHFCTENQHQLT | |
| | [Human | NLLHNIGEGAALGYPVPRAEIRRGGGDWADSA | |
| | herpesvirus 5] | SDFDADCWCMWGRFGTMGRQPVVTLLLARQRD | |
| | | GLADWNVVRCRGTGFRAHDSEDGVSVWRQHLV | |
| | | FLLGGHGRRVQLERPSAGEAQARGLLPRIRIT | |
| | | PISTSPRPKPPQPTTSTASHPHATARPDHTLF | |
| | | PVPSTPSATVHNPRNYAVQLHAETTRTWRWAR | |
| | | RGERGAWMPAETFTCPKDKRPW | |
| 34 | gi\|822888315\|gb\| | MPATDTNSTHTTPLHPEDQHTLPLQHNTTQPH | 6 |
| | AKI09732.1\| | VQTSDKPADKQHRTQMELDAADYAACAQARQH | |
| | RL1 protein | LYGQTQPQLHAYPNANPQESAHFCTDNQHRLT | |
| | [Human | NLLHNIGEGAALGYPVPRAEIRRGGGDWADSA | |
| | herpesvirus 5] | SDFDADCWCMWGRFGTMGRQPVVTLLLARQRD | |
| | | GLADWNVVRCRGTGFRAHDSEDGVSVWRQHLV | |
| | | FLLGGHGRRVQLERPSAGEAQARGLLPRIRIT | |
| | | PVSTSPRPKAPQPTTSTASHPHATARPDHTLF | |
| | | PVPSTPSATVHNPRNYAVQLHAETTRTWRWAR | |
| | | RGERGAWMPAETFTCPKDKRPW | |
| 35 | gi\|136968lsp\| | MGRKEMMVRDVPKMVFLISISFLLVSFINCKV | |
| | P16750.1\|GO_HCMVA | MSKALYNRPWRGLVLSKIGKYKLDQLKLEILR | |
| | RecName: | QLETTISTKYNVSKQPVKNLTMNMTEFPQYYI | |
| | Full = Glyco- | LAGPIQNYSITYLWFDFYSTQLRKPAKYVYSQ | |
| | protein O; | YNHTAKT1TFRPPPCGTVPSMTCLSEMLNVSK | |
| | Short = gO; | RNDTGEQGCGNFTTFNPMFFNVPRWNTKLYVG | |
| | Flags: Precursor | PTKVNVDSQTIYFLGLTALLLRYAQRNCTHSF | |
| | | YLVNAMSRNLFRVPKYINGTKLKNTMRKLKRK | |
| | | QAPVKEQFEKKAKKTQSTTTPYFSYTTSAALN | |
| | | VTTNVTYSITTAARRVSTSTIAYRPDSSFMKS | |
| | | IMATQLRDLATWVYTTLRYRQNPFCEPSRNRT | |
| | | AVSEFMKNTHVLIRNETPYTIYGTLDMSSLYY | |
| | | NETMFVENKTASDSNKTTPTSPSMGFQRTFID | |
| | | PLWDYLDSLLFLDEIRNFSLRSPTYVNLTPPE | |
| | | HRRAVNLSTLNSLWWWLQ | |
| 36 | gi\|583844649\|gb\| | MECNTLVLGLLVLSVVASSNNTSTASTPRPSS | |
| | AHI58989.1\| | STHASTTVKATTVATTSTTTATSTSSTTSAKP | |
| | envelope | GFTTHDPNVMRPHAHNDFYNAHCTSHMYELSL | |
| | glycoprotein N | SSFAAWWTMLNALILMGAFCIVLRHCCFQNFT | |
| | [Human | ATTTKGY | |
| | herpesvirus 5] | | |
| 37 | gi\|136994\|sp\| | MAPSHVDKVNTRTWSASIVFMVLTFVNVSVHL | |
| | P16733.1\|GM_HCMVA | VLSNFPHLGYPCVYYHVVDFERLNMSAYNVMH | |
| | RecName: | LHTPMLFLDSVQLVCYAVFMQIVFLAVTIYYL | |
| | Full = Envelope | VCWIKISMRKDKGMSLNQSTRDISYMGDSLTA | |
| | glycoprotein M; | FLFILSMDTFQLFTLTMSFRLPSMIAFMAAVH | |
| | Short = gM | FFCLTIFNVSMVTQYRSYKRSLFFFSRLHPKL | |
| | | KGTVQFRTLIVNLVEVALGFNTTVVAMALCYG | |
| | | FGNNFFVRTGHMVLAVFVVYAIISIIYFLLIE | |
| | | AVFFQYVKVQFGYHLGAFFGLCGLIYPIVQYD | |
| | | TFLSNEYRTGISWSFGMLFFIWAMFTTCRAVR | |
| | | YFRGRGSGSVKYQALATASGEEVAVLSHHDSL | |
| | | ESRRLREEEDDDDDEDFEDA | |
| 38 | gi\|77455773\|gb\| | MSPKDLTPFLTALWLLLGHSRVLRVRAEECCE | 13 |
| | ABA86616.1\| | FINVNHPPERCYDFKMCNRFIVALRCPDGEVC | |
| | UL128 [Human | YSPEKTAEIRGIVTTMTHSLTRQVVHNKLTSC | |
| | herpesvirus 5] | NYNPLYLEADGRIRCGKVNDKAQYLLGAAGSV | |
| | | PYRWINLEYDKITRIVGLDQYLESVKKHKRLD | |
| | | VCRAKMGYMLQ | |
| 39 | gi\|77455773\|gb\| | MSPKDLTPFLTALWLLLGHSRVLRVRAEECCE | 14 |
| | ABA86616.1\| | FINVNHPPERCYDFKMCNRFTVALRCPDGEVC | |
| | UL128 [Human | YSPEKTAEIRGIVTTMTHSLTRQVVHNKLTSC | |
| | herpesvirus 5] | NYNPLYLEADGRIRCGKVNDKAQYLLGAAGSV | |
| | | PYRWINLEYDKITRIVGLDQYLESVKKHKRLD | |
| | | VCRAKMGYMLQ | |
| 40 | gi\|822891002\|gb\| | MPATDTNSTHTTPLHPEHHHSTTQPHAQTSDK | 15 |
| | AKI12403.1\| | HADKQHRTQMELDAADYAACAQARQHLYGQTQ | |
| | RL1 protein | PQLHAYPNANPQESAHFCTENQHQLTNLLHNI | |
| | [Human | GEGAALGYPVPRAEIRRGGGDWADSASDFDAD | |
| | herpesvirus 5] | CWCMWGRFGTMGRQPVVTLLLARQRDGLADWN | |
| | | VVRCRGTGFRAHDSEDGVSVWRQHLVFLLGGH | |
| | | GRRVQLERPSAGEAQARGLLPRIRITPISTSP | |
| | | RPKPPQPTTSTASHPHATARPDHTLFPVPSTP | |
| | | SATVHNPRNYAVQLHAETTRTWRWARRGERGA | |
| | | WMPAETFTCPKDKRPW | |
| 41 | gi\|52139182\|ref\| | MPATDTNSTHTTPLHPEDQHTLPLHHSTTQPH | 16 |
| | YP_081455.1\| | VQTSDKHADKQHRTQMELDAADYAACAQARQH | |
| | protein RL1 | LYGQTQPQLHAYPNANPQESAHFRTENQHQLT | |
| | [Human | NLLHNIGEGAALGYPVPRAEIRRGGGDWADSA | |
| | herpesvirus 5] | SDFDADCWCMWGRFGTMGRQPVVTLLLARQRD | |
| | | GLADWNVVRCRGTGFRAHDSEDGVSVWRQHLV | |
| | | FLLGGHGRRVQLERPSAGEAQARGLLPRIRIT | |
| | | PISTSPRPKPPQPTTSTASHPHATARPDHTLF | |
| | | PVPSTPSATVHNPRNYAVQLHAETTRTWRWAR | |
| | | RGERGAWMPAETFTCPKDKRPW | |
| 42 | gi\|52139291\|ref\| | MRLCRVWLSVCLCAVVLGQCQRETAEKNDYYR | 17 |
| | YP_081566.1\| | VPHYWDACSRALPDQTRYKYVEQLVDLTLNYH | |
| | envelope protein | YDASHGLDNFDVLKRINVTEVSLLISDFRRQN | |
| | UL131A | RRGGTNKRTIFNAAGSLAPHARSLEFSVRLFA | |
| | [Human | N | |
| | herpesvirus 5] | | |
| 43 | gi\|5213929l\|ref\| | MRLCRVWLSVCLCAVVLGQCQRETAEKNDYYR | 18 |
| | YP_081566.1\| | VPHYWDACSRALPDQTRYKYVEQLVDLTLNYH | |
| | envelope protein | YDASHGLDNFDVLKRINVTEVSLLISDFRRQN | |
| | UL131A | RRGGTNKRTTFNAAGSLAPHARSLEFSVRLFA | |
| | [Human | N | |
| | herpesvirus 5] | | |
| 44 | gi\|52139182\|ref\| | MPATDTNSTHTTPLHPEDQHTLPLHHSTTQPH | 2 |
| | YP_081455.1\| | VQTSDKHADKQHRTQMELDAADYAACAQARQH | |
| | protein RL1 | LYGQTQPQLHAYPNANPQESAHFRTENQHQLT | |
| | [Human | NLLHNIGEGAALGYPVPRAEIRRGGGDWADSA | |
| | herpesvirus 5] | SDFDADCWCMWGRFGTMGRQPVVTLLLARQRD | |
| | | GLADWNVVRCRGTGFRAHDSEDGVSVWRQHLV | |
| | | FLLGGHGRRVQLERPSAGEAQARGLLPRIRIT | |
| | | PISTSPRPKPPQPTTSTASHPHATARPDHTLF | |
| | | PVPSTPSATVHNPRNYAVQLHAETTRTWRWAR | |
| | | RGERGAWMPAETFTCPKDKRPW | |
| 45 | gi\|52139182\|ref\| | MPATDTNSTHTTPLHPEDQHTLPLHHSTTQPH | 4 |
| | YP_081455.1\| | VQTSDKHADKQHRTQMELDAADYAACAQARQH | |
| | protein RL1 | LYGQTQPQLHAYPNANPQESAHFRTENQHQLT | |
| | [Human | NLLHNIGEGAALGYPVPRAEIRRGGGDWADSA | |
| | herpesvirus 5] | SDFDADCWCMWGRFGTMGRQPVVTLLLARQRD | |
| | | GLADWNVVRCRGTGFRAHDSEDGVSVWRQHLV | |
| | | FLLGGHGRRVQLERPSAGEAQARGLLPRIRIT | |
| | | PISTSPRPKPPQPTTSTASHPHATARPDHTLF | |
| | | PVPSTPSATVHNPRNYAVQLHAETTRTWRWAR | |
| | | RGERGAWMPAETFTCPKDKRPW | |
| 46 | gi\|822888315\|gb\| | MPATDTNSTHTTPLHPEDQHTLPLQHNTTQPH | 6 |
| | AKI09732.1\| | VQTSDKPADKQHRTQMELDAADYAACAQARQH | |
| | RL1 protein | LYGQTQPQLHAYPNANPQESAHFCTDNQHRLT | |
| | [Human | NLLHNIGEGAALGYPVPRAEIRRGGGDWADSA | |
| | herpesvirus 5] | SDFDADCWCMWGRFGTMGRQPVVTLLLARQRD | |
| | | GLADWNVVRCRGTGFRAHDSEDGVSVWRQHLV | |
| | | FLLGGHGRRVQLERPSAGEAQARGLLPRIRIT | |
| | | PVSTSPRPKAPQPTTSTASHPHATARPDHTLF | |
| | | PVPSTPSATVHNPRNYAVQLHAETTRTWRWAR | |
| | | RGERGAWMPAETFTCPKDKRPW | |
| 47 | gi\|52139182\|ref\| | MPATDTNSTHTTPLHPEDQHTLPLHHSTTQPH | 7 |
| | YP_081455.1\| | VQTSDKHADKQHRTQMELDAADYAACAQARQH | |
| | protein RL1 | LYGQTQPQLHAYPNANPQESAHFRTENQHQLT | |
| | [Human | NLLHNIGEGAALGYPVPRAEIRRGGGDWADSA | |
| | herpesvirus 5] | SDFDADCWCMWGRFGTMGRQPVVTLLLARQRD | |
| | | GLADWNVVRCRGTGFRAHDSEDGVSVWRQHLV | |
| | | FLLGGHGRRVQLERPSAGEAQARGLLPRIRIT | |
| | | PISTSPRPKPPQPTTSTASHPHATARPDHTLF | |
| | | PVPSTPSATVHNPRNYAVQLHAETTRTWRWAR | |
| | | RGERGAWMPAETFTCPKDKRPW | |
| 48 | hCMV- | SSFWTLVQKLIRLTIGK-ERKEE-EEI-EPPC | 8 |
| | gHtruncFLAG, | GQASPPTSSSSPSVSSATYFRHDMAQKPYPNR | |
| | glycoprotein H | WTKRFTYCSTPTGDPSASCVKIPPSVPTTAAS | |
| | Ectodomain | VTARSSGKTPSVSTFSKAIINTMYSICLDVFL | |
| | | RVLWRSSF-TR-I-PKPWKDTNRDLTITRWYP | |
| | | KTWPATDLFRSS-RHKTA-VNSPPLCHRPLTC | |
| | | QYLTFGCHRKPLHTAGQNHIPPQDYTDHTLTR | |
| | | PVSSLMDTIYYSAPSHLVCTKAFTSSTNYVTL | |
| | | K-H-PRTSS-LRCPTTTHPCCLSSAIFHAYFS | |
| | | KRPINATTLYYDKLKNTSSWC-LRKIN-VTLI | |
| | | SKTRTFLTPHLTSTT-TSAHYYVTAFTVTPWM | |
| | | YSRAVDVRCWTAAR-KWPSPTH-HCSQQPDKK | |
| | | RPAPKSPSHGPTARPHSYKYKNL-SPASHKHH | |
| | | HAPRCCCIPRPWTWPNEPFGHRIRSPTSPASY | |
| | | AWSTYSLNRISNISSPNGHYDRSPTLP-NYTK | |
| | | RTWPLFFQPSHAKNSTSWAASSTPCWYIRRRD | |
| | | AKSSS-KRASVHWPSYHTLRSC-LIHTTNTSA | |
| | | TCTHPVPVAGDAITRSNASRVSSPMPPSPLPF | |
| | | PPPSPSYLPCNQARWKPSPTCFACRSANPSPR | |
| | | -PSPNTSVIS-QTST-SKVSPTLSPPPS-ARA | |
| | | SSSPRRTVKLNAN-RATCIPHTASQWRSTFR- | |
| | | KTAPFAKAPC-NTTTRKASSTSCTCTTRTTSF | |
| | | SPWIPTTKWWSHLRELTTSCF-KTVRY-K-LT | |
| | | SSWTPPTITRTMTISDDNRLEPRWPCFLPLGP | |
| | | PPSPSSPSCTRTPVVFE-SLSGR | |
| 49 | hCMVgHtrunc6XHis, | SSFWTLVQKLIRLTIGK-ERKEE-EEI-EPPC | 9 |
| | glycoprotein H | GQASPPTSSSSPSVSSATYFRHDMAQKPYPNR | |
| | Ectodomain- | WTKRFTYCSTPTGDPSASCVKIPPSVPTTAAS | |
| | 6XHis tag | VTARSSGKTPSVSTFSKAIINTMYSICLDVFL | |
| | | RVLWRSSF-TR-I-PKPWKDTNRDLTLTRWYP | |
| | | KTWPATDLFRSSRHKTA-VNSPPLCHRPLTCQ | |
| | | YLTFGCHRKPLHTAGQNHIPPQDYTDHTLTRP | |
| | | VSSLMDTIYYSAPSHLVCTKAFTSSTNYVTLK | |
| | | -H-PRTSS-LRCP-TTTHPCCLSSAIFHAYFS | |
| | | KRPINATTLYYDKLKNTSSWC-LRKIN-TVTL | |
| | | ISKTRTFLTPHLTSTT-TSAHYYVTAFTVTPW | |
| | | MYSRAVDVRCWTAAR-KWPSPTH-HCSQQPDK | |
| | | KRPAPKSPSHGP-TARPHSYKYKNL-SPASHK | |
| | | HHHAPRCCCIPRPWTWPNEPFGHRIRSPTSPA | |
| | | SYAWSTYSLNRISNISSPNGHYDRSPTLP-NY | |
| | | TKRTWPLFFQPSHAKNSTSWAASSTPCWYIRR | |
| | | RDAKSSS-KRASVHWPSYHTLRSC-LIHTTNT | |
| | | SATCTHPVPVAGDAITRSNASRVSSPMPPSPL | |
| | | PFPPPSPSYLPCNQARWKPSPTCFACRSANPS | |
| | | PR-PSPNTSVIS-QTST-SKVSPTLSPPPS-A | |
| | | RASSSPRRTVKLNAN-RATCIPHTASQWRSTF | |
| | | R-KTAPFAKAPC-NTTTRKASSTSCTCTTRTT | |
| | | SFSPWIPTTKWWSHLRELTTSCF-KTVRY-K- | |
| | | LTSSWTPPTTITTITDDNRLEPRWPCFIPLGP | |
| | | PPSPSSPSCTRTPVVFE-SLSGR | |
| 50 | hCMV_TrgB, | SSFWTLVQKLIRLTIGK-ERKEE-EEI-EPPW | 10 |
| | glycoprotein B | NPGSGAW-ALTCVSSVWVLRFPHLLLVELLLL | |
| | (ectodomain) | TVTIPLIRRLLLTLDPVQSLNA-LLPKRSAMV | |
| | | LTRPSTTLPSSTEMWWGSIPPSTPIACVLWPR | |
| | | VRILFALNVISSAPR-SPSMKTWTRASWWSTN | |
| | | ATSSRTPLRYESTRRF-RFVVATLTSTPLICW | |
| | | AATRNTWRLLCGRFIISTATVSATVPTAAL-Q | |
| | | ARFSWLIIGTAMKTKPCN-CPTIIPTPTVPVT | |
| | | -RSRINGTAAAAPGSIVRPVI-IVW-PSLLRA | |
| | | PNILIIFSPLPRVTWLTFLLSTTEPIAMPATL | |
| | | EKTPTSFSFFRTTLSSPTLEDR1LR-RPTGWW | |
| | | LFLNVRTR-SPGIYRTKRMSLVNSLSGKPRNA | |
| | | PFVPKPRTRITFLLPK-PPLSYLRSKR-TCPT | |
| | | LRWTAYVMRLISYSRFSILHTIKHMKNMETCP | |
| | | SLKPLVVW-CSGKVSSKNLWWNSNVWPTAPV- | |
| | | ILLIIEPKEVQMATMQLIYPTWNRCTIWSTPS | |
| | | CSSPMTRCAVTSTGRWRKSQKPGVWINGAP-R | |
| | | SSRNSARSTRQPFSRPFTTNRLPRVSWVMSWA | |
| | | WPAA-PSTKPASRCCVI-T-RSRQDAATHDPW | |
| | | SSLISPTARTCSTVNWARTTKSCWATTALRNV | |
| | | SFPASRSSSPGTRPTSTWTTSSNA-LTSAVSP | |
| | | PSTA-SPWISTRWKIPTSGYWNFTRRKSCVPA | |
| | | TFLTSKRSCANSTRTSSDNRLEPRWPCFLPLG | |
| | | PPPSPSSPSCTRTPVVFE-SLSGR | |
| 51 | hCMV_TrgBFLAG, | SSFWTLVQKLIRLTIGK-ERKEE-EEIEPPWN | 11 |
| | hCMV | PGSGAW-SALTCVSSVWVLRFPHLLLVELLLL | |
| | glycoproteinB | TVTIPLIRRLLLTLDPVQSLNA-LLPKRSAMV | |
| | ectodomain- | LTRPSTTLPSSTEMWWGSIPPSTPIACVLWPR | |
| | FLAG | VRILFALNVISSAPR-SPSMKTWTRASWWSTN | |
| | | ATSSRTPLRYESTRRF-RFVVATLTSTPLICW | |
| | | AATRNTWRLLCGRFIISTATVSATVPTAAL-Q | |
| | | ARFSWLIIGTAMKTKPCN-CPTIIPTPTVPVT | |
| | | -RSRINGTAAAAPGSIVRPVI-IVW-PSLLRA | |
| | | PNILIIFSPLPRVTWLTFLLSTTEPIAMPATL | |
| | | EKTPTSFSFFRTTLSSPTLEDRILR-RPTGWW | |
| | | LFLNVRTR-SPGIYRTKRMSLVNSLSGKPRNA | |
| | | PFVPKPRTRITFLLPK-PPLSYLRSKR-TCPT | |
| | | LRWTAYVMRL-ISYSRFSIIHTIKHMKNMETC | |
| | | PSLKPLVVW-CSGKVSSKNLWWNSNVWPTAPV | |
| | | -ILLIIEPKEVQMATMQLIYPTWNRCTIWSTP | |
| | | SCSSPMTRCAVTSTGRWRKSQKPGVWINGAP- | |
| | | RSSRNSARSTRQPFSRPFTTNRLPRVSWVMSW | |
| | | AWPAA-PSTKPASRCCVI-T-RSRQDAATHDP | |
| | | WSSLISPTARTCSTVNWARTTKSCWATTALRN | |
| | | VSFPASRSSSPGTRPTSTWTTSSNA-LTSAVS | |
| | | PPSTA-SPWISTRWKIPTSGYWNFTRRKSCVP | |
| | | ATFLTSKRSCANSTRTSRITRTMTISDNRLEP | |
| | | RWPCFLPLGPPPSPSSPSCTRTPVVFE-SLSG | |
| | | R | |
| 52 | hCMV- | SSFWTLVQKLIRLTIGK-ERKEE-EEIEPPWN | 12 |
| | TrgBXHis, | PGSGAW-SALTCVSSVWVLRFPHLLLVELLLL | |
| | hCMV | TVTIPLIRRLLLTLDPVQSLNA-LLPKRSAMV | |
| | glycoprotein | LTRPSTTLPSSTEMWWGSIPPSTPIACVLWPR | |
| | ectodomain- | VRILFALNVISSAPR-SPSMKTWTRASWWSTN | |
| | 6XHis tag | ATSSRTPLRYESTRRF-RFVVATLTSTPLICW | |
| | | AATRNTWRLLCGRPUSTATVSATVPTAAL-QA | |
| | | RFSWLIIGTAMKTKPCN-CPTIIPTPTVPVT- | |
| | | RSRINGTAAAAPGSIVRPVI-IVW-PSLLRAP | |
| | | NILIIFSPLPRVTWLTFLLSTTEPIAMPATLE | |
| | | KTPTSFSFFRTTLSSPTLEDRILR-RPTGWWL | |
| | | FLNVRTR-SPGIYRTKRMSLVNSLSGKPRNAP | |
| | | FVPKPRTRITFLLPK-PPLSYLRSKR-TCPTL | |
| | | RWTAYVMRL-ISYSRFSILHTIKHMKNMETCP | |
| | | SLKPLVVW-CSGKVSSKNLWWNSNVWPTAPV- | |
| | | ILLIIEPKEVQMATMQLIYPTWNRCTIWSTPS | |
| | | CSSPMTRCAVTSTGRWRKSQKPGVWINGAP-R | |
| | | SSRNSARSTRQPFSRPFTTNRLPRVSWVMSWA | |
| | | WPAA-PSTKPASRCCVI-T-RSRQDAATHDPW | |
| | | SSLISPTARTCSTVNWARTTKSCWATTALRNV | |
| | | SFPASRSSSPGTRPTSTWTTSSNA-LTSAVSP | |
| | | PSTA-SPWISTRWKIPTSGYWNFTRRKSCVPA | |
| | | TFLTSKRSCANSTRTSSTITTITDNRLEPRWP | |
| | | CFLPLGPPPSPSSPSCTRTPVVFE-SLSGR | |
| 55 | hCMV | MCRRPDCGFSFSPGPVILLWCCLLLPIVSSAA | 3 |
| | glycoprotein L | VSVAPTAAEKVPAECPELTRRCLLGEVFEGDK | |
| | | YESWLRPLVNVTGRDGPLSQLIRYRPVTPEAA | |
| | | NSVLLDEAFLDTLALLYNNPDQLRALLTLLSS | |
| | | DTAPRWMTVMRGYSECGDGSPAVYTCVDDLCR | |
| | | GYDLTRLSYGRSIFTEHVLGFELVPPSIFNVV | |
| | | VAIRNEATRTNRAVRLPVSTAAAPEGITLFYG | |
| | | LYNAVKEFCLRHQLDPPLLRHLDKYYAGLPPE | |
| | | LKQTRVNIPAHSRYGPQAVDAR | |
| 56 | hCMV | MESRIWCLVVCVNLCIVCLGAAVSSSSTRGTS | 5 |
| | glycoprotein B | ATHSHHSSHTTSAAHSRSGSVSQRVTSSQTVS | |
| | | HGVNETIYNTTLKYGDVVGVNTTKYPYRVCSM | |
| | | AQGTDLIRFERNIVCTSMKPINEDLDEGIMVV | |
| | | YKRNIVAHTFKVRVYQKVITFRRSYAYIHTTY | |
| | | LLGSNTEYVAPPMWEIHHINSHSQCYSSYSRV | |
| | | IAGTVFVAYHRDSYENKTMQLMPDDYSNTHST | |
| | | RYVTVKDQWHSRGSTWLYRETCNLNCMVTITT | |
| | | ARSKYPYHFFATSTGDVVDISPFYNGTNRNAS | |
| | | YFGENADKFFIFPNYTIVSDFGRPNSALETHR | |
| | | LVAFLERADSVISWDIQDEKNVTCQLTFWEAS | |
| | | ERTIRSEAEDSYHFSSAKMTATFLSKKQEVNM | |
| | | SDSALDCVRDEAINKLQQIFNTSYNQTYEKYG | |
| | | NVSVFETTGGLVVFWQGIKQKSLVELERLANR | |
| | | SSLNLTHNRTKRSTDGNNATHLSNMESVHNLV | |
| | | YAQLQFTYDTLRGYINRALAQIAEAWCVDQRR | |
| | | TLEVFKELSKINPSAILSAIYNKPIAARFMGD | |
| | | VLGLASCVTINQTSVKVLRDMNVKESPGRCYS | |
| | | RPVVIFNFANSSYVQYGQLGEDNEILLGNHRT | |
| | | EECQLPSLKIFIAGNSAYEYVDYLFKRMIDLS | |
| | | SISTVDSMIALDIDPLENTDFRVLELYSQKEL | |
| | | RSSNVFDLEEIMREFNSYKQRVKYVEDKVVDP | |
| | | LPPYLKGLDDLMSGLGAAGKAVGVAIGAVGGA | |
| | | VASVVEGVATFLKNPFGAFTIILVAIAVVIIT | |
| | | YLIYTRQRRLCTQPLQNLFPYLVSADGTTVTS | |
| | | GSTKDTSLQAPPSYEESVYNSGRKGPGPPSSD | |
| | | ASTAAPPYTNEQAYQMLLALARLDAEQRAQQN | |
| | | GTDSLDGRTGTQDKGQKPNILDRLRHRKNGYR | |
| | | HLKDSDEEENV | |
| 57 | hCMV UL130 | MLRLLLRHHFHCLLLCAVWATPCLASPWSTLT | 15 |
| | | ANQNPSPPWSKLTYSKPHDAATFYCPFLYPSP | |
| | | PRSPLQFSGFQRVSTGPECRNETLYLLYNREG | |
| | | QTLVERSSTWVKKVIWYLSGRNQTILQRMPRT | |
| | | ASKPSDGNVQISVEDAKIFGAHMVPKQTKLLR | |
| | | FVVNDGTRYQMCVMKLESWAHVFRDYSVSFQV | |
| | | RLTFTEANNQTYTFCTHPNLIV | |
| 53 | Ig heavy chain | MDWTWILFLVAAATRVHS | |
| | epsilon-1 signal | | |
| | peptide (IgE | | |
| | HCSP) | | |
| 54 | IgGk chain V-III | METPAQLLFLLLLWLPDTTG | |
| | region HAH | | |
| | signal peptide | | |
| | (IgGk SP) | | |

- Represents a stop sequence

TABLE 3
CMV (Human herpesvirus 5) Amino Acid Sequences

| | | GenBank |
|---|---|---|
| Protein | Name | Accession |
| Glycoprotein H | envelope glycoprotein H [Human herpesvirus 5] | ACZ79986.1 |
| Glycoprotein H | envelope glycoprotein H [Human herpesvirus 5] | AAA45918.1 |
| Glycoprotein H | envelope glycoprotein H [Human herpesvirus 5] | ACS93310.2 |
| Glycoprotein H | envelope glycoprotein H [Human herpesvirus 5] | AAA45911.1 |
| Glycoprotein H | envelope glycoprotein H [Human herpesvirus 5] | AAA98521.1 |
| Glycoprotein H | glycoprotein H [Human herpesvirus 5] | BAF44184.1 |
| Glycoprotein H | envelope glycoprotein H [Human herpesvirus 5] | AAA45912.1 |
| Glycoprotein H | envelope glycoprotein H [Human herpesvirus 5] | AKI21335.1 |
| Glycoprotein H | envelope glycoprotein H [Human herpesvirus 5] | AIC80661.1 |
| Glycoprotein H | envelope glycoprotein H [Human herpesvirus 5] | AKI11476.1 |
| Glycoprotein H | envelope glycoprotein H [Human herpesvirus 5] | ACZ80151.1 |
| Glycoprotein H | envelope glycoprotein H [Human herpesvirus 5] | AHV84023.1 |
| Glycoprotein H | envelope glycoprotein H [Human herpesvirus 5] | AAA45917.1 |
| Glycoprotein H | envelope glycoprotein H [Human herpesvirus 5] | AAA45915.1 |
| Glycoprotein H | envelope glycoprotein H [Human herpesvirus 5] | AFR55394.1 |
| Glycoprotein H | envelope glycoprotein H [Human herpesvirus 5] | AKI14309.1 |
| Glycoprotein H | envelope glycoprotein H [Human herpesvirus 5] | AKI11640.1 |
| Glycoprotein H | glycoprotein H [Human herpesvirus 5] | BAF44187.1 |
| Glycoprotein H | envelope glycoprotein H [Human herpesvirus 5] | AKI18318.1 |
| Glycoprotein H | envelope glycoprotein H [Human herpesvirus 5] | AHB20043.1 |
| Glycoprotein H | envelope glycoprotein H [Human herpesvirus 5] | AAA45909.1 |
| Glycoprotein H | glycoprotein H [Human herpesvirus 5] | BAF44190.1 |
| Glycoprotein H | RecName: Full = Envelope glycoprotein H; | P12824.1 |
| | Short = gH; Flags: Precursor | |
| Glycoprotein H | envelope glycoprotein H [Human herpesvirus 5] | AKI07789.1 |
| Glycoprotein H | glycoprotein H [Human herpesvirus 5] | BAF44183.1 |
| Glycoprotein H | envelope glycoprotein H [Human herpesvirus 5] | AGL96664.1 |
| Glycoprotein H | glycoprotein H [Human herpesvirus 5] | BAF44189.1 |
| Glycoprotein H | envelope glycoprotein H [Human herpesvirus 5] | AKI08793.1 |
| Glycoprotein H | glycoprotein H [Human herpesvirus 5] | BAF44185.1 |
| Glycoprotein H | envelope glycoprotein H [Human herpesvirus 5] | ADV04392.1 |
| Glycoprotein H | envelope glycoprotein H [Human herpesvirus 5] | AFR56062.1 |
| Glycoprotein H | envelope glycoprotein H [Human herpesvirus 5] | ACS92000.1 |
| Glycoprotein H | envelope glycoprotein H [Human herpesvirus 5] | AKI15316.1 |
| Glycoprotein H | envelope glycoprotein H [Human herpesvirus 5] | AFR54893.1 |
| Glycoprotein H | envelope glycoprotein H [Human herpesvirus 5] | AHJ86162.1 |
| Glycoprotein H | envelope glycoprotein H [Human herpesvirus 5] | ACS92165.1 |
| Glycoprotein H | envelope glycoprotein H [Human herpesvirus 5] | ACT81746.1 |
| Glycoprotein H | envelope glycoprotein H [Human herpesvirus 5] | AKI12305.1 |
| Glycoprotein H | envelope glycoprotein H [Human herpesvirus 5] | AKI09634.1 |
| Glycoprotein H | glycoprotein H [Human herpesvirus 5] | BAF44191.1 |
| Glycoprotein H | RecName: Full = Envelope glycoprotein H; | P17176.1 |
| | Short = gH; AltName: Full = Glycoprotein P86; | |
| | [Human herpesvirus 5 strain Towne] | |
| Glycoprotein H | envelope glycoprotein H [Human herpesvirus 5] | AKI13641.1 |
| Glycoprotein H | envelope glycoprotein H [Human herpesvirus 5] | AKI20832.1 |
| Glycoprotein H | envelope glycoprotein H [Human herpesvirus 5] | AKI09465.1 |
| Glycoprotein H | envelope glycoprotein H [Human herpesvirus 5] | ACS93407.1 |
| Glycoprotein H | envelope glycoprotein H [Human herpesvirus 5] | AKI07621.1 |
| Glycoprotein H | glycoprotein H [Human herpesvirus 5] | BAF44186.1 |
| Glycoprotein H | envelope glycoprotein H [Human herpesvirus 5] | AKI22834.1 |
| Glycoprotein H | envelope glycoprotein H [Human herpesvirus 5] | AKI14981.1 |
| Glycoprotein H | envelope glycoprotein H [Human herpesvirus 5] | AKI10139.1 |
| Glycoprotein H | envelope glycoprotein H [Human herpesvirus 5] | ACZ79822.1 |
| Glycoprotein H | envelope glycoprotein H [Human herpesvirus 5] | AAA45910.1 |
| Glycoprotein H | envelope glycoprotein H [Human herpesvirus 5] | AAA45913.1 |
| Glycoprotein H | glycoprotein H [Human herpesvirus 5] | BAF44188.1 |
| Glycoprotein H | envelope glycoprotein H [Human herpesvirus 5] | AKI18822.1 |
| Glycoprotein H | envelope glycoprotein H [Human herpesvirus 5] | AFR56229.1 |
| Glycoprotein H | envelope glycoprotein H [Human herpesvirus 5] | YP_081523.1 |
| Glycoprotein H | envelope glycoprotein H [Human herpesvirus 5] | AKI19826.1 |
| Glycoprotein H | envelope glycoprotein H [Human herpesvirus 5] | AAA45914.1 |
| Glycoprotein H | envelope glycoprotein H [Human herpesvirus 5] | AKI23334.1 |
| Glycoprotein H | envelope glycoprotein H [Human herpesvirus 5] | AKI14141.1 |
| Glycoprotein H | envelope glycoprotein H [Human herpesvirus 5] | AHB19545.1 |
| Glycoprotein H | envelope glycoprotein H [Human herpesvirus 5] | ACU83725.1 |
| Glycoprotein H | envelope glycoprotein H [Human herpesvirus 5] | AKI17318.1 |
| Glycoprotein H | envelope glycoprotein H [Human herpesvirus 5] | AKI13975.1 |
| Glycoprotein L | RecName: Full = Envelope glycoprotein L; | Q68672.1 |
| | Flags: Precursor [Human herpesvirus 5 (strain 5040)] | |
| Glycoprotein L | RecName: Full = Envelope glycoprotein L; | Q68669.1 |
| | Flags: Precursor [Human herpesvirus 5 (strain 2387)] | |
| Glycoprotein L | envelope glycoprotein L [Human herpesvirus 5] | AKI08825.1 |
| Glycoprotein L | envelope glycoprotein L [Human herpesvirus 5] | ACS92032.1 |
| Glycoprotein L | envelope glycoprotein L [Human herpesvirus 5] | AHJ86194.1 |
| Glycoprotein L | RecName: Full = Envelope glycoprotein L; Flags: Precursor | P16832.2 |
| Glycoprotein L | RecName: Full = Envelope glycoprotein L; | Q68671.1 |
| | Flags: Precursor [Human herpesvirus 5 (strain 5035)] | |
| Glycoprotein L | envelope glycoprotein L [Human herpesvirus 5] | AHB20074.1 |
| Glycoprotein L | envelope glycoprotein L [Human herpesvirus 5] | AKI12337.1 |
| Glycoprotein L | RecName: Full = Envelope glycoprotein L; | Q68668.1 |
| | Flags: Precursor [Human herpesvirus 5 (strain 1042)] | |
| Glycoprotein L | envelope glycoprotein L [Human herpesvirus 5] | AKI23365.1 |
| Glycoprotein L | envelope glycoprotein L [Human herpesvirus 5] | AKI21032.1 |
| Glycoprotein L | envelope glycoprotein L [Human herpesvirus 5] | YP_081555.1 |
| Glycoprotein L | RecName: Full = Envelope glycoprotein L; | Q68670.1 |
| | Flags: Precursor [Human herpesvirus 5 (strain 4654)] | |
| Glycoprotein L | envelope glycoprotein L [Human herpesvirus 5] | AKI17850.1 |
| Glycoprotein L | envelope glycoprotein L [Human herpesvirus 5] | ACZ80183.1 |
| Glycoprotein L | envelope glycoprotein L [Human herpesvirus 5] | AKI11508.1 |
| Glycoprotein L | envelope glycoprotein L [Human herpesvirus 5] | AKI10171.1 |
| Glycoprotein L | RecName: Full = Envelope glycoprotein L; | Q68673.1 |
| | Flags: Precursor [Human herpesvirus 5 (strain 5160)] | |
| Glycoprotein L | RecName: Full = Envelope glycoprotein L; | Q68666.1 |
| | Flags: Precursor [Human herpesvirus 5 strain PT] | |
| Glycoprotein L | envelope glycoprotein L [Human herpesvirus 5] | AKI18350.1 |
| Glycoprotein L | envelope glycoprotein L [Human herpesvirus 5] | AIC80693.1 |
| Glycoprotein L | envelope glycoprotein L [Human herpesvirus 5] | AKI12003.1 |
| Glycoprotein L | envelope glycoprotein L [Human herpesvirus 5] | AKI15849.1 |
| Glycoprotein L | envelope glycoprotein L [Human herpesvirus 5] | AKI13336.1 |
| Glycoprotein L | envelope glycoprotein L [Human herpesvirus 5] | AKI10840.1 |
| Glycoprotein L | RecName: Full = Envelope glycoprotein L; | Q68667.1 |
| | Flags: Precursor | |
| Glycoprotein L | envelope glycoprotein L [Human herpesvirus 5] | AHV84055.1 |
| Glycoprotein L | envelope glycoprotein L [Human herpesvirus 5] | AKI07653.1 |
| Glycoprotein L | envelope glycoprotein L [Human herpesvirus 5] | AFR55425.1 |
| Glycoprotein L | envelope glycoprotein L [Human herpesvirus 5] | AKI15013.1 |
| Glycoprotein L | envelope glycoprotein L [Human herpesvirus 5] | ACT81943.1 |
| Glycoprotein L | envelope glycoprotein L [Human herpesvirus 5] | AKI21367.1 |
| Glycoprotein L | envelope glycoprotein L [Human herpesvirus 5] | NP_612739.1 |
| pp150 | tegument protein pp150 [Human herpesvirus 5] | ACZ79954.1 |
| pp150 | tegument protein pp150 [Human herpesvirus 5] | AFR56030.1 |
| pp150 | tegument protein pp150 [Human herpesvirus 5] | ACU83693.1 |
| pp150 | tegument protein pp150 [Human herpesvirus 5] | AKI12106.1 |
| pp150 | tegument protein pp150 [Human herpesvirus 5] | AKI19625.1 |
| pp150 | tegument protein pp150 [Human herpesvirus 5] | AFR55362.1 |
| pp150 | tegument protein pp150 [Human herpesvirus 5] | AKI14613.1 |
| pp150 | tegument protein pp150 [Human herpesvirus 5] | AKI07924.1 |
| pp150 | tegument protein pp150 [Human herpesvirus 5] | AIC80127.1 |
| pp150 | tegument protein pp150 [Human herpesvirus 5] | AHB19512.1 |
| pp150 | tegument protein pp150 [Human herpesvirus 5] | YP_081491.1 |
| pp150 | tegument protein pp150 [Human herpesvirus 5] | AKI16116.1 |
| pp150 | extended tegument protein pp150 [Human herpesvirus 5] | AII79810.1 |
| pp150 | tegument protein pp150 [Human herpesvirus 5] | AIC80629.1 |
| pp150 | tegument protein pp150 [Human herpesvirus 5] | AFR55862.1 |
| pp150 | tegument protein pp150 [Human herpesvirus 5] | AKI10942.1 |
| pp150 | tegument protein pp150 [Human herpesvirus 5] | ACS91968.1 |
| pp150 | tegument protein pp150 [Human herpesvirus 5] | AKI15451.1 |
| pp150 | tegument protein pp150 [Human herpesvirus 5] | ACS92133.1 |
| pp150 | tegument protein pp150 [Human herpesvirus 5] | AFR56364.1 |
| pp150 | tegument protein pp150 [Human herpesvirus 5] | AFR54694.1 |
| pp150 | tegument protein pp150 [Human herpesvirus 5] | AKI23468.1 |
| pp150 | tegument protein pp150 [Human herpesvirus 5] | AKI17619.1 |
| pp150 | tegument protein pp150 [Human herpesvirus 5] | ABR55527.1 |
| pp150 | tegument protein pp150 [Human herpesvirus 5] | AFR55193.1 |
| pp150 | tegument protein pp150 [Human herpesvirus 5] | AFR54534.1 |
| pp150 | tegument protein pp150 [Human herpesvirus 5] | AKI18789.1 |
| pp150 | tegument protein pp150 [Human herpesvirus 5] | AKI07588.1 |
| pp150 | tegument protein pp150 [Human herpesvirus 5] | AKI22466.1 |
| pp150 | tegument protein pp150 [Human herpesvirus 5] | AKI20463.1 |
| pp150 | tegument protein pp150 [Human herpesvirus 5] | AKI14780.1 |
| pp150 | tegument protein pp150 [Human herpesvirus 5] | AKI15116.1 |
| pp150 | tegument protein pp150 [Human herpesvirus 5] | AKI14445.1 |
| pp150 | tegument protein pp150 [Human herpesvirus 5] | AKI22633.1 |
| pp150 | tegument protein pp150 [Human herpesvirus 5] | AKI09096.1 |
| pp150 | tegument protein pp150 [Human herpesvirus 5] | AKI13271.1 |
| pp150 | tegument protein pp150 [Human herpesvirus 5] | AKI08760.1 |
| pp150 | tegument protein pp150 [Human herpesvirus 5] | AFR56197.1 |
| pp150 | tegument protein pp150 [Human herpesvirus 5] | AFR54861.1 |
| pp150 | tegument protein pp150 [Human herpesvirus 5] | ACZ80119.1 |
| pp150 | tegument protein pp150 [Human herpesvirus 5] | AKI19960.1 |
| pp150 | tegument protein pp150 [Human herpesvirus 5] | AKI21134.1 |
| pp150 | tegument protein pp150 [Human herpesvirus 5] | AKI11938.1 |
| pp150 | tegument protein pp150 [Human herpesvirus 5] | AKI20128.1 |
| pp150 | tegument protein pp150 [Human herpesvirus 5] | AKI08928.1 |
| pp150 | tegument protein pp150 [Human herpesvirus 5] | ACZ80284.1 |
| pp150 | tegument protein pp150 [Human herpesvirus 5] | AKI21302.1 |
| pp150 | tegument protein pp150 [Human herpesvirus 5] | AKI12272.1 |
| pp150 | tegument protein pp150 [Human herpesvirus 5] | AKI20967.1 |
| pp150 | tegument protein pp150 [Human herpesvirus 5] | AKI19793.1 |
| pp150 | tegument protein pp150 [Human herpesvirus 5] | AKI23136.1 |
| pp150 | tegument protein pp150 [Human herpesvirus 5] | AKI10106.1 |
| pp150 | tegument protein pp150 [Human herpesvirus 5] | AKI11772.1 |
| pp150 | tegument protein pp150 [Human herpesvirus 5] | AKI08591.1 |
| pp150 | tegument protein pp150 [Human herpesvirus 5] | AKI11443.1 |
| pp150 | tegument protein pp150 [Human herpesvirus 5] | AKI14948.1 |
| pp150 | tegument protein pp150 [Human herpesvirus 5] | ADE88040.1 |
| pp150 | tegument protein pp150 [Human herpesvirus 5] | AKI22969.1 |
| pp150 | tegument protein pp150 [Human herpesvirus 5] | AHJ86130.1 |
| pp150 | tegument protein pp150 [Human herpesvirus 5] | ACT81879.1 |
| pp150 | tegument protein pp150 [Human herpesvirus 5] | AKI15950.1 |
| pp150 | tegument protein pp150 [Human herpesvirus 5] | AKI15617.1 |
| pp150 | tegument protein pp150 [Human herpesvirus 5] | AHB19679.1 |
| pp150 | tegument protein pp150 [Human herpesvirus 5] | AHB19344.1 |
| pp150 | tegument protein pp150 [Human herpesvirus 5] | ACT81714.1 |
| pp150 | tegument protein pp150 [Human herpesvirus 5] | AKI07756.1 |
| pp150 | tegument protein pp150 [Human herpesvirus 5] | AKI11607.1 |
| pp150 | tegument protein pp150 [Human herpesvirus 5] | AIC80295.1 |
| pp150 | tegument protein pp150 [Human herpesvirus 5] | AKI11275.1 |
| pp150 | tegument protein pp150 [Human herpesvirus 5] | AHB20010.1 |
| pp150 | tegument protein pp150 [Human herpesvirus 5] | AIC80463.1 |
| pp150 | tegument protein pp150 [Human herpesvirus 5] | AKI17952.1 |
| pp150 | tegument protein pp150 [Human herpesvirus 5] | ACM48022.1 |
| pp150 | tegument protein pp150 [Human herpesvirus 5] | AKI16285.1 |
| pp150 | tegument protein pp150 [Human herpesvirus 5] | AKI09601.1 |
| pp150 | tegument protein pp150 [Human herpesvirus 5] | AKI22299.1 |
| pp150 | tegument protein pp150 [Human herpesvirus 5] | AHV83990.1 |
| pp150 | tegument protein pp150 [Human herpesvirus 5] | AKI23301.1 |
| pp150 | tegument protein pp150 [Human herpesvirus 5] | AGL96632.1 |
| pp150 | tegument protein pp150 [Human herpesvirus 5] | AKI18285.1 |
| pp150 | tegument protein pp150 [Human herpesvirus 5] | ADV04360.1 |
| pp65 | tegument protein pp65 [Human herpesvirus 5] | ACZ79994.1 |
| pp65 | tegument protein pp65 [Human herpesvirus 5] | ACS92173.1 |
| pp65 | tegument protein pp65 [Human herpesvirus 5] | AKI09642.1 |
| pp65 | tegument protein pp65 [Human herpesvirus 5] | AKI16326.1 |
| pp65 | tegument protein pp65 [Human herpesvirus 5] | ADD39129.1 |
| pp65 | tegument protein pp65 [Human herpesvirus 5] | ACM48061.1 |
| pp65 | tegument protein pp65 [Human herpesvirus 5] | AKI20001.1 |
| pp65 | tegument protein pp65 [Human herpesvirus 5] | AKI14149.1 |
| pp65 | tegument protein pp65 [Human herpesvirus 5] | AHB19720.1 |
| pp65 | tegument protein pp65 [Human herpesvirus 5] | ADV04400.1 |
| pp65 | tegument protein pp65 [Human herpesvirus 5] | AKI21507.1 |
| pp65 | tegument protein pp65 [Human herpesvirus 5] | AKI15825.1 |
| pp65 | tegument protein pp65 [Human herpesvirus 5] | AKI08299.1 |
| pp65 | tegument protein pp65 [Human herpesvirus 5] | AKI07965.1 |
| pp65 | tegument protein pp65 [Human herpesvirus 5] | AKI22339.1 |
| pp65 | tegument protein pp65 [Human herpesvirus 5] | AKI12978.1 |
| pp65 | tegument protein pp65 [Human herpesvirus 5] | AKI11979.1 |
| pp65 | tegument protein pp65 [Human herpesvirus 5] | AHB19886.1 |
| pp65 | tegument protein pp65 [Human herpesvirus 5] | YP_081531.1 |
| pp65 | tegument protein pp65 [Human herpesvirus 5] | AKI23010.1 |
| pp65 | tegument protein pp65 [Human herpesvirus 5] | AKI10983.1 |
| pp65 | tegument protein pp65 [Human herpesvirus 5] | AKT10314.1 |
| pp65 | tegument protein pp65 [Human herpesvirus 5] | AFR56070.1 |
| pp65 | 65K lower matrix phosphoprotein-human | WMBETW |
| | cytomegalovirus (strain Towne) | |
| pp65 | mutant UL83 [Human herpesvirus 5] | AAP59842.1 |
| pp65 | tegument protein pp65 [Human herpesvirus 5] | AKI14317.1 |
| pp65 | tegument protein pp65 [Human herpesvirus 5] | AFR54574.1 |
| pp65 | tegument protein pp65 [Human herpesvirus 5] | AFR56237.1 |
| pp65 | tegument protein pp65 [Human herpesvirus 5] | AKI18326.1 |
| pp65 | tegument protein pp65 [Human herpesvirus 5] | AKI22842.1 |
| pp65 | tegument protein PP65 [Human herpesvirus 5] | AHV84031.1 |
| pp65 | tegument protein [synthetic construct] | AAT68258.1 |
| pp65 | phosphorylated matrix protein (pp65) [Human herpesvirus 5] | AAA45996.1 |
| pp65 | tegument protein pp65 [Panine herpesvirus 2] | NP_612716.1 |
| pp65 | tegument protein pp65 [Human herpesvirus 5] | ADJ68256.1 |
| pp65 | tegument protein pp65 [Human herpesvirus 5] | ADJ68266.1 |
| UL100 (gM) | envelope glycoprotein M [Human herpesvirus 5] | ACT81935.1 |
| UL100 (gM) | envelope glycoprotein M [Human herpesvirus 5] | YP_081547.1 |
| UL100 (gM) | envelope glycoprotein M [Human herpesvirus 5] | ACM48077.1 |
| UL100 (gM) | RecName: Full = Envelope glycoprotein M; Short = gM | P16733.1 |
| UL100 (gM) | envelope glycoprotein M [Human herpesvirus 5] | AKI18175.1 |
| UL100 (gM) | envelope glycoprotein M [Human herpesvirus 5] | AFR54590.1 |
| UL100 (gM) | envelope glycoprotein M [Human herpesvirus 5] | AKI20017.1 |
| UL100 (gM) | envelope glycoprotein M [Human herpesvirus 5] | AKI09994.1 |
| UL100 (gM) | UL100 [Human herpesvirus 5] | AAS48986.1 |
| UL100 (gM) | envelope glycoprotein M [Human herpesvirus 5] | AKI20856.1 |
| UL100 (gM) | envelope glycoprotein M [Human herpesvirus 5] | AKI14333.1 |
| UL100 (gM) | envelope glycoprotein M [Human herpesvirus 5] | AHB19736.1 |
| UL100 (gM) | envelope glycoprotein M [Human herpesvirus 5] | AGT36389.1 |
| UL100 (gM) | envelope glycoprotein M [Human herpesvirus 5] | ACZ80175.1 |
| UL100 (gM) | envelope glycoprotein M [Human herpesvirus 5] | AKI18009.1 |
| UL100 (gM) | envelope glycoprotein M [Human herpesvirus 5] | AKI23358.1 |
| UL100 (gM) | envelope glycoprotein M [Human herpesvirus 5] | AHV84047.1 |
| UL100 (gM) | envelope glycoprotein M [Human herpesvirus 5] | ACS92024.1 |
| UL100 (gM) | envelope glycoprotein M [Human herpesvirus 5] | AKI10999.1 |
| UL100 (gM) | envelope glycoprotein M [Human herpesvirus 5] | AKI16173.1 |
| UL100 (gM) | envelope glycoprotein M [Human herpesvirus 5] | AFR54917.1 |
| UL100 (gM) | UL100 [Human herpesvirus 5] | ABV71622.1 |
| UL100 (gM) | envelope glycoprotein M [Human herpesvirus 5] | AKI13999.1 |
| UL100 (gM) | envelope glycoprotein M [Human herpesvirus 5] | AKI12329.1 |
| UL100 (gM) | envelope glycoprotein M [Human herpesvirus 5] | AKI21523.1 |
| UL100 (gM) | envelope glycoprotein M [Human herpesvirus 5] | AKI18342.1 |
| UL100 (gM) | envelope glycoprotein M [Human herpesvirus 5] | AKI09658.1 |
| UL100 (gM) | envelope glycoprotein M [Human herpesvirus 5] | AKI07813.1 |
| UL100 (gM) | envelope glycoprotein M [Human herpesvirus 5] | AKI18846.1 |
| UL100 (gM) | envelope glycoprotein M [Human herpesvirus 5] | AFR55081.1 |
| UL100 (gM) | envelope glycoprotein M [Human herpesvirus 5] | AKI17342.1 |
| UL123 | regulatory protein IE1 [Human herpesvirus 5] | ACT81950.1 |
| UL123 | regulatory protein IE1 [Human herpesvirus 5] | AKI07996.1 |
| UL123 | regulatory protein IE1 [Human herpesvirus 5] | AAR31361.1 |
| UL123 | regulatory protein IE1 [Human herpesvirus 5] | AKI09840.1 |
| UL123 | regulatory protein IE1 [Human herpesvirus 5] | AKI12010.1 |
| UL123 | regulatory protein IE1 [Human herpesvirus 5] | AAR31390.1 |
| UL123 | regulatory protein IE1 [Human herpesvirus 5] | AFR55598.1 |
| UL123 | regulatory protein IE1 [Human herpesvirus 5] | AKI08832.1 |
| UL123 | immediate early transcriptional regulator [Human herpesvirus 5] | ACL27071.1 |
| UL123 | regulatory protein IE1 [Human herpesvirus 5] | AHB19584.1 |
| UL123 | regulatory protein IE1 [Human herpesvirus 5] | AKI18861.1 |
| UL123 | regulatory protein IE1 [Human herpesvirus 5] | AAR31419.1 |
| UL123 | regulatory protein IE1 [Human herpesvirus 5] | AKI23372.1 |
| UL123 | regulatory protein IE1 [Human herpesvirus 5] | AKI16357.1 |
| UL123 | regulatory protein IE1 [Human herpesvirus 5] | AKI10512.1 |
| UL123 | regulatory protein IE1 [Human herpesvirus 5] | AKI19028.1 |
| UL123 | regulatory protein IE1 [Human herpesvirus 5] | AKI11347.1 |
| UL123 | 72 kDa immediate-early 1 protein [Human herpesvirus 5] | ACT34667.1 |
| UL123 | regulatory protein IE1 [Human herpesvirus 5] | ADB84698.1 |
| UL123 | immediate early transcriptional regulator [Human herpesvirus 5] | ACL27072.1 |
| UL123 | regulatory protein IE1 [Human herpesvirus 5] | AKI22873.1 |
| UL123 | regulatory protein IE1 [Human herpesvirus 5] | AKI20200.1 |
| UL123 | regulatory protein IE1 [Human herpesvirus 5] | AKI12677.1 |
| UL123 | regulatory protein IE1 [Human herpesvirus 5] | AHV84062.1 |
| UL123 | regulatory protein IE1 [Human herpesvirus 5] | AFR55096.1 |
| UL123 | regulatory protein IE1 [Human herpesvirus 5] | AFR54932.1 |
| UL123 | regulatory protein IE1 [Human herpesvirus 5] | AKI22205.1 |
| UL123 | regulatory protein IE1 [Human herpesvirus 5] | AFR55264.1 |
| UL123 | regulatory protein IE1 [Human herpesvirus 5] | AKI18357.1 |
| UL123 | regulatory protein IE1 [Human herpesvirus 5] | AKI17188.1 |
| UL123 | regulatory protein IE1 [Human herpesvirus 5] | AKI12841.1 |
| UL123 | regulatory protein IE1 [Human herpesvirus 5] | AKI09673.1 |
| UL123 | regulatory protein IE1 [Human herpesvirus 5] | AKI21537.1 |
| UL123 | regulatory protein IE1 [Human herpesvirus 5] | AKI20871.1 |
| UL123 | regulatory protein IE1 [Human herpesvirus 5] | AGL96703.1 |
| UL123 | regulatory protein IE1 [Human herpesvirus 5] | AAR31477.1 |
| UL123 | regulatory protein IE1 [Human herpesvirus 5] | AKI21374.1 |
| UL123 | regulatory protein IE1 [Human herpesvirus 5] | ACZ80025.1 |
| UL123 | 72 kDa immediate-early 1 protein [Human herpesvirus 5] | ACT34666.1 |
| UL123 | regulatory protein IE1 [Human herpesvirus 5] | AKI20032.1 |
| UL123 | regulatory protein IE1 [Human herpesvirus 5] | AAR31303.1 |
| UL123 | regulatory protein IE1 [Human herpesvirus 5] | AIC80700.1 |
| UL123 | regulatory protein IE1 [Human herpesvirus 5] | ADB84746.1 |
| UL123 | immediate early transcriptional regulator [Human herpesvirus 5] | ACL27084.1 |
| UL123 | regulatory protein IE1 [Human herpesvirus 5] | ADV04431.1 |
| UL123 | regulatory protein IE1 [Human herpesvirus 5] | AKI11515.1 |
| UL123 | regulatory protein IE1 [Human herpesvirus 5] | AFR56435.1 |
| UL123 | immediate early transcriptional regulator [Human herpesvirus 5] | ACL27074.1 |
| UL123 | regulatory protein IE1 [Human herpesvirus 5] | AKI14180.1 |
| UL123 | regulatory protein IE1 [Human herpesvirus 5] | AKI07828.1 |
| UL123 | regulatory protein IE1 [Human herpesvirus 5] | AHB19751.1 |
| UL123 | regulatory protein IE1 [Human herpesvirus 5] | ADB84818.1 |
| UL123 | regulatory protein IE1 [Human herpesvirus 5] | AKI18526.1 |
| UL123 | regulatory protein IE1 [Human herpesvirus 5] | AKI14014.1 |
| UL123 | regulatory protein IE1 [Human herpesvirus 5] | ACT81785.1 |
| UL123 | regulatory protein IE1 [Human herpesvirus 5] | ADB44102.1 |
| UL123 | regulatory protein IE1 [Human herpesvirus 5] | AKI15187.1 |
| UL123 | immediate early transcriptional regulator [Human herpesvirus 5] | ACL27092.1 |
| UL123 | immediate early transcriptional regulator [Human herpesvirus 5] | ACL27056.1 |
| UL123 | regulatory protein IE1 [Human herpesvirus 5] | AKI18024.1 |
| UL123 | regulatory protein IE1 [Human herpesvirus 5] | AKI14517.1 |
| UL123 | regulatory protein IE1 [Human herpesvirus 5] | ADB88106.1 |
| UL123 | regulatory protein IE1 [Human herpesvirus 5] | AKH5355.1 |
| UL123 | regulatory protein IE1 [Human herpesvirus 5] | AKI10178.1 |
| UL123 | regulatory protein IE1 [Human herpesvirus 5] | ADB84722.1 |
| UL123 | regulatory protein IE1 [Human herpesvirus 5] | ADB84650.1 |
| UL123 | regulatory protein IE1 [Human herpesvirus 5] | AFR56268.1 |
| UL123 | regulatory protein IE1 [Human herpesvirus 5] | YP_081562.1 |
| UL123 | regulatory protein IE1 [Human herpesvirus 5] | AKI22538.1 |
| UL123 | regulatory protein IE1 [Human herpesvirus 5] | AHB19917.1 |
| UL123 | regulatory protein IE1 [Human herpesvirus 5] | AAR31332.1 |
| UL123 | RecName: Full = 55 kDa immediate-early protein 1; Short = IE1 | P13202.1 |
| UL123 | regulatory protein IE1 [Human herpesvirus 5] | AAR31448.1 |
| UL123 | regulatory protein IE1 [Human herpesvirus 5] | AKI14852.1 |
| UL123 | regulatory protein IE1 [Human herpesvirus 5] | AKI14348.1 |
| UL123 | immediate early transcriptional regulator [Human herpesvirus 5] | ACL27066.1 |
| UL123 | immediate early transcriptional regulator [Human herpesvirus 5] | ACL27058.1 |
| UL123 | immediate early transcriptional regulator [Human herpesvirus 5] | ACL27086.1 |
| UL123 | regulatory protein IE1 [Human herpesvirus 5] | AKI16022.1 |
| UL123 | regulatory protein IE1 [Human herpesvirus 5] | AIC80534.1 |
| UL123 | immediate early transcriptional regulator [Human herpesvirus 5] | ACL27094.1 |
| UL123 | immediate early transcriptional regulator [Human herpesvirus 5] | ACL27093.1 |
| UL123 | immediate early transcriptional regulator [Human herpesvirus 5] | ACL27073.1 |
| UL123 | pp65/IE1 fusion protein [synthetic construct] | ABQ23593.1 |
| UL123 | regulatory protein IE1 [Human herpesvirus 5] | ACS92204.1 |
| UL123 | RecName: Full = 55 kDa immediate-early protein 1; | P03169.1 |
| | Short-IE1 [Human herpesvirus 5 strain Towne] | |
| UL123 | regulatory protein IE1 [Human herpesvirus 5] | ADB84794.1 |
| UL123 | major immediate-early protein [Human herpesvirus 5] | AAA45979.1 |
| UL123 | immediate early transcriptional regulator [Human herpesvirus 5] | ACL27059.1 |
| UL123 | regulatory protein IE1 [Human herpesvirus 5] | AKI21039.1 |
| UL123 | immediate early transcriptional regulator [Human herpesvirus 5] | ACL27065.1 |
| UL123 | immediate early transcriptional regulator [Human herpesvirus 5] | ACL27087.1 |
| UL123 | regulatory protein IE1 [Human herpesvirus 5] | AAR31504.1 |
| UL123 | immediate early transcriptional regulator [Human herpesvirus 5] | ACL27082.1 |
| UL123 | immediate early transcriptional regulator [Human herpesvirus 5] | ACL27055.1 |
| UL123 | immediate early transcriptional regulator [Human herpesvirus 5] | ACL27081.1 |
| UL123 | regulatory protein IE1 [Human herpesvirus 5] | AKI12344.1 |
| UL123 | immediate early transcriptional regulator [Human herpesvirus 5] | ACL27089.1 |
| UL123 | immediate early transcriptional regulator [Human herpesvirus 5] | ACL27062.1 |
| UL123 | immediate early transcriptional regulator [Human herpesvirus 5] | ACL27057.1 |
| UL128 | envelope protein UL128 [Human herpesvirus 5] | AAR31451.1 |
| UL128 | UL128 [Human herpesvirus 5] | ABA86617.1 |
| UL128 | envelope protein UL128 [Human herpesvirus 5] | AAR31335.1 |
| UL128 | envelope protein UL128 [Human herpesvirus 5] | ADV04433.1 |
| UL128 | UL128 [Human herpesvirus 5] | ADF30829.1 |
| UL128 | envelope protein UL128 [Human herpesvirus 5] | ACS92206.1 |
| UL128 | envelope protein UL128 [Human herpesvirus 5] | ADB84652.1 |
| UL128 | envelope protein UL128 [Human herpesvirus 5] | AHJ86203.1 |
| UL128 | envelope protein UL128 [Human herpesvirus 5] | AAO11759.2 |
| UL128 | envelope protein UL128 [Human herpesvirus 5] | AKI07662.1 |
| UL128 | UL128 [Human herpesvirus 5] | ABA86608.1 |
| UL128 | envelope protein UL128 [Human herpesvirus 5] | AKI12512.1 |
| UL128 | envelope protein UL128 [Human herpesvirus 5] | AKI21705.1 |
| UL128 | envelope protein UL128 [Human herpesvirus 5] | AKI20034.1 |
| UL128 | RecName: Full = Uncharacterized protein UL128 | P16837.2 |
| UL128 | UL128 [Human herpesvirus 5] | ABA86623.1 |
| UL128 | envelope protein UL128 [Human herpesvirus 5] | AKI16857.1 |
| UL128 | envelope protein UL128 [Human herpesvirus 5] | AKI18528.1 |
| UL128 | envelope protein UL128 [Human herpesvirus 5] | ADB84820.1 |
| UL128 | envelope protein UL128 [Human herpesvirus 5] | AAR31422.1 |
| UL128 | envelope protein UL128 [Human herpesvirus 5] | AGL96705.1 |
| UL128 | envelope protein UL128 [Human herpesvirus 5] | ACT81952.1 |
| UL128 | envelope protein UL128 [Human herpesvirus 5] | AKI18359.1 |
| UL128 | UL128 [Human herpesvirus 5] | AAO11775.2 |
| UL128 | UL128 [Human herpesvirus 5] | ABA86605.1 |
| UL128 | UL128 [Human herpesvirus 5] | ADF30833.1 |
| UL128 | envelope protein UL128 [Human herpesvirus 5] | AKI11182.1 |
| UL128 | envelope protein UL128 [Human herpesvirus 5] | AKI15691.1 |
| UL128 | UL128 [Human herpesvirus 5] | ABA86622.1 |
| UL128 | UL128 [Human herpesvirus 5] | ADF30832.1 |
| UL128 | UL128 [Human herpesvirus 5] | ABA86616.1 |
| UL128 | envelope protein UL128 [Human herpesvirus 5] | AAO11755.2 |
| UL128 | envelope protein UL128 [Human herpesvirus 5] | AFR55266.1 |
| UL128 | UL128 [Human herpesvirus 5] | ABA86618.1 |
| UL128 | envelope protein UL128 [Human herpesvirus 5] | ADB84700.1 |
| UL128 | UL128 [Human herpesvirus 5] | ADE62337.1 |
| UL128 | UL128 [Human herpesvirus 5] | ADF30837.1 |
| UL128 | UL128 [Human herpesvirus 5] | ABA86604.1 |
| UL128 | envelope protein UL128 [Human herpesvirus 5] | AKI21208.1 |
| UL128 | UL128 [Human herpesvirus 5] | ABA86609.1 |
| UL128 | envelope protein UL128 [Human herpesvirus 5] | AKI10514.1 |
| UL128 | truncated UL128 protein [Human herpesvirus 5] | ADG36331.1 |
| UL128 | HCMVUL128 [Human herpesvirus 5] | CAA35330.1 |
| UL130 | UL130 [Human herpesvirus 5] | ABA86653.1 |
| UL130 | UL130 [Human herpesvirus 5] | ABA86666.1 |
| UL130 | UL130 [Human herpesvirus 5] | ABA86652.1 |
| UL130 | envelope glycoprotein UL130 [Human herpesvirus 5] | YP_081565.1 |
| UL130 | envelope glycoprotein UL130 [Human herpesvirus 5] | AKI08835.1 |
| UL130 | UL130 [Human herpesvirus 5] | AAY33781.1 |
| UL130 | envelope glycoprotein UL130 [Human herpesvirus 5] | ACS92042.1 |
| UL130 | envelope glycoprotein UL130 [Human herpesvirus 5] | AKI10515.1 |
| UL130 | envelope glycoprotein UL130 [Human herpesvirus 5] | AKI07663.1 |
| UL130 | envelope glycoprotein UL130 [Human herpesvirus 5] | AHB19754.1 |
| UL130 | envelope glycoprotein UL130 [Human herpesvirus 5] | AFR55435.1 |
| UL130 | UL130 [Human herpesvirus 5] | AAY33778.1 |
| UL130 | envelope glycoprotein UL130 [Human herpesvirus 5] | AKI18864.1 |
| UL130 | envelope glycoprotein UL130 [Human herpesvirus 5] | AIC80537.1 |
| UL130 | envelope glycoprotein UL130 [Human herpesvirus 5] | ADB44105.1 |
| UL130 | envelope glycoprotein UL130 [Human herpesvirus 5] | ADB84797.1 |
| UL130 | envelope glycoprotein UL130 [Human herpesvirus 5] | AKI22373.1 |
| UL130 | envelope glycoprotein UL130 [Human herpesvirus 5] | AGL96706.1 |
| UL130 | envelope glycoprotein UL130 [Human herpesvirus 5] | AKI22042.1 |
| UL130 | envelope glycoprotein UL130 [Human herpesvirus 5] | AHJ86204.1 |
| UL130 | RecName: Full = Uncharacterized protein UL130 | P16772.1 |
| UL130 | envelope glycoprotein UL130 [Human herpesvirus 5] | AKI20706.1 |
| UL130 | UL130 [Human herpesvirus 5] | ABA86662.1 |
| UL130 | UL130 [Human herpesvirus 5] | ABA86665.1 |
| UL130 | UL130 [Human herpesvirus 5] | ABA86659.1 |
| UL130 | UL130 [Human herpesvirus 5] | ADF30831.1 |
| UL130 | orf UL130 [Human herpesvirus 5] | AAA85889.1 |
| UL130 | envelope glycoprotein UL130 [Human herpesvirus 5] | AKI11183.1 |
| UL130 | envelope glycoprotein UL130 [Human herpesvirus 5] | AKI19031.1 |
| UL130 | UL130 [Human herpesvirus 5] | ADE62342.1 |
| UL130 | envelope glycoprotein UL130 [Human herpesvirus 5] | AFR55099.1 |
| UL130 | envelope glycoprotein UL130 [Human herpesvirus 5] | AAR31336.1 |
| UL130 | UL130 [Human herpesvirus 5] | ABA86654.1 |
| UL130 | envelope glycoprotein UL130 [Human herpesvirus 5] | AKI17191.1 |
| UL130 | envelope glycoprotein UL130 [Human herpesvirus 5] | ADB84701.1 |
| UL130 | UL130 [Human herpesvirus 5] | ADF30838.1 |
| UL130 | envelope glycoprotein UL130 [Human herpesvirus 5] | AKI15859.1 |
| UL130 | UL130 [Human herpesvirus 5] | ABA86605.1 |
| UL130 | envelope glycoprotein UL130 [Human herpesvirus 5] | AFR55267.1 |
| UL130 | envelope glycoprotein UL130 [Human herpesvirus 5] | ACS92207.1 |
| UL130 | envelope glycoprotein UL130 [Human herpesvirus 5] | AAR31307.1 |
| UL130 | envelope glycoprotein UL130 [Human herpesvirus 5] | AKI15358.1 |
| UL130 | envelope glycoprotein UL130 [Human herpesvirus 5] | AKI21042.1 |
| UL130 | envelope glycoprotein UL130 [Human herpesvirus 5] | AKI16360.1 |
| UL130 | UL130 [Human herpesvirus 5] | ABA86661.1 |
| UL130 | envelope glycoprotein UL130 [Human herpesvirus 5] | AHB19920.1 |
| UL130 | UL130 [Human herpesvirus 5] | ABV71640.1 |
| UL130 | envelope glycoprotein UL130 [Human herpesvirus 5] | AKI23375.1 |
| UL130 | envelope glycoprotein UL130 [Human herpesvirus 5] | AKI08333.1 |
| UL130 | envelope glycoprotein UL130 [Human herpesvirus 5] | AKI11377.1 |
| UL130 | envelope glycoprotein UL130 [Human herpesvirus 5] | AKI16526.1 |
| UL130 | UL130 [Human herpesvirus 5] | ADE62336.1 |
| UL130 | envelope glycoprotein UL130 [Human herpesvirus 5] | AKI14017.1 |
| UL130 | envelope glycoprotein UL130 [Human herpesvirus 5] | AKI09843.1 |
| UL130 | mutant fusion protein [Human herpesvirus 5] | ADE62322.1 |
| UL130 | envelope glycoprotein UL130 [Human herpesvirus 5] | AKI12013.1 |
| UL130 | envelope glycoprotein UL130 [Human herpesvirus 5] | AKI20371.1 |
| UL130 | envelope glycoprotein UL130 [Human herpesvirus 5] | ACZ81666.1 |
| UL130 | envelope glycoprotein UL130 [Human herpesvirus 5] | AAR31365.1 |
| UL130 | envelope glycoprotein UL130 [Human herpesvirus 5] | AAO11754.1 |
| UL131A | envelope protein UL131A [Human herpesvirus 5] | YP_081566.1 |
| UL131A | envelope protein UL131A [Human herpesvirus 5] | AKI12514.1 |
| UL131A | envelope protein UL131A [Human herpesvirus 5] | AKI11683.1 |
| UL131A | envelope protein UL131A [Human herpesvirus 5] | AAO11766.1 |
| UL131A | UL131A [Human herpesvirus 5] | ABA86643.1 |
| U1131A | UL131A [Human herpesvirus 5] | ADE62341.1 |
| UL131A | truncated envelope protein UL131A [Human herpesvirus 5] | ADV04435.1 |
| UL131A | envelope protein UL131A [Human herpesvirus 5] | AFR56272.1 |
| UL131A | envelope protein UL131A [Human herpesvirus 5] | AKI11018.1 |
| UL131A | envelope protein UL131A [Human herpesvirus 5] | AHB19755.1 |
| UL131A | envelope protein UL131A [Human herpesvirus 5] | AKT12348.1 |
| UL131A | UL131a protein [Human herpesvirus 5] | ADG36333.1 |
| UL131A | UL131A [Human herpesvirus 5] | ABA86640.1 |
| U1131A | envelope protein UL131A [Human herpesvirus 5] | AKI08836 1 |
| UL131A | UL131A [Human herpesvirus 5] | ABA86639.1 |
| UL131A | envelope protein UL131A [Human herpesvirus 5] | AFR55436.1 |
| UL131A | envelope protein UL131A [Human herpesvirus 5] | ADB84774.1 |
| UL131A | envelope protein UL131A [Human herpesvirus 5] | ADB84822.1 |
| UL131A | UL131A [Human herpesvirus 5] | ADF30839.1 |
| UL131A | UL131A [Human herpesvirus 5] | ABA86648.1 |
| UL131A | UL131A [Human herpesvirus 5] | ABA86635.1 |
| UL131A | envelope protein UL131A [Human herpesvirus 5] | AFR55436.1 |
| U1131A | UL131A [Human herpesvirus 5] | ABA86637.1 |
| UL131A | UL131A [Human herpesvirus 5] | ABA86644.1 |
| UL131A | UL131A [Human herpesvirus 5] | ABA86647.1 |
| UL131A | UL131A [Human herpesvirus 5] | ABA86629.1 |
| UL131A | UL131A [Human herpesvirus 5] | ABA86630.1 |
| UL131A | UL131A [Human herpesvirus 5] | ABA86646.1 |
| UL55 (gB) | envelope glycoprotein B [Human herpesvirus 5] | ACS91991.1 |
| UL55 (gB) | envelope glycoprotein B [Human herpesvirus 5] | AKI12129.1 |
| UL55 (gB) | envelope glycoprotein B [Human herpesvirus 5] | ACZ79977.1 |
| UL55 (gB) | envelope glycoprotein B [Human herpesvirus 5] | AFR55216.1 |
| UL55 (gB) | envelope glycoprotein B [Human herpesvirus 5] | AKI22656.1 |
| UL55 (gB) | glycoprotein B [Human herpesvirus 5] | AAA45934.1 |
| UL55 (gB) | envelope glycoprotein B [Human herpesvirus 5] | AFR54884.1 |
| UL55 (gB) | envelope glycoprotein B [Human herpesvirus 5] | AKI22156.1 |
| UL55 (gB) | envelope glycoprotein B [Human herpesvirus 5] | AKI14299.1 |
| UL55 (gB) | envelope glycoprotein B [Human herpesvirus 5] | ADV04383.1 |
| UL55 (gB) | envelope glycoprotein B [Human herpesvirus 5] | AKI20990.1 |
| UL55 (gB) | envelope glycoprotein B [Human herpesvirus 5] | AKI09624.1 |
| UL55 (gB) | envelope glycoprotein B [Human herpesvirus 5] | ADD39116.1 |
| UL55 (gB) | envelope glycoprotein B [Human herpesvirus 5] | ACT81737.1 |
| UL55 (gB) | envelope glycoprotein B [Human herpesvirus 5] | AKI11131.1 |
| UL55 (gB) | envelope glycoprotein B [Human herpesvirus 5] | AKI17642.1 |
| UL55 (gB) | envelope glycoprotein B [Human herpesvirus 5] | AIC80652.1 |
| UL55 (gB) | envelope glycoprotein B [Human herpesvirus 5] | AFR55719.1 |
| UL55 (gB) | envelope glycoprotein B [Human herpesvirus 5] | AKI09288.1 |
| UL55 (gB) | glycoprotein B [Human herpesvirus 5] | AAA45930.1 |
| UL55 (gB) | envelope glycoprotein B [Human herpesvirus 5] | AKI12960.1 |
| UL55 (gB) | glycoprotein B [Human herpesvirus 5] | AAA45926.1 |
| UL55 (gB) | glycoprotein B [Human herpesvirus 5] | AAA45925.1 |
| UL55 (gB) | envelope glycoprotein B [Human herpesvirus 5] | AKI80437.1 |
| UL55 (gB) | envelope glycoprotein B [Human herpesvirus 5] | AKI22824.1 |
| UL55 (gB) | envelope glycoprotein B [Human herpesvirus 5] | AHV84013.1 |
| UL55 (gB) | envelope glycoprotein B [Human herpesvirus 5] | AKI07947.1 |
| UL55 (gB) | envelope glycoprotein B [Human herpesvirus 5] | AFR54557.1 |
| UL55 (gB) | envelope glycoprotein B [Human herpesvirus 5] | AHB19702.1 |
| UL55 (gB) | RecName: Full = Envelope glycoprotein B; Short = gB; Flags: Precursor | P06473.1 |
| UL55 (gB) | glycoprotein B [Human herpesvirus 5] | ADB92600.1 |
| UL55 (gB) | envelope glycoprotein B [Human herpesvirus 5] | ADE88063.1 |
| UL55 (gB) | envelope glycoprotein B [Human herpesvirus 5] | AHJ86153.1 |
| UL55 (gB) | envelope glycoprotein B [Human herpesvirus 5] | AFR55885.1 |
| UL55 (gB) | UL55 [Human herpesvirus 5] | ABV71586.1 |
| UL55 (gB) | envelope glycoprotein B [Human herpesvirus 5] | ACS92156.1 |
| UL55 (gB) | envelope glycoprotein B [Human herpesvirus 5] | AKI23491.1 |
| UL55 (gB) | RecName: Full = Envelope glycoprotein B; Short = gB; | P13201.1 |
| | Contains: RecName: Full = Glycoprotein | |
| | GP55; Flags: Precursor | |
| UL55 (gB) | glycoprotein B [Human herpesvirus 5] | ABQ23592.1 |
| UL55 (gB) | glycoprotein B [Human herpesvirus 5] | AAB07485.1 |
| UL55 (gB) | envelope glycoprotein B [Human herpesvirus 5] | ACM48044.1 |
| UL55 (gB) | glycoprotein B [Human herpesvirus 5] | AAA45928.1 |
| UL55 (gB) | envelope glycoprotein B [Human herpesvirus 5] | ACS32370.1 |
| UL55 (gB) | envelope glycoprotein B [Human herpesvirus 5] | AKI19983.1 |
| UL55 (gB) | envelope glycoprotein B [Human herpesvirus 5] | AKI13294.1 |
| UL55 (gB) | envelope glycoprotein B [Human herpesvirus 5] | AFR55048.1 |
| UL55 (gB) | envelope glycoprotein B [Human herpesvirus 5] | AKI19483.1 |
| UL55 (gB) | envelope glycoprotein B [Human herpesvirus 5] | YP_081514.1 |
| UL55 (gB) | envelope glycoprotein B [Human herpesvirus 5] | AKI20319.1 |
| UL55 (gB) | envelope glycoprotein B [Human herpesvirus 5] | AHB20033.1 |
| UL55 (gB) | envelope glycoprotein B [Human herpesvirus 5] | AKI23324.1 |
| UL55 (gB) | envelope glycoprotein B [Human herpesvirus 5] | AKI13965.1 |
| UL55 (gB) | envelope glycoprotein B [Human herpesvirus 5] | ACS93398.1 |
| UL55 (gB) | envelope glycoprotein B [Human herpesvirus 5] | AKI08783.1 |
| UL55 (gB) | envelope glycoprotein B [Human herpesvirus 5] | AFR55550.1 |
| UL55 (gB) | envelope glycoprotein B [Human herpesvirus 5] | AKI19648.1 |
| UL55 (gB) | envelope glycoprotein B [Human herpesvirus 5] | AGL96655.1 |
| UL55 (gB) | glycoprotein B [Human herpesvirus 5] | AAA45932.1 |
| UL55 (gB) | glycoprotein B [Human herpesvirus 5] | AAA45933.1 |
| UL55 (gB) | glycoprotein B [Gorilla gorilla cytomegalovirus 2.1] | ACT68391.2 |
| UL55 (gB) | glycoprotein B [Human herpesvirus 5] | AAA45931.1 |
| UL55 (gB) | glycoprotein B [Human herpesvirus 5] | AAA45923.1 |
| UL55 (gB) | glycoprotein gB precursor [synthetic construct] | AAT68257.1 |
| UL55 (gB) | glycoprotein B [Human herpesvirus 5] | AAA45924.1 |
| UL55 (gB) | glycoprotein B [Human herpesvirus 5] | AAA45935.1 |
| UL73 (gN) | structural glycoprotein gpUL73 [Human herpesvirus 5] | AAM82374.1 |
| UL73 (gN) | structural glycoprotein gpUL73 [Human herpesvirus 5] | AAG23509.1 |
| UL73 (gN) | structural glycoprotein gpUL73 [Human herpesvirus 5] | AAM82416.1 |
| UL73 (gN) | envelope glycoprotein gpUL73 [Human herpesvirus 5] | AAO24877.1 |
| UL73 (gN) | envelope glycoprotein gpUL73 [Human herpesvirus 5] | ADE20136.1 |
| UL73 (gN) | envelope glycoprotein gpUL73 [Human herpesvirus 5] | YP_081521.1 |
| UL73 (gN) | glycoprotein N [Human herpesvirus 5] | ACI45834.1 |
| UL73 (gN) | envelope glycoprotein gpUL73 [Human herpesvirus 5] | AAO27562.1 |
| UL73 (gN) | glycoprotein N [Human herpesvirus 5] | ACI45816.1 |
| UL73 (gN) | envelope glycoprotein N [Human herpesvirus 5] | ACS93313.1 |
| UL73 (gN) | envelope glycoprotein N [Human herpesvirus 5] | AKI07618.1 |
| UL73 (gN) | glycoprotein N [Human herpesvirus 5] | ADC32373.1 |
| UL73 (gN) | structural glycoprotein gpUL73 [Human herpesvirus 5] | AAG23521.1 |
| UL73 (gN) | glycoprotein N [Human herpesvirus 5] | ADH42929.1 |
| UL73 (gN) | glycoprotein N [Human herpesvirus 5] | ADC32376.1 |
| UL73 (gN) | glycoprotein N [Human herpesvirus 5] | ADH42919.1 |
| UL73 (gN) | glycoprotein N [Human herpesvirus 5] | ACI45808.1 |
| UL73 (gN) | envelope glycoprotein gpUL73 [Human herpesvirus 5] | AAO24851.1 |
| UL73 (gN) | glycoprotein N [Human herpesvirus 5] | ABY48941.1 |
| UL73 (gN) | structural glycoprotein gpUL73 [Human herpesvirus 5] | AAG23512.1 |
| UL73 (gN) | structural glycoprotein gpUL73 [Human herpesvirus 5] | AAM82399.1 |
| UL73 (gN) | UL73 [Human herpesvirus 5] | ABZ04151.1 |
| UL73 (gN) | envelope glycoprotein gpUL73 [Human herpesvirus 5] | AAO24895.1 |
| UL73 (gN) | structural glycoprotein gpUL73 [Human herpesvirus 5] | AAM82420.1 |
| UL73 (gN) | structural glycoprotein gpUL73 [Human herpesvirus 5] | AAL77782.1 |
| UL73 (gN) | glycoprotein N [Human herpesvirus 5] | ADH42921.1 |
| UL73 (gN) | structural glycoprotein gpUL73 [Human herpesvirus 5] | AAM82396.1 |
| UL73 (gN) | envelope glycoprotein gpUL73 [Human herpesvirus 5] | AAO24881.1 |
| UL73 (gN) | envelope glycoprotein gpUL73 [Human herpesvirus 5] | AAO24889.1 |
| UL73 (gN) | envelope glycoprotein gpUL73 [Human herpesvirus 5] | AAO24892.1 |
| UL73 (gN) | glycoprotein N [Human herpesvirus 5] | ABY48942.1 |
| UL73 (gN) | glycoprotein N [Human herpesvirus 5] | ACI45800.1 |
| UL73 (gN) | envelope glycoprotein gpUL73 [Human herpesvirus 5] | AAO27565.1 |
| UL73 (gN) | glycoprotein N [Human herpesvirus 5] | ABY48936.1 |
| UL73 (gN) | glycoprotein N [Human herpesvirus 5] | ABY48935.1 |
| UL73 (gN) | structural glycoprotein gpUL73 [Human herpesvirus 5] | AAM82375.1 |
| UL73 (gN) | structural glycoprotein gpUL73 [Human herpesvirus 5] | AAM82403.1 |
| UL73 (gN) | envelope glycoprotein gpUL73 [Human herpesvirus 5] | AHB19542.1 |
| UL73 (gN) | envelope glycoprotein gpUL73 [Human herpesvirus 5] | AKI23166.1 |
| UL73 (gN) | structural glycoprotein gpUL73 [Human herpesvirus 5] | AAM82412.1 |
| UL73 (gN) | envelope glycoprotein gpUL73 [Human herpesvirus 5] | AAO24836.1 |
| UL73 (gN) | UL73 [Human herpesvirus 5] | ABZ04148.1 |
| UL73 (gN) | envelope glycoprotein gpUL73 [Human herpesvirus 5] | ACS93153.1 |
| UL73 (gN) | envelope glycoprotein gpUL73 [Human herpesvirus 5] | AGT36363.1 |
| UL73 (gN) | structural glycoprotein gpUL73 [Human herpesvirus 5] | AAG23511.1 |
| UL73 (gN) | glycoprotein N [Human herpesvirus 5] | ADH42925.1 |
| UL73 (gN) | glycoprotein N [Human herpesvirus 5] | ACI45830.1 |
| UL73 (gN) | structural glycoprotein gpUL73 [Human herpesvirus 5] | AAG23510.1 |
| UL73 (gN) | glycoprotein N [Human herpesvirus 5] | ACI45798.1 |
| UL73 (gN) | structural glycoprotein gpUL73 [Human herpesvirus 5] | AAL77762.1 |
| UL73 (gN) | glycoprotein N [Human herpesvirus 5] | ADH42926.1 |
| UL73 (gN) | structural glycoprotein gpUL73 [Human herpesvirus 5] | AAL77766.1 |
| UL73 (gN) | glycoprotein N [Human herpesvirus 5] | ACI45823.1 |
| UL73 (gN) | structural glycoprotein gpUL73 [Human herpesvirus 5] | AAM82378.1 |
| UL73 (gN) | structural glycoprotein gpUL73 [Human herpesvirus 5] | AAM82379.1 |
| UL73 (gN) | UL73 [Human herpesvirus 5] | ABZ04149.1 |
| UL73 (gN) | structural glycoprotein gpUL73 [Human herpesvirus 5] | AAL77764.1 |
| UL73 (gN) | glycoprotein N [Human herpesvirus 5] | ACI45835.1 |
| UL73 (gN) | glycoprotein N [Human herpesvirus 5] | ACI45825.1 |
| UL73 (gN) | glycoprotein N [Human herpesvirus 5] | ADH42931.1 |
| UL73 (gN) | envelope glycoprotein N [Human herpesvirus 5] | ACS93218.1 |
| UL73 (gN) | structural glycoprotein gpUL73 [Human herpesvirus 5] | AAM82408.1 |
| UL73 (gN) | glycoprotein N [Human herpesvirus 5] | ACI45831.1 |
| UL73 (gN) | envelope glycoprotein N [Human herpesvirus 5] | AAO27561.1 |
| UL73 (gN) | glycoprotein N [Human herpesvirus 5] | ACI45826.1 |
| UL74 (gO) | UL74 protein [Human herpesvirus 5] | AAN40064.1 |
| UL74 (gO) | envelope glycoprotein O [Human herpesvirus 5] | AKI16316.1 |
| UL74 (gO) | envelope glycoprotein O [Human herpesvirus 5] | ACS93259.1 |
| UL74 (gO) | UL74 protein [Human herpesvirus 5] | AAN40079.1 |
| UL74 (gO) | envelope glycoprotein O [Human herpesvirus 5] | AKI18316.1 |
| UL74 (gO) | glycoprotein O [Human herpesvirus 5] | ABY48961.1 |
| UL74 (gO) | glycoprotein O [Human herpesvirus 5] | ABY48960.1 |
| UL74 (gO) | glycoprotein O [Human herpesvirus 5] | ABY48959.1 |
| UL74 (gO) | envelope glycoprotein O [Human herpesvirus 5] | ACS93169.1 |
| UL74 (gO) | UL74 protein [Human herpesvirus 5] | AAN40044.1 |
| UL74 (gO) | envelope glycoprotein O [Human herpesvirus 5] | ACS93340.1 |
| UL74 (gO) | RecName: Full = Glycoprotein O; Short = gO; Flags: Precursor | P16750.1 |
| UL74 (gO) | UL74 protein [Human herpesvirus 5] | AAN40046.1 |
| UL74 (gO) | UL74 protein [Human herpesvirus 5] | AAN40054.1 |
| UL74 (gO) | envelope glycoprotein O [Human herpesvirus 5] | AKI08959.1 |
| UL74 (gO) | envelope glycoprotein O [Human herpesvirus 5] | AKI20327.1 |
| UL74 (gO) | UL74 protein [Human herpesvirus 5] | AAN40071.1 |
| UL74 (gO) | envelope glycoprotein O [Human herpesvirus 5] | AHB19710.1 |
| UL74 (gO) | envelope glycoprotein O [Human herpesvirus 5] | AKI07787.1 |
| UL74 (gO) | UL74 protein [Human herpesvirus 5] | AAN40043.1 |
| UL74 (gO) | UL74 protein [Human herpesvirus 5] | AAN40078.1 |
| UL74 (gO) | envelope glycoprotein O [Human herpesvirus 5] | ACS93309.1 |
| UL74 (gO) | envelope glycoprotein O [Human herpesvirus 5] | ACS93234.1 |
| UL74 (gO) | UL74 protein [Human herpesvirus 5] | AAN40040.1 |
| UL74 (gO) | envelope glycoprotein O [Human herpesvirus 5] | AKI19491.1 |
| UL74 (gO) | envelope glycoprotein O [Human herpesvirus 5] | AKI16979.1 |
| UL74 (gO) | envelope glycoprotein O [Human herpesvirus 5] | AKI20998.1 |
| UL74 (gO) | envelope glycoprotein O [Human herpesvirus 5] | AKI23000.1 |
| UL74 (gO) | envelope glycoprotein O [Human herpesvirus 5] | AKI10806.1 |
| UL74 (gO) | UL74 protein [Human herpesvirus 5] | AAN40073.1 |
| UL74 (gO) | UL74 protein [Human herpesvirus 5] | AAN40057.1 |
| UL74 (gO) | UL74 protein [Human herpesvirus 5] | AAN40050.1 |
| UL74 (gO) | envelope glycoprotein O [Human herpesvirus 5] | ABY48952.1 |
| UL74 (gO) | envelope glycoprotein O [Human herpesvirus 5] | AKI09296.1 |
| UL74 (gO) | envelope glycoprotein O [Human herpesvirus 5] | ACS93149.1 |
| UL74 (gO) | envelope glycoprotein O [Human herpesvirus 5] | AKI14979.1 |
| UL74 (gO) | UL74 protein [Human herpesvirus 5] | AAN40060.1 |
| UL74 (gO) | envelope glycoprotein O [Human herpesvirus 5] | ABY48954.1 |
| UL74 (gO) | envelope glycoprotein O [Human herpesvirus 5] | ABY48955.1 |
| UL74 (gO) | envelope glycoprotein O [Human herpesvirus 5] | ACS93219.1 |
| UL74 (gO) | envelope glycoprotein O [Human herpesvirus 5] | ACS93164.1 |
| UL74 (gO) | envelope glycoprotein O [Human herpesvirus 5] | YP_081522.1 |
| UL74 (gO) | envelope glycoprotein O [Human herpesvirus 5] | ABY48956.1 |
| UL74 (gO) | envelope glycoprotein O [Human herpesvirus 5] | AKI11474.1 |
| UL74 (gO) | UL74 protein [Human herpesvirus 5] | AAN40039.1 |
| UL74 (gO) | UL74 protein [Human herpesvirus 5] | AAN40041.1 |
| UL74 (gO) | envelope glycoprotein O [Human herpesvirus 5] | ACS93154.1 |
| UL74 (gO) | envelope glycoprotein O [Human herpesvirus 5] | ACT81745.1 |
| UL74 (gO) | envelope glycoprotein O [Human herpesvirus 5] | ACS92164.1 |
| UL74 (gO) | UL74 protein [Human herpesvirus 5] | AAN40052.1 |
| UL74 (gO) | envelope glycoprotein O [Human herpesvirus 5] | AHJ86161.1 |
| UL74 (gO) | envelope glycoprotein O [Human herpesvirus 5] | ACS93204.1 |
| UL74 (gO) | envelope glycoprotein O [Human herpesvirus 5] | AKI15314.1 |
| UL74 (gO) | envelope glycoprotein O [Human herpesvirus 5] | ACZ80315.1 |
| UL74 (gO) | envelope glycoprotein O [Human herpesvirus 5] | AKI23332.1 |
| UL74 (gO) | envelope glycoprotein O [Human herpesvirus 5] | ACU83724.1 |
| UL74 (gO) | UL74 protein [Human herpesvirus 5] | AAN40047.1 |
| UL74 (gO) | envelope glycoprotein O [Human herpesvirus 5] | AHV84021.1 |
| UL74 (gO) | UL74 protein [Human herpesvirus 5] | AAN40056.1 |
| UL74 (gO) | envelope glycoprotein O [Human herpesvirus 5] | AKI22164.1 |
| UL74 (gO) | envelope glycoprotein O [Human herpesvirus 5] | ACS93189.1 |
| UL74 (gO) | UL74 protein [Human herpesvirus 5] | AAN40074.1 |
| UL74 (gO) | envelope glycoprotein O [Human herpesvirus 5] | AKI18820.1 |
| UL74 (gO) | envelope glycoprotein O [Human herpesvirus 5] | AKI07619.1 |
| UL74 (gO) | UL74 protein [Human herpesvirus 5] | AAN40072.1 |
| UL74 (gO) | envelope glycoprotein O [Human herpesvirus 5] | AKI19991.1 |
| UL74 (gO) | UL74 protein [Human herpesvirus 5] | AAN40062.1 |
| UL74 (gO) | envelope glycoprotein O [Human herpesvirus 5] | AKI10471.1 |
| UL74 (gO) | UL74 protein [Human herpesvirus 5] | AAN40042.1 |
| UL74 (gO) | envelope glycoprotein O [Human herpesvirus 5] | AAT91377.1 |
| UL74 (gO) | envelope glycoprotein O [Human herpesvirus 5] | ACI45857.1 |
| UL74 (gO) | UL74 protein [Human herpesvirus 5] | AAP88253.1 |
| | RecName: Full = Large structural phosphoprotein: | P08318.1 |
| | AltName: Full = 150 kDa matrix phosphoprotein; | |
| | AltName: Full = 150 kDa phosphoprotein; | |
| | Short = pp150; AltName: FulHBasic phosphoprotein; | |
| | Short = BPP; AltName: Full = Phosphoprotein UL32; | |
| | AltName: Full = Tegument protein UL32 | |
| | UL32 [Human herpesvirus 5] | ABV71562.1 |
| | UL32 [Human herpesvirus 5] | AAG31644.1 |
| | UL32 [Human herpesvirus 5] | AAS48942.1 |
| | UL83 [Human herpesvirus 5] | ABV71605.1 |
| | RecName:Full = 65 kDa phosphoprotein; Short = pp65; | P06725.2 |
| | AltName: Full = 65 kDa matrix phosphoprotein; | |
| | AltName: Full = Phosphoprotein UL83; AltName: | |
| | Full = Tegument protein UL83 | |
| | RecName: Full = 65 kDa phosphoprotein; Short = pp65; | P18139.2 |
| | AltName: Full = 64 kDa matrix phosphoprotein; | |
| | Short = pp64; AltName: Full = GP64; AltName: Full = | |
| | Phosphoprotein UL83; AllName: Full-Tegument protein | |
| | UL83 [Human herpesvirus 5 strain Towne] | |
| | HCMVUL115 [Human herpesvirus 5] | CAA35317.1 |
| | truncated UL115 protein [Human herpesvirus 5] | ADG34192.1 |

### Example 22: Expression of mRNA Vaccine Constructs Encoding the hCMV Pentameric Complex in HeLa Cells

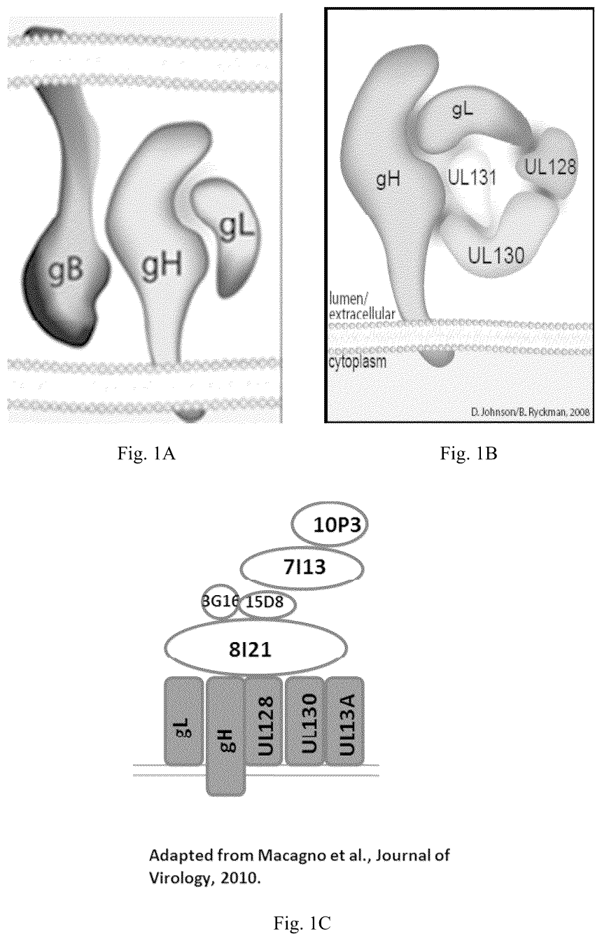

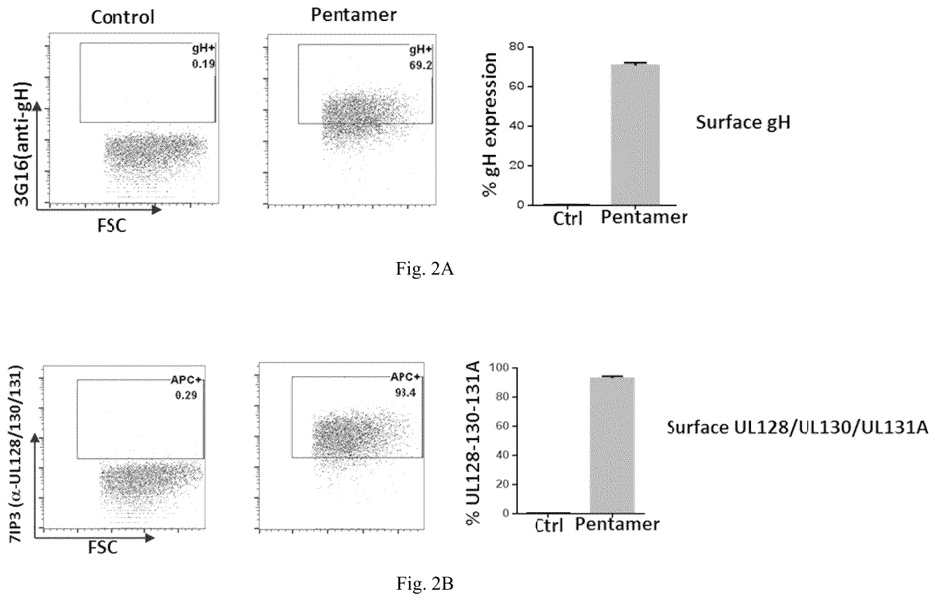

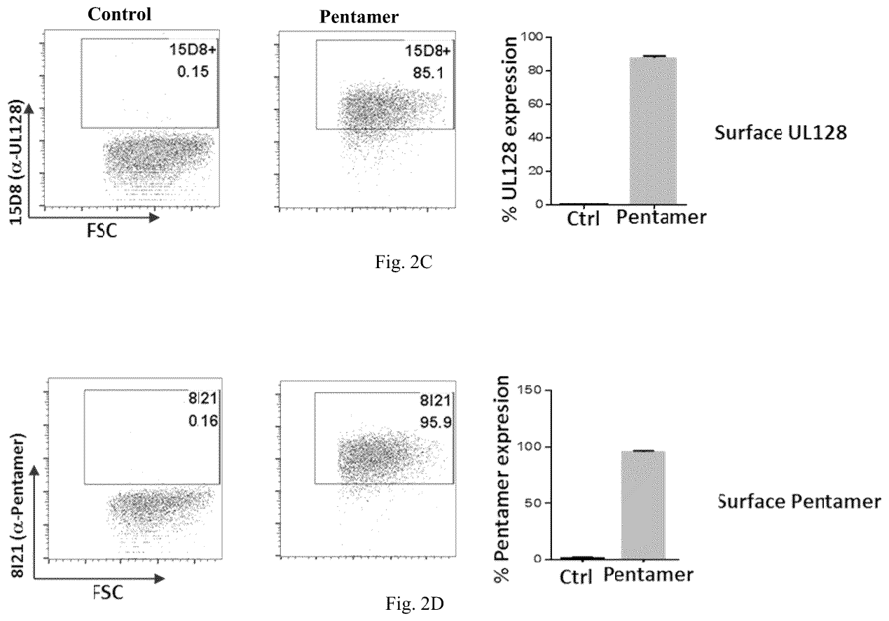

Expression of mRNA vaccine constructs encoding the subunits of the hCMV pentameric complex, including gH, gL, UL 128, UL130, and UL 131A was tested (FIG. 1B). mRNAs encoding each subunit were mixed at a gH:gL:UL128:UL130:UL131A ratio of 4:2:1:1:1. The total amount of mRNA used for transfecting HeLa cells was 2 μg. The transfected HeLa cells were incubated for 24 hours before they were analyzed using fluorescence-activated cell sorting (FACS) on a flow cytometer for the surface expression of the pentameric complex subunits as well as the complete pentamer (FIGS. 2A-2D). Antibodies specific for gH, UL128, the UL128/130/131A complex, or the complete pentamer were used for the detection of surface expression of the proteins. Surface expression of gH, UL128, the UL128/130/131A complex, and the complete pentameric complex were detected in HeLa cells (FIGS. 2A-2D).

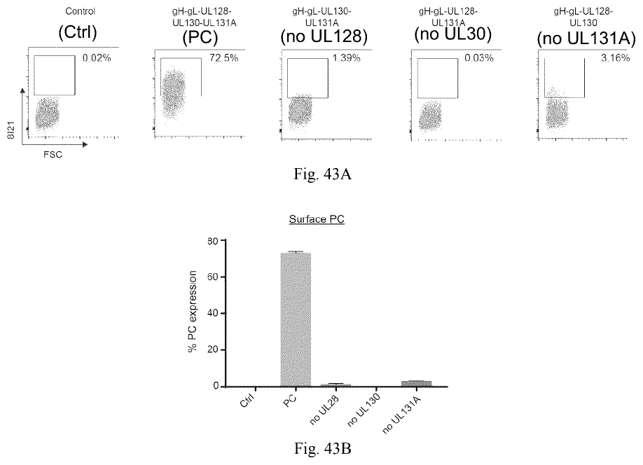

Similar results were observed when the pentameric components were transfected at equal mass ratios (FIGS. 43A-43B).

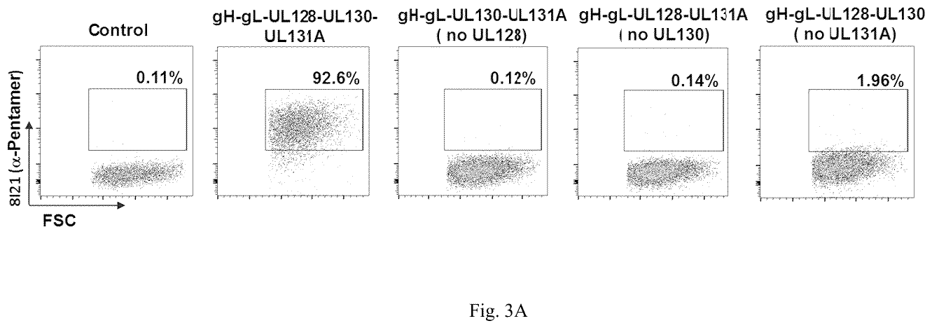

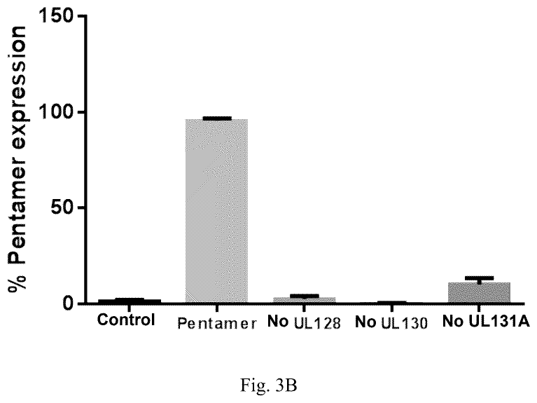

Different combinations of the mRNAs encoding the pentameric subunits were also tested to determine whether all of the core subunits were need for the surface expression of the complete pentameric complex (FIGS. 3A-3B). The experiments were carried out as described above with the indicated mRNA combinations. An antibody specific for the complete pentameric complex was used (8121). The results show that the pentameric complex does not express on the cell surface in the absence of any of UL128, UL130, or UL 31A.

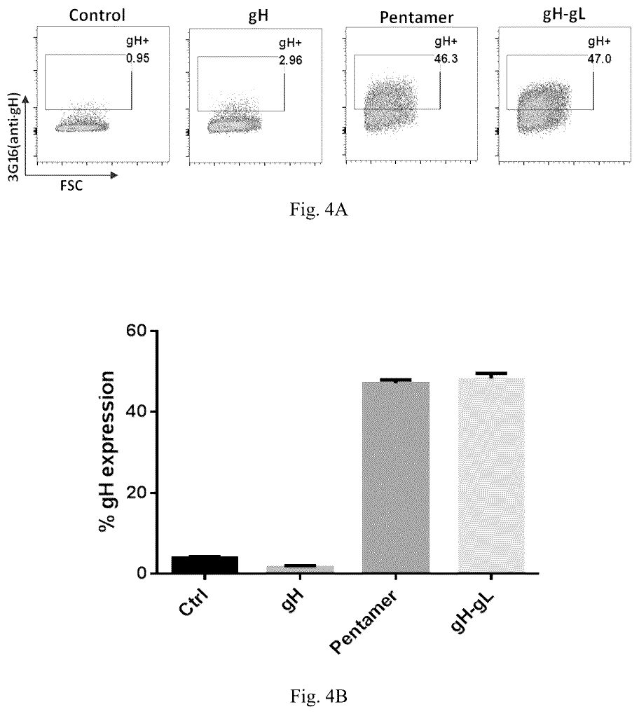

Next, the surface expression of the gH glycoprotein with or without gL was tested. The experiments were carried out as described above using mRNA constructs encoding gH, gH and gL, or constructs encoding the pentameric complex. An antibody specific for gH (3G16) was used. The results showed that expression of gH alone does not lead to gH expression on the cell surface. However, when gH was complexed with gL, a similar level of gH was detected on the surface of the HeLa cells as when all subunits in the pentameric complex were expressed (FIGS. 4A-4B).

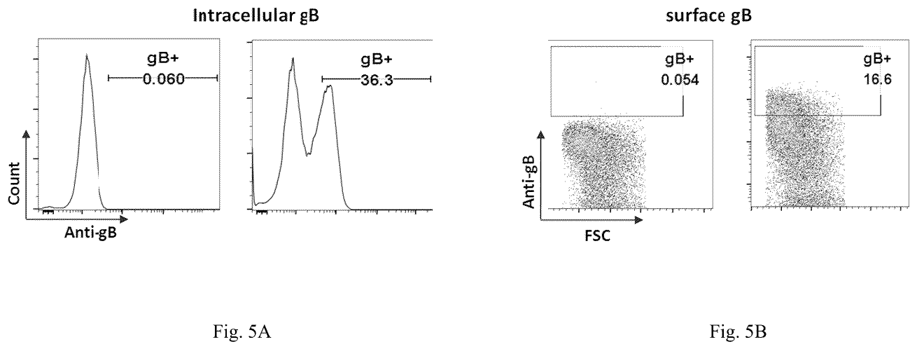

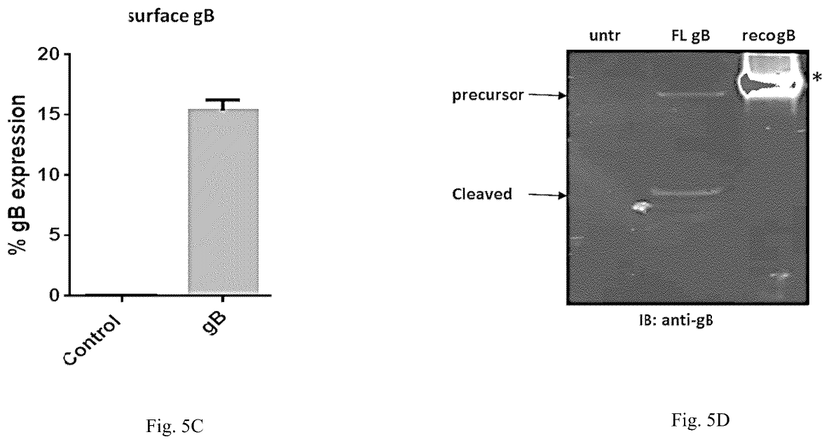

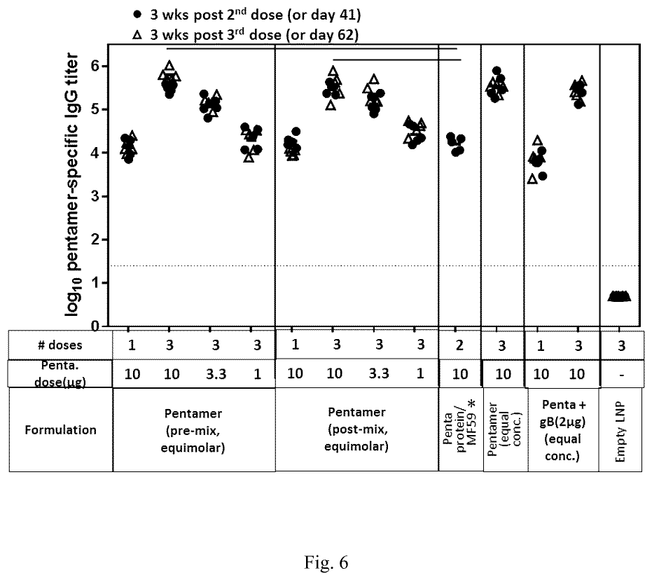

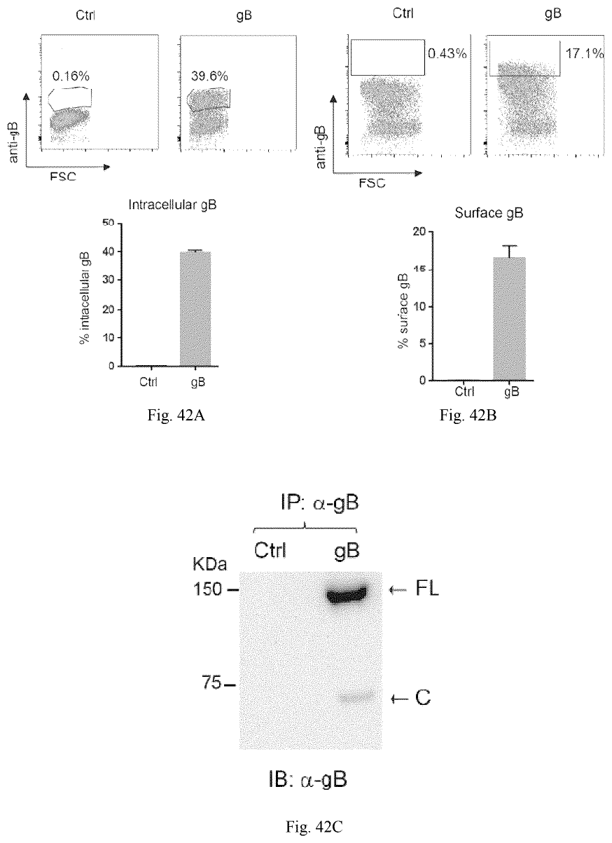

The intracellular and surface expression of gB was also tested using antibodies specific for gB. FIGS. 5A and 42A show intracellular gB expression. The surface expression of gB was measured by FACS on a flow cytometer and surface expression of gB was detected (FIGS. 5B and 42B). The quantification of gB surface expression is shown in FIGS. 5C, 42A and 42B. Further, an immunoblot conducted on cell lysates from HeLa cells transfected with mRNA constructs encoding gB is shown in FIGS. 5D and 42C. Untransfected HeLa cell lysates were used as a negative control and reconstituted full-length gB protein was used as a positive control. As shown in FIG. 5D, middle lane, or FIG. 42C, right lane, both full-length gB (the precursor) and the mature gB after proteolytic cleavage were detected.

### Example 23: High Titers of Anti-Pentameric Antibodies Following Immunization with hCMV Pentameric Complex mRNA Vaccine Constructs

The immunogenicity of candidate hCMV mRNA vaccine constructs encoding the pentameric complex subunits and/or the gB antigen was tested in mice. The immunization schedule and mRNA formulations are shown in Table 4 below.

Mice were divided into groups (5 mice per group) and vaccinated on day 0, 21, and 42 via intramuscular (IM) routes. One group of mice was vaccinated with empty lipid nanoparticles (LNP) as a control. Other groups of mice received hCMV mRNA vaccine constructs encoding the pentameric complex, the gB antigen, both the pentameric complex and gB antigen, or either the pentameric protein complex or the gB protein antigen combined with MF59. When mRNA vaccine constructions were given, different preparation procedures were used. The “pre-mix” mRNAs were pre-mixed and then formulated, while the “post-mix” mRNAs were individually formulated and then mixed. The mRNAs encoding all the subunits of the pentameric complex were formulated with different ratios as shown in Table 4: gH-gL-UL128-UL130-UL131A was 4:2:1:1:1 or 1:1:1:1:1. gB+pentamer was formulated at 1:1:1:1:1:1. The dose schedules used are indicated in Table 4.

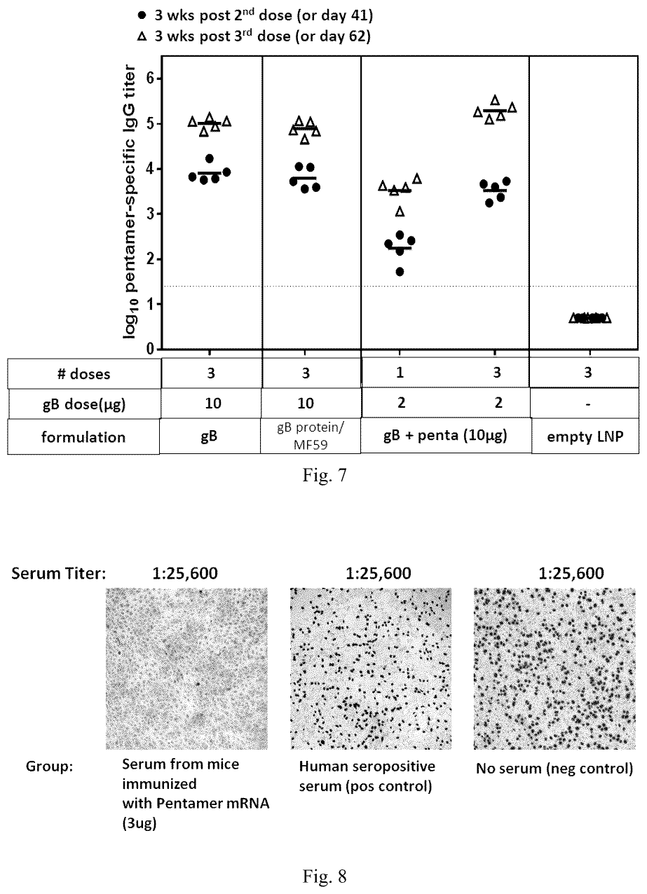

Mice sera were collected from each mouse on days −1 (pre-dos), 20, 41, 62, and 84. Individual bleeds from all time points were tested via ELISA assay carried out on plates coated with hCMV pentamers. Serum samples typically were diluted 1:100 for the assay. Incubation and washing protocols were performed using routine methods. Data was read at 450 nm wavelength. Data was reported and plotted (FIGS. 6 and 7). FIG. 6 shows that anti-pentamer-specific IgG were induced by hCMV mRNA vaccine constructs. However, little or no boosting was observed after the 3^{rd} immunization. IgG response was maintained from 6-9 weeks following a single immunization. Adding mRNAs encoding gB to mRNAs encoding the pentameric complex subunits did not interefere with anti-pentameric IgG production. Different molar ratios of the mRNAs encoding different pentameric complex subunits did not lead to different IgG induction levels. FIG. 7 shows that the mRNA vaccine constructs encoding gB induced anti-gB IgG response. IgG titers were similar for gB mRNA compared to gB protein/MF59 at 10 μg dose after three immunizations. A boost response was observed after the 3^{rd} immunization of gB mRNAs or antigens. Adding mRNAs encoding the pentameric complex subunits to mRNAs encoding gB did not interefere with anti-gB IgG production.

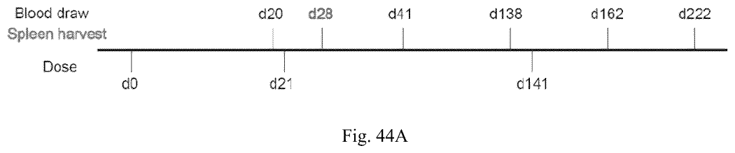

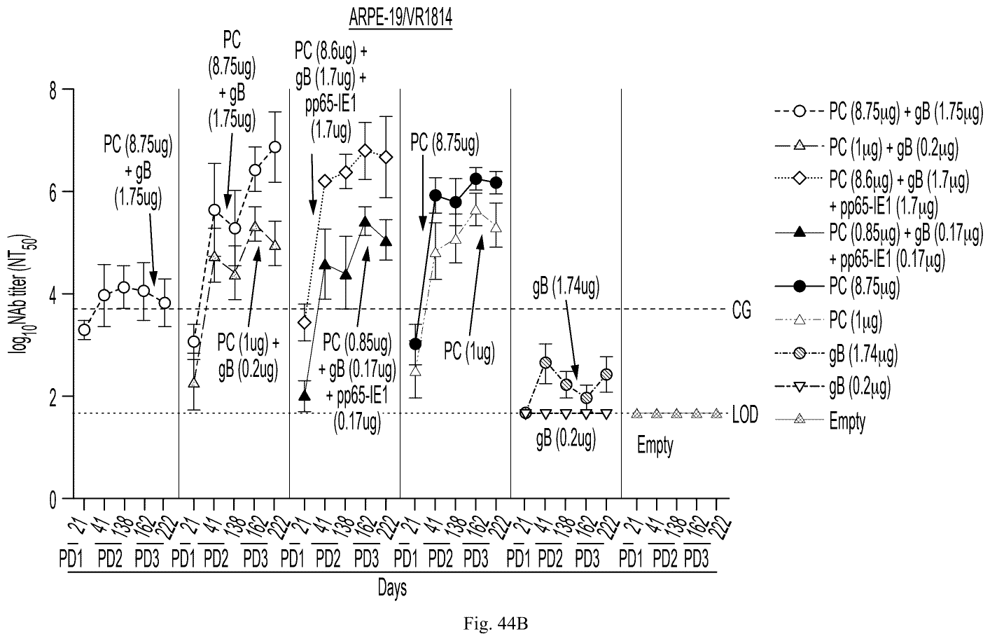

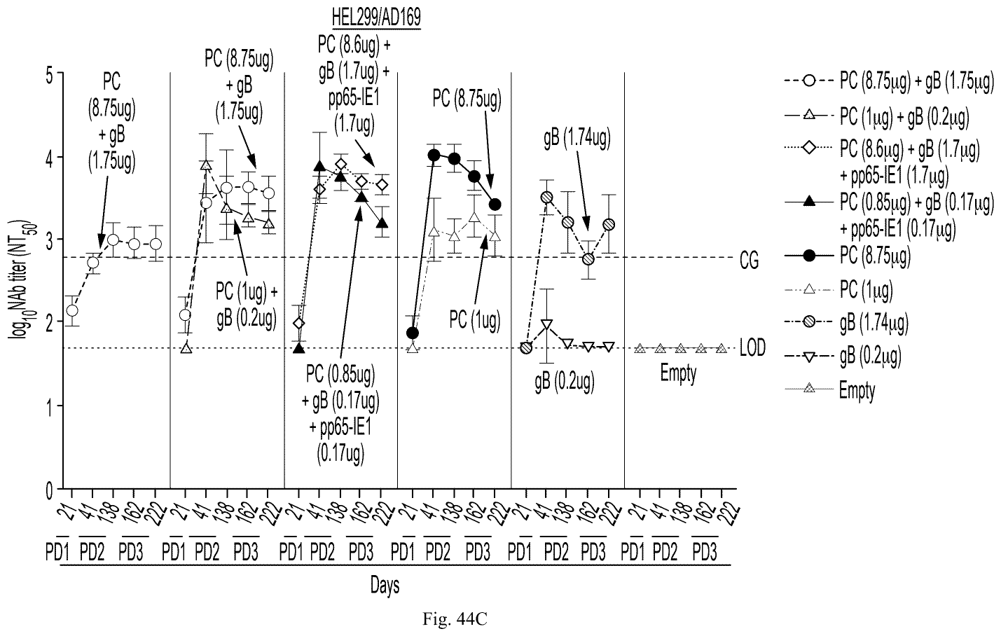

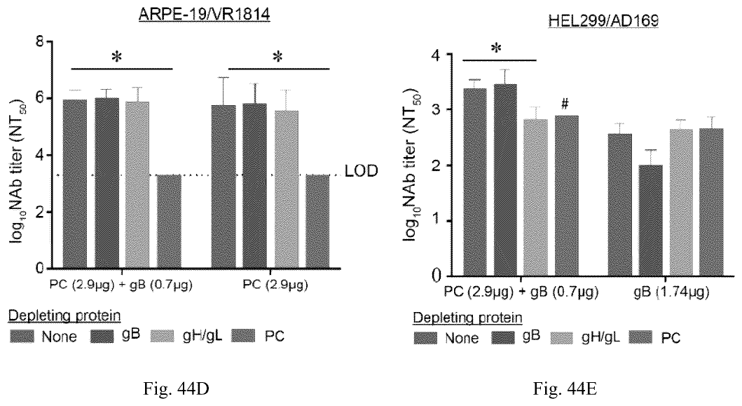

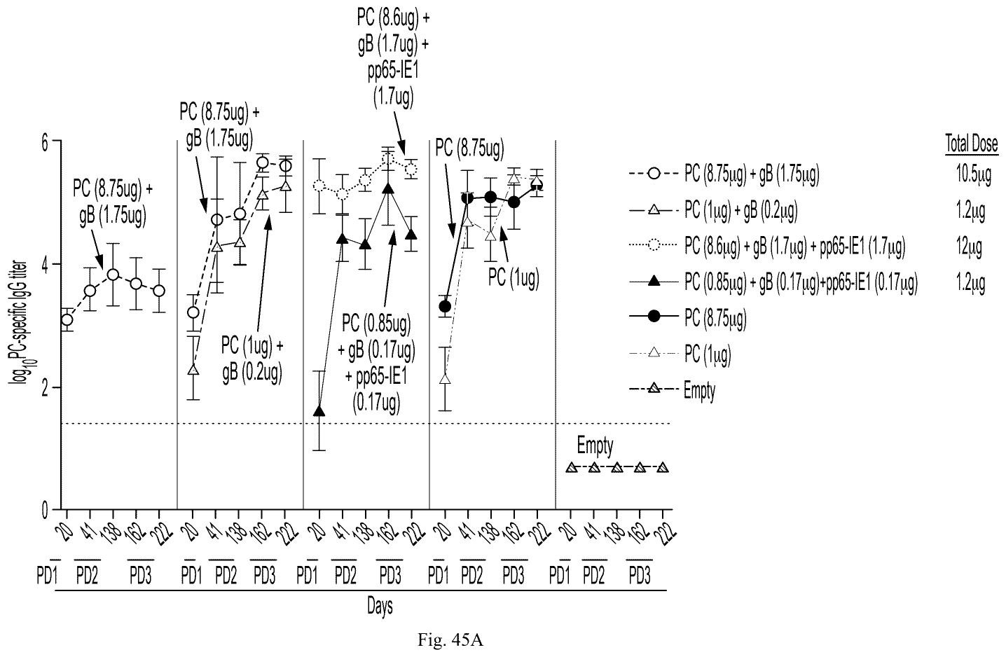

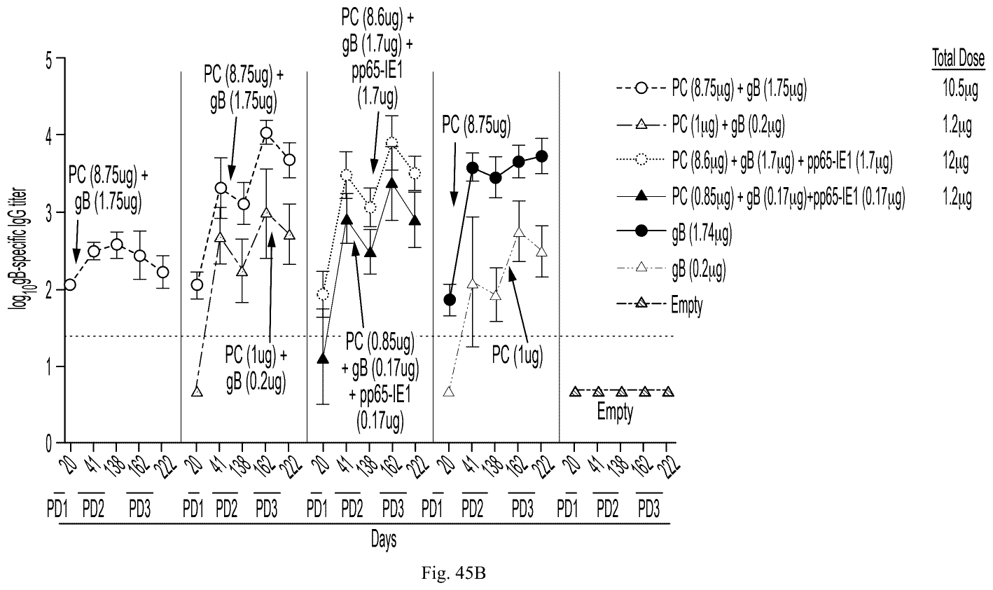

In one embodiment, mice were immunized using the regimen shown in FIG. 44A, and mice sera were collected according to the schedule in FIG. 44A. Antibody response to the pentameric complex (PC) and gB was evaluated by ELISA. Increases in antibody titers against both antigens were observed with increasing dose levels, which were boosted after a second or third dose of vaccine (FIGS. 45A-45B). Notably, the anti-PC and anti-gB antibody titers were not affected by the presence of the opposing antigen, indicating a lack of interference by combining two different antigens in the same LNP (FIGS. 45A-45B).

The ability of these antibodies to block CMV infection of epithelial and fibroblast cells in vitro was evaluated. Microneutralization assays showed potent and durable neutralizing antibodies against both cell types (FIGS. 44B and 44C). To evaluate the potency of the neutralizing antibodies elicited by CMV mRNA vaccine, Cytogam, which is used clinically for CMV infection prophylaxis, was used as a control. To enable direct comparison, Cytogam was diluted to approximate the maximum concentration in human sera after dosing. The results showed that the neutralizing antibody titers were higher than or similar to Cytogam for all vaccine groups tested at dose levels above 1 μg (FIGS. 44B and 44C).

To determine the specificity of antibodies that were generated with CMV mRNA vaccines, mouse immune sera were incubated with purified gB, gH/gL, or PC proteins prior to performing microneutralization assays on epithelial or fibroblast cells. The neutralization activity against epithelial cell infection was completely blocked by purified PC but not by the other human CMV antigens tested (FIG. 44D). In fibroblast cells, neutralization activity of sera from mice immunized with PC+gB was partially competed by gH/gL and PC proteins, but not by gB protein (FIG. 44E). This suggested that immunization with PC also generated anti-gH/gL antibodies and that the same neutralizing epitopes in gH/gL are also exposed when gH/gL is part of PC.

### Example 24: Immunization with hCMV Pentameric Complex mRNA Elicits Highly Potent Neutralizing Antibodies in Mice

Neutralization assays were conducted in epithelial cell line ARPE-19 infected with hCMV clinical isolate VR1814 were conducted. Mice were immunized according to the methods in Example 23. Mouse serum samples were collected 3 weeks after the second immunization (on day 41). Mice sera collected from mice immunized with 3 μg of hCMV mRNA pentameric vaccine constructs were diluted (1:25600) and added to the infected cells. The cells were stained for hCMV IE1 protein(as an indication of the presence of hCMV in the cells). Results showed that serum from mice immunized with 3 μg of hCMV pentameric mRNA vaccine constructs were able to neutralize the hCMV in ARPE-19 cells, while the controls of human seropositive serum or no serum did not neutralize the CMV in ARPE-19 cells (FIG. 8).

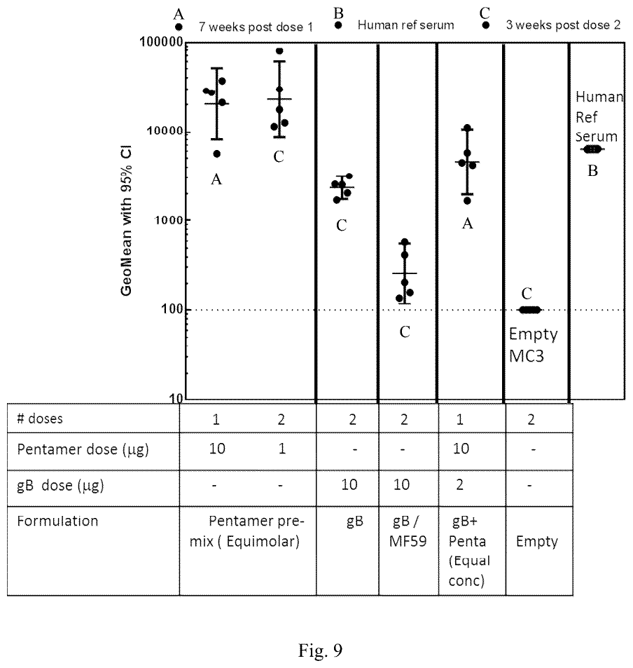

The hCMV neutralization titers of mouse serum measured in ARPE-19 cells infected with clinical hCMV isolate strain VR 1814 are shown in FIG. 9 and Table 5.

TABLE 4
Immunization and bleed schedule

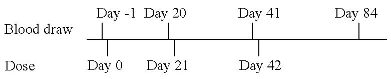

| Group | Vaccine | Route | Dose schedule | N |
|---|---|---|---|---|
| 1 | Pentamer (*4:2:1:1:1, premix), | IM | d 0 (10 ug) | 5 |
| | Equimolar | | | |
| 2 | Pentamer (4:2:1:1:1, pre-mix) | IM | d 0, d 21, d 42 | 5 |
| | | | (10 ug, 3 ug, | |
| | | | 1 ug) | |
| 3 | Pentamer (4:2:1:1:1), post-mix) | IM | d 0 (10 ug) | 5 |
| 4 | Pentamer (4:2:1:1:1, post-mix) | IM | d 0, d 21, d 42 | 5 |
| | | | (10 ug, 3 ug, | |
| | | | 1 ug) | |
| 5 | Pentamer (1:1:1:1:1, premix), | IM | d 0, d 21, d 42 | 5 |
| | Equal conc | | (10 ug) | |
| 6 | gB | IM | d 0, d 21, d 42 | 5 |
| | | | (10 ug) | |
| 7 | gB + Pentamer | IM | d 0 | 5 |
| | (1:1:1:1:1:1, pre-mix) | | (12 ug) | |
| 8 | gB + Pentamer | IM | d 0, d 21, d 42 | 5 |
| | (1:1:1:1:1:1, pre-mix) | | (12 ug) | |
| 9 | Pentamer protein/MF59 | IM | d 21, d 42 | 5 |
| | | | (10 ug) | |
| 10 | gB proein/MF59 | IM | d 0, d 21, d 42 | 5 |
| | | | (10 ug) | |
| 11 | Empty LNP | IM | d 0, d 21, d 42 | 5 |

TABLE 5
hCMV neutralization titers of mouse serum measured in
ARPE-19 cells infected with clinical isolate VR1814

| | # of | Dose | NT50 |
|---|---|---|---|
| Formulation | doses | (ug) | Titer |
| Pentamer, | 2 | 10 | >2E4 |
| Pre-mix | | | |
| (Equimoiar) | | | |
| Pentamer, | 2 | 3 | >2E4 |
| Pre-mix | | | |
| (Equimolar) | | | |
| Pentamer, | 2 | 10 | >2E4 |
| Post-mix | | | |
| (Equimolar) | | | |
| Pentamer, | 2 | 3 | >2E4 |
| Post-mix | | | |
| (Equimolar) | | | |
| Pentamer + gB | 2 | 12 | >2E4 |
| (Equal conc) | | | |

### Example 25: Second Generation hCMV Pentameric Complex mRNA Vaccine Constructs

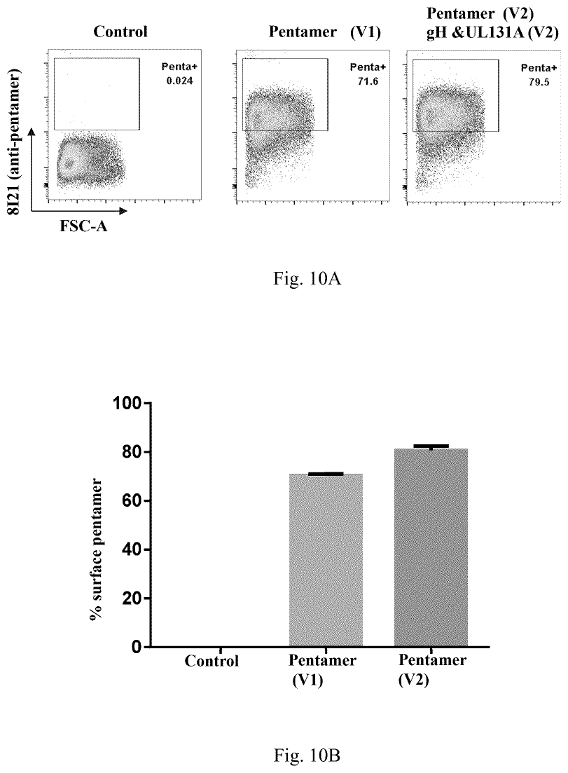

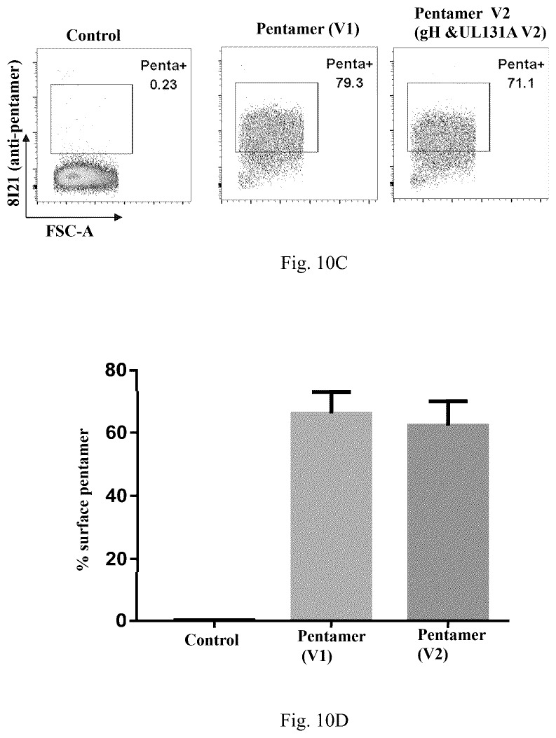

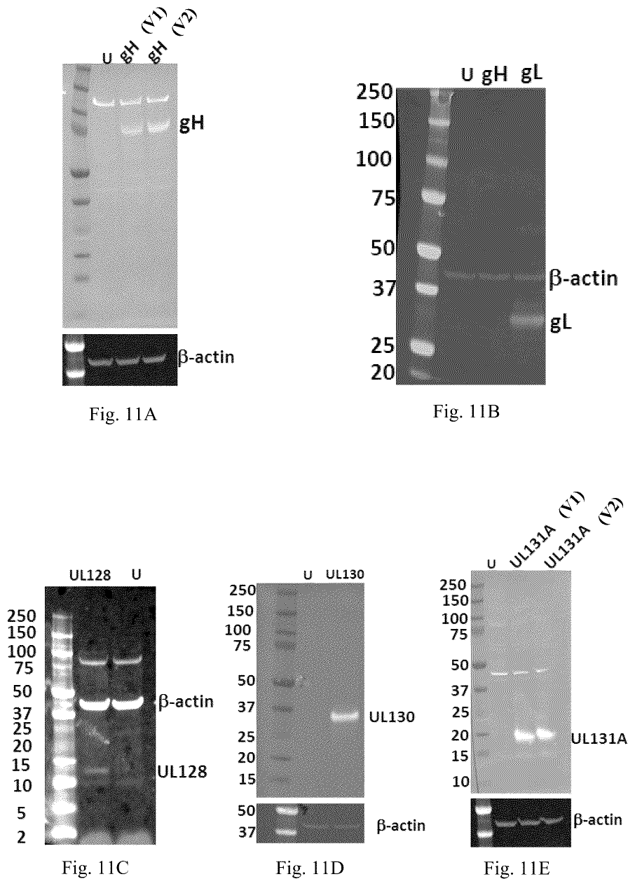

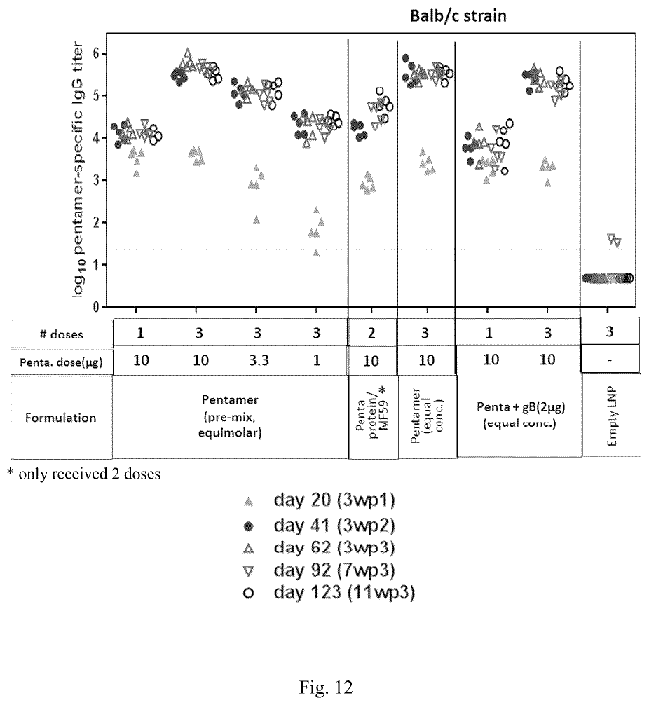

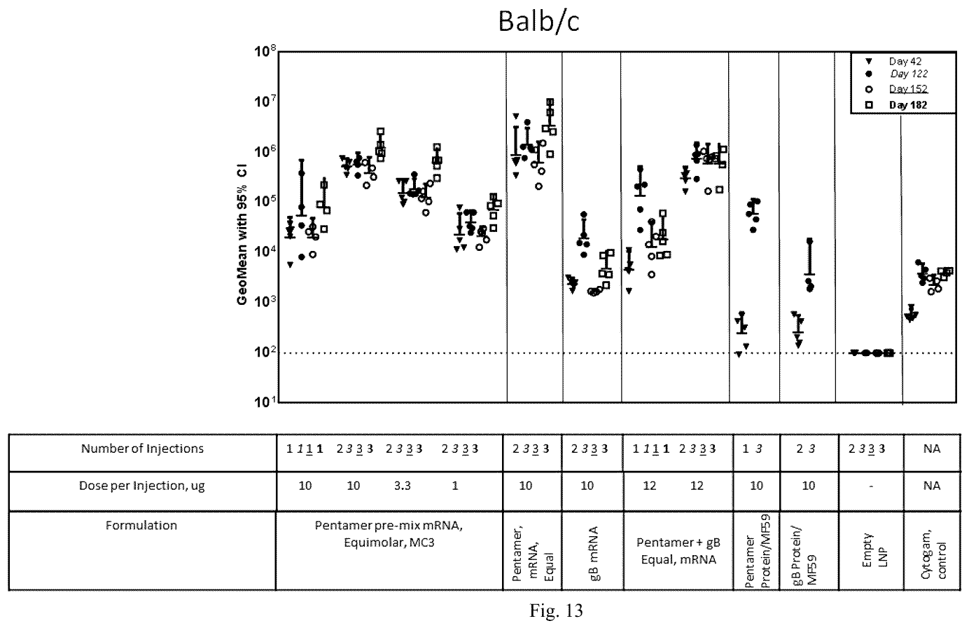

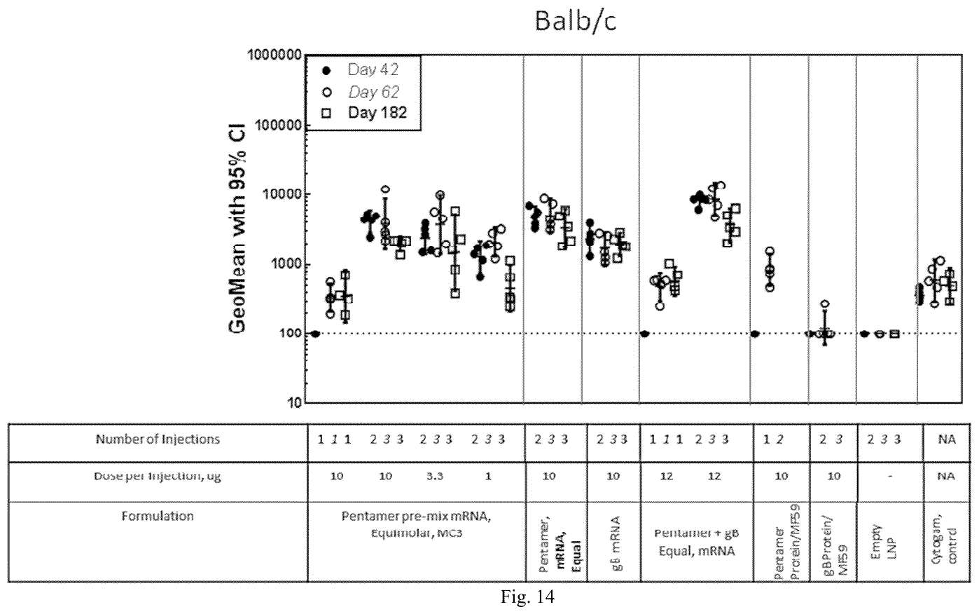

hCMV pentameric complex mRNA vaccine constructs were modified to produce second generation mRNA constructs. The nucleotide sequences of the second generation mRNA constructs and the encoded amino acid sequences are provided in Table 6. The expression of the second generation hCMV mRNA vaccine constructs was validated by western blot (FIGS. 11A-13E). Further, to test the surface expression of the hCMV pentamer using the second generation mRNA vaccine constructs. HeLa cells were transfected with 1.25 μg of each of the mRNA vaccine constructs (gH-gL-U1128-UL130-UL131A at 1:1:1:1:1). The transfected HeLa cells were then stained with pentamer-specific antibodies and analyzed with Fluorescence-activated cell sorting (FACS). The fluorescent cell population indicates surface expression of the hCMV pentamer (FIG. 10).

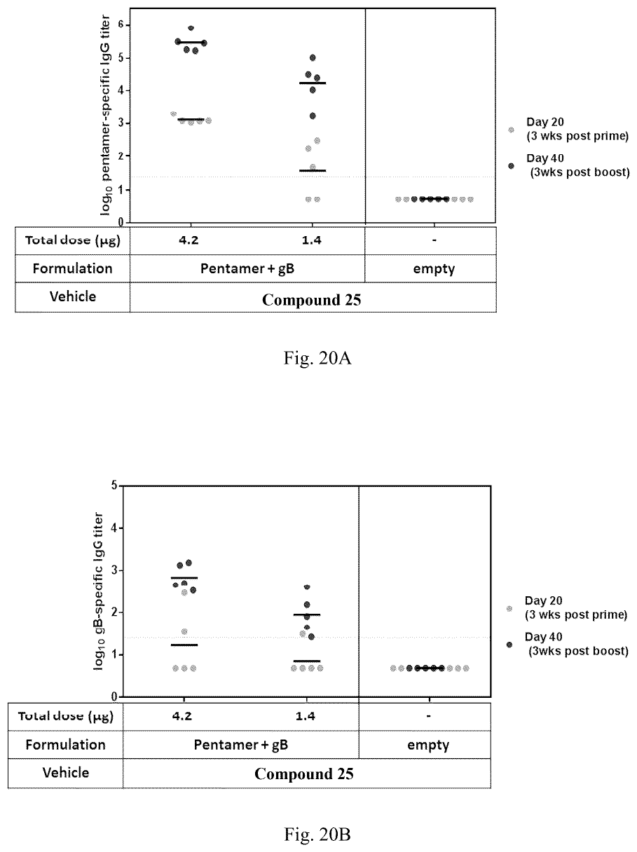

The second generation hCMV mRNA vaccines encoding the pentamer and gB were also formulated with Compound 25 lipids and the immunogenicity of the formulation was tested (FIGS. 20A-20B). Mice were immunized with a total dose of 4.2 μg or 1.4 μg of the mRNA vaccine. Mice serum samples were taken on day 20 and day 40 post immunization and the serum IgG titers were assessed on pentamer coated plates or gB coated plates. The second generation hCMV mRNA vaccines induced high levels of pentamer-specific (FIG. 20A) and gB-specific (FIG. 20B) antibodies.

An HCMV vaccine may comprise, for example, at least one RNA polynucleotide encoded by at least one of the following sequences or by at least one fragment or epitope of the following sequences:

Each of the sequences described herein encompasses a chemically modified sequence or an unmodified sequence which includes no nucleotide modifications.

The nucleotide sequences shown in Table 6 include open reading frame sequences linked to non-limiting examples of 5′ and 3′UTRs. It should be appreciated that the same open reading frames can also be linked to different 5′ and 3′ UTR sequences.

Examples of UTR sequences include:

5′ UTR coding sequence:
(SEQ ID NO: 145)
TCAAGCTTTTGGACCCTCGTACAGAAGCTAATACGACTCACTATAGGGA
AATAAGAGAGAAAAGAAGAGTAAGAAGAAATATAAGAGCCACC
5′ UTR (without promoter) coding sequence:
(SEQ ID NO: 146)
GGGAAATAAGAGAGAAAAGAAGAGTAAGAAGAAATATAAGAGCCACC
3′ UTR coding sequence:
(SEQ ID NO: 147)
TGATAATAGGCTGGAGCCTCGGTGGCCATGCTTCTTGCCCCTTGGGCCT
CCCCCCAGCCCCTCCTCCCCTTCCTGCACCCGTACCCCCGTGGTCTTTG
AATAAAGTCTGAGTGGGCGGC.

TABLE 6
Second Generation hCMV mRNA Vaccine Construct Sequences

| Name of | | |
|---|---|---|
| mRNA | ORF Sequence (5′ UTR sequence is bolded and 3′ UTR | SEQ ID |
| Construct | sequence is underlined) | NO |
| hCMV_gH | TCAAGCTTTTGGACCCTCGTACAGAAGCTAATACGACTCACTATAGGGAA | 58 |
| dimer, | ATAAGAGAGAAAAGAAGAGTAAGAAGAAATATAAGAGCCACCATGCGGCC | |
| nucleotide | AGGCCTCCCCTCCTACCTCATCATCCTCGCCGTCTGTCTCTTCAGCCACCTACTT | |
| sequence | TCGTCACGATATGGCGCAGAAGCCGTATCCGAACCGCTGGACAAAGCGTTTCA | |
| | CCTACTGCTCAACACCTACGGGAGACCCATCCGCTTCCTGCGTGAAAATACCA | |
| | CCCAGTGTACCTACAACAGCAGCCTCCGTAACAGCACGGTCGTCAGGGAAAAC | |
| | GCCATCAGTTTCAACTTCTTCCAAAGCTATAATCAATACTATGTATTCCATATG | |
| | CCTCGATGTCTCTTTGCGGGTCCTCTGGCGGAGCAGTTTCTGAACCAGGTAGAT | |
| | CTGACCGAAACCCTGGAAAGATACCAACAGAGACTTAACACTTACGCGCTGGT | |
| | ATCCAAAGACCTGGCCAGCTACCGATCTTTCTCGCAGCAGCTAAAGGCACAAG | |
| | ACAGCCTAGGTGAACAGCCCACCACTGTGCCACCGCCCATTGACCTGTCAATA | |
| | CCTCACGTTTGGATGCCACCGCAAACCACTCCACACGGCTGGACAGAATCACA | |
| | TACCACCTCAGGACTACACCGACCACACTTTAACCAGACCTGTATCCTCTTTGA | |
| | TGGACACGATCTACTATTCAGCACCGTCACACCTTGTTTGCACCAAGGCTTTTA | |
| | CCTCATCGACGAACTACGTTACGTTAAAATAACACTGACCGAGGACTTCTTCGT | |
| | AGTTACGGTGTCCATAGACGACGACACACCCATGCTGCTTATCTTCGGCCATCT | |
| | TCCACGCGTACTTTTCAAAGCGCCCTATCAACGCGACAACTTTATACTACGACA | |
| | AACTGAGAAACACGAGCTCCTGGTGCTAGTTAAGAAAGATCAACTGAACCGTC | |
| | ACTCTTATCTCAAAGACCCGGACTTTCTTGACGCCGCACTTGACTTCAACTACC | |
| | TAGACCTCAGCGCACTACTACGTAACAGCTTTCACCGTTACGCCGTGGATGTAC | |
| | TCAAGAGCGGTCGATGTCAGATGCTGGACCGCCGCACGGTAGAAATGGCCTTC | |
| | GCCTACGCATTAGCACTGTTCGCAGCAGCCCGACAAGAAGAGGCCGGCGCCCA | |
| | AGTCTCCGTCCCACGGGCCCTAGACCGCCAGGCCGCACTCTTACAAATACAAG | |
| | AATTTATGATCACCTGCCTCTCACAAACACCACCACGCACCACGTTGCTGCTGT | |
| | ATCCCACGGCCGTGGACCTGGCCAAACGAGCCCTTTGGACACCGAATCAGATC | |
| | ACCGACATCACCAGCCTCGTACGCCTGGTCTACATACTCTCTAAACAGAATCA | |
| | GCAACATCTCATCCCCCAATGGGCACTACGACAGATCGCCGACTTTGCCCTAA | |
| | AACTACACAAAACGCACCTGGCCTCTTTTCTTTCAGCCTTCGCACGCCAAGAAC | |
| | TCTACCTCATGGGCAGCCTCGTCCACTCCATGCTGGTACATACGACGGAGAGA | |
| | CGCGAAATCTTCATCGTAGAAACGGGCCTCTGTTCATTGGCCGAGCTATCACAC | |
| | TTTACGCAGTTGTTAGCTCATCCACACCACGAATACCTCAGCGACCTGTACACA | |
| | CCCTGTTCCAGTAGCGGGCGACGCGATCACTCGCTCGAACGCCTCACGCGTCTC | |
| | TTCCCCGATGCCACCGTCCCCGCTACCGTTCCCGCCGCCCTCTCCATCCTATCTA | |
| | CCATGCAACCAAGCACGCTGGAAACCTTCCCCGACCTGTTTTGCTTGCCGCTCG | |
| | GCGAATCCTTCTCCGCGCTGACCGTCTCCGAACACGTCAGTTATATCGTAACAA | |
| | ACCAGTACCTGATCAAAGGTATCTCCTACCCTGTCTCCACCACCGTCGTAGGCC | |
| | AGAGCCTCATCATCACCCAGACGGACAGTCAAACTAAATGCGAACTGACGCGC | |
| | AACATGCATACCACACACAGCATCACAGTGGCGCTCAACATTTCGCTAGAAAA | |
| | CTGCGCCTTTTGCCAAAGCGCCCTGCTAGAATACGACGACACGCAAGGCGTCA | |
| | TCAACATCATGTACATGCACGACTCGGACGACGTCCTTTTCGCCCTGGATCCCT | |
| | ACAACGAAGTGGTGGTCTCATCTCCGCGAACTCACTACCTCATGCTTTTGAAGA | |
| | ACGGTACGGTACTAGAAGTAACTGACGTCGTCGTGGACGCCACCGACAGTCGT | |
| | CTCCTCATGATGTCCGTCTACGCGCTATCGGCCATCATCGGCATCTATCTGCTC | |
| | TACCGCATGCTCAAGACATGCTGATAATAGGCTGGAGCCTCGGTGGCCATGCT | |
| | TCTTGCCCCTTGGGCCTCCCCCCAGCCCCTCCTCCCCTTCCTGCACCCGTACCCC | |
| | CGTGGTCTTTGAATAAAGTCTGAGTGGGCGGC | |
| hCMV_gH | UCAAGCUUUUGGACCCUCGUACAGAAGCUAAUACGACUCACUAUAGGGAAA | 189 |
| dimer, | UAAGAGAGAAAAGAAGAGUAAGAAGAAAUAUAAGAGCCACCAUGCGGCCAG | |
| nucleotide | GCCUCCCCUCCUACCUCAUCAUCCUCGCCGUCUGUCUCUUCAGCCACCUACU | |
| sequence | UUCGUCACGAUAUGGCGCAGAAGCCGUAUCCGAACCGCUGGACAAAGCGUU | |
| mRNA | UCACCUACUGCUCAACACCUACGGGAGACCCAUCCGCUUCCUGCGUGAAAAU | |
| | ACCACCCAGUGUACCUACAACAGCAGCCUCCGUAACAGCACGGUCGUCAGGG | |
| | AAAACGCCAUCAGUUUCAACUUCUUCCAAAGCUAUAAUCAAUACUAUGUAU | |
| | UCCAUAUGCCUCGAUGUCUCUUUGCGGGUCCUCUGGCGGAGCAGUUUCUGA | |
| | ACCAGGUAGAUCUGACCGAAACCCUGGAAAGAUACCAACAGAGACUUAACA | |
| | CUUACGCGCUGGUAUCCAAAGACCUGGCCAGCUACCGAUCUUUCUCGCAGCA | |
| | GCUAAAGGCACAAGACAGCCUAGGUGAACAGCCCACCACUGUGCCACCGCCC | |
| | AUUGACCUGUCAAUACCUCACGUUUGGAUGCCACCGCAAACCACUCCACACG | |
| | GCUGGACAGAAUCACAUACCACCUCAGGACUACACCGACCACACUUUAACCA | |
| | GACCUGUAUCCUCUUUGAUGGACACGAUCUACUAUUCAGCACCGUCACACCU | |
| | UGUUUGCACCAAGGCUUUUACCUCAUCGACGAACUACGUUACGUUAAAAUA | |
| | ACACUGACCGAGGACUUCUUCGUAGUUACGGUGUCCAUAGACGACGACACA | |
| | CCCAUGCUGCUUAUCUUCGGCCAUCUUCCACGCGUACUUUUCAAAGCGCCCU | |
| | AUCAACGCGACAACUUUAUACUACGACAAACUGAGAAACACGAGCUCCUGG | |
| | UGCUAGUUAAGAAAGAUCAACUGAACCGUCACUCUUAUCUCAAAGACCCGG | |
| | ACUUUCUUGACGCCGCACUUGACUUCAACUACCUAGACCUCAGCGCACUACU | |
| | ACGUAACAGCUUUCACCGUUACGCCGUGGAUGUACUCAAGAGCGGUCGAUG | |
| | UCAGAUGCUGGACCGCCGCACGGUAGAAAUGGCCUUCGCCUACGCAUUAGCA | |
| | CUGUUCGCAGCAGCCCGACAAGAAGAGGCCGGCGCCCAAGUCUCCGUCCCAC | |
| | GGGCCCUAGACCGCCAGGCCGCACUCUUACAAAUACAAGAAUUUAUGAUCAC | |
| | CUGCCUCUCACAAACACCACCACGCACCACGUUGCUGCUGUAUCCCACGGCC | |
| | GUGGACCUGGCCAAACGAGCCCUUUGGACACCGAAUCAGAUCACCGACAUCA | |
| | CCAGCCUCGUACGCCUGGUCUACAUACUCUCUAAACAGAAUCAGCAACAUCU | |
| | CAUCCCCCAAUGGGCACUACGACAGAUCGCCGACUUUGCCCUAAAACUACAC | |
| | AAAACGCACCUGGCCUCUUUUCUUUCAGCCUUCGCACGCCAAGAACUCUACC | |
| | UCAUGGGCAGCCUCGUCCACUCCAUGCUGGUACAUACGACGGAGAGACGCGA | |
| | AAUCUUCAUCGUAGAAACGGGCCUCUGUUCAUUGGCCGAGCUAUCACACUU | |
| | UACGCAGUUGUUAGCUCAUCCACACCACGAAUACCUCAGCGACCUGUACACA | |
| | CCCUGUUCCAGUAGCGGGCGACGCGAUCACUCGCUCGAACGCCUCACGCGUC | |
| | UCUUCCCCGAUGCCACCGUCCCCGCUACCGUUCCCGCCGCCCUCUCCAUCCUA | |
| | UCUACCAUGCAACCAAGCACGCUGGAAACCUUCCCCGACCUGUUUUGCUUGC | |
| | CGCUCGGCGAAUCCUUCUCCGCGCUGACCGUCUCCGAACACGUCAGUUAUAU | |
| | CGUAACAAACCAGUACCUGAUCAAAGGUAUCUCCUACCCUGUCUCCACCACC | |
| | GUCGUAGGCCAGAGCCUCAUCAUCACCCAGACGGACAGUCAAACUAAAUGCG | |
| | AACUGACGCGCAACAUGCAUACCACACACAGCAUCACAGUGGCGCUCAACAU | |
| | UUCGCUAGAAAACUGCGCCUUUUGCCAAAGCGCCCUGCUAGAAUACGACGAC | |
| | ACGCAAGGCGUCAUCAACAUCAUGUACAUGCACGACUCGGACGACGUCCUUU | |
| | UCGCCCUGGAUCCCUACAACGAAGUGGUGGUCUCAUCUCCGCGAACUCACUA | |
| | CCUCAUGCUUUUGAAGAACGGUACGGUACUAGAAGUAACUGACGUCGUCGU | |
| | GGACGCCACCGACAGUCGUCUCCUCAUGAUGUCCGUCUACGCGCUAUCGGCC | |
| | AUCAUCGGCAUCUAUCUGCUCUACCGCAUGCUCAAGACAUGCUGAUAAUAG | |
| | GCUGGAGCCUCGGUGGCCAUGCUUCUUGCCCCUUGGGCCUCCCCCCAGCCCC | |
| | UCCUCCCCUUCCUGCACCCGUACCCCCGUGGUCUUUGAAUAAAGUCUGAGUG | |
| | GGCGGC | |
| hCMV_gH | MRPGLPSYLIILAVCLFSHLLSSRYGAEAVSEPLDKAFHLLLNTYGRPIRFLRENTTQ | 59 |
| dimer, | CTYNSSLRNSTVVRENAISFNFFQSYNQYYVFHMPRCLFAGPLAEQFLNQVDLTET | |
| amino acid | LERYQQRLNTYALVSKDLASYRSFSQQLKAQDSLGEQPTTVPPPIDLSIPHVWMPP | |
| sequence | QTTPHGWTESHTTSGLHRPHFNQTCILFDGHDLLFSTVTPCLHQGFYLIDELRYVKI | |
| | TLTEDFFVVTVSIDDDTPMLLIFGHLPRVLFKAPYQRDNFILRQTEKHELLVLVKKD | |
| | QLNRHSYLKDPDFLDAALDFNYLDLSALLRNSFHRYAVDVLKSGRCQMLDRRTV | |
| | EMAFAYALALFAAARQEEAGAQVSVPRALDRQAALLQIQEFMITCLSQTPPRTTLL | |
| | LYPTAVDLAKRALWTPNQITDITSLVRLVYILSKQNQQHLIPQWALRQIADFALKL | |
| | HKTHLASFLSAFARQELYLMGSLVHSMLVHTTERREIFIVETGLCSLAELSHFTQLL | |
| | AHPHHEYLSDLYTPCSSSGRRDHSLERLTRLFPDATVPATVPAALSILSTMQPSTLE | |
| | TFPDLFCLPLGESFSALTVSEHVSYIVTNQYLIKGLSYPVSTTVVGQSLIITQTDSQTK | |
| | CELTRNMHTTHSITVALNISLENCAFCQSALLEYDDTQGVINIMYMHDSDDVLFAL | |
| | DPYNEVVVSSPRTHYLMLLKNGTVLEVTDVVVDATDSRLLMMSVYALSAIIGIYL | |
| | LYRMLKTC | |
| hCMV-gL, | TCAAGCTTTTGGACCCTCGTACAGAAGCTAATACGACTCACTATAGGGAA | 60 |
| nucleotide | ATAAGAGAGAAAAGAAGAGTAAGAAGAAATATAAGAGCCACCATGTGCCG | |
| sequence | CCGCCCGGATTGCGGCTTCTCTTTCTCACCTGGACCGGTGATACTGCTGTGGTG | |
| | TTGCCTTCTGCTGCCCATTGTTTCCTCAGCCGCCGTCAGCGTCGCTCCTACCGCC | |
| | GCCGAGAAAGTCCCCGCGGAGTGCCCCGAACTAACGCGCCGATGCTTGTTGGG | |
| | TGAGGTGTTTGAGGGTGACAAGTATGAAAGTTGGCTGCGCCCGTTGGTGAATG | |
| | TTACCGGGCGCGATGGCCCGCTATCGCAACTTATCCGTTACCGTCCCGTTACGC | |
| | CGGAGGCCGCCAACTCCGTGCTGTTGGACGAGGCTTTCCTGGACACTCTGGCC | |
| | CTGCTGTACAACAATCCGGATCAATTGCGGGCCCTGCTGACGCTGTTGAGCTCG | |
| | GACACAGCGCCGCGCTGGATGACGGTGATGCGCGGCTACAGCGAGTGCGGCG | |
| | ATGGCTCGCCGGCCGTGTACACGTGCGTGGACGACCTGTGCCGCGGCTACGAC | |
| | CTCACGCGACTGTCATACGGGCGCAGCATCTTCACGGAACACGTGTTAGGCTT | |
| | CGAGCTGGTGCCACCGTCTCTCTTTAACGTGGTGGTGGCCATACGCAACGAAG | |
| | CCACGCGTACCAACCGCGCCGTGCGTCTGCCCGTGAGCACCGCTGCCGCGCCC | |
| | GAGGGCATCACGCTCTTTTACGGCCTGTACAACGCAGTGAAGGAATTCTGCCT | |
| | GCGTCACCAGCTGGACCCGCCGCTGCTACGCCACCTAGATAAATACTACGCCG | |
| | GACTGCCGCCCGAGCTGAAGCAGACGCGCGTCAACCTGCCGGCTCACTCGCGC | |
| | TATGGCCCTCAAGCAGTGGATGCTCGCTGATAATAGGCTGGAGCCTCGGTGGC | |
| | CATGCTTCTTGCCCCTTGGGCCTCCCCCCAGCCCCTCCTCCCCTTCCTGCACCCG | |
| | TACCCCCGTGGTCTTTGAATAAAGTCTGAGTGGGCGGC | |
| hCMV-gL, | UCAAGCUUUUGGACCCUCGUACAGAAGCUAAUACGACUCACUAUAGGGAAA | 190 |
| nucleotide | UAAGAGAGAAAAGAAGAGUAAGAAGAAAUAUAAGAGCCACCAUGUGCCGCC | |
| sequence | GCCCGGAUUGCGGCUUCUCUUUCUCACCUGGACCGGUGAUACUGCUGUGGU | |
| mRNA | GUUGCCUUCUGCUGCCCAUUGUUUCCUCAGCCGCCGUCAGCGUCGCUCCUAC | |
| | CGCCGCCGAGAAAGUCCCCGCGGAGUGCCCCGAACUAACGCGCCGAUGCUUG | |
| | UUGGGUGAGGUGUUUGAGGGUGACAAGUAUGAAAGUUGGCUGCGCCCGUUG | |
| | GUGAAUGUUACCGGGCGCGAUGGCCCGCUAUCGCAACUUAUCCGUUACCGUC | |
| | CCGUUACGCCGGAGGCCGCCAACUCCGUGCUGUUGGACGAGGCUUUCCUGGA | |
| | CACUCUGGCCCUGCUGUACAACAAUCCGGAUCAAUUGCGGGCCCUGCUGACG | |
| | CUGUUGAGCUCGGACACAGCGCCGCGCUGGAUGACGGUGAUGCGCGGCUAC | |
| | AGCGAGUGCGGCGAUGGCUCGCCGGCCGUGUACACGUGCGUGGACGACCUG | |
| | UGCCGCGGCUACGACCUCACGCGACUGUCAUACGGGCGCAGCAUCUUCACGG | |
| | AACACGUGUUAGGCUUCGAGCUGGUGCCACCGUCUCUCUUUAACGUGGUGG | |
| | UGGCCAUACGCAACGAAGCCACGCGUACCAACCGCGCCGUGCGUCUGCCCGU | |
| | GAGCACCGCUGCCGCGCCCGAGGGCAUCACGCUCUUUUACGGCCUGUACAAC | |
| | GCAGUGAAGGAAUUCUGCCUGCGUCACCAGCUGGACCCGCCGCUGCUACGCC | |
| | ACCUAGAUAAAUACUACGCCGGACUGCCGCCCGAGCUGAAGCAGACGCGCGU | |
| | CAACCUGCCGGCUCACUCGCGCUAUGGCCCUCAAGCAGUGGAUGCUCGCUGA | |
| | UAAUAGGCUGGAGCCUCGGUGGCCAUGCUUCUUGCCCCUUGGGCCUCCCCCC | |
| | AGCCCCUCCUCCCCUUCCUGCACCCGUACCCCCGUGGUCUUUGAAUAAAGUC | |
| | UGAGUGGGCGGC | |
| hCMV-gL, | MCRRPDCGFSFSPGPVILLWCCLLLPIVSSAAVSVAPTAAEKVPAECPELTRRCLLG | 61 |
| amino acid | EVFEGDKYESWLRPLVNVTGRDGPLSQLIRYRPVTPEAANSVLLDEAFLDTLALLY | |
| sequence | NNPDQLRALLTLLSSDTAPRWMTVMRGYSECGDGSPAVYTCVDDLCRGYDLTRL | |
| | SYGRSIFTEHVLGFELVPPSLFNVVVAIRNEATRTNRAVRLPVSTAAAPEGITLFYG | |
| | LYNAVKEFCLRHQLDPPLLRHLDKYYAGLPPELKQTRVNLPAHSRYGPQAVDAR | |
| hCMV_UL | TCAAGCTTTTGGACCCTCGTACAGAAGCTAATACGACTCACTATAGGGAA | 62 |
| 128, | ATAAGAGAGAAAAGAAGAGTAAGAAGAAATATAAGAGCCACCATGAGTCC | |
| nucleotide | CAAAGATCTGACGCCGTTCTTGACGGCGTTGTGGCTGCTATTGGGTCACAGCCG | |
| sequence | CGTGCCGCGGGTGCGCGCAGAAGAATGTTGCGAATTCATAAACGTCAACCACC | |
| | CGCCGGAACGCTGTTACGATTTCAAAATGTGCAATCGCTTCACCGTCGCGCTGC | |
| | GGTGTCCGGACGGCGAAGTCTGCTACAGTCCCGAGAAAACGGCTGAGATTCGC | |
| | GGGATCGTCACCACCATGACCCATTCATTGACACGCCAGGTCGTACACAACAA | |
| | ACTGACGAGCTGCAACTACAATCCGTTATACCTCGAAGCTGACGGGCGAATAC | |
| | GCTGCGGCAAAGTAAACGACAAGGCGCAGTACCTGCTGGGCGCCGCTGGCAG | |
| | CGTTCCCTATCGATGGATCAATCTGGAATACGACAAGATAACCCGGATCGTGG | |
| | GCCTGGATCAGTACCTGGAGAGCGTTAAGAAACACAAACGGCTGGATGTGTGC | |
| | CGCGCTAAAATGGGCTATATGCTGCAGTGATAATAGGCTGGAGCCTCGGTGGC | |
| | CATGCTTCTTGCCCCTTGGGCCTCCCCCCAGCCCCTCCTCCCCTTCCTGCACCCG | |
| | TACCCCCGTGGTCTTTGAATAAAGTCTGAGTGGGCGGC | |
| hCMV_UL | UCAAGCUUUUGGACCCUCGUACAGAAGCUAAUACGACUCACUAUAGGGAAA | 191 |
| 128, | UAAGAGAGAAAAGAAGAGUAAGAAGAAAUAUAAGAGCCACCAUGAGUCCCA | |
| nucleotide | AAGAUCUGACGCCGUUCUUGACGGCGUUGUGGCUGCUAUUGGGUCACAGCC | |
| sequence | GCGUGCCGCGGGUGCGCGCAGAAGAAUGUUGCGAAUUCAUAAACGUCAACC | |
| mRNA | ACCCGCCGGAACGCUGUUACGAUUUCAAAAUGUGCAAUCGCUUCACCGUCGC | |
| | GCUGCGGUGUCCGGACGGCGAAGUCUGCUACAGUCCCGAGAAAACGGCUGA | |
| | GAUUCGCGGGAUCGUCACCACCAUGACCCAUUCAUUGACACGCCAGGUCGUA | |
| | CACAACAAACUGACGAGCUGCAACUACAAUCCGUUAUACCUCGAAGCUGACG | |
| | GGCGAAUACGCUGCGGCAAAGUAAACGACAAGGCGCAGUACCUGCUGGGCG | |
| | CCGCUGGCAGCGUUCCCUAUCGAUGGAUCAAUCUGGAAUACGACAAGAUAA | |
| | CCCGGAUCGUGGGCCUGGAUCAGUACCUGGAGAGCGUUAAGAAACACAAAC | |
| | GGCUGGAUGUGUGCCGCGCUAAAAUGGGCUAUAUGCUGCAGUGAUAAUAGG | |
| | CUGGAGCCUCGGUGGCCAUGCUUCUUGCCCCUUGGGCCUCCCCCCAGCCCCU | |
| | CCUCCCCUUCCUGCACCCGUACCCCCGUGGUCUUUGAAUAAAGUCUGAGUGG | |
| | GCGGC | |
| hCMV_UL | MSPKDLTPFLTALWLLLGHSRVPRVRAEECCEFINVNHPPERCYDFKMCNRFTVA | 63 |
| 128, amino | LRCPDGEVCYSPEKTAEIRGIVTTMTHSLTRQVVHNKLTSCNYNPLYLEADGRIRC | |
| acid | GKVNDKAQYLLGAAGSVPYRWINLEYDKITRIVGLDQYLESVKKHKRLDVCRAK | |
| sequence | MGYMLQ | |
| hCMV- | TCAAGCTTTTGGACCCTCGTACAGAAGCTAATACGACTCACTATAGGGAA | 64 |
| UL130, | ATAAGAGAGAAAAGAAGAGTAAGAAGAAATATAAGAGCCACCATGCTGCG | |
| nucleotide | GCTTCTGCTTCGTCACCACTTTCACTGCCTGCTTCTGTGCGCGGTTTGGGCAAC | |
| sequence | GCCCTGTCTGGCGTCTCCGTGGTCGACGCTAACAGCAAACCAGAATCCGTCCC | |
| | CGCCATGGTCTAAACTGACGTATTCCAAACCGCATGACGCGGCGACGTTTTACT | |
| | GTCCTTTTCTCTATCCCTCGCCCCCACGATCCCCCTTGCAATTCTCGGGGTTCCA | |
| | GCGGGTATCAACGGGTCCCGAGTGTCGCAACGAGACCCTGTATCTGCTGTACA | |
| | ACCGGGAAGGCCAGACCTTGGTGGAGAGAAGCTCCACCTGGGTGAAAAAGGT | |
| | GATCTGGTACCTGAGCGGTCGGAACCAAACCATCCTCCAACGGATGCCCCGAA | |
| | CGGCTTCGAAACCGAGCGACGGAAACGTGCAGATCAGCGTGGAAGACGCCAA | |
| | GATTTTTGGAGCGCACATGGTGCCCAAGCGCTGCTACGCTTCGTCGTCAACGAT | |
| | GGCACACGTTATCAGATGTGTGTGATGAAGCTGGAGAGCTGGGCTCACGTCTT | |
| | CCGGGACTACAGCGTGTCTTTTCAGGTGCGATTGACGTTCACCGAGGCCAATA | |
| | ACCAGACTTACACCTTCTGCACCCATCCCAATCTCATCGTTTGATAATAGGCTG | |
| | GAGCCTCGGTGGCCATGCTTCTTGCCCCTTGGGCCTCCCCCCAGCCCCTCCTCC | |
| | CCTTCCTGCACCCGTACCCCCGTGGTCTTTGAATAAAGTCTGAGTGGGCGGC | |
| hCMV- | UCAAGCUUUUGGACCCUCGUACAGAAGCUAAUACGACUCACUAUAGGGAAA | 192 |
| UL130, | UAAGAGAGAAAAGAAGAGUAAGAAGAAAUAUAAGAGCCACCAUGCUGCGGC | |
| nucleotide | UUCUGCUUCGUCACCACUUUCACUGCCUGCUUCUGUGCGCGGUUUGGGCAAC | |
| sequence | GCCCUGUCUGGCGUCUCCGUGGUCGACGCUAACAGCAAACCAGAAUCCGUCC | |
| mRNA | CCGCCAUGGUCUAAACUGACGUAUUCCAAACCGCAUGACGCGGCGACGUUUU | |
| | ACUGUCCUUUUCUCUAUCCCUCGCCCCCACGAUCCCCCUUGCAAUUCUCGGG | |
| | GUUCCAGCGGGUAUCAACGGGUCCCGAGUGUCGCAACGAGACCCUGUAUCU | |
| | GCUGUACAACCGGGAAGGCCAGACCUUGGUGGAGAGAAGCUCCACCUGGGU | |
| | GAAAAAGGUGAUCUGGUACCUGAGCGGUCGGAACCAAACCAUCCUCCAACG | |
| | GAUGCCCCGAACGGCUUCGAAACCGAGCGACGGAAACGUGCAGAUCAGCGU | |
| | GGAAGACGCCAAGAUUUUUGGAGCGCACAUGGUGCCCAAGCGCUGCUACGC | |
| | UUCGUCGUCAACGAUGGCACACGUUAUCAGAUGUGUGUGAUGAAGCUGGAG | |
| | AGCUGGGCUCACGUCUUCCGGGACUACAGCGUGUCUUUUCAGGUGCGAUUG | |
| | ACGUUCACCGAGGCCAAUAACCAGACUUACACCUUCUGCACCCAUCCCAAUC | |
| | UCAUCGUUUGAUAAUAGGCUGGAGCCUCGGUGGCCAUGCUUCUUGCCCCUU | |
| | GGGCCUCCCCCCAGCCCCUCCUCCCCUUCCUGCACCCGUACCCCCGUGGUCUU | |
| | UGAAUAAAGUCUGAGUGGGCGGC | |
| hCMV- | MLRLLLRHHFHCLLLCAVWATPCLASPWSTLTANQNPSPPWSKLTYSKPHDAATP | 65 |
| UL130, | YCPFLYPSPPRSPLQFSGFQRVSTGPECRNETLYLLYNREGQTLVERSSTWVKKVI | |
| amino acid | WYLSGRNQTILQRMPRTASKPSDGNVQISVEDAKIFGAHMVPKQTKLLRFVVNDG | |
| sequence | TRYQMCVMKLESWAHVFRDYSVSFQVRLTFTEANNQTYTFCTHPNLIV | |
| hCMV | TCAAGCTTTTGGACCCTCGTACAGAAGCTAATACGACTCACTATAGGGAA | 66 |
| UL131A, | ATAAGAGAGAAAAGAAGAGTAAGAAGAAATATAAGAGCCACCATGCGGCT | |
| nucleotide | GTGTCGGGTGTGGCTGTCTGTTTGTCTGTGCGCCGTGGTGCTGGGTCAGTGCCA | |
| sequence | GCGGGAAACCGCGGAAAAGAACGATTATTACCGAGTACCGCATTACTGGGAC | |
| | GCGTGCTCTCGCGCGCTGCCCGACCAAACCCGTTACAAGTATGTGGAACAGCT | |
| | CGTGGACCTCACGTTGAACTACCACTACGATGCGAGCCACGGCTTGGACAACT | |
| | TTGACGTGCTCAAGAGAATCAACGTGACCGAGGTGTCGTTGCTCATCAGCGAC | |
| | TTTAGACGTCAGAACCGTCGCGGCGGCACCAACAAAAGGACCACGTTCAACGC | |
| | CGCCGGTTCGCTGGCGCCACACGCCCGGAGCCTCGAGTTCAGCGTGCGGCTCT | |
| | TTGCCAACTGATAATAGGCTGGAGCCTCGGTGGCCATGCTTCTTGCCCCTTGGG | |
| | CCTCCCCCCAGCCCCTCCTCCCCTTCCTGCACCCGTACCCCCGTGGTCTTTGAAT | |
| | AAAGTCTGAGTGGGCGGC | |
| hCMV | UCAAGCUUUUGGACCCUCGUACAGAAGCUAAUACGACUCACUAUAGGGAAA | 193 |
| UL131A, | UAAGAGAGAAAAGAAGAGUAAGAAGAAAUAUAAGAGCCACCAUGCGGCUGU | |
| nucleotide | GUCGGGUGUGGCUGUCUGUUUGUCUGUGCGCCGUGGUGCUGGGUCAGUGCC | |
| sequence | AGCGGGAAACCGCGGAAAAGAACGAUUAUUACCGAGUACCGCAUUACUGGG | |
| mRNA | ACGCGUGCUCUCGCGCGCUGCCCGACCAAACCCGUUACAAGUAUGUGGAACA | |
| | GCUCGUGGACCUCACGUUGAACUACCACUACGAUGCGAGCCACGGCUUGGAC | |
| | AACUUUGACGUGCUCAAGAGAAUCAACGUGACCGAGGUGUCGUUGCUCAUC | |
| | AGCGACUUUAGACGUCAGAACCGUCGCGGCGGCACCAACAAAAGGACCACGU | |
| | UCAACGCCGCCGGUUCGCUGGCGCCACACGCCCGGAGCCUCGAGUUCAGCGU | |
| | GCGGCUCUUUGCCAACUGAUAAUAGGCUGGAGCCUCGGUGGCCAUGCUUCU | |
| | UGCCCCUUGGGCCUCCCCCCAGCCCCUCCUCCCCUUCCUGCACCCGUACCCCC | |
| | GUGGUCUUUGAAUAAAGUCUGAGUGGGCGGC | |
| hCMV | MRLCRVWLSVCLCAVVLGQCQRETAEKNDYYRVPHYWDACSRALPDQTRYKYV | 67 |
| UL131A, | EQLVDLTLNYHYDASHGLDNFDVLKRINVTEVSLLISDFRRQNRRGGTNKRTTFN | |
| amino acid | AAGSLAPHARSLEFSVRLFAN | |
| sequence | | |
| hCMV_gB, | TCAAGCTTTTGGACCCTCGTACAGAAGCTAATACGACTCACTATAGGGAA | 68 |
| nucleotide | ATAAGAGAGAAAAGAAGAGTAAGAAGAAATATAAGAGCCACCATGGAATC | |
| sequence | CAGGATCTGGTGCCTGGTAGTCTGCGTTAACTTGTGTATCGTCTGTCTGGGTGC | |
| | TGCGGTTTCCTCATCTTCTACTCGTGGAACTTCTGCTACTCACAGTCACCATTCC | |
| | TCTCATACGACGTCTGCTGCTCACTCTCGATCCGGTTCAGTCTCTCAACGCGTA | |
| | ACTTCTTCCCAAACGGTCAGCCATGGTGTTAACGAGACCATCTACAACACTACC | |
| | CTCAAGTACGGAGATGTGGTGGGGGTCAATACCACCAAGTACCCCTATCGCGT | |
| | GTGTTCTATGGCCCAGGGTACGGATCTTATTCGCTTTGAACGTAATATCGTCTG | |
| | CACCTCGATGAAGCCCATCAATGAAGACCTGGACGAGGGCATCATGGTGGTCT | |
| | ACAAACGCAACATCGTCGCGCACACCTTTAAGGTACGAGTCTACCAGAAGGTT | |
| | TTGACGTTTCGTCGTAGCTACGCTTACATCCACACCACTTATCTGCTGGGCAGC | |
| | AACACGGAATACGTGGCGCCTCCTATGTGGGAGATTCATCATATCAACAGCCA | |
| | CAGTCAGTGCTACAGTTCCTACAGCCGCGTTATAGCAGGCACGGTTTTCGTGGC | |
| | TTATCATAGGGACAGCTATGAAAACAAAACCATGCAATTAATGCCCGACGATT | |
| | ATTCCAACACCCACAGTACCCGTTACGTGACGGTCAAGGATCAATGGCACAGC | |
| | CGCGGCAGCACCTGGCTCTATCGTGAGACCTGTAATCTGAATTGTATGGTGACC | |
| | ATCACTACTGCGCGCTCCAAATATCCTTATCATTTTTTCGCCACTTCCACGGGT | |
| | GACGTGGTTGACATTTCTCCTTTCTACAACGGAACCAATCGCAATGCCAGCTAC | |
| | TTTGGAGAAAACGCCGACAAGTTTTTCATTTTTCCGAACTACACTATCGTCTCC | |
| | GACTTTGGAAGACCGAATTCTGCGTTAGAGACCCACAGGTTGGTGGCTTTTCTT | |
| | GAACGTGCGGACTCGGTGATCTCCTGGGATATACAGGACGAAAAGAATGTCAC | |
| | TTGTCAACTCACTTTCTGGGAAGCCTCGGAACGCACCATTCGTTCCGAAGCCGA | |
| | GGACTCGTATCACTTTTCTTCTGCCAAAATGACCGCCACTTTCTTATCTAAGAA | |
| | GCAAGAGGTGAACATGTCCGACTCTGCGCTGGACTGCGTACGTGATGAGGCTA | |
| | TAAATAAGTTACAGCAGATTTTCAATACTTCATACAATCAAACATATGAAAAA | |
| | TATGGAAACGTGTCCGTCTTTGAAACCACTGGTGGTTTGGTAGTGTTCTGGCAA | |
| | GGTATCAAGCAAAAATCTCTGGTGGAACTCGAACGTTTGGCCAACCGCTCCAG | |
| | TCTGAATCTTACTCATAATAGAACCAAAAGAAGTACAGATGGCAACAATGCAA | |
| | CTCATTTATCCAACATGGAATCGGTGCACAATCTGGTCTACGCCCAGCTGCAGT | |
| | TCACCTATGACACGTTGCGCGGTTACATCAACCGGGCGCTGGCGCAAATCGCA | |
| | GAAGCCTGGTGTGTGGATCAACGGCGCACCCTAGAGGTCTTCAAGGAACTCAG | |
| | CAAGATCAACCCGTCAGCCATTCTCTCGGCCATTTACAACAAACCGATTGCCGC | |
| | GCGTTTCATGGGTGATGTCTTCiGGCCTGGCCAGCTGCGTGACCATCAACCAAA | |
| | CCAGCGTCAAGGTGCTGCGTGATATGAACGTGAAGGAGTCGCCAGGACGCTGC | |
| | TACTCACGACCCGTGGTCATCTTTAATTTCGCCAACAGCTCGTACGTGCAGTAC | |
| | GGTCAACTGGGCGAGGACAACGAAATCCTGTTGGGCAACCACCGCACTGAGG | |
| | AATGTCAGCTTCCCAGCCTCAAGATCTTCATCGCCGGGAACTCGGCCTACGAGT | |
| | ACGTGGACTACCTCTTCAAACGCATGATTGACCTCAGCAGTATCTCCACCGTCG | |
| | ACAGCATGATCGCCCTGGATATCGACCCGCTGGAAAATACCGACTTCAGGGTA | |
| | CTGGAACTTTACTCGCAGAAAGAGCTGCGTTCCAGCAACGTTTTTGACCTCGAA | |
| | GAGATCATGCGCGAATTCAACTCGTACAAGCAGCGGGTAAAGTACGTGGAGG | |
| | ACAAGGTAGTCGACCCGCTACCGCCCTACCTCAAGGGTCTGGACGACCTCATG | |
| | AGCGGCCTGGGCGCCGCGGGAAAGGCCGTTGGCGTAGCCATTGGGGCCGTGG | |
| | GTGGCGCGGTGGCCTCCGTGGTCGAAGGCGTTGCCACCTTCCTCAAAAACCCC | |
| | TTCGGAGCGTTCACCATCATCCTCGTGGCCATAGCTGTAGTCATTATCACTTAT | |
| | TTGATCTATACTCGACAGCGGCGTTTGTGCACGCAGCCGCTGCAGAACCTCTTT | |
| | CCCTATCTGGTGTCCGCCGACGGGACCACCGTGACGTCGGGCAGCACCAAAGA | |
| | CACGTCGTTACAGGCTCCGCCTTCCTACGAGGAAAGTGTTTATAATTCTGGTCG | |
| | CAAAGGACCGGGACCACCGTCGTCTGATGCATCCACGGCGGCTCCGCCTTACA | |
| | CCAACGAGCAGGCTTACCAGATGCTTCTGGCCCTGGCCCGTCTGGACGCAGAG | |
| | CAGCGAGCGCAGCAGAACGGTACAGATTCTTTGGACGGACGGACTGGCACGC | |
| | AGGACAAGGGACAGAAGCCCAACCTACTAGACCGACTGCGACATCGCAAAAA | |
| | CGGCTACCGACACTTGAAAGACTCTGACGAAGAAGAGAACGTCTTGATAATAG | |
| | GCTGGAGCCTCGGTGGCCATGCTTCTTGCCCCTTGGGCCTCCCCCCAGCCCCTC | |
| | CTCCCCTTCCTGCACCCGTACCCCCGTGGTCTTTGAATAAAGTCTGAGTGGGCG | |
| | GC | |
| hCMV_gB, | UCAAGCUUUUGGACCCUCGUACAGAAGCUAAUACGACUCACUAUAGGGAAA | 194 |
| nucleotide | UAAGAGAGAAAAGAAGAGUAAGAAGAAAUAUAAGAGCCACCAUGGAAUCCA | |
| sequence | GGAUCUGGUGCCUGGUAGUCUGCGUUAACUUGUGUAUCGUCUGUCUGCGUG | |
| mRNA | CUGCGGUUUCCUCAUCUUCUACUCGUGGAACUUCUGCUACUCACAGUCACCA | |
| | UUCCUCUCAUACGACGUCUGCUGCUCACUCUCGAUCCGGUUCAGUCUCUCAA | |
| | CGCGUAACUUCUUCCCAAACGGUCAGCCAUGGUGUUAACGAGACCAUCUACA | |
| | ACACUACCCUCAAGUACGGAGAUGUGGUGGGGGUCAAUACCACCAAGUACC | |
| | CCUAUCGCGUGUGUUCUAUGGCCCAGGGUACGGAUCUUAUUCGCUUUGAAC | |
| | GUAAUAUCGUCUGCACCUCGAUGAAGCCCAUCAAUGAAGACCUGGACGAGG | |
| | GCAUCAUGGUGGUCUACAAACGCAACAUCGUCGCGCACACCUUUAAGGUAC | |
| | GAGUCUACCAGAAGGUUUUGACGUUUCGUCGUAGCUACGCUUACAUCCACA | |
| | CCACUUAUCUGCUGGGCAGCAACACGGAAUACGUGGCGCCUCCUAUGUGGG | |
| | AGAUUCAUCAUAUCAACAGCCACAGUCAGUGCUACAGUUCCUACAGCCGCGU | |
| | UAUAGCAGGCACGGUUUUCGUGGCUUAUCAUAGGGACAGCUAUGAAAACAA | |
| | AACCAUGCAAUUAAUGCCCGACGAUUAUUCCAACACCCACAGUACCCGUUAC | |
| | GUGACGGUCAAGGAUCAAUGGCACAGCCGCGGCAGCACCUGGCUCUAUCGU | |
| | GAGACCUGUAAUCUGAAUUGUAUGGUGACCAUCACUACUGCGCGCUCCAAA | |
| | UAUCCUUAUCAUUUUUUCGCCACUUCCACGGGUGACGUGGUUGACAUUUCU | |
| | CCUUUCUACAACGGAACCAAUCGCAAUGCCAGCUACUUUGGAGAAAACGCCG | |
| | ACAAGUUUUUCAUUUUUCCGAACUACACUAUCGUCUCCGACUUUGGAAGAC | |
| | CGAAUUCUGCGUUAGAGACCCACAGGUUGGUGGCUUUUCUUGAACGUGCGG | |
| | ACUCGGUGAUCUCCUGGGAUAUACAGGACGAAAAGAAUGUCACUUGUCAAC | |
| | UCACUUUCUGGGAAGCCUCGGAACGCACCAUUCGUUCCGAAGCCGAGGACUC | |
| | GUAUCACUUUUCUUCUGCCAAAAUGACCGCCACUUUCUUAUCUAAGAAGCA | |
| | AGAGGUGAACAUGUCCGACUCUGCGCUGGACUGCGUACGUGAUGAGGCUAU | |
| | AAAUAAGUUACAGCAGAUUUUCAAUACUUCAUACAAUCAAACAUAUGAAAA | |
| | AUAUGGAAACGUGUCCGUCUUUGAAACCACUGGUGGUUUGGUAGUGUUCUG | |
| | GCAAGGUAUCAAGCAAAAAUCUCUGGUGGAACUCGAACGUUUGGCCAACCG | |
| | CUCCAGUCUGAAUCUUACUCAUAAUAGAACCAAAAGAAGUACAGAUGGCAA | |
| | CAAUGCAACUCAUUUAUCCAACAUGGAAUCGGUGCACAAUCUGGUCUACGC | |
| | CCAGCUGCAGUUCACCUAUGACACGUUGCGCGGUUACAUCAACCGGGCGCUG | |
| | GCGCAAAUCGCAGAAGCCUGGUGUGUGGAUCAACGGCGCACCCUAGAGGUC | |
| | UUCAAGGAACUCAGCAAGAUCAACCCGUCAGCCAUUCUCUCGGCCAUUUACA | |
| | ACAAACCGAUUGCCGCGCGUUUCAUGGGUGAUGUCUUGGGCCUGGCCAGCU | |
| | GCGUGACCAUCAACCAAACCAGCGUCAAGGUGCUGCGUGAUAUGAACGUGA | |
| | AGGAGUCGCCAGGACGCUGCUACUCACGACCCGUGGUCAUCUUUAAUUUCGC | |
| | CAACAGCUCGUACGUGCAGUACGGUCAACUGGGCGAGGACAACGAAAUCCU | |
| | GUUGGGCAACCACCGCACUGAGGAAUGUCAGCUUCCCAGCCUCAAGAUCUUC | |
| | AUCGCCGGGAACUCGGCCUACGAGUACGUGGACUACCUCUUCAAACGCAUGA | |
| | UUGACCUCAGCAGUAUCUCCACCGUCGACAGCAUGAUCGCCCUGGAUAUCGA | |
| | CCCGCUGGAAAAUACCGACUUCAGGGUACUGGAACUUUACUCGCAGAAAGA | |
| | GCUGCGUUCCAGCAACGUUUUUGACCUCGAAGAGAUCAUGCGCGAAUUCAA | |
| | CUCGUACAAGCAGCGGGUAAAGUACGUGGAGGACAAGGUAGUCGACCCGCU | |
| | ACCGCCCUACCUCAAGGGUCUGGACGACCUCAUGAGCGGCCUGGGCGCCGCG | |
| | GGAAAGGCCGUUGGCGUAGCCAUUGGGGCCGUGGGUGGCGCGGUGGCCUCC | |
| | GUGGUCGAAGGCGUUGCCACCUUCCUCAAAAACCCCUUCGGAGCGUUCACCA | |
| | UCAUCCUCGUGGCCAUAGCUGUAGUCAUUAUCACUUAUUUGAUCUAUACUC | |
| | GACAGCGGCGUUUGUGCACGCAGCCGCUGCAGAACCUCUUUCCCUAUCUGGU | |
| | GUCCGCCGACGGGACCACCGUGACGUCGGGCAGCACCAAAGACACGUCGUUA | |
| | CAGGCUCCGCCUUCCUACGAGGAAAGUGUUUAUAAUUCUGGUCGCAAAGGA | |
| | CCGGGACCACCGUCGUCUGAUGCAUCCACGGCGGCUCCGCCUUACACCAACG | |
| | AGCAGGCUUACCAGAUGCUUCUGGCCCUGGCCCGUCUGGACGCAGAGCAGCG | |
| | AGCGCAGCAGAACGGUACAGAUUCUUUGGACGGACGGACUGGCACGCAGGA | |
| | CAAGGGACAGAAGCCCAACCUACUAGACCGACUGCGACAUCGCAAAAACGGC | |
| | UACCGACACUUGAAAGACUCUGACGAAGAAGAGAACGUCUUGAUAAUAGGC | |
| | UGGAGCCUCGGUGGCCAUGCUUCUUGCCCCUUGGGCCUCCCCCCAGCCCCUC | |
| | CUCCCCUUCCUGCACCCGUACCCCCGUGGUCUUUGAAUAAAGUCUGAGUGGG | |
| | CGGC | |
| hCMV_gB, | MESRIWCLVVCVNLCIVCLGAAVSSSSTRGTSATHSHHSSHTTSAAHSRSGSVSQR | 69 |
| amino acid | VTSSQTVSHGVNETIYNTTLKYGDVVGVNTTKYPYRVCSMAQGTDLIRFERNIVC | |
| sequence | TSMKPINEDLDEGIMVVYKRNIVAHTFKVRVYQKVLTFRRSYAYIHTTYLLGSNTE | |
| | YVAPPMWEIHHINSHSQCYSSYSRVIAGTVFVAYHRDSYENKTMQLMPDDYSNTH | |
| | STRYVTVKDQWHSRGSTWLYRETCNLNCMVTITTARSKYPYHFFATSTGDVVDIS | |
| | PFYNGTNRNASYFGENADKFFIFPNYTIVSDFGRPNSALETHRLVAFLERADSVISW | |
| | DIQDEKNVTCQLTFWEASERTIRSEAEDSYHFSSAKMTATFLSKKQEVNMSDSALD | |
| | CVRDEAINKLQQIFNTSYNQTYEKYGNVSVFETTGGLVVFWQGIKQKSLVELERL | |
| | ANRSSLNLTHNRTKRSTDGNNATHLSNMESVHNLVYAQLQFTYDTLRGYINRALA | |
| | QIAEAWCVDQRRTLEVFKELSKINPSAILSAIYNKPIAARFMGDVLGLASCVTINQT | |
| | SVKVLRDMNVKESPGRCYSRPVVIFNFANSSYVQYGQLGEDNEILLGNHRTEECQ | |
| | LPSLKIFIAGNSAYEYVDYLFKRMIDLSSISTVDSMIALDIDPLENTDFRVLELYSQK | |
| | ELRSSNVFDLEEIMREFNSYKQRVKYVEDKVVDPLPPYLKGLDDLMSGLGAAGKA | |
| | VGVAIGAVGGAVASVVEGVATFLKNPFGAFTIILVAIAVVIITYLIYTRQRRLCTQP | |
| | LQNLFPYLVSADGTTVTSGSTKDTSLQAPPSYEESVYNSGRKGPGPPSSDASTAAPP | |
| | YTNEQAYQMLLALARLDAEQRAQQNGTDSLDGRTGTQDKGQKPNLLDRLRHRK | |
| | NGYRHLKDSDEEENV | |

### Example 26: 2A Peptide Linked Pentameric Subunits

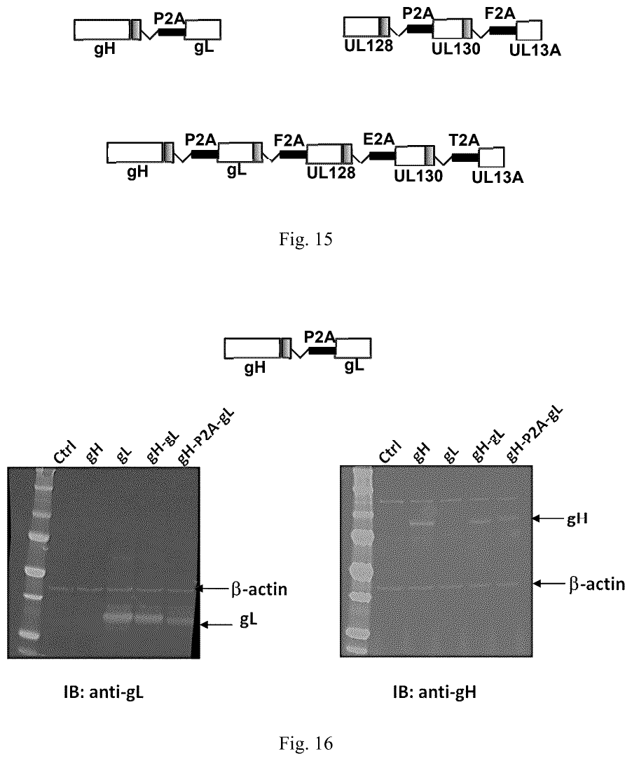

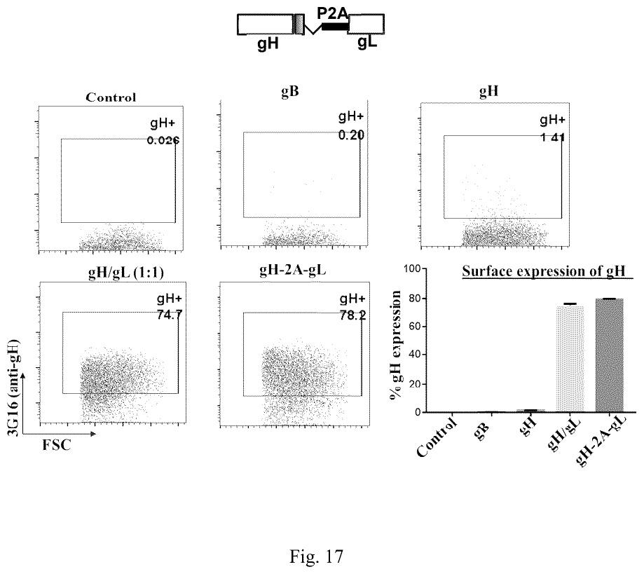

Multivalent mRNA vaccine constructs encoding the subunits of the hCMV pentamer (gH, gL, UL128, UL130, and U1131A) were designed. The multivalent mRNA encoded pentamer subunits were linked with 2A self-cleaving peptides (FIG. 15), which allows the linked subunits to process into individual subunits. 1 μg of the mRNA vaccine constructs encoding a 2A peptide linked gH-gL were transfected into 293T cells. The cells were harvested 24 hours post transfection and the cleavage of the 2A peptide were analyzed by detecting individual gH or gL subunits using Western blotting. Individual gH and gL were detected, indicating successful expression of the construct and cleavage of the 2A peptide (FIG. 16). Further, processed gH or gL when expressed in HeLa cells, dimerized, and translocated to the cell surface 24 hours after the Hela cells were transfected with 0.5 μg of mRNA encoding the 2A linked gH-gL (FIG. 17).

### Example 27: Comparison of Equimolar Vs Equal Mass of Pentamer

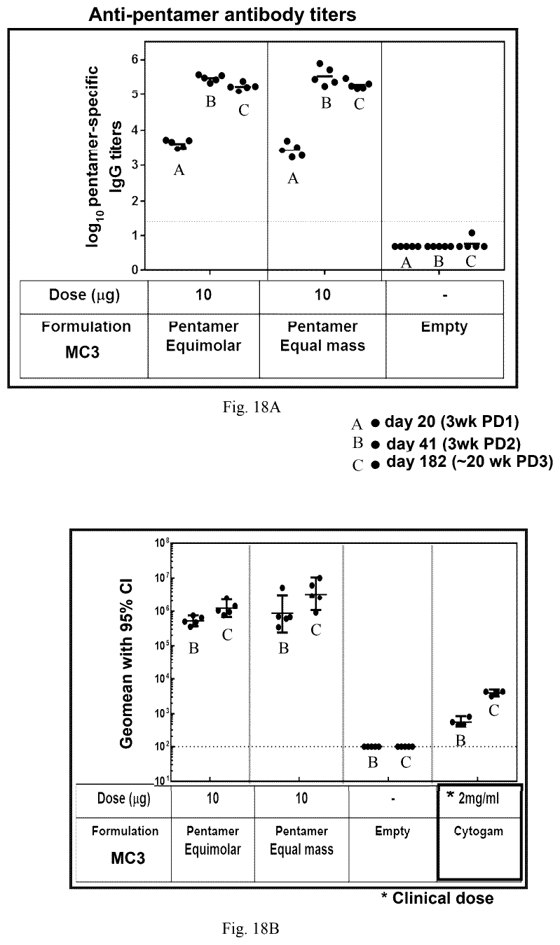

Pentameric formulations containing the pentameric subunit mRNAs at equimolar concentrations were compared to pentameric formulations containing the pentameric subunit mRNAs in equal mass. FIG. 18 demonstrates high and sustained titers of anti-pentamer binding and neutralizing antibodies in mice. FIG. 18A depicts a graph showing anti-pentamer antibody titers. Equimolar and equal mass formulations of the pentameric mRNAs were found to be equally effective. FIG. 18B depicts a graph showing neutralizing titers measured on ARPE19 epithelial cells infected with hCMV strain VR1814. Equimolar and equal mass formulations of the pentameric mRNAs were compared and were found to be equally effective. Neutralizing titers were found to be approximately 25 fold higher than CytoGam®.

### Example 28: Neutralization Activity is Dependent on Anti-Pentamer Antibodies

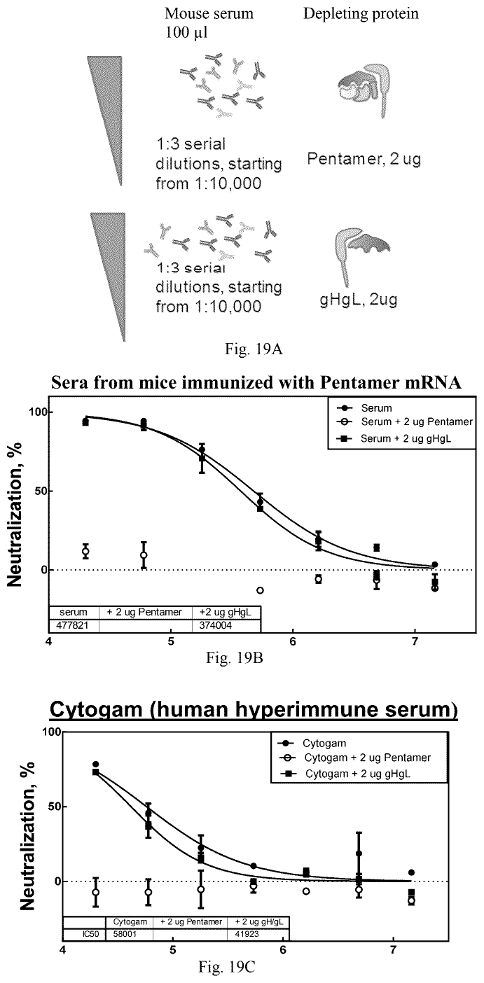

Neutralization data was assessed and compared against CytoGam®. FIG. 19 demonstrates that neutralization activity against epithelial cell infection is dependent on anti-pentamer antibodies. FIG. 19A shows that the depleting protein was either the pentamer or a gH/gL dimer. FIG. 19B and FIG. 19C depict graphs showing neutralization. FIG. 19B shows neutralization by sera from mice immunized with the pentamer or with a gH/gL dimer. FIG. 19C shows neutralization by CytoGam® combined with the pentamer or with a gH/gL.

### Example 29:Phase 1 Clinical Trial

A phase 1 clinical trial is conducted to assess the safety of the hCMV mRNA vaccine encoding the pentameric complex (gH, gL, UL128, UL130, and UL131A)+gB in humans and to evaluate the ability of the hCMV mRNA vaccines to induce an immune response. One hundred and twenty (120) volunteers (both females and males) between ages 18-49 are enrolled in the clinical trial. The volunteers are tested for CMV prior to the start of the clinical trial. Sixty (60) of the healthy volunteers are CMV⁺, while the other sixty (60) are CMV⁻.

The healthy volunteers are divided into three dosage groups, each dosage group receiving a different dose of the hCMV mRNA vaccine (e.g., low, medium, or high). For each dosage group (n=40), the hCMV mRNA vaccine is administered intramuscularly (IM, n=20) or intravenously (IV, n=20). Thus, the 120 volunteers are placed into 6 groups (referred to as a “dose arm”): low dose-IM (n=20), low dose-IV (n=20), medium dose-IM (n=20), medium dose-IV (n=20), high dose-IM (n=20), high dose-IV (n=20). In each dose arm, the volunteers are separated into two cohorts: the safety cohort (n=4, 2 receiving vaccines and 2 receiving placebos); and the expansion cohort (n=16, 13 receiving vaccines and 3 receiving placebos). The immunization of the volunteers in the expansion cohort starts 7 days after the last healthy volunteer in the safety cohort has been immunized. hCMV vaccines or placebos are given to the volunteers in the 6 dose arms on day 1, day 31, and day 61. It is a double blind clinical trial. The volunteers are followed up to a year. Blood samples are taken on day 1, day 8, day 22, day 30, day 44, 6 months, and 1 year after the first immunization.

Neutralizing hCMV antibody titers in the blood samples are measured using an Enzyme-linked ImmunoSpot (ELISPOT) assay or using a low cytometric intracellular cytokine staining (ICS) assay. Sustained neutralization antibody titers and strong anamnestic responses are expected in volunteers who received the hCMV mRNA vaccines by 12 months. The level of IgG induced by the hCMV mRNA vaccines are expected to be at least 4 times above the baseline (a clinical endpoint). The neutralization antibody titer in the blood samples of volunteers who received the hCMV mRNA vaccine, measured in a plaque reduction neutralization test (PRNT50) in both epithelial and fibroblast cells, is expected to be higher than that of CytoGam® (a clinical end point). Early signal of efficacy (ESOE) can also be indicated by measuring the viral load in urine and saliva of the volunteers by PCR on day 1, 6 months, and 12 months.

Parameters indicating safety of the vaccine are measured. Immunized volunteers are evaluated for clinical signs of hCMV infection (a clinical endpoint). Biochemical assays are performed to assess the coagulation parameters and the blood level of C-reactive proteins (CRP). The hCMV mRNA vaccine is expected to be safe.

Once safety and immunogenicity have been demonstrated, trials are conducted among target populations in phase 2 clinical trials. In some embodiments, suitable dose levels chosen from phase 1 trials will be used in phase 2 trials.

### Example 30: Phase 2 Clinical Trial

The Phase 2 trial is designed to evaluate the hCMV mRNA vaccines in the target population, e.g., seronegative transplant patients that have received solid organ transplants (SOT, e.g., kidney transplant) from a seropositive donor, and/or seropositive patients who have received a hematopoietic stem cell transplant (HCT) from a seronegative donor; and/or seropositive transplant patients that have received solid organ transplants (SOT, e.g., kidney transplant) from a seropositive donor.

Four hundred (400) patients are enrolled in the Phase 2 clinical trial and are grouped as described in the phase 1 clinical trial described in Example 28. All patients are immunized with the same dosage of hCMV mRNA vaccine. Patients receive the first dose of the vaccine on day 1, which is 2-4 weeks prior to the initiation of immunosuppressive therapy, and receive boosts at 1, 3, and 6 months post transplant. It is a double blind clinical trial. The patients are followed up to a year. Blood samples are taken on day 1, day 8, day 22, day 30, day 44, 6 months, and 1 year after the first immunization.

The safety and immunogenicity of the vaccines are assessed using methods described in the phase 1 trial, described in Example 28. A vaccine efficacy of at least 70% is expected. One endpoint of the phase 2 trial is incidence of CMV viremia by central PCR assay. If the plasma viral load is over 1000 IU/ml within 12 months of trial initiation, the patient may be determined to have viremia and may be withdrawn from the trial. Other endpoints include: (i) incidence of CMV viremia by central PCR assay defined as plasma viral load≥LLOQ (lower limit of quantification); (ii) incidence of CMV disease; (iii)incidence of adjudicated antiviral therapy of the treatment of CMV graft survival, and (iv) generation of pp65-specific DC4/CS8 responses.

The hCMV mRNA vaccine is expected to induce immune response and generate neutralizing antibodies. The safety profile is also expected to be high.

### Example 31: Phase 3 Clinical Trial

The target population of the Phase 3 trial is patients receiving solid organ transplants (SOT) or hematopoietic stem cell transplant (HCT). No CMV screening is performed prior to enrollment.

### Example 32: Evaluation of Humoral and Cellular Immunity Following Intramuscular Vaccination

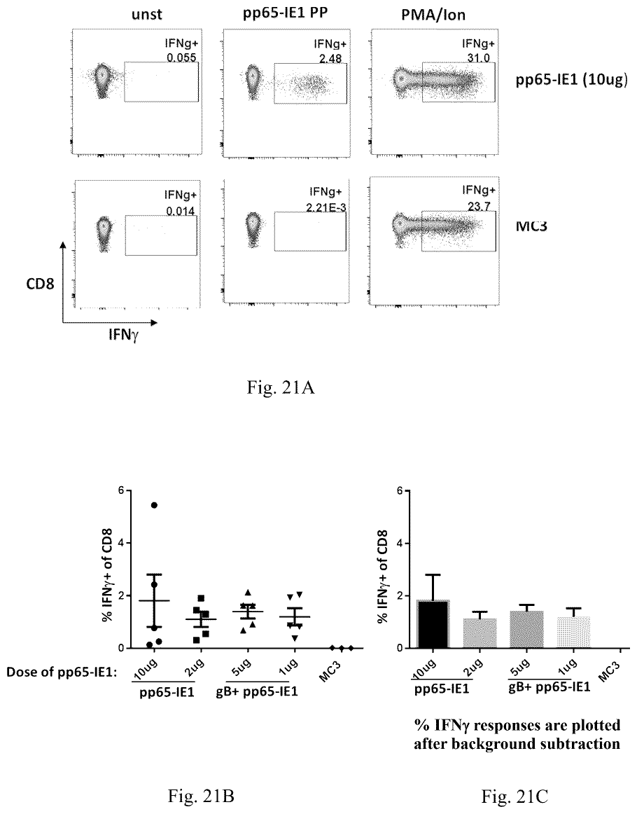

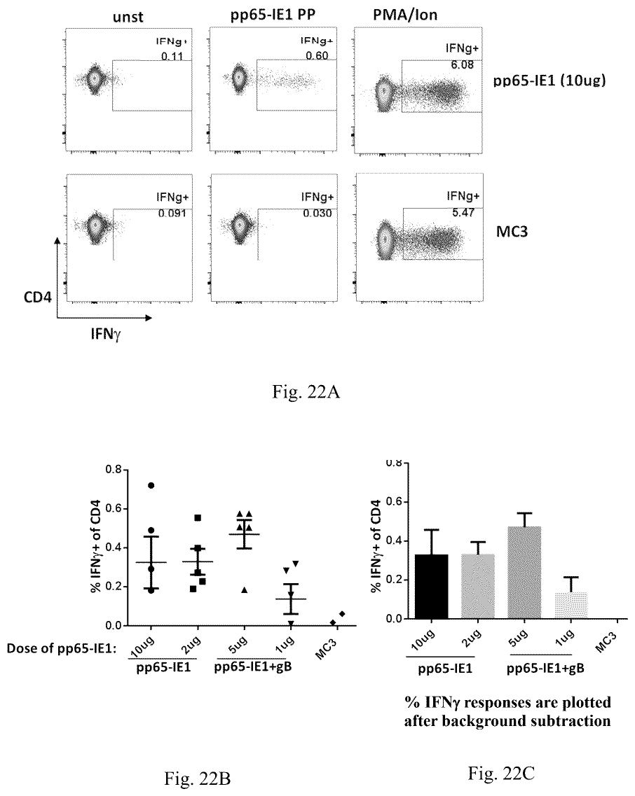

Balb/c mice were immunized with pp65-IE1, or co-immunized with gB/pp65-IE1 mRNA vaccine constructs at the indicated time points with the indicated dosages by intramuscular administration, as described in Table 7. Splenocytes were isolated for T-cell (CD4 and CD8) IFNγ response analyses. Mice splenocytes were stimulated with pp65-IE1 peptide pools and the induction of INFγ was measured by FACS on a flow cytometer (FIGS. 21A and 22A). PMA+ionomycin was used to stimulate nonspecific T-cell IFNγ response (FIGS. 20A and 21A, right panels). The results showed that splenocytes from mice immunized with pp65-IE1 mRNA vaccines produced IFNγ upon pp65-IE1 peptide pool stimulation, while splenocytes from mice immunized with empty MC3 lipid nanoparticles did not produce IFNγ upon pp65-IE peptide pool stimulation indicating that immunization with pp65-IE1 mRNAs afforded immunity against hCMV. The induction of IFNγ in splenocytes isolated from mice immunized with different formulations of mRNA vaccine constructs were plotted using the Kapil Bahl-ICS protocol (FIGS. 21B and 22B) or as a bar graph after background subtraction (FIGS. 21C and 22C).

TABLE 7
Evaluation of humoral and cellular immunity following intramuscular
vaccination

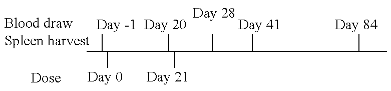

| Group | Vaccine | Route | Dose schedule | N |
|---|---|---|---|---|
| 1 | gB | IM | d 0 | 5 |
| | | | 10 ug | |
| 2 | gB | IM | d 0, d 21 | 5 |
| | | | 10 ug, 3 ug, 1 ug | |
| 5 | gB | IM | d 0, d 21 | 5 |
| | protein/MF59 | | 10 ug | |
| 6 | pp65-IE1 | IM | d 0, d 21 | 5 |
| | | | 10 ug, 2 ug | |
| 8 | gB + pp65 + IE1 | IM | d 0, d 21 | 5 |
| | (1:1), Equal conc | | 10 ug, 2 ug | |
| 11 | Empty LNP | IM | d 0, d 21, d 42 | 5 |

### Example 33: Combining hCMV Pentameric Complex mRNA Vaccine with mRNA Constructs Encoding Other Antigens

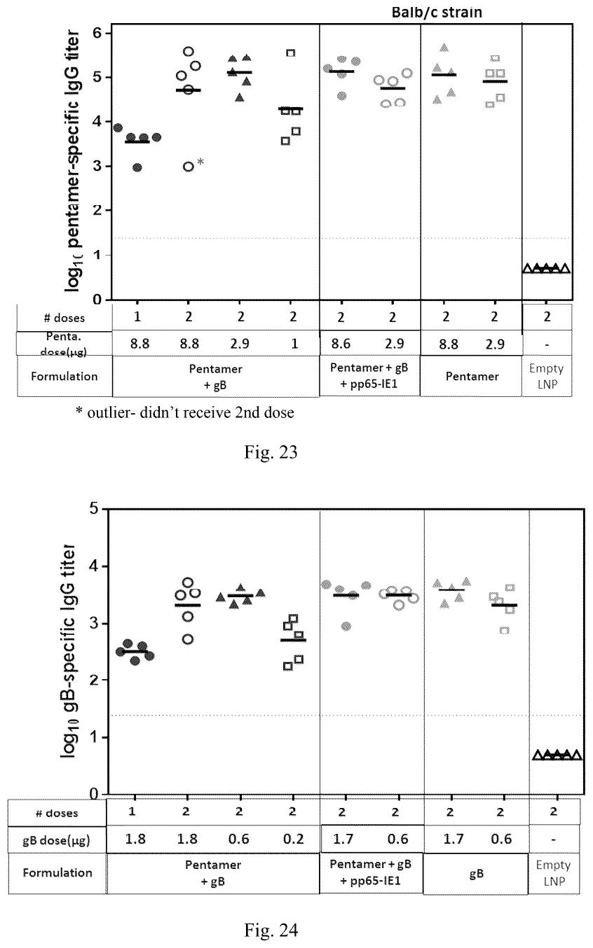

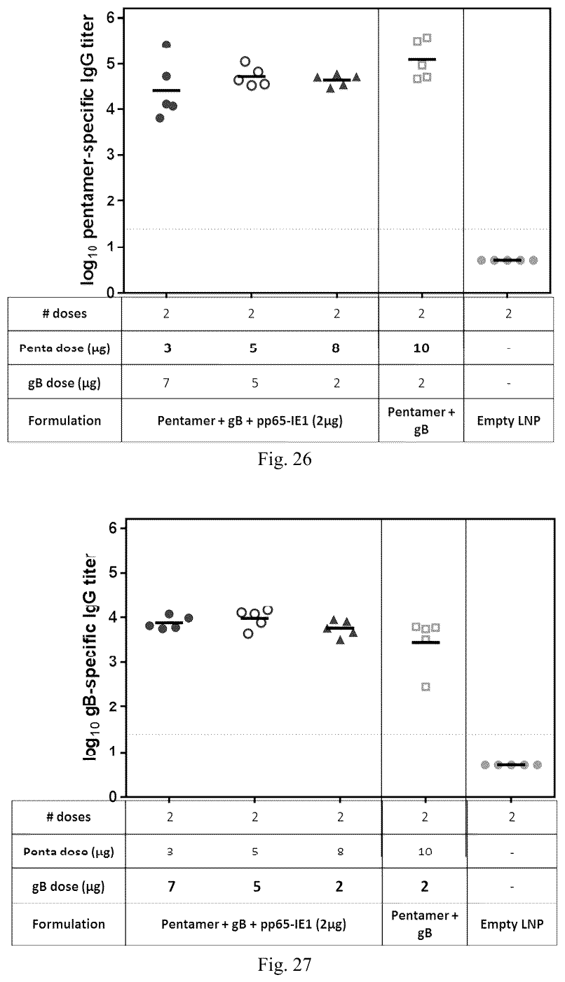

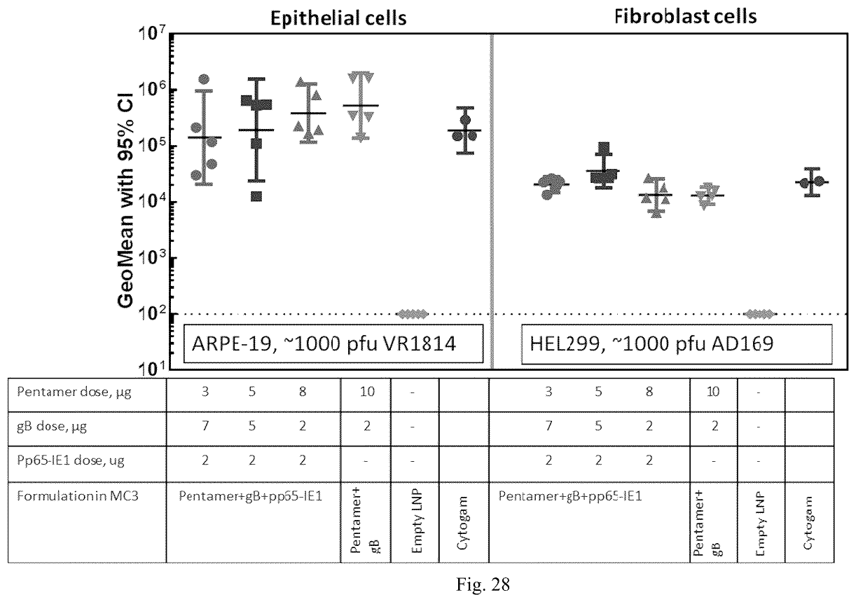

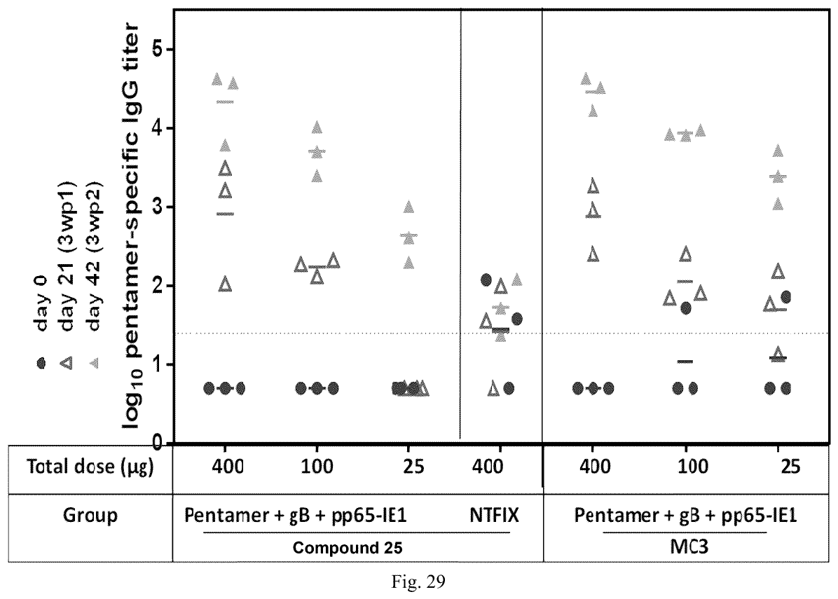

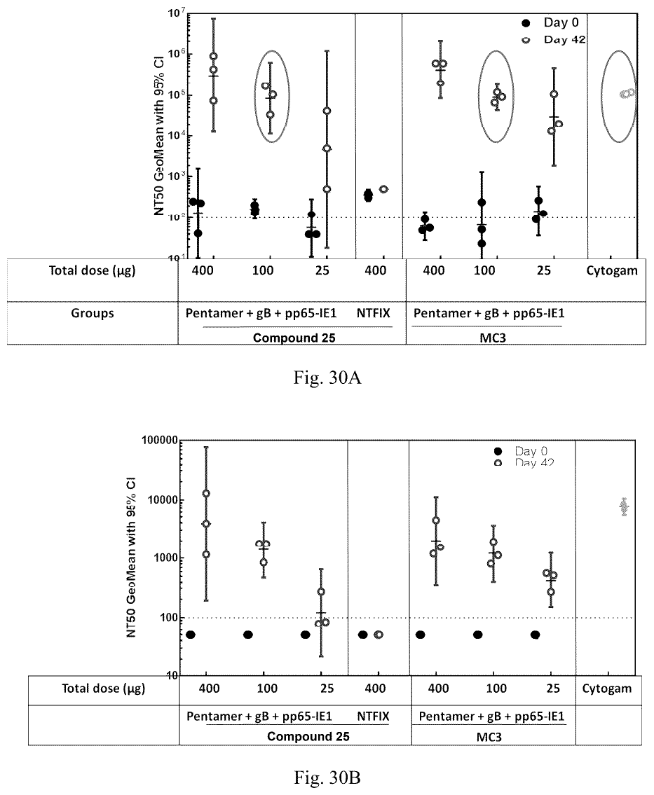

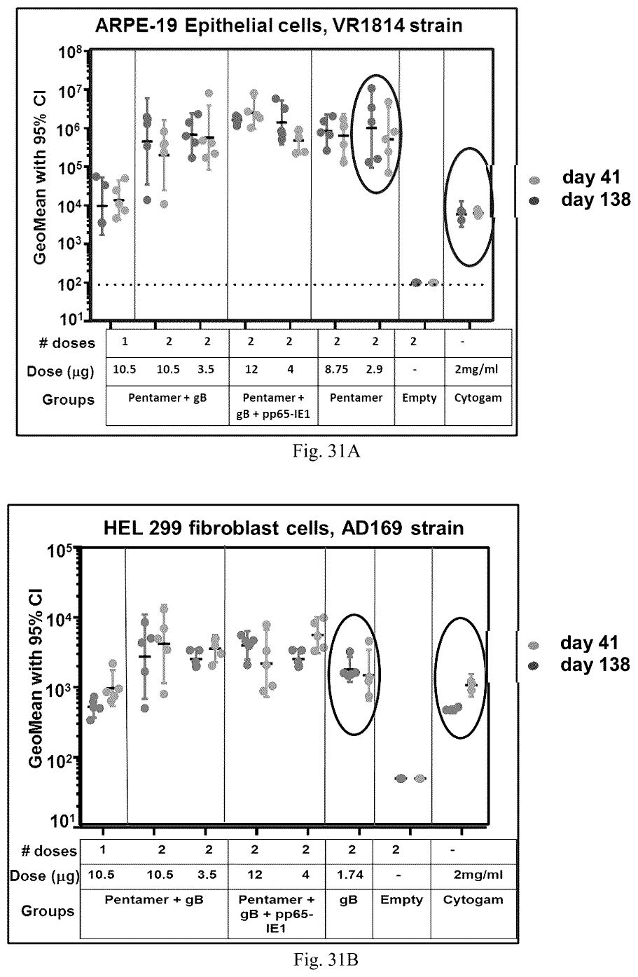

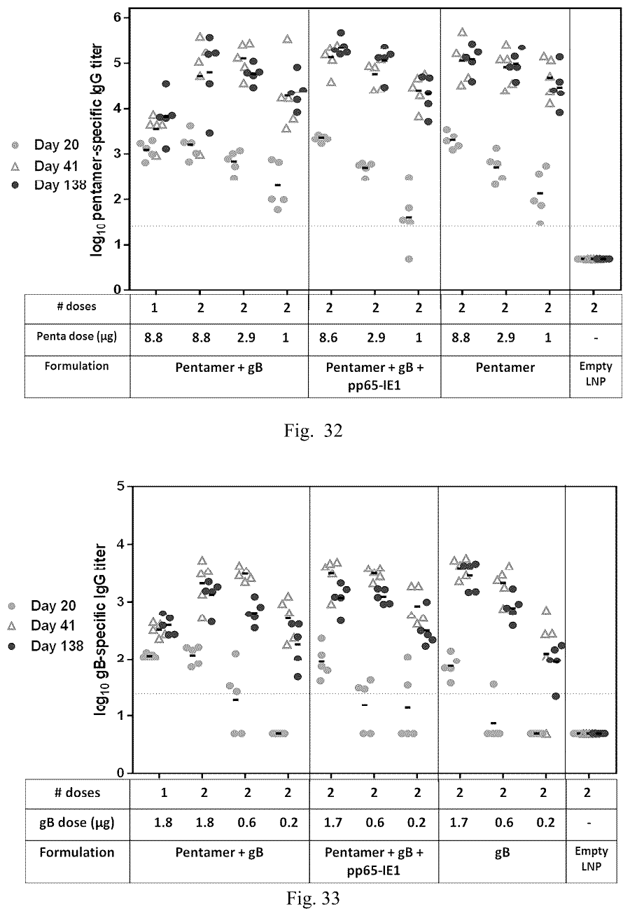

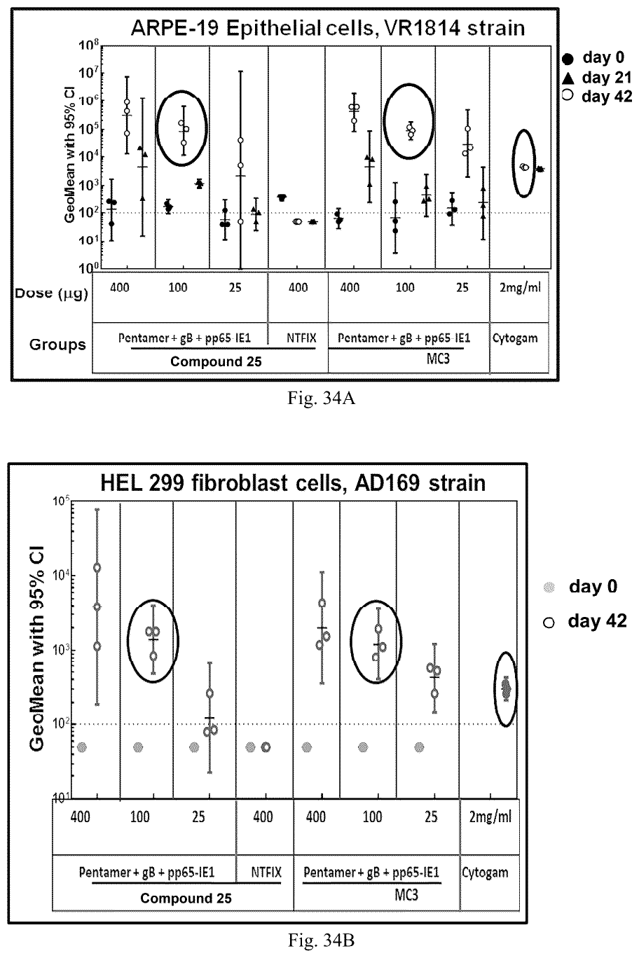

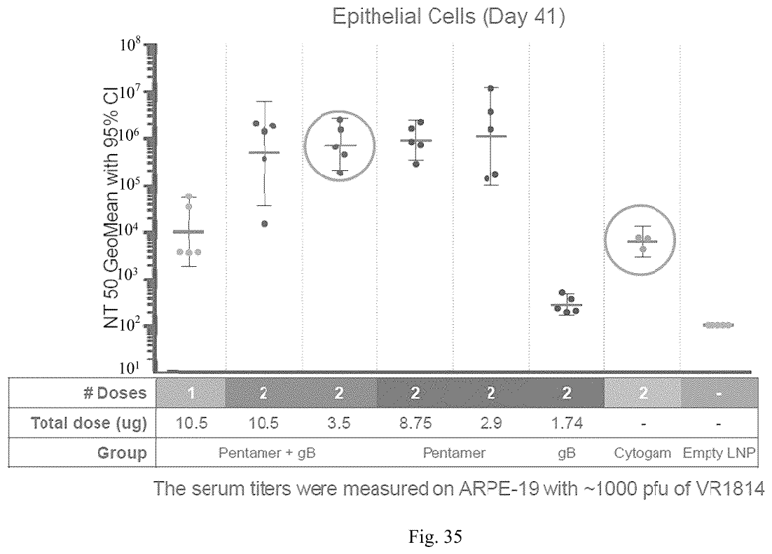

The hCMV pentameric mRNA constructs were combined with mRNA constructs encoding gB and other hCMV antigens (e.g., pp65, and/or pp65-IE1, sequences shown in Tables 8 and 9) for immunization of Balb/c mice. Mice serum were taken at day 41 post immunization and assayed on pentamer coated plates for the assessment of pentamer-specific IgG titer. Addition of mRNA constructs encoding other antigens did not affect the induction of pentamer-specific IgG (FIG. 23 and FIG. 26). Similarly, when mice serum was assayed on gB-coated plates, the results showed that the addition of mRNAs encoding the pentamer and pp65-IE1 did not affect the induction of gB-specific IgG (FIG. 24 and FIG. 27). The level of neutralizing antibodies induced by mRNA vaccine constructs encoding hCMV pentamer, gB, and pp65-IE1 was also assessed, as shown in FIGS. 15-16. The combination of mRNA constructs encoding the pentamer, gB, and pp65-IE1 induced high neutralizing antibody titers against hCMV infection (FIG. 28). Different ratios of gB:pentamer were also tested (Table 10).

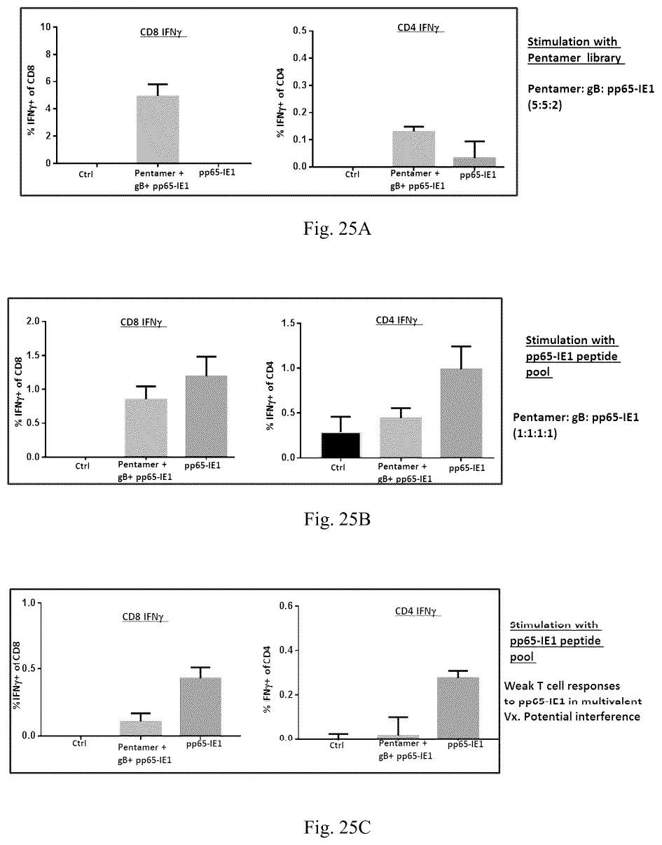

Next, antigen-specific T cell responses were assessed in the splenic lymphocytes of Balb/c mice immunized with hCMV mRNA vaccines encoding hCMV pentamer, gB, and pp65-IE1. The splenic lymphocytes were stimulated with a pentamer peptide library or a pp65-IE1 peptide pool. When the mRNA vaccine used to immunize the mice was pentamer (5 μg):gB (5 μg): pp65-IE1 (2 μg), robust CD8 response was stimulated by the pentamer peptide library (FIG. 25A). When the mRNA vaccine used to immunize the mice was pentamer (1 μg):gB (1 μg): pp65-IE1 (1 μg), CD8 (left panel) and CD4 (right panel) T cell responses were induced by a pp65-IE1 peptide pool (FIG. 25B). In summary, immunization with mRNA vaccine constructs encoding the hCMV pentamer elicited robust T cell responses. Equal concentrations of all mRNAs within the vaccine are likely to be effective as a prophylactic vaccine.

Each of the sequences described herein encompasses a chemically modified sequence or an unmodified sequence which includes no nucleotide modifications.

TABLE 8
Second Generation hCMV pp65 mRNA Vaccine Constructs Sequences

| Name of re- | | |
|---|---|---|
| designed | | |
| mRNA | | SEQ |
| Construct | Sequence | ID NO |
| pp65 phos | TCAAGCTTTTGGACCCTCGTACAGAAGCTAATACGACTCACTATAGGGAAATA | 70 |
| mut_DX, | AGAGAGAAAAGAAGAGTAAGAAGAAATATAAGAGCCACCATGGAGTCGCGCG | |
| nucleotide | GTCGCCGTTGTCCCGAAATGATATCCGTACTGGGTCCCATTTCGGGGCACGTGC | |
| sequence | TGAAAGCCGTGTTTAGTCGCGGCGATACGCCGGTGCTGCCGCACGAGACGAGA | |
| | CTCCTGCAGACGGGTATCCACGTACGCGTGAGCCAGCCCTCGCTGATCCTGGT | |
| | GTCGCAGTACACGCCCGACTCGACGCCATGCCACCGCGGCGACAATCAGCTGC | |
| | AGGTGCAGCACACGTACTTTACGGGCAGCGAGGTGGAGAACGTGTCGGTCAAC | |
| | GTGCACAACCCCACGGGCCGAAGCATCTGCCCCAGCCAAGAGCCCATGTCGAT | |
| | CTATGTGTACGCGCTGCCGCTCAAGATGCTGAACATCCCCAGCATCAACGTGC | |
| | ACCACTACCCGTCGGCGGCCGAGCGCAAACACCGACACCTGCCCGTAGCCGAC | |
| | GCTGTTATTCACGCGTCGGGCAAGCAGATGTGGCAGGCGCGTCTCACGGTCTC | |
| | GGGACTGGCCTGGACGCGTCAGCAGAACCAGTGGAAAGAGCCCGACGTCTACT | |
| | ACACGTCAGCGTTCGTGTTTCCCACCAAGGACGTGGCACTGCGGCACGTGGTG | |
| | TGCGCGCACGAGCTGGTTTGCTCCATGGAGAACACGCGCGCAACCAAGATGCA | |
| | GGTGATAGGTGACCAGTACGTCAAGGTGTACCTGGAGTCCTTCTGCGAGGACG | |
| | TGCCCTCCGGCAAGCTCTTTATGCACGTCACGCTGGGCTCTGACGTGGAAGAG | |
| | GACCTAACGATGACCCGCAACCCGCAACCCTTCATGCGCCCCCACGAGCGCAA | |
| | CGGCTTTACGGTGTTGTGTCCCAAAAATATGATAATCAAACCGGGCAAGATCT | |
| | CGCACATCATGCTGGATGTGGCTTTTACCTCACACGAGCATTTTGGGCTGCTGT | |
| | GTCCCAAGAGCATCCCGGGCCTGAGCATCTCAGGTAACCTGTTGATGAACGGG | |
| | CAGCAAATCTTCCTGGAGGTACAAGCGATACGCGAGACCGTGGAACTGCGTCA | |
| | GTACGATCCCGTGGCTGCGCTCTTCTTTTTCGATATCGACTTGTTGCTGCAGCG | |
| | CGGGCCTCAGTACAGCGAGCACCCCACCTTCACCAGCCAGTATCGCATCCAGG | |
| | GCAAGCTTGAGTACCGACACACCTGGGACCGGCACGACGAGGGTGCCGCCCA | |
| | GGGCGACGACGACGTCTGGACCAGCGGATCGGACTCCGACGAAGAACTCGTA | |
| | ACCACCGAGCGTAAGACGCCCCGCGTCACCGGCGGCGGAGCCATGGCGAGCG | |
| | CCTCCACTTCCGCGGGCTCAGCATCCTCGGCGACGGCGTGCACGGCGGGCGTT | |
| | ATGACACGCGGCCGCCTTAAGGCCGAGTCCACCGTCGCGCCCGAAGAGGACAC | |
| | CGACGAGGATTCCGACAACGAAATCCACAATCCGGCCGTGTTCACCTGGCCGC | |
| | CCTGGCAGGCCGGCATCCTGGCCCGCAACCTGGTGCCCATGGTGGCTACGGTT | |
| | CAGGGTCAGAATCTGAAGTACCAGGAGTTCTTCTGGGACGCCAACGACATCTA | |
| | CCGCATCTTCGCCGAATTGGAAGGCGTATGGCAGCCCGCTGCGCAACCCAAAC | |
| | GTCGCCGCCACCGGCAAGACGCCTTGCCCGGGCCATGCATCGCCTCGACGCCC | |
| | AAAAAGCACCGAGGTTGATAATAGGCTGGAGCCTCGGTGGCCATGCTTCTTGC | |
| | CCCTTGGGCCTCCCCCCAGCCCCTCCTCCCCTTCCTGCACCCGTACCCCCGTGG | |
| | TCTTTGAATAAAGTCTGAGTGGGCGGC | |
| pp65 phos | MESRGRRCPEMISVLGPISGHVLKAVFSRGDTPVLPHETRLLQTGIHVRVSQPSLIL | 71 |
| mut_DX, | VSQYTPDSTPCHRGDNQLQVQHTYFTGSEVENVSVNVHNPTGRSICPSQEPMSIYV | |
| amino acid | YALPLKMLNIPSINVHHYPSAAERKHRHLPVADAVIHASGKQMWQARLTVSGLA | |
| sequence | WTRQQNQWKEPDVYYTSAFVFPTKDVALRHVVCAHELVCSMENTRATKMQVIG | |
| | DQYVKVYLESFCEDVPSGKLFMHVTLGSDVEEDLTMTRNPQPFMRPHERNGFTVL | |
| | CPKNMIIKPGKISHIMLDVAFTSHEHFGLLCPKSIPGLSISGNLLMNGQQIFLEVQAI | |
| | RETVELRQYDPVAALFFFDIDLLLQRGPQYSEHPTFTSQYRIQGKLEYRHTWDRHD | |
| | EGAAQGDDDVWTSGSDSDEELVTTERKTPRVTGGGAMASASTSAGSASSATACT | |
| | AGVMTRGRLKAESTVAPEEDTDEDSDNEIHNPAVFTWPPWQAGILARNLVPMVA | |
| | TVQGQNLKYQEFFWDANDIYRIFAELEGVWQPAAQPKRRRHRQDALPGPCIASTP | |
| | KKHRG | |

TABLE 9
hCMV pp-IE1 Fusion Sequences

| Construct | Sequence, NT (5′ UTR, | ORF Sequence, | | mRNA Sequence |
|---|---|---|---|---|
| Name | ORF, 3′ UTR) | AA | ORF Sequence, NT | (assumes T100 tail) |
| hCMV_pp | TCAAGCTTTTGGACC | MESRGRRCPE | ATGGAGTCGCGCGGT | G*GGGAAATAAGAGA |
| 65-IE1 | CTCGTACAGAAGCT | MISVLGPISGH | CGCCGTTGTCCCGAA | GAAAAGAAGAGTAA |
| | AATACGACTCACTAT | VLKAVFSRGD | ATGATATCCGTACTG | GAAGAAATATAAGA |
| | AGGGAAATAAGAGA | TPVLPHETRL | GGTCCCATTTCGGGG | GCCACCATGGAGTCG |
| | GAAAAGAAGAGTAA | LQTGIHVRVS | CACGTGCTGAAAGCC | CGCGGTCGCCGTTGT |
| | GAAGAAATATAAGA | QPSLILVSQYT | GTGTTTAGTCGCGGC | CCCGAAATGATATCC |
| | GCCACCATGGAGTC | PDSTPCHRGD | GATACGCCGGTGCTG | GTACTGGGTCCCATT |
| | GCGCGGTCGCCGTT | NQLQVQHTYF | CCGCACGAGACGCGA | TCGGGGCACGTGCTG |
| | GTCCCGAAATGATA | TGSEVENVSV | CTCCTGCAGACGGGT | AAAGCCGTGTTTAGT |
| | TCCGTACTGGGTCCC | NVHNPTGRSI | ATCCACGTACGCGTG | CGCGGCGATACGCCG |
| | ATTTCGGGGCACGT | CPSQEPMSIY | AGCCAGCCCTCGCTG | GTGCTGCCGCACGAG |
| | GCTGAAAGCCGTGT | VYALPLKML | ATCCTGGTGTCGCAG | ACGCGACTCCTGCAG |
| | TTAGTCGCGGCGAT | NIPSINVHHYP | TACACGCCCGACTCG | ACGGGTATCCACGTA |
| | ACGCCGGTGCTGCC | SAAERKHRHL | ACGCCATGCCACCGC | CGCGTGAGCCAGCCC |
| | GCACGAGACGCGAC | PVADAVIHAS | GGCGACAATCAGCTG | TCGCTGATCCTGGTG |
| | TCCTGCAGACGGGT | GKQMWQARL | CAGGTGCAGCACACG | TCGCAGTACACGCCC |
| | ATCCACGTACGCGT | TVSGLAWTR | TACTTTACGGGCAGC | GACTCGACGCCATGC |
| | GAGCCAGCCCTCGC | QQNQWKEPD | GAGGTGGAGAACGT | CACCGCGGCGACAAT |
| | TGATCCTGGTGTCGC | VYYTSAFVFP | GTCGGTCAACGTGCA | CAGCTGCAGGTGCAG |
| | AGTACACGCCCGAC | TKDVALRHV | CAACCCCACGGGCCG | CACACGTACTTTACG |
| | TCGACGCCATGCCA | VCAHELVCS | AAGCATCTGCCCCAG | GGCAGCGAGGTGGA |
| | CCGCGGCGACAATC | MENTRATKM | CCAAGAGCCCATGTC | GAACGTGTCGGTCAA |
| | AGCTGCAGGTGCAG | QVIGDQYVKV | GATCTATGTGTACGC | CGTGCACAACCCCAC |
| | CACACGTACTTTACG | YLESPCEDVP | GCTGCCGCTCAAGAT | GGGCCGAAGCATCTG |
| | GGCAGCGAGGTGGA | SGKLFMHVTL | GCTGAACATCCCCAG | CCCCAGCCAAGAGCC |
| | GAACGTGTCGGTCA | GSDVEEDLTM | CATCAACGTGCACCA | CATGTCGATCTATGT |
| | ACGTGCACAACCCC | TRNPQPFMRP | CTACCCGTCGGCGGC | GTACGCGCTGCCGCT |
| | ACGGGCCGAAGCAT | HERNGFTVLC | CGAGCGCAAACACCG | CAAGATGCTGAACAT |
| | CTGCCCCAGCCAAG | PKNMIIKPGKI | ACACCTGCCCGTAGC | CCCCAGCATCAACGT |
| | AGCCCATGTCGATCT | SHIMLDVAFT | CGACGCTGTTATTCA | GCACCACTACCCGTC |
| | ATGTGTACGCGCTGC | SHEHFGLLCP | CGCGTCGGGCAAGCA | GGCGGCCGAGCGCA |
| | CGCTCAAGATGCTG | KSIPGLSISGN | GATGTGGCAGGCGCG | AACACCGACACCTGC |
| | AACATCCCCAGCAT | LLMNGQQIFL | TCTCACGGTCTCGGG | CCGTAGCCGACGCTG |
| | CAACGTGCACCACT | EVQAIRETVE | ACTGGCCTGGACGCG | TTATTCACGCGTCGG |
| | ACCCGTCGGCGGCC | LRQYDPVAAL | TCAGCAGAACCAGTG | GCAAGCAGATGTGGC |
| | GAGCGCAAACACCG | FFFDIDLLLQR | GAAAGAGCCCGACGT | AGGCGCGTCTCACGG |
| | ACACCTGCCCGTAG | GPQYSEHPTF | CTACTACACGTCAGC | TCTCGGGACTGGCCT |
| | CCGACGCTGTTATTC | TSQYRIQGKL | GTTCGTGTTTCCCAC | GGACGCGTCAGCAGA |
| | ACGCGTCGGGCAAG | EYRHTWDRH | CAAGGACGTGGCACT | ACCAGTGGAAAGAG |
| | CAGATGTGGCAGGC | DEGAAQGDD | GCGGCACGTGGTGTG | CCCGACGTCTACTAC |
| | GCGTCTCACGGTCTC | DVWTSGSDSD | CGCGCACGAGCTGGT | ACGTCAGCGTTCGTG |
| | GGGACTGGCCTGGA | EELVTTERKT | TTGCTCCATGGAGAA | TTTCCCACCAAGGAC |
| | CGCGTCAGCAGAAC | PRVTGGGAM | CACGCGCGCAACCAA | GTGGCACTGCGGCAC |
| | CAGTGGAAAGAGCC | ASASTSAGRK | GATGCAGGTGATAGG | GTGGTGTGCGCGCAC |
| | CGACGTCTACTACAC | RKSASSATAC | TGACCAGTACGTCAA | GAGCTGGTTTGCTCC |
| | GTCAGCGTTCGTGTT | TAGVMTRGR | GGTGTACCTGGAGTC | ATGGAGAACACGCGC |
| | TCCCACCAAGGACG | LKAESTVAPE | CTTCTGCGAGGACGT | GCAACCAAGATGCAG |
| | TGGCACTGCGGCAC | EDTDEDSDNE | GCCCTCCGGCAAGCT | GTGATAGGTGACCAG |
| | GTGGTGTGCGCGCA | IHNPAVFIWP | CTTTATGCACGTCAC | TACGTCAAGGTGTAC |
| | CGAGCTGGTTTGCTC | PWQAGILARN | GCTGGGCTCTGACGT | CTGGAGTCCTTCTGC |
| | CATGGAGAACACGC | LVPMVATVQ | GGAAGAGGACCTAA | GAGGACGTGCCCTCC |
| | GCGCAACCAAGATG | GQNLKYQEFF | CGATGACCCGCAACC | GGCAAGCTCTTTATG |
| | CAGGTGATAGGTGA | WDANDIYRIF | CGCAACCCTTCATGC | CACGTCACGCTGGGC |
| | CCAGTACGTCAAGG | AELEGVWQP | GCCCCCACGACCGCA | TCTGACGTGGAAGAG |
| | TGTACCTGGAGTCCT | AAQPKRRRHR | ACGGCTTTACGGTGT | GACCTAACGATGACC |
| | TCTGCGAGGACGTG | QDALPGPCIA | TGTGTCCCAAAAATA | CGCAACCCGCAACCC |
| | CCCTCCGGCAAGCTC | STPKKHRGES | TGATAATCAAACCGG | TTCATGCGCCCCCAC |
| | TTTATGCACGTCACG | SAKRKMDPD | GCAAGATCTCGCACA | GAGCGCAACGGCTTT |
| | CTGGGCTCTGACGTG | NPDEGPSSKV | TCATGCTGGATGTGG | ACGGTGTTGTGTCCC |
| | GAAGAGGACCTAAC | PRPETPVTKA | CTTTTACCTCACACG | AAAAATATGATAATC |
| | GATGACCCGCAACC | TTFLQTMLRK | AGCATTTTGGGCTGC | AAACCGGGCAAGATC |
| | CGCAACCCTTCATGC | EVNSQLSLGD | TGTGTCCCAAGAGCA | TCGCACATCATGCTG |
| | GCCCCCACGAGCGC | PLFPELAEESL | TCCCGGGCCTGAGCA | GATGTGGCTTTTACC |
| | AACGGCTTTACGGT | KTFEQVTEDC | TCTCAGGTAACCTGT | TCACACGAGCATTTT |
| | GTTGTGTCCCAAAA | NENPEKDVLT | TGATGAACGGGCAGC | GGGCTGCTGTGTCCC |
| | ATATGATAATCAAA | ELVKQIKVRV | AAATCTTCCTGGAGG | AAGAGCATCCCGGGC |
| | CCGGGCAAGATCTC | DMVRHRIKEH | TAGAAGCGATACGCG | CTGAGCATCTCAGGT |
| | GCACATCATGCTGG | MLKKYTQTEE | AGACCGTGGAACTGC | AACCTGTTGATGAAC |
| | ATGTGGCTTTTACCT | KFTGAFNMM | GTCAGTACGATCCCG | GGGCAGCAAATCTTC |
| | CACACGAGCATTTTG | GGCLQNALDI | TGGCTGCGCTCTTCTT | CTGGAGGTACAAGCG |
| | GGCTGCTGTGTCCCA | LDKVHEPFED | TTTCGATATCGACTT | ATACGCGAGACCGTG |
| | AGAGCATCCCGGGC | MKCIGLTMQS | GTTGCTGCAGCGCGG | GAACTGCGTCAGTAC |
| | CTGAGCATCTCAGGT | MYENYIVPED | GCCTCAGTACAGCGA | GATCCCGTGGCTGCG |
| | AACCTGTTGATGAA | KREMWMACI | GCACCCCACCTTCAC | CTCTTCTTTTTCGATA |
| | CGGGCAGCAAATCT | KELHDVSKGA | CAGCCAGTATCGCAT | TCGACTTGTTGCTGC |
| | TCCTGGAGGTACAA | ANKLGGALQ | CCAGGGCAAGCTTGA | AGCGCGGGCCTCAGT |
| | GCGATACGCGAGAC | AKARAKKDE | GTACCGACACACCTG | ACAGCGAGCACCCCA |
| | CGTGGAACTGCGTC | LRRKMMYMC | GGACCGGCACGACG | CCTTCACCAGCCAGT |
| | AGTACGATCCCGTG | YRNIEFFTKNS | AGGGTGCCGCCCAGG | ATCGCATCCAGGGCA |
| | GCTGCGCTCTTCTTT | AFPKTTNGCS | GCGACGACGACGTCT | AGCTTGAGTACCGAC |
| | TTCGATATCGACTTG | QAMAALQNL | GGACCAGCGGATCGG | ACACCTGGGACCGGC |
| | TTGCTGCAGCGCGG | PQCSPDEIMS | ACTCCGACGAAGAAC | ACGACGAGGGTGCCG |
| | GCCTCAGTACAGCG | YAQKIFKIIDE | TCGTAACCACCGAGC | CCCAGGGCGACGACG |
| | AGCACCCCACCTTCA | ERDKVLTHID | GTAAGACGCCCCGCG | ACGTCTGGACCAGCG |
| | CCAGCCAGTATCGC | HIFMDILTTCV | TCACCGGCGGCGGCG | GATCGGACTCCGACG |
| | ATCCAGGGCAAGCT | ETMCNEYKV | CCATGGCGAGCGCCT | AAGAACTCGTAACCA |
| | TGAGTACCGACACA | TSDACMMTM | CCACTTCCGCGGGCC | CCGAGCGTAAGACGC |
| | CCTGGGACCGGCAC | YGGISLLSEFC | GCAAACGCAAATCAG | CCCGCGTCACCGGCG |
| | GACGAGGGTGCCGC | RVLCCYVLEE | CATCCTCGGCGACGG | GCGGCGCCATGGCGA |
| | CCAGGGCGACGACG | TSVMLAKRPL | CGTGCACGGCGGGCG | GCGCCTCCACTTCCG |
| | ACGTCTGGACCAGC | ITKPEVISVMK | TTATGACACGCGGCC | CGGGCCGCAAACGCA |
| | GGATCGGACTCCGA | RRIEEICMKVF | GCCTTAAGGCCGAGT | AATCAGCATCCTCGG |
| | CGAAGAACTCGTAA | AQYILGADPL | CCACCGTCGCGCCCG | CGACGGCGTGCACGG |
| | CCACCGAGCGTAAG | RVCSPSVDDL | AAGAGGACACCGAC | CGGGCGTTATGACAC |
| | ACGCCCCGCGTCAC | RAIAEESDEEE | GAGGATTCCGACAAC | GCGGCCGCCTTAAGG |
| | CGGCGGCGGCGCCA | AIVAYTLATA | GAAATCCACAATCCG | CCGAGTCCACCGTCG |
| | TGGCGAGCGCCTCC | GASSSDSLVSP | GCCGTGTTCACCTGG | CGCCCGAAGAGGAC |
| | ACTTCCGCGGGCCG | PESPVPATIPL | CCGCCCTGGCAGGCC | ACCGACGAGGATTCC |
| | CAAACGCAAATCAG | SSVIVAENSD | GGCATCCTGGCCCGC | GACAACGAAATCCAC |
| | CATCCTCGGCGACG | QEESEQSDEE | AACCTGGTGCCCATG | AATCCGGCCGTGTTC |
| | GCGTGCACGGCGGG | QEEGAQEERE | GTGGCTACGGTTCAG | ACCTGGCCGCCCTGG |
| | CGTTATGACACGCG | DTVSVKSEPV | GGTCAGAATCTGAAG | CAGGCCGGCATCCTG |
| | GCCGCCTTAAGGCC | SEIEEVASEEE | TACCAGGAGTTCTTC | GCCCGCAACCTGGTG |
| | GAGTCCACCGTCGC | EDGAEEPTAS | TGGGACGCCAACGAC | CCCATGGTGGCTACG |
| | GCCCGAAGAGGACA | GGKSTHPMVT | ATCTACCGCATCTTC | GTTCAGGGTCAGAAT |
| | CCGACGAGGATTCC | RSKADQ (SEQ | GCCGAATTGGAAGGC | CTGAAGTACCAGGAG |
| | GACAACGAAATCCA | ID NO: 73) | GTATGGCAGCCCGCT | TTCTTCTGGGACGCC |
| | CAATCCGGCCGTGTT | | GCGCAACCCAAACGT | AACGACATCTACCGC |
| | CACCTGGCCGCCCTG | | CGCCGCCACCGGCAA | ATCTTCGCCGAATTG |
| | GCAGGCCGGCATCC | | GACGCCTTGCCCGGG | GAAGGCGTATGGCAG |
| | TGGCCCGCAACCTG | | CCATGCATCGCCTCG | CCCGCTGCGCAACCC |
| | GTGCCCATGGTGGCT | | ACGCCCAAAAAGCAC | AAACGTCGCCGCCAC |
| | ACGGTTCAGGGTCA | | CGAGGTGAGTCCTCT | CGGCAAGACGCCTTG |
| | GAATCTGAAGTACC | | GCCAAGAGAAAGAT | CCCGGGCCATGCATC |
| | AGGAGITCLCTGGG | | GGACCCTGATAATCC | GCCTCGACGCCCAAA |
| | ACGCCAACGACATC | | TGACGAGGGCCCTTC | AAGCACCGAGGTGA |
| | TACCGCATCTTCGCC | | CTCCAAGGTGCCACG | GTCCTCTGCCAAGAG |
| | GAATTGGAAGGCGT | | GCCCGAGACACCCGT | AAAGATGGACCCTGA |
| | ATGGCAGCCCGCTG | | GACCAAGGCCACGAC | TAATCCTGACGAGGG |
| | CGCAACCCAAACGT | | GTTCCTGCAGACTAT | CCCTTCCTCCAAGGT |
| | CGCCGCCACCGGCA | | GTTAAGGAAGGAGGT | GCCACGGCCCGAGAC |
| | AGACGCCTTGCCCG | | TAACAGTCAGCTGAG | ACCCGTGACCAAGGC |
| | GGCCATGCATCGCCT | | CCTGGGAGACCCGCT | CACGACGTTCCTGCA |
| | CGACGCCCAAAAAG | | GTTCCCAGAATTGGC | GACTATGTTAAGGAA |
| | CACCGAGGTGAGTC | | CGAAGAATCCCTCAA | GGAGGTTAACAGTCA |
| | CTCTGCCAAGAGAA | | AACCTTTGAACAAGT | GCTGAGCCTGGGAGA |
| | AGATGGACCCTGAT | | GACCGAGGATTGCAA | CCCGCTGTTCCCAGA |
| | AATCCTGACGAGGG | | CGAGAACCCCGAAA | ATTGGCCGAAGAATC |
| | CCCTTCCTCCAAGGT | | AAGATGTCCTGACAG | CCTCAAAACCTTTGA |
| | GCCACGGCCCGAGA | | AACTCGTCAAACAGA | ACAAGTGACCGAGG |
| | CACCCGTGACCAAG | | TTAAGGTTCGAGTGG | ATTGCAACGAGAACC |
| | GCCACGACGTTCCTG | | ACATGGTGCGGCATA | CCGAAAAAGATGTCC |
| | CAGACTATGTTAAG | | GAATCAAGGAGCAC | TGACAGAACTCGTCA |
| | GAAGGAGGTTAACA | | ATGCTGAAAAAATAT | AACAGATTAAGGTTC |
| | GTCAGCTGAGCCTG | | ACCCAGACGGAAGA | GAGTGGACATGGTGC |
| | GGAGACCCGCTGTT | | AAAATTCACTGGCGC | GGCATAGAATCAAGG |
| | CCCAGAATTGGCCG | | CTTTAATATGATGGG | AGCACATGCTGAAAA |
| | AAGAATCCCTCAAA | | AGGATGTTTGCAGAA | AATATACCCAGACGG |
| | ACCTTTGAACAAGT | | TGCCTTAGATATCTT | AAGAAAAATTCACTG |
| | GACCGAGGATTGCA | | AGATAAGGTTCATGA | GCGCCTTTAATATGA |
| | ACGAGAACCCCGAA | | GCCTTTCGAGGACAT | TGGGAGGATGTTTGC |
| | AAAGATGTCCTGAC | | GAAGTGTATTGGGCT | AGAATGCCTTAGATA |
| | AGAACTCGTCAAAC | | AACTATGCAGAGCAT | TCTTAGATAAGGTTC |
| | AGATTAAGGTTCGA | | GTATGAGAACTACAT | ATGAGCCTTTCGAGG |
| | GTGGACATGGTGCG | | TGTACCTGAGGATAA | ACATGAAGTGTATTG |
| | GCATAGAATCAAGG | | GCGGGAGATGTGGAT | GGCTAACTATGCAGA |
| | AGCACATGCTGAAA | | GGCTTGTATTAAGGA | GCATGTATGAGAACT |
| | AAATATACCCAGAC | | GCTGCATGATGTGAG | ACATTGTACCTGAGG |
| | GGAAGAAAAATTCA | | CAAGGGCGCCGCTAA | ATAAGCGGGAGATGT |
| | CTGGCGCCTTTAATA | | CAAGTTGGGGGGTGC | GGATGGCTTGTATTA |
| | TGATGGGAGGATGT | | ACTGCAGGCTAAGGC | AGGAGCTGCATGATG |
| | TTGCAGAATGCCTTA | | CCGTGCTAAAAAGGA | TGAGCAAGGGCGCCG |
| | GATATCTTAGATAA | | TGAACTTAGGAGAAA | CTAACAAGTTGGGGG |
| | GGTTCATGAGCCTTT | | GATGATGTATATGTG | GTGCACTGCAGGCTA |
| | CGAGGACATGAAGT | | CTACAGGAATATAGA | AGGCCCGTGCTAAAA |
| | GTATTGGGCTAACTA | | GTTCTTTACCAAGAA | AGGATGAACTTAGGA |
| | TGCAGAGCATGTAT | | CTCAGCCTTCCCTAA | GAAAGATGATGTATA |
| | GAGAACTACATTGT | | GACCACCAATGGCTG | TGTGCTACAGGAATA |
| | ACCTGAGGATAAGC | | CAGTCAGGCCATGGC | TAGAGTTCTTTACCA |
| | GGGAGATGTGGATG | | GGCATTGCAGAACTT | AGAACTCAGCCTTCC |
| | GCTTGTATTAAGGA | | GCCTCAGTGCTCTCC | CTAAGACCACCAATG |
| | GCTGCATGATGTGA | | TGATGAGATTATGTC | GCTGCAGTCAGGCCA |
| | GCAAGGGCGCCGCT | | TTATGCCCAGAAAAT | TGGCGGCATTGCAGA |
| | AACAAGTTGGGGGG | | CTTTAAGATTTTGGA | ACTTGCCTCAGTGCT |
| | TGCACTGCAGGCTA | | TGAGGAGAGAGACA | CTCCTGATGAGATTA |
| | AGGCCCGTGCTAAA | | AGGTGCTCACGCACA | TGTCTTATGCCCAGA |
| | AAGGATGAACTTAG | | TTGATCACATATTTA | AAATCTTTAAGATTT |
| | GAGAAAGATGATGT | | TGGATATCCTCACTA | TGGATGAGGAGAGA |
| | ATATGTGCTACAGG | | CATGTGTGGAAACAA | GACAAGGTGCTCACG |
| | AATATAGAGTTCTTT | | TGTGTAATGAGTACA | CACATTGATCACATA |
| | ACCAAGAACTCAGC | | AGGTCACTAGTGACG | TTTATGGATATCCTC |
| | CTTCCCTAAGACCAC | | CTTGTATGATGACCA | ACTACATGTGTGGAA |
| | CAATGGCTGCAGTC | | TGTACGGGGGCATCT | ACAATGTGTAATGAG |
| | AGGCCATGGCGGCA | | CTCTCTTAAGTGAGT | TACAAGGTCACTAGT |
| | TTGCAGAACTTGCCT | | TCTGTCGGGTGCTGT | GACGCTTGTATGATG |
| | CAGTGCTCTCCTGAT | | GCTGCTATGTCTTAG | ACCATGTACGGGGGC |
| | GAGATTATGTCTTAT | | AGGAGACTAGTGTGA | ATCTCTCTCTTAAGT |
| | GCCCAGAAAATCTTT | | TGCTGGCCAAGCGGC | GAGTTCTGTCGGGTG |
| | AAGATTTTGGATGA | | CTCTGATAACCAAGC | CTGTGCTGCTATGTC |
| | GGAGAGAGACAAGG | | CTGAGGTTATCAGTG | TTAGAGGAGACTAGT |
| | TGCTCACGCACATTG | | TAATGAAGCGCCGCA | GTGATGCTGGCCAAG |
| | ATCACATATTTATGG | | TTGAGGAGATCTGCA | CGGCCTCTGATAACC |
| | ATATCCTCACTACAT | | TGAAGGTCTTTGCCC | AAGCCTGAGGTTATC |
| | GTGTGGAAACAATG | | AGTACATTCTGGGGG | AGTGTAATGAAGCGC |
| | TGTAATGAGTACAA | | CCGATCCTTTGAGAG | CGCATTGAGGAGATC |
| | GGTCACTAGTGACG | | TCTGCTCTCCTAGTGT | TGCATGAAGGTCTTT |
| | CTTGTATGATGACCA | | GGATGACCTACGGGC | GCCCAGTACATTCTG |
| | TGTACGGGGGCATC | | CATCGCCGAGGAGTC | GGGGCCGATCCTTTG |
| | TCTCTCTTAAGTGAG | | AGATGAGGAAGAGG | AGAGTCTGCTCTCCT |
| | TTCTGTCGGGTGCTG | | CTATTGTAGCCTACA | AGTGTGGATGACCTA |
| | TGCTGCTATGTCTTA | | CTTTGGCCACCGCTG | CGGGCCATCGCCGAG |
| | GAGGAGACTAGTGT | | GTGCCAGCTCCTCTG | GAGTCAGATGAGGA |
| | GATGCTGGCCAAGC | | ATTCTCTGGTGTCAC | AGAGGCTATTGTAGC |
| | GGCCTCTGATAACC | | CTCCAGAGTCCCCTG | CTACACTTTGGCCAC |
| | AAGCCTGAGGTTAT | | TACCCGCGACTATCC | CGCTGGTGCCAGCTC |
| | CAGTGTAATGAAGC | | CTCTGTCCTCAGTAA | CTCTGATTCTCTGGT |
| | GCCGCATTGAGGAG | | TTGTGGCTGAGAACA | GTCACCTCCAGAGTC |
| | ATCTGCATGAAGGT | | GTGATCAGGAAGAA | CCCTGTACCCGCGAC |
| | CTTTGCCCAGTACAT | | AGTGAACAGAGTGAT | TATCCCTCTGTCCTCA |
| | TCTGGGGGCCGATC | | GAGGAACAGGAGGA | GTAATTGTGGCTGAG |
| | CTTTGAGAGTCTGCT | | GGGTGCTCAGGAGGA | AACAGTGATCAGGAA |
| | CTCCTAGTGTGGATG | | GCGGGAGGACACTGT | GAAAGTGAACAGAG |
| | ACCTACGGGCCATC | | GTCTGTCAAGTCTGA | TGATGAGGAACAGG |
| | GCCGAGGAGTCAGA | | GCCAGTGTCTGAGAT | AGGAGGGTGCTCAGG |
| | TGAGGAAGAGGCTA | | AGAGGAAGTTGCCTC | AGGAGCGGGAGGAC |
| | TTGTAGCCTACACTT | | AGAGGAAGAGGAGG | ACTGTGTCTGTCAAG |
| | TGGCCACCGCTGGT | | ATGGTGCTGAGGAAC | TCTGAGCCAGTGTCT |
| | GCCAGCTCCTCTGAT | | CCACCGCCTCTGGAG | GAGATAGAGGAAGTT |
| | TCTCTGGTGTCACCT | | GCAAGAGCACCCACC | GCCTCAGAGGAAGA |
| | CCAGAGTCCCCTGTA | | CTATGGTGACTAGAA | GGAGGATGGTGCTGA |
| | CCCGCGACTATCCCT | | GCAAGGCTGACCAG | GGAACCCACCGCCTC |
| | CTGTCCTCAGTAATT | | (SEQ ID NO: 74) | TGGAGGCAAGAGCA |
| | GTGGCTGAGAACAG | | | CCCACCCTATGGTGA |
| | TGATCAGGAAGAAA | | | CTAGAAGCAAGGCTG |
| | GTGAACAGAGTGAT | | | ACCAGTGATAATAGG |
| | GAGGAACAGGAGGA | | | CTGGAGCCTCGGTGG |
| | GGGTGCTCAGGAGG | | | CCATGCTTCTTGCCC |
| | AGCGGGAGGACACT | | | CTTGGGCCTCCCCCC |
| | GTGTCTGTCAAGTCT | | | AGCCCCTCCTCCCCT |
| | GAGCCAGTGTCTGA | | | TCCTGCACCCGTACC |
| | GATAGAGGAAGTTG | | | CCCGTGGTCTTTGAA |
| | CCTCAGAGGAAGAG | | | TAAAGTCTGAGTGGG |
| | GAGGATGGTGCTGA | | | CGGCAAAAAAAAAA |
| | GGAACCCACCGCCT | | | AAAAAAAAAAAAAA |
| | CTGGAGGCAAGAGC | | | AAAAAAAAAAAAAA |
| | ACCCACCCTATGGTG | | | AAAAAAAAAAAAAA |
| | ACTAGAAGCAAGGC | | | AAAAAAAAAAAAAA |
| | TGACCAGTGATAAT | | | AAAAAAAAAAAAAA |
| | AGGCTGGAGCCTCG | | | AAAAAAAAAAAAAA |
| | GTGGCCATGCTTCTT | | | AAAAAATCTAG (SEQ |
| | GCCCCTTGGGCCTCC | | | ID NO: 75) |
| | CCCCAGCCCCTCCTC | | | |
| | CCCTTCCTGCACCCG | | | |
| | TACCCCCGTGGTCTT | | | |
| | TGAATAAAGTCTGA | | | |
| | GTGGGCGGC (SEQ ID | | | |
| | NO: 72) | | | |
| pp65-IE1 | TCAAGCTTTTGGACC | MESRGRRCPE | ATGGAGTCGCGCGGT | G*GGGAAATAAGAGA |
| mut_DX | CTCGTACAGAAGCT | MISVLGPISGH | CGCCGTTGTCCCGAA | GAAAAGAAGAGTAA |
| | AATACGACTCACTAT | VLKAVFSRGD | ATGATATCCGTACTG | GAAGAAATATAAGA |
| | AGGGAAATAAGAGA | TPVLPHETRL | GGTCCCATTTCGGGT | GCCACCATGGAGTCG |
| | GAAAAGAAGAGTAA | LQTGIHVRVS | CATGTGCTGAAAGCG | CGCGGTCGCCGTTGT |
| | GAAGAAATATAAGA | QPSLILVSQYT | GTGTTTAGTCGCGGC | CCCGAAATGATATCC |
| | GCCACCATGGAGTC | PDSTPCHRGD | GATACGCCAGTACTG | GTACTGGGTCCCATT |
| | GCGCGGTCGCCGTT | NQLQVQHTYF | CCGCACGAGACGCGA | TCGGGTCATGTGCTG |
| | GTCCCGAAATGATA | TGSEVENVSV | CTCCTGCAGACAGGT | AAAGCGGTGTTTAGT |
| | TCCGTACTGGGTCCC | NVHNPTGRSI | ATCCACGTACGCGTG | CGCGGCGATACGCCA |
| | ATTTCGGGTCATGTG | CPSQEPMSIY | AGCCAGCCCTCGCTC | GTACTGCCGCACGAG |
| | CTGAAAGCGGTGTTT | VYALPLKML | ATCCTGGTGTCGCAG | ACGCGACTCCTGCAG |
| | AGTCGCGGCGATAC | NIPSINVHHYP | TACACGCCCGACTCG | ACAGGTATCCACGTA |
| | GCCAGTACTGCCGC | SAAERKHRHL | ACGCCATGCCACCGC | CGCGTGAGCCAGCCC |
| | ACGAGACGCGACTC | PVADAVIHAS | GGCGACAATCAGCTG | TCGCTCATCCTGGTG |
| | CTGCAGACAGGTAT | GKQMWQARL | CAGGTGCAGCACACG | TCGCAGTACACGCCC |
| | CCACGTACGCGTGA | TVSGLAWTR | TACTTTACGGGCAGC | GACTCGACGCCATGC |
| | GCCAGCCCTCGCTCA | QQNQWKEPD | GAGGTGGAGAACGT | CACCGCGGCGACAAT |
| | TCCTGGTGTCGCAGT | VYYTSAFVFP | GTCGGTCAACGTCCA | CAGCTGCAGGTGCAG |
| | ACACGCCCGACTCG | TKDVALRHV | CAACCCCACGGGTCG | CACACGTACTTTACG |
| | ACGCCATGCCACCG | VCAHELVCS | AAGCATCTGCCCCTC | GGCAGCGAGGTGGA |
| | CGGCGACAATCAGC | MENTRATKM | TCAAGAGCCCATGTC | GAACGTGTCGGTCAA |
| | TGCAGGTGCAGCAC | QVIGDQYVKV | GATCTATGTGTACGC | CGTCCACAACCCCAC |
| | ACGTACTTTACGGGC | YLESFCEDVP | GCTGCCGCTCAAGAT | GGGTCGAAGCATCTG |
| | AGCGAGGTGGAGAA | SGKLFMHVTL | GCTGAACATCCCGAG | CCCCTCTCAAGAGCC |
| | CGTGTCGGTCAACGT | GSDVEEDLTM | CATCAACGTGCACCA | CATGTCGATCTATGT |
| | CCACAACCCCACGG | TRNPQPFMRP | CTACCCGAGCGCGGC | GTACGCGCTGCCGCT |
| | GTCGAAGCATCTGC | HERNGFTVLC | CGAGCGCAAACACCG | CAAGATGCTGAACAT |
| | CCCTCTCAAGAGCCC | PKNMIIKPGKI | ACACCTGCCCGTAGC | CCCGAGCATCAACGT |
| | ATGTCGATCTATGTG | SHIMLDVAFT | CGACGCTGTTATTCA | GCACCACTACCCGAG |
| | TACGCGCTGCCGCTC | SHEHFGLLCP | CGCGTCGGGCAAGCA | CGCGGCCGAGCGCAA |
| | AAGATGCTGAACAT | KSIPGLSISGN | GATGTGGCAAGCGCG | ACACCGACACCTGCC |
| | CCCGAGCATCAACG | LLMNGQQIFL | CCTCACGGTCTCGGG | CGTAGCCGACGCTGT |
| | TGCACCACTACCCG | EVQAIRETVE | ACTAGCCTGGACGCG | TATTCACGCGTCGGG |
| | AGCGCGGCCGAGCG | LRQYDPVAAL | TCAGCAGAACCAGTG | CAAGCAGATGTGGCA |
| | CAAACACCGACACC | FFFDIDLLLQR | GAAAGAGCCCGACGT | AGCGCGCCTCACGGT |
| | TGCCCGTAGCCGAC | GPQYSEHPTF | CTACTACACGTCAGC | CTCGGGACTAGCCTG |
| | GCTGTTATTCACGCG | TSQYRIQGKL | GTTCGTGTTTCCCAC | GACGCGTCAGCAGAA |
| | TCGGGCAAGCAGAT | EYRHTWDRH | CAAGGACGTGGCACT | CCAGTGGAAAGAGCC |
| | GTGGCAAGCGCGCC | DEGAAQGDD | GCGCCACGTGGTGTG | CGACGTCTACTACAC |
| | TCACGGTCTCGGGA | DVWTSGSDSD | TGCGCACGAGCTGGT | GTCAGCGTTCGTGTT |
| | CTAGCCTGGACGCG | EELVTTERKT | TTGCTCCATGGAGAA | TCCCACCAAGGACGT |
| | TCAGCAGAACCAGT | PRVTGGGAM | TACGCGCGCAACCAA | GGCACTGCGCCACGT |
| | GGAAAGAGCCCGAC | ASASTSAGSA | GATGCAGGTGATAGG | GGTGTGTGCGCACGA |
| | GTCTACTACACGTCA | SSATACTAGV | TGATCAATACGTCAA | GCTGGTTTGCTCCAT |
| | GCGTTCGTGTTTCCC | MTRGRLKAES | GGTGTACCTGGAGTC | GGAGAATACGCGCGC |
| | ACCAAGGACGTGGC | TVAPEEDTDE | CTTCTGCGAGGATGT | AACCAAGATGCAGGT |
| | ACTGCGCCACGTGG | DSDNEIHNPA | GCCCTCCGGTAAGCT | GATAGGTGATCAATA |
| | TGTGTGCGCACGAG | VFTWPPWQA | CTTTATGCACGTCAC | CGTCAAGGTGTACCT |
| | CTGGTTTGCTCCATG | GILARNLVPM | GCTGGGCTCTGACGT | GGAGTCCTTCTGCGA |
| | GAGAATACGCGCGC | VATVQGQNL | GGAAGAGGACCTAA | GGATGTGCCCTCCGG |
| | AACCAAGATGCAGG | KYQEFFWDA | CGATGACCCGCAATC | TAAGCTCTTTATGCA |
| | TGATAGGTGATCAA | NDIYRIFAELE | CGCAACCCTTCATGC | CGTCACGCTGGGCTC |
| | TACGTCAAGGTGTA | GVWQPAAQP | GCCCCCACGAGCGCA | TGACGTGGAAGAGG |
| | CCTGGAGTCCTTCTG | KRRRHRQDA | ACGGCTTTACGGTGT | ACCTAACGATGACCC |
| | CGAGGATGTGCCCT | LPGPCIASTPK | TGTGTCCTAAAAATA | GCAATCCGCAACCCT |
| | CCGGTAAGCTCTTTA | KHRGESSAKR | TGATAATCAAACCAG | TCATGCGCCCCCACG |
| | TGCACGTCACGCTG | KMDPDNPDE | GCAAGATCTCGCACA | AGCGCAACGGCTTTA |
| | GGCTCTGACGTGGA | GPSSKVPRPET | TCATGCTGGATGTGG | CGGTGTTGTGTCCTA |
| | AGAGGACCTAACGA | PVTKATTFLQ | CTTTTACCTCACACG | AAAATATGATAATCA |
| | TGACCCGCAATCCG | TMLRKEVNSQ | AGCATTTTGGGCTGC | AACCAGGCAAGATCT |
| | CAACCCTTCATGCGC | LSLGDPLFPEL | TGTGTCCCAAGAGCA | CGCACATCATGCTGG |
| | CCCCACGAGCGCAA | AEESLKTFEQ | TCCCGGGCCTGAGCA | ATGTGGCTTTTACCT |
| | CGGCTTTACGGTGTT | VTEDCNENPE | TCTCAGGTAACCTGT | CACACGAGCATTTTG |
| | GTGTCCTAAAAATAT | KDVLTELVKQ | TGATGAACGGGCAGC | GGCTGCTGTGTCCCA |
| | GATAATCAAACCAG | IKVRVDMVR | AAATCTTTCTCGAGG | AGAGCATCCCGGGCC |
| | GCAAGATCTCGCAC | HRIKEHMLKK | TGCAAGCTATACGCG | TGAGCATCTCAGGTA |
| | ATCATGCTGGATGTG | YTQTEEKFTG | AGACCGTCGAACTGC | ACCTGTTGATGAACG |
| | GCTTTTACCTCACAC | AFNMMGGCL | GTCAGTACGATCCCG | GGCAGCAAATCTTTC |
| | GAGCATTTTGGGCTG | QNALDILDKV | TGGCTGCGCTGTTCT | TCGAGGTGCAAGCTA |
| | CTGTGTCCCAAGAG | HEPFEDMKCI | TTTTCGATATCGACTT | TACGCGAGACCGTCG |
| | CATCCCGGGCCTGA | GLTMQSMYE | GTTGCTGCAGCGCGG | AACTGCGTCAGTACG |
| | GCATCTCAGGTAAC | NYIVPEDKRE | GCCTCAGTACAGCGA | ATCCCGTGGCTGCGC |
| | CTGTTGATGAACGG | MWMACIKEL | GCACCCCACCTTCAC | TGTTCTTTTTCGATAT |
| | GCAGCAAATCTTTCT | HDVSKGAAN | CAGCCAGTATCGCAT | CGACTTGTTGCTGCA |
| | CGAGGTGCAAGCTA | KLGGALQAK | CCAGGGCAAGCTTGA | GCGCGGGCCTCAGTA |
| | TACGCGAGACCGTC | ARAKKDELRR | GTACCGACACACCTG | CAGCGAGCACCCCAC |
| | GAACTGCGTCAGTA | KMMYMCYR | GGACCGGCACGACG | CTTCACCAGCCAGTA |
| | CGATCCCGTGGCTGC | NIEHIKNSAF | AGGGAGCCGCCCAG | TCGCATCCAGGGCAA |
| | GCTGTTCTTTTTCGA | PKTTNGCSQA | GGCGACGACGACGTC | GCTTGAGTACCGACA |
| | TATCGACTTGTTGCT | MAALQNLPQ | TGGACCTCTGGATCG | CACCTGGGACCGGCA |
| | GCAGCGCGGGCCTC | CSPDEIMSYA | GACTCCGACGAAGAA | CGACGAGGGAGCCG |
| | AGTACAGCGAGCAC | QKIFKILDEER | CTCGTAACGACCGAG | CCCAGGGCGACGACG |
| | CCCACCTTCACCAGC | DKVLTHIDHIF | CGTAAGACCCCCCGC | ACGTCTGGACCTCTG |
| | CAGTATCGCATCCA | MDILTTCVET | GTCACCGGCGGCGGC | GATCGGACTCCGACG |
| | GGGCAAGCTTGAGT | MCNEYKVTS | GCCATGGCGTCCGCC | AAGAACTCGTAACGA |
| | ACCGACACACCTGG | DACMMTMYG | TCAACTTCCGCGGGC | CCGAGCGTAAGACCC |
| | GACCGGCACGACGA | GISLLSEFCRV | TCAGCATCCTCGGCT | CCCGCGTCACCGGCG |
| | GGGAGCCGCCCAGG | LCCYVLEETS | ACGGCGTGCACGGCG | GCGGCGCCATGGCGT |
| | GCGACGACGACGTC | VMLAKRPLIT | GGCGTTATGACACGT | CCGCCTCAACTTCCG |
| | TGGACCTCTGGATCG | KPEVISVMKR | GGCAGACTTAAGGCC | CGGGCTCAGCATCCT |
| | GACTCCGACGAAGA | RIEEICMKVFA | GAGTCCACCGTCGCG | CGGCTACGGCGTGCA |
| | ACTCGTAACGACCG | QYILGADPLR | CCCGAAGAGGACACC | CGGCGGGCGTTATGA |
| | AGCGTAAGACCCCC | VCSPSVDDLR | GACGAGGATTCCGAC | CACGTGGCAGACTTA |
| | CGCGTCACCGGCGG | AIAEESDEEEA | AACGAAATCCACAAT | AGGCCGAGTCCACCG |
| | CGGCGCCATGGCGT | IVAYTLATAG | CCGGCCGTGTTCACC | TCGCGCCCGAAGAGG |
| | CCGCCTCAACTTCCG | ASSSDSLVSPP | TGGCCGCCCTGGCAG | ACACCGACGAGGATT |
| | CGGGCTCAGCATCCT | ESPVPATIPLS | GCCGGCATCCTGGCC | CCGACAACGAAATCC |
| | CGGCTACGGCGTGC | SVIVAENSDQ | CGCAACCTGGTGCCC | ACAATCCGGCCGTGT |
| | ACGGCGGGCGTTAT | EESEQSDEEQ | ATGGTGGCTACGGTT | TCACCTGGCCGCCCT |
| | GACACGTGGCAGAC | EEGAQEERED | CAGGGTCAGAATCTG | GGCAGGCCGGCATCC |
| | TTAAGGCCGAGTCC | TVSVKSEPVS | AAGTACCAGGAGTTC | TGGCCCGCAACCTGG |
| | ACCGTCGCGCCCGA | EIEEVASEEEE | TTCTGGGACGCCAAC | TGCCCATGGTGGCTA |
| | AGAGGACACCGACG | DGAEEPTASG | GACATCTACCGCATC | CGGTTCAGGGTCAGA |
| | AGGATTCCGACAAC | GKSTHPMVTR | TTCGCCGAATTGGAA | ATCTGAAGTACCAGG |
| | GAAATCCACAATCC | SKADQ (SEQ | GGCGTATGGCAGCCC | AGTTCTTCTGGGACG |
| | GGCCGTGTTCACCTG | ID NO: 77) | GCTGCGCAACCCAAA | CCAACGACATCTACC |
| | GCCGCCCTGGCAGG | | CGTCGCCGCCACCGG | GCATCTTCGCCGAAT |
| | CCGGCATCCTGGCCC | | CAAGACGCCTTGCCC | TGGAAGGCGTATGGC |
| | GCAACCTGGTGCCC | | GGGCCATGCATCGCC | AGCCCGCTGCGCAAC |
| | ATGGTGGCTACGGTT | | TCGACGCCCAAAAAG | CCAAACGTCGCCGCC |
| | CAGGGTCAGAATCT | | CACCGAGGTGAGTCC | ACCGGCAAGACGCCT |
| | GAAGTACCAGGAGT | | TCTGCCAAGAGAAAG | TGCCCGGGCCATGCA |
| | TCTTCTGGGACGCCA | | ATGGACCCTGATAAT | TCGCCTCGACGCCCA |
| | ACGACATCTACCGC | | CCTGACGAGGGCCCT | AAAAGCACCGAGGT |
| | ATCTTCGCCGAATTG | | TCCTCCAAGGTGCCA | GAGTCCTCTGCCAAG |
| | GAAGGCGTATGGCA | | CGGCCCGAGACACCC | AGAAAGATGGACCCT |
| | GCCCGCTGCGCAAC | | GTGACCAAGGCCACG | GATAATCCTGACGAG |
| | CCAAACGTCGCCGC | | ACGTTCCTGCAGACT | GGCCCTTCCTCCAAG |
| | CACCGGCAAGACGC | | ATGTTAAGGAAGGAG | GTGCCACGGCCCGAG |
| | CTTGCCCGGGCCATG | | GTTAACAGTCAGCTG | ACACCCGTGACCAAG |
| | CATCGCCTCGACGCC | | AGCCTGGGAGACCCG | GCCACGACGTTCCTG |
| | CAAAAAGCACCGAG | | CTGTTCCCAGAATTG | CAGACTATGTTAAGG |
| | GTGAGTCCTCTGCCA | | GCCGAAGAATCCCTC | AAGGAGGTTAACAGT |
| | AGAGAAAGATGGAC | | AAAACCTTTGAACAA | CAGCTGAGCCTGGGA |
| | CCTGATAATCCTGAC | | GTGACCGAGGATTGC | GACCCGCTGTTCCCA |
| | GAGGGCCCTTCCTCC | | AACGAGAACCCCGA | GAATTGGCCGAAGAA |
| | AAGGTGCCACGGCC | | AAAAGATGTCCTGAC | TCCCTCAAAACCTTT |
| | CGAGACACCCGTGA | | AGAACTCGTCAAACA | GAACAAGTGACCGA |
| | CCAAGGCCACGACG | | GATTAAGGTTCGAGT | GGATTGCAACGAGAA |
| | TTCCTGCAGACTATG | | GGACATGGTGCGGCA | CCCCGAAAAAGATGT |
| | TTAAGGAAGGAGGT | | TAGAATCAAGGAGCA | CCTGACAGAACTCGT |
| | TAACAGTCAGCTGA | | CATGCTGAAAAAATA | CAAACAGATTAAGGT |
| | GCCTGGGAGACCCG | | TACCCAGACGGAAGA | TCGAGTGGACATGGT |
| | CTGTTCCCAGAATTG | | AAAATTCACTGGCGC | GCGGCATAGAATCAA |
| | GCCGAAGAATCCCT | | CTTTAATATGATGGG | GGAGCACATGCTGAA |
| | CAAAACCTTTGAAC | | AGGATGTTTGCAGAA | AAAATATACCCAGAC |
| | AAGTGACCGAGGAT | | TGCCTTAGATATCTT | GGAAGAAAAATTCAC |
| | TGCAACGAGAACCC | | AGATAAGGTTCATGA | TGGCGCCTTTAATAT |
| | CGAAAAAGATGTCC | | GCCTTTCGAGGACAT | GATGGGAGGATGTTT |
| | TGACAGAACTCGTC | | GAAGTGTATTGGGCT | GCAGAATGCCTTAGA |
| | AAACAGATTAAGGT | | AACTATGCAGAGCAT | TATCTTAGATAAGGT |
| | TCGAGTGGACATGG | | GTATGAGAACTACAT | TCATGAGCCTTTCGA |
| | TGCGGCATAGAATC | | TGTACCTGAGGATAA | GGACATGAAGTGTAT |
| | AAGGAGCACATGCT | | GCGGGAGATGTGGAT | TGGGCTAACTATGCA |
| | GAAAAAATATACCC | | GGCTTGTATTAAGGA | GAGCATGTATGAGAA |
| | AGACGGAAGAAAAA | | GCTGCATGATGTGAG | CTACATTGTACCTGA |
| | TTCACTGGCGCCTTT | | CAAGGGCGCCGCTAA | GGATAAGCGGGAGA |
| | AATATGATGGGAGG | | CAAGTTGGGGGGTGC | TGTGGATGGCTTGTA |
| | ATGTTTGCAGAATGC | | ACTGCAGGCTAAGGC | TTAAGGAGCTGCATG |
| | CTTAGATATCTTAGA | | CCGTGCTAAAAAGGA | ATGTGAGCAAGGGCG |
| | TAAGGTTCATGAGC | | TGAACTTAGGAGAAA | CCGCTAACAAGTTGG |
| | CTTTCGAGGACATG | | GATGATGTATATGTG | GGGGTGCACTGCAGG |
| | AAGTGTATTGGGCT | | CTACAGGAATATAGA | CTAAGGCCCGTGCTA |
| | AACTATGCAGAGCA | | GTTCTTTACCAAGAA | AAAAGGATGAACTTA |
| | TGTATGAGAACTAC | | CTCAGCCTTCCCTAA | GGAGAAAGATGATGT |
| | ATTGTACCTGAGGAT | | GACCACCAATGGCTG | ATATGTGCTACAGGA |
| | AAGCGGGAGATGTG | | CTCGCAGGCCATGGC | ATATAGAGTTCTTTA |
| | GATGGCTTGTATTAA | | GGCATTGCAGAACTT | CCAAGAACTCAGCCT |
| | GGAGCTGCATGATG | | GCCTCAGTGCTCTCC | TCCCTAAGACCACCA |
| | TGAGCAAGGGCGCC | | TGATGAGATTATGTC | ATGGCTGCTCGCAGG |
| | GCTAACAAGTTGGG | | TTATGCCCAGAAAAT | CCATGGCGGCATTGC |
| | GGGTGCACTGCAGG | | CTTTAAGATTTTGGA | AGAACTTGCCTCAGT |
| | CTAAGGCCCGTGCT | | TGAGGAGCGAGACA | GCTCTCCTGATGAGA |
| | AAAAAGGATGAACT | | AGGTGCTTACGCACA | TTATGTCTTATGCCC |
| | TAGGAGAAAGATGA | | TTGATCACATATTTA | AGAAAATCTTTAAGA |
| | TGTATATGTGCTACA | | TGGATATCCTCACTA | TTTTGGATGAGGAGC |
| | GGAATATAGAGTTC | | CATGTGTTGAAACGA | GAGACAAGGTGCTTA |
| | TTTACCAAGAACTCA | | TGTGCAATGAGTACA | CGCACATTGATCACA |
| | GCCTTCCCTAAGACC | | AGGTCACTAGTGACG | TATTTATGGATATCC |
| | ACCAATGGCTGCTC | | CTTGTATGATGACCA | TCACTACATGTGTTG |
| | GCAGGCCATGGCGG | | TGTACGGGGGCATAT | AAACGATGTGCAATG |
| | CATTGCAGAACTTGC | | CTCTCTTAAGTGAAT | AGTACAAGGTCACTA |
| | CTCAGTGCTCTCCTG | | TCTGTCGGGTGCTGT | GTGACGCTTGTATGA |
| | ATGAGATTATGTCTT | | GCTGCTACGTCTTAG | TGACCATGTACGGGG |
| | ATGCCCAGAAAATC | | AGGAGACTAGTGTGA | GCATATCTCTCTTAA |
| | TTTAAGATTTTGGAT | | TGCTGGCCAAGCGGC | GTGAATTCTGTCGGG |
| | GAGGAGCGAGACAA | | CTCTGATAACCAAGC | TGCTGTGCTGCTACG |
| | GGTGCTTACGCACAT | | CTGAGGTCATCAGTG | TCTTAGAGGAGACTA |
| | TGATCACATATTTAT | | TAATGAAGCGCCGCA | GTGTGATGCTGGCCA |
| | GGATATCCTCACTAC | | TTGAGGAGATCTGCA | AGCGGCCTCTGATAA |
| | ATGTGTTGAAACGA | | TGAAGGTCTTTGCCC | CCAAGCCTGAGGTCA |
| | TGTGCAATGAGTAC | | AGTACATTCTGGGGG | TCAGTGTAATGAAGC |
| | AAGGTCACTAGTGA | | CCGATCCTTTGAGAG | GCCGCATTGAGGAGA |
| | CGCTTGTATGATGAC | | TCTGCTCTCCAAGTG | TCTGCATGAAGGTCT |
| | CATGTACGGGGGCA | | TGGATGACCTACGGG | TTGCCCAGTACATTC |
| | TATCTCTCTTAAGTG | | CCATCGCCGAGGAGT | TGGGGGCCGATCCTT |
| | AATTCTGTCGGGTGC | | CAGACGAGGAAGAG | TGAGAGTCTGCTCTC |
| | TGTGCTGCTACGTCT | | GCTATTGTAGCCTAC | CAAGTGTGGATGACC |
| | TAGAGGAGACTAGT | | ACTTTGGCCACCGCT | TACGGGCCATCGCCG |
| | GTGATGCTGGCCAA | | GGTGCCAGCTCCTCT | AGGAGTCAGACGAG |
| | GCGGCCTCTGATAA | | GACTCTCTGGTGTCA | GAAGAGGCTATTGTA |
| | CCAAGCCTGAGGTC | | CCTCCAGAATCCCCT | GCCTACACTTTGGCC |
| | ATCAGTGTAATGAA | | GTGCCCGCGACAATC | ACCGCTGGTGCCAGC |
| | GCGCCGCATTGAGG | | CCTCTGTCCTCAGTA | TCCTCTGACTCTCTG |
| | AGATCTGCATGAAG | | ATTGTGGCTGAGAAC | GTGTCACCTCCAGAA |
| | GTCTTTGCCCAGTAC | | AGTGATCAGGAAGA | TCCCCTGTGCCCGCG |
| | ATTCTGGGGGCCGA | | AAGTGAACAGAGTG | ACAATCCCTCTGTCC |
| | TCCTTTGAGAGTCTG | | ATGAGGAACAGGAG | TCAGTAATTGTGGCT |
| | CTCTCCAAGTGTGGA | | GAGGGTGCTCAGGAG | GAGAACAGTGATCAG |
| | TGACCTACGGGCCA | | GAGCGGGAGGATACT | GAAGAAAGTGAACA |
| | TCGCCGAGGAGTCA | | GTGTCTGTCAAGTCT | GAGTGATGAGGAAC |
| | GACGAGGAAGAGGC | | GAGCCAGTGTCTGAA | AGGAGGAGGGTGCTC |
| | TATTGTAGCCTACAC | | ATTGAGGAAGTTGCC | AGGAGGAGCGGGAG |
| | TTTGGCCACCGCTGG | | TCAGAGGAAGAGGA | GATACTGTGTCTGTC |
| | TGCCAGCTCCTCTGA | | GGATGGTGCTGAGGA | AAGTCTGAGCCAGTG |
| | CTCTCTGGTGTCACC | | ACCCACCGCCTCTGG | TCTGAAATTGAGGAA |
| | TCCAGAATCCCCTGT | | AGGCAAGTCCACCCA | GTTGCCTCAGAGGAA |
| | GCCCGCGACAATCC | | CCCTATGGTAACTAG | GAGGAGGATGGTGCT |
| | CTCTGTCCTCAGTAA | | ATCAAAGGCTGACCA | GAGGAACCCACCGCC |
| | TTGTGGCTGAGAAC | | G (SEQ ID NO: 78) | TCTGGAGGCAAGTCC |
| | AGTGATCAGGAAGA | | | ACCCACCCTATGGTA |
| | AAGTGAACAGAGTG | | | ACTAGATCAAAGGCT |
| | ATGAGGAACAGGAG | | | GACCAGTGATAATAG |
| | GAGGGTGCTCAGGA | | | GCTGGAGCCTCGGTG |
| | GGAGCGGGAGGATA | | | GCCATGCTTCTTGCC |
| | CTGTGTCTGTCAAGT | | | CCTTGGGCCTCCCCC |
| | CTGAGCCAGTGTCTG | | | CAGCCCCTCCTCCCC |
| | AAATTGAGGAAGTT | | | TTCCTGCACCCGTAC |
| | GCCTCAGAGGAAGA | | | CCCCGTGGTCTTTGA |
| | GGAGGATGGTGCTG | | | ATAAAGTCTGAGTGG |
| | AGGAACCCACCGCC | | | GCGGCAAAAAAAAA |
| | TCTGGAGGCAAGTC | | | AAAAAAAAAAAAAA |
| | CACCCACCCTATGGT | | | AAAAAAAAAAAAAA |
| | AACTAGATCAAAGG | | | AAAAAAAAAAAAAA |
| | CTGACCAGTGATAA | | | AAAAAAAAAAAAAA |
| | TAGGCTGGAGCCTC | | | AAAAAAAAAAAAAA |
| | GGTGGCCATGCTTCT | | | AAAAAAAAAAAAAA |
| | TGCCCCTTGGGCCTC | | | AAAAAAATCTAG |
| | CCCCCAGCCCCTCCT | | | (SEQ ID NO: 79) |
| | CCCCTTCCTGCACCC | | | |
| | GTACCCCCGTGGTCT | | | |
| | TTGAATAAAGTCTG | | | |
| | AGTGGGCGGC (SEQ | | | |
| | ID NO: 76) | | | |

TABLE 10
Ratios of gB:Pentamer

| | | | | | | | | | | | | Total |
|---|---|---|---|---|---|---|---|---|---|---|---|---|
| | | | | | | | | | | | Total | Min |
| | | | | | | | | Dose | Dose | Dose | Vol | Mass |
| Test/Control | | | | Dosing | # of | # of | # of | Level | Vol | Conc. | Req | Req |
| Material | Vehicle | Formulation | Route | Regimen | Doses | males | females | (mg/kg) | (mL) | (mg/mL) | (mL) | (mg) |
| Pentamer (3) + gB (7) + | MC3 | 50:10:38:5:1.5; | IM | d0, d21, d141 | 3 | | 5 | 0.6 | 0.05 | 0.24 | 1.05 | 0.252 |
| pp6S-IE1(2) | | 72 ± 1 nm | | | | | | | | | | |
| Pentamer (5) + gB (5) + | MC3 | 50:10:38:5:1.5; | IM | d0, d21, d141 | 3 | | 25 | 0.6 | 0.05 | 0.24 | 5.25 | 1.26 |
| pp6S-IE1(2) | | 72 ± 1 nm | | | | | | | | | | |
| Pentamer (8) + gB(2) + | MC3 | 50:10:38:5:1.5; | IM | d0, d21, d141 | 3 | | 5 | 0.6 | 0.05 | 0.24 | 1.05 | 0.252 |
| pp6S-IE1(2) | | 72 ± 1 nm | | | | | | | | | | |
| Pentamer (10) + gB (2) | MC3 | 50:10:38:5:1.5; | IM | d0, d21, d141 | 3 | | 5 | 0.6 | 0.05 | 0.24 | 1.05 | 0.252 |
| | | 72 ± 1 nm | | | | | | | | | | |
| empty LNP | MC3 | 50:10:38:5:1.5; | IM | d0, d21, d141 | 3 | | 25 | | 0.05 | | | |
| | | 72 ± 1 nm | | | | | | | | | | |
| pp6S-IE3 (2) | MC3 | 50:10:38:5:1.5; | IM | d0, d21 | 3 | | 20 | 0.1 | 0.05 | 0.04 | 4.2 | 0.168 |
| | | 72 ± 1 nm | | | | | | | | | | |

### Example 34: Immunogenicity Study of hCMV mRNA Vaccines Formulated in Compound 25 Lipid Nanoparticles

Different lipid nanoparticle formulations (e.g., cationic lipid formulations) were tested for the delivery of the hCMV mRNA vaccines. hCMV mRNA vaccine constructs encoding the pentamer, gB, and pp65-IE1 were formulated in Compound 25 or MC3 lipid nanoparticles for immunizing Cynomolgus macaques. The dosages and immunization regimen were as indicated in Table 11. All animals in the study were naturally infected with Cynomolgus CMV and had low but varying titers of anti-cyno CMV antibodies. Upon immunization with the hCMV mRNA vaccines, no injection site interactions (Draize score 0) were observed in either Compound 25 or MC3 formulations for all doses. Cynomolgus macaques received 100 μg of total mRNA vaccines in either formulation were monitored for 6 months to evaluate the immunogenicity of the mRNA vaccines and the duration of antibody response.

Serum samples were taken from the immunized animals on days 0, 21, and 42 post immunization. Serum pentamer-specific IgG titers were assayed on pentamer coated plates. Compound 25 and MC3 formulations induced comparable IgG titers at high doses (FIG. 24). The hCMV mRNA vaccines formulated in different lipids were also tested for their ability to induce neutralizing antibodies against hCMV in Cynomolgus. Serum samples were taken on days 0 and 42 for analysis in neutralization assays. The neutralization assays were performed on ARPE-19 epithelial cells infected with hCMV clinical isolate VR1814 strain (FIG. 25A), or HEL299 fibroblast cells infected with hCMV AS169 strain (FIG. 25B). The results showed that a 100 μg total dose of the hCMV pentamer mRNA vaccines formulated in either lipids induced comparable neutralizing antibody titers as CytoGam®, a hyperimmune serum used clinically for hCMV prevention.

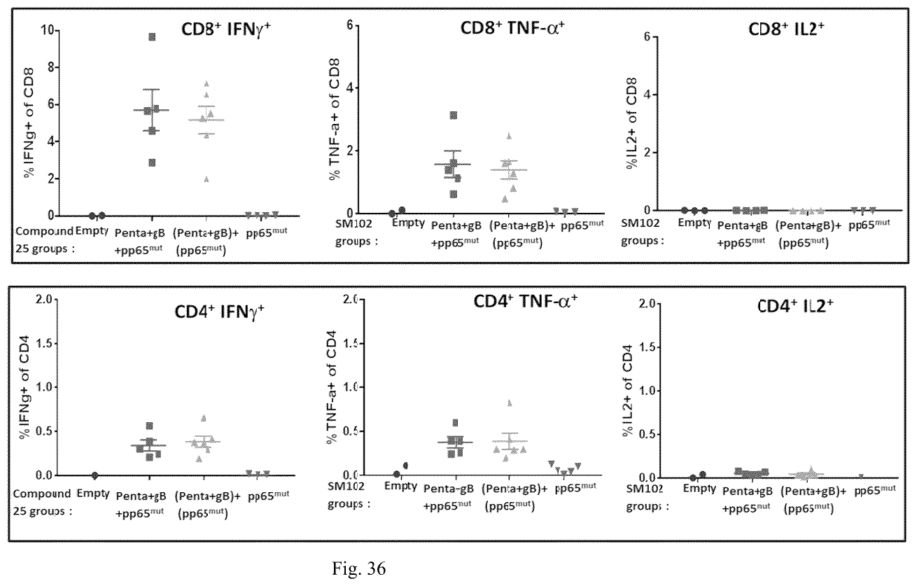

Further, different configurations of the multivalent hCMV mRNA vaccine formulations were tested using the Compound 25 lipids (FIGS. 36A-36B). The results showed that different formulations elicited similar T cell response against pp65 or the pentamer in mice. FIG. 36 shows that the multivalent mRNA vaccine induced robust T cell response to the pentamer.

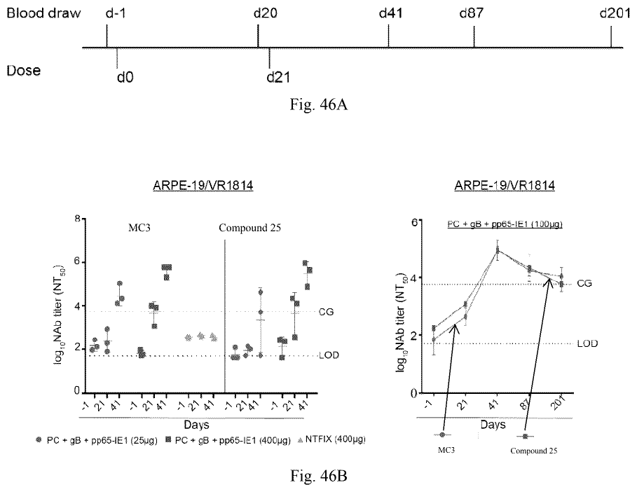

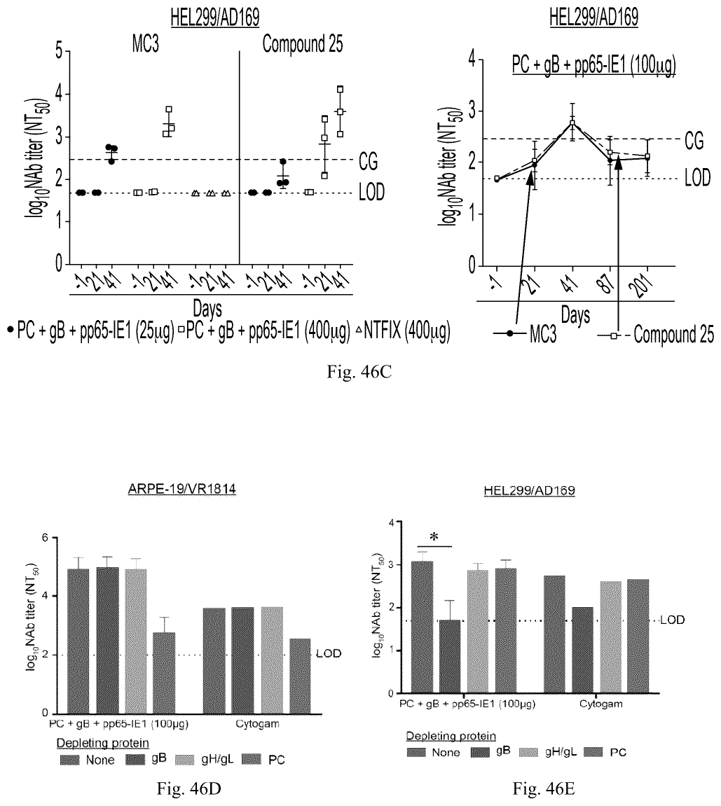

In one embodiment, cynomolgus macaques (cynos) were vaccinated according to the dosing regimen in FIG. 46A with the indicated doses of two different LNPs (MC3 and Compound 25) formulations containing the mRNA constructs encoding the pentameric complex, gB, and fusion of pp65 and IE1 (referred to as pp65-IE1) or a nontranslating Factor IX mRNA (NTFIX) as a control (FIG. 46B). All monkeys used in this study had been naturally exposed to cyno CMV (cyCMV), and the majority had extremely low neutralizing titers against epithelial (NT50<500) but not fibroblast infection by CMV prior to immunization. ELISA results showed high titers of anti-PC and -gB antibodies after vaccination (FIGS. 47A and 47B). A dose-dependent increase in neutralizing antibodies was observed three weeks following the second vaccination. The neutralizing titers were higher than or equivalent to Cytogam in epithelial and fibroblast cells, respectively, at all doses of vaccine (FIGS. 46B and 46C).

Cynos that received a 100 μg dose were monitored for an additional six months following the second dose vaccination. After the second dose, the neutralizing titers against epithelial and fibroblast infection initially dropped three- and eightfold, respectively, but thereafter were sustained for an additional four months (FIGS. 46B and 46C). Overall, vaccination with mRNA formulated in MC3 and Compound 25 elicited equivalent neutralizing titers and therefore further studies utilized Compound 25.

The specificity of these antibodies was further evaluated by performing antibody depletion experiments similar to those done with mouse immune sera. Purified PC and gB protein competed with neutralizing activity of NHP immune sera and Cytogam, in epithelial and fibroblast cells, respectively (FIGS. 46D and 46E), consistent with previous observations (Chandramouli et al., 2015). The results demonstrate that vaccination with CMV mRNA antigens elicits antibody specificities similar to those observed in CMV-seropositive individuals.

TABLE 11
Lipid Formulation Test

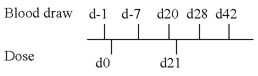

| Group | Test/Control group | vehicle | Route | Dosing Regimen | N | # of doses | Dose (ug) |
|---|---|---|---|---|---|---|---|
| 1 | Pentamer + gB + pp65-IE1 | Compound | IM | d 0, d 21 | 3 | 2 | 400 |
| | | 25 | | | | | |
| 2 | Pentamer + gB + pp65-IE1 | Compound | IM | d 0, d 21 | 3 | 2 | 100 |
| | | 25 | | | | | |
| 3 | Pentamer + gB + pp65-IE1 | Compound | IM | d 0, d 21 | 3 | 2 | 25 |
| | | 25 | | | | | |
| 4 | NTFIX (non translating RNA) | Compound | IM | d 0, d 21 | 3 | 2 | 400 |
| | | 25 | | | | | |
| 5 | Pentamer + gB + pp65-IE1 | MC3 | IM | d 0, d 21 | 3 | 2 | 400 |
| 6 | Pentamer + gB + pp65-IE1 | MC3 | IM | d 0, d 21 | 3 | 2 | 100 |
| 7 | Pentamer + gB + pp65-IE1 | MC3 | IM | d 0, d 21 | 3 | 2 | 25 |

### Example 35: Evaluation of Immunogenicity of hCMV mRNA Vaccine Constructs with or without Chemical Modification and in Different Lipid Formulations

The immunogenicity of hCMV mRNA vaccine constructs with or without chemical modification and in different lipid formulations was evaluated. hCMV mRNA vaccines encoding hCMV pentamer (5 μg) and gB (1 μg), or constructs encoding the hCMV pentamer (5 μg), gB (1 μg), and pp65mut (2 μg) were formulated in either MC3 lipid particles or compound 25 lipid particles. Balb/C mice were immunized with two doses (one primary dose and one booster dose 21 days after the primary dose) of the hCMV mRNA vaccines and sera were collected at days 21 and 43 post primary dose.

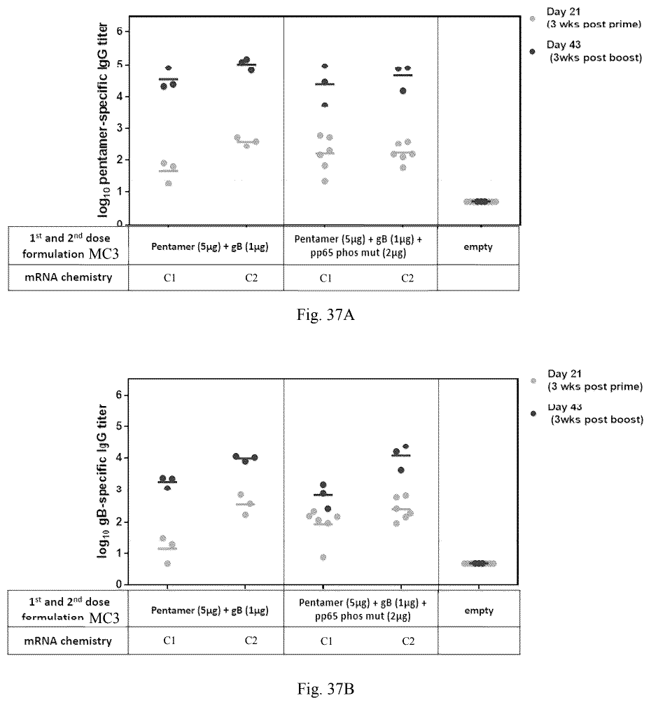

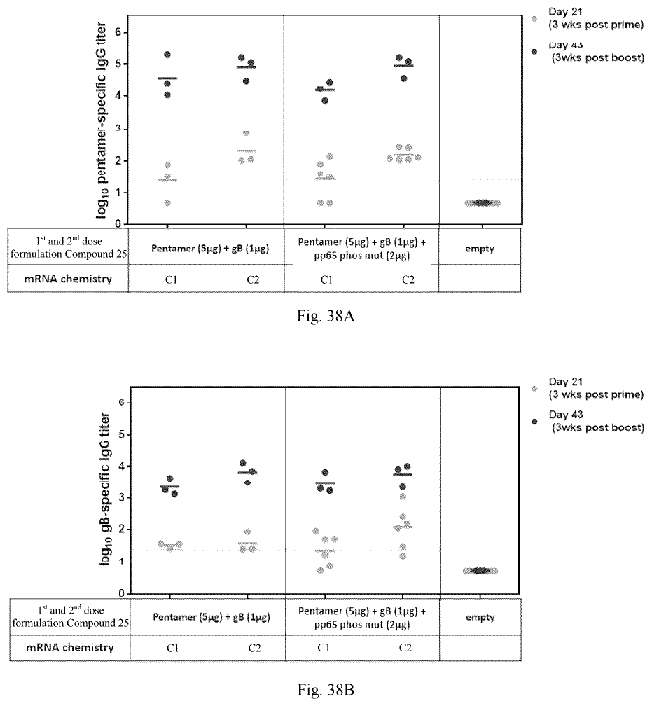

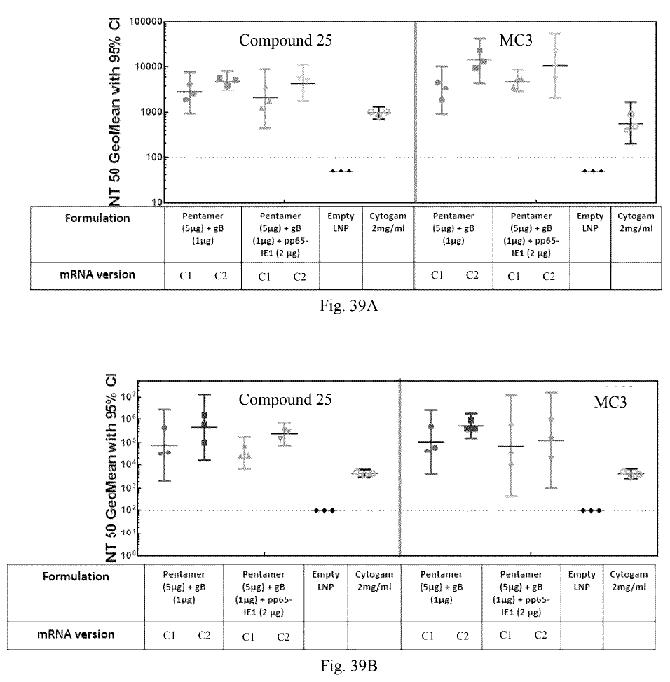

The sera were analyzed for antibody titers against hCMV pentamer and gB (FIGS. 37A, 37B, 38A, and 38B) and for neutralizing antibody titers against hCMV infection in fibroblast cells (FIG. 39A) or epithelial cells (FIG. 39B). The hCMV mRNA vaccine constructs with (C2,) or without (C1) N1-methylpseudouridine chemical modification were able to elicit antibodies against the pentamer, or gB. Neutralizing antibodies against hCMV infection were also elicited.

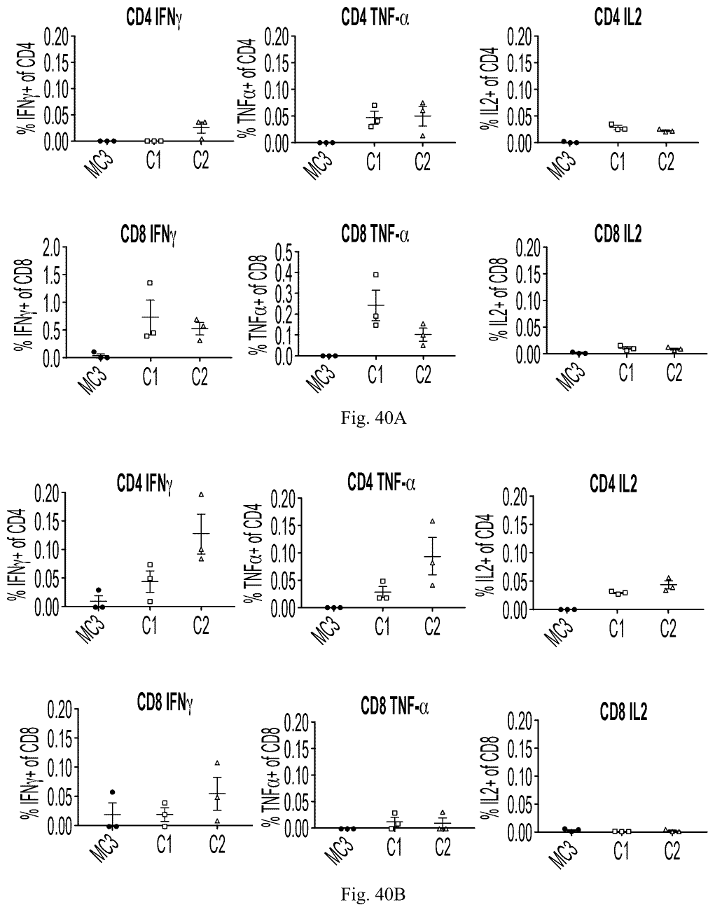

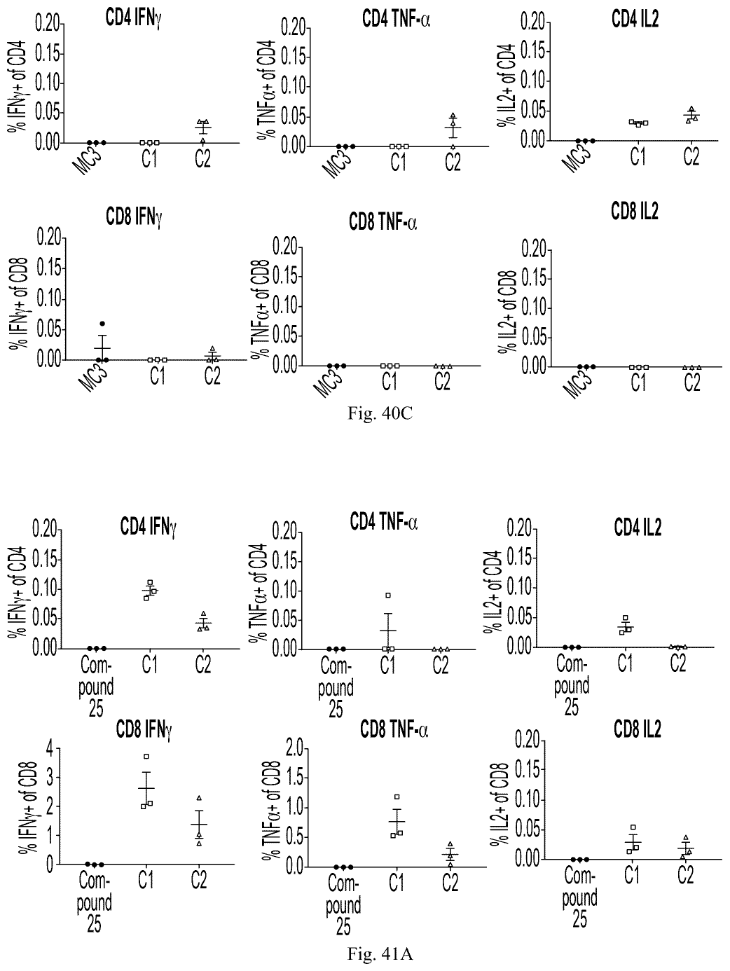

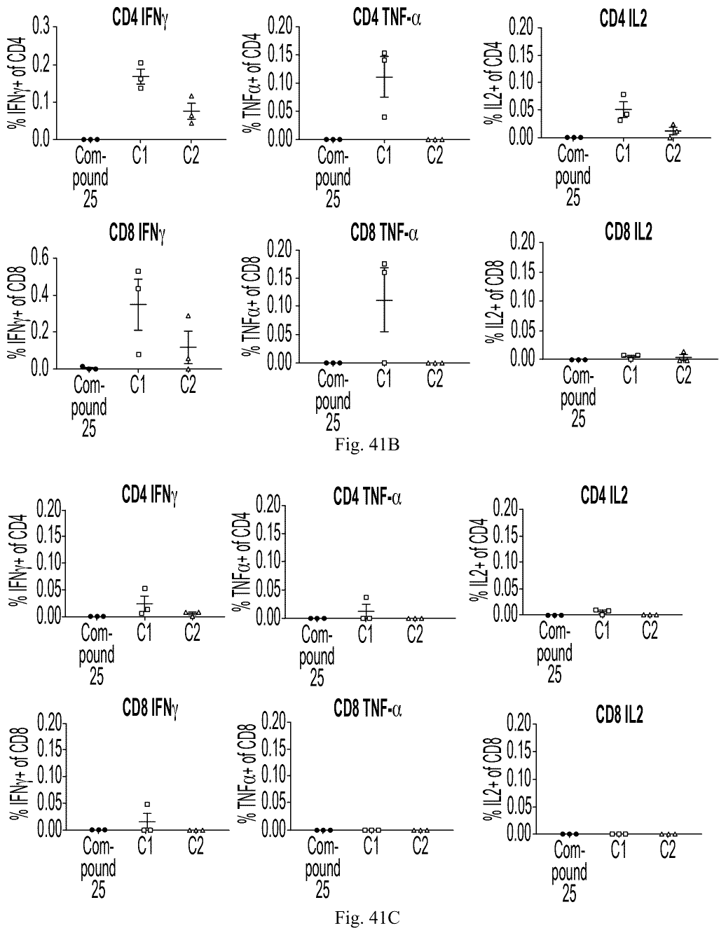

For mice immunized with hCMV mRNA vaccine constructs encoding hCMV pentamer, gB, and pp65mut, T-cell responses (CD4+ and CD8+ T-cell responses as indicated by cytokine secretion) were also evaluated. The results show hCMV mRNA constructs containing the N1-methylpseudouridine (C2) chemical modification formulated in MC3 lipid particles elicited CD4+ and CD8+ T-cell responses against pp65 (FIG. 40B), while the unmodified mRNA constructs formulated in compound 25 lipid particles elicited CD4+ and CD8+ T-cell responses against pp65 (FIG. 41B). Both unmodified hCMV mRNA constructs or mRNA constructs containing the N1-methylpseudouridine (C2) chemical modification formulated in either MC3 or compound 25 lipid particles elicited T-cell responses against the pentamer (FIG. 40A and FIG. 41A).

### Example 36 Maintaining Strong T Cell Response to Pp65 by Sequential Immunization

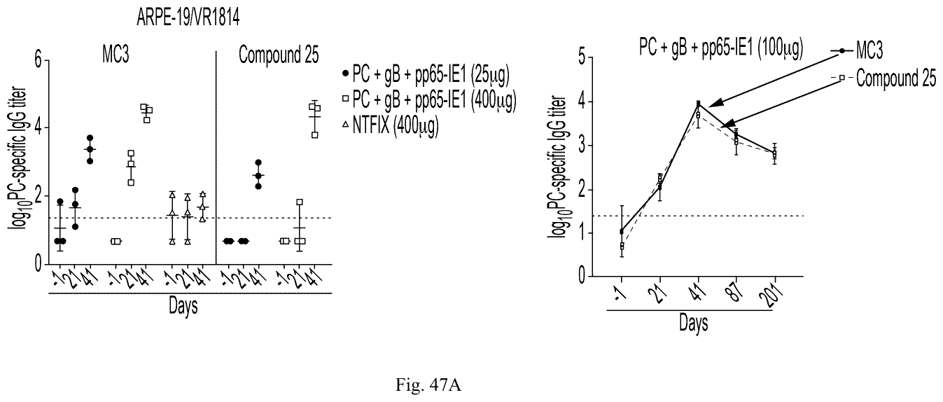

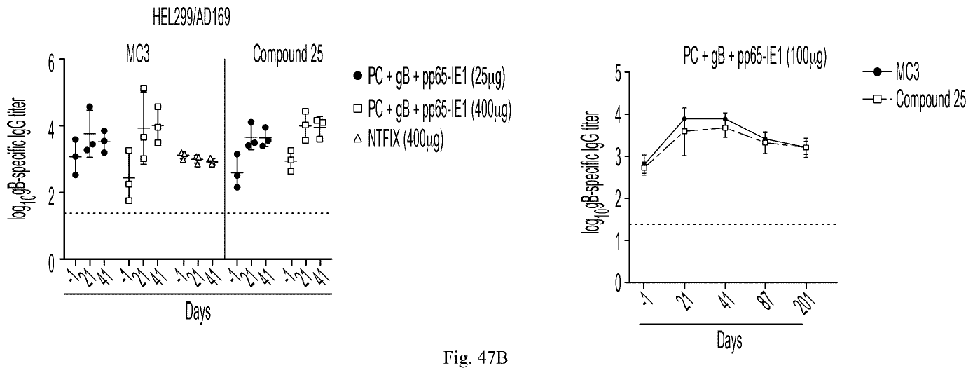

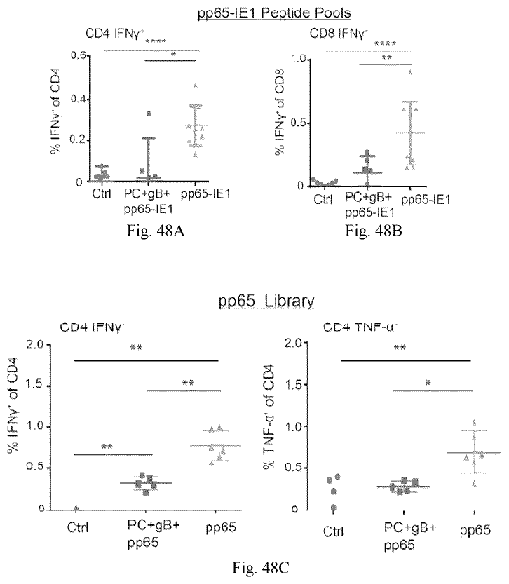

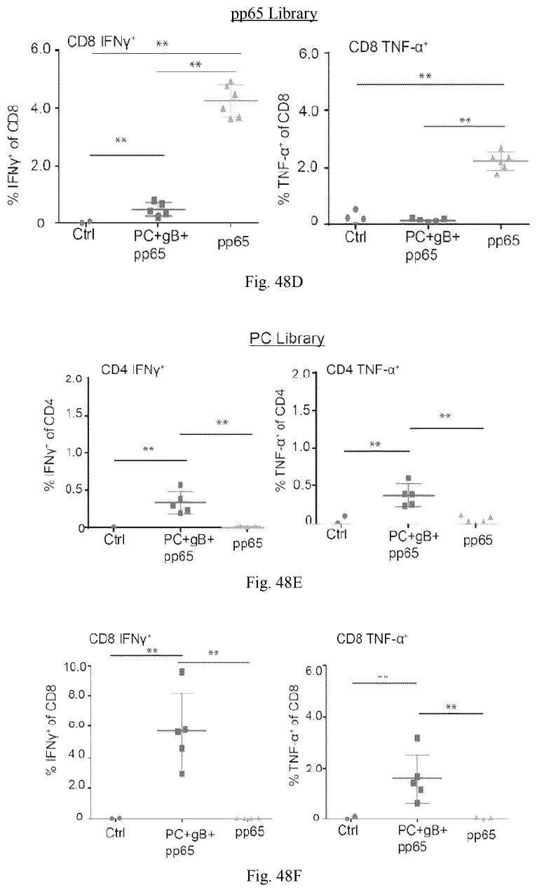

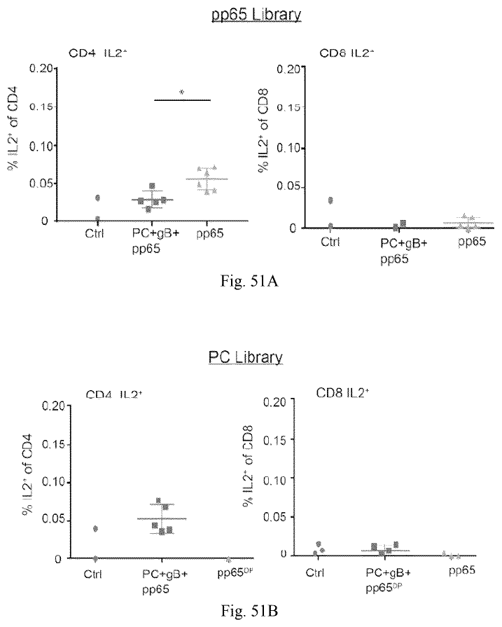

The CMV proteins pp65 and IE1 have emerged as attractive vaccine antigens due to high antigen-specific T cell precursor frequencies in CMV-seropositive individuals. T cell responses to pp65-IE1 in mice were evaluated using intracellular staining assay (ICS). Splenocytes were stimulated with peptide pools comprising select immunodominant peptides for pp65 and IE1 (Reap et al., 2007), and IFN-γ-producing T cells were measured by flow cytometry. In mice that were immunized only with pp65-IE1, IFNγ production was detected in both CD4 and CD8 T cells (FIGS. 48A and 48B). A reduction in pp65-IE1-specific T cell responses was observed when it was coformulated with PC and gB mRNAs (FIGS. 48A and 48B). An mRNA construct encoding a phosphorylation mutant of pp65 (pp65^{ΔP}) that has been reported to retain immunogenicity but exhibit reduced biologic activity was synthesized (Zaia et al., 2009). T cell responses to pp65^{ΔP} were evaluated and compared to pp65-IE1 immunization harboring the same mutation. Splenocytes were stimulated with overlapping peptide library for pp65 and IFNγ producing CD4 and CD8 T cells evaluated by flow cytometry. In this embodiment, the overall T cell responses were higher in mice receiving pp65^{ΔP} as compared to pp65V-IE1 (FIGS. 50A and 50B). Therefore, pp65^{ΔP} was used in the vaccine formulations.

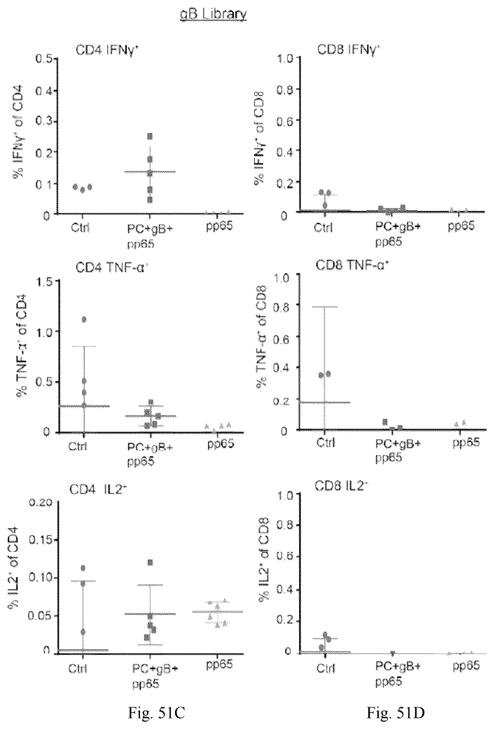

Next, it was evaluated whether T cell responses to pp65 were repressed in the presence of other CMV antigens. Mice were immunized either with LNP encapsulating pp65 alone or pp65+PC+gB. Splenocytes were stimulated with overlapping peptide libraries for pp65, and antigen-specific polyfunctional T cell responses were analyzed by ICS. Robust T cell responses were seen in mice immunized with pp65 alone; the majority of the T cells produced IFNγ and TNF-α (FIGS. 48C and 48D) and, to a lesser extent, IL-2 (FIG. 51A). However, in this embodiment, the pp65-specific T cell responses were reduced in the presence of other CMV antigens (FIGS. 48C and 48D).

To determine whether pp65-specific T cell responses were repressed by other dominant antigens present in the multivalent vaccine, antigen-specific T cell responses to PC and gB were evaluated by ICS. Strong polyfunctional T cell responses to PC (FIGS. 48E and 48D) and little to modest gB-specific T cell responses (FIGS. 51C and 51D) were observed. The majority of these PC-specific T cells secreted IFNγ and TNFα (FIGS. 5E-5F) and, to a minor extent, IL-2 (FIG. 51B). These results suggest that the inhibition of T cell responses to pp65 stems from epitope competition due to dominating epitopes present in PC.

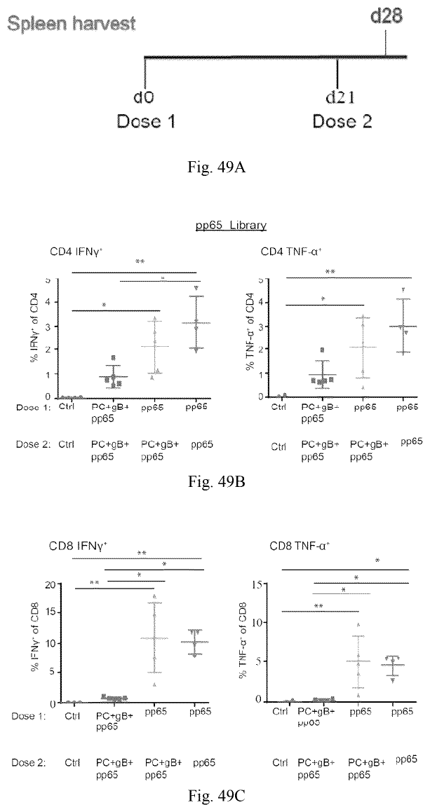

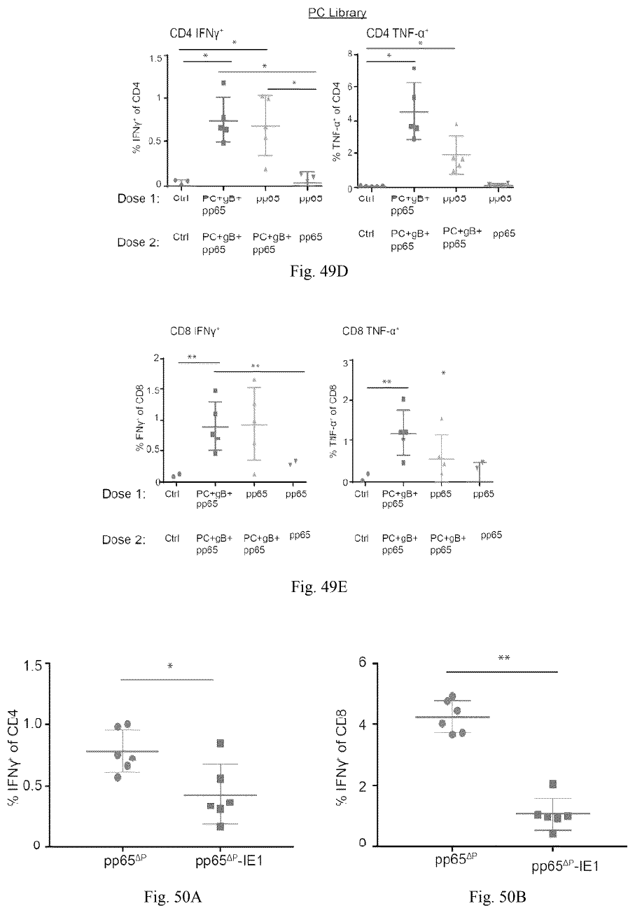

Epitope competition was found to be resolved by a heterologous prime/boost regimen of a dose of LNP (pp65) followed by a dose of LNP (PC+gB+pp65). Control mice were immunized with a homologous prime/boost regimen of LNP (PC+gB+pp65) or a homologous prime/boost regimen of LNP (pp65) according to the dosing regimen shown in FIG. 49A. In mice that received a first dose of pp65 alone followed by all three antigens, T cell responses were restored to levels comparable with those of mice receiving two doses of pp65 alone (FIGS. 49B and 49C). As expected, mice that received two doses of LNP (PC+gB+pp65) showed robust PC-specific T cell responses and negligible responses to pp65 (FIGS. 49D and 49E). These results demonstrate that epitope competition can be resolved by a heterologous and sequential prime/boost vaccine regimen.

### Example 37 Materials and Methods for Examples 22-28, 31-33, 35, and 38

### Amimal Studies

Eight to ten week old female BALB/c mice (Charles River Laboratories International, Inc.; Wilmington, MA) were immunized by intramuscular injection with 50 μl of the indicated LNP/mRNA formulations or empty LNP. All mouse studies were approved by the Animal Care and Use Committee at Moderna Therapeutics, Cambridge, MA.

### Non-Human Primate Experiments

NHP studies were carried out at Southern Research Institute, Frederick, MD. Cynos 2-5 years old weighing 3 kg-6 kg were immunized twice with varying doses of two different LNP formulations (MC3 and Compound 25) containing the mRNA constructs encoding CMV pentamer, gB, and pp65-IE1 antigens. Injections were given intramuscularly in a volume of 0.5 ml. All monkeys were screened for cyCMV and included in the study based on neutralization titers to CMV. The animal protocol was approved by the Institutional Animal Care and Use Committee (IACUC) at Southern Research Institute.

### Cells and Virus

HEK293, HeLa, HEL 299, and ARPE-19 cells were obtained from American Type Culture Collection (ATCC). All cells were cultured in Dulbecco's Modified Eagle's Medium (DMEM) with 10% fetal bovine serum (FBS) and 1% penicillin and streptomycin. HUVEC cells (ATCC) were cultured in endothelial cell growth medium. CMV strain AD169 (ATCC) was propagated on MRC-5 cells and VR1814 (G. Gerna, Fondazione IRCCS Policlinico San Matteo; Pavia, Italy) on HUVEC cells. Clarified supernatants were collected 10 days after 90% of cells showed cytopathic effect. Viral stocks were generated by adding FBS to a final concentration of 20%.

### Western Blot and Immunoprecipitations

HEK293 cells were transiently transfected with mRNA encoding gH, gL, UL128, UL130, UL131A, or gB using Trans IT®-mRNA Transfection Kit (Minus Bio LLC) per the manufacturer's recommendations. At 24 hr post-transfection, cells were lysed in RIPA buffer (Boston BioProducts) supplemented with complete mini-EDTA free protease inhibitor cocktail tablets (ThermoFisher Scientific). Precleared lysates were resolved on Novex 4%-12% Bis-Tris gels (Invitrogen) and blotted with rabbit polyclonal antibodies for gH, gL, UL128, UL130, or UL131A. (D. Johnson, OHSU; Portland, OR) and mouse anti-β actin (Cell Signaling Technology). Alexa Fluor 488 goat antirabbit IgG or Alexa Fluor 680 goat antimouse IgG (ThermoFisher Scientific) were used as secondary antibodies. All images were captured on a ChemiDoc MP Imaging System (Bio-Rad Laboratories). For immunoprecipitations, lysates were first precleared with Protein G agarose beads (ThermoFisher Scientific), and gB was immunoprecipitated using an anti-gB monoclonal antibody (clone CH28, Santa Cruz Biotechnology). Immunoprecipitates were resolved on Novex 4%-12% Bis-Tris gels (Invitrogen) and probed with mouse anti-gB antibody followed by incubation with HRP-conjugated rat antimouse IgG that recognizes native mouse IgG (Mouse TrueBlot® Western Blot Kit, Rockland Inc). Immunoblots were developed using TrueBlot substrate (Rockland Inc.) and visualized on a ChemiDoc MP Imaging System (Bio-Rad Laboratories, Inc.).

### Flow Cytometry

HeLa cells were transiently transfected with mRNA for the various subunits of CMV PC (gH/gL/UL128/UL130/UL131A) or combinations lacking one of the subunits or gB. After 24 hr, the cells were harvested and resuspended in FACS buffer (1× PBS, 3% FBS, 0.05% sodium azide). To detect surface PC expression or components of PC, the cells were stained with human monoclonal antibodies 8I21 (PC), 3G16 (gH), 15D8var1 (UL128), and 7113 (UL128/UL130/UL131A) (Macagno et al., 2010). All the above human monoclonal antibodies were custom synthesized by ThermoFisher from Expi293 cells that were transfected with expression plasmids encoding codon-optimized sequences for the respective heavy and light chain antibody. Surface gB was detected by mouse monoclonal anti-gB (Santa Cruz Biotechnology, Inc.). To detect intracellular gB, cells were permeabilized with 1× Cytofix/Cytoperm™ (BD Biosciences) and stained with mouse monoclonal anti-gB (Santa Cruz Biotechnology, Inc.). Alexaflour 647 goat antihuman IgG (SouthernBiotech) or Alexafluor 647 goat antimouse IgG (SouthernBiotech) were used as secondary antibodies. Cells were acquired on a BD LSRII Fortessa instrument (BD Biosciences) and analyzed by FlowJo software v10 (Tree Star, Inc.),

### Intracellular Cytokine Staining

Overlapping peptide libraries for gH, gL, UL128, UL130, UL131A (15 mer overlapping by 5 amino acids) and gB (15 mer overlapping by 11 amino acids) were synthesized by Genscript (Piscataway, NJ). A peptide library for PC was generated by pre-mixing the peptide pools for the five different components of the complex. The pp65 peptide library (15 mer overlapping by 11 amino acids) was from JPT Inc. Splenocytes were stimulated with peptides pools for PC, gB, and pp65 at 10 μg/ml for 5 hr at 37° C. in the presence of BD GolgiStop™ and GolgiPlug™ (BD Biosciences). Unstimulated or PMA/lonomycin (Cell Stimulation Cocktail, eBioscience) were used as negative and positive controls, respectively. Following stimulation, cells were surface stained in FACS buffer in the presence of FcR blocking antibody 2.4G2 and eFluor™ 506 (eBioscience) as viability dye. Antibody clones used for surface staining were: anti-CD4 (GK1.5), anti-CD8 (53.6.7), anti-CD44 (IM7), anti-CD62L (MEL14), and anti-TCRβ (H57-59). Intracellular staining was carried out with BD Cytofix/Cytoperm and BD Perm/Wash™ buffers (BD Biosciences). Antibody clones used for intracellular staining were: anti-IFNγ (XMG1.2), anti-1L2 (JES6-5H4) and anti-TNFα (MP6-XT22). Samples were acquired on BD LSRII Fortessa (BD Biosciences) and analyzed by FlowJo software (TreeStar, Inc.). Cytokine secreting T cells were plotted after background subtraction.

Generation of Modified CMV mRNA Vaccine Constructs and Formulations

Generation of mRNA encoding CMV antigens gH, gL, UL128, UL130, UL131A. and gB from strain Merlin was done by in vitro transcription using T7 polymerase from a linear DNA template that included 5′ and 3′ untranslated regions (UTRs) and a poly (A) tail as previously described (Richner et al., 2017b). mRNA encoding a phosphorylation mutant of pp65 (pp65^{ΔP}) was generated by deleting a.a 435-438 (RKRK). A pp65/IE1 fusion mRNA was constructed by assembling in tandem the sequences of pp65 gene lacking the stop codon with IE1 gene without the start codon to generate an in-frame fusion gene. S-adenosylmethionine was added to the methylated capped RNA (cap1) for increased mRNA translation efficiency. Similarly, a pp65^{ΔP}-IE1 mRNA construct lacking a.a 435-438 of pp665 was also generated. LNPs were formulated as previously described (Chen et al., 2016). Briefly, lipids were dissolved in ethanol at molar ratios of 50:10:38.5:1.5 (ionizable lipid:DSPC:cholesterol:PEG lipid). Two different LNPs having different ionizable lipids, referred to as MC3 and Compound 25, respectively, were developed. mRNA was combined with the lipid mixture, dialyzed and concentrated as previously described (Richner et al., 2017b). Empty LNPs lacking mRNA were also generated as controls. All formulations had particle sizes ranging from 80 nm to 100 nm, with greater than 90% encapsulation and <1 EU/ml of endotoxin.

### ELISA

Overnight, 96-well microtiter plates were coated with 1 μg/ml of PC (Native Antigen Company) or gB (Sino Biological) protein. Serial dilutions of serum were added and bound antibody detected with HRP-conjugated goat antimouse IgG (Southern Biotech), followed by incubation with TMB substrate (KPL). The absorbance was measured at OD (450 nm). Titers were determined using a four parameter logistic curve fit in GraphPad Prism (GraphPad Software. Inc.) and defined as the reciprocal serum dilution at approximately OD (450 nm)=0.6 (normalized to a standard on each plate).

### Neutralization Assays

Serum samples were heat inactivated at 56° C. for 30 min and diluted 1:50 in complete medium. Cytogam was diluted to 10 mg/ml. Thereafter, samples were serially diluted in 2-fold steps and mixed with an equal volume of VR 1814 or AD169 virus in serum-free media supplemented with 10% guinea pig complement (Cedarlane Laboratories Ltd) and incubated for 4 hr at 37° C., 5% CO2. The virus/serum mixture was then added to ARPE-19 or MRC-5 cells in 96-well tissue culture plates and incubated for 17-20 hr at 37° C., 5% CO2. Cells were fixed with 200 proof ethanol, blocked with superblock (Sigma-Aldrich), washed with PBS/0.05% Tween-20, and stained with mouse monoclonal antibody to CMV IE1 (Millipore), followed by Peroxidase AffiniPure Goat Anti-Mouse IgG (Jackson ImmunoResearch Laboratories) and developed with HistoMark® TrueBlue™ Peroxidase Substrate (SeraCare). CMV IE1-positive cells were counted using the CTL ImmunoSpot® Analyzer (Cellular Technology Limited). Neutralization titers (NT50) were determined using a four parameter logistic curve fit in GraphPad Prism (GraphPad Software, Inc.) and were defined as the reciprocal of the serum dilution resulting in 50% reduction in infected-cell count. In all experiments, the titers of Cytogam (CSL Behring) are shown for an approximate maximum concentration (2 mg/ml) in human sera after dosing, which was calculated based on an average body weight of 70 kg.

### Statistical Analysis

Data were analyzed with Prism 7 (GraphPad Software) using the Kruskal-Wallis test and Dunn's multiple comparison test or by two-tailed Mann-Whitney U test. A p value of <0.05 indicated statistically significant differences.

TABLE 12
Table of Materials

| | | |
|---|---|---|
| REAGENT or RESOURCE | SOURCE | IDENTIFIER |

Antibodies

| | | |
|---|---|---|
| Rabbit anti-gH | Johnson Laboratory | N/A |
| Rabbit anti-gL | Johnson Laboratory | N/A |
| Rabbit anti-UL128 | Johnson Laboratory | N/A |
| Rabbit anti-UL130 | Johnson Laboratory | N/A |
| Rabbit anti-UL131A | Johnson Laboratory | N/A |
| 8I21 | Thermofisher | N/A |
| 3G16 | Thermofisher | N/A |
| 15D8Var1 | Thermofisher | N/A |
| 7I13 | Thermofisher | N/A |
| Anti-mouse CD16/32 (2.4G2) | BD Biosciences | Cat# 553141 |
| Anti-mouse CD4 (GK1.5) | Biolegend | Cat# 100437 |
| Anti-mouse CD8 (53.6.7) | Biolegend | Cat# 100705 |
| Anti-mouse CD44 (IM7) | Biolegend | Cat# 103030 |
| Anti-mouse CD62L (MEL14) | Biolegend | Cat# 104428 |
| Anti-mouse TCRβ (H57-59) | Biolegend | Cat# 109224 |
| Anti-mouse IFNα (XMG 1.2) | Thermofisher | Cat# 17-7311-82 |
| Anti-mouse IL2 (JES6-5H4) | Biolegend | Cat# 503829 |
| Anti-mouse TNFα (MP6-XT22) | Thermofisher | Cat# 12-7321-41 |
| Mouse anti-β actin | Cell Signaling | Cat# 3700S |
| | Technology | |
| Alexa Fluor 488 goat anti-rabbit IgG | Thermofisher | Cat# A21109 |
| Alexa Fluor 680 goat anti-mouse IgG | Thermofisher | Cat# A21057 |
| Alexa Fluor 647 goat anti-human IgG | Southern Biotech | Cat# 2016-31 |
| Alexa Fluor 647 goat anti-mouse IgG | Southern Biotech | Cat# 1031-31 |
| HRP-conjugated rat anti-mouse IgG | Rockland Inc | Cat# 18-8817-31 |
| HRP-conjugated goat anti-mouse IgG | Southern Biotech | Cat# 1030-05 |
| Peroxidase AffiniPure Goat Anti-mouse IgG | Jackson | 115-035-166 |
| | ImmunoResearch | |
| Mouse anti-gB (clone CH28) | Santa Cruz | sc-69742 |
| | Biotechnology | |
| Mouse anti-CMV IE1 | Millipore | MAB810 |

Bacterial and Virus Strains

| | | |
|---|---|---|
| AD169 | ATCC | VR-538 |
| VR1814 | Gerna lab | N/A |

Chemicals, Peptides, and Recombinant Proteins

| | | |
|---|---|---|
| RIPA Lysis Buffer | Boston Bio Products | BP-115 |
| Complete mini-EDTA free protease inhibitor tablets | Thermofisher | Cat# 88266 |
| Cytofix/Cytoperm | BD Biosciences | Cat# 554714 |
| GolgiStop | BD Biosciences | Cat# 554724 |
| GolgiPlug | BD Biosciences | Cat# 555029 |
| eFluor ™ 506 Fixable Viability Dye | Thermofisher | Cat# 65-0866-14 |
| PC peptide library | Genscript | N/A |

TABLE 13
Additional hCMV Vaccine Constructs

| | | | Tested |
|---|---|---|---|
| | | | Chemistry |
| | | | mRNA |
| | | | Chem- |
| | | | istry/ |
| Antigen | | | Cap/ |
| Name | Protein Sequence | Nucleotide Sequence (Open Reading Frames) | Tail |
| CMV IE1 | MESSAKRKMDPD | ATGGAGTCCTCTGCCAAGAGAAAGATGGACCCTGATA | C2/ |
| (UL122) | NPDEGPSSKVPRP | ATCCTGACGAGGGCCCTTCCTCCAAGGTGCCACGGCCC | CAP1/ |
| | ETPVTKATTFLQT | GAGACACCCGTGACCAAGGCCACGACGTTCCTGCAGA | T100 |
| | MLRKEVNSQLSLG | CTATGTTAAGGAAGGAGGTTAACAGTCAGCTGAGCCT | |
| | DPLFPELAEESLKT | GGGAGACCCGCTGTTCCCAGAATTGGCCGAAGAATCC | |
| | FEQVTEDCNENPE | CTCAAGACCTTTGAACAAGTGACCGAGGATTGCAACG | |
| | KDVLTELVKQIKV | AGAACCCCGAGAAAGATGTCCTGACAGAACTCGTCAA | |
| | RVDMVRHRIKEH | ACAGATTAAGGTTCGAGTGGACATGGTGCGGCATAGA | |
| | MLKKYTQTEEKFT | ATCAAGGAGCACATGCTGAAGAAATATACCCAGACGG | |
| | GAFNMMGGCLQN | AAGAGAAATTCACTGGCGCCTTTAATATGATGGGAGG | |
| | ALDILDKVHEPFE | ATGTTTGCAGAATGCCTTAGATATCTTAGATAAGGTTC | |
| | DMKCIGLTMQSM | ATGAGCCTTTCGAGGACATGAAGTGTATTGGGCTAACT | |
| | YENYIVPEDKREM | ATGCAGAGCATGTATGAGAACTACATTGTACCTGAGG | |
| | WMACIKELHDVS | ATAAGCGGGAGATGTGGATGGCTTGTATTAAGGAGCT | |
| | KGAANKLGGALQ | GCATGATGTGAGCAAGGGCGCCGCTAACAAGTTGGGC | |
| | AKARAKKDELRR | GGTGCACTGCAGGCTAAGGCCCGTGCTAAGAAGGATG | |
| | KMMYMCYRNIEF | AACTTAGGAGAAAGATGATGTATATGTGCTACAGGAA | |
| | FTKNSAFPKTTNG | TATAGAGTTCTTTACCAAGAACTCAGCCTTCCCTAAGA | |
| | CSQAMAALQNLP | CCACCAATGGCTGCAGTCAGGCCATGGCGGCATTGCA | |
| | QCSPDEIMSYAQK | GAACTTGCCTCAGTGCTCTCCTGATGAGATTATGTCTT | |
| | IFKILDEERDKVLT | ATGCCCAGAAGATCTTTAAGATTTTGGATGAGGAGAG | |
| | HIDHIFMDILTTCV | AGACAAGGTGCTCACGCACATTGATCACATATTTATGG | |
| | ETMCNEYKVTSD | ATATCCTCACTACATGTGTGGAAACAATGTGTAATGAG | |
| | ACMMTMYGGISL | TACAAGGTCACTAGTGACGCTTGTATGATGACCATGTA | |
| | LSEFCRVLCCYVL | CGGCGGCATCTCTCTCTTAAGTGAGTTCTGTCGGGTGC | |
| | EETSVMLAKRPLI | TGTGCTGCTATGTCTTAGAGGAGACTAGTGTGATGCTG | |
| | TKPEVISVMKRRIE | GCCAAGCGGCCTCTGATAACCAAGCCTGAGGTTATCA | |
| | EICMKVFAQYILG | GTGTAATGAAGCGCCGCATTGAGGAGATCTGCATGAA | |
| | ADPLRVCSPSVDD | GGTCTTTGCCCAGTACATTCTGGGAGCCGATCCTTTGA | |
| | LRAIAEESDEEEAI | GAGTCTGCTCTCCTAGTGTGGATGACCTACGGGCCATC | |
| | VAYTLATAGASSS | GCCGAGGAGTCAGATGAGGAAGAGGCTATTGTAGCCT | |
| | DSLVSPPESPVPAT | ACACTTTGGCCACCGCTGGTGCCAGCTCCTCTGATTCT | |
| | IPLSSVIVAENSDQ | CTGGTGTCACCTCCAGAGTCCCCTGTACCCGCGACTAT | |
| | EESEQSDEEQEEG | CCCTCTGTCCTCAGTAATTGTGGCTGAGAACAGTGATC | |
| | AQEEREDTVSVKS | AGGAAGAAAGTGAACAGAGTGATGAGGAACAGGAGG | |
| | EPVSEIEEVASEEE | AGGGTGCTCAGGAGGAGCGGGAGGACACTGTGTCTGT | |
| | EDGAEEPTASGGK | CAAGTCTGAGCCAGTGTCTGAGATAGAGGAAGTTGCC | |
| | STHPMVTRSKADQ | TCAGAGGAAGAGGAGGATGGTGCTGAGGAACCCACCG | |
| | (SEQ ID NO: 80) | CCTCTGGAGGCAAGAGCACCCACCCTATGGTGACTAG | |
| | | AAGCAAGGCTGACCAG (SEQ ID NO: 84) | |
| CMV IE2 | MESSAKRKMDPD | ATGGAGTCCTCTGCCAAGAGAAAGATGGACCCTGATA | C2/ |
| | NPDEGPSSKVPRP | ATCCTGACGAGGGCCCTTCCTCCAAGGTGCCACGGCCC | CAP1/ |
| | ETPVTKATTFLQT | GAGACACCCGTGACCAAGGCCACGACGTTCCTGCAGA | T100 |
| | MLRKEVNSQLSLG | CTATGTTAAGGAAGGAGGTTAACAGTCAGCTGAGCCT | |
| | DPLFPELAEESLKT | GGGAGACCCGCTGTTCCCAGAATTGGCCGAAGAATCC | |
| | FEQVTEDCNENPE | CTCAAGACCTTTGAACAAGTGACCGAGGATTGCAACG | |
| | KDVLTELGDILAQ | AGAACCCCGAGAAAGATGTCCTGACAGAACTCGGTGA | |
| | AVNHAGIDSSSTG | CATCCTCGCCCAGGCTGTCAATCATGCCGGTATCGATT | |
| | PTLTTHSCSVSSAP | CCAGTAGCACCGGCCCCACGCTGACAACCCACTCTTGC | |
| | LNKPTPTSVAVTN | AGCGTTAGCAGCGCCCCTCTTAACAAGCCGACGCCCA | |
| | TPLPGASATPELSP | CCAGCGTCGCGGTTACTAACACTCCTCTCCCCGGGGCA | |
| | RKKPRKTTRPFKV | TCCGCTACTCCCGAGCTCAGCCCGCGTAAGAAACCGC | |
| | IIKPPVPPAPIMLPL | GCAAGACCACGCGTCCTTTCAAGGTGATTATTAAACCG | |
| | IKQEDIKPEPDFTI | CCCGTGCCTCCCGCGCCTATCATGCTGCCCCTCATCAA | |
| | QYRNKIIDTAGCIV | ACAGGAAGACATCAAGCCCGAGCCCGACTTTACCATC | |
| | ISDSEEEQGEEVET | CAGTACCGCAACAAGATTATCGATACCGCCGGCTGTA | |
| | RGATASSPSTGSG | TCGTGATCTCTGATAGCGAGGAAGAACAGGGTGAAGA | |
| | TPRVTSPTHPLSQ | AGTCGAGACCCGCGGTGCTACCGCGTCTTCCCCTTCCA | |
| | MNHPPLPDPLGRP | CCGGCAGCGGCACGCCGCGAGTGACCTCTCCCACGCA | |
| | DEDSSSSSSSSCSS | CCCGCTCTCCCAGATGAACCACCCTCCTCTTCCCGATC | |
| | ASDSESESEEMKC | CTTTGGGCCGGCCCGATGAAGATAGTTCCTCTTCGTCT | |
| | SSGGGASVTSSHH | TCCTCCTCCTGCAGTTCGGCTTCGGACAGCGAGAGTGA | |
| | GRGGFGGAASSSL | GTCCGAGGAGATGAAATGCAGCAGTGGCGGAGGAGC | |
| | LSCGHQSSGGAST | ATCCGTGACCTCGAGCCACCATGGGCGCGGCGGTTTTG | |
| | GPRKKKSKRISEL | GTGGCGCGGCCTCCTCCTCTCTGCTGAGCTGCGGACAT | |
| | DNEKVRNIMKDK | CAGAGCAGCGGCGGGGCGAGCACCGGACCTCGCAAG | |
| | NTPFCTPNVQTRR | AAGAAGAGCAAACGCATCTCCGAGTTGGACAACGAGA | |
| | GRVKIDEVSRMFR | AGGTGCGCAATATCATGAAAGATAAGAACACGCCCTT | |
| | HTNRSLEYKNLPF | CTGCACACCCAACGTGCAGACTCGGCGGGGTCGCGTC | |
| | MIPSMHQVLEEAI | AAGATTGACGAGGTGAGCCGCATGTTCCGTCACACCA | |
| | KVCKTMQVNNKG | ATCGTTCTCTTGAGTACAAGAATCTGCCATTCATGATC | |
| | IQIIYTRNHEVKNE | CCTAGTATGCACCAAGTGTTAGAAGAGGCCATCAAAG | |
| | VDQVRCRLGSMC | TTTGCAAGACCATGCAGGTGAACAACAAGGGCATTCA | |
| | NLALSTPFLMEHT | GATCATCTACACCCGCAATCATGAAGTGAAGAATGAG | |
| | MPVTHPPDVAQRT | GTGGATCAGGTACGGTGTCGCCTGGGTAGCATGTGCA | |
| | ADACNDGVKAV | ACCTGGCCCTCTCCACTCCCTTCCTCATGGAGCACACT | |
| | WNLKELHTHQLC | ATGCCTGTGACACACCCTCCTGATGTGGCGCAGCGCAC | |
| | PRSSDYRNMIIHA | GGCCGATGCTTGTAACGACGGTGTCAAGGCCGTGTGG | |
| | ATPVDLLGALNLC | AACCTCAAAGAACTGCACACCCACCAATTGTGCCCGC | |
| | LPLMQKFPKQVM | GCTCTTCTGATTACCGCAACATGATTATCCACGCTGCC | |
| | VRIFSTNQGGFML | ACGCCCGTGGACCTGTTGGGCGCTCTCAACCTGTGCCT | |
| | PIYETAAKAYAVG | GCCCCTGATGCAGAAGTTTCCCAAACAGGTCATGGTG | |
| | QFEKPTETPPEDLD | CGCATCTTCTCCACCAACCAGGGTGGGTTCATGCTGCC | |
| | TLSLAIEAAIQDLR | TATCTACGAGACGGCCGCGAAGGCCTACGCCGTGGGG | |
| | NKSQ (SEQ ID NO: | CAGTTTGAGAAGCCCACCGAGACCCCTCCCGAAGACC | |
| | 81) | TGGACACCCTGAGCCTGGCCATCGAGGCAGCCATCCA | |
| | | GGACCTGAGGAACAAATCTCAG (SEQ ID NO: 85) | |
| hCMV_gB | MESRIWCLVVCV | ATGGAATCCAGGATCTGGTGCCTGGTAGTCTGCGTTAA | C2/ |
| | NLCIVCLGAAVSS | CTTGTGTATCGTCTGTCTGGGTGCTGCGGTTTCCTCATC | Cap1/ |
| | SSTRGTSATHSHH | TTCTACTCGTGGAACTTCTGCTACTCACAGTCACCATT | Tailless |
| | SSHTTSAAHSRSG | CCTCTCATACGACGTCTGCTGCTCACTCTCGATCCGGT | C2/no |
| | SVSQRVTSSQTVS | TCAGTCTCTCAACGCGTAACTTCTTCCCAAACGGTCAG | cap/T100 |
| | HGVNETIYNTTLK | CCATGGTGTTAACGAGACCATCTACAACACTACCCTCA | C1/cap1/ |
| | YGDVVGVNTTKY | AGTACGGAGATGTGGTGGGGGTCAATACCACCAAGTA | T100 |
| | PYRVCSMAQGTD | CCCCTATCGCGTGTGTTCTATGGCCCAGGGTACGGATC | |
| | LIRFERNIVCTSMK | TTATTCGCTTTGAACGTAATATCGTCTGCACCTCGATG | |
| | PINEDLDEGIMVV | AAGCCCATCAATGAAGACCTGGACGAGGGCATCATGG | |
| | YKRNTVAHTFKVR | TGGTCTACAAACGCAACATCGTCGCGCACACCTTTAAG | |
| | VYQKVLTFRRSYA | GTACGAGTCTACCAGAAGGTTTTGACGTTTCGTCGTAG | |
| | YIHTTYLLGSNTE | CTACGCTTACATCCACACCACTTATCTGCTGGGCAGCA | |
| | YVAPPMWEIHHIN | ACACGGAATACGTGGCGCCTCCTATGTGGGAGATTCA | |
| | SHSQCYSSYSRVIA | TCATATCAACAGCCACAGTCAGTGCTACAGTTCCTACA | |
| | GTVFVAYHRDSYE | GCCGCGTTATAGCAGGCACGGTTTTCGTGGCTTATCAT | |
| | NKTMQLMPDDYS | AGGGACAGCTATGAAAACAAAACCATGCAATTAATGC | |
| | NTHSTRYVTVKD | CCGACGATTATTCCAACACCCACAGTACCCGTTACGTG | |
| | QWHSRGSTWLYR | ACGGTCAAGGATCAATGGCACAGCCGCGGCAGCACCT | |
| | ETCNLNCMVTITT | GGCTCTATCGTGAGACCTGTAATCTGAATTGTATGGTG | |
| | ARSKYPYHFFATS | ACCATCACTACTGCGCGCTCCAAATATCCTTATCATTT | |
| | TGDVVDISPFYNG | TTTCGCCACTTCCACGGGTGACGTGGTTGACATTTCTC | |
| | TNRNASYFGENAD | CTTTCTACAACGGAACCAATCGCAATGCCAGCTACTTT | |
| | KFFIFPNYTIVSDF | GGAGAAAACGCCGACAAGTTTTTCATTTTTCCGAACTA | |
| | GRPNSALETHRLV | CACTATCGTCTCCGACTTTGGAAGACCGAATTCTGCGT | |
| | AFLERADSVISWDI | TAGAGACCCACAGGTTGGTGGCTTTTCTTGAACGTGCG | |
| | QDEKNVTCQLTF | GACTCGGTGATCTCCTGGGATATACAGGACGAAAAGA | |
| | WEASERTIRSEAE | ATGTCACTTGTCAACTCACTTTCTGGGAAGCCTCGGAA | |
| | DSYHFSSAKMTAT | CGCACCATTCGTTCCGAAGCCGAGGACTCGTATCACTT | |
| | FLSKKQEVNMSDS | TTCTTCTGCCAAAATGACCGCCACTTTCTTATCTAAGA | |
| | ALDCVRDEAINKL | AGCAAGAGGTGAACATGTCCGACTCTGCGCTGGACTG | |
| | QQIFNTSYNQTYE | CGTACGTGATGAGGCTATAAATAAGTTACAGCAGATT | |
| | KYGNVSVFETTGG | TTCAATACTTCATACAATCAAACATATGAAAAATATGG | |
| | LVVPWQGIKQKSL | AAACGTGTCCGTCTTTGAAACCACTGGTGGTTTGGTAG | |
| | VELERLANRSSLN | TGTTCTGGCAAGGTATCAAGCAAAAATCTCTGGTGGA | |
| | LTHNRTKRSTDGN | ACTCGAACGTTTGGCCAACCGCTCCAGTCTGAATCTTA | |
| | NATHLSNMESVH | CTCATAATAGAACCAAAAGAAGTACAGATGGCAACAA | |
| | NLVYAQLQFTYDT | TGCAACTCATTTATCCAACATGGAATCGGTGCACAATC | |
| | LRGYINRALAQIA | TGGTCTACGCCCAGCTGCAGTTCACCTATGACACGTTG | |
| | EAWCVDQRRTLE | CGCGGTTACATCAACCGGGCGCTGGCGCAAATCGCAG | |
| | VFKELSKINPSAIL | AAGCCTGGTGTGTGGATCAACGGCGCACCCTAGAGGT | |
| | SAIYNKPIAARFM | CTTCAAGGAACTCAGCAAGATCAACCCGTCAGCCATT | |
| | GDVLGLASCVTIN | CTCTCGGCCATTTACAACAAACCGATTGCCGCGCGTTT | |
| | QTSVKVLRDMNV | CATGGGTGATGTCTTGGGCCTGGCCAGCTGCGTGACCA | |
| | KESPGRCYSRPVVI | TCAACCAAACCAGCGTCAAGGTGCTGCGTGATATGAA | |
| | FNFANSSYVQYGQ | CGTGAAGGAGTCGCCAGGACGCTGCTACTCACGACCC | |
| | LGEDNEILLGNHR | GTGGTCATCTTTAATTTCGCCAACAGCTCGTACGTGCA | |
| | TEECQLPSLKIFIA | GTACGGTCAACTGGGCGAGGACAACGAAATCCTGTTG | |
| | GNSAYEYVDYLF | GGCAACCACCGCACTGAGGAATGTCAGCTTCCCAGCC | |
| | KRMIDLSSISTVDS | TCAAGATCTTCATCGCCGGGAACTCGGCCTACGAGTAC | |
| | MIALDIDPLENTDF | GTGGACTACCTCTTCAAACGCATGATTGACCTCAGCAG | |
| | RVLELYSQKELRS | TATCTCCACCGTCGACAGCATGATCGCCCTGGATATCG | |
| | SNVFDLEEIMREF | ACCCGCTGGAAAATACCGACTTCAGGGTACTGGAACT | |
| | NSYKQRVKYVED | TTACTCGCAGAAAGAGCTGCGTTCCAGCAACGTTTTTG | |
| | KVVDPLPPYLKGL | ACCTCGAAGAGATCATGCGCGAATTCAACTCGTACAA | |
| | DDLMSGLGAAGK | GCAGCGGGTAAAGTACGTGGAGGACAAGGTAGTCGAC | |
| | AVGVAIGAVGGA | CCGCTACCGCCCTACCTCAAGGGTCTGGACGACCTCAT | |
| | VASVVEGVATFLK | GAGCGGCCTGGGCGCCGCGGGAAAGGCCGTTGGCGTA | |
| | NPFGAFTIILVAIA | GCCATTGGGGCCGTGGGTGGCGCGGTGGCCTCCGTGG | |
| | VVIITYLIYTRQRR | TCGAAGGCGTTGCCACCTTCCTCAAAAACCCCTTCGGA | |
| | LCTQPLQNLFPYL | GCGTTCACCATCATCCTCGTGGCCATAGCTGTAGTCAT | |
| | VSADGTTVTSGST | TATCACTTATTTGATCTATACTCGACAGCGGCGTTTGT | |
| | KDTSLQAPPSYEE | GCACGCAGCCGCTGCAGAACCTCTTTCCCTATCTGGTG | |
| | SVYNSGRKGPGPP | TCCGCCGACGGGACCACCGTGACGTCGGGCAGCACCA | |
| | SSDASTAAPPYTN | AAGACACGTCGTTACAGGCTCCGCCTTCCTACGAGGA | |
| | EQAYQMLLALAR | AAGTGTTTATAATTCTGGTCGCAAAGGACCGGGACCA | |
| | LDAEQRAQQNGT | CCGTCGTCTGATGCATCCACGGCGGCTCCGCCTTACAC | |
| | DSLDGRTGTQDK | CAACGAGCAGGCTTACCAGATGCTTCTGGCCCTGGCCC | |
| | GQKPNLLDRLRHR | GTCTGGACGCAGAGCAGCGAGCGCAGCAGAACGGTAC | |
| | KNGYRHLKDSDE | AGATTCTTTGGACGGACGGACTGGCACGCAGGACAAG | |
| | EENV (SEQ ID NO: | GGACAGAAGCCCAACCTACTAGACCGACTGCGACATC | |
| | 69) | GCAAAAACGGCTACCGACACTTGAAAGACTCTGACGA | |
| | | AGAAGAGAACGTC (SEQ ID NO: 86) | |
| hCMV_gH | MRPGLPSYLIILAV | ATGCGGCCAGGCCTCCCCTCCTACCTCATCATCCTCGC | C2/ |
| dimer_v2 | CLFSHLLSSRYGA | CGTCTGTCTCTTCAGCCACCTACTTTCGTCACGATATG | Cap1/ |
| | EAVSEPLDKAFHL | GCGCAGAAGCCGTATCCGAACCGCTGGACAAAGCGTT | Tailless |
| | LLNTYGRPIRFLRE | TCACCTACTGCTCAACACCTACGGGAGACCCATCCGCT | C1/Cap1/ |
| | NTTQCTYNSSLRN | TCCTGCGTGAAAATACCACCCAGTGTACCTACAACAG | T100 |
| | STVVRENAISFNFF | CAGCCTCCGTAACAGCACGGTCGTCAGGGAAAACGCC | |
| | QSYNQYYVFHMP | ATCAGTTTCAACTTCTTCCAAAGCTATAATCAATACTA | |
| | RCLFAGPLAEQFL | TGTATTCCATATGCCTCGATGTCTCTTTGCGGGTCCTCT | |
| | NQVDLTETLERYQ | GGCGGAGCAGTTTCTGAACCAGGTAGATCTGACCGAA | |
| | QRLNTYALVSKDL | ACCCTGGAAAGATACCAACAGAGACTTAACACTTACG | |
| | ASYRSFSQQLKAQ | CGCTGGTATCCAAAGACCTGGCCAGCTACCGATCTTTC | |
| | DSLGEQPTTVPPPI | TCGCAGCAGCTAAAGGCACAAGACAGCCTAGGTGAAC | |
| | DLSIPHVWMPPQT | AGCCCACCACTGTGCCACCGCCCATTGACCTGTCAATA | |
| | TPHGWTESHTTSG | CCTCACGTTTGGATGCCACCGCAAACCACTCCACACGG | |
| | LHRPHFNQTCILFD | CTGGACAGAATCACATACCACCTCAGGACTACACCGA | |
| | GHDLLFSTVTPCL | CCACACTTTAACCAGACCTGTATCCTCTTTGATGGACA | |
| | HQGFYLIDELRYV | CGATCTACTATTCAGCACCGTCACACCTTGTTTGCACC | |
| | KITLTEDFFVVTVS | AAGGCTTTTACCTCATCGACGAACTACGTTACGTTAAA | |
| | IDDDTPMLLIFGHL | ATAACACTGACCGAGGACTTCTTCGTAGTTACGGTGTC | |
| | PRVIFKAPYQRDN | CATAGACGACGACACACCCATGCTGCTTATCTTCGGCC | |
| | PILRQTEKHELLVL | ATCTTCCACGCGTACTTTTCAAAGCGCCCTATCAACGC | |
| | VKKDQLNRHSYL | GACAACTTTATACTACGACAAACTGAGAAACACGAGC | |
| | KDPDFLDAALDFN | TCCTGGTGCTAGTTAAGAAAGATCAACTGAACCGTCA | |
| | YLDISALLRNSFH | CTCTTATCTCAAAGACCCGGACTTTCTTGACGCCGCAC | |
| | RYAVDVLKSGRC | TTGACTTCAACTACCTAGACCTCAGCGCACTACTACGT | |
| | QMLDRRTVEMAF | AACAGCTTTCACCGTTACGCCGTGGATGTACTCAAGAG | |
| | AYALALFAAARQ | CGGTCGATGTCAGATGCTGGACCGCCGCACGGTAGAA | |
| | EEAGAQVSVPRAL | ATGGCCTTCGCCTACGCATTAGCACTGTTCGCAGCAGC | |
| | DRQAALLQIQEFM | CCGACAAGAAGAGGCCGGCGCCCAAGTCTCCGTCCCA | |
| | ITCLSQTPPRTTLL | CGGGCCCTAGACCGCCAGGCCGCACTCTTACAAATAC | |
| | LYPTAVDLAKRAL | AAGAATTTATGATCACCTGCCTCTCACAAACACCACCA | |
| | WTPNQITDITSLVR | CGCACCACGTTGCTGCTGTATCCCACGGCCGTGGACCT | |
| | LVYILSKQNQQHL | GGCCAAACGAGCCCTTTGGACACCGAATCAGATCACC | |
| | IPQWALRQIADFA | GACATCACCAGCCTCGTACGCCTGGTCTACATACTCTC | |
| | IKLHKTHLASFLS | TAAACAGAATCAGCAACATCTCATCCCCCAATGGGCA | |
| | AFARQELYLMGSL | CTACGACAGATCGCCGACTTTGCCCTAAAACTACACA | |
| | VHSMLVHTTERRE | AAACGCACCTGGCCTCTTTTCTTTCAGCCTTCGCACGC | |
| | IFIVETGLCSLAEL | CAAGAACTCTACCTCATGGGCAGCCTCGTCCACTCCAT | |
| | SHFTQLLAHPHHE | GCTGGTACATACGACGGAGAGACGCGAAATCTTCATC | |
| | YLSDLYTPCSSSG | GTAGAAACGGGCCTCTGTTCATTGGCCGAGCTATCACA | |
| | RRDHSLERLTRLF | CTTTACGCAGTTGTTAGCTCATCCACACCACGAATACC | |
| | PDATVPATVPAAL | TCAGCGACCTGTACACACCCTGTTCCAGTAGCGGGCG | |
| | SILSTMQPSTLETF | ACGCGATCACTCGCTCGAACGCCTCACGCGTCTCTTCC | |
| | PDLFCLPLGESFSA | CCGATGCCACCGTCCCCGCTACCGTTCCCGCCGCCCTC | |
| | LTVSEHVSYIVTN | TCCATCCTATCTACCATGCAACCAAGCACGCTGGAAAC | |
| | QYLIKGISYPVSTT | CTTCCCCGACCTGTTTTGCTTGCCGCTCGGCGAATCCTT | |
| | VVGQSLIITQTDSQ | CTCCGCGCTGACCGTCTCCGAACACGTCAGTTATATCG | |
| | TKCELTRNMHTTH | TAACAAACCAGTACCTGATCAAAGGTATCTCCTACCCT | |
| | SITVALNISLENCA | GTCTCCACCACCGTCGTAGGCCAGAGCCTCATCATCAC | |
| | FCQSALLEYDDTQ | CCAGACGGACAGTCAAACTAAATGCGAACTGACGCGC | |
| | GVINIMYMHDSDD | AACATGCATACCACACACAGCATCACAGTGGCGCTCA | |
| | VLFALDPYNEVVV | ACATTTCGCTAGAAAACTGCGCCTTTTGCCAAAGCGCC | |
| | SSPRTHYLMLLKN | CTGCTAGAATACGACGACACGCAAGGCGTCATCAACA | |
| | GTVLEVTDVVVD | TCATGTACATGCACGACTCGGACGACGTCCTTTTCGCC | |
| | ATDSRLLMMSVY | CTGGATCCCTACAACGAAGTGGTGGTCTCATCTCCGCG | |
| | ALSAIIGIYLLYRM | AACTCACTACCTCATGCTTTTGAAGAACGGTACGGTAC | |
| | LKTC (SEQ ID NO: | TAGAAGTAACTGACGTCGTCGTGGACGCCACCGACAG | |
| | 59) | TCGTCTCCTCATGATGTCCGTCTACGCGCTATCGGCCA | |
| | | TCATCGGCATCTATCTGCTCTACCGCATGCTCAAGACA | |
| | | TGC (SEQ ID NO: 87) | |
| hCMV_gH | MRPGLPSYLIILAV | ATGCGGCCAGGCCTCCCCTCCTACCTCATCATCCTCGC | C2/ |
| dimer_v2 | CLFSHLLSSRYGA | CGTCTGTCTCTTCAGCCACCTACTTTCGTCACGATATG | no cap/ |
| | EAVSEPLDKAFHL | GCGCAGAAGCCGTATCCGAACCGCTGGACAAAGCGTT | T100 |
| | LLNTYGRPIRFLRE | TCACCTACTGCTCAACACCTACGGGAGACCCATCCGCT | |
| | NTTQCTYNSSLRN | TCCTGCGTGAAAATACCACCCAGTGTACCTACAACAG | |
| | STVVRENAISFNFF | CAGCCTCCGTAACAGCACGGTCGTCAGGGAAAACGCC | |
| | QSYNQYYVFHMP | ATCAGTTTCAACTTCTTCCAAAGCTATAATCAATACTA | |
| | RCLFAGPLAEQFL | TGTATTCCATATGCCTCGATGTCTCTTTGCGGGTCCTCT | |
| | NQVDLTETLERYQ | GGCGGAGCAGTTTCTGAACCAGGTAGATCTGACCGAA | |
| | QRLNTYALVSKDL | ACCCTGGAAAGATACCAACAGAGACTTAACACTTACG | |
| | ASYRSFSQQLKAQ | CGCTGGTATCCAAAGACCTGGCCAGCTACCGATCTTTC | |
| | DSLGEQPTTVPPPI | TCGCAGCAGCTAAAGGCACAAGACAGCCTAGGTGAAC | |
| | DLSIPHVWMPPQT | AGCCCACCACTGTGCCACCGCCCATTGACCTGTCAATA | |
| | TPHGWTESHTTSG | CCTCACGTTTGGATGCCACCGCAAACCACTCCACACGG | |
| | LHRPHFNQTCILFD | CTGGACAGAATCACATACCACCTCAGGACTACACCGA | |
| | GHDLLFSTVTPCL | CCACACTTTAACCAGACCTGTATCCTCTTTGATGGACA | |
| | HQGFYLIDELRYV | CGATCTACTATTCAGCACCGTCACACCTTGTTTGCACC | |
| | KITLTEDFFVVTVS | AAGGCTTTTACCTCATCGACGAACTACGTTACGTTAAA | |
| | IDDDTPMLLIFGHL | ATAACACTGACCGAGGACTTCTTCGTAGTTACGGTGTC | |
| | PRVLFKAPYQRDN | CATAGACGACGACACACCCATGCTGCTTATCTTCGGCC | |
| | FILRQTEKHELLVL | ATCTTCCACGCGTACTTTTCAAAGCGCCCTATCAACGC | |
| | VKKDQLNRHSYL | GACAACTTTATACTACGACAAACTGAGAAACACGAGC | |
| | KDPDFLDAALDFN | TCCTGGTGCTAGTTAAGAAAGATCAACTGAACCGTCA | |
| | YLDISALLRNSFH | CTCTTATCTCAAAGACCCGGACTTTCTTGACGCCGCAC | |
| | RYAVDVLKSGRC | TTGACTTCAACTACCTAGACCTCAGCGCACTACTACGT | |
| | QMLDRRTVEMAF | AACAGCTTTCACCGTTACGCCGTGGATGTACTCAAGAG | |
| | AYALALFAAARQ | CGGTCGATGTCAGATGCTGGACCGCCGCACGGTAGAA | |
| | EEAGAQVSVPRAL | ATGGCCTTCGCCTACGCATTAGCACTGTTCGCAGCAGC | |
| | DRQAALLQIQEFM | CCGACAAGAAGAGGCCGGCGCCCAAGTCTCCGTCCCA | |
| | ITCLSQTPPRTTLL | CGGGCCCTAGACCGCCAGGCCGCACTCTTACAAATAC | |
| | LYPTAVDLAKRAL | AAGAATTTATGATCACCTGCCTCTCACAAACACCACCA | |
| | WTPNQITDITSLVR | CGCACCACGTTGCTGCTGTATCCCACGGCCGTGGACCT | |
| | LVYILSKQNQQHL | GGCCAAACGAGCCCTTTGGACACCGAATCAGATCACC | |
| | IPQWALRQIADFA | GACATCACCAGCCTCGTACGCCTGGTCTACATACTCTC | |
| | IKLHKTHLASFLS | TAAACAGAATCAGCAACATCTCATCCCCCAATGGGCA | |
| | AFARQELYLMGSL | CTACGACAGATCGCCGACTTTGCCCTAAAACTACACA | |
| | VHSMLVHTTERRE | AAACGCACCTGGCCTCTTTTCTTTCAGCCTTCGCACGC | |
| | IFIVETGLCSLAEL | CAAGAACTCTACCTCATGGGCAGCCTCGTCCACTCCAT | |
| | SHFTQLLAHPHHE | GCTGGTACATACGACGGAGAGACGCGAAATCTTCATC | |
| | YLSDLYTPCSSSG | GTAGAAACGGGCCTCTGTTCATTGGCCGAGCTATCACA | |
| | RRDHSLERLTRLF | CTTTACGCAGTTGTTAGCTCATCCACACCACGAATACC | |
| | PDATVPATVPAAL | TCAGCGACCTGTACACACCCTGTTCCAGTAGCGGGCG | |
| | SILSTMQPSTLETF | ACGCGATCACTCGCTCGAACGCCTCACGCGTCTCTTCC | |
| | PDLFCLPLGESFSA | CCGATGCCACCGTCCCCGCTACCGTTCCCGCCGCCCTC | |
| | LTVSEHVSYIVTN | TCCATCCTATCTACCATGCAACCAAGCACGCTGGAAAC | |
| | QYLIKGISYPVSTT | CTTCCCCGACCTGTTTTGCTTGCCGCTCGGCGAATCCTT | |
| | VVGQSLIITQTDSQ | CTCCGCGCTGACCGTCTCCGAACACGTCAGTTATATCG | |
| | TKCELTRNMHTTH | TAACAAACCAGTACCTGATCAAAGGTATCTCCTACCCT | |
| | SITVALNISLENCA | GTCTCCACCACCGTCGTAGGCCAGAGCCTCATCATCAC | |
| | FCQSALLEYDDTQ | CCAGACGGACAGTCAAACTAAATGCGAACTGACGCGC | |
| | GVINIMYMHDSDD | AACATGCATACCACACACAGCATCACAGTGGCGCTCA | |
| | VLFALDPYNEVVV | ACATTTCGCTAGAAAACTGCGCCTTTTGCCAAAGCGCC | |
| | SSPRTHYLMLLKN | CTGCTAGAATACGACGACACGCAAGGCGTCATCAACA | |
| | GTVLEVTDVVVD | TCATGTACATGCACGACTCGGACGACGTCCTTTTCGCC | |
| | ATDSRLLMMSVY | CTGGATCCCTACAACGAAGTGGTGGTCTCATCTCCGCG | |
| | ALSAIIGIYLLYRM | AACTCACTACCTCATGCTTTTGAAGAACGGTACGGTAC | |
| | LKTC (SEQ ID NO: | TAGAAGTAACTGACGTCGTCGTGGACGCCACCGACAG | |
| | 1)SEQ ID NO: 59) | TCGTCTCCTCATGATGTCCGTCTACGCGCTATCGGCCA | |
| | | TCATCGGCATCTATCTGCTCTACCGCATGCTCAAGACA | |
| | | TGC (SEQ ID NO: 88) | |
| hCMV_UL128 | MSPKDLTPFLTAL | ATGAGTCCCAAAGATCTGACGCCGTTCTTGACGGCGTT | C2/ |
| | WLLLGHSRVPRVR | GTGGCTGCTATTGGGTCACAGCCGCGTGCCGCGGGTG | Cap1/ |
| | AEECCEFINVNHPP | CGCGCAGAAGAATGTTGCGAATTCATAAACGTCAACC | Tailless |
| | ERCYDFKMCNRFT | ACCCGCCGGAACGCTGTTACGATTTCAAAATGTGCAAT | C2/no |
| | VALRCPDGEVCYS | CGCTTCACCGTCGCGCTGCGGTGTCCGGACGGCGAAG | cap/T100 |
| | PEKTAEIRGIVTTM | TCTGCTACAGTCCCGAGAAAACGGCTGAGATTCGCGG | C1/cap1/ |
| | THSLTRQVVHNKL | GATCGTCACCACCATGACCCATTCATTGACACGCCAGG | T100 |
| | TSCNYNPLYLEAD | TCGTACACAACAAACTGACGAGCTGCAACTACAATCC | |
| | GRIRCGKVNDKA | GTTATACCTCGAAGCTGACGGGCGAATACGCTGCGGC | |
| | QYLLGAAGSVPYR | AAAGTAAACGACAAGGCGCAGTACCTGCTGGGCGCCG | |
| | WINLEYDKITRIVG | CTGGCAGCGTTCCCTATCGATGGATCAATCTGGAATAC | |
| | LDQYLESVKKHK | GACAAGATAACCCGGATCGTGGGCCTGGATCAGTACC | |
| | RLDVCRAKMGYM | TGGAGAGCGTTAAGAAACACAAACGGCTGGATGTGTG | |
| | LQ (SEQ ID NO: 63) | CCGCGCTAAAATGGGCTATATGCTGCAG (SEQ ID NO: | |
| | | 89) | |
| hCMV-gL | MCRRPDCGFSFSP | ATGTGCCGCCGCCCGGATTGCGGCTTCTCTTTCTCACC | C2/ |
| | GPVILLWCCLLLPI | TGGACCGGTGATACTGCTGTGGTGTTGCCTTCTGCTGC | Cap1/ |
| | VSSAAVSVAPTAA | CCATTGTTTCCTCAGCCGCCGTCAGCGTCGCTCCTACC | Tailless |
| | EKVPAECPELTRR | GCCGCCGAGAAAGTCCCCGCGGAGTGCCCCGAACTAA | C2/no |
| | CLLGEVFEGDKYE | CGCGCCGATGCTTGTTGGGTGAGGTGTTTGAGGGTGAC | cap/T100 |
| | SWLRPLVNVTGR | AAGTATGAAAGTTGGCTGCGCCCGTTGGTGAATGTTAC | C1/cap1/ |
| | DGPLSQLIRYRPVT | CGGGCGCGATGGCCCGCTATCGCAACTTATCCGTTACC | T100 |
| | PEAANSVLLDEAF | GTCCCGTTACGCCGGAGGCCGCCAACTCCGTGCTGTTG | |
| | LDTLALLYNNPDQ | GACGAGGCTTTCCTGGACACTCTGGCCCTGCTGTACAA | |
| | LRALLTLLSSDTAP | CAATCCGGATCAATTGCGGGCCCTGCTGACGCTGTTGA | |
| | RWMTVMRGYSEC | GCTCGGACACAGCGCCGCGCTGGATGACGGTGATGCG | |
| | GDGSPAVYTCVD | CGGCTACAGCGAGTGCGGCGATGGCTCGCCGGCCGTG | |
| | DLCRGYDLTRLSY | TACACGTGCGTGGACGACCTGTGCCGCGGCTACGACC | |
| | GRSIFTEHVLGFEL | TCACGCGACTGTCATACGGGCGCAGCATCTTCACGGA | |
| | VPPSLFNVVVAIR | ACACGTGTTAGGCTTCGAGCTGGTGCCACCGTCTCTCT | |
| | NEATRTNRAVRLP | TTAACGTGGTGGTGGCCATACGCAACGAAGCCACGCG | |
| | VSTAAAPEGITLFY | TACCAACCGCGCCGTGCGTCTGCCCGTGAGCACCGCTG | |
| | GLYNAVKEFCLRH | CCGCGCCCGAGGGCATCACGCTCTTTTACGGCCTGTAC | |
| | QLDPPLLRHLDKY | AACGCAGTGAAGGAATTCTGCCTGCGTCACCAGCTGG | |
| | YAGLPPELKQTRV | ACCCGCCGCTGCTACGCCACCTAGATAAATACTACGCC | |
| | NLPAHSRYGPQAV | GGACTGCCGCCCGAGCTGAAGCAGACGCGCGTCAACC | |
| | DAR (SEQ ID NO: | TGCCGGCTCACTCGCGCTATGGCCCTCAAGCAGTGGAT | |
| | 3) | GCTCGC (SEQ ID NO: 90) | |
| hCMV- | MLRLLLRHHFHCL | ATGCTGCGGCTTCTGCTTCGTCACCACTTTCACTGCCT | C2/ |
| UL130 | LLCAVWATPCLAS | GCTTCTGTGCGCGGTTTGGGCAACGCCCTGTCTGGCGT | Cap1/ |
| | PWSTLTANQNPSP | CTCCGTGGTCGACGCTAACAGCAAACCAGAATCCGTC | Tailless |
| | PWSKLTYSKPHDA | CCCGCCATGGTCTAAACTGACGTATTCCAAACCGCATG | C2/no |
| | ATFYCPFLYPSPPR | ACGCGGCGACGTTTTACTGTCCTTTTCTCTATCCCTCGC | cap/T100 |
| | SPLQFSGFQRVST | CCCCACGATCCCCCTTGCAATTCTCGGGGTTCCAGCGG | C1/Cap1/ |
| | GPECRNETLYLLY | GTATCAACGGGTCCCGAGTGTCGCAACGAGACCCTGT | T100 |
| | NREGQTLVERSST | ATCTGCTGTACAACCGGGAAGGCCAGACCTTGGTGGA | |
| | WVKKVIWYLSGR | GAGAAGCTCCACCTGGGTGAAAAAGGTGATCTGGTAC | |
| | NQTILQRMPRTAS | CTGAGCGGTCGGAACCAAACCATCCTCCAACGGATGC | |
| | KPSDGNVQISVED | CCCGAACGGCTTCGAAACCGAGCGACGGAAACGTGCA | |
| | AKIFGAHMVPKQT | GATCAGCGTGGAAGACGCCAAGATTTTTGGAGCGCAC | |
| | KLLRFVVNDGTRY | ATGGTGCCCAAGCAGACCAAGCTGCTACGCTTCGTCGT | |
| | QMCVMKLESWAH | CAACGATGGCACACGTTATCAGATGTGTGTGATGAAG | |
| | VFRDYSVSFQVRL | CTGGAGAGCTGGGCTCACGTCTTCCGGGACTACAGCG | |
| | TFTEANNQTYTFC | TGTCTTTTCAGGTGCGATTGACGTTCACCGAGGCCAAT | |
| | THPNLIV (SEQ ID | AACCAGACTTACACCTTCTGCACCCATCCCAATCTCAT | |
| | NO: 65) | CGTT (SEQ ID NO: 91) | |
| pp65 phos | MESRGRRCPEMIS | ATGGAGTCGCGCGGTCGCCGTTGTCCCGAAATGATATC | C2/ |
| mut_DX | VLGPISGHVLKAV | CGTACTGGGTCCCATTTCGGGGCACGTGCTGAAAGCC | Cap1/ |
| | FSRGDTPVLPHET | GTGTTTAGTCGCGGCGATACGCCGGTGCTGCCGCACG | Tailless |
| | RLLQTGIHVRVSQ | AGACGAGACTCCTGCAGACGGGTATCCACGTACGCGT | C2/no |
| | PSLILVSQYTPDST | GAGCCAGCCCTCGCTGATCCTGGTGTCGCAGTACACGC | cap/T100 |
| | PCHRGDNQLQVQ | CCGACTCGACGCCATGCCACCGCGGCGACAATCAGCT | C1/Cap1/ |
| | HTYFTGSEVENVS | GCAGGTGCAGCACACGTACTTTACGGGCAGCGAGGTG | T100 |
| | VNVHNPTGRSICP | GAGAACGTGTCGGTCAACGTGCACAACCCCACGGGCC | |
| | SQEPMSIYVYALP | GAAGCATCTGCCCCAGCCAAGAGCCCATGTCGATCTA | |
| | LKMLNIPSINVHH | TGTGTACGCGCTGCCGCTCAAGATGCTGAACATCCCCA | |
| | YPSAAERKHRHLP | GCATCAACGTGCACCACTACCCGTCGGCGGCCGAGCG | |
| | VADAVIHASGKQ | CAAACACCGACACCTGCCCGTAGCCGACGCTGTTATTC | |
| | MWQARLTVSGLA | ACGCGTCGGGCAAGCAGATGTGGCAGGCGCGTCTCAC | |
| | WTRQQNQWKEPD | GGTCTCGGGACTGGCCTGGACGCGTCAGCAGAACCAG | |
| | VYYTSAFVFPTKD | TGGAAAGAGCCCGACGTCTACTACACGTCAGCGTTCG | |
| | VALRHVVCAHEL | TGTTTCCCACCAAGGACGTGGCACTGCGGCACGTGGT | |
| | VCSMENTRATKM | GTGCGCGCACGAGCTGGTTTGCTCCATGGAGAACACG | |
| | QVICDQYVKVYLE | CGCGCAACCAAGATGCAGGTGATAGGTGACCAGTACG | |
| | SFCEDVPSGKLFM | TCAAGGTGTACCTGGAGTCCTTCTGCGAGGACGTGCCC | |
| | HVTLGSDVEEDLT | TCCGGCAAGCTCTTTATGCACGTCACGCTGGGCTCTGA | |
| | MTRNPQPFMRPHE | CGTGGAAGAGGACCTAACGATGACCCGCAACCCGCAA | |
| | RNGFTVLCPKNMI | CCCTTCATGCGCCCCCACGAGCGCAACGGCTTTACGGT | |
| | IKPGKISHIMLDVA | GTTGTGTCCCAAAAATATGATAATCAAACCGGGCAAG | |
| | FTSHEHFGLLCPKS | ATCTCGCACATCATGCTGGATGTGGCTTTTACCTCACA | |
| | IPGLSISGNLLMNG | CGAGCATTTTGGGCTGCTGTGTCCCAAGAGCATCCCGG | |
| | QQIFLEVQAIRETV | GCCTGAGCATCTCAGGTAACCTGTTGATGAACGGGCA | |
| | ELRQYDPVAALFF | GCAAATCTTCCTGGAGGTACAAGCGATACGCGAGACC | |
| | FDIDLLLQRGPQY | GTGGAACTGCGTCAGTACGATCCCGTGGCTGCGCTCTT | |
| | SEHPTFTSQYRIQG | CTTTTTCGATATCGACTTGTTGCTGCAGCGCGGGCCTC | |
| | KLEYRHTWDRHD | AGTACAGCGAGCACCCCACCTTCACCAGCCAGTATCG | |
| | EGAAQGDDDVWT | CATCCAGGGCAAGCTTGAGTACCGACACACCTGGGAC | |
| | SGSDSDEELVTTE | CGGCACGACGAGGGTGCCGCCCAGGGCGACGACGACG | |
| | RKTPRVTGGGAM | TCTGGACCAGCGGATCGGACTCCGACGAAGAACTCGT | |
| | ASASTSAGSASSA | AACCACCGAGCGTAAGACGCCCCGCGTCACCGGCGGC | |
| | TACTAGVMTRGR | GGAGCCATGGCGAGCGCCTCCACTTCCGCGGGCTCAG | |
| | LKAESTVAPEEDT | CATCCTCGGCGACGGCGTGCACGGCGGGCGTTATGAC | |
| | DEDSDNEIHNPAV | ACGCGGCCGCCTTAAGGCCGAGTCCACCGTCGCGCCC | |
| | FTWPPWQAGILAR | GAAGAGGACACCGACGAGGATTCCGACAACGAAATCC | |
| | NLVPMVATVQGQ | ACAATCCGGCCGTGTTCACCTGGCCGCCCTGGCAGGCC | |
| | NLKYQEFFWDAN | GGCATCCTGGCCCGCAACCTGGTGCCCATGGTGGCTAC | |
| | DIYRIFAELEGVW | GGTTCAGGGTCAGAATCTGAAGTACCAGGAGTTCTTCT | |
| | QPAAQPKRRRHR | GGGACGCCAACGACATCTACCGCATCTTCGCCGAATT | |
| | QDALPGPCIASTPK | GGAAGGCGTATGGCAGCCCGCTGCGCAACCCAAACGT | |
| | KHRG (SEQ ID NO: | CGCCGCCACCGGCAAGACGCCTTGCCCGGGCCATGCA | |
| | 71) | TCGCCTCGACGCCCAAAAAGCACCGAGGT (SEQ ID NO: | |
| | | 92) | |
| pp65 WT | MESRGRRCPEMIS | ATGGAGTCGCGCGGTCGCCGTTGTCCCGAAATGATATC | C2/ |
| | VLGPISGHVLKAV | CGTACTGGGTCCCATTTCGGGGCACGTGCTGAAAGCC | CAP1/ |
| | FSRGDTPVLPHET | GTGTTTAGTCGCGGCGATACGCCGGTGCTGCCGCACG | T100 |
| | RLLQTGIHVRVSQ | AGACGCGACTCCTGCAGACGGGTATCCACGTACGCGT | |
| | PSLILVSQYTPDST | GAGCCAGCCCTCGCTGATCCTGGTGTCGCAGTACACGC | |
| | PCHRGDNQLQVQ | CCGACTCGACGCCATGCCACCGCGGCGACAATCAGCT | |
| | HTYFTGSEVENVS | GCAGGTGCAGCACACGTACTTTACGGGCAGCGAGGTG | |
| | VNVHNPTGRSICP | GAGAACGTGTCGGTCAACGTGCACAACCCCACGGGCC | |
| | SQEPMSIYVYALP | GAAGCATCTGCCCCAGCCAAGAGCCCATGTCGATCTA | |
| | LKMLNIPSINVHH | TGTGTACGCGCTGCCGCTCAAGATGCTGAACATCCCCA | |
| | YPSAAERKHRHLP | GCATCAACGTGCACCACTACCCGTCGGCGGCCGAGCG | |
| | VADAVIHASGKQ | CAAACACCGACACCTGCCCGTAGCCGACGCTGTTATTC | |
| | MWQARLTVSGLA | ACGCGTCGGGCAAGCAGATGTGGCAGGCGCGTCTCAC | |
| | WTRQQNQWKEPD | GGTCTCGGGACTGGCCTGGACGCGTCAGCAGAACCAG | |
| | VYYTSAFVFPTKD | TGGAAAGAGCCCGACGTCTACTACACGTCAGCGTTCG | |
| | VALRHVVCAHEL | TGTTTCCCACCAAGGACGTGGCACTGCGGCACGTGGT | |
| | VCSMENTRATKM | GTGCGCGCACGAGCTGGTTTGCTCCATGGAGAACACG | |
| | QVICDQYVKVYLE | CGCGCAACCAAGATGCAGGTGATAGGTGACCAGTACG | |
| | SFCEDVPSGKLFM | TCAAGGTGTACCTGGAGTCCTTCTGCGAGGACGTGCCC | |
| | HVTLGSDVEEDLT | TCCGGCAAGCTCTTTATGCACGTCACGCTGGGCTCTGA | |
| | MTRNPQPFMRPHE | CGTGGAAGAGGACCTAACGATGACCCGCAACCCGCAA | |
| | RNGFTVLCPKNMI | CCCTTCATGCGCCCCCACGAGCGCAACGGCTTTACGGT | |
| | IKPGKISHIMLDVA | GTTGTGTCCCAAGAATATGATAATCAAACCGGGCAAG | |
| | FTSHEHFGLLCPKS | ATCTCGCACATCATGCTGGATGTGGCTTTTACCTCACA | |
| | IPGLSISGNLLMNG | CGAGCATTTTGGGCTGCTGTGTCCCAAGAGCATCCCGG | |
| | QQIFLEVQAIRETV | GCCTGAGCATCTCAGGTAACCTGTTGATGAACGGGCA | |
| | ELRQYDPVAALFF | GCAAATCTTCCTGGAGGTACAAGCGATACGCGAGACC | |
| | FDIDLLLQRGPQY | GTGGAACTGCGTCAGTACGATCCCGTGGCTGCGCTCTT | |
| | SEHPTFTSQYRIQG | CTTTTTCGATATCGACTTGTTGCTGCAGCGCGGGCCTC | |
| | KLEYRHTWDRHD | AGTACAGCGAGCACCCCACCTTCACCAGCCAGTATCG | |
| | EGAAQGDDDVWT | CATCCAGGGCAAGCTTGAGTACCGACACACCTGGGAC | |
| | SGSDSDEELVTTE | CGGCACGACGAGGGTGCCGCCCAGGGCGACGACGACG | |
| | RKTPRVTGGGAM | TCTGGACCAGCGGATCGGACTCCGACGAAGAACTCGT | |
| | ASASTSAGRKRKS | AACCACCGAGCGTAAGACGCCCCGCGTCACCGGCGGC | |
| | ASSATACTAGVM | GGCGCCATGGCGAGCGCCTCCACTTCCGCGGGCCGCA | |
| | TRGRLKAESTVAP | AACGCAAATCAGCATCCTCGGCGACGGCGTGCACGGC | |
| | EEDTDEDSDNEIH | GGGCGTTATGACACGCGGCCGCCTTAAGGCCGAGTCC | |
| | NPAVFTWPPWQA | ACCGTCGCGCCCGAAGAGGACACCGACGAGGATTCCG | |
| | GILARNLVPMVAT | ACAACGAAATCCACAATCCGGCCGTGTTCACCTGGCC | |
| | VQGQNLKYQEFF | GCCCTGGCAGGCCGGCATCCTGGCCCGCAACCTGGTG | |
| | WDANDIYRIFAEL | CCCATGGTGGCTACGGTTCAGGGTCAGAATCTGAAGT | |
| | EGVWQPAAQPKR | ACCAGGAGTTCTTCTGGGACGCCAACGACATCTACCG | |
| | RRHRQDALPGPCI | CATCTTCGCCGAATTGGAAGGCGTATGGCAGCCCGCT | |
| | ASTPKKHRG (SEQ | GCGCAACCCAAACGTCGCCGCCACCGGCAAGACGCCT | |
| | ID NO: 82) | TGCCCGGGCCATGCATCGCCTCGACGCCCAAGAAGCA | |
| | | CCGAGGT (SEQ ID NO: 93) | |
| SE_CMV_gB_ | MESRIWCLVVCV | ATGGAAAGCCGGATCTGGTGTCTTGTGGTGTGCGTGA | C2/ |
| FL_061 | NLCIVCLGAAVSS | ATCTTTGCATCGTGTGCTTGGGTGCCGCCGTGTCATCT | CAP1/ |
| | SSTRGTSATHSHH | AGCAGCACAAGAGGCACCTCCGCCACTCACTCACACC | T100 |
| | SSHTTSAAHSRSG | ACAGCAGCCACACGACCAGCGCCGCTCACTCCAGAAG | |
| | SVSQRVTSSQTVS | CGGCTCTGTAAGCCAGAGAGTGACCAGTTCTCAAACC | |
| | HGVNETIYNTTLK | GTCAGCCACGGCGTCAATGAGACGATATATAATACAA | |
| | YGDVVGVNTTKY | CCCTGAAGTATGGAGACGTGGTGGGTGTCAATACCAC | |
| | PYRVCSMAQGTD | CAAGTACCCTTATCGCGTGTGCAGCATGGCCCAGGGC | |
| | LIRFERNIVCTSMK | ACTGACCTGATCAGATTCGAGAGAAATATCGTCTGCA | |
| | PINEDLDEGIMVV | CCTCCATGAAGCCTATCAACGAGGACCTTGACGAGGG | |
| | YKRNIVAHTFKVR | CATCATGGTTGTCTACAAGAGAAACATTGTGGCTCACA | |
| | VYQKVLTFRRSYA | CCTTCAAGGTGAGAGTGTATCAGAAAGTACTGACCTTT | |
| | YIHTTYLLGSNTE | AGGAGATCCTACGCTTACATCCACACCACGTACCTGCT | |
| | YVAPPMWEIHHIN | CGGCTCCAACACCGAGTATGTGGCTCCACCCATGTGG | |
| | SHSQCYSSYSRVIA | GAGATTCATCACATCAATTCCCACAGCCAATGTTACAG | |
| | GTVFVAYHRDSYE | TTCCTATAGCAGAGTCATTGCTGGTACCGTGTTCGTCG | |
| | NKTMQLMPDDYS | CTTACCACAGAGACAGCTATGAGAACAAGACCATGCA | |
| | NTHSTRYVTVKD | GTTGATGCCCGATGACTACTCCAATACACACTCTACAA | |
| | QWHSRGSTWLYR | GGTATGTGACAGTCAAAGATCAGTGGCACAGCCGGGG | |
| | ETCNLNCMVTITT | CAGCACCTGGCTGTACCGAGAGACATGTAATCTGAAT | |
| | ARSKYPYHFFATS | TGTATGGTGACTATCACTACAGCCAGGAGCAAATATC | |
| | TGDVVDISPFYNG | CATACCACTTCTTCGCCACTAGCACCGGGGACGTCGTG | |
| | TNRNASYFGENAD | GACATTTCCCCATTCTACAATGGCACAAACAGAAACG | |
| | KFFIFPNYTIVSDF | CCAGCTACTTCGGCGAGAATGCCGACAAGTTCTTTATA | |
| | GRPNSALETHRLV | TTCCCCAACTATACCATCGTGAGCGACTTCGGCCGCCC | |
| | AFLERADSVISWDI | CAACAGCGCCCTGGAAACCCACCGGCTCGTGGCCTTTC | |
| | QDEKNVTCQLTF | TCGAGCGGGCCGATAGCGTCATATCCTGGGACATCCA | |
| | WEASERTIRSEAE | GGACGAGAAGAATGTGACATGCCAGCTGACCTTCTGG | |
| | DSYHFSSAKMTAT | GAGGCCTCCGAGCGTACCATCCGGTCCGAGGCAGAGG | |
| | FLSKKQEVNMSDS | ACAGCTACCATTTCAGCAGCGCCAAGATGACCGCAAC | |
| | ALDCVRDEAINKL | CTTCCTCAGTAAGAAACAGGAGGTTAACATGTCTGATT | |
| | QQIFNTSYNQTYE | CTGCCCTGGACTGCGTGCGCGATGAGGCAATCAACAA | |
| | KYGNVSVFETTGG | GCTGCAGCAGATCTTCAACACATCTTACAACCAAACTT | |
| | LVVFWQGIKQKSL | ACGAGAAGTACGGCAACGTCAGCGTGTTCGAGACAAC | |
| | VELERLANRSSLN | AGGAGGCCTGGTAGTGTTCTGGCAAGGTATCAAGCAG | |
| | LTHNRTKRSTDGN | AAGAGTCTGGTGGAGCTCGAGCGACTGGCTAACCGCA | |
| | NATHLSNMESVH | GCTCCCTCAACCTGACCCATAATAGGACAAAGAGAAG | |
| | NLVYAQLQFTYDT | CACCGACGGCAACAACGCTACTCATTTGAGCAACATG | |
| | LRGYINRALAQIA | GAATCCGTGCACAACCTGGTGTATGCCCAGCTGCAGTT | |
| | EAWCVDQRRTLE | CACTTACGACACCCTGAGAGGCTACATCAATAGAGCC | |
| | VFKELSKINPSAIL | TTAGCTCAGATCGCAGAGGCTTGGTGTGTGGACCAGC | |
| | SAIYNKPIAARFM | GAAGAACTCTGGAGGTGTTCAAGGAGTTAAGTAAGAT | |
| | GDVLGLASCVTIN | CAATCCATCCGCCATCCTGTCTGCTATCTACAACAAGC | |
| | QTSVKVLRDMNV | CCATTGCCGCCAGGTTCATGGGAGATGTGCTCGGCCTG | |
| | KESPGRCYSRPVVI | GCTAGTTGTGTCACCATCAACCAGACCTCCGTGAAGGT | |
| | FNFANSSYVQYGQ | GCTGCGGGACATGAATGTGAAGGAGAGCCCCGGTCGG | |
| | LGEDNEILLGNHR | TGTTACTCCAGACCAGTGGTGATTTTCAACTTCGCCAA | |
| | TEECQLPSLKIFIA | CAGCTCCTATGTGCAGTACGGACAGCTCGGAGAGGAT | |
| | GNSAYEYVDYLF | AACGAGATCTTGCTCGGCAATCACAGAACTGAGGAGT | |
| | KRMIDLSSISTVDS | GTCAGCTGCCATCACTGAAGATATTTATTGCCGGGAAT | |
| | MIALDIDPLENTDF | TCCGCCTACGAATACGTTGACTACCTTTTCAAGAGAAT | |
| | RVLELYSQKELRS | GATCGACCTGAGCAGCATCAGCACCGTCGACAGCATG | |
| | SNVFDLEEIMREF | ATTGCTCTCGATATCGACCCTCTGGAGAACACCGACTT | |
| | NSYKQRVKYVED | TAGAGTCCTTGAGCTGTATTCACAGAAGGAGCTGAGG | |
| | KVVDPLPPYLKGL | AGCTCCAATGTGTTCGACCTGGAGGAAATCATGAGAG | |
| | DDLMSGLGAAGK | AGTTCAACTCTTACAAGCAGCGGGTGAAGTACGTGGA | |
| | AVGVAIGAVGGA | GGATAAGGTAGTGGACCCACTCCCACCATACCTGAAA | |
| | VASVVEGVATFLK | GGACTCGACGATCTCATGAGCGGACTGGGCGCAGCCG | |
| | NPPGAFTIILVAIA | GGAAGGCTGTTGGCGTCGCCATCGGAGCGGTCGGAGG | |
| | VVIITYLIYTRQRR | AGCAGTGGCTAGCGTGGTGGAGGGCGTGGCCACCTTC | |
| | LCTQPLQNLFPYL | CTGAAGAACCCTTTCGGCGCCTTTACCATCATCCTGGT | |
| | VSADGTTVTSGST | GGCCATCGCCGTGGTCATCATTACATATCTGATTTATA | |
| | KDTSLQAPPSYEE | CAAGACAGAGAAGGCTCTC1CACCCAGCCCTTCTCAGAA | |
| | SVYNSGRKGPGPP | CCTGTTCCCCTACCTGGTCAGTGCCGACGGTACAACCG | |
| | SSDASTAAPPYTN | TGACCAGCGGTAGCACCAAGGACACCTCCCTGCAGGC | |
| | EQAYQMLLALAR | ACCGCCGAGCTACGAGGAGTCCGTGTATAACAGTGGA | |
| | LDAEQRAQQNGT | AGAAAGGGCCCCGGACCGCCCAGCAGCGACGCATCCA | |
| | DSLDGRTGTQDK | CCGCCGCTCCTCCCTACACAAATGAGCAGGCCTATCAG | |
| | GQKPNLLDRLRHR | ATGTTGCTGGCTCTGGCACGCCTGGACGCCGAGCAGC | |
| | KNGYRHLKDSDE | GAGCTCAGCAGAACGGCACCGATTCCCTGGATGGACG | |
| | EENV (SEQ ID NO: | CACAGGCACACAGGACAAGGGGCAGAAGCCCAACCTC | |
| | 69) | CTCGACAGACTGAGACACCGGAAGAACGGATACAGGC | |
| | | ATCTGAAGGACTCCGATGAGGAGGAGAACGTT (SEQ | |
| | | ID NO: 94) | |
| SE_CMV_gB_ | MESRIWCLVVCV | ATGGAAAGCAGAATTTGGTGCCTCGTGGTCTGCGTGAACCTCTGTA | C2/ |
| FL_062 | NLCIVCLGAAVSS | TCGTGTGCTTAGGCGCCGCAGTTTCAAGCAGCTCCACCAGAGGTAC | CAP1/ |
| | SSTRGTSATHSHH | GTCGGCTACCCACAGCCATCACTCAAGTCACACTACAAGCGCCGCT | T100 |
| | SSHTTSAAHSRSG | CACAGCAGAAGCGGATCTGTGAGCCAGAGGGTGACCAGCTCCCAG | |
| | SVSQRVTSSQTVS | ACCGTGAGCCACGGAGTAAATGAAACCATCTACAATACCACATTGA | |
| | HGVNETIYNTTLK | AGTATGGCGACGTCGTGGGCGTGAACACGACCAAATACCCCTACA | |
| | YGDVVGVNTTKY | GGGTCTGCTCTATGGCTCAGGGCACTGACCTGATTCGGTFTGAGAG | |
| | PYRVCSMAQGTD | AAATATCGTCTGCACCAGCATGAAGCCCATTAACGAGGACCTGGAT | |
| | LIRFERNIVCTSMK | GAGGGCATCATGGTGGTATATAAACGTAACATTGTGGCCCACACCT | |
| | PINEDLDEGIMVV | TCAAAGTGAGAGTTTACCAGAAAGTGCTGACCTTCAGAAGATCCTA | |
| | YKRNIVAHTFKVR | CGCTTACATTCACACAACCTACCTGCTGGGCTCAAACACCGAATAC | |
| | VYQKVLTFRRSYA | GTGGCCCCTCCCATGTGGGAAATCCACCACATCAACTCTCACAGCC | |
| | YIHTTYLLGSNTE | AGTGCTACAGCTCTTACAGCAGGGTTATTGCCGGCACCGTCTTCGT | |
| | YVAPPMWEIHHIN | GGCCTACCACCGCGACAGTTATGAGAACAAGACCATGCAGCTGAT | |
| | SHSQCYSSYSRVIA | GCCTGACGACTACAGCAACACCCACTCTACCAGATACGTGACCGTT | |
| | GTVFVAYHRDSYE | AAGGACCAGTGGCACAGCCGGGGCTCAACCTGGCTGTATCGGGAA | |
| | NKTMQLMPDDYS | ACTTGTAACCTGAATTGCATGGTGACCATCACAACTGCCAGAAGCA | |
| | NTHSTRYVTVKD | AGTACCCCTATCACTTCTTCGCCACCAGCACTGGCGATGTGGTGGA | |
| | QWHSRGSTWLYR | TATCTCTCCCTTCTACAACGGAACCAATCGCAACGCTTCTTACTTTG | |
| | ETCNLNCMVTITT | GCGAGAACGCCGACAAGTTCTTTATCTTTCCCAACTACACCATCGTC | |
| | ARSKYPYHFFATS | AGCGACTTCGGTAGACCCAATTCTGCCCTGGAAACTCATCGACTTG | |
| | TGDVVDISPFYNG | TGGCATTCCTGGAAAGGGCCGATTCCGTGATCAGCTGGGACATTCA | |
| | TNRNASYFGENAD | GGACGAGAAGAACGTTACCTGCCAGCTCACATTTTGGGAGGCCAG | |
| | KFFIFPNYTIVSDF | CGAGAGGACCATTAGGAGCGAGGCCGAGGACAGCTACCACTTTTC | |
| | GRPNSALETHRLV | CAGTGCCAAGATGACAGCCACATTTCTCTCTAAGAAGCAGGAGGTT | |
| | AFLERADSVISWDI | AACATGTCCGACAGCGCCCTGGACTGTGTCAGAGACGAGGCCATC | |
| | QDEKNVTCQLTF | AATAAGCTGCAGCAGATCTTCAACACCAGCTACAATCAGACATATG | |
| | WEASERTIRSEAE | AGAAGTACGGCAACGTCAGCGTCTTCGAGACAACAGGCGGGCTGG | |
| | DSYHFSSAKMTAT | TCGTGTTCTGGCAGGGAATCAAACAGAAGTCCCTGGTTGAGCTGG | |
| | FLSKKQEVNMSDS | AGAGACTGGCGAACAGGAGCTCTCTGAATTTGACTCATAACAGGA | |
| | ALDCVRDEAINKL | CGAAGAGATCCACCGATGGAAACAACGCCACCCACCTGAGCAATA | |
| | QQIFNTSYNQTYE | TGGAGAGCGTCCACAATCTCGTCTACGCCCAGCTCCAATTCACCTAC | |
| | KYGNVSVFETTGG | GACACCCTGAGGGGCTATATCAACCGGGCCCTGGCCCAGATCGCC | |
| | LVVFWQGIKQKSL | GAGGCATGGTGCGTGGACCAGAGACGGACCCTGGAAGTGTTCAA | |
| | VELERLANRSSLN | GGAGCTGTCAAAGATCAACCCTTCCGCCATCCTCTCCGCCATATATA | |
| | LTHNRTKRSTDGN | ATAAGCCCATCGCCGCAAGATTCATGGGAGATGTCCTGGGTCTGGC | |
| | NATHLSNMESVH | TAGCTGCGTTACCATCAACCAGACATCAGTGAAGGTTTTGCGAGAC | |
| | NLVYAQLQFTYDT | ATGAATGTGAAGGAGTCACCCGGCCGATGTTACAGCCGCCCAGTC | |
| | LRGYINRALAQIA | GTGATCTTTAACTTCGCCAATTCCAGCTACGTCCAATACGGCCAGCT | |
| | EAWCVDQRRTLE | GGGCGAGGACAATGAAATTCTCCTGGGTAATCATAGAACCGAGGA | |
| | VFKELSKINPSAIL | GTGCCAACTCCCCTCCCTTAAGATTTTCATCGCAGGCAATAGCGCTT | |
| | SAIYNKPIAARFM | ATGAGTACGTTGACTACTTGTTTAAGAGAATGATCGATCTGAGCAG | |
| | GDVLGLASCVTIN | CATCAGCACAGTGGACTCCATGATTGCCCTTGATATCGATCCCCTG | |
| | QTSVKVLRDMNV | GAGAATACCGACTTTAGAGTGCTGGAGTTATACAGCCAGAAAGAG | |
| | KESPGRCYSRPVVI | CTGCGAAGCTCCAACGTGTTCGATCTGGAGGAAATTATGAGGGAG | |
| | FNFANSSYVQYGQ | TTTAACTCCTACAAGCAGAGAGTGAAGTACGTCGAAGACAAAGTG | |
| | LGEDNEILLGNHR | GTGGATCCACTGCCGCCTTATCTTAAAGGCCTCGACGATCTGATGA | |
| | TEECQLPSLKIFIA | GCGGACTGGGTGCCGCCGGCAAAGCTGTGGGCGTTGCCATCGGA | |
| | GNSAYEYVDYLF | GCCGTGGGCGGGGCCGTGGCCTCCGTGGTGGAAGGCGTGGCTAC | |
| | KRMIDLSSISTVDS | CTTTCTGAAGAACCCATTCGGCGCCTTTACCATTATCCTGGTGGCCA | |
| | MIALDIDPLENTDF | TTGCCGTGGTGATCATTACCTATCTCATCTACACTAGGCAGCGGAG | |
| | RVLELYSQKELRS | GCTGTGTACGCAGCCTCTGCAGAACCTGTTTCCCTACCTGGTTAGC | |
| | SNVFDLEEIMREF | GCCGACGGAACAACAGTGACATCTGGCTCTACCAAGGATACCTCTC | |
| | NSYKQRVKYVED | TGCAGGCACCTCCTTCTTACGAGGAATCCGTGTACAACTCGGGAAG | |
| | KVVDPLPPYLKGL | GAAAGGCCCCGGGCCACCTTCATCCGACGCCTCCACAGCTGCCCCG | |
| | DDLMSGLGAAGK | CCATACACTAACGAGCAGGCTTACCAGATGCTTCTCGCCCTGGCTA | |
| | AVGVAIGAVGGA | GATTGGATGCCGAGCAGCGCGCCCAACAGAACGGCACCGACAGCC | |
| | VASVVEGVATFLK | TGGACGGCCGGACAGGCACCCAGGACAAAGGGCAGAAGCCCAAT | |
| | NPPGAFTIILVAIA | CTGCTTGATAGACTGAGGCACCGGAAGAACGGGTACAGGCATCTT | |
| | VVIITYLIYTRQRR | AAGGACAGCGACGAGGAGGAGAACGTC (SEQ ID NO: 157) | |
| | LCTQPLQNLFPYL | | |
| | VSADGTTVTSGST | | |
| | KDTSLQAPPSYEE | | |
| | SVYNSGRKGPGPP | | |
| | SSDASTAAPPYTN | | |
| | EQAYQMLLALAR | | |
| | LDAEQRAQQNGT | | |
| | DSLDGRTGTQDK | | |
| | GQKPNLLDRLRHR | | |
| | KNGYRHLKDSDE | | |
| | EENV (SEQ ID NO: | | |
| | 69) | | |
| SE_CMV__gB_ | MESRIWCLVVCV | ATGGAATCCAGGATCTGGTGCCTCGTGGTCTGTGTGAA | C2/ |
| FL_063 | NLCIVCLGAAVSS | CTTGTGCATCGTGTGCTTGGGTGCCGCCGTGAGCAGTA | CAP1/ |
| | SSTRGTSATHSHH | GCAGCACCAGAGGCACCAGCGCAACACACTCACACCA | T100 |
| | SSHTTSAAHSRSG | CAGCTCCCATACCACTTCCGCCGCCCACTCCAGATCGG | |
| | SVSQRVTSSQTVS | GCTCCGTGAGCCAGAGGGTCACCAGGAGCCAGACGGT | |
| | HGVNETIYNTTLK | GTCCCACGGAGTGAATGAAACCATCTACAACACTACT | |
| | YGDVVGVNTTKY | CTGAAGTACGGAGACGTCGTCGGCGTGAATACCACTA | |
| | PYRVCSMAQGTD | AGTACCCCTACAGGGTCTGCTCTATGGCCCAAGGCAC | |
| | LIRFERNIVCTSMK | AGACCTGATCAGATTTGAAAGAAATATCGTCTGTACCT | |
| | PINEDLDEGIMVV | CCATGAAGCCCATCAATGAGGACTTAGACGAGGGCAT | |
| | YKRNIVAHTFKVR | TATGGTGGTGTATAAACGCAACATTGTGGCCCACACTT | |
| | VYQKVLTFRRSYA | TCAAGGTCAGAGTGTATCAGAAAGTGCTCACCTTCAG | |
| | YIHTTYLLGSNTE | GCGTAGCTATGCCTATATCCACACCACTTATCTCCTCG | |
| | YVAPPMWEIHHIN | GCAGCAACACCGAGTATGTTGCCCCGCCTATGTGGGA | |
| | SHSQCYSSYSRVIA | GATTCACCATATAAATAGCCATAGCCAGTGCTACAGCT | |
| | GTVFVAYHRDSYE | CCTATTCGAGAGTAATCGCCGGAACCGTTTTCGTCGCC | |
| | NKTMQLMPDDYS | TACCACAGAGACTCGTACGAGAACAAGACAATGCAGC | |
| | NTHSTRYVTVKD | TGATGCCAGATGACTATTCGAACACCCACAGCACGAG | |
| | QWHSRGSTWLYR | ATATGTCACCGTGAAAGATCAGTGGCACAGCAGGGGT | |
| | ETCNLNCMVTITT | AGTACATGGTTGTATAGGGAAACCTGCAATCTCAATTG | |
| | ARSKYPYHFFATS | CATGGTGACCATCACCACCGCCAGAAGCAAATACCCC | |
| | TGDVVDISPFYNG | TATCATTTCTTCGCTACCTCGACAGGAGACGTGGTGGA | |
| | TNRNASYFGENAD | CATATCTCCCTTTTATAATGGCACAAATAGAAATGCTA | |
| | KFFIFPNYTIVSDF | GCTACTTTGGAGAGAACGCCGACAAATTCTTCATCTTC | |
| | GRPNSALETHRLV | CCTAACTATACCATCGTGAGCGACTTTGGGCGACCTAA | |
| | AFLERADSVISWDI | CAGCGCCCTCGAGACTCACAGGCTGGTGGCTTTCTTAG | |
| | QDEKNVTCQLTF | AGAGGGCTGATAGTGTTATCTCTTGGGACATTCAGGAT | |
| | WEASERTIRSEAE | GAGAAGAACGTGACATGCCAGCTGACATTTTGGGAGG | |
| | DSYHFSSAKMTAT | CTAGCGAGCGAACCATCAGGTCCGAGGCCGAGGACAG | |
| | FLSKKQEVNMSDS | CTACCATTTCTCTAGTGCCAAGATGACCGCCACCTTCT | |
| | ALDCVRDEAINKL | TGTCAAAGAAGCAAGAGGTGAACATGTCCGACTCTGC | |
| | QQIFNTSYNQTYE | GCTGGACTGTGTCCGCGACGAGGCAATTAATAAACTG | |
| | KYGNVSVFETTGG | CAGCAGATCTTTAATACCAGCTACAACCAGACATACG | |
| | LVVFWQGIKQKSL | AGAAGTATGGCAACGTGAGCGTCTTCGAAACCACAGG | |
| | VELERLANRSSLN | CGGCCTTGTCGTCTTTTGGCAAGGCATCAAGCAGAAG | |
| | LTHNRTKRSTDGN | AGTCTGGTGGAGCTGGAAAGACTCGCCAACCGGTCAT | |
| | NATHLSNMESVH | CCCTGAATCTGACCCACAATAGGACAAAGCGCAGCAC | |
| | NLVYAQLQFTYDT | CGATGGGAACAACGCCACCCACCTGTCGAACATGGAG | |
| | LRGYINRALAQIA | TCAGTGCACAACCTGGTGTACGCCCAGCTGCAGTTCAC | |
| | EAWCVDQRRTLE | TTATGATACCCTCAGAGGCTACATTAACCGCGCACTGG | |
| | VFKELSKINPSAIL | CTCAGATCGCCGAAGCATGGTGCGTGGACCAGCGGCG | |
| | SAIYNKPIAARFM | AACCCTGGAAGTGTTTAAAGAGCTCTCCAAGATTAATC | |
| | GDVLGLASCVTIN | CTAGCGCCATCCTGAGTGCTATCTACAATAAGCCTATC | |
| | QTSVKVLRDMNV | GCCGCAAGATTTATGGGCGACGTGCTGGGACTGGCTT | |
| | KESPGRCYSRPVVI | CCTGCGTGACAATTAACCAGACCTCCGTCAAGGTGCTG | |
| | FNFANSSYVQYGQ | AGGGACATGAACGTGAAGGAGAGCCCCGGCAGATGCT | |
| | LGEDNEILLGNHR | ATAGCCGGCCAGTGGTGATCTTCAATTTCGCCAACAGC | |
| | TEECQLPSLKIFIA | TCATACGTGCAGTACGGCCAGCTCGGGGAGGATAATG | |
| | GNSAYEYVDYLF | AAATCCTGCTGGGAAATCACAGAACCGAGGAGTGTCA | |
| | KRMIDLSSISTVDS | GCTGCCCAGTCTGAAGATTTTCATCGCAGGCAACAGTG | |
| | MIALDIDPLENTDF | CCTATGAATACGTGGACTATCTGTTCAAACGCATGATC | |
| | RVLELYSQKELRS | GATCTGAGCTCTATCTCCACCGTGGACTCCATGATTGC | |
| | SNVFDLEEIMREF | CTTGGATATCGACCCACTGGAGAACACCGATTTCAGA | |
| | NSYKQRVKYVED | GTGCTGGAGCTGTACAGCCAGAAGGAGCTCAGGTCCA | |
| | KVVDPLPPYLKGL | GCAATGTGTTCGACCTGGAGGAAATCATGAGAGAGTT | |
| | DDLMSGLGAAGK | CAACTCCTACAAACAGAGAGTCAAGTACGTGGAGGAC | |
| | AVGVAIGAVGGA | AAGGTGGTGGATCCCCTGCCTCCCTACCTGAAGGGGCT | |
| | VASVVEGVATFLK | GGACGACCTGATGAGTGGCCTGGGAGCCGCCGGCAAA | |
| | NPFGAFTIILVAIA | GCTGTGGGAGTGGCCATCGGTGCCGTCGGAGGGGCTG | |
| | VVIITYLIYTRQRR | TGGCCAGCGTCGTCGAGGGAGTTGCCACATTCCTGAA | |
| | LCTQPLQNLFPYL | GAACCCCTTCGGGGCCTTCACCATTATCCTAGTCGCCA | |
| | VSADGTTVTSGST | TTGCCGTGGTCATCATTACCTATCTGATCTACACGCGG | |
| | KDTSLQAPPSYEE | CAGAGACGGCTGTGCACCCAGCCTTTGCAGAACCTGTT | |
| | SVYNSGRKGPGPP | CCCCTATTTAGTGTCCGCTGACGGGACCACTGTGACAA | |
| | SSDASTAAPPYTN | GCGGAAGCACCAAGGACACATCCCTGCAGGCCCCACC | |
| | EQAYQMLLALAR | CAGCTACGAGGAGTCTGTTTACAATTCTGGCCGGAAG | |
| | LDAEQRAQQNGT | GGCCCCGGCCCTCCCTCTTCTGACGCCTCCACCGCAGC | |
| | DSLDGRTGTQDK | CCCTCCTTACACAAACGAGCAGGCTTACCAGATGCTGT | |
| | GQKPNLLDRLRHR | TGGCTTTGGCCCGTCTGGACGCCGAACAGAGGGCCCA | |
| | KNGYRHLKDSDE | GCAGAATGGCACCGACTCCTTGGACGGCCGGACAGGG | |
| | EENV (SEQ ID NO: | ACCCAGGATAAGGGTCAGAAGCCTAACCTACTGGATC | |
| | 69) | GGCTCCGCCATCGCAAGAATGGCTACAGACATCTCAA | |
| | | GGACAGCGACGAAGAAGAGAATGTG (SEQ ID NO: 95) | |
| SE_CMV_gB_ | MESRIWCLVVCV | ATGGAATCAAGAATCTGGTGTCTCGTGGTGTGCGTGA | C2/ |
| FL_064 | NLCIVCLGAAVSS | ACCTGTGTATCGTCTGTCTTGGCGCCGCCGTCTCTTCCT | CAP1/ |
| | SSTRGTSATHSHH | CAAGCACCCGGGGTACCAGTGCCACCCACTCACATCA | T100 |
| | SSHTTSAAHSRSG | CTCCTCCCACACTACCAGCGCCGCCCACAGCAGATCCG | |
| | SVSQRVTSSQTVS | GCTCCGTGTCCCAGCGGGTGACCAGCAGCCAGACCGT | |
| | HGVNETIYNTTLK | GTCACACGGCGTTAATGAAACCATTTACAACACCACA | |
| | YGDVVGVNTTKY | CTGAAGTACGGGGACGTGGTGGGCGTGAACACCACCA | |
| | PYRVCSMAQGTD | AGTATCCCTACAGGGTGTGCAGCATGGCCCAGGGCAC | |
| | LIRFERNIVCTSMK | CGACCTGATTCGGTTCGAGAGAAACATCGTGTGCACA | |
| | PINEDLDEGIMVV | TCCATGAAGCCTATCAATGAGGACCTCGACGAGGGCA | |
| | YKRNIVAHTFKVR | TCATGGTGGTTTACAAGAGGAACATTGTCGCACACAC | |
| | VYQKVLTFRRSYA | ATTTAAGGTGCGAGTGTACCAGAAGGTGTTAACCTTCA | |
| | YIHTTYLLGSNTE | GAAGGTCCTACGCATACATCCACACCACCTACCTCCTG | |
| | YVAPPMWEIHHIN | GGCTCTAACACAGAATACGTCGCCCCTCCCATGTGGG | |
| | SHSQCYSSYSRVIA | AGATTCACCACATCAACAGTCACAGCCAGTGCTACAG | |
| | GTVFVAYHRDSYE | CTCGTATAGCAGAGTTATCGCTGGCACCGTGTTCGTGG | |
| | NKTMQLMPDDYS | CTTATCACCGCGACAGCTACGAGAACAAGACGATGCA | |
| | NTHSTRYVTVKD | ACTTATGCCCGACGATTACTCAAACACGCACTCCACTA | |
| | QWHSRGSTWLYR | GATACGTGACTGTGAAGGACCAGTGGCACAGTAGAGG | |
| | ETCNLNCMVTITT | CAGCACCTGGCTGTACCGGGAAACATGCAATCTCAAT | |
| | ARSKYPYHFFATS | TGTATGGTCACCATTACCACCGCCAGGTCCAAGTACCC | |
| | TGDVVDISPFYNG | TTACCACTTCTTCGCCACCTCCACTGGCGACGTGGTCG | |
| | TNRNASYFGENAD | ACATCAGCCCCTTCTACAATGGCACCAACAGGAACGC | |
| | KFFIFPNYTIVSDF | CTCTTACTTTGGGGAGAACGCCGATAAATTCTTTATTT | |
| | GRPNSALETHRLV | TCCCCAACTACACTATTGTCTCCGACTTTGGCAGACCC | |
| | AFLERADSVISWDI | AACTCAGCATTGGAAACCCACAGGCTCGTGGCCTTCCT | |
| | QDEKNVTCQLTF | GGAGCGGGCCGATAGTGTGATCAGCTGGGACATCCAG | |
| | WEASERTIRSEAE | GATGAGAAGAACGTGACATGCCAGCTGACCTTCTGGG | |
| | DSYHFSSAKMTAT | AGGCCAGCGAACGAACCATCCGGTCCGAGGCCGAGGA | |
| | FLSKKQEVNMSDS | CTCTTATCACTTCTCTAGCGCAAAGATGACCGCCACCT | |
| | ALDCVRDEAINKL | TCCTGTCTAAGAAACAGGAGGTGAACATGAGCGACAG | |
| | QQIFNTSYNQTYE | CGCCCTGGACTGCGTCAGAGACGAGGCAATCAACAAG | |
| | KYGNVSVFETTGG | CTGCAGCAAATCTTCAACACCAGCTACAACCAAACCT | |
| | LVVFWQGIKQKSL | ACGAGAAATACGGCAACGTCAGCGTCTTCGAGACTAC | |
| | VELERLANRSSLN | CGGAGGGCTCGTTGTTTTCTGGCAGGGCATTAAGCAG | |
| | ITHNRTKRSTDGN | AAGTCTCTGGTCGAGCTGGAAAGGCTGGCCAATAGAA | |
| | NATHLSNMESVH | GCTCCCTAAACCTCACTCACAACAGAACTAAGAGAAG | |
| | NLVYAQLQFTYDT | CACCGATGGCAATAACGCCACTCATCTGAGTAACATG | |
| | LRGYINRALAQIA | GAGTCTGTTCACAACCTGGTGTATGCCCAGCTGCAGTT | |
| | EAWCVDQRRTLE | TACTTATGACACACTGAGGGGCTACATCAATCGAGCC | |
| | VFKELSKINPSAIL | CTGGCCCAGATCGCCGAGGCTTGGTGCGTCGACCAGA | |
| | SAIYNKPIAARFM | GAAGAACACTGGAAGTGTTCAAGGAGCTGAGTAAGAT | |
| | GDVLGLASCVTIN | TAATCCCAGCGCCATTCTGTCCGCCATCTACAATAAGC | |
| | QTSVKVLRDMNV | CAATCGCCGCAAGATTCATGGGTGACGTGCTGGGCCT | |
| | KESPGRCYSRPVVI | GGCCTCCTGCGTGACAATCAACCAGACAAGCGTGAAA | |
| | FNFANSSYVQYGQ | GTCCTCAGAGACATGAACGTCAAGGAGTCTCCTGGCA | |
| | LGEDNEILLGNHR | GGTGTTACTCCCGGCCCGTGGTGATATTTAATTTCGCC | |
| | TEECQLPSLKIFIA | AACAGCAGTTACGTGCAGTACGGACAGCTGGGCGAGG | |
| | GNSAYEYVDYLF | ATAACGAGATACTGCTCGGAAACCATAGAACAGAGGA | |
| | KRMIDLSSISTVDS | GTGCCAACTGCCCTCCCTGAAGATTTTCATCGCCGGGA | |
| | MIALDIDPLENTDF | ACAGCGCCTATGAGTATGTTGACTATCTGTTCAAGCGG | |
| | RVLELYSQKELRS | ATGATCGACCTGAGTTCTATCAGCACCGTTGACTCCAT | |
| | SNVFDLEEIMREF | GATTGCTCTCGATATCGATCCTCTGGAGAACACCGATT | |
| | NSYKQRVKYVED | TCAGAGTGCTGGAACTCTACTCTCAGAAAGAGCTAAG | |
| | KVVDPLPPYLKGL | AAGCTCGAACGTGTTCGACCTGGAAGAAATCATGAGA | |
| | DDLMSGLGAAGK | GAGTTCAACTCCTACAAACAGAGGGTTAAGTACGTAG | |
| | AVGVAIGAVGGA | AGGATAAGGTCGTGGACCCTCTGCCTCCATACCTTAAG | |
| | VASVVEGVATFLK | GGATTAGATGATCTGATGAGCGGCCTGGGCGCTGCCG | |
| | NPFGAFTIILVAIA | GAAAGGCCGTGGGAGTGGCCATCGGCGCAGTGGGTGG | |
| | VVIITYLIYTRQRR | TGCCGTGGCTAGCGTCGTGGAAGGCGTTGCCACATTCT | |
| | LCTQPLQNLFPYL | TGAAGAACCCATTCGGGGCCTTCACAATCATCCTGGTG | |
| | VSADGTTVTSGST | GCTATCGCCGTTGTGATTATCACATATCTGATCTACAC | |
| | KDTSLQAPPSYEE | TCGCCAGCGGAGGCTCTGCACCCAGCCTCTGCAGAAC | |
| | SVYNSGRKGPGPP | CTTTTCCCCTACCTAGTGTCCGCTGATGGGACTACAGT | |
| | SSDASTAAPPYTN | CACTAGCGGCAGCACTAAGGACACATCCCTGCAGGCT | |
| | EQAYQMLLALAR | CCTCCATCTTACGAGGAGAGCGTGTATAACTCCGGGC | |
| | LDAEQRAQQNGT | GCAAGGGACCTGGCCCTCCCAGCAGCGACGCCAGCAC | |
| | DSLDGRTGTQDK | GGCGGCTCCTCCCTACACCAACGAGCAGGCATACCAG | |
| | GQKPNLLDRLRHR | ATGTTGCTTGCACTGGCCCGTCTGGACGCTGAGCAGAG | |
| | KNGYRHLKDSDE | GGCCCAGCAGAATGGGACTGATTCCCTGGACGGCAGA | |
| | EENV (SEQ ID NO: | ACCGGCACACAGGATAAAGGACAGAAACCGAATCTGC | |
| | 69) | TCGACAGGCTGAGGCACCGGAAGAATGGATACAGGCA | |
| | | TCTGAAGGACAGTGACGAGGAGGAGAACGTG (SEQ ID | |
| | | NO: 96) | |
| SE_CMV_gB_ | MESRIWCLVVCV | ATGGAGTCAAGAATCTGGTGCTTGGTGGTGTGTGTGA | C2/ |
| FL_065 | NLCIVCLGAAVSS | ACTTGTGTATCGTGTGCCTTGGAGCCGCCGTGAGCAGC | CAP1/ |
| | SSTRGTSATHSHH | AGCTCCACCAGAGGCACCAGCGCCACCCACAGCCATC | T100 |
| | SSHTTSAAHSRSG | ACTCTTCCCACACCACAAGCGCCGCCCACTCGCGGAG | |
| | SVSQRVTSSQTVS | CGGGAGTGTTTCCCAACGGGTGACAAGCAGCCAGACT | |
| | HGVNETIYNTTLK | GTGAGCCACGGCGTTAACGAGACAATCTACAACACAA | |
| | YGDVVGVNTTKY | CACTGAAGTACGGCGACGTGGTGGGTGTAAATACTAC | |
| | PYRVCSMAQGTD | CAAGTATCCTTACAGGGTGTGCTCTATGGCCCAGGGTA | |
| | LIRFERNIVCTSMK | CCGACCTGATCAGGTTTGAGAGAAACATTGTTTGCACA | |
| | PINEDLDEGIMVV | AGCATGAAGCCCATCAATGAGGACTTGGATGAGGGCA | |
| | YKRNIVAHTFKVR | TCATGGTGGTTTACAAGAGAAATATCGTGGCCCACAC | |
| | VYQKVLTFRRSYA | CTTCAAAGTGAGGGTGTATCAGAAGGTGCTGACCTTTA | |
| | YIHTTYLLGSNTE | GAAGGAGCTACGCTTATATCCACACAACCTACCTTCTG | |
| | YVAPPMWEIHHIN | GGCAGCAACACCGAGTACGTCGCACCACCCATGTGGG | |
| | SHSQCYSSYSRVIA | AAATTCACCACATCAACTCTCACTCCCAGTGCTATTCC | |
| | GTVFVAYHRDSYE | AGCTACAGCAGAGTGATAGCCGGCACAGTCTTCGTGG | |
| | NKTMQLMPDDYS | CCTACCACAGGGATAGTTACGAGAATAAGACGATGCA | |
| | NTHSTRYVTVKD | ACTGATGCCTGACGATTACTCCAACACACACAGCACC | |
| | QWHSRGSTWLYR | CGGTACGTCACCGTGAAGGACCAGTGCCACTCCAGAG | |
| | ETCNLNCMVTITT | GTAGTACTTGGCTGTACCGGGAGACTTGTAACCTGAAC | |
| | ARSKYPYHFFATS | TGCATGGTGACAATTACCACTGCTCGAAGCAAGTACC | |
| | TGDVVDISPFYNG | CTTACCACTTCTTTGCCACCTCTACCGGCGATGTCGTA | |
| | TNRNASYFGENAD | GACATATCTCCTTTCTATAACGGGACCAACAGAAACG | |
| | KFFIFPNYTIVSDF | CTTCGTACTTCGGCGAGAACGCTGACAAGTTCTTCATC | |
| | GRPNSALETHRLV | TTCCCGAACTACACTATAGTTAGCGACTTTGGTAGGCC | |
| | AFLERADSVISWDI | GAACAGCGCCCTGGAGACACACCGACTTGTGGCCTTC | |
| | QDEKNVTCQLTF | CTCGAGAGAGCTGACAGCGTGATCTCCTGGGACATCC | |
| | WEASERTIRSEAE | AGGACGAGAAGAACGTCACCTGCCAGCTGACATTCTG | |
| | DSYHFSSAKMTAT | GGAGGCCTCTGAGAGGACCATCAGATCCGAGGCCGAG | |
| | FLSKKQEVNMSDS | GATTCATACCACTTTAGCAGCGCTAAGATGACCGCTAC | |
| | ALDCVRDEAINKL | CTTCCTGAGTAAGAAGCAGGAAGTGAACATGTCCGAC | |
| | QQIFNTSYNQTYE | TCAGCCCTCGACTGCGTGAGGGACGAGGCCATCAACA | |
| | KYGNVSVFETTGG | AGCTGCAGCAGATCTTCAACACCTCCTACAACCAGAC | |
| | LVVFWQGIKQKSL | ATATGAGAAGTATGGTAACGTGAGCGTGTTCGAGACA | |
| | VELERLANRSSLN | ACCGGCGGACTGGTCGTGTTTTGGCAGGGCATAAAGC | |
| | LTHNRTKRSTDGN | AGAAGTCTCTGGTCGAGCTGGAGAGGCTGGCGAACAG | |
| | NATHLSNMESVH | GAGCAGCCTCAACCTGACCCATAACAGAACCAAACGC | |
| | NLVYAQLQFTYDT | AGCACCGACGGCAACAATGCTACCCACCTGTCAAACA | |
| | LRGYINRALAQIA | TGGAGAGCGTCCACAACCTGGTGTATGCCCAGCTGCA | |
| | EAWCVDQRRTLE | ATTTACATACGACACGCTGCGCGGCTACATCAATAGA | |
| | VFKELSKINPSAIL | GCCCTGGCCCAGATCGCCGAGGCTTGGTGCGTTGACC | |
| | SAIYNKPIAARFM | AGCGGCGTACTCTGGAAGTCTTCAAGGAGCTGAGCAA | |
| | GDVLGLASCVTIN | GATCAATCCCAGCGCTATCCTGAGCGCGATCTACAATA | |
| | QTSVKVLRDMNV | AACCTATTGCTGCCAGATTCATGGGAGACGTGTTGGG | |
| | KESPGRCYSRPVVI | GCTGGCCAGCTGCGTGACAATCAATCAGACCAGCGTG | |
| | FNFANSSYVQYGQ | AAAGTGCTGAGAGACATGAATGTGAAGGAGTCTCCTG | |
| | LGEDNEILIGNHR | GTAGGTGCTACTCAAGGCCCGTCGTAATTTTCAATTTC | |
| | TEECQLPSLKIFIA | GCCAACAGTTCCTACGTGCAGTACGGACAGCTGGGCG | |
| | GNSAYEYVDYLF | AAGACAATGAGATCCTCCTGGGCAACCATCGGACGGA | |
| | KRMIDLSSISTVDS | GGAGTGTCAACTCCCATCACTGAAGATCTTTATCGCAG | |
| | MIALDIDPLEINTDF | GCAATTCCGCCTATGAGTATGTGGACTATCTGTTCAAG | |
| | RVLELYSQKELRS | AGGATGATCGACCTGTCCAGCATCAGCACAGTGGATT | |
| | SNVFDLEEIMREF | CAATGATTGCCCTTGACATCGACCCTCTTGAGAATACC | |
| | NSYKQRVKYVED | GACTTTAGAGTGCTGGAGCTTTATAGCCAGAAAGAGC | |
| | KVVDPLPPYLKGL | TCAGGAGCTCCAATGTGTTCGACCTGGAAGAGATCAT | |
| | DDLMSGLGAAGK | GCGGGAGTTTAACAGCTACAAGCAGAGGGTTAAATAT | |
| | AVGVAIGAVGGA | GTGGAGGACAAGGTTGTGGATCCACTGCCGCCCTACC | |
| | VASVVEGVATFLK | TGAAAGGGCTGGACGACCTCATGTCCGGCCTAGGAGC | |
| | NPFGAFTIILVAIA | CGCAGGGAAAGCCGTGGGCGTGGCCATCGGCGCAGTT | |
| | VVIITYLIYTRQRR | GGAGGCGCCGTCGCCTCTGTGGTTGAAGGCGTTGCGA | |
| | LCTQPLQNLFPYL | CCTTTCTGAAGAACCCATTCGGCGCCTTCACCATTATC | |
| | VSADGTTVTSGST | CTGGTGGCCATTGCCGTGGTCATCATCACCTATCTGAT | |
| | KDTSLQAPPSYEE | CTACACCAGGCAACGACGCCTGTGCACCCAGCCCCTG | |
| | SVYNSGRKGPGPP | CAGAACCTGTTCCCTTACCTGGTCAGCGCCGATGGGAC | |
| | SSDASTAAPPYTN | CACAGTGACCTCTGGTTCTACTAAAGACACCAGCCTTC | |
| | EQAYQMLLALAR | AGGCCCCTCCATCCTACGAGGAGTCTGTGTACAATAGC | |
| | LDAEQRAQQNGT | GGCAGAAAGGGCCCCGGCCCGCCCAGCAGCGATGCCA | |
| | DSLDGRTGTQDK | GCACCGCCGCTCCTCCATACACGAACGAGCAGGCCTA | |
| | GQKPNLLDRLRHR | TCAGATGCTGCTGGCCCTTGCCCGCCTGGACGCCGAGC | |
| | KNGYRHLKDSDE | AGCGTGCTCAGCAGAATGGCACCGATTCTCTGGACGG | |
| | EENV (SEQ ID NO: | CCGAACTGGAACGCAAGACAAGGGACAGAAGCCAAA | |
| | 69) | CCTGCTGGACAGACTGAGACACAGGAAGAATGGCTAC | |
| | | AGGCATCTGAAGGATTCAGACGAGGAGGAGAACGTG | |
| | | (SEQ ID NO: 97) | |
| SE_CMV_gB_ | MESRIWCLVVCV | ATGGAGAGCCGGATCTGGTGCCTTGTGGTGTGCGTGA | C2/ |
| FL_066 | NLCIVCLGAAVSS | ACCTTTGCATCGTGTGCCTCGGCGCCGCCGTGAGCTCA | CAP1/ |
| | SSTRGTSATHSHH | TCGAGCACCCGGGGCACCAGCGCCACCCACAGCCACC | T100 |
| | SSHTTSAAHSRSG | ACAGCAGCCACACCACCAGCGCGGCCCACAGTCGGAG | |
| | SVSQRVTSSQTVS | CGGCAGCGTGAGCCAGCGGGTGACCTCCTCCCAGACC | |
| | HGVNETIYNTTLK | GTCTCCCACGGCGTGAACGAAACCATCTACAACACCA | |
| | YGDVVGVNTTKY | CCCTGAAGTACGGCGACGTGGTTGGGGTAAATACCAC | |
| | PYRVCSMAQGTD | TAAGTACCCCTACCGGGTGTGCAGCATGGCCCAGGGC | |
| | LIRFERNIVCTSMK | ACCGACCTGATCCGGTTCGAGCGGAACATCGTCTGTAC | |
| | PINEDLDEGIMVV | CAGCATGAAGCCCATCAACGAGGACCTGGACGAGGGC | |
| | YKRNIVAHTFKVR | ATCATGGTTGTCTACAAGCGGAATATCGTAGCCCACAC | |
| | VYQKVLTFRRSYA | CTTCAAGGTGCGGGTGTACCAGAAGGTGCTGACCTTCC | |
| | YIHTTYLLGSNTE | GGCGGAGCTACGCCTACATTCACACGACTTACCTGCTG | |
| | YVAPPMWEIHHIN | GGCAGCAACACCGAGTACGTGGCGCCGCCCATGTGGG | |
| | SHSQCYSSYSRVIA | AGATCCACCACATCAACTCTCACTCTCAGTGCTACAGC | |
| | GTVFVAYHRDSYE | AGCTACAGCCGGGTGATCGCCGGCACCGTGTTCGTGG | |
| | NKTMQLMPDDYS | CCTACCACCGGGACAGCTACGAGAACAAGACCATGCA | |
| | NTHSTRYVTVKD | GCTGATGCCCGACGACTATTCTAACACACACTCCACTA | |
| | QWHSRGSTWLYR | GGTACGTGACCGTGAAGGACCAGTGGCACTCCAGAGG | |
| | ETCNLNCMVTITT | CAGCACCTGGCTGTACCGGGAGACGTGCAACCTGAAC | |
| | ARSKYPYHFFATS | TGCATGGTGACCATCACCACCGCCCGGTCAAAGTACC | |
| | TGDVVDISPFYNG | CTTACCACTTCTTCGCCACCAGCACTGGGGATGTGGTT | |
| | TNRNASYFGENAD | GACATCAGCCCCTTCTACAACGGCACCAACCGGAACG | |
| | KFFIFPNYTIVSDF | CCAGCTACTTCGGCGAGAACGCCGACAAGTTCTTCATC | |
| | GRPNSALETHRLV | TTCCCCAACTACACCATCGTGAGCGACTTCGGCCGGCC | |
| | AFLERADSVISWDI | CAACAGCGCCCTGGAAACACACCGGCTGGTGGCCTTC | |
| | QDEKNVTCQLTF | CTGGAGCGGGCCGACAGCGTGATCAGCTGGGACATCC | |
| | WEASERTIRSEAE | AGGACGAGAAGAACGTGACCTGCCAACTCACATTTTG | |
| | DSYHFSSAKMTAT | GGAGGCCAGCGAGCGGACCATCCGGAGCGAGGCCGA | |
| | FLSKKQEVNMSDS | GGACTCCTATCACTTCAGCAGCGCCAAGATGACCGCC | |
| | ALDCVRDEAINKL | ACCTTCCTGAGCAAGAAGCAGGAGGTGAACATGAGCG | |
| | QQIFNTSYNQTYE | ATTCGGCATTGGACTGCGTGCGGGACGAGGCCATCAA | |
| | KYGNVSVFETTGG | CAAGCTGCAGCAGATCTTCAACACCAGCTACAACCAG | |
| | LVVFWQGIKQKSL | ACCTATGAGAAATACGGCAACGTGAGCGTGTTCGAAA | |
| | VELERLANRSSLN | CCACCGGCGGACTGGTTGTTTTCTGGCAGGGTATCAAG | |
| | LTHNRTKRSTDGN | CAGAAGAGTTTGGTGGAGCTCGAGCGCCTGGCAAACA | |
| | NATHLSNMESVH | GGAGCAGCCTGAACCTGACCCACAACCGGACCAAGCG | |
| | NLVYAQLQFTYDT | GAGCACCGACGGCAACAATGCAACGCACCTATCCAAC | |
| | LRGYINRALAQIA | ATGGAGTCCGTGCACAACCTGGTGTACGCCCAGCTGC | |
| | EAWCVDQRRTLE | AGTTCACCTACGACACCCTGCGGGGCTACATCAACCG | |
| | VFKELSKINPSAIL | GGCCCTGGCCCAGATCGCCGAGGCATGGTGCGTGGAC | |
| | SAIYNKPIAARFM | CAGCGGCGGACCCTGGAGGTGTTCAAGGAGCTTTCCA | |
| | GDVLGLASCVTIN | AGATCAACCCCTCTGCCATCCTGTCTGCTATCTACAAT | |
| | QTSVKVLRDMNV | AAGCCAATCGCGGCACGCTTCATGGGAGACGTACTGG | |
| | KESPGRCYSRPVVI | GCCTGGCCAGCTGCGTGACTATTAATCAGACTAGCGTC | |
| | FNFANSSYVQYGQ | AAAGTGCTACGGGACATGAACGTAAAGGAGAGCCCCG | |
| | LGEDNEILLGNHR | GCCGGTGCTATTCTCGGCCCGTGGTCATTTTCAACTTC | |
| | TEECQLPSLKIFIA | GCCAACAGTTCCTACGTGCAGTACGGACAGTTAGGCG | |
| | GNSAYEYVDYLF | AGGACAACGAGATTCTGCTGGGTAACCACCGGACCGA | |
| | KRMIDLSSISTVDS | GGAGTGCCAACTTCCCAGTCTAAAGATATTTATCGCCG | |
| | MIALDIDPLENTDF | GGAATTCCGCTTATGAGTATGTCGACTACCTGTTCAAG | |
| | RVLELYSQKELRS | CGGATGATCGACCTGTCCAGTATCAGCACCGTGGACA | |
| | SNVFDLEEIMREF | GCATGATTGCACTGGATATCGACCCTCTCGAGAACACC | |
| | NSYKQRVKYVED | GACTTCCGGGTGCTGGAGCTGTACAGCCAGAAAGAGC | |
| | KVVDPLPPYLKGL | TGAGATCAAGTAATGTCTTTGACCTGGAGGAGATCAT | |
| | DDLMSGLGAAGK | GCGGGAGTTCAATAGCTACAAGCAGAGGGTGAAATAT | |
| | AVGVAIGAVGGA | GTCGAAGACAAGGTAGTAGACCCGCTGCCTCCCTACC | |
| | VASVVEGVATFLK | TGAAGGGGCTTGACGACCTCATGTCAGGGTTAGGGGC | |
| | NPFGAFTIILVAIA | AGCTGGCAAGGCCGTTGGCGTCGCCATCGGCGCGGTG | |
| | VVIITYLIYTRQRR | GGCGGTGCCGTTGCCTCCGTGGTCGAAGGCGTCGCTAC | |
| | LCTQPLQNLFPYL | CTTCCTCAAGAACCCCTTCGGCGCCTTCACCATCATCC | |
| | VSADGTTVTSGST | TGGTGGCTATTGCAGTTGTCATCATTACCTACCTCATC | |
| | KDTSLQAPPSYEE | TACACCCGGCAGCGGAGGCTGTGCACCCAGCCCCTGC | |
| | SVYNSGRKGPGPP | AGAACCTGTTTCCATACCTGGTGAGCGCAGACGGAAC | |
| | SSDASTAAPPYTN | TACCGTGACGAGCGGATCCACTAAGGACACCAGCCTG | |
| | EQAYQMLLALAR | CAGGCGCCTCCTTCATACGAAGAGAGTGTGTACAACA | |
| | LDAEQRAQQNGT | GCGGCCGGAAGGGCCCCGGACCTCCGAGTAGCGACGC | |
| | DSLDGRTGTQDK | AAGTACCGCCGCCCCACCCTATACCAACGAGCAAGCT | |
| | GQKPNLLDRLRHR | TACCAGATGCTGCTGGCACTTGCTCGGCTGGACGCCGA | |
| | KNGYRHLKDSDE | ACAACGCGCCCAGCAGAACGGAACTGATTCTCTGGAC | |
| | EENV (SEQ ID NO: | GGCCGGACCGGCACCCAGGACAAGGGCCAGAAGCCC | |
| | 69) | AACCTGTTGGACCGGCTGCGGCACCGGAAGAACGGCT | |
| | | ATCGTCACCTGAAAGACAGCGACGAGGAGGAGAACGT | |
| | | G (SEQ ID NO: 98) | |
| SE_CMV_gB_ | MESRIWCLVVCV | ATGGAGAGCCGGATCTGGTGCCTAGTGGTGTGCGTGA | C2/ |
| FL_067 | NLCIVCLGAAVSS | ACCTCTGCATCGTGTGCCTAGGCGCCGCCGTGAGCAGT | CAP1/ |
| | SSTRGTSATHSHH | TCTAGTACCCGGGGCACCAGCGCCACCCACAGCCACC | T100 |
| | SSHTTSAAHSRSG | ACAGCAGCCACACTACGTCAGCAGCGCATAGTCGGAG | |
| | SVSQRVTSSQTVS | CGGCAGCGTGAGCCAGCGGGTGACGTCTTCCCAGACA | |
| | HGVNETIYNTTLK | GTGTCCCACGGCGTGAACGAAACCATCTACAACACCA | |
| | YGDVVGVNTTKY | CCCTGAAGTACGGCGACGTGGTGGGTGTCAATACAAC | |
| | PYRVCSMAQGTD | TAAGTACCCCTACCGGGTGTGCAGCATGGCCCAGGGC | |
| | LIRFERNIVCTSMK | ACCGACCTGATCCGGTTCGAGCGGAATATTGTGTGTAC | |
| | PINEDLDEGIMVV | CAGCATGAAGCCCATCAACGAGGACCTGGACGAGGGC | |
| | YKRNIVAHTFKVR | ATCATGGTGGTATACAAGAGAAACATTGTCGCCCACA | |
| | VYQKVLTFRRSYA | CCTTCAAGGTGCGGGTGTATCAGAAGGTGCTGACCTTC | |
| | YIHTTYLLGSNTE | CGGCGGAGCTACGCCTACATTCATACGACTTACCTGCT | |
| | YVAPPMWEIHHIN | GGGCAGCAACACCGAGTACGTGGCCCCGCCCATGTGG | |
| | SHSQCYSSYSRVIA | GAGATCCACCACATCAACAGCCACTCCCAGTGCTACA | |
| | GTVFVAYHRDSYE | GCAGCTACAGCCGGGTGATCGCCGGCACCGTGTTCGT | |
| | NKTMQLMPDDYS | GGCCTACCACCGGGACAGCTACGAGAACAAGACCATG | |
| | NTHSTRYVTVKD | CAGCTGATGCCCGACGACTATAGCAATACTCACAGCA | |
| | QWHSRGSTWLYR | CACGGTACGTGACCGTGAAGGACCAGTGGCACAGCCG | |
| | ETCNLNCMVTITT | CGGCAGCACCTGGCTGTACCGGGAAACGTGCAACCTG | |
| | ARSKYPYHFFATS | AACTGCATGGTGACCATCACCACCGCCCGGTCGAAGT | |
| | TGDVVDISPFYNG | ATCCCTATCACTTCTTCGCCACCAGCACGGGCGATGTG | |
| | TNRNASYFGENAD | GTTGACATCAGCCCCTTCTACAACGGCACCAACCGGA | |
| | KFFIFPNYTIVSDF | ACGCCAGCTACTTCGGCGAGAACGCCGACAAGTTCTT | |
| | GRPNSALETHRLV | CATCTTCCCCAACTACACCATCGTGAGCGACTTCGGCC | |
| | AFLERADSVISWDI | GGCCCAACAGCGCCCTGGAAACCCACCGGCTGGTGGC | |
| | QDEKNVTCQLTF | CTTCCTGGAGCGGGCCGACAGCGTGATCAGCTGGGAC | |
| | WEASERTIRSEAE | ATCCAGGACGAGAAGAACGTGACCTGCCAGCTTACAT | |
| | DSYHFSSAKMTAT | TCTGGGAGGCCAGCGAGCGGACCATCCGGAGCGAGGC | |
| | FLSKKQEVNMSDS | CGAGGACAGTTACCACTTCTCGAGCGCCAAGATGACC | |
| | ALDCVRDEAINKL | GCCACCTTCCTGAGCAAGAAGCAGGAGGTGAACATGA | |
| | QQIFNTSYNQTYE | GCGACAGTGCTCTGGACTGCGTGCGGGACGAGGCCAT | |
| | KYGNVSVFETTGG | CAACAAGCTGCAGCAGATCTTCAACACCAGCTACAAC | |
| | LVVFWQGIKQKSL | CAGACCTATGAGAAATACGGGAACGTGAGCGTGTTCG | |
| | VELERLANRSSLN | AGACAACCGGCGGCTTAGTAGTGTTCTGGCAGGGGAT | |
| | LTHNRTKRSTDGN | CAAGCAGAAGAGTTTGGTGGAGCTCGAGCGGCTGGCG | |
| | NATHLSNMESVH | AACAGAAGCAGCCTGAACCTGACCCACAACCGGACCA | |
| | NLVYAQLQFTYDT | AGCGGAGCACCGACGGCAACAACGCAACGCACTTATC | |
| | LRGYINRALAQIA | AAACATGGAAAGTGTGCACAACCTGGTGTACGCCCAG | |
| | EAWCVDQRRTLE | CTGCAGTTCACCTACGACACCCTGCGGGGCTACATCAA | |
| | VFKELSKINPSAIL | CCGGGCCCTGGCCCAGATCGCCGAGGCGTGGTGCGTG | |
| | SAIYNKPIAARFM | GACCAGCGGCGGACCCTGGAGGTGTTCAAGGAGTTGT | |
| | GDVLGLASCVTIN | CGAAGATCAACCCTTCTGCCATCCTGTCAGCAATTTAC | |
| | QTSVKVLRDMNV | AATAAACCTATTGCCGCAAGGTTCATGGGAGATGTCCT | |
| | KESPGRCYSRPVVI | GGGCCTGGCCAGCTGCGTGACCATAAACCAGACAAGC | |
| | FNFANSSYVQYGQ | GTCAAAGTCCTCCGGGACATGAATGTGAAAGAGAGCC | |
| | LGEDNEILLGNHR | CCGGCCGGTGTTACAGTCGACCCGTGGTGATCTTTAAC | |
| | TEECQLPSLKIFIA | TTCGCCAATTCTTCTTATGTGCAGTACGGACAGCTCGG | |
| | GNSAYEYVDYLF | CGAGGACAACGAGATCCTGCTCGGTAACCACCGGACC | |
| | KRMIDLSSISTVDS | GAGGAGTGTCAGCTTCCCTCACTGAAGATTTTCATTGC | |
| | MIALDIDPLENTDF | GGGGAACAGTGCATACGAGTATGTTGACTACCTGTTC | |
| | RVLELYSQKELRS | AAGCGGATGATCGATCTGTCTAGTATCAGCACCGTGG | |
| | SNVFDLEEIMREF | ACAGCATGATCGCTCTGGATATCGACCCATTGGAGAA | |
| | NSYKQRVKYVED | CACCGACTTCCGGGTGCTGGAGCTGTACAGCCAGAAG | |
| | KVVDPLPPYLKGL | GAGCTTCGCAGCAGTAATGTGTTTGACCTGGAGGAGA | |
| | DDLMSGLGAAGK | TCATGCGGGAGTTCAATTCTTACAAGCAGCGCGTGAA | |
| | AVGVAIGAVGGA | ATACGTTGAGGACAAGGTGGTCGATCCGCTGCCTCCCT | |
| | VASVVEGVATFLK | ACCTGAAGGGCCTGGATGATCTCATGAGCGGGTTAGG | |
| | NPFGAFIILVAIA | GGCTGCCGGCAAGGCCGTCGGCGTTGCCATCGGCGCA | |
| | VVIITYLIYTRQRR | GTGGGCGGAGCCGTCGCCAGCGTGGTGGAGGGTGTTG | |
| | LCTQPLQNLFPYL | CAACGTTCCTGAAGAACCCCTTCGGCGCCTTCACCATC | |
| | VSADGTTVTSGST | ATCTTGGTTGCAATCGCGGTTGTTATCATTACCTACCTT | |
| | KDTSLQAPPSYEE | ATCTACACCCGGCAACGGCGGCTGTGCACCCAGCCCC | |
| | SVYNSGRKGPGPP | TGCAGAACCTGTTTCCATACTTGGTGAGCGCGGATGGG | |
| | SSDASTAAPPYTN | ACCACCGTGACTTCAGGTTCCACCAAGGACACCAGCC | |
| | EQAYQMLLALAR | TGCAGGCGCCTCCCTCATACGAGGAGTCCGTATACAA | |
| | LDAEQRAQQNGT | CAGCGGCCGGAAGGGGCCAGGTCCTCCTAGCTCGGAC | |
| | DSLDGRTGTQDK | GCAAGTACTGCCGCACCGCCTTATACCAACGAGCAGG | |
| | GQKPNLLDRLRHR | CATATCAGATGCTGCTTGCCCTGGCTCGGCTGGACGCC | |
| | KNGYRHLKDSDE | GAACAGCGCGCCCAGCAGAACGGAACAGATTCCCTGG | |
| | EENV (SEQ ID NO: | ACGGCCGGACCGGCACCCAGGATAAGGGCCAGAAGCC | |
| | 69) | CAACTTGCTGGACCGGCTGCGGCACCGGAAGAACGGC | |
| | | TATAGGCATCTGAAGGACAGCGACGAGGAGGAGAAC | |
| | | GTG (SEQ ID NO: 99) | |
| SE_CMV_gB_ | MESRIWCLVVCV | ATGGAGAGCCGGATCTGGTGCCTAGTGGTGTGCGTGA | C2/ |
| FL_068 | NLCIVCLGAAVSS | ACCTATGCATCGTGTGCTTAGGCGCCGCCGTGAGCTCA | CAP1/ |
| | SSTRGTSATHSHH | TCGTCCACCCGGGGCACCAGCGCCACCCACAGCCACC | T100 |
| | SSHTTSAAHSRSG | ACAGCAGCCACACGACAAGCGCCGCCCACTCCCGGAG | |
| | SVSQRVTSSQTVS | CGGCAGCGTGAGCCAGCGGGTGACTTCTTCCCAGACA | |
| | HGVNETIYNTTLK | GTGAGCCACGGCGTGAACGAGACTATCTACAACACCA | |
| | YGDVVGVNTTKY | CCCTGAAGTACGGCGACGTGGTGGGCGTCAACACTAC | |
| | PYRVCSMAQGTD | CAAGTACCCCTACCGGGTGTGCAGCATGGCCCAGGGC | |
| | LIRFERNIVCTSMK | ACCGACCTGATCCGGTTCGAGCGGAACATTGTGTGCA | |
| | PINEDLDEGIMVV | CCAGCATGAAGCCCATCAACGAGGACCTGGACGAGGG | |
| | YKRNIVAHTFKVR | CATCATGGTTGTGTACAAGCGTAATATCGTCGCCCACA | |
| | VYQKVLTFRRSYA | CCTTCAAGGTGCGGGTGTACCAGAAGGTGCTGACCTTC | |
| | YIHTTYLLGSNTE | CGGCGGAGCTACGCCTACATCCATACTACGTACCTGCT | |
| | YVAPPMWEIHHIN | GGGCAGCAACACCGAGTACGTGGCTCCTCCCATGTGG | |
| | SHSQCYSSYSRVIA | GAGATCCACCACATCAACTCCCATAGCCAGTGCTACA | |
| | GTVFVAYHRDSYE | GCAGCTACAGCCGGGTGATCGCCGGCACCGTGTTCGT | |
| | NKTMQLMPDDYS | GGCCTACCACCGGGACAGCTACGAGAACAAGACCATG | |
| | NTHSTRYVTVKD | CAGCTGATGCCCGACGACTATTCGAACACCCACTCAA | |
| | QWHSRGSTWLYR | CCAGATACGTGACCGTGAAGGACCAGTGGCATTCACG | |
| | ETCNLNCMVTITT | GGGCAGCACCTGGCTGTACCGGGAAACATGCAACCTG | |
| | ARSKYPYHFFATS | AACTGCATGGTGACCATCACCACCGCCCGGAGTAAAT | |
| | TGDVVDISPFYNG | ACCCTTATCACTTCTTCGCCACCAGCACGGGCGACGTC | |
| | TNRNASYFGENAD | GTAGACATCAGCCCCTTCTACAACGGCACCAACCGGA | |
| | KFFIFPNYTIVSDF | ACGCCAGCTACTTCGGCGAGAACGCCGACAAGTTCTT | |
| | GRPNSALETHRLV | CATCTTCCCCAACTACACCATCGTGAGCGACTTCGGCC | |
| | AFLERADSVISWDI | GGCCCAACAGCGCCCTGGAGACACACCGGCTGGTGGC | |
| | QDEKNVTCQLTF | CTTCCTGGAGCGGGCCGACAGCGTGATCAGCTGGGAC | |
| | WEASERTIRSEAE | ATCCAGGACGAGAAGAACGTGACCTGCCAGCTGACGT | |
| | DSYHFSSAKMTAT | TTTGGGAGGCCAGCGAGCGGACCATCCGGAGCGAGGC | |
| | FLSKKQEVNMSDS | CGAAGATTCCTATCACTTTAGCAGCGCCAAGATGACC | |
| | ALDCVRDEAINKL | GCCACCTTCCTGAGCAAGAAGCAGGAGGTGAACATGT | |
| | QQIFNTSYNQTYE | CTGATTCCGCGCTGGACTGCGTGCGGGACGAGGCCAT | |
| | KYGNVSVFETTGG | CAACAAGCTGCAGCAGATCTTCAACACCAGCTACAAC | |
| | LVVFWQGIKQKSL | CAGACCTATGAGAAGTATGGGAACGTGAGCGTGTTCG | |
| | VELERLANRSSLN | AGACAACCGGCGGGCTGGTCGTCTTCTGGCAAGGCAT | |
| | LTHNRTKRSTDGN | TAAGCAGAAGTCCCTCGTGGAGCTGGAACGGCTGGCC | |
| | NATHLSNMESVH | AACCGTAGCAGCCTGAACCTGACCCACAACCGGACCA | |
| | NLVYAQLQFTYDT | AGCGGAGCACCGACGGCAACAATGCTACTCATCTATC | |
| | LRGYINRALAQIA | AAACATGGAAAGCGTGCACAACCTGGTGTACGCCCAG | |
| | EAWCVDQRRTLE | CTGCAGTTCACCTACGACACCCTGCGGGGCTACATCAA | |
| | VFKELSKINPSAIL | CCGGGCCCTGGCCCAGATCGCCGAGGCCTGGTGCGTG | |
| | SAIYNKPIAARFM | GACCAGCGGCGGACCCTGGAGGTGTTCAAGGAGCTAA | |
| | GDVLGLASCVTIN | GTAAGATCAACCCCTCCGCAATCCTGAGCGCCATCTAT | |
| | QTSVKVLRDMNV | AACAAGCCTATCGCCGCCCGGTTCATGGGCGATGTGCT | |
| | KESPGRCYSRPVVI | GGGCCTGGCCAGCTGCGTCACCATCAATCAAACTAGC | |
| | FNFANSSYVQYGQ | GTGAAGGTCCTACGGGACATGAACGTGAAAGAGAGCC | |
| | LGEDNEILLGNHR | CCGGCCGGTGCTACTCCCGGCCCGTGGTCATCTTCAAT | |
| | TEECQLPSLKIFIA | TTCGCCAACTCTTCCTATGTGCAGTACGGGCAGCTGGG | |
| | GNSAYEYVDYLF | CGAGGACAACGAGATTCTGCTGGGTAACCACCGGACC | |
| | KRMIDLSSISTVDS | GAGGAGTGCCAGCTTCCCTCCCTCAAGATTTTCATAGC | |
| | MIALDIDPLENTDF | AGGCAATTCTGCCTATGAATACGTTGACTACCTGTTCA | |
| | RVLELYSQKELRS | AGCGGATGATCGATCTCTCTAGTATCAGCACCGTGGAC | |
| | SNVFDIEEIMREF | AGCATGATTGCGTTGGACATCGACCCGTTAGAGAACA | |
| | NSYKQRVKYVED | CCGACTTCCGGGTGCTGGAGCTGTACAGCCAGAAAGA | |
| | KVVDPLPPYLKGL | ACTGCGTTCAAGCAACGTTTTCGACCTGGAGGAGATC | |
| | DDLMSGLGAAGK | ATGCGGGAGTTCAACTCTTACAAGCAGCGGGTCAAGT | |
| | AVGVAIGAVGGA | ACGTCGAGGATAAGGTCGTGGACCCGCTGCCGCCCTA | |
| | VASVVEGVATFLK | CCTGAAGGGACTGGACGATCTGATGTCCGGATTGGGA | |
| | NPFGAFTIILVAIA | GCTGCAGGAAAGGCCGTGGGAGTAGCCATCGGCGCTG | |
| | VVIITYLIYTRQRR | TTGGAGGGGCAGTGGCCAGCGTGGTCGAAGGCGTCGC | |
| | LCTQPLQNLFPYL | GACGTTCCTGAAGAACCCCTTCGGCGCCTTCACAATAA | |
| | VSADGTTVTSGST | TCTTGGTTGCCATTGCTGTCGTCATTATTACATATCTTA | |
| | KDTSLQAPPSYEE | TCTACACCCGGCAGAGAAGACTGTGCACCCAGCCCCT | |
| | SVYNSGRKGPGPP | GCAGAACCTGTTCCCTTATTTGGTGAGCGCCGACGGGA | |
| | SSDASTAAPPYTN | CAACCGTCACCTCCGGCTCAACGAAGGACACCAGCCT | |
| | EQAYQMLLALAR | GCAGGCTCCGCCTTCATATGAAGAGTCAGTATATAAC | |
| | LDAEQRAQQNGT | AGCGGCCGGAAGGGGCCAGGTCCTCCATCTAGCGACG | |
| | DSLDGRTGTQDK | CATCAACTGCCGCACCTCCGTACACCAACGAGCAGGC | |
| | GQKPNLLDRLRHR | ATACCAGATGCTGTTGGCCCTCGCACGGCTGGACGCC | |
| | KNGYRHLKDSDE | GAGCAACGCGCCCAGCAGAACGGGACGGACTCTTTGG | |
| | EENV (SEQ ID NO: | ATGGCCGGACCGGCACCCAAGACAAGGGCCAGAAGCC | |
| | 69) | CAATTTGCTGGACCGGCTGCGGCACCGGAAGAACGGC | |
| | | TATAGACATCTGAAGGACAGCGACGAGGAGGAGAAC | |
| | | GTG (SEQ ID NO: 100) | |
| SE_CMV_gB_ | MESRIWCLVVCV | ATGGAGAGCCGGATCTGGTGCCTAGTGGTGTGCGTGA | C2/ |
| FL_069 | NLCIVCLGAAVSS | ACCTATGCATCGTGTGCCTTGGCGCCGCCGTGAGCTCG | CAP1/ |
| | SSTRGTSATHSHH | TCCAGTACCCGGGGCACCTCCGCCACCCACTCCCACCA | T100 |
| | SSHTTSAAHSRSG | CTCCTCCCACACTACAAGCGCCGCCCACTCGCGCTCCG | |
| | SVSQRVTSSQTVS | GCTCCGTCTCCCAGCGCGTCACCAGTTCCCAGACCGTG | |
| | HGVNETIYNTTLK | AGTCACGGCGTCAACGAAACCATCTACAACACCACCC | |
| | YGDVVGVNTTKY | TCAAGTACGGCGACGTCGTGGGCGTGAATACAACCAA | |
| | PYRVCSMAQGTD | GTACCCCTACCGCGTCTGCTCCATGGCCCAGGGCACCG | |
| | LIRFERNIVCTSMK | ACCTCATCCGCTTCGAGCGCAACATCGTCTGCACCTCC | |
| | PINEDLDEGIMVV | ATGAAGCCCATCAACGAGGACCTCGACGAGGGCATCA | |
| | YKRNIVAHTFKVR | TGGTCGTGTATAAGCGAAATATTGTGGCCCACACCTTC | |
| | VYQKVLTFRRSYA | AAGGTCCGCGTCTACCAGAAGGTCCTCACCTTCCGCCG | |
| | YIHTTYLLGSNTE | CTCCTACGCCTACATTCACACAACCTACCTCCTCGGCT | |
| | YVAPPMWEIHHIN | CCAACACCGAGTACGTCGCCCCTCCCATGTGGGAGAT | |
| | SHSQCYSSYSRVIA | CCACCACATCAACAGTCACAGCCAGTGCTACTCCTCCT | |
| | GTVFVAYHRDSYE | ACTCCCGCGTCATCGCCGGCACCGTCTTCGTCGCCTAC | |
| | NKTMQLMPDDYS | CACCGCGACTCCTACGAGAACAAGACCATGCAGCTCA | |
| | NTHSTRYVTVKD | TGCCCGACGACTATAGCAATACACATAGTACCCGCTA | |
| | QWHSRGSTWLYR | CGTCACCGTCAAGGACCAGTGGCACAGCAGGGGCTCC | |
| | ETCNLNCMVTITT | ACCTGGCTCTACCGCGAGACTTGCAACCTCAACTGCAT | |
| | ARSKYPYHFFATS | GGTCACCATCACCACCGCCCGCTCAAAGTACCCGTATC | |
| | TGDVVDISPFYNG | ACTTCTTCGCCACCTCCACGGGAGACGTGGTGGACATC | |
| | TNRNASYFGENAD | TCCCCTTTCTACAACGGCACCAACCGCAACGCTAGCTA | |
| | KFFIFPNYTIVSDF | TTTCGGCGAGAACGCCGACAAGTTCTTCATCTTCCCCA | |
| | GRPNSALETHRLV | ACTACACCATCGTCTCCGACTTCGGCCGCCCCAACTCC | |
| | AFLERADSVISWDI | GCCCTCGAAACCCACAGGCTTGTGGCCTTCCTCGAGCG | |
| | QDEKNVTCQLTF | CGCCGACTCCGTCATCTCCTGGGACATCCAGGACGAG | |
| | WEASERTIRSEAE | AAGAACGTCACCTGCCAGCTCACATTCTGGGAGGCCT | |
| | DSYHFSSAKMTAT | CCGAGCGCACCATCCGCTCCGAGGCCGAGGATTCGTA | |
| | FLSKKQEVNMSDS | CCACTTTAGCTCAGCAAAGATGACCGCCACCTTCCTCT | |
| | ALDCVRDEAINKL | CCAAGAAGCAGGAGGTCAACATGAGCGACTCTGCTTT | |
| | QQIFNTSYNQTYE | GGACTGCGTCCGCGACGAGGCCATCAACAAGCTCCAG | |
| | KYGNVSVFETTGG | CAGATCTTCAACACCTCCTACAACCAGACTTATGAGAA | |
| | LVVFWQGIKQKSL | GTATGGCAACGTCTCGGTGTTCGAGACTACGGGCGGT | |
| | VELERLANRSSLN | CTGGTGGTCTTCTGGCAGGGGATTAAGCAGAAGTCCCT | |
| | LTHNRTKRSTDGN | CGTCGAGTTGGAGAGACTCGCCAACCGCTCCTCCCTCA | |
| | NATHLSNMESVH | ACCTCACCCACAACCGCACCAAGCGCTCCACCGACGG | |
| | NLVYAQLQFTYDT | CAACAACGCCACGCACCTCTCAAACATGGAGTCCGTC | |
| | LRGYINRALAQIA | CACAACCTCGTCTACGCCCAGCTCCAGTTCACCTACGA | |
| | EAWCVDQRRTLE | CACCCTCCGCGGCTACATCAACCGCGCCCTCGCCCAGA | |
| | VFKELSKINPSAIL | TCGCCGAGGCCTGGTGCGTCGACCAGCGCCGCACCCT | |
| | SAIYNKPIAARFM | CGAGGTCTTCAAGGAGCTCAGTAAGATCAACCCAAGT | |
| | GDVLGLASCVTIN | GCGATCCTGTCGGCCATTTACAATAAACCGATTGCAGC | |
| | QTSVKVLRDMNV | CCGCTTCATGGGTGACGTACTCGGCCTCGCCTCCTGCG | |
| | KESPGRCYSRPVVI | TGACGATTAATCAGACCAGCGTCAAGGTGCTTCGCGA | |
| | FNFANSSYVQYGQ | CATGAATGTGAAGGAGAGCCCAGGCCGCTGTTACAGT | |
| | LGEDNEILLGNHR | CGGCCCGTCGTCATTTTCAATTTCGCCAATAGCAGCTA | |
| | TEECQLPSLKIFIA | TGTCCAGTACGGCCAGCTCGGCGAGGACAACGAGATA | |
| | GNSAYEYVDYLF | CTCCTTGGCAACCACCGCACCGAGGAGTGCCAGCTGC | |
| | KRMIDLSSISTVDS | CGTCTCTGAAGATATTCATAGCCGGCAACAGCGCTTAT | |
| | MIALDIDPLENTDF | GAATACGTGGACTACCTCTTCAAGCGCATGATCGACCT | |
| | RVLELYSQKELRS | CTCCTCCATCTCCACCGTCGACTCCATGATCGCACTTG | |
| | SNVFDLEEIMREF | ATATCGACCCACTGGAGAACACCGACTTCCGCGTTCTG | |
| | NSYKQRVKYVED | GAACTCTACTCCCAGAAGGAGCTACGGTCCTCCAATGT | |
| | KVVDPLPPYLKGL | TTTCGACCTCGAGGAGATCATGCGCGAGTTCAATTCAT | |
| | DDLMSGLGAAGK | ACAAGCAACGGGTGAAGTATGTGGAGGACAAGGTCGT | |
| | AVGVAIGAVGGA | CGATCCTCTGCCTCCCTACCTCAAGGGTCTTGATGATC | |
| | VASVVEGVATFLK | TCATGTCCGGCCTCGGCGCTGCCGGGAAGGCAGTGGG | |
| | NPFGAFTIILVAIA | AGTCGCCATCGGCGCCGTTGGAGGGGCCGTCGCCTCT | |
| | VVIITYLIYTRQRR | GTGGTGGAGGGCGTGGCTACCTTCCTGAAGAACCCCTT | |
| | LCTQPLQNLFPYL | CGGCGCCTTCACCATTATTCTGGTGGCCATCGCAGTGG | |
| | VSADGTTVTSGST | TTATCATCACGTACCTTATCTACACCCGGCAGAGAAGG | |
| | KDTSLQAPPSYEE | CTCTGCACCCAGCCCCTCCAGAACCTCTTTCCTTATCTC | |
| | SVYNSGRKGPGPP | GTCAGCGCAGACGGTACAACAGTTACTAGTGGAAGTA | |
| | SSDASTAAPPYTN | CCAAGGACACCTCCCTCCAGGCCCCGCCAAGCTACGA | |
| | EQAYQMLLALAR | GGAAAGTGTTTACAACTCCGGCCGCAAGGGCCCCGGC | |
| | LDAEQRAQQNGT | CCACCTTCTTCCGACGCTTCCACCGCTGCTCCACCATA | |
| | DSLDGRTGTQDK | CACCAACGAGCAGGCCTACCAGATGCTCCTGGCACTG | |
| | GQKPNLLDRLRHR | GCTCGGCTGGATGCCGAGCAGAGGGCCCAGCAGAACG | |
| | KNGYRHLKDSDE | GTACCGATTCCCTCGACGGCCGCACCGGCACCCAGGA | |
| | EENV (SEQ ID NO: | TAAGGGCCAGAAGCCCAATTTACTAGACAGACTGCGC | |
| | 69) | CACCGGAAGAACGGCTACCGCCATCTGAAGGACTCCG | |
| | | ACGAGGAGGAGAACGTC (SEQ ID NO: 101) | |
| SE_CMV_gB_ | MESRIWCLVVCV | ATGGAGAGCCGGATCTGGTGCCTAGTGGTGTGCGTGA | C2/ |
| FL_070 | NLCIVCLGAAVSS | ACCTCTGCATCGTGTGCTTGGGCGCCGCCGTGAGTAGT | CAP1/ |
| | SSTRGTSATHSHH | TCCAGTACCCGGGGCACCTCCGCCACCCACTCCCACCA | T100 |
| | SSHTTSAAHSRSG | CTCCTCCCACACTACGAGTGCCGCCCACTCACGCTCCG | |
| | SVSQRVTSSQTVS | GCTCCGTCTCCCAGCGCGTCACTAGTTCTCAGACAGTG | |
| | HGVNETIYNTTLK | TCTCACGGCGTCAACGAGACAATCTACAACACCACCC | |
| | YGDVVGVNTTKY | TCAAGTACGGCGACGTCGTGGGTGTGAATACTACTAA | |
| | PYRVCSMAQGTD | GTACCCCTACCGCGTCTGCTCCATGGCCCAGGGCACCG | |
| | LIRFERNIVCTSMK | ACCTCATCCGCTTCGAGCGCAACATCGTCTGCACCTCC | |
| | PINEDLDEGIMVV | ATGAAGCCCATCAACGAGGACCTCGACGAGGGCATCA | |
| | YKRNIVAHTFKVR | TGGTCGTCTACAAGAGAAATATAGTGGCCCACACCTTC | |
| | VYQKVLTFRRSYA | AAGGTCCGCGTCTACCAGAAGGTCCTCACCTTCCGCCG | |
| | YIHTTYLLGSNTE | CTCCTACGCCTACATTCACACTACCTACCTCCTCGGCT | |
| | YVAPPMWEIHHIN | CCAACACCGAGTACGTCGCCCCACCCATGTGGGAGAT | |
| | SHSQCYSSYSRVIA | CCACCACATCAACTCTCACAGTCAGTGCTACTCCTCCT | |
| | GTVFVAYHRDSYE | ACTCCCGCGTCATCGCCGGCACCGTCTTCGTCGCCTAC | |
| | NKTMQLMPDDYS | CACCGCGACTCCTACGAGAACAAGACCATGCAGCTCA | |
| | NTHSTRYVTVKD | TGCCCGACGACTACTCAAACACTCACAGCACCCGCTA | |
| | QWHSRGSTWLYR | CGTCACCGTCAAGGACCAGTGGCACAGCCGCGGCTCC | |
| | ETCNLNCMVTITT | ACCTGGCTCTACCGCGAGACTTGCAACCTCAACTGCAT | |
| | ARSKYPYHFFATS | GGTCACCATCACCACCGCCCGCTCGAAGTATCCGTACC | |
| | TGDVVDISPFYNG | ACTTCTTCGCCACCTCCACGGGCGATGTGGTCGACATC | |
| | TNRNASYFGENAD | AGTCCATTCTACAACGGCACCAACCGCAACGCTTCATA | |
| | KFFIFPNYTIVSDF | CTTCGGCGAGAACGCCGACAAGTTCTTCATCTTCCCCA | |
| | GRPNSALETHRLV | ACTACACCATCGTCTCCGACTTCGGCCGCCCCAACTCC | |
| | AFLERADSVISWDI | GCCCTCGAGACACACCGCCTGGTCGCCTTCCTCGAGCG | |
| | QDEKNVTCQLTF | CGCCGACTCCGTCATCTCCTGGGACATCCAGGACGAG | |
| | WEASERTIRSEAE | AAGAACGTCACCTGCCAGTTGACCTTCTGGGAGGCCTC | |
| | DSYHFSSAKMTAT | CGAGCGCACCATCCGCTCCGAGGCCGAGGACAGTTAC | |
| | FLSKKQEVNMSDS | CATTTCAGTAGTGCCAAGATGACCGCCACCTTCCTCTC | |
| | ALDCVRDEAINKL | CAAGAAGCAGGAGGTCAACATGTCGGACTCTGCTCTT | |
| | QQIFNTSYNQTYE | GACTGCGTCCGCGACGAGGCCATCAACAAGCTCCAGC | |
| | KYGNVSVFETTGG | AGATCTTCAACACCTCCTACAACCAGACTTATGAGAA | |
| | LVVPWQGIKQKSL | GTATGGTAACGTCTCTGTGTTTGAGACTACAGGCGGTC | |
| | VELERLANRSSLN | TTGTCGTCTTCTGGCAGGGTATCAAGCAGAAGTCCCTC | |
| | LTHNRTKRSTDGN | GTCGAGCTGGAACGCCTCGCCAACCGCTCCTCCCTCAA | |
| | NATHLSNMESVH | CCTCACCCACAACCGCACCAAGCGCTCCACCGACGGC | |
| | NLVYAQLQFTYDT | AACAACGCAACACATCTTAGCAACATGGAGTCCGTCC | |
| | LRGYINRALAQIA | ACAACCTCGTCTACGCCCAGCTCCAGTTCACCTACGAC | |
| | EAWCVDQRRTLE | ACCCTCCGCGGCTACATCAACCGCGCCCTCGCCCAGAT | |
| | VFKELSKINPSAIL | CGCCGAGGCCTGGTGCGTCGACCAGCGCCGCACCCTC | |
| | SAIYNKPIAARFM | GAGGTCTTCAAGGAGCTGAGTAAGATCAACCCAAGTG | |
| | GDVLGLASCVTIN | CAATCCTAAGCGCTATTTACAACAAACCTATCGCAGCC | |
| | QTSVKVLRDMNV | AGGTTCATGGGAGACGTCCTCGGCCTCGCCTCCTGCGT | |
| | KESPGRCYSRPVVI | CACCATTAATCAGACGTCTGTTAAGGTTCTCCGCGACA | |
| | FNFANSSYVQYGQ | TGAACGTGAAAGAGTCTCCGGGCCGCTGCTACAGCAG | |
| | LGEDNEILLGNHR | GCCCGTCGTCATCTTCAATTTCGCCAATTCTTCATATGT | |
| | TEECQLPSLKIFIA | CCAGTACGGCCAGCTCGGCGAGGACAACGAGATTCTC | |
| | GNSAYEYVDYLF | TTAGGGAACCACCGCACCGAGGAGTGTCAGCTACCCA | |
| | KRMIDLSSISTVDS | GCCTGAAGATCTTTATTGCCGGCAATAGCGCTTATGAG | |
| | MIALDIDPLENTDF | TATGTTGACTACCTCTTCAAGCGCATGATCGACCTCTC | |
| | RVLELYSQKELRS | CTCCATCTCCACCGTCGACTCCATGATCGCTCTGGATA | |
| | SNVFDLEEIMREF | TCGACCCTCTGGAGAACACCGACTTCCGCGTGCTTGAG | |
| | NSYKQRVKYVED | CTCTACTCCCAGAAAGAGCTTAGGTCAAGCAACGTTTT | |
| | KVVDPLPPYLKGL | CGACCTCGAGGAGATCATGCGCGAGTTCAACTCATAT | |
| | DDLMSGLGAAGK | AAGCAACGCGTTAAATATGTAGAGGATAAGGTGGTTG | |
| | AVGVAIGAVGGA | ATCCACTTCCTCCCTACCTCAAGGGTCTGGATGACCTC | |
| | VASVVEGVATFLK | ATGTCCGGCCTCGGGGCAGCAGGCAAGGCCGTCGGCG | |
| | NPFGAFTIILVAIA | TTGCCATCGGCGCCGTGGGAGGTGCTGTGGCCAGTGTT | |
| | VVIITYLIYTRQRR | GTCGAGGGCGTAGCCACCTTCTTAAAGAACCCCTTCGG | |
| | LCTQPLQNLFPYL | CGCCTTTACAATAATCCTGGTGGCCATCGCTGTGGTTA | |
| | VSADGTTVTSGST | TCATTACCTATCTTATCTACACCAGGCAGCGGAGGCTC | |
| | KDTSLQAPPSYEE | TGCACCCAGCCCCTCCAGAACCTCTTCCCTTACCTCGT | |
| | SVYNSGRKGPGPP | GAGCGCGGACGGGACGACCGTCACATCTGGCAGTACA | |
| | SSDASTAAPPYTN | AAGGACACCTCCCTCCAGGCCCCGCCTAGTTATGAAG | |
| | EQAYQMLLALAR | AGAGCGTTTACAACTCCGGCCGCAAGGGCCCCGGTCC | |
| | LDAEQRAQQNGT | TCCCTCCTCCGACGCCAGCACCGCAGCGCCTCCATACA | |
| | DSLDGRTGTQDK | CCAACGAGCAGGCCTACCAGATGCTCTTGGCCCTGGC | |
| | GQKPNLLDRLRHR | CCGACTGGATGCCGAGCAGCGCGCCCAGCAGAACGGA | |
| | KNGYRHLKDSDE | ACCGACTCCCTCGACGGCCGCACCGGCACCCAGGACA | |
| | EENV (SEQ ID NO: | AAGGCCAGAAGCCCAATCTGCTCGACCGCCTGCGACA | |
| | 69) | CCGCAAGAACGGCTATCGGCACCTTAAAGACTCCGAC | |
| | | GAGGAGGAGAACGTC (SEQ ID NO: 102) | |
| SE_CMV_gH_ | MRPGLPSYLIILAV | ATGCGGCCAGGACTTCCTAGCTACCTAATCATCCTTGC | C2/ |
| 031 | CLFSHLLSSRYGA | CGTGTGTCTTTTCTCACACCTCCTCTCTAGTAGGTACG | CAP1/ |
| | EAVSEPLDKAFHL | GCGCAGAGGCCGTGTCCGAGCCACTCGACAAGGCCTT | T100 |
| | LLNTYGRPIRFLRE | CCACCTTTTGTTAAACACCTACGGTCGGCCAATCAGAT | |
| | NTTQCTYNSSLRN | TCCTCCGCGAGAACACCACCCAGTGCACGTACAATTC | |
| | STVVRENAISFNFF | GTCTCTCCGCAACAGCACAGTCGTGAGGGAGAACGCT | |
| | QSYNQYYVFHMP | ATATCATTCAACTTCTTCCAGTCTTACAACCAGTATTA | |
| | RCLFAGPLAEQFL | CGTGTTCCACATGCCAAGATGCCTGTTCGCTGGCCCAC | |
| | NQVDLTETLERYQ | TGGCCGAGCAGTTCCTTAACCAGGTGGATCTGACAGA | |
| | QRLNTYALVSKDL | GACTCTGGAGAGATACCAACAGAGGCTGAACACCTAC | |
| | ASYRSFSQQLKAQ | GCACTGGTGAGCAAGGATCTGGCCTCCTACAGGAGCT | |
| | DSLGEQPTTVPPPI | TCAGCCAACAGCTGAAGGCCCAGGACAGTCTGGGCGA | |
| | DLSIPHVWMPPQT | GCAACCAACCACCGTACCTCCACCTATTGACTTATCAA | |
| | TPHGWTESHTTSG | TACCTCACGTGTGGATGCCTCCTCAGACCACTCCTCAC | |
| | LHRPHFNQTCILFD | GGCTGGACCGAATCCCACACCACCTCCGGCCTGCACA | |
| | GHDLLFSTVTPCL | GGCCTCATTTCAACCAGACCTGTATTCTCTTCGACGGA | |
| | HQGFYLIDELRYV | CACGACCTGCTTTTCAGCACCGTCACGCCATGCTTGCA | |
| | KITLTEDFFVVTVS | CCAGGGCTTCTACCTGATCGACGAGCTCAGGTATGTGA | |
| | IDDDTPMLLIFGHL | AGATCACTCTGACCGAGGACTTCTTCGTGGTTACTGTG | |
| | PRVLFKAPYQRDN | AGCATCGATGACGATACCCCAATGTTACTGATCTTCGG | |
| | FILRQTEKHELLVL | CCACCTGCCTAGGGTGCTGTTCAAGGCACCATACCAG | |
| | VKKDQLNRHSYL | AGAGACAATTTCATCCTGAGACAAACCGAGAAGCACG | |
| | KDPDFLDAALDFN | AGCTGCTGGTGCTGGTCAAGAAGGACCAGCTGAATAG | |
| | YLDLSALLRNSFH | GCACTCATACCTGAAGGACCCAGATTTCCTGGACGCTG | |
| | RYAVDVLKSGRC | CCCTTGATTTCAATTACCTGGACCTGTCCGCCCTGCTG | |
| | QMLDRRTVEMAF | AGAAACAGCTTCCACAGATACGCCGTGGATGTGCTGA | |
| | AYALALFAAARQ | AGTCAGGCAGATGTCAGATGTTGGACCGCCGAACCGT | |
| | EEAGAQVSVPRAL | TGAGATGGCCTTCGCCTACGCGCTGGCCCTGTTCGCCG | |
| | DRQAALLQIQEFM | CCGCTCGGCAGGAGGAAGCTGGCGCACAGGTGAGCGT | |
| | ITCLSQTPPRTTLL | GCCGAGGGCTCTGGACCGACAGGCTGCTCTGCTACAG | |
| | LYPTAVDLAKRAL | ATTCAGGAATTCATGATCACCTGCCTGTCACAGACTCC | |
| | WTPNQITDITSLVR | GCCTCGGACTACCCTGCTGCTGTATCCTACAGCAGTGG | |
| | LVYILSKQNQQHL | ACCTGGCAAAGAGAGCTCTTTGGACCCCTAACCAGAT | |
| | IPQWALRQIADFA | CACCGATATCACCAGCCTCGTCCGGCTGGTCTACATTC | |
| | LKLHKTHLASFLS | TGTCTAAGCAGAATCAGCAGCACCTGATCCCTCAGTG | |
| | AFARQELYLMGSL | GGCTCTCAGACAGATCGCCGATTTCGCCCTGAAGCTGC | |
| | VHSMLVHTTERRE | ACAAGACCCACCTGGCTTCTTTCCTGAGCGCTTTCGCC | |
| | IFIVETGLCSLAEL | AGACAGGAACTGTACCTGATGGGATCGCTTGTGCACA | |
| | SHFTQLLAHPHHE | GCATGCTGGTGCACACAACTGAGAGGAGAGAGATTTT | |
| | YLSDLYTPCSSSG | CATTGTGGAGACTGGCCTGTGCAGCCTGGCCGAGCTGT | |
| | RRDHSLERLTRLF | CCCATTTCACCCAGCTCCTCGCTCATCCGCACCACGAG | |
| | PDATVPATVPAAL | TACCTCTCCGACCTCTATACCCCTTGCTCCTCTTCCGGC | |
| | SILSTMQPSTLETF | CGGCGCGATCACAGCCTGGAAAGACTCACTAGACTGT | |
| | PDLFCLPLGESFSA | TCCCAGACGCTACCGTGCCGGCTACTGTCCCGGCAGCA | |
| | LTVSEHVSYIVTN | CTGAGCATCCTGAGCACTATGCAGCCTTCTACGCTGGA | |
| | QYLIKGISYPVSTT | AACCTTCCCGGACCTGTTCTGCCTGCCACTCGGAGAAA | |
| | VVGQSLIITQTDSQ | GCTTCTCTGCCCTGACGGTGAGTGAGCACGTGTCGTAC | |
| | TKCELTRNMHTTH | ATCGTGACAAACCAGTACCTGATCAAGGGTATCAGCT | |
| | SITVALNISLENCA | ACCCAGTGTCCACAACTGTGGTGGGCCAGAGCCTGAT | |
| | FCQSALLEYDDTQ | CATCACCCAGACCGATAGCCAAACAAAGTGCGAACTG | |
| | GVINIMYMHDSDD | ACAAGAAACATGCATACCACTCATTCCATCACTGTGGC | |
| | VLFALDPYNEVVV | CTTAAACATCTCCCTGGAGAACTGCGCCTTCTGTCAGT | |
| | SSPRTHYLMLLKN | CCGCCCTGCTGGAGTACGATGATACCCAGGGCGTTATT | |
| | GTVLEVTDVVVD | AATATCATGTACATGCATGATAGCGACGATGTGCTTTT | |
| | ATDSRLLMMSVY | CGCCCTGGACCCATACAACGAGGTGGTGGTGTCCAGC | |
| | ALSAIIGIYLLYRM | CCTAGAACCCACTACCTCATGCTGCTGAAGAACGGCA | |
| | LKTC (SEQ ID NO: | CAGTGCTGGAGGTGACCGACGTGGTGGTGGACGCTAC | |
| | 59) | GGACAGCAGGCTGCTGATGATGAGCGTGTACGCCCTG | |
| | | AGCGCCATTATCGGAATATACCTGCTGTACAGGATGTT | |
| | | GAAGACCTGT (SEQ ID NO: 103) | |
| SE_CMV_gH_ | MRPGLPSYLIILAV | ATGCGGCCGGGACTCCCTAGCTACCTCATCATCCTCGC | C2/ |
| 032 | CLFSHLLSSRYGA | CGTGTGCCTTTTCTCTCACCTTTTGAGCTCCAGATACG | CAP1/ |
| | EAVSEPLDKAFHL | GCGCCGAAGCGGTGTCCGAGCCTCTCGACAAGGCCTT | T100 |
| | LLNTYGRPIRFLRE | CCACCTCCTTCTCAACACATACGGCAGACCGATCCGGT | |
| | NTTQCTYNSSLRN | TCTTGAGGGAGAACACTACCCAATGCACATATAACAG | |
| | STVVRENAISFNFF | TAGCCTGCGGAATAGCACTGTGGTGCGGGAGAACGCC | |
| | QSYNQYYVFHMP | ATCAGCTTCAATTTCTTCCAGAGCTACAATCAGTATTA | |
| | RCLFAGPLAEQFL | CGTGTTCCACATGCCTAGATGTCTGTTCGCGGGCCCTC | |
| | NQVDLTETLERYQ | TGGCAGAGCAGTTCCTGAACCAGGTTGATCTCACAGA | |
| | QRLNTYALVSKDL | GACACTGGAGAGATACCAGCAAAGACTGAACACCTAC | |
| | ASYRSFSQQLKAQ | GCTCTCGTCTCCAAGGACCTGGCCAGCTATAGGAGCTT | |
| | DSLGEQPTTVPPPI | CAGCCAGCAGCTGAAGGCCCAGGATTCCCTGGGAGAG | |
| | DLSIPHVWMPPQT | CAGCCAACCACCGTCCCGCCGCCAATCGACCTCTCTAT | |
| | TPHGWTESHTTSG | CCCACACGTGTGGATGCCTCCACAAACCACACCACAC | |
| | LHRPHFNQTCILFD | GGATGGACTGAGTCCCATACCACCAGCGGACTGCACA | |
| | GHDLLFSTVTPCL | GGCCTCACTTCAATCAGACCTGCATTTTGTTCGACGGC | |
| | HQGFYLIDELRYV | CACGACCTCCTGTTCAGCACTGTGACCCCGTGTCTGCA | |
| | KITLTEDFFVVTVS | TCAGGGCTTCTACCTGATTGACGAGCTGAGGTACGTCA | |
| | IDDDTPMLLIFGHL | AGATTACGCTCACCGAAGACTTCTTCGTGGTCACAGTG | |
| | PRVLFKAPYQRDN | AGTATCGATGACGACACCCCTATGCTGCTCATCTTCGG | |
| | FILRQTEKHELLVL | CCATCTGCCTAGGGTGCTGTTCAAGGCCCCTTACCAGA | |
| | VKKDQLNRHSYL | GAGATAATTTCATCTTGCGGCAGACTGAGAAGCACGA | |
| | KDPDFLDAALDFN | ACTGCTGGTACTCGTGAAGAAGGACCAGCTGAACCGC | |
| | YLDLSALLRNSFH | CACTCTTACTTAAAGGATCCAGACTTCCTGGATGCAGC | |
| | RYAVDVLKSGRC | ACTTGACTTCAACTATCTCGACCTCTCTGCCCTGCTGA | |
| | QMLDRRTVEMAF | GGAACAGCTTCCACCGGTATGCCGTGGACGTGCTGAA | |
| | AYALALFAAARQ | GAGTGGACGGTGTCAGATGCTGGACCGCAGAACAGTG | |
| | EEAGAQVSVPRAL | GAAATGGCGTTCGCGTATGCTCTGGCCCTATTCGCGGC | |
| | DRQAALLQIQEFM | CGCAAGACAGGAGGAGGCCGGCGCTCAGGTGTCCGTC | |
| | ITCLSQTPPRTTLL | CCTAGAGCTCTGGACAGGCAGGCCGCCCTGCTGCAAA | |
| | LYPTAVDLAKRAL | TTCAGGAGTTCATGATAACTTGCCTGAGCCAAACCCCT | |
| | WTPNQITDITSLVR | CCGAGAACAACACTGCTGCTGTATCCAACAGCCGTAG | |
| | LVYILSKQNQQHL | ATCTGGCCAAGCGGGCCCTTTGGACTCCTAACCAGATC | |
| | IPQWALRQIADFA | ACCGATATTACCTCCCTGGTGAGACTGGTGTACATTCT | |
| | LKLHKTHLASFLS | GTCCAAGCAGAACCAGCAGCACCTGATCCCGCAGTGG | |
| | AFARQELYLMGSL | GCCCTGAGACAGATCGCTGATTTCGCCTTGAAGCTGCA | |
| | VHSMLVHTTERRE | CAAGACTCATCTGGCCTCCTTCCTGAGTGCTTTCGCCC | |
| | IFIVETGLCSLAEL | GCCAGGAACTGTATCTGATGGGCTCTCTTGTCCATTCC | |
| | SHFTQLLAHPHHE | ATGCTGGTTCATACCACGGAGAGAAGGGAGATCTTCA | |
| | YLSDLYTPCSSSG | TCGTGGAAACCGGCCTTTGCTCCCTCGCTGAGCTGAGC | |
| | RRDHSLERLTRLF | CATTTCACTCAGCTGCTCGCCCACCCGCACCACGAGTA | |
| | PDATVPATVPAAL | CCTGTCAGACCTTTATACTCCGTGCTCCTCCAGCGGCA | |
| | SILSTMQPSTLETF | GGAGGGACCACAGCCTGGAACGGCTCACAAGACTGTT | |
| | PDLFCLPLGESFSA | CCCGGATGCTACCGTGCCTGCTACTGTGCCAGCCGCCC | |
| | LTVSEHVSYIVTN | TGAGCATCCTTTCCACCATGCAGCCTTCCACACTGGAG | |
| | QYLIKGISYPVSTT | ACTTTCCCTGACCTGTTCTGCCTGCCACTTGGCGAAAG | |
| | VVGQSLIITQTDSQ | TTTCAGCGCCCTGACCGTGTCCGAACATGTGAGCTACA | |
| | TKCELTRNMHTTH | TCGTGACTAACCAGTACCTGATCAAGGGCATCAGCTA | |
| | SITVALNISLENCA | CCCGGTTAGCACCACTGTCGTCGGACAGTCACTGATCA | |
| | FCQSALLEYDDTQ | TCACTCAGACCGACTCCCAGACCAAGTGCGAACTGAC | |
| | GVINIMYMHDSDD | CAGAAATATGCACACAACCCATAGCATCACCGTGGCC | |
| | VLFALDPYNEVVV | CTGAACATTAGCCTGGAGAACTGTGCCTTCTGCCAGAG | |
| | SSPRTHYLMLLKN | CGCCCTCCTCGAGTACGACGATACCCAGGGTGTGATA | |
| | GTVLEVTDVVVD | AACATTATGTATATGCACGACAGTGACGACGTTCTGTT | |
| | ATDSRLLMMSVY | CGCACTGGACCCTTACAACGAAGTGGTCGTTTCCTCTC | |
| | ALSAIIGIYLLYRM | CTCGGACCCATTACCTGATGCTGCTGAAGAACGGCAC | |
| | LKTC (SEQ ID NO: | CGTGCTAGAGGTTACTGATGTGGTAGTGGACGCCACA | |
| | 59) | GACAGCAGACTGCTGATGATGAGCGTGTACGCCCTGA | |
| | | GCGCCATTATTGGCATCTACCTGCTGTACAGGATGCTG | |
| | | AAGACATGT (SEQ ID NO: 104) | |
| SE_CMV_gH_ | MRPGLPSYLIILAV | ATGCGGCCGGGACTCCCTTCCTACCTCATCATCCTTGC | C2/ |
| 033 | CLFSHLLSSRYGA | CGTGTGTCTTTTCTCACIACCTCCTCAGCAGCCGCTACG | CAP1/ |
| | EAVSEPLDKAFHL | GCGCCGAGGCCGTGTCAGAGCCACTTGACAAGGCCTT | T100 |
| | LLNTYGRPIRFLRE | CCATCTTTTGTTGAACACCTACGGCAGACCTATTCGGT | |
| | NTTQCTYNSSLRN | TCCTGAGAGAGAACACAACCCAGTGCACCTATAACAG | |
| | STVVRENAISFNFF | CTCTCTGCGCAACTCCACAGTGGTTAGAGAGAATGCC | |
| | QSYNQYYVFHMP | ATCAGCTTCAACTTCTTCCAGAGTTACAACCAGTATTA | |
| | RCLFAGPLAEQFL | CGTGTTCCATATGCCTAGGTGCCTGTTCGCTGGCCCGT | |
| | NQVDLTETLERYQ | TAGCCGAACAGTTCCTCAACCAGGTGGATCTGACCGA | |
| | QRLNTYALVSKDL | AACACTGGAAAGGTACCAGCAGCGGCTGAATACATAC | |
| | ASYRSFSQQLKAQ | GCCTTGGTGTCAAAGGATCTTGCTTCCTACAGAAGCTT | |
| | DSLGEQPTTVPPPI | CAGCCAGCAGCTGAAGGCCCAGGACAGCCTTGGAGAG | |
| | DLSIPHVWMPPQT | CAGCCAACCACCGTGCCTCCTCCTATTGACCTGAGCAT | |
| | TPHGWTESHTTSG | CCCTCATGTGTGGATGCCTCCTCAGACCACCCCTCACG | |
| | LHRPHFNQTCILFD | GCTGGACTGAGAGCCATACCACGTCCGGCCTGCACAG | |
| | GHDLLFSTVTPCL | GCCTCACTTCAATCAGACCTGCATCCTGTTCGACGGCC | |
| | HQGFYLIDELRYV | ACGATCTGCTTTTCAGCACCGTCACCCCTTGCCTGCAC | |
| | KITLTEDFFVVTVS | CAGGGATTCTACCTGATCGACGAGCTCCGGTATGTGA | |
| | IDDDTPMLLIFGHL | AGATTACACTGACCGAGGACTTCTTCGTGGTGACCGTG | |
| | PRVLFKAPYQRDN | TCCATCGACGATGACACCCCAATGCTGCTGATCTTCGG | |
| | FILRQTEKHELLVL | CCACCTGCCGAGAGTCCTGTTCAAGGCCCCATACCAG | |
| | VKKDQLNRHSYL | AGAGACAACTTCATCCTGCGGCAGACCGAGAAGCACG | |
| | KDPDFLDAALDFN | AACTGCTAGTGCTGGTGAAGAAGGATCAGCTGAACCG | |
| | YLDLSALLRNSFH | GCACTCCTACCTGAAGGACCCTGACTTCCTTGACGCCG | |
| | RYAVDVLKSGRC | CACTCGACTTCAACTACCTGGACCTCAGTGCTCTACTG | |
| | QMLDRRTVEMAF | AGGAACTCTTTCCACCGGTACGCCGTGGACGTGCTGA | |
| | AYALALFAAARQ | AGTCTGGAAGATGCCAGATGCTGGATAGGAGGACAGT | |
| | EEAGAQVSVPRAL | GGAGATGGCGTTCGCGTACGCCCTGGCCCTGTTCGCCG | |
| | DRQAALLQIQEFM | CCGCCAGACAGGAGGAGGCCGGCGCACAGGTCAGCGT | |
| | ITCLSQTPPRTTLL | CCCAAGGGCCCTGGACCGCCAGGCTGCCCTGCTGCAG | |
| | LYPTAVDLAKRAL | ATTCAGGAATTCATGATCACCTGTCTCAGCCAGACCCC | |
| | WTPNQITDITSLVR | TCCGAGAACAACCCTGCTGTTGTACCCGACCGCAGTG | |
| | LVYILSKQNQQHL | GATCTGGCTAAGAGGGCCCTGTGGACCCCAAACCAGA | |
| | IPQWALRQIADFA | TTACCGACATCACCTCTCTGGTGAGACTGGTGTACATC | |
| | LKLHKTHLASFLS | CTGTCCAAGCAGAACCAACAGCACCTCATTCCACAAT | |
| | AFARQELYLMGSL | GGGCCCTGAGGCAAATCGCCGATTTCGCTCTCAAGTTG | |
| | VHSMLVHTTERRE | CATAAGACCCATCTGGCCTCATTCCTCAGCGCCTTCGC | |
| | IFIVETGLCSLAEL | AAGACAGGAGTTGTATCTCATGGGCTCCCTCGTGCATA | |
| | SHFTQLLAHPHHE | GCATGCTGGTGCACACAACCGAGCGCAGAGAAATTTT | |
| | YLSDLYTPCSSSG | CATCGTTGAAACCGGACTGTGCAGCCTCGCCGAGTTGT | |
| | RRDHSLERLTRLF | CTCATTTCACCCAGCTGCTGGCTCATCCTCACCATGAG | |
| | PDATVPATVPAAL | TATCTTTCCGACCTGTACACCCCGTGCAGCAGCAGCGG | |
| | SILSTMQPSTLETF | CCGCAGGGATCACAGCCTCGAGAGACTGACAAGACTG | |
| | PDLFCLPLGESFSA | TTCCCAGACGCCACCGTGCCTGCCACAGTGCCAGCCGC | |
| | LTVSEHVSYIVTN | GCTGTCCATCCTGAGCACAATGCAGCCTAGCACACTG | |
| | QYLIKGISYPVSTT | GAGACTTTCCCAGATCTGTTCTGTCTTCCACTGGGCGA | |
| | VVGQSLIITQTDSQ | GAGCTTCAGCGCCCTGACCGTGAGCGAGCACGTGAGC | |
| | TKCELTRNMHTTH | TACATAGTGACCAACCAATATTTGATTAAGGGCATCTC | |
| | SITVALNISLENCA | CTACCCTGTGAGCACCACAGTGGTGGGCCAGTCTCTGA | |
| | FCQSALLEYDDTQ | TCATCACACAAACCGACAGTCAGACGAAGTGCGAGCT | |
| | GVINIMYMHDSDD | GACTAGAAACATGCACACGACCCACAGCATAACCGTG | |
| | VLFALDPYNEVVV | GCACTCAACATCTCCCTGGAGAATTGCGCCTTCTGCCA | |
| | SSPRTHYLMLLKN | GAGCGCCCTCCTGGAGTACGACGACACTCAAGGAGTG | |
| | GTVLEVTDVVVD | ATCAACATCATGTACATGCACGATAGCGATGACGTGC | |
| | ATDSRLLMMSVY | TGTTCGCCCTGGACCCATACAATGAAGTGGTGGTGTCC | |
| | ALSAIIGIYLLYRM | AGCCCACGGACCCACTACCTGATGCTCCTCAAGAACG | |
| | LKTC (SEQ ID NO: | GCACAGTGCTGGAGGTTACAGACGTGGTGGTCGACGC | |
| | 59) | TACCGATAGCAGACTTCTTATGATGTCCGTGTACGCCC | |
| | | TGAGCGCCATCATCGGAATCTATCTGCTTTACAGGATG | |
| | | CTGAAGACTTGC (SEQ ID NO: 105) | |
| SE_CMV_gH_ | MRPGLPSYLIILAV | ATGCGGCCAGGCCTCCCTTCGTACCTTATCATCTTGGC | C2/ |
| 034 | CLFSHLLSSRYGA | CGTGTGTCTTTTCTCTCACCTACTCTCCTCAAGGTACGG | CAP1/ |
| | EAVSEPLDKAFHL | CGCCGAGGCCGTGAGTGAGCCGCTAGACAAGGCCTTC | T100 |
| | LLNTYGRPIRFLRE | CACCTATTACTTAACACCTACGGCCGGCCTATCCGATT | |
| | NTTQCTYNSSLRN | CCTCCGGGAGAATACCACACAGTGTACATACAACTCT | |
| | STVVRENAISFNFF | AGCCTGCGCAACAGCACTGTGGTCAGGGAGAACGCCA | |
| | QSYNQYYVFHMP | TCAGCTTCAATTTCTTCCAGAGTTATAACCAGTACTAC | |
| | RCLFAGPLAEQFL | GTGTTCCACATGCCAAGATGCCTGTTCGCCGGACCTCT | |
| | NQVDLTETLERYQ | TGCCGAGCAGTTCCTGAACCAGGTGGATCTGACCGAG | |
| | QRLNTYALVSKDL | ACACTGGAGAGATACCAGCAAAGGCTCAATACCTACG | |
| | ASYRSFSQQLKAQ | CTTTAGTGAGCAAGGACCTTGCTTCTTACAGATCTTTC | |
| | DSLGEQPTTVPPPI | TCACAACAGCTTAAGGCGCAGGACAGCCTCGGCGAGC | |
| | DLSIPHVWMPPQT | AGCCTACCACCGTGCCTCCTCCTATTGACTTGAGCATC | |
| | TPHGWTESHTTSG | CCACACGTATGGATGCCTCCACAGACGACACCACACG | |
| | LHRPHFNQTCILFD | GCTGGACCGAATCCCATACAACCAGCGGACTCCACCG | |
| | GHDLLFSTVTPCL | GCCTCATTTCAATCAGACCTGCATCCTTTTCGACGGCC | |
| | HQGFYLIDELRYV | ATGACCTGCTATTCTCTACCGTCACCCCGTGCCTGCAC | |
| | KITLTEDFFVVTVS | CAGGGCTTCTACCTTATCGACGAACTGAGATACGTCAA | |
| | IDDDTPMLLIFGHL | GATCACGCTGACCGAAGACTTCTTCGTCGTTACAGTCA | |
| | PRVLFKAPYQRDN | GCATCGACGACGATACCCCTATGCTGCTGATATTCGGC | |
| | FILRQTEKHELLVL | CACCTTCCTAGAGTCCTGTTCAAGGCACCGTACCAGAG | |
| | VKKDQLNRHSYL | GGACAACTTCATCCTGAGACAGACAGAGAAGCACGAG | |
| | KDPDFLDAALDFN | CTCCTGGTGCTGGTGAAGAAGGATCAGCTGAATAGAC | |
| | YLDLSALLRNSFH | ACAGTTACCTGAAGGATCCAGACTTCCTGGACGCCGC | |
| | RYAVDVLKSGRC | ACTGGACTTCAACTATCTGGATCTGAGCGCCCTGCTTC | |
| | QMLDRRTVEMAF | GCAATAGCTTCCATAGATACGCTGTGGACGTGCTGAA | |
| | AYALALFAAARQ | GTCTGGCCGGTGTCAGATGCTGGATCGTAGGACCGTG | |
| | EEAGAQVSVPRAL | GAGATGGCCTTCGCCTACGCACTCGCTCTGTTCGCCGC | |
| | DRQAALLQIQEFM | CGCTAGACAGGAGGAGGCCGGAGCCCAAGTGAGTGTG | |
| | ITCLSQTPPRTTLL | CCTCGGGCACTGGACAGACAGGCAGCCTTACTGCAGA | |
| | LYPTAVDLAKRAL | TCCAGGAGTTCATGATTACCTGCCTGTCTCAGACTCCA | |
| | WTPNQITDITSLVR | CCACGGACCACCCTTCTGCTGTATCCTACCGCGGTTGA | |
| | LVYILSKQNQQHL | TCTGGCTAAGAGGGCCCTGTGGACCCCTAACCAGATC | |
| | IPQWALRQIADFA | ACTGACATCACCAGCCTCGTGAGGCTGGTGTACATTCT | |
| | LKLHKTHLASFLS | TAGCAAGCAGAACCAGCAGCACCTAATACCTCAGTGG | |
| | AFARQELYLMGSL | GCCCTGCGGCAGATCGCCGACTTCGCCCTGAAGCTGC | |
| | VHSMLVHTTERRE | ACAAGACCCACCTGGCAAGCTTCCTGTCCGCCTTCGCC | |
| | IFIVETGLCSLAEL | CGCCAGGAGCTGTACCTCATGGGAAGTCTGGTACACT | |
| | SHFTQLLAHPHHE | CCATGCTGGTGCACACCACCGAGAGAAGAGAGATCTT | |
| | YLSDLYTPCSSSG | CATCGTAGAAACCGGACTCTGCTCACTGGCCGAATTGT | |
| | RRDHSLERLTRLF | CACACTTCACCCAGCTGCTGGCCCATCCTCATCACGAG | |
| | PDATVPATVPAAL | TATCTGTCCGACCTGTACACCCCTTGCAGCTCTAGCGG | |
| | SILSTMQPSTLETF | CAGGCGGGACCATTCCTTGGAGAGGCTGACCAGGCTG | |
| | PDLFCLPLGESFSA | TTCCCGGATGCCACCGTTCCAGCAACAGTGCCTGCAGC | |
| | LTVSEHVSYIVTN | CCTGAGCATTCTGTCAACAATGCAGCCTAGCACCCTCG | |
| | QYLIKGISYPVSTT | AAACTTTCCCGGATCTGTTCTGCCTGCCTCTTGGTGAG | |
| | VVGQSLIITQTDSQ | AGCTTCAGCGCCCTGACCGTGTCCGAGCATGTGTCTTA | |
| | TKCELTRNMHTTH | TATCGTAACCAATCAGTACCTGATCAAGGGCATCAGCT | |
| | SITVALNISLENCA | ACCCTGTGTCCACAACTGTCGTGGGCCAAAGCCTCATC | |
| | FCQSALLEYDDTQ | ATAACCCAGACCGATTCCCAGACAAAGTGTGAACTGA | |
| | GVINIMYMHDSDD | CCCGCAACATGCACACCACTCACAGCATTACTGTGGCC | |
| | VLFALDPYNEVVV | CTGAACATCTCCCTGGAGAACTGTGCCTTCTGTCAGAG | |
| | SSPRTHYLMLLKN | CGCGCTTCTGGAGTATGATGACACCCAGGGTGTGATTA | |
| | GTVLEVTDVVVD | ATATCATGTACATGCACGACAGCGACGATGTGCTGTTC | |
| | ATDSRLLMMSVY | GCGCTGGATCCTTACAATGAGGTGGTCGTGAGCTCCCC | |
| | ALSAIIGIYLLYRM | TAGAACCCACTATCTCATGCTGTTAAAGAACGGCACC | |
| | LKTC (SEQ ID NO: | GTCCTGGAGGTGACAGACGTGGTGGTTGATGCCACCG | |
| | 59) | ACAGCAGGCTGCTGATGATGAGCGTTTATGCCCTGAG | |
| | | CGCCATCATCGGCATTTACCTCCTGTACAGGATGTTAA | |
| | | AGACTTGT (SEQ ID NO: 106) | |
| SE_CMV_gH_ | MRPGLPSYLIILAV | ATGCGGCCAGGACTCCCTAGCTACCTCATTATCCTCGC | C2/ |
| 035 | CLFSHLLSSRYGA | CGTGTGCCTTTTCTCTCACCTCTTAAGCTCCCGGTACG | CAP1/ |
| | EAVSEPLDKAFHL | GCGCCGAAGCCGTGAGCGAGCCGCTTGACAAGGCTTT | T100 |
| | LLNTYGRPIRFLRE | CCACCTCCTCCTTAACACCTACGGCAGACCTATTAGAT | |
| | NTTQCTYNSSLRN | TCCTGAGAGAGAACACCACCCAGTGTACATATAATTCT | |
| | STVVRENAISFNFF | AGCCTGCGGAACTCCACCGTAGTGAGGGAGAATGCCA | |
| | QSYNQYYVFHMP | TTAGCTTCAACTTCTTCCAGAGCTACAACCAGTACTAT | |
| | RCLFAGPLAEQFL | GTGTTCCATATGCCGAGATGTCTGTTCGCTGGACCTCT | |
| | NQVDLTETLERYQ | CGCAGAACAGTTCTTGAACCAGGTGGATCTGACTGAA | |
| | QRLNTYALVSKDL | ACTCTCGAGCGGTACCAGCAAAGACTGAATACCTATG | |
| | ASYRSFSQQLKAQ | CCTTGGTAAGCAAGGATCTGGCTAGCTACAGGAGCTT | |
| | DSLGEQPTTVPPPI | CTCCCAGCAGCTCAAGGCCCAGGACTCCCTTGGCGAA | |
| | DLSIPHVWMPPQT | CAGCCTACCACCGTCCCTCCTCCAATTGACCTGAGCAT | |
| | TPHGWTESHTTSG | TCCGCACGTGTGGATGCCTCCTCAGACCACCCCACACG | |
| | LHRPHFNQTCILFD | GCTGGACAGAGTCTCATACCACCAGCGGACTGCATAG | |
| | GHDLLFSTVTPCL | ACCGCATTTCAACCAGACTTGCATCCTGTTCGATGGAC | |
| | HQGFYLIDELRYV | ATGATCTCCTGTTCTCTACAGTGACTCCATGCCTGCAC | |
| | KITLTEDFFVVTVS | CAGGGCTTCTACCTGATCGATGAGCTCAGATACGTCAA | |
| | IDDDTPMLLIFGHL | GATCACCTTGACCGAAGATTTCTTCGTGGTCACAGTGA | |
| | PRVLFKAPYQRDN | GCATTGACGACGACACCCCAATGCTTCTGATATTCGGT | |
| | FILRQTEKHELLVL | CACCTGCCTAGGGTCCTCTTCAAGGCTCCATACCAGAG | |
| | VKKDQLNRHSYL | AGACAATTTCATCCTTAGACAGACCGAGAAGCACGAG | |
| | KDPDFLDAALDFN | CTGCTCGTGCTGGTGAAGAAGGATCAACTGAACAGAC | |
| | YLDLSALLRNSFH | ATAGCTACCTAAAGGATCCGGATTTCCTGGACGCCGCT | |
| | RYAVDVLKSGRC | CTGGACTTCAACTACCTCGACCTCAGCGCCCTGCTGAG | |
| | QMLDRRTVEMAF | GAACAGCTTCCACCGGTATGCAGTCGATGTTCTCAAGT | |
| | AYALALFAAARQ | CCGGCAGATGCCAGATGCTGGACCGTAGAACTGTGGA | |
| | EEAGAQVSVPRAL | GATGGCCTTCGCCTATGCTCTGGCCCTGTTCGCCGCCG | |
| | DRQAALLQIQEFM | CACGCCAGGAAGAGGCTGGAGCCCAGGTGAGCGTCCC | |
| | ITCLSQTPPRTTLL | ACGGGCTCTGGACAGACAGGCTGCTCTGCTGCAGATC | |
| | LYPTAVDLAKRAL | CAAGAGTTCATGATTACCTGTCTGAGCCAGACCCCTCC | |
| | WTPNQITDITSLVR | TAGAACCACCCTCCTCCTCTATCCGACCGCTGTGGACC | |
| | LVYILSKQNQQHL | TGGCCAAGAGAGCCTTGTGGACCCCTAATCAGATTACT | |
| | IPQWALRQIADFA | GACATCACAAGCCTGGTCAGACTGGTGTATATCCTGA | |
| | LKLHKTHLASFLS | GCAAGCAGAATCAGCAGCACCTCATTCCACAGTGGGC | |
| | AFARQELYLMGSL | GCTGCGGCAGATCGCTGATTTCGCCCTGAAGCTGCACA | |
| | VHSMLVHTTERRE | AGACCCACCTGGCCAGCTTCTTGAGCGCATTCGCACGG | |
| | IFIVETGLCSLAEL | CAGGAACTCTACCTGATGGGCTCTCTGGTGCACAGCAT | |
| | SHFTQLLAHPHHE | GCTCGTCCACACCACAGAACGGCGAGAGATATTCATC | |
| | YLSDLYTPCSSSG | GTTGAGACAGGCCTGTGCTCTCTGGCCGAGTTGTCCCA | |
| | RRDHSLERLTRLF | CTTCACCCAACTGCTGGCTCACCCTCATCACGAGTACC | |
| | PDATVPATVPAAL | TCAGCGACCTGTACACCCCTTGCTCCTCCAGCGGTAGA | |
| | SILSTMQPSTLETF | CGGGATCACAGCCTGGAAAGACTGACCAGACTGTTCC | |
| | PDLFCLPLGESFSA | CAGACGCCACGGTCCCTGCAACCGTGCCTGCCGCTCTT | |
| | LTVSEHVSYIVTN | TCAATCTTGTCCACCATGCAGCCTAGTACACTGGAAAC | |
| | QYLIKGISYPVSTT | ATTCCCTGACCTCTTCTGCCTGCCTCTCGGAGAGTCCTT | |
| | VVGQSLIITQTDSQ | CTCAGCCCTGACCGTGAGCGAACACGTGTCCTACATCG | |
| | TKCELTRNMHTTH | TGACCAACCAGTACCTGATCAAGGGCATCTCCTACCCT | |
| | SITVALNISLENCA | GTGTCGACCACCGTCGTGGGCCAGAGCCTGATCATTAC | |
| | FCQSALLEYDDTQ | ACAGACGGACTCTCAGACCAAGTGCGAGTTGACACGG | |
| | GVINIMYMHDSDD | AACATGCACACCACACACAGCATCACTGTGGCCCTGA | |
| | VLFALDPYNEVVV | ATATTAGCCTCGAGAACTGCGCCTTCTGCCAGAGTGCC | |
| | SSPRTHYLMLLKN | CTGCTAGAGTATGATGATACACAGGGCGTGATTAATA | |
| | GTVLEVTDVVVD | TCATGTATATGCACGACTCTGATGACGTCCTGTTCGCC | |
| | ATDSRLLMMSVY | CTGGACCCATACAACGAGGTGGTTGTGAGCTCCCCTCG | |
| | ALSAIIGIYLLYRM | GACCCACTATCTGATGCTGCTCAAGAACGGCACTGTTC | |
| | LKTC (SEQ ID NO: | TCGAGGTGACAGATGTGGTGGTCGATGCCACAGATTC | |
| | 59) | TCGGCTGCTGATGATGAGCGTGTACGCTCTTAGCGCCA | |
| | | TCATCGGAATCTACCTCCTGTACAGGATGCTGAAGACT | |
| | | TGT (SEQ ID NO: 107) | |
| SE_CMV_gH_ | MRPGLPSYLIILAV | ATGCGGCCGGGCCTCCCGTCTTACCTCATCATCTTGGC | C2/ |
| 036 | CLFSHLLSSRYGA | CGTGTGCCTCTTCTCCCACCTCTTGAGCTCCCGGTACG | CAP1/ |
| | EAVSEPLDKAFHL | GCGCAGAGGCCGTGTCAGAGCCTCTCGACAAGGCCTT | T100 |
| | LLNTYGRPIRFLRE | CCATTTGCTCCTTAACACATACGGCAGGCCAATCAGGT | |
| | NTTQCTYNSSLRN | TCCTGCGAGAGAATACCACACAATGCACCTACAACTC | |
| | STVVRENAISFNFF | CAGCTTGAGGAATAGCACCGTGGTGCGGGAGAACGCC | |
| | QSYNQYYVFHMP | ATCTCCTTCAATTTCTTCCAGTCCTACAATCAGTATTAT | |
| | RCLFAGPLAEQFL | GTGTTCCATATGCCTAGGTGTCTCTTCGCAGGCCCACT | |
| | NQVDLTETLERYQ | TGCCGAACAATTCCTGAACCAAGTGGACCTGACAGAG | |
| | QRLNTYALVSKDL | ACACTGGAGAGATACCAGCAACGGCTGAATACCTACG | |
| | ASYRSFSQQLKAQ | CCTTGGTGAGCAAGGATCTCGCCAGCTACAGATCTTTC | |
| | DSLGEQPTTVPPPI | TCACAACAACTGAAGGCCCAGGATTCTCTCGGTGAGC | |
| | DLSIPHVWMPPQT | AGCCAACGACTGTGCCTCCTCCAATTGACCTGTCTATC | |
| | TPHGWTESHTTSG | CCACATGTGTGGATGCCACCTCAGACTACCCCTCACGG | |
| | LHRPHFNQTCILFD | ATGGACAGAGTCTCATACCACTAGCGGCCTGCACAGG | |
| | GHDLLFSTVTPCL | CCTCACTTCAATCAGACCTGTATCCTCTTCGACGGTCA | |
| | HQGFYLIDELRYV | CGATCTGTTGTTCAGCACCGTGACCCCATGCCTGCATC | |
| | KITLTEDFFVVTVS | AGGGCTTCTACCTGATTGACGAGCTGAGATATGTGAA | |
| | IDDDTPMLLIFGHL | GATAACACTGACCGAGGATTTCTTCGTGGTCACCGTGA | |
| | PRVLFKAPYQRDN | GCATAGACGACGACACACCGATGCTCCTGATCTTCGG | |
| | FILRQTEKHELLVL | CCATCTGCCACGAGTTCTGTTCAAGGCACCTTATCAGA | |
| | VKKDQLNRHSYL | GAGACAACTTCATCTTGAGGCAAACAGAGAAGCACGA | |
| | KDPDFLDAALDFN | GCTTCTCGTGCTGGTTAAGAAGGACCAGCTCAACAGG | |
| | YLDLSALLRNSFH | CATAGCTACCTGAAGGACCCAGATTTCCTGGACGCCG | |
| | RYAVDVLKSGRC | CTCTGGATTTCAATTATCTGGACCTTTCTGCTCTGCTGA | |
| | QMLDRRTVEMAF | GAAACAGCTTCCATAGATACGCCGTGGACGTCCTTAA | |
| | AYALALFAAARQ | GTCTGGCCGCTGCCAGATGCTGGATAGACGGACTGTC | |
| | EEAGAQVSVPRAL | GAGATGGCATTCGCCTACGCTCTGGCTCTGTTCGCCGC | |
| | DRQAALLQIQEFM | CGCCAGGCAGGAGGAGGCTGGAGCCCAAGTGTCAGTG | |
| | ITCLSQTPPRTTLL | CCTAGGGCTCTGGATAGACAAGCCGCCTTGCTCCAGAT | |
| | LYPTAVDLAKRAL | CCAGGAGTTCATGATTACCTGTCTGAGCCAGACCCCAC | |
| | WTPNQITDITSLVR | CAAGAACCACGTTACTGCTGTACCCTACCGCTGTGGAC | |
| | LVYILSKQNQQHL | CTGGCTAAGCGAGCCCTCTGGACGCCTAATCAAATCA | |
| | IPQWALRQIADFA | CCGACATCACCAGCTTAGTCAGACTGGTGTACATTCTG | |
| | LKLHKTHLASFLS | TCTAAGCAGAACCAGCAGCACTTGATTCCACAGTGGG | |
| | AFARQELYLMGSL | CCCTGAGACAGATTGCCGACTTCGCCCTGAAGCTCCAT | |
| | VHSMLVHTTERRE | AAGACCCATCTGGCGTCCTTCCTGAGCGCCTTCGCCAG | |
| | IFIVETGLCSLAEL | ACAGGAGCTCTACCTGATGGGCAGCCTGGTTCATTCCA | |
| | SHFTQLLAHPHHE | TGCTGGTCCATACAACGGAGAGAAGAGAGATCTTCAT | |
| | YLSDLYTPCSSSG | CGTGGAGACAGGACTGTGCTCTTTGGCCGAACTTTCCC | |
| | RRDHSLERLTRLF | ACTTCACTCAGCTGCTGGCGCACCCTCATCACGAGTAC | |
| | PDATVPATVPAAL | TTATCGGACCTGTACACCCCTTGCAGCAGCAGCGGAA | |
| | SILSTMQPSTLETF | GGAGGGACCATTCTCTCGAAAGGCTGACAAGACTGTT | |
| | PDLFCLPLGESFSA | CCCTGACGCCACCGTCCCAGCCACAGTGCCTGCCGCAC | |
| | LTVSEHVSYIVTN | TGAGCATCCTCAGCACAATGCAGCCAAGCACTCTGGA | |
| | QYLIKGISYPVSTT | GACTTTCCCGGACTTGTTCTGCCTGCCGCTGGGCGAGT | |
| | VVGQSLIITQTDSQ | CCTTCAGCGCCCTTACAGTGTCAGAGCATGTGTCCTAC | |
| | TKCELTRNMHTTH | ATCGTGACCAATCAGTACCTGATCAAGGGAATCAGCT | |
| | SITVALNISLENCA | ACCCTGTGTCTACAACCGTGGTTGGGCCAGTCCCTCATC | |
| | PCQSALLEYDDTQ | ATCACCCAGACAGATAGCCAAACTAAGTGCGAACTGA | |
| | GVINIMYMHDSDD | CTAGAAACATGCACACAACCCACTCCATCACAGTGGC | |
| | VLFALDPYNEVVV | CCTGAACATCAGCCTCGAGAATTGCGCCTTCTGCCAGA | |
| | SSPRTHYLMLLKN | GCGCACTGTTGGAGTACGACGATACTCAGGGCGTGAT | |
| | GTVLEVTDVVVD | TAACATCATGTACATGCATGATAGCGACGATGTGCTGT | |
| | ATDSRLLMMSVY | TCGCCCTGGACCCTTATAACGAGGTGGTGGTGAGTAGT | |
| | ALSAIIGIYLLYRM | CCTAGGACCCATTACCTTATGCTGCTGAAGAACGGAA | |
| | LKTC (SEQ ID NO: | CTGTTCTGGAGGTTACCGACGTCGTCGTTGACGCTACC | |
| | 59) | GACTCACGCCTGCTCATGATGTCTGTGTATGCCCTGTC | |
| | | TGCCATCATCGGCATCTACCTGCTGTATAGGATGCTGA | |
| | | AGACTTGC (SEQ ID NO: 108) | |
| SE_CMV_gH_ | MRPGLPSYLIILAV | ATGCGGCCCGGCCTTCCCAGCTACCTCATCATCCTCGC | C2/ |
| 037 | CLFSHLLSSRYGA | CGTGTGCTTGTTCAGCCACCTTCTAAGCAGCCGGTACG | CAP1/ |
| | EAVSEPLDKAFHL | GCGCCGAGGCCGTGAGCGAGCCCCTCGACAAGGCCTT | T100 |
| | LLNTYGRPIRFLRE | CCACTTGTTATTGAACACCTACGGCCGGCCCATCCGGT | |
| | NTTQCTYNSSLRN | TCCTGCGGGAGAACACCACCCAGTGCACCTACAACAG | |
| | STVVRENAISFNFF | CAGCCTGCGGAACAGCACCGTGGTTCGGGAGAATGCC | |
| | QSYNQYYVFHMP | ATCAGCTTCAACTTCTTCCAGAGCTACAACCAGTACTA | |
| | RCLFAGPLAEQFL | CGTGTTCCACATGCCCCGGTGTCTTTTCGCCGGACCGC | |
| | NQVDLTETLERYQ | TGGCCGAGCAGTTCCTGAACCAGGTGGACCTGACCGA | |
| | QRLNTYALVSKDL | AACTCTGGAGCGGTACCAGCAGCGGCTGAATACCTAT | |
| | ASYRSFSQQLKAQ | GCCCTGGTGAGCAAGGACCTGGCCTCATACCGGAGCT | |
| | DSLGEQPTTVPPPI | TCAGCCAGCAGCTGAAGGCCCAGGACAGCCTGGGCGA | |
| | DLSIPHVWMPPQT | GCAGCCCACCACCGTGCCTCCACCCATCGACCTGAGC | |
| | TPHGWTESHTTSG | ATCCCGCACGTGTGGATGCCTCCACAGACCACACCTCA | |
| | LHRPHFNQTCILFD | CGGCTGGACCGAGAGCCACACCACCAGCGGCCTGCAC | |
| | GHDLLFSTVTPCL | CGGCCCCACTTCAACCAGACCTGCATCCTGTTCGACGG | |
| | HQGFYLIDELRYV | CCACGACCTGCTGTTCAGCACAGTTACCCCTTGCCTTC | |
| | KITLTEDFFVVTVS | ATCAGGGCTTCTATCTTATAGACGAGCTGCGGTACGTG | |
| | IDDDTPMLLIFGHL | AAGATCACACTCACCGAGGACTTCTTCGTGGTGACCGT | |
| | PRVLFKAPYQRDN | GAGCATCGACGACGACACTCCTATGCTCCTCATCTTCG | |
| | FILRQTEKHELLVL | GCCATCTTCCCCGGGTTCTGTTCAAGGCTCCATACCAG | |
| | VKKDQLNRHSYL | CGGGACAACTTCATCCTGCGGCAGACCGAGAAGCACG | |
| | KDPDFLDAALDFN | AGTTGCTGGTGCTGGTGAAGAAGGACCAGCTGAACCG | |
| | YLDLSALLRNSFH | GCATTCGTACCTTAAGGACCCCGACTTCCTGGACGCCG | |
| | RYAVDVLKSGRC | CCCTGGACTTCAACTACCTAGATCTCAGCGCCCTGTTG | |
| | QMLDRRTVEMAF | AGGAATAGCTTCCACCGGTACGCCGTGGACGTGTTAA | |
| | AYALALFAAARQ | AGAGCGGCCGGTGCCAGATGCTGGATCGGCGGACCGT | |
| | EEAGAQVSVPRAL | GGAGATGGCCTTCGCCTACGCGCTGGCCTTGTTCGCTG | |
| | DRQAALLQIQEFM | CCGCCCGGCAGGAGGAGGCCGGCGCCCAGGTGAGCGT | |
| | ITCLSQTPPRTTLL | ACCGCGGGCACTCGATCGGCAGGCCGCTCTGCTGCAG | |
| | LYPTAVDLAKRAL | ATCCAGGAGTTCATGATCACCTGCCTGAGCCAGACCCC | |
| | WTPNQITDITSLVR | TCCGCGGACCACCTTACTGCTTTACCCCACTGCAGTTG | |
| | LVYILSKQNQQHL | ACCTGGCTAAGCGCGCACTCTGGACCCCTAACCAGAT | |
| | IPQWALRQIADFA | CACCGACATCACCAGCCTGGTGCGGCTGGTGTACATCC | |
| | LKLHKTHLASFLS | TGAGCAAGCAGAACCAGCAACACCTGATACCACAGTG | |
| | AFARQELYLMGSL | GGCTCTGAGACAGATCGCCGACTTCGCCCTGAAGCTG | |
| | VHSMLVHTTERRE | CACAAGACCCATCTGGCCAGCTTCCTGTCCGCTTTCGC | |
| | IFIVETGLCSLAEL | ACGACAGGAGCTGTACCTGATGGGATCACTCGTGCAC | |
| | SHFTQLLAHPHHE | AGCATGCTCGTGCATACCACCGAGCGGCGGGAGATCT | |
| | YLSDLYTPCSSSG | TCATCGTGGAAACAGGCCTGTGTTCACTAGCCGAACTG | |
| | RRDHSLERLTRLF | AGCCACTTCACCCAACTTTTGGCCCATCCACACCACGA | |
| | PDATVPATVPAAL | GTATTTGTCGGACCTGTACACCCCTTGTTCCTCTTCCGG | |
| | SILSTMQPSTLETF | AAGGCGGGACCACTCCCTGGAACGGCTGACCCGGCTG | |
| | PDLFCLPLGESFSA | TTCCCCGACGCAACCGTACCGGCCACGGTTCCAGCTGC | |
| | LTVSEHVSYIVTN | CTTAAGCATCTTAAGTACCATGCAGCCCAGCACACTGG | |
| | QYLIKGISYPVSTT | AAACCTTCCCAGATCTGTTCTGCCTGCCGCTGGGTGAG | |
| | VVGQSLIITQTDSQ | TCTTTCAGCGCTCTCACCGTGTCCGAGCACGTGAGCTA | |
| | TKCELTRNMHTTH | CATCGTGACAAATCAATATCTGATTAAGGGCATCAGCT | |
| | SITVALNISLENCA | ACCCAGTGTCAACTACGGTGGTTGGCCAGAGCTTGATT | |
| | PCQSALLEYDDTQ | ATAACCCAGACCGACTCGCAGACTAAGTGCGAGCTTA | |
| | GVINIMYMHDSDD | CGAGAAACATGCACACAACCCACAGCATCACCGTGGC | |
| | VLFALDPYNEVVV | CCTGAACATAAGTCTGGAGAACTGCGCCTTCTGCCAGT | |
| | SSPRTHYLMLLKN | CTGCCTTGCTCGAGTATGATGACACCCAGGGCGTGATC | |
| | GTVLEVTDVVVD | AACATCATGTACATGCATGACAGCGACGATGTTCTCTT | |
| | ATDSRLLMMSVY | CGCGTTGGATCCATACAACGAGGTGGTGGTGTCCAGT | |
| | ALSAIIGIYLLYRM | CCGAGAACTCACTATCTGATGCTCCTAAAGAACGGCA | |
| | LKTC (SEQ ID NO: | CCGTGCTGGAGGTGACCGACGTCGTGGTCGATGCCAC | |
| | 59) | GGACTCCAGACTGCTTATGATGAGCGTGTACGCCCTAA | |
| | | GCGCCATCATCGGCATCTATCTCCTGTATCGGATGCTT | |
| | | AAGACCTGC (SEQ ID NO: 109) | |
| SE_CMV_gH_ | MRPGLPSYLIILAV | ATGCGGCCCGGCCTTCCCAGCTACCTCATCATCCTCGC | C2/ |
| 038 | CLFSHLLSSRYGA | CGTGTGCTTGTTCAGCCACTTGCTTAGCAGCCGGTACG | CAP1/ |
| | EAVSEPLDKAFHL | GCGCCGAGGCCGTGAGCGAGCCCTTGGACAAGGCCTT | T100 |
| | LLNTYGRPIRFLRE | CCACTTACTCCTCAACACCTACGGCCGGCCCATCCGGT | |
| | NTTQCTYNSSLRN | TCCTGCGGGAGAACACCACCCAGTGCACCTACAACAG | |
| | STVVRENAISFNFF | CAGCCTGCGGAACAGCACCGTGGTGCGTGAGAACGCC | |
| | QSYNQYYVFHMP | ATCAGCTTCAACTTCTTCCAGAGCTACAACCAGTACTA | |
| | RCLFAGPLAEQFL | CGTGTTCCACATGCCCAGGTGCCTTTTCGCCGGACCAC | |
| | NQVDLTETLERYQ | TGGCCGAGCAGTTCCTGAACCAGGTGGACCTGACCGA | |
| | QRLNTYALVSKDL | AACACTGGAGCGGTACCAGCAGAGGCTGAACACATAC | |
| | ASYRSFSQQLKAQ | GCCCTGGTGAGCAAGGACCTGGCCTCCTACCGGAGCT | |
| | DSLGEQPTTVPPPI | TCAGCCAGCAGCTGAAGGCCCAGGACAGCCTGGGCGA | |
| | DLSIPHVWMPPQT | GCAGCCCACCACCGTGCCGCCACCCATCGACCTGAGC | |
| | TPHGWTESHTTSG | ATCCCACACGTGTGGATGCCACCACAGACCACCCCTC | |
| | LHRPHFNQTCILFD | ACGGCTGGACCGAGAGCCACACCACCAGCGGCCTGCA | |
| | GHDLLFSTVTPCL | CCGGCCCCACTTCAACCAGACCTGCATCCTGTTCGACG | |
| | HQGFYLIDELRYV | GCCACGACCTGCTGTTCTCGACCGTCACCCCTTGCTTG | |
| | KITLTEDFFVVTVS | CACCAGGGCTTCTATCTGATAGACGAGCTGCGGTACGT | |
| | IDDDTPMLLIFGHL | GAAGATCACCTTGACCGAGGACTTCTTCGTGGTGACCG | |
| | PRVLFKAPYQRDN | TGAGCATCGACGACGACACACCTATGCTGCTCATCTTC | |
| | FILRQTEKHELLVL | GGCCATTTACCCCGGGTTCTGTTCAAGGCACCATACCA | |
| | VKKDQLNRHSYL | GCGGGACAACTTCATCCTGCGGCAGACCGAGAAGCAT | |
| | KDPDFLDAALDFN | GAGCTTCTGGTGCTGGTGAAGAAGGACCAGCTGAACC | |
| | YLDLSALLRNSFH | GGCACTCATATCTGAAGGACCCCGACTTCCTGGACGCC | |
| | RYAVDVLKSGRC | GCCCTGGACTTCAACTACCTGGATTTAAGCGCCCTGCT | |
| | QMLDRRTVEMAF | CCGTAACTCTTTCCACCGGTACGCCGTGGACGTGTTAA | |
| | AYALALFAAARQ | AGTCAGGCCGGTGCCAGATGTTGGACCGGCGGACCGT | |
| | EEAGAQVSVPRAL | GGAGATGGCCTTCGCTTACGCATTAGCCCTCTTCGCAG | |
| | DRQAALLQIQEFM | CCGCCCGGCAGGAGGAGGCCGGCGCGCAGGTGAGCGT | |
| | ITCLSQTPPRTTLL | GCCTAGAGCGTTGGATAGACAGGCGGCCTTGCTGCAG | |
| | LYPTAVDLAKRAL | ATCCAGGAGTTCATGATCACCTGCCTGAGCCAGACTCC | |
| | WTPNQITDITSLVR | TCCACGGACCACATTGCTGCTCTACCCCACCGCCGTTG | |
| | LVYILSKQNQQHL | ACCTGGCAAAGCGGGCGCTCTGGACTCCGAACCAGAT | |
| | IPQWALRQIADFA | CACCGACATCACCAGCCTGGTGCGGCTGGTGTACATCC | |
| | LKLHKTHLASFLS | TGAGCAAGCAGAATCAGCAGCACCTGATACCACAGTG | |
| | AFARQELYLMGSL | GGCACTACGCCAGATCGCCGACTTCGCCCTGAAGCTG | |
| | VHSMLVHTTERRE | CACAAGACCCACCTGGCCAGCTTCCTGTCTGCTTTCGC | |
| | IFIVETGLCSLAEL | AAGGCAGGAACTGTACCTGATGGGCTCTCTAGTGCAC | |
| | SHFTQLLAHPHHE | AGCATGCTCGTCCATACCACAGAGCGGCGGGAGATCT | |
| | YLSDLYTPCSSSG | TCATCGTGGAGACTGGCCTGTGCTCTCTTGCGGAACTG | |
| | RRDHSLERLTRLF | AGCCACTTCACCCAGCTCCTAGCCCACCCACACCACGA | |
| | PDATVPATVPAAL | GTACCTTTCTGACCTGTACACCCCGTGCTCATCAAGTG | |
| | SILSTMQPSTLETF | GACGGCGGGACCACTCGCTGGAAAGACTCACCCGGCT | |
| | PDLFCLPLGESFSA | GTTCCCCGACGCTACTGTGCCGGCAACTGTGCCTGCGG | |
| | LTVSEHVSYIVTN | CTCTCTCTATATTATCTACCATGCAGCCCAGCACACTC | |
| | QYLIKGISYPVSTT | GAAACCTTCCCTGATCTGTTCTGCCTGCCTCTAGGAGA | |
| | VVGQSLIITQTDSQ | GAGCTTCTCTGCCCTTACAGTGTCCGAGCACGTGAGCT | |
| | TKCELTRNMHTTH | ACATCGTGACAAACCAATACCTCATTAAGGGCATCAG | |
| | SITVALNISLENCA | CTACCCTGTTAGTACTACCGTCGTAGGCCAGAGCCTAA | |
| | FCQSALLEYDDTQ | TTATCACCCAGACCGACTCCCAGACAAAGTGCGAATT | |
| | GVINIMYMHDSDD | AACGCGCAACATGCACACAACCCACAGCATCACCGTG | |
| | VLFALDPYNEVVV | GCCCTGAACATTAGCCTCGAGAACTGCGCCTTCTGCCA | |
| | SSPRTHYLMLLKN | GAGTGCCTTGCTTGAGTATGATGATACCCAGGGCGTG | |
| | GTVLEVTDVVVD | ATCAACATCATGTACATGCACGACAGCGACGATGTGC | |
| | ATDSRLLMMSVY | TGTTCGCACTCGACCCCTACAACGAGGTGGTCGTAAGC | |
| | ALSAIIGIYLLYRM | AGTCCAAGGACCCATTATTTGATGCTGCTTAAGAACGG | |
| | LKTC (SEQ ID NO: | CACCGTGCTGGAGGTGACCGACGTGGTGGTAGACGCT | |
| | 59) | ACAGACTCCCGGCTGCTTATGATGAGCGTGTACGCGCT | |
| | | CAGTGCGATCATCGGCATCTACCTGCTTTATCGGATGC | |
| | | TAAAGACCTGC (SEQ ID NO: 110) | |
| SE_CMV_gH_ | MRPGLPSYLIILAV | ATGCGGCCCGGCTTGCCCAGCTACTTGATCATCTTGGC | C2/ |
| 039 | CLFSHLLSSRYGA | CGTGTGCTTGTTCAGCCACTTACTTAGCAGCCGGTACG | CAP1/ |
| | EAVSEPLDKAFHL | GCGCCGAGGCCGTGTCCGAGCCCCTCGACAAGGCCTT | T100 |
| | LLNTYGRPIRFLRE | CCACCTCCTCCTCAACACCTACGGCCGCCCCATCCGCT | |
| | NTTQCTYNSSLRN | TCCTCCGCGAGAACACCACCCAGTGCACCTACAACTCC | |
| | STVVRENAISFNFF | TCCCTCCGCAACTCCACCGTCGTGCGGGAGAATGCCAT | |
| | QSYNQYYVFHMP | CTCCTTCAACTTCTTCCAGTCCTACAACCAGTACTACG | |
| | RCLFAGPLAEQFL | TCTTCCACATGCCCCGCTGCCTCTTCGCCGGACCTCTC | |
| | NQVDLTETLERYQ | GCCGAGCAGTTCCTCAACCAGGTCGACCTCACCGAGA | |
| | QRLNTYALVSKDL | CGCTCGAGCGCTACCAGCAGAGGTTGAATACCTATGC | |
| | ASYRSFSQQLKAQ | CCTCGTCTCCAAGGACCTCGCCTCCTACCGCTCCTTCT | |
| | DSLGEQPTTVPPPI | CCCAGCAGCTCAAGGCCCAGGACTCCCTCGGCGAGCA | |
| | DLSIPHVWMPPQT | GCCCACCACCGTGCCTCCACCAATCGACCTCTCCATCC | |
| | TPHGWTESHTTSG | CGCACGTCTGGATGCCTCCCCAGACCACTCCGCACGGC | |
| | LHRPHFNQTCILFD | TGGACCGAGTCCCACACCACCTCCGGACTGCATCGCCC | |
| | GHDLLFSTVTPCL | TCACTTCAACCAGACCTGCATCCTCTTCGACGGCCACG | |
| | HQGFYLIDELRYV | ACCTCCTCTTCAGTACCGTGACGCCATGCCTGCACCAG | |
| | KITLTEDFFVVTVS | GGCTTCTACCTCATCGACGAGCTCCGCTACGTCAAGAT | |
| | IDDDTPMLLIFGHL | CACCCTTACCGAGGACTTCTTCGTCGTCACCGTCTCGA | |
| | PRVLFKAPYQRDN | TTGACGACGACACACCAATGCTCCTCATCTTCGGCCAC | |
| | FILRQTEKHELLVL | CTCCCGCGCGTGCTGTTCAAGGCGCCCTACCAGCGCGA | |
| | VKKDQLNRHSYL | CAACTTCATCCTGAGGCAGACCGAGAAGCACGAGCTG | |
| | KDPDFLDAALDFN | CTCGTCCTCGTCAAGAAGGACCAGCTCAACCGCCACTC | |
| | YLDLSALLRNSFH | CTACCTCAAGGACCCCGACTTCCTCGACGCCGCCCTCG | |
| | RYAVDVLKSGRC | ACTTCAACTACCTCGATCTGAGTGCTCTGCTGAGGAAT | |
| | QMLDRRTVEMAF | TCATTCCACCGCTACGCCGTCGACGTCCTCAAGTCCGG | |
| | AYALALFAAARQ | CCGCTGCCAGATGCTCGACCGCCGCACCGTCGAGATG | |
| | EEAGAQVSVPRAL | GCCTTCGCTTACGCGCTGGCACTCTTCGCTGCCGCCCG | |
| | DRQAALLQIQEFM | CCAGGAGGAGGCCGGCGCCCAGGTCAGTGTGCCAAGA | |
| | ITCLSQTPPRTTLL | GCACTGGATAGACAGGCCGCACTTTTGCAGATCCAGG | |
| | LYPTAVDLAKRAL | AGTTCATGATCACCTGCCTCTCCCAGACTCCGCCTCGC | |
| | WTPNQITDITSLVR | ACCACGTTGTTGTTGTACCCCACAGCAGTCGATCTGGC | |
| | LVYILSKQNQQHL | TAAGAGAGCCTTATGGACACCTAACCAGATCACCGAC | |
| | IPQWALRQIADFA | ATCACCTCCCTCGTTCGGCTGGTCTACATCCTCTCCAA | |
| | LKLHKTHLASFLS | GCAGAATCAGCAGCACCTGATCCCTCAGTGGGCACTC | |
| | AFARQELYLMGSL | CGTCAAATCGCCGACTTCGCCCTCAAGCTCCACAAGAC | |
| | VHSMLVHTTERRE | CCACCTGGCGTCTTTCCTCAGTGCATTCGCTAGGCAGG | |
| | IFIVETGLCSLAEL | AGCTGTACCTGATGGGCAGTCTCGTCCACTCCATGCTG | |
| | SHFTQLLAHPHHE | GTGCACACCACGGAGCGCCGCGAGATCTTCATCGTCG | |
| | YLSDLYTPCSSSG | AGACTGGCCTCTGTAGTTTAGCCGAGCTCAGTCACTTC | |
| | RRDHSLERLTRLF | ACCCAACTGCTCGCCCACCCTCACCACGAGTACCTTAG | |
| | PDATVPATVPAAL | TGACCTCTATACCCCTTGCTCTAGTTCCGGTCGCCGCG | |
| | SILSTMQPSTLETF | ACCACAGCCTGGAACGTCTGACCCGCCTCTTCCCCGAC | |
| | PDLFCLPLGESFSA | GCTACAGTACCAGCAACCGTGCCAGCGGCCCTGTCTAT | |
| | LTVSEHVSYIVTN | CCTGTCTACCATGCAGCCCTCCACCTTGGAGACGTTCC | |
| | QYLIKGISYPVSTT | CAGATCTGTTCTGCCTCCCTCTAGGCGAATCGTTCTCT | |
| | VVGQSLIITQTDSQ | GCACTCACGGTCAGCGAACACGTCTCCTACATCGTCAC | |
| | TKCELTRNMHTTH | AAACCAGTATCTGATAAAGGGCATCTCCTACCCAGTGT | |
| | SITVALNISLENCA | CGACCACTGTTGTCGGCCAGTCCCTCATCATCACACAG | |
| | FCQSALLEYDDTQ | ACTGATTCTCAGACTAAGTGCGAGTTGACACGGAACA | |
| | GVINIMYMHDSDD | TGCATACTACACATTCTATCACCGTTGCTTTAAACATA | |
| | VLFALDPYNEVVV | AGCCTGGAGAACTGCGCCTTCTGCCAGTCCGCTCTGCT | |
| | SSPRTHYLMLLKN | CGAGTACGACGATACGCAGGGCGTCATCAACATCATG | |
| | GTVLEVTDVVVD | TACATGCACGACTCCGATGACGTTCTGTTCGCACTGGA | |
| | ATDSRLLMMSVY | CCCCTACAACGAGGTCGTCGTCTCCTCTCCTAGGACTC | |
| | ALSAIIGIYLLYRM | ACTACTTAATGCTGTTGAAGAACGGCACCGTCCTCGAG | |
| | LKTC (SEQ ID NO: | GTCACCGACGTGGTCGTCGATGCTACAGACAGCCGAC | |
| | 59) | TGCTCATGATGTCCGTGTACGCTCTCTCCGCCATCATC | |
| | | GGCATCTACCTGCTCTACCGCATGCTCAAGACCTGC | |
| | | (SEQ ID NO: 111) | |
| SE_CMV_gH_ | MRPGLPSYLIILAV | ATGCGGCCCGGCCTACCCAGCTACCTTATCATCTTAGC | C2/ |
| 040 | CLFSHLLSSRYGA | CGTGTGCTTGTTCAGCCACTTGCTCAGCAGCCGGTACG | CAP1/ |
| | EAVSEPLDKAFHL | GCGCCGAGGCCGTGTCCGAGCCCCTCGACAAGGCCTT | T100 |
| | LLNTYGRPIRFLRE | CCACCTCCTCCTCAACACCTACGGCCGCCCCATCCGCT | |
| | NTTQCTYNSSLRN | TCCTCCGCGAGAACACCACCCAGTGCACCTACAACTCC | |
| | STVVRENAISFNFF | TCCCTCCGCAACTCCACCGTCGTCAGAGAGAATGCCAT | |
| | QSYNQYYVFHMP | CTCCTTCAACTTCTTCCAGTCCTACAACCAGTACTACG | |
| | RCLFAGPLAEQFL | TCTTCCACATGCCCCGCTGCCTCTTCGCCGGCCCACTC | |
| | NQVDLTETLERYQ | GCCGAGCAGTTCCTCAACCAGGTCGACCTCACCGAAA | |
| | QRLNTYALVSKDL | CTCTCGAGCGCTACCAGCAGCGGCTGAATACGTATGC | |
| | ASYRSFSQQLKAQ | CCTCGTCTCCAAGGACCTCGCCTCCTACCGCTCCTTCT | |
| | DSLGEQPTTVPPPI | CCCAGCAGCTCAAGGCCCAGGACTCCCTCGGCGAGCA | |
| | DLSIPHVWMPPQT | GCCCACCACCGTCCCTCCTCCAATCGACCTCTCCATCC | |
| | TPHGWTESHTTSG | CACACGTCTGGATGCCTCCCCAGACCACTCCTCACGGC | |
| | LHRPHFNQTCILFD | TGGACCGAGTCCCACACCACCTCCGGCCTCCACAGGC | |
| | GHDLLFSTVTPCL | CACACTTCAACCAGACCTGCATCCTCTTCGACGGCCAC | |
| | HQGFYLIDELRYV | GACCTCCTCTTCAGCACAGTGACGCCATGTCTGCATCA | |
| | KITLTEDFFVVTVS | GGGCTTCTACCTCATCGACGAGCTCCGCTACGTCAAGA | |
| | IDDDTPMLLIFGHL | TCACTCTGACGGAGGACTTCTTCGTCGTCACCGTCTCT | |
| | PRVLFKAPYQRDN | ATAGATGACGACACCCCAATGCTCCTCATCTTCGGCCA | |
| | FILRQTEKHELLVL | CTTGCCTCGCGTGCTGTTCAAGGCTCCCTACCAGCGCG | |
| | VKKDQLNRHSYL | ACAACTTCATCCTCCGCCAGACCGAGAAGCATGAATT | |
| | KDPDFLDAALDFN | ACTCGTCCTCGTCAAGAAGGACCAGCTCAACCGCCAC | |
| | YLDLSALLRNSFH | TCCTACCTCAAGGACCCCGACTTCCTCGACGCCGCCCT | |
| | RYAVDVLKSGRC | CGACTTCAACTACCTGGATCTTAGCGCCCTGCTGCGTA | |
| | QMLDRRTVEMAF | ACAGCTTCCACCGCTACGCCGTCGACGTCCTCAAGTCC | |
| | AYALALFAAARQ | GGCCGCTGCCAGATGCTCGACCGCCGCACCGTCGAGA | |
| | EEAGAQVSVPRAL | TGGCCTTCGCCTATGCTCTGGCCCTGTTCGCTGCCGCC | |
| | DRQAALLQIQEFM | CGCCAGGAGGAGGCCGGCGCCCAGGTTTCCGTTCCTC | |
| | ITCLSQTPPRTTLL | GGGCTTTAGACAGACAGGCTGCCCTGCTTCAGATCCA | |
| | LYPTAVDLAKRAL | GGAGTTCATGATCACCTGCCTCTCCCAGACGCCACCAC | |
| | WTPNQITDITSLVR | GCACCACACTGTTGCTGTACCCGACAGCAGTGGACCT | |
| | LVYILSKQNQQHL | GGCAAAGAGGGCACTCTGGACTCCAAACCAGATCACC | |
| | IPQWALRQIADFA | GACATCACCTCCCTCGTCCGCCTCGTCTACATCCTCTC | |
| | LKLHKTHLASFLS | CAAGCAGAACCAGCAACACCTGATACCTCAGTGGGCT | |
| | AFARQELYLMGSL | CTGCGGCAGATCGCCGACTTCGCCCTCAAGCTCCACAA | |
| | VHSMLVHTTERRE | GACCCATTTGGCCTCCTTCCTGAGTGCCTTCGCTAGAC | |
| | IFIVETGLCSLAEL | AGGAGCTGTACCTGATGGGCTCCCTGGTGCACTCCATG | |
| | SHFTQLLAHPHHE | CTGGTTCACACCACAGAGCGCCGCGAGATCTTCATCGT | |
| | YLSDLYTPCSSSG | CGAGACAGGCCTCTGTTCATTGGCAGAACTCTCGCATT | |
| | RRDHSLERLTRLF | TCACCCAGCTGCTGGCCCACCCGCACCACGAGTATCTG | |
| | PDATVPATVPAAL | AGTGACCTCTACACACCATGCAGTTCGTCAGGAAGGC | |
| | SILSTMQPSTLETF | GCGACCACAGCCTGGAGCGACTGACCCGCCTCTTCCCC | |
| | PDLFCLPLGESFSA | GACGCAACAGTCCCTGCTACTGTCCCTGCCGCGCTGAG | |
| | LTVSEHVSYIVTN | TATTTTATCAACGATGCAGCCCTCCACGCTCGAAACCT | |
| | QYLIKGISYPVSTT | TCCCTGACTTGTTCTGCCTCCCTCTTGGAGAGAGTTTC | |
| | VVGQSLIITQTDSQ | AGTGCCCTGACCGTGTCAGAGCACGTCTCCTACATCGT | |
| | TKCELTRNMHTTH | CACGAACCAGTATCTTATCAAGGGCATCTCCTACCCAG | |
| | SITVALNISLENCA | TATCGACTACAGTGGTTGGCCAGTCCCTCATCATCACC | |
| | FCQSALLEYDDTQ | CAGACTGACAGTCAAACTAAGTGCGAGCTTACTAGAA | |
| | GVINIMYMHDSDD | ACATGCACACAACCCACTCCATCACCGTTGCATTAAAC | |
| | VLFALDPYNEVVV | ATCAGCCTCGAGAACTGCGCCTTCTGCCAGAGCGCTCT | |
| | SSPRTHYLMLLKN | GCTGGAGTACGACGACACACAGGGCGTCATCAACATC | |
| | GTVLEVTDVVVD | ATGTACATGCACGACTCCGACGACGTCCTGTTCGCGCT | |
| | ATDSRLLMMSVY | TGATCCCTACAACGAGGTCGTCGTGTCCAGTCCACGAA | |
| | ALSAIIGIYLLYRM | CTCATTACCTCATGCTGCTTAAGAACGGCACCGTCCTC | |
| | LKTC (SEQ ID NO: | GAGGTCACCGACGTGGTCGTCGATGCTACCGACTCCC | |
| | 59) | GCCTCCTCATGATGTCCGTTTACGCACTCTCTGCGATC | |
| | | ATCGGCATCTACTTACTATATCGCATGCTCAAGACCTG | |
| | | C (SEQ ID NO: 112) | |
| SE_CMV_gL_ | MCRRPDCGFSFSP | ATGTGCAGAAGACCAGACTGCGGATTCAGCTTCTCCCC | C2/ |
| 041 | GPVILLWCCLLLPI | AGGCCCAGTGATCTTACTTTGGTGCTGCCTCCTCCTTC | CAP1/ |
| | VSSAAVSVAPTAA | CGATTGTGAGCAGTGCCGCCGTGAGCGTGGCTCCTACC | T100 |
| | EKVPAECPELTRR | GCGGCCGAGAAGGTGCCGGCCGAGTGCCCAGAGCTCA | |
| | CLLGEVFEGDKYE | CCCGAAGGTGCCTTCTGGGCGAGGTTTTCGAGGGAGA | |
| | SWLRPLVNVTGR | CAAGTACGAGTCTTGGCTCCGGCCTCTGGTCAACGTGA | |
| | DGPLSQLIRYRPVT | CGGGCAGAGACGGCCCTTTGAGCCAGCTCATCAGATA | |
| | PEAANSVLLDEAF | CAGACCAGTAACACCTGAGGCCGCCAATTCCGTCCTG | |
| | LDTLALLYNNPDQ | CTGGATGAGGCCTTCCTGGACACCCTCGCCCTCCTTTA | |
| | LRALLTLLSSDTAP | CAATAACCCAGACCAGCTGAGAGCCCTGTTGACCCTTC | |
| | RWMTVMRGYSEC | TCAGCAGCGACACCGCTCCACGGTGGATGACCGTCAT | |
| | GDGSPAVYTCVD | GAGAGGCTATAGCGAGTGCGGAGATGGCAGCCCTGCC | |
| | DLCRGYDLTRLSY | GTCTATACCTGCGTTGACGACCTGTGCAGGGGCTACGA | |
| | GRSIFTEHVLGFEL | TTTGACAAGGCTCAGCTACGGCAGATCTATATTCACAG | |
| | VPPSLFNVVVAIR | AGCATGTGCTGGGCTTCGAGCTGGTGCCGCCATCCCTG | |
| | NEATRTNRAVRLP | TTCAACGTGGTGGTCGCTATAAGGAACGAGGCCACCA | |
| | VSTAAAPEGITLFY | GAACAAATCGCGCCGTGAGACTGCCGGTGTCCACGGC | |
| | GLYNAVKEFCLRH | AGCCGCACCTGAGGGCATTACACTGTTCTATGGCCTCT | |
| | QLDPPLLRHLDKY | ACAACGCCGTGAAGGAGTTCTGTTTGCGGCACCAGCT | |
| | YAGLPPELKQTRV | GGACCCACCACTGCTCAGACACCTGGATAAGTACTAC | |
| | NLPAHSRYGPQAV | GCTGGCCTGCCTCCGGAGCTGAAGCAAACACGTGTGA | |
| | DAR (SEQ ID NO: | ATCTGCCAGCCCACTCTCGGTACGGACCGCAGGCCGT | |
| | 61) | GGACGCCCGG (SEQ ID NO: 113) | |
| SE_CMV_gL_ | MCRRPDCGFSFSP | ATGTGCAGGAGGCCAGACTGCGGCTTCTCATTCAGCCC | C2/ |
| 042 | GPVILLWCCLLLPI | AGGCCCAGTCATCCTCCTTTGGTGTTGCCTCCTTCTCCC | CAP1/ |
| | VSSAAVSVAPTAA | TATAGTTAGCAGTGCCGCCGTGAGCGTGGCCCCTACA | T100 |
| | EKVPAECPELTRR | GCCGCGGAGAAGGTGCCAGCGGAGTGCCCGGAGTTAA | |
| | CLLGEVFEGDKYE | CCAGACGTTGCCTCTTGGGCGAGGTGTTCGAGGGCGA | |
| | SWLRPLVNVTGR | TAAGTATGAGTCCTGGCTGCGGCCTCTGGTGAACGTGA | |
| | DGPLSQLIRYRPVT | CCGGCAGAGACGGACCTCTGTCCCAGCTGATCAGATA | |
| | PEAANSVLLDEAF | CAGACCAGTGACCCCTGAAGCCGCAAACAGCGTGCTG | |
| | LDTLALLYNNPDQ | CTGGACGAGGCCTTCCTGGACACCCTGGCCCTGTTATA | |
| | LRALLTLLSSDTAP | CAACAACCCTGACCAGCTTCGCGCGCTGCTTACACTGC | |
| | RWMTVMRGYSEC | TGAGCAGCGATACCGCCCCAAGATGGATGACTGTGAT | |
| | GDGSPAVYTCVD | GAGGGGATATAGCGAGTGTGGCGACGGCAGCCCTGCC | |
| | DLCRGYDLTRLSY | GTCTACACCTGTGTGGACGACCTCTGCAGAGGCTATGA | |
| | GRSIFTEHVLGFEL | CCTGACCAGACTGTCATACGGCCGAAGCATCTTCACCG | |
| | VPPSLFNVVVAIR | AGCACGTCTTAGGATTCGAGCTGGTGCCTCCAAGCCTC | |
| | NEATRTNRAVRLP | TTCAATGTGGTGGTGGCCATTCGGAACGAGGCTACCA | |
| | VSTAAAPEGITLFY | GAACCAACCGGGCGGTGCGTCTTCCAGTTTCTACAGCC | |
| | GLYNAVKEFCLRH | GCCGCCCCGGAAGGAATTACCCTGTTCTACGGCCTGTA | |
| | QLDPPLLRHLDKY | CAACGCTGTCAAGGAGTTCTGCCTGAGACACCAGCTG | |
| | YAGLPPELKQTRV | GATCCACCGCTGCTGCGCCACTTGGACAAGTACTATGC | |
| | NLPAHSRYGPQAV | GGGCCTCCCTCCTGAGCTCAAGCAGACGAGGGTGAAC | |
| | DAR (SEQ ID NO: | CTCCCTGCTCACTCACGTTATGGACCACAGGCCGTGGA | |
| | 61) | CGCTAGA (SEQ ID NO: 114) | |
| SE_CMV_gL_ | MCRRPDCGFSFSP | ATGTGCAGAAGGCCAGACTGCGGCTTCAGCTTCTCTCC | C2/ |
| 043 | GPVILLWCCLLLPI | AGGACCAGTGATCCTCCTCTGGTGCTGCCTTCTCCTCC | CAP1/ |
| | VSSAAVSVAPTAA | CTATTGTGTCCTCCGCCGCCGTGTCCGTGGCCCCAACC | T100 |
| | EKVPAECPELTRR | GCCGCCGAGAAGGTGCCAGCGGAGTGCCCAGAGCTCA | |
| | CLLGEVFEGDKYE | CCAGGCGCTGTCTGCTGGGCGAGGTGTTCGAGGGCGA | |
| | SWLRPLVNVTGR | TAAGTACGAGAGTTGGCTGAGGCCGTTGGTGAACGTG | |
| | DGPLSQLIRYRPVT | ACGGGCAGGGACGGCCCGCTAAGTCAGTTAATAAGGT | |
| | PEAANSVLLDEAF | ACCGGCCAGTGACCCCGGAGGCCGCCAACAGCGTGCT | |
| | LDTLALLYNNPDQ | GCTGGATGAGGCCTTCTTGGACACCCTGGCCCTGTTGT | |
| | LRALLTLLSSDTAP | ACAACAACCCAGACCAGCTGAGAGCCCTGCTGACTCT | |
| | RWMTVMRGYSEC | GTTGAGCAGCGACACCGCCCCAAGATGGATGACCGTG | |
| | GDGSPAVYTCVD | ATGAGAGGCTATAGCGAGTGCGGCGATGGCAGCCCTG | |
| | DLCRGYDLTRLSY | CCGTGTACACGTGCGTGGACGATTTGTGTAGAGGCTAC | |
| | GRSIFTEHVLGFEL | GACCTCACCAGACTGAGCTACGGCAGAAGCATCTTCA | |
| | VPPSLFNVVVAIR | CTGAGCATGTGCTGGGATTCGAGCTGGTGCCTCCTAGC | |
| | NEATRTNRAVRLP | CTGTTCAATGTTGTGGTGGCTATACGCAACGAGGCCAC | |
| | VSTAAAPEGITLFY | AAGAACAAACAGGGCCGTAAGACTCCCAGTGAGCACC | |
| | GLYNAVKEFCLRH | GCTGCAGCCCCTGAGGGAATCACGCTGTTCTACGGCCT | |
| | QLDPPLLRHLDKY | CTACAACGCTGTGAAGGAGTTCTGTCTGAGGCACCAA | |
| | YAGLPPELKQTRV | CTGGACCCACCTCTGCTTAGACACCTGGATAAGTACTA | |
| | NLPAHSRYGPQAV | CGCCGGCCTCCCACCTGAACTGAAGCAGACCAGGGTG | |
| | DAR (SEQ ID NO: | AATCTTCCTGCACACTCAAGGTATGGCCCACAGGCCGT | |
| | 61) | GGATGCCAGG (SEQ ID NO: 115) | |
| SE_CMV_gL_ | MCRRPDCGFSFSP | ATGTGCAGGCGGCCAGACTGCGGCTTCAGCTTCTCACC | C2/ |
| 044 | GPVILLWCCLLLPI | GGGCCCAGTCATCTTGTTGTGGTGCTGCCTCCTCCTCC | CAP1/ |
| | VSSAAVSVAPTAA | CTATCGTAAGCTCGGCAGCCGTCAGTGTGGCCCCAACC | T100 |
| | EKVPAECPELTRR | GCCGCCGAGAAGGTTCCAGCCGAGTGTCCAGAACTCA | |
| | CLLGEVFEGDKYE | CAAGGCGGTGCCTGCTGGGCGAGGTCTTCGAGGGCGA | |
| | SWLRPLVNVTGR | CAAGTATGAAAGCTGGCTCAGGCCACTTGTCAATGTG | |
| | DGPLSQLIRYRPVT | ACAGGCAGAGACGGCCCACTGAGCCAGCTTATCCGGT | |
| | PEAANSVLLDEAF | ATAGACCTGTCACTCCTGAGGCCGCTAACAGCGTGCTT | |
| | LDTLALLYNNPDQ | CTGGACGAGGCTTTCCTGGACACTCTGGCTCTGCTTTA | |
| | LRALLTLLSSDTAP | CAACAACCCAGACCAGCTGAGAGCCCTGCTGACCCTG | |
| | RWMTVMRGYSEC | CTGAGCAGCGATACAGCCCCAAGGTGGATGACAGTTA | |
| | GDGSPAVYTCVD | TGAGGGGATACAGCGAGTGTGGCGACGGAAGCCCAGC | |
| | DLCRGYDLTRLSY | CGTGTATACCTGCGTGGATGACCTGTGCAGAGGCTAC | |
| | GRSIFTEHVLGFEL | GACCTGACCCGCCTCTCCTACGGAAGATCCATCTTCAC | |
| | VPPSLFNVVVAIR | CGAGCACGTGCTAGGATTCGAGCTCGTCCCTCCTAGCC | |
| | NEATRTNRAVRLP | TGTTCAATGTGGTGGTGGCCATCAGAAACGAGGCCAC | |
| | VSTAAAPEGITLFY | TCGGACCAATAGAGCAGTGAGACTGCCAGTGAGCACC | |
| | GLYNAVKEFCLRH | GCGGCCGCACCAGAGGGTATCACACTGTTCTACGGCC | |
| | QLDPPLLRHLDKY | TGTACAACGCCGTGAAGGAGTTCTGTCTGCGTCACCAG | |
| | YAGLPPELKQTRV | CTGGACCCACCTCTGCTTAGACATCTGGATAAGTACTA | |
| | NLPAHSRYGPQAV | TGCCGGCCTGCCTCCTGAACTCAAGCAGACCCGTGTGA | |
| | DAR (SEQ ID NO: | ATCTGCCTGCCCACTCCAGATACGGCCCTCAGGCCGTG | |
| | 61) | GACGCAAGG (SEQ ID NO: 116) | |
| SE_CMV_gL_ | MCRRPDCGFSFSP | ATGTGTAGACGACCAGACTGCGGCTTCTCTTTCTCTCC | C2/ |
| 045 | GPVILLWCCLLLPI | AGGCCCGGTGATCTTACTCTGGTGTTGTTTGCTCCTTCC | CAP1/ |
| | VSSAAVSVAPTAA | TATCGTTAGTAGCGCCGCCGTGAGCGTGGCTCCGACA | T100 |
| | EKVPAECPELTRR | GCCGCCGAGAAGGTGCCAGCCGAGTGCCCAGAGCTCA | |
| | CLLGEVFEGDKYE | CCAGAAGATGTCTGCTGGGCGAGGTCTTCGAAGGCGA | |
| | SWLRPLVNVTGR | CAAGTACGAGTCTTGGCTGAGGCCTCTCGTGAATGTTA | |
| | DGPLSQLIRYRPVT | CCGGCAGGGACGGCCCACTGAGCCAGCTGATTAGGTA | |
| | PEAANSVLLDEAF | CCGGCCAGTGACCCCGGAAGCCGCCAACAGCGTGCTG | |
| | LDTLALLYNNPDQ | CTGGATGAAGCCTTCCTGGACACCCTGGCCCTGCTGTA | |
| | LRALLTLLSSDTAP | CAACAATCCTGACCAGCTCCGGGCCCTGCTGACCCTCC | |
| | RWMTVMRGYSEC | TCAGCAGCGACACTGCCCCTCGGTGGATGACAGTCAT | |
| | GDGSPAVYTCVD | GAGAGGCTACTCCGAATGTGGAGACGGCAGCCCTGCC | |
| | DLCRGYDLTRLSY | GTCTACACCTGTGTGGACGACCTCTGTAGGGGCTACGA | |
| | GRSIFTEHVLGFEL | CCTGACAAGACTGTCCTATGGCAGAAGCATTTTCACCG | |
| | VPPSLFNVVVAIR | AGCATGTGCTTGGCTTCGAGCTGGTGCCTCCATCCCTG | |
| | NEATRTNRAVRLP | TTCAACGTGGTTGTGGCCATTAGAAACGAGGCCACCA | |
| | VSTAAAPEGITLFY | GAACCAACAGGGCCGTGCGGCTGCCAGTGAGTACAGC | |
| | GLYNAVKEFCLRH | CGCTGCTCCAGAGGGCATTACCCTGTTCTACGGCCTTT | |
| | QLDPPLLRHLDKY | ACAATGCCGTGAAGGAGTTCTGTCTGCGCCATCAGCTG | |
| | YAGLPPELKQTRV | GACCCTCCTCTGCTGAGACACCTGGATAAGTATTACGC | |
| | NLPAHSRYGPQAV | GGGCCTGCCTCCAGAACTGAAGCAGACCCGCGTCAAC | |
| | DAR (SEQ ID NO: | CTGCCAGCTCATAGCCGTTACGGCCCGCAAGCAGTGG | |
| | 61) | ACGCCCGA (SEQ ID NO: 117) | |
| SE_CMV_gL_ | MCRRPDCGFSFSP | ATGTGCCGAAGACCGGACTGCGGCTTCAGCTTCAGCC | C2/ |
| 046 | GPVILLWCCLLLPI | CAGGCCCGGTTATCCTCCTCTGGTGCTGCCTCCTTCTCC | CAP1/ |
| | VSSAAVSVAPTAA | CAATCGTGAGCAGCGCCGCCGTGAGCGTGGCGCCTAC | T100 |
| | EKVPAECPELTRR | CGCCGCGGAGAAGGTCCCAGCCGAGTGCCCAGAATTG | |
| | CLLGEVFEGDKYE | ACGAGGAGATGCTTGCTGGGCGAAGTGTTCGAGGGCG | |
| | SWLRPLVNVTGR | ATAAGTATGAGAGCTGGCTGCGGCCTCTGGTCAACGT | |
| | DGPLSQLIRYRPVT | GACCGGCCGCGACGGCCCACTGTCCCAGCTGATCAGG | |
| | PEAANSVLLDEAF | TACAGACCAGTGACCCCAGAGGCTGCTAACAGCGTGC | |
| | LDTLALLYNNPDQ | TGCTGGATGAGGCTTTCCTCGACACGCTGGCTCTCCTG | |
| | LRALLTLLSSDTAP | TACAACAATCCGGATCAGCTCAGAGCCCTGCTCACACT | |
| | RWMTVMRGYSEC | GCTGTCCAGCGACACCGCTCCAAGGTGGATGACAGTG | |
| | GDGSPAVYTCVD | ATGCGGGGCTACTCAGAGTGCGGCGACGGTAGCCCTG | |
| | DLCRGYDLTRLSY | CGGTGTATACATGTGTGGACGACCTGTGCAGAGGCTA | |
| | GRSIFTEHVLGFEL | CGACTTAACCAGGCTGTCCTACGGTAGATCCATCTTCA | |
| | VPPSLFNVVVAIR | CTGAGCACGTGCTGGGCTTCGAGCTGGTGCCACCTAGC | |
| | NEATRTNRAVRLP | CTGTTCAATGTCGTGGTAGCCATCCGGAACGAGGCTAC | |
| | VSTAAAPEGITLFY | CAGAACAAATCGGGCCGTGAGGCTCCCAGTGAGCACC | |
| | GLYNAVKEFCLRH | GCCGCCGCTCCTGAGGGCATCACTCTGTTCTACGGACT | |
| | QLDPPLLRHLDKY | TTACAACGCCGTCAAGGAGTTCTGCCTGCGGCACCAG | |
| | YAGLPPELKQTRV | CTCGATCCACCTCTGCTGAGACACCTGGACAAGTACTA | |
| | NLPAHSRYGPQAV | CGCCGGCCTTCCGCCTGAGCTGAAGCAGACCAGAGTC | |
| | DAR (SEQ ID NO: | AACCTGCCTGCCCATAGCAGATACGGCCCACAGGCTG | |
| | 61) | TGGATGCCAGA (SEQ ID NO: 118) | |
| SE_CMV_gL_ | MCRRPDCGFSFSP | ATGTGCCGGCGGCCCGACTGCGGCTTCAGCTTCAGCCC | C2/ |
| 047 | GPVILLWCCLLLPI | CGGCCCCGTGATCCTATTGTGGTGCTGCCTACTTTTGC | CAP1/ |
| | VSSAAVSVAPTAA | CCATCGTGAGCAGCGCCGCCGTGAGCGTGGCTCCTAC | T100 |
| | EKVPAECPELTRR | CGCCGCCGAGAAGGTGCCCGCCGAGTGCCCCGAGCTA | |
| | CLLGEVFEGDKYE | ACCCGGCGGTGCCTTCTTGGCGAGGTGTTCGAGGGCG | |
| | SWLRPLVNVTGR | ACAAGTACGAGAGCTGGCTGCGGCCCCTGGTGAACGT | |
| | DGPLSQLIRYRPVT | GACCGGCCGGGACGGCCCTCTGAGCCAGCTGATCCGG | |
| | PEAANSVLLDEAF | TACCGGCCCGTGACACCAGAGGCCGCCAACAGCGTGC | |
| | LDTLALLYNNPDQ | TGCTGGACGAGGCCTTCCTGGACACCCTGGCCCTGCTG | |
| | LRALLTLLSSDTAP | TACAACAACCCCGACCAGCTGAGAGCTCTGCTGACGC | |
| | RWMTVMRGYSEC | TGCTGTCAAGCGACACCGCGCCACGGTGGATGACCGT | |
| | GDGSPAVYTCVD | GATGCGGGGCTACAGCGAGTGCGGCGACGGCAGCCCC | |
| | DLCRGYDLTRLSY | GCCGTGTACACCTGCGTGGACGACCTGTGCAGGGGCT | |
| | GRSIFTEHVLGFEL | ATGACCTGACCCGTCTGAGCTACGGCCGGAGCATCTTC | |
| | VPPSLFNVVVAIR | ACCGAGCACGTGCTGGGCTTCGAGCTGGTGCCTCCCA | |
| | NEATRTNRAVRLP | GCCTGTTCAACGTGGTGGTGGCCATCCGGAACGAGGC | |
| | VSTAAAPEGITLFY | CACCCGGACCAACCGGGCCGTGCGGCTGCCCGTGAGC | |
| | GLYNAVKEFCLRH | ACCGCAGCAGCCCCAGAAGGCATCACCCTGTTCTACG | |
| | QLDPPLLRHLDKY | GCCTGTACAATGCCGTGAAGGAGTTCTGCCTGCGGCA | |
| | YAGLPPELKQTRV | CCAGCTGGACCCACCTCTTCTCAGGCACCTGGATAAGT | |
| | NLPAHSRYGPQAV | ACTACGCCGGCCTGCCTCCTGAACTGAAGCAGACCCG | |
| | DAR (SEQ ID NO: | GGTGAACCTGCCCGCCCACAGCCGGTATGGCCCACAG | |
| | 61) | GCCGTGGACGCCCGG (SEQ ID NO: 119) | |
| SE_CMV_gL_ | MCRRPDCGFSFSP | ATGTGCCGGCGGCCCGACTGCGGCTTCAGCTTCAGCCC | C2/ |
| 048 | GPVILLWCCLLLPI | CGGCCCCGTGATCCTCCTCTGGTGCTGCCTCTTGTTGC | CAP1/ |
| | VSSAAVSVAPTAA | CCATCGTGAGCAGCGCCGCCGTGAGCGTGGCTCCTAC | T100 |
| | EKVPAECPELTRR | CGCCGCCGAGAAGGTGCCCGCCGAGTGCCCCGAGCTC | |
| | CLLGEVFEGDKYE | ACCCGGCGGTGCCTTCTGGGCGAGGTGTTCGAGGGCG | |
| | SWLRPLVNVTGR | ACAAGTACGAGAGCTGGCTGCGGCCCCTGGTGAACGT | |
| | DGPLSQLIRYRPVT | GACCGGCCGGGACGGCCCTCTGAGCCAGCTGATCCGG | |
| | PEAANSVLLDEAF | TACCGGCCCGTGACTCCAGAGGCCGCCAACAGCGTGC | |
| | LDTLALLYNNPDQ | TGCTGGACGAGGCCTTCCTGGACACCCTGGCCCTGCTG | |
| | LRALLTLLSSDTAP | TACAACAACCCCGACCAGCTGAGGGCCCTTCTGACCCT | |
| | RWMTVMRGYSEC | GCTCAGCAGCGACACCGCCCCACGGTGGATGACCGTG | |
| | GDGSPAVYTCVD | ATGCGGGGCTACAGCGAGTGCGGCGACGGCAGCCCCG | |
| | DLCRGYDLTRLSY | CCGTGTACACCTGCGTGGACGACCTGTGCAGGGGCTA | |
| | GRSIFTEHVLGFEL | CGACCTGACCAGGCTGAGCTACGGCCGGAGCATCTTC | |
| | VPPSLFNVVVAIR | ACCGAGCACGTGCTGGGCTTCGAGCTGGTGCCGCCCA | |
| | NEATRTNRAVRLP | GCCTGTTCAACGTGGTGGTGGCCATCCGGAACGAGGC | |
| | VSTAAAPEGITLFY | CACCCGGACCAACCGGGCCGTGCGGCTGCCCGTGTCG | |
| | GLYNAVKEFCLRH | ACAGCCGCCGCCCCTGAGGGCATCACCCTGTTCTACGG | |
| | QLDPPLLRHLDKY | CCTATATAACGCCGTGAAGGAGTTCTGCCTGCGGCACC | |
| | YAGLPPELKQTRV | AGCTGGACCCGCCCCTGCTTCGCCACCTGGACAAGTAT | |
| | NLPAHSRYGPQAV | TACGCCGGCCTGCCTCCGGAGCTGAAGCAGACCCGGG | |
| | DAR (SEQ ID NO: | TGAACCTGCCCGCCCACAGCCGGTACGGCCCTCAGGC | |
| | 61) | CGTGGACGCCCGG (SEQ ID NO: 120) | |
| SE_CMV_gL_ | MCRRPDCGFSFSP | ATGTGCCGGCGGCCCGACTGCGGCTTCAGCTTCAGCCC | C2/ |
| 049 | GPVILLWCCLLLPI | CGGCCCCGTGATCCTCCTTTGGTGCTGCTTGCTCTTGCC | CAP1/ |
| | VSSAAVSVAPTAA | CATCGTGAGCAGCGCCGCCGTCTCCGTCGCTCCTACCG | T100 |
| | EKVPAECPELTRR | CCGCCGAGAAGGTCCCCGCCGAGTGCCCCGAGCTCAC | |
| | CLLGEVFEGDKYE | CCGCCGCTGCCTCCTCGGCGAGGTCTTCGAGGGCGAC | |
| | SWLRPLVNVTGR | AAGTACGAGTCCTGGCTCAGACCTCTCGTCAACGTCAC | |
| | DGPLSQLIRYRPVT | CGGCCGCGACGGCCCACTCTCCCAGCTCATCCGCTACC | |
| | PEAANSVLLDEAF | GCCCCGTCACACCGGAGGCCGCCAACTCCGTCCTCCTC | |
| | LDTLALLYNNPDQ | GACGAGGCCTTCCTCGACACCCTCGCCCTCCTCTACAA | |
| | LRALLTLLSSDTAP | CAACCCCGACCAGCTCCGAGCCCTGCTGACCCTGCTGT | |
| | RWMTVMRGYSEC | CCTCCGACACCGCTCCTCGCTGGATGACCGTCATGCGC | |
| | GDGSPAVYTCVD | GGCTACTCCGAGTGCGGCGACGGCTCCCCAGCTGTGT | |
| | DLCRGYDLTRLSY | ACACCTGCGTCGACGACCTCTGCAGAGGCTACGACCT | |
| | GRSIFTEHVLGFEL | GACGCGCCTCTCCTACGGCCGCTCCATCTTCACCGAGC | |
| | VPPSLFNVVVAIR | ACGTCCTCGGCTTCGAGCTCGTGCCTCCCTCCCTCTTC | |
| | NEATRTNRAVRLP | AACGTCGTCGTCGCCATCCGCAACGAGGCCACCCGCA | |
| | VSTAAAPEGITLFY | CCAACCGCGCCGTCCGCCTCCCCGTCAGCACAGCCGCT | |
| | GLYNAVKEFCLRH | GCACCAGAGGGCATCACCCTCTTCTACGGACTGTACA | |
| | QLDPPLLRHLDKY | ACGCCGTCAAGGAGTTCTGCCTCCGCCACCAGCTCGAC | |
| | YAGLPPELKQTRV | CCACCTCTGCTGAGGCATCTGGACAAGTATTACGCCGG | |
| | NLPAHSRYGPQAV | CCTCCCACCAGAGCTGAAGCAGACCCGCGTCAACCTC | |
| | DAR (SEQ ID NO: | CCCGCCCACTCCCGCTACGGACCACAGGCCGTCGACG | |
| | 61) | CCCGC (SEQ ID NO: 121) | |
| SE_CMV_gL_ | MCRRPDCGFSFSP | ATGTGCCGGCGGCCCGACTGCGGCTTCAGCTTCAGCCC | C2/ |
| 050 | GPVILLWCCLLLPI | CGGCCCCGTGATCCTCCTCTGGTGCTGCCTTTTGCTCCC | CAP1/ |
| | VSSAAVSVAPTAA | CATCGTGAGCAGCGCCGCCGTCTCCGTCGCTCCTACCG | T100 |
| | EKVPAECPELTRR | CCGCCGAGAAGGTCCCCGCCGAGTGCCCCGAGCTCAC | |
| | CLLGEVFEGDKYE | CCGCCGCTGCCTCCTCGGCGAGGTCTTCGAGGGCGAC | |
| | SWLRPLVNVTGR | AAGTACGAGTCCTGGCTCAGACCTCTCGTCAACGTCAC | |
| | DGPLSQLIRYRPVT | CGGCCGCGACGGCCCACTCTCCCAGCTCATCCGCTACC | |
| | PEAANSVLLDEAF | GCCCCGTCACCCCAAGAGGCCGCCAACTCCGTCCTCCTC | |
| | LDTLALLYNNPDQ | GACGAGGCCTTCCTCGACACCCTCGCCCTCCTCTACAA | |
| | LRALLTLLSSDTAP | CAACCCCGACCAGCTCAGGGCCCTTCTAACCCTGCTGT | |
| | RWMTVMRGYSEC | CCTCCGACACCGCCCCTCGCTGGATGACCGTCATGCGC | |
| | GDGSPAVYTCVD | GGCTACTCCGAGTGCGGCGACGGCTCCCCGGCCGTGT | |
| | DLCRGYDLTRLSY | ACACCTGCGTCGACGACCTCTGCAGAGGATACGACCT | |
| | GRSIFTEHVLGFEL | CACCCGGCTCTCCTACGGCCGCTCCATCTTCACCGAGC | |
| | VPPSLFNVVVAIR | ACGTCCTCGGCTTCGAGCTCGTCCCACCCTCCCTCTTC | |
| | NEATRTNRAVRLP | AACGTCGTCGTCGCCATCCGCAACGAGGCCACCCGCA | |
| | VSTAAAPEGITLFY | CCAACCGCGCCGTCCGCCTCCCCGTCAGCACAGCCGC | |
| | GLYNAVKEFCLRH | AGCCCCAGAGGGCATCACCCTCTTCTACGGCCTGTATA | |
| | QLDPPLLRHLDKY | ACGCCGTCAAGGAGTTCTGCCTCCGCCACCAGCTCGAC | |
| | YAGLPPELKQTRV | CCGCCTCTGCTGAGGCACCTGGACAAGTATTACGCCG | |
| | NLPAHSRYGPQAV | GCCTCCCTCCTGAGCTGAAGCAGACCCGCGTCAACCTC | |
| | DAR (SEQ ID NO: | CCCGCCCACTCCCGCTATGGACCACAGGCCGTCGACG | |
| | 61) | CCCGC (SEQ ID NO: 122) | |
| SE_CMV_ Tr | MESRIWCLVVCV | ATGGAGAGCAGAATTTGGTGCTTGGTGGTGTGTGTTAA | C2/ |
| gB6XHis_051 | NLCIVCLGAAVSS | CCTCTGCATTGTTTGCCTCGGCGCAGCAGTGTCTAGCA | CAP1/ |
| (truncated gB | SSTRGTSATHSHH | GCTCTACTCGGGGAACATCCGCAACCCACAGCCACCA | T100 |
| with his6, all | SSHTTSAAHSRSG | TAGCTCACACACCACCAGCGCCGCCCACTCCAGATCC | |
| aa sequence | SVSQRVTSSQTVS | GGTAGCGTGAGCCAGCGGGTGACTAGCAGCCAGACTG | |
| the same) | HGVNETIYNTTLK | TGTCCCATGGCGTCAACGAGACAATCTACAACACCAC | |
| | YGDVVGVNTTKY | CCTGAAGTACGGCGACGTCGTGGGCGTGAACACAACG | |
| | PYRVCSMAQGTD | AAGTACCCTTACAGGGTGTGTAGCATGGCTCAGGGCA | |
| | LIRFERNIVCTSMK | CCGACTTGATCCGGTTCGAGAGAAATATTGTATGCACC | |
| | PINEDLDEGIMVV | AGCATGAAGCCAATTAACGAGGATCTGGACGAAGGCA | |
| | YKRNIVAHTFKVR | TCATGGTAGTGTATAAGAGAAACATAGTTGCACACAC | |
| | VYQKVLTFRRSYA | TTTCAAGGTGAGGGTCTACCAGAAGGTGCTGACCTTCC | |
| | YIHTTYLLGSNTE | GCCGAAGCTATGCTTACATCCATACTACCTACCTGCTC | |
| | YVAPPMWEIHHIN | GGTTCTAACACCGAGTATGTGGCACCTCCAATGTGGG | |
| | SHSQCYSSYSRVIA | AGATCCACCACATCAATTCTCATAGCCAGTGTTACAGC | |
| | GTVFVAYHRDSYE | TCTTACAGCCGGGTGATAGCCGGCACCGTCTTCGTGGC | |
| | NKTMQLMPDDYS | CTACCATAGAGATTCATACGAGAACAAGACCATGCAG | |
| | NTHSTRYVTVKD | CTGATGCCAGACGACTACAGCAACACCCATTCCACCA | |
| | QWHSRGSTWLYR | GGTATGTGACAGTCAAGGACCAATGGCACTCACGCGG | |
| | ETCNLNCMVTITT | CTCCACCTGGCTGTACAGGGAGACTTGCAACCTCAATT | |
| | ARSKYPYHFFATS | GCATGGTGACCATCACCACCGCCCGCAGCAAGTACCC | |
| | TGDVVDISPEYNG | GTACCACTTCTTCGCCACCAGCACCGGAGATGTCGTGG | |
| | TNRNASYFGENAD | ACATCAGCCCTTTCTATAACGGCACTAACAGAAACGC | |
| | KFFIFPNYTIVSDF | CAGTTACTTCGGAGAGAATGCCGACAAGTTCTTCATCT | |
| | GRPNSALETHRLV | TCCCGAACTACACTATTGTGAGCGATTTCGGTCGCCCT | |
| | AFLERADSVISWDI | AACTCCGCCCTGGAGACACACCGCCTGGTTGCCTTCCT | |
| | QDEKNVTCQLTF | GGAGAGAGCCGATTCCGTGATCAGCTGGGACATCCAG | |
| | WEASERTIRSEAE | GACGAGAAGAATGTGACCTGTCAGCTCACTTTCTGGG | |
| | DSYHFSSAKMTAT | AGGCCTCCGAGAGGACTATCCGGAGCGAGGCCGAGGA | |
| | FLSKKQEVNMSDS | CTCATACCATTTCAGCAGCGCCAAGATGACCGCCACCT | |
| | ALDCVRDEAINKL | TCCTGTCAAAGAAGCAGGAGGTGAACATGTCAGATAG | |
| | QQIFNTSYNQTYE | CGCTCTGGACTGTGTGCGCGACGAGGCTATTAACAAG | |
| | KYGNVSVFETTGG | CTGCAGCAGATCTTCAATACCTCCTACAATCAGACCTA | |
| | LVVFWQGIKQKSL | CGAGAAGTATGGTAACGTGTCAGTATTCGAGACAACT | |
| | VELERLANRSSLN | GGCGGCCTCGTGGTGTTCTGGCAGGGAATCAAGCAGA | |
| | LTHNRTKRSTDGN | AGTCCCTGGTGGAGCTTGAAAGACTCGCCAACCGGAG | |
| | NATHISNMESVH | CAGCCTGAACCTGACCCACAACAGGACAAAGAGATCT | |
| | NLVYAQLQFTYDT | ACAGATGGTAACAACGCCACCCATCTGAGCAACATGG | |
| | LRGYINRALAQIA | AGTCCGTGCACAACCTGGTGTACGCCCAGCTCCAGTTC | |
| | EAWCVDQRRTLE | ACATACGACACCCTGAGAGGCTACATTAATAGAGCCC | |
| | VFKELSKINPSAIL | TCGCCCAAATCGCAGAGGCCTGGTGCGTGGACCAGAG | |
| | SAIYNKPIAARFM | GCGAACCCTGGAGGTGTTCAAGGAATTGAGCAAGATC | |
| | GDVLGLASCVTIN | AATCCAAGCGCCATCTTGAGCGCAATCTATAACAAGC | |
| | QTSVKVLRDMNV | CGATTGCGGCCAGATTCATGGGCGACGTGTTGGGCCT | |
| | KESPGRCYSRPVVI | GGCCTCCTGCGTCACTATCAACCAGACCTCTGTCAAGG | |
| | FNFANSSYVQYGQ | TGCTCAGAGATATGAACGTTAAGGAGTCCCCAGGCAG | |
| | LGEDNEILLGNHR | ATGCTATAGCAGACCTGTCGTGATTTTCAATTTCGCCA | |
| | TEECQLPSLKIFIA | ACTCAAGCTACGTGCAGTACGGCCAGCTCGGCGAAGA | |
| | GNSAYEYVDYLF | TAACGAGATCCTGCTGGGCAACCACAGAACCGAAGAG | |
| | KRMIDLSSISTVDS | TGCCAGCTGCCTTCCCTGAAGATTTTCATCGCTGGCAA | |
| | MIALDIDPLENTDF | CTCCGCTTACGAGTACGTGGATTACCTGTTCAAGAGAA | |
| | RVLELYSQKELRS | TGATCGACCTCAGCAGCATCAGCACCGTGGACAGCAT | |
| | SNVFDLEEIMREF | GATCGCCTTAGACATTGACCCTCTGGAGAACACAGATT | |
| | NSYKQHHHHHH | TCAGGGTGCTGGAACTATACTCTCAGAAGGAGCTCCG | |
| | (SEQ ID NO: 83) | GTCTAGCAACGTGTTCGATCTGGAGGAGATCATGCGG | |
| | | GAGTTCAATTCCTACAAGCAGCACCACCACCATCATCA | |
| | | C (SEQ ID NO: 123) | |
| SE_CMV_ | MESRIWCLVVCV | ATGGAGTCAAGAATCTGGTGTCTTGTGGTGTGCGTGAA | C2/ |
| TrgB6XHis_052 | NLCIVCLGAAVSS | CTTGTGTATCGTGTGTTTGGGCGCAGCCGTGTCCTCAA | CAP1/ |
| | SSTRGTSATHSHH | GCAGCACTAGAGGCACTAGCGCCACCCACTCCCATCA | T100 |
| | SSHTTSAAHSRSG | TAGCTCACACACCACAAGCGCCGCCCACTCTAGGTCA | |
| | SVSQRVTSSQTVS | GGCAGCGTGTCTCAGCGCGTGACCTCTAGCCAGACTGT | |
| | HGVNETIYNTTLK | GAGTCACGGAGTCAATGAAACCATCTACAACACAACA | |
| | YGDVVGVNTTKY | CTGAAGTACGGAGACGTCGTGGGCGTGAATACAACCA | |
| | PYRVCSMAQGTD | AGTACCCATACAGGGTGTGCAGCATGGCTCAGGGCAC | |
| | LIRFERNIVCTSMK | TGACCTCATCAGATTCGAGCGGAACATTGTGTGCACCT | |
| | PINEDLDEGIMVV | CAATGAAGCCTATCAACGAGGATTTGGATGAAGGCAT | |
| | YKRNIVAHTFKVR | CATGGTCGTGTACAAGAGAAACATCGTCGCTCATACCT | |
| | VYQKVLTFRRSYA | TCAAGGTGAGAGTGTATCAGAAGGTGCTGACCTTCAG | |
| | YIHTTYLLGSNTE | ACGTAGCTACGCTTACATTCACACCACCTACCTGCTGG | |
| | YVAPPMWEIHHIN | GCAGCAACACCGAGTATGTGGCTCCTCCTATGTGGGA | |
| | SHSQCYSSYSRVIA | GATACATCACATCAACAGCCATTCTCAGTGCTATAGTT | |
| | GTVFVAYHRDSYE | CTTATAGCAGGGTCATCGCCGGCACCGTCTTCGTGGCC | |
| | NKTMQLMPDDYS | TACCATAGAGATAGCTACGAGAACAAGACCATGCAGT | |
| | NTHSTRYVTVKD | TGATGCCGGACGATTACAGCAATACCCATAGCACTAG | |
| | QWHSRGSTWLYR | GTACGTGACTGTGAAGGACCAGTGGCACTCCCGGGGT | |
| | ETCNLNCMVTITT | AGCACCTGGCTTTACAGGGAAACCTGCAATCTGAACT | |
| | ARSKYPYHFFATS | GCATGGTGACTATTACCACCGCCAGGAGCAAGTATCC | |
| | TGDVVDISPFYNG | TTACCACTTCTTCGCTACATCTACTGGAGACGTGGTCG | |
| | TNRNASYFGENAD | ATATCTCTCCTTTCTACAATGGCACAAACAGAAATGCT | |
| | KFFIFPNYTIVSDF | TCATATTTCGGCGAGAATGCCGACAAGTTCTTCATCTT | |
| | GRPNSALETHRLV | CCCAAACTACACCATTGTGTCCGACTTCGGAAGACCTA | |
| | AFLERADSVISWDI | ATTCCGCCCTGGAAACCCATAGACTGGTCGCATTCCTG | |
| | QDEKNVTCQLTF | GAAAGGGCCGACTCCGTCATTTCATGGGACATCCAGG | |
| | WEASERTIRSEAE | ATGAGAAGAACGTCACCTGTCAGCTCACATTCTGGGA | |
| | DSYHFSSAKMTAT | AGCGAGCGAAAGAACAATTCGCAGCGAAGCCGAGGA | |
| | FLSKKQEVNMSDS | CAGCTATCATTTCAGCAGTGCTAAGATGACCGCCACTT | |
| | ALDCVRDEAINKL | TCCTGTCTAAGAAGCAGGAGGTGAACATGTCCGACAG | |
| | QQIFNTSYNQTYE | CGCCTTGGATTGCGTGAGAGACGAAGCTATTAACAAG | |
| | KYGNVSVFETTGG | CTGCAGCAGATCTTCAACACCTCCTACAACCAGACTTA | |
| | LVVFWQGIKQKSL | CGAGAAGTATGGCAATGTGAGTGTGTTCGAGACAACC | |
| | VELERLANRSSLN | GGCGGCCTGGTAGTATTCTGGCAGGGCATCAAGCAGA | |
| | LTHNRTKRSTDGN | AGTCACTGGTGGAGCTTGAGAGGCTGGCCAATAGATC | |
| | NATHLSNMESVH | CAGCCTGAACCTGACCCACAACCGGACAAAGAGATCT | |
| | NLVYAQLQFTYDT | ACCGACGGAAACAACGCCACTCATCTTTCCAATATGG | |
| | LRGYINRALAQIA | AGAGCGTGCACAACCTGGTGTACGCGCAGCTCCAGTT | |
| | EAWCVDQRRTLE | CACCTACGACACACTGAGGGGCTACATAAACAGGGCC | |
| | VFKELSKINPSAIL | CTGGCACAGATCGCCGAAGCCTGGTGCGTGGACCAGA | |
| | SAIYNKPIAARFM | GAAGGACCCTGGAGGTTTTCAAGGAGCTGAGCAAGAT | |
| | GDVLGLASCVTIN | TAATCCGTCCGCTATCCTGAGCGCAATATACAATAAGC | |
| | QTSVKVLRDMNV | CAATCGCCGCCAGATTCATGGGCGACGTGTTGGGACT | |
| | KESPGRCYSRPVVI | GGCCAGTTGCGTCACAATAAACCAGACCTCTGTAAAG | |
| | FNFANSSYVQYGQ | GTCCTGAGGGACATGAATGTCAAGGAGAGCCCGGGCA | |
| | LGEDNEILLGNHR | GGTGCTACAGCCGTCCTGTGGTGATTTTCAACTTCGCT | |
| | TEECQLPSLKIFIA | AATTCATCTTACGTCCAGTACGGCCAGCTGGGCGAAG | |
| | GNSAYEYVDYLF | ACAATGAGATCTTACTGGGCAACCATAGGACTGAGGA | |
| | KRMIDLSSISTVDS | GTGCCAGCTGCCGAGTCTGAAGATTTTCATAGCCGGCA | |
| | MIALDIDPLENTDF | ATAGCGCATATGAATATGTAGACTACCTGTTCAAGAG | |
| | RVLELYSQKELRS | GATGATTGACCTCTCTAGCATCTCGACCGTGGACAGCA | |
| | SNVFDLEEIMREF | TGATCGCCCTCGACATCGACCCTCTGGAGAACACAGA | |
| | NSYKQHHHHHH | CTTCCGGGTCCTCGAACTGTACAGCCAGAAGGAGCTT | |
| | (SEQ ID NO: 83) | AGGAGCTCCAACGTGTTCGATCTTGAGGAGATCATGA | |
| | | GGGAGTTCAATAGCTATAAGCAACATCACCACCATCA | |
| | | TCAC (SEQ ID NO: 124) | |
| SE_CMV_ | MESRIWCLVVCV | ATGGAGAGCAGAATTTGGTGTCTCGTGGTGTGTGTTAA | C2/ |
| TrgB6XHis_053 | NLCIVCLGAAVSS | TCTCTGCATCGTGTGCCTTGGCGCCGCCGTGTCCAGCA | CAP1/ |
| | SSTRGTSATHSHH | GCTCCACCAGGGGCACCAGCGCAACACACAGCCACCA | T100 |
| | SSHTTSAAHSRSG | CTCTAGCCACACAACCAGCGCCGCCCACTCTCGTTCAG | |
| | SVSQRVTSSQTVS | GCTCCGTTTCACAGAGGGTGACCTCCTCTCAAACCGTG | |
| | HGVNETIYNTTLK | TCTCATGGAGTGAATGAAACCATTTATAACACAACACT | |
| | YGDVVGVNTTKY | GAAGTATGGCGACGTGGTGGGCGTCAACACCACCAAG | |
| | PYRVCSMAQGTD | TATCCTTACCGGGTTTGTTCAATGGCCCAGGGCACCGA | |
| | LIRFERNIVCTSMK | TCTCATCAGATTCGAGCGAAATATCGTGTGTACATCCA | |
| | PINEDLDEGIMVV | TGAAGCCTATCAACGAAGACCTGGACGAGGGAATTAT | |
| | YKRNIVAHTFKVR | GGTCGTGTACAAGAGAAATATTGTGGCCCACACTTTCA | |
| | VYQKVLTFRRSYA | AGGTGAGAGTGTACCAGAAGGTGTTGACATTCAGGCG | |
| | YIHTTYLLGSNTE | GTCCTACGCCTACATCCACACCACTTATCTGTTGGGAT | |
| | YVAPPMWEIHHIN | CCAACACAGAGTACGTCGCACCGCCTATGTGGGAAAT | |
| | SHSQCYSSYSRVIA | ACATCACATCAATTCCCATTCTCAGTGCTATTCTAGCT | |
| | GTVFVAYHRDSYE | ACTCCAGAGTGATCGCCGGCACCGTGTTCGTGGCCTAC | |
| | NKTMQLMPDDYS | CACCGCGATAGCTACGAGAATAAGACCATGCAGCTGA | |
| | NTHSTRYVTVKD | TGCCTGACGATTACAGCAACACTCATTCCACACGCTAC | |
| | QWHSRGSTWLYR | GTGACCGTGAAGGATCAGTGGCACAGCCGCGGCAGCA | |
| | ETCNLNCMVTITT | CCTGGCTGTACCGGGAAACCTGCAACCTGAACTGCAT | |
| | ARSKYPYHFFATS | GGTGACAATAACAACCGCACGTAGCAAGTACCCATAC | |
| | TGDVVDISPFYNG | CACTTCTTCGCCACCTCCACCGGTGACGTGGTCGACAT | |
| | TNRNASYFGENAD | CAGCCCTTTCTACAATGGCACCAACAGAAATGCCTCCT | |
| | KFFIFPNYTIVSDF | ACTTCGGCGAGAACGCCGACAAGTTCTTCATCTTCCCT | |
| | GRPNSALETHRLV | AATTATACAATTGTGAGCGACTTCGGCAGGCCTAACA | |
| | AFLERADSVISWDI | GCGCCCTGGAGACTCATCGCTTGGTGGCTTTCCTGGAA | |
| | QDEKNVTCQLTF | CGCGCTGACAGCGTCATCTCTTGGGATATCCAAGATGA | |
| | WEASERTIRSEAE | GAAGAACGTGACCTGCCAGCTGACCTTCTGGGAGGCC | |
| | DSYHFSSAKMTAT | AGCGAGAGGACAATCAGAAGCGAAGCCGAGGACTCTT | |
| | FLSKKQEVNMSDS | ACCATTTCAGTTCAGCTAAGATGACCGCCACCTTCCTG | |
| | ALDCVRDEAINKL | AGCAAGAAGCAGGAAGTGAATATGTCCGATTCCGCTC | |
| | QQIFNTSYNQTYE | TTGACTGCGTCAGGGACGAAGCCATCAACAAGCTCCA | |
| | KYGNVSVFETTGG | GCAGATTTTCAATACTTCTTATAATCAGACCTATGAGA | |
| | LVVFWQGIKQKSL | AGTACGGCAATGTCAGCGTCTTCGAGACGACCGGCGG | |
| | VELERLANRSSLN | CCTGGTTGTGTTCTGGCAAGGAATCAAGCAGAAGTCA | |
| | LTHNRTKRSTDGN | CTGGTGGAGCTTGAGCGGCTGGCCAACAGATCCAGCT | |
| | NATHLSNMESVH | TGAACCTGACCCATAATCGCACCAAGCGGAGTACCGA | |
| | NLVYAQLQFTYDT | TGGCAACAACGCCACACACCTCAGCAATATGGAAAGC | |
| | LRGYINRALAQIA | GTGCACAACCTTGTGTACGCTCAGCTGCAGTTCACCTA | |
| | EAWCVDQRRTLE | CGATACCCTTAGAGGCTACATCAACAGAGCCCTGGCC | |
| | VFKELSKINPSAIL | CAGATCGCAGAGGCATGGTGCGTGGACCAGCGGCGGA | |
| | SAIYNKPIAARFM | CCCTGGAGGTGTTCAAGGAGCTCTCCAAGATCAACCC | |
| | GDVLGLASCVTIN | ATCCGCCATTCTCTCCGCCATCTACAACAAGCCTATTG | |
| | QTSVKVLRDMNV | CCGCTCGGTTCATGGGCGATGTGCTGGGACTGGCCAG | |
| | KESPGRCYSRPVVI | CTGCGTGACCATCAACCAAACCTCAGTGAAGGTGCTC | |
| | FNFANSSYVQYGQ | AGAGACATGAACGTCAAGGAGTCTCCAGGCAGATGTT | |
| | LGEDNEILLGNHR | ACAGCAGACCTGTGGTGATCTTCAACTTCGCCAATTCT | |
| | TEECQLPSLKIFIA | TCCTACGTGCAGTACGGCCAACTGGGTGAAGACAACG | |
| | GNSAYEYVDYLF | AGATTCTGTTAGGCAACCACAGGACTGAGGAGTGTCA | |
| | KRMIDLSSISTVDS | GCTGCCGAGCCTGAAGATCTTCATCGCTGGAAACAGC | |
| | MIALDIDPLENTDF | GCATACGAGTACGTGGACTACCTCTTCAAGAGGATGA | |
| | RVLELYSQKELRS | TCGACTTGTCATCTATCTCCACGGTTGATTCCATGATC | |
| | SNVFDLEEIMREF | GCCTTGGACATCGATCCTCTGGAGAATACCGACTTCAG | |
| | NSYKQHHHHHH | AGTGCTGGAGCTCTACAGCCAGAAGGAGCTTAGGTCT | |
| | (SEQ ID NO: 83) | AGCAATGTGTTCGACCTGGAGGAGATCATGAGGGAGT | |
| | | TCAATAGCTATAAGCAGCATCACCACCACCATCAC | |
| | | (SEQ ID NO: 125) | |
| SE_CMV_ | MESRIWCLVVCV | ATGGAGAGCAGAATCTGGTGCCTCGTGGTGTGCGTGA | C2/ |
| TrgB6XHis054 | NLCIVCLGAAVSS | ACCTCTGTATCGTCTGCTTAGGAGCCGCCGTGAGCAGT | CAP1/ |
| | SSTRGTSATHSHH | AGCTCTACCAGAGGCACATCCGCCACCCACAGCCACC | T100 |
| | SSHTTSAAHSRSG | ACTCTTCACACACCACCAGCGCCGCCCACTCCAGATCA | |
| | SVSQRVTSSQTVS | GGCAGCGTATCCCAGAGAGTGACCAGCAGCCAGACCG | |
| | HGVNETIYNTTLK | TGTCACATGGAGTGAATGAAACAATTTACAACACCAC | |
| | YGDVVGVNTTKY | CCTCAAGTACGGCGACGTAGTGGGAGTGAACACTACT | |
| | PYRVCSMAQGTD | AAGTACCCATACCGCGTGTGTAGCATGGCCCAGGGCA | |
| | LIRFERNIVCTSMK | CCGATCTGATCCGATTCGAGAGAAACATCGTTTGCACC | |
| | PINEDLDEGIMVV | AGCATGAAGCCTATCAACGAGGACCTGGATGAGGGCA | |
| | YKRNIVAHTFKVR | TCATGGTGGTGTACAAGAGGAACATCGTGGCCCACAC | |
| | VYQKVLTFRRSYA | GTTCAAGGTTAGGGTGTACCAGAAGGTGCTGACTTTCC | |
| | YIHTTYLLGSNTE | GAAGAAGCTATGCCTACATTCACACTACATACCTGCTC | |
| | YVAPPMWEIHHIN | GGCAGTAACACCGAGTACGTGGCGCCACCGATGTGGG | |
| | SHSQCYSSYSRVIA | AAATACACCATATTAATTCTCATAGTCAGTGCTATTCC | |
| | GTVFVAYHRDSYE | AGCTACAGCAGGGTGATCGCCGGAACCGTTTTCGTGG | |
| | NKTMQLMPDDYS | CTTATCATAGAGATTCCTACGAGAACAAGACCATGCA | |
| | NTHSTRYVTVKD | GCTGATGCCAGACGACTATAGCAACACGCATAGCACC | |
| | QWHSRGSTWLYR | CGCTACGTGACCGTGAAGGACCAGTGGCATTCAAGAG | |
| | ETCNLNCMVTITT | GATCCACCTGGCTCTACAGAGAGACATGCAATCTGAA | |
| | ARSKYPYHFFATS | CTGCATGGTGACCATCACCACCGCCCGGTCCAAGTACC | |
| | TGDVVDISPFYNG | CTTATCACTTCTTCGCCACAAGCACCGGCGATGTGGTG | |
| | TNRNASYFGENAD | GACATTTCCCCATTCTACAACGGAACCAACCGGAACG | |
| | KFTFPNYTIVSDF | CCTCTTACTTCGGCGAGAACGCCGACAAGTTCTTCATC | |
| | GRPNSALETHRLV | TTCCCAAATTATACCATCGTGAGCGACTTCGGAAGACC | |
| | AFLERADSVISWDI | TAACAGCGCCCTGGAGACACACAGACTGGTGGCCTTC | |
| | QDEKNVTCQLTF | CTCGAGCGCGCCGACTCCGTGATCTCCTGGGACATCCA | |
| | WEASERTIRSEAE | GGACGAGAAGAACGTGACTTGTCAGCTGACATTCTGG | |
| | DSYHFSSAKMTAT | GAGGCCAGCGAACGGACCATCAGAAGCGAGGCTGAA | |
| | FLSKKQEVNMSDS | GACTCCTACCACTTCAGCTCCGCCAAGATGACCGCCAC | |
| | ALDCVRDEAINKL | TTTCCTGTCAAAGAAGCAGGAGGTGAACATGAGCGAC | |
| | QQIFNTSYNQTYE | AGCGCCTTGGATTGCGTGAGAGATGAGGCCATCAACA | |
| | KYGNVSVFETTGG | AGCTTCAACAGATCTTCAACACATCCTACAACCAGAC | |
| | LVVFWQGIKQKSL | GTACGAGAAGTACGGAAACGTGAGCGTGTTCGAAACC | |
| | VELERLANRSSLN | ACCGGCGGCTTAGTGGTGTTCTGGCAGGGAATCAAGC | |
| | LTHNRTKRSTDGN | AGAAGTCTCTGGTGGAGCTGGAGAGACTGGCTAACAG | |
| | NATHLSNMESVH | ATCCTCTCTAAACCTGACACACAACAGAACCAAGCGG | |
| | NLVYAQLQFTYDT | AGCACAGACGGCAATAATGCCACACACCTGAGCAACA | |
| | LRGYINRALAQIA | TGGAAAGCGTCCACAACCTCGTCTATGCCCAACTGCA | |
| | EAWCVDQRRTLE | GTTCACCTACGACACCCTCCGAGGCTACATCAACAGA | |
| | VFKELSKINPSAIL | GCCCTGGCCCAGATCGCCGAGGCTTGGTGTGTGGATC | |
| | SAIYNKPIAARFM | AGAGACGGACCCTGGAGGTGTTCAAGGAGTTGAGCAA | |
| | GDVLGLASCVTIN | GATCAACCCGTCCCCCATCTTGACCGCTATATACAACA | |
| | QTSVKVLRDMNV | AGCCAATTGCTGCGCGGTTCATGGGCGACGTGCTGGG | |
| | KESPGRCYSRPVVI | CCTCGCCTCATGTGTGACCATTAATCAAACAAGCGTCA | |
| | FNFANSSYVQYGQ | AGGTCCTGAGGGATATGAACGTTAAGGAGAGCCCAGG | |
| | LGEDNEILLGNHR | CAGGTGCTATAGCAGACCTGTGGTGATTTTCAACTTCG | |
| | TEECQLPSLKIFIA | CCAACAGCAGCTACGTGCAGTACGGCCAGCTGGGCGA | |
| | GNSAYEYVDYLF | GGACAACGAGATCCTGCTGGGCAACCACCGCACTGAG | |
| | KRMIDLSSISTVDS | GAGTGCCAGCTGCCAAGTCTGAAGATATTCATCGCGG | |
| | MIALDIDPLENTDF | GAAATTCAGCTTACGAGTATGTAGACTACCTGTTCAAG | |
| | RVLELYSQKELRS | AGAATGATAGATCTTAGCAGCATCTCCACTGTGGACA | |
| | SNVFDLEEIMREF | GTATGATAGCTCTTGATATTGACCCACTGGAGAATACC | |
| | NSYKQHHHHHH | GACTTCAGAGTGTTGGAGCTGTACAGTCAGAAGGAGC | |
| | (SEQ ID NO: 83) | TCAGGAGCTCCAATGTGTTCGACCTGGAGGAGATCAT | |
| | | GAGGGAATTCAATAGCTACAAGCAGCACCACCACCAT | |
| | | CATCAC (SEQ ID NO: 126) | |
| SE_CMV_ | MESRIWCLVVCV | ATGGAATCCCGAATCTGGTGCCTCGTTGTGTGCGTGAA | C2/ |
| TrgB6XHis_055 | NLCIVCLGAAVSS | TTTGTGCATCGTGTGTTTGGGCGCCGCCGTCTCTTCTTC | CAP1/ |
| | SSTRGTSATHSHH | CTCCACACGTGGTACCAGCGCAACACACTCCCACCACT | T100 |
| | SSHTTSAAHSRSG | CAAGCCACACCACGTCCGCGGCACACAGCAGAAGCGG | |
| | SVSQRVTSSQTVS | CAGTGTGAGCCAAAGGGTGACCAGCAGCCAGACCGTG | |
| | HGVNETIYNTTLK | AGTCACGGCGTGAACGAGACAATCTACAATACCACAC | |
| | YGDVVGVNTTKY | TCAAGTACGGCGATGTGGTGGGCGTCAACACCACCAA | |
| | PYRVCSMAQGTD | GTATCCTTACAGAGTCTGTTCCATGGCCCAGGGCACTG | |
| | LIRFERNIVCTSMK | ACCTGATCCGGTTCGAAAGAAACATAGTGTGCACCTC | |
| | PINEDLDEGIMVV | CATGAAGCCTATCAATGAGGACCTCGATGAAGGCATT | |
| | YKRNIVAHTFKVR | ATGGTGGTGTACAAGAGGAATATTGTGGCCCATACCTT | |
| | VYQKVLTFRRSYA | CAAGGTGAGAGTGTACCAGAAGGTGCTGACCTTCAGA | |
| | YIHTTYLLGSNTE | CGGAGCTACGCCTACATCCATACAACCTACCTGCTGGG | |
| | YVAPPMWEIHHIN | AAGCAACACCGAGTACGTGGCTCCTCCAATGTGGGAG | |
| | SHSQCYSSYSRVIA | ATCCACCACATCAATAGCCACAGCCAGTGCTACTCCA | |
| | GTVFVAYHRDSYE | GCTACAGCAGAGTGATTGCTGGCACCGTCTTCGTGGCT | |
| | NKTMQLMPDDYS | TACCACAGAGACAGCTATGAGAACAAGACAATGCAGC | |
| | NTHSTRYVTVKD | TCATGCCAGACGACTACTCTAACACACATTCAACCCGG | |
| | QWHSRGSTWLYR | TATGTGACCGTGAAGGACCAGTGGCACTCAAGAGGCA | |
| | ETCNLNCMVTITT | GCACATGGCTCTACCGAGAGACATGTAACCTGAACTG | |
| | ARSKYPYHFFATS | CATGGTTACAATCACCACTGCAAGGTCTAAGTACCCAT | |
| | TGDVVDISPFYNG | ATCACTTCTTCGCCACCTCTACCGGACACGTGGTGGAC | |
| | TNRNASYFGENAD | ATCAGCCCATTCTACAATGGCACCAATCGGAACGCAA | |
| | KFFIFPNYTIVSDF | GCTACTTCGGAGAGAACGCCGACAAGTTCTTCATCTTC | |
| | GRPNSALETHRLV | CCGAACTACACCATCGTGTCCGATTTCGGCAGGCCAA | |
| | AFLERADSVISWDI | ACAGCGCCCTGGAGACACACCGGCTGGTGGCCTTCCT | |
| | QDEKNVTCQLTF | GGAGCGCGCTGACTCCGTTATCTCTTGGGACATCCAGG | |
| | WEASERTIRSEAE | ATGAGAAGAATGTGACCTGCCAACTGACATTCTGGGA | |
| | DSYHFSSAKMTAT | GGCATCCGAGCGGACTATCAGAAGCGAGGCCGAGGAC | |
| | FLSKKQEVNMSDS | AGCTACCACTTCAGCAGCGCTAAGATGACTGCTACCTT | |
| | ALDCVRDEAINKL | CCTGTCCAAGAAGCAGGAGGTGAACATGTCTGATTCC | |
| | QQIFNTSYNQTYE | GCTCTGGACTGCGTGAGGGACGAGGCTATCAACAAGC | |
| | KYGNVSVFETTGG | TCCAGCAGATATTCAATACTTCCTACAACCAGACCTAC | |
| | LVVFWQGIKQKSL | GAGAAGTACGGTAACGTCAGCGTTTTCGAAACCACCG | |
| | VELERLANRSSLN | GCGGCCTGGTCGTGTTCTGGCAGGGAATCAAGCAGAA | |
| | LTHNRTKRSTDGN | GTCCCTTGTCGAGCTCGAGAGACTGGCCAACCGGTCTA | |
| | NATHLSNMESVH | GCCTCAATCTGACACACAATAGGACCAAGAGATCTAC | |
| | NLVYAQLQFTYDT | TGACGGCAATAACGCCACACACCTCTCCAACATGGAG | |
| | LRGYINRALAQIA | AGTGTTCATAACCTGGTTTACGCCCAGCTGCAGTTCAC | |
| | EAWCVDQRRTLE | TTACGATACCCTCCGCGGCTACATCAACAGGGCCCTGG | |
| | VFKELSKINPSAIL | CGCAGATCGCCGAGGCCTGGTGCGTGGATCAAAGAAG | |
| | SAIYNKPIAARFM | GACCCTGGAGGTCTTCAAGGAACTCAGCAAGATCAAC | |
| | GDVLGLASCVTIN | CCATCTGCTATCCTGAGCGCCATCTACAACAAGCCAAT | |
| | QTSVKVLRDMNV | CGCCGCCCGGTTCATGGGCGACGTCCTGGGCTTGGCTA | |
| | KESPGRCYSRPVVI | GCTGCGTGACCATCAATCAGACCAGCGTCAAGGTGCT | |
| | FNFANSSYVQYGQ | TCGCGACATGAACGTCAAGGAGTCACCTGGCCGCTGT | |
| | LGEDNEILLGNHR | TACTCAAGGCCAGTCGTGATCTTCAATTTCGCCAATAG | |
| | TEECQLPSLKIFIA | CTCCTACGTGCAGTACGGACAGTTGGGCGAGGACAAT | |
| | GNSAYEYVDYLF | GAAATACTCCTGGGCAACCACCGCACCGAGGAGTGTC | |
| | KRMIDLSSISTVDS | AGCTGCCAAGCCTGAAGATCTTCATCGCGGGAAACTC | |
| | MIALDIDPLENTDF | CGCTTACGAGTATGTGGACTACCTGTTCAAGAGAATG | |
| | RVLELYSQKELRS | ATTGATCTGAGCAGCATCTCCACCGTGGACAGCATGAT | |
| | SNVTDIEEIMREF | TGCTCTGGATATTGATCCTCTGGAGAACACCGATTTCC | |
| | NSYKQHHHHHH | GCGTGCTGGAGCTGTACAGCCAGAAGGAATTAAGGAG | |
| | (SEQ ID NO: 83) | CAGTAATGTGTTCGACCTGGAGGAGATCATGAGGGAG | |
| | | TTCAACAGTTACAAGCAGCACCACCATCACCACCAC | |
| | | (SEQ ID NO: 127) | |
| SE_CMV_ | MESRIWCLVVCV | ATGGAGTCCAGAATCTGGTGCTTGGTGGTGTGCGTGA | C2/ |
| TrgBXHis_056 | NLCIVCLGAAVSS | ATCTTTGCATTGTGTGCCTCGGCGCCGCCGTGAGCAGC | CAP1/ |
| | SSTRGTSATHSHH | AGCAGTACTAGAGGTACCTCCGCTACCCACAGCCACC | TI00 |
| | SSHTTSAAHSRSG | ACTCTTCCCATACAACCAGCGCCGCCCACTCACGTAGC | |
| | SVSQRVTSSQTVS | GGCTCTGTGAGCCAGAGGGTGACAAGCTCACAGACCG | |
| | HGVNETIYNTTLK | TGAGCCACGGCGTGAACGAGACTATCTACAACACTAC | |
| | YGDVVGVNTTKY | CCTGAAGTACGGCGATGTGGTGGGAGTGAATACCACA | |
| | PYRVCSMAQGTD | AAGTACCCGTACAGGGTGTGTTCCATGGCCCAGGGCA | |
| | LIRFERNIVCTSMK | CCGACCTGATTCGCTTCGAAAGAAACATCGTCTGCACC | |
| | PINEDLDEGIMVV | AGCATGAAGCCTATCAACGAGGATTTGGATGAGGGTA | |
| | YKRNIVAHTFKVR | TTATGGTGGTCTACAAGAGAAATATTGTGGCCCACACC | |
| | VYQKVLTFRRSYA | TTCAAGGTCAGAGTGTACCAGAAGGTCCTGACGTTCA | |
| | YIHTTYLLGSNTE | GGAGATCTTACGCTTACATCCACACCACCTACCTTCTG | |
| | YVAPPMWEIHHIN | GGCTCCAACACCGAGTATGTGGCCCCGCCTATGTGGG | |
| | SHSQCYSSYSRVIA | AGATCCACCACATTAATTCCCACTCTCAATGCTACAGC | |
| | GTVFVAYHRDSYE | TCCTATTCCAGAGTGATCGCCGGCACAGTCTTCGTGGC | |
| | NKTMQLMPDDYS | CTACCACCGGGACAGCTATGAGAACAAGACTATGCAG | |
| | NTHSTRYVTVKD | CTCATGCCAGACGACTATAGCAATACTCATAGCACTA | |
| | QWHSRGSTWLYR | GATATGTGACTGTGAAGGACCAGTGGCATAGCAGAGG | |
| | ETCNLNCMVTITT | CAGCACTTGGCTGTACCGGGAAACATGCAATCTTAATT | |
| | ARSKYPYHFFATS | GCATGGTCACCATAACCACCGCGAGATCCAAGTACCC | |
| | TGDVVDISPFYNG | TTACCACTTCTTCGCCACCTCCACTGGTGACGTCGTGG | |
| | TNRNASYFGENAD | ACATCTCCCCTTTCTATAACGGAACAAATAGAAACGCC | |
| | KFFIFPNYTIVSDF | AGCTACTTCGGTGAGAACGCCGACAAGTTCTTCATCTT | |
| | GRPNSALETHRLV | CCCTAACTACACCATAGTGAGCGATTTCGGCAGACCG | |
| | AFLERADSVISWDI | AACTCCGCTCTGGAGACACACCGGCTGGTGGCCTTCCT | |
| | QDEKNVTCQLTF | GGAACGGGCCGATAGTGTTATCTCTTGGGATATTCAAG | |
| | WEASERTIRSEAE | ACGAGAAGAACGTCACCTGTCAGCTGACTTTCTGGGA | |
| | DSYHFSSAKMTAT | AGCCAGCGAGAGGACCATCAGAAGTGAAGCTGAGGAT | |
| | FLSKKQEVNMSDS | AGCTACCATTTCTCTAGTGCCAAGATGACTGCCACCTT | |
| | ALDCVRDEAINKL | CCTGTCCAAGAAGCAGGAGGTCAACATGTCCGACAGC | |
| | QQIFNTSYNQTYE | GCCCTCGACTGTGTGAGAGACGAGGCTATTAACAAGC | |
| | KYGNVSVFETTGG | TGCAGCAGATTTTCAACACTAGCTACAATCAGACATAC | |
| | LVVFWQGIKQKSL | GAGAAGTATGGAAACGTGAGCGTGTTCGAAACTACCG | |
| | VELERLANRSSLN | GTGGCCTGGTGGTATTCTGGCAGGGCATCAAGCAGAA | |
| | LTHNRTKRSTDGN | GTCCCTGGTGGAATTGGAGAGACTGGCTAACAGGTCG | |
| | NATHLSNMESVH | TCCCTGAACCTGACTCACAATAGAACGAAGAGGAGCA | |
| | NLVYAQLQFTYDT | CAGACGGCAATAATGCCACCCATCTGTCCAATATGGA | |
| | LRGYINRALAQIA | GAGTGTGCACAATTTGGTGTATGCCCAGCTGCAGTTCA | |
| | EAWCVDQRRTLE | CCTACGACACCCTCAGGGGCTACATCAACAGAGCCCT | |
| | VFKELSKINPSAIL | CGCCCAGATCGCTGAAGCCTGGTGCGTGGATCAGAGG | |
| | SAIYNKPIAARFM | AGGACCCTGGAGGTCTTCAAGGAACTGAGCAAGATAA | |
| | GDVLGLASCVTIN | ACCCATCCGCCATCCTCAGTGCCATTTATAACAAGCCT | |
| | QTSVKVLRDMNV | ATTGCCGCCAGGTTCATGGGCGACGTGCTGGGCCTGG | |
| | KESPGRCYSRPVVI | CTTCCTGTGTCACGATTAATCAGACCTCCGTGAAGGTG | |
| | FNFANSSYVQYGQ | CTGAGGGACATGAACGTGAAGGAAAGCCCTGGACGGT | |
| | LGEDNEILLGNHR | GTTACAGCCGACCAGTAGTGATCTTCAACTTCGCCAAC | |
| | TEECQLPSLKIFIA | TCCTCATACGTGCAGTATGGCCAGCTGGGCGAGGACA | |
| | GNSAYEYVDYLF | ATGAAATTCTGCTGGGCAACCACAGGACCGAAGAGTG | |
| | KRMIDLSSISTVDS | CCAGCTGCCTAGCCTGAAGATATTCATCGCCGGTAATA | |
| | MIALDIDPLENTDF | GCGCCTACGAGTACGTCGACTATCTTTTCAAGAGAATG | |
| | RVLELYSQKELRS | ATCGATCTGTCTAGCATTTCTACCGTGGATTCCATGAT | |
| | SNVFDLEEIMREF | CGCTCTTGACATTGACCCACTGGAGAACACAGACTTCA | |
| | NSYKQHHHHHH | GGGTGCTCGAGCTGTATTCCCAGAAGGAACTCAGGTC | |
| | (SEQ ID NO: 83) | TAGCAACGTTTTCGACCTCGAGGAAATTATGAGAGAG | |
| | | TTCAACTCGTACAAGCAACACCATCACCACCATCAC | |
| | | (SEQ ID NO: 128) | |
| SE_CMV_ | MESRIWCLVVCV | ATGGAGAGCCGGATCTGGTGCCTCGTGGTGTGCGTGA | C2/ |
| TrgB6XHis_057 | NLCIVCLGAAVSS | ACTTATGCATCGTGTGCCTCGGCGCCGCCGTGAGCTCT | CAP1/ |
| | SSTRGTSATHSHH | AGCTCTACCCGGGGCACCAGCGCCACCCACAGCCACC | T100 |
| | SSHTTSAAHSRSG | ACAGCAGCCACACCACCTCGGCCGCTCACAGCCGGAG | |
| | SVSQRVTSSQTVS | CGGCAGCGTGAGCCAGCGGGTGACCTCCAGCCAGACC | |
| | HGVNETIYNTTLK | GTGTCCCACGGCGTGAACGAGACGATCTACAACACCA | |
| | YGDVVGVNTTKY | CCCTGAAGTACGGCGACGTGGTGGGAGTGAACACGAC | |
| | PYRVCSMAQGTD | CAAGTACCCCTACCGGGTGTGCAGCATGGCCCAGGGC | |
| | LIRFERNIVCTSMK | ACCGACCTGATCCGGTTCGAGCGGAACATTGTGTGCA | |
| | PINEDLDEGIMVV | CCAGCATGAAGCCCATCAACGAGGACCTGGACGAGGG | |
| | YKRNIVAHTFKVR | CATCATGGTAGTGTACAAGAGAAACATCGTGGCCCAC | |
| | VYQKVLTFRRSYA | ACCTTCAAGGTGCGGGTGTACCAGAAGGTGCTGACCT | |
| | YIHTTYLLGSNTE | TCCGGCGGAGCTACGCCTACATTCACACAACATACCTG | |
| | YVAPPMWEIHHIN | CTGGGCAGCAACACCGAGTACGTGGCTCCTCCCATGT | |
| | SHSQCYSSYSRVIA | GGGAGATCCACCACATCAACTCTCATAGCCAGTGCTA | |
| | GTVFVAYHRDSYE | CAGCAGCTACAGCCGGGTGATCGCCGGCACCGTGTTC | |
| | NKTMQLMPDDYS | GTGGCCTACCACCGGGACAGCTACGAGAACAAGACCA | |
| | NTHSTRYVTVKD | TGCAGCTGATGCCCGACGACTATAGCAACACACACTC | |
| | QWHSRGSTWLYR | CACTCGGTACGTGACCGTGAAGGACCAGTGGCACAGC | |
| | ETCNLNCMVTITT | AGAGGCAGCACCTGGCTGTACCGGGAGACTTGCAACC | |
| | ARSKYPYHFFATS | TGAACTGCATGGTGACCATCACCACCGCCCGGTCTAA | |
| | TGDVVDISPFYNG | GTACCCTTACCACTTCTTCGCCACCAGCACCGGCGATG | |
| | TNRNASYFGENAD | TGGTGGACATCAGCCCCTTCTACAACGGCACCAACCG | |
| | KFFIFPNYTIVSDF | GAACGCCAGCTACTTCGGCGAGAACGCCGACAAGTTC | |
| | GRPNSALETHRLV | TTCATCTTCCCCAACTACACCATCGTGAGCGACTTCGG | |
| | AFLERADSVISWDI | CCGGCCCAACAGCGCCCTGGAAACTCACCGGCTGGTG | |
| | QDEKNVTCQLTF | GCCTTCCTGGAGCGGGCCGACAGCGTGATCAGCTGGG | |
| | WEASERTIRSEAE | ACATCCAGGACGAGAAGAACGTGACCTGCCAGCTGAC | |
| | DSYHFSSAKMTAT | ATTCTGGGAGGCCAGCGAGCGGACCATCCGGAGCGAG | |
| | FLSKKQEVNMSDS | GCCGAGGATAGCTATCACTTCAGCAGCGCCAAGATGA | |
| | ALDCVRDEAINKL | CCGCCACCTTCCTGAGCAAGAAGCAGGAGGTGAACAT | |
| | QQIFNTSYNQTYE | GAGCGATTCTGCACTGGACTGCGTGCGGGACGAGGCC | |
| | KYGNVSVFETTGG | ATCAACAAGCTGCAGCAGATCTTCAACACCAGCTACA | |
| | LVVFWQGIKQKSL | ACCAGACCTACGAGAAGTACGGAAACGTGAGCGTGTT | |
| | VELERLANRSSLN | CGAGACTACCGGCGGCCTTGTCGTGTTCTGGCAGGGA | |
| | LTHNRTKRSTDGN | ATCAAGCAGAAGTCCCTGGTCGAGCTCGAGCGACTGG | |
| | NATHLSNMESVH | CCAACAGAAGCAGCCTGAACCTGACCCACAACCGGAC | |
| | NLVYAQLQFTYDT | CAAGCGGAGCACCGACGGCAACAACGCCACACACCTG | |
| | LRGYINRALAQIA | TCTAACATGGAGTCTGTGCACAACCTGGTGTACGCCCA | |
| | EAWCVDQRRTLE | GCTGCAGTTCACCTACGACACCCTGCGGGGCTACATCA | |
| | VFKELSKINPSAIL | ACCGGGCCCTGGCCCAGATCGCCGAGGCATGGTGCGT | |
| | SAIYNKPIAARFM | GGACCAGCGGCGGACCCTGGAGGTGTTCAAGGAGCTC | |
| | GDVLGLASCVTIN | TCTAAGATCAACCCGTCTGCCATCCTGAGCGCCATTTA | |
| | QTSVKVLRDMNV | CAACAAGCCTATCGCCGCAAGATTCATGGGCGACGTC | |
| | KESPGRCYSRPVVI | CTGGGCCTGGCCAGCTGCGTGACGATCAATCAGACCA | |
| | FNFANSSYVQYGQ | GCGTGAAGGTCCTGCGGGACATGAACGTCAAGGAGAG | |
| | LGEDNEILLGNHR | CCCCGGCAGGTGCTATAGCCGGCCCGTGGTGATTTTCA | |
| | TEECQLPSLKIFIA | ACTTCGCCAATAGCTCTTACGTGCAGTACGGTCAGTTA | |
| | GNSAYEYVDYLF | GGCGAGGACAACGAGATCTTACTGGGCAACCACCGGA | |
| | KRMIDLSSISTVDS | CCGAGGAGTGCCAACTCCCGAGCCTCAAGATTTTCATT | |
| | MIALDIDPLENTDF | GCCGGCAATAGCGCATACGAATATGTGGACTACCTGT | |
| | RVLELYSQKELRS | TCAAGCGGATGATCGACCTGAGCAGCATCAGCACCGT | |
| | SNVFDLEEIMREF | GGACAGCATGATTGCTCTGGACATCGACCCTCTGGAG | |
| | NSYKQHHHHHH | AACACCGACTTCCGGGTGCTGGAGCTGTACAGCCAGA | |
| | (SEQ ID NO: 83) | AGGAGCTGCGGAGCTCTAATGTGTTCGACCTGGAGGA | |
| | | GATCATGCGGGAGTTCAACTCATATAAGCAGCACCAC | |
| | | CACCATCATCAC (SEQ ID NO: 129) | |
| SE_CMV_ | MESRIWCLVVCV | ATGGAGAGCCGGATCTGGTGCCTCGTGGTGTGCGTGA | C2/ |
| TrgB6XHis_058 | NLCIVCLGAAVSS | ACCTTTGCATCGTGTGCTTGGGCGCCGCCGTGAGCAGC | CAP1/ |
| | SSTRGTSATHSHH | AGCTCCACCCGGGGCACCTCCGCCACCCACTCCCACCA | T100 |
| | SSHTTSAAHSRSG | CTCCTCCCACACCACCTCAGCCGCCCACTCTCGCTCCG | |
| | SVSQRVTSSQTVS | GCTCCGTCTCCCAGCGCGTCACCTCCAGCCAGACCGTT | |
| | HGVNETIYNTTLK | AGCCACGGCGTCAACGAAACCATCTACAACACCACCC | |
| | YGDVVGVNTTKY | TCAAGTACGGCGACGTCGTCGGCGTAAACACAACCAA | |
| | PYRVCSMAQGTD | GTACCCCTACCGCGTCTGCTCCATGGCCCAGGGCACCG | |
| | LIRFERNIVCTSMK | ACCTCATCCGCTTCGAGCGCAACATCGTCTGCACCTCC | |
| | PINEDLDEGIMVV | ATGAAGCCCATCAACGAGGACCTCGACGAGGGCATCA | |
| | YKRNIVAHTFKVR | TGGTCGTCTACAAGCGCAATATTGTGGCCCACACCTTC | |
| | VYQKVLTFRRSYA | AAGGTCCGCGTCTACCAGAAGGTCCTCACCTTCCGCCG | |
| | YIHTTYLLGSNTE | CTCCTACGCCTACATCCACACAACCTACCTCCTCGGCT | |
| | YVAPPMWEIHHIN | CCAACACCGAGTACGTCGCCCCTCCCATGTGGGAGAT | |
| | SHSQCYSSYSRVIA | CCACCACATCAACAGCCACAGCCAGTGCTACTCCTCCT | |
| | GTVFVAYHRDSYE | ACTCCCGCGTCATCGCCGGCACCGTCTTCGTCGCCTAC | |
| | NKTMQLMPDDYS | CACCGCGACTCCTACGAGAACAAGACCATGCAGCTCA | |
| | NTHSTRYVTVKD | TGCCCGACGACTACAGCAATACCCACAGCACCCGCTA | |
| | QWHSRGSTWLYR | CGTCACCGTCAAGGACCAGTGGCACAGCAGAGGCTCC | |
| | ETCNLNCMVTITT | ACCTGGCTCTACCGCGAGACATGCAACCTCAACTGCAT | |
| | ARSKYPYHFFATS | GGTCACCATCACCACCGCCCGCTCCAAGTATCCTTACC | |
| | TGDVVDISPFYNG | ACTTCTTCGCCACCTCCACCGGCGATGTCGTGGACATC | |
| | TNRNASYFGENAD | TCACCATTCTACAACGGCACCAACCGCAACGCCAGTT | |
| | KFFIFPNYTIVSDF | ACTTCGGCGAGAACGCCGACAAGTTCTTCATCTTCCCC | |
| | GRPNSALETHRLV | AACTACACCATCGTCTCCGACTTCGGCCGCCCCAACTC | |
| | AFLERADSVISWDI | CGCCCTCGAGACACACAGACTGGTGGCCTTCCTCGAG | |
| | QDEKNVTCQLTF | CGCGCCGACTCCGTCATCTCCTGGGACATCCAGGACG | |
| | WEASERTIRSEAE | AGAAGAACGTCACCTGCCAGCTCACATTCTGGGAGGC | |
| | DSYHFSSAKMTAT | CTCCGAGCGCACCATCCGCTCCGAGGCCGAGGACTCA | |
| | FLSKKQEVNMSDS | TACCATTTCTCCAGCGCCAAGATGACCGCCACCTTCCT | |
| | ALDCVRDEAINKL | CTCCAAGAAGCAGGAGGTCAACATGAGCGACAGCGCT | |
| | QQIFNTSYNQTYE | CTCGACTGCGTCCGCGACGAGGCCATCAACAAGCTCC | |
| | KYGNVSVFETTGG | AGCAGATCTTCAACACCTCCTACAACCAGACGTACGA | |
| | LVVFWQGIKQKSL | GAAGTATGGAAACGTCAGTGTCTTCGAAACCACGGGC | |
| | VELERLANRSSLN | GGCCTGGTTGTATTCTGGCAGGGAATAAAGCAGAAGT | |
| | LTHNRTKRSTDGN | CCCTCGTCGAGCTTGAGCGCCTCGCCAACCGCTCCTCC | |
| | NATHLSNMESVH | CTCAACCTCACCCACAACCGCACCAAGCGCTCCACCG | |
| | NLVYAQLQFTYDT | ACGGCAACAACGCTACCCACCTGTCCAACATGGAGTC | |
| | LRGYINRALAQIA | CGTCCACAACCTCGTCTACGCCCAGCTCCAGTTCACCT | |
| | EAWCVDQRRTLE | ACGACACCCTCCGCGGCTACATCAACCGCGCCCTCGCC | |
| | VFKELSKINPSAIL | CAGATCGCCGAGGCCTGGTGCGTCGACCAGCGCCGCA | |
| | SAIYNKPIAARFM | CCCTCGAGGTCTTCAAGGAGCTGAGTAAGATCAACCC | |
| | GDVLGLASCVTIN | TAGCGCGATCCTCAGCGCTATCTATAACAAGCCAATCG | |
| | QTSVKVLRDMNV | CTGCTAGGTTCATGGGAGACGTGCTCGGCCTCGCCTCC | |
| | KESPGRCYSRPVVI | TGCGTGACCATCAATCAGACATCCGTGAAGGTGCTGC | |
| | FNFANSSYVQYGQ | GCGACATGAATGTCAAGGAGAGCCCAGGCCGCTGTTA | |
| | LGEDNEILLGNHR | TTCCCGGCCCGTCGTCATTTTCAATTTCGCCAATAGCT | |
| | TEECQLPSLKIFIA | CTTACGTCCAGTACGGCCAGCTCGGCGAGGACAACGA | |
| | GNSAYEYVDYLF | GATCCTGCTGGGCAACCACCGCACCGAGGAGTGCCAG | |
| | KRMIDLSSISTVDS | CTGCCTAGCCTCAAGATTTTCATTGCCGGCAATTCCGC | |
| | MIALDIDPLENTDF | TTACGAATACGTGGACTACCTCTTCAAGCGCATGATCG | |
| | RVLELYSQKELRS | ACCTCTCCTCCATCTCCACCGTCGACTCCATGATCGCC | |
| | SNVFDLEEIMREF | CTGGATATCGACCCTCTCGAGAACACCGACTTCCGCGT | |
| | NSYKQHHHHHH | GTTGGAGCTCTACTCCCAGAAGGAGCTCAGATCCAGC | |
| | (SEQ ID NO: 83) | AACGTATTCGACCTCGAGGAGATCATGCGCGAGTTCA | |
| | | ACTCCTATAAGCAGCACCACCACCATCATCAC (SEQ ID | |
| | | NO: 130) | |
| SE_CMV_ | MESRIWCLVVCV | ATGGAGAGCCGGATCTGGTGCTTGGTGGTGTGCGTGA | C2/ |
| TrgB6XHis_059 | NLCIVCLGAAVSS | ACTTGTGCATCGTGTGCTTGGGCGCCGCCGTGAGCAGC | CAP1/ |
| | SSTRGTSATHSHH | TCTAGCACCCGGGGCACCAGCGCCACCCACAGCCACC | T100 |
| | SSHTTSAAHSRSG | ACAGCAGCCACACGACCTCCGCCGCCCACTCACGGAG | |
| | SVSQRVTSSQTVS | CGGCAGCGTGAGCCAGCGGGTGACCAGCTCACAGACC | |
| | HGVNETIYNTTLK | GTGTCCCACGGCGTGAACGAGACGATCTACAACACCA | |
| | YGDVVGVNTTKY | CCCTGAAGTACGGCGACGTGGTGGGCGTCAACACTAC | |
| | PYRVCSMAQGTD | CAAGTACCCCTACCGGGTGTGCAGCATGGCCCAGGGC | |
| | LIRFERNIVCTSMK | ACCGACCTGATCCGGTTCGAGCGGAATATTGTGTGTAC | |
| | PINEDLDEGIMVV | CAGCATGAAGCCCATCAACGAGGACCTGGACGAGGGC | |
| | YKRNIVAHTFKVR | ATCATGGTGGTCTACAAGAGAAACATTGTGGCCCACA | |
| | VYQKVLTFRRSYA | CCTTCAAGGTGCGGGTGTACCAGAAGGTGCTGACCTTC | |
| | YIHTTYLLGSNTE | CGGCGGAGCTACGCCTACATCCATACAACCTACCTGCT | |
| | YVAPPMWEIHHIN | GGGCAGCAACACCGAGTACGTGGCGCCTCCCATGTGG | |
| | SHSQCYSSYSRVIA | GAGATCCACCACATCAACTCTCACTCGCAGTGCTACAG | |
| | GTVFVAYHRDSYE | CAGCTACAGCCGGGTGATCGCCGGCACCGTGTTCGTG | |
| | NKTMQLMPDDYS | GCCTACCACCGGGACAGCTACGAGAACAAGACCATGC | |
| | NTHSTRYVTVKD | AGCTGATGCCCGACGACTATTCTAACACCCACTCCACC | |
| | QWHSRGSTWLYR | AGATACGTGACCGTGAAGGACCAGTGGCACAGCAGGG | |
| | ETCNLNCMVTITT | GCAGCACCTGGCTGTACCGGGAGACTTGCAACCTGAA | |
| | ARSKYPYHFFATS | CTGCATGGTGACCATCACCACCGCCCGGAGTAAGTAT | |
| | TGDVVDISPFYNG | CCATATCACTTCTTCGCCACCAGCACGGGCGACGTTGT | |
| | TNRNASYFGENAD | GGACATCAGCCCCTTCTACAACGGCACCAACCGGAAC | |
| | KFFIFPNYTIVSDF | GCCAGCTACTTCGGCGAGAACGCCGACAAGTTCTTCAT | |
| | GRPNSALETHRLV | CTTCCCCAACTACACCATCGTGAGCGACTTCGGCCGGC | |
| | AFLERADSVISWDI | CCAACAGCGCCCTGGAAACCCACCGGCTGGTGGCCTT | |
| | QDEKNVTCQLTF | CCTGGAGCGGGCCGACAGCGTGATCAGCTGGGACATC | |
| | WEASERTIRSEAE | CAGGACGAGAAGAACGTGACCTGCCAGCTGACTTTCT | |
| | DSYHFSSAKMTAT | GGGAGGCCAGCGAGCGGACCATCCGGAGCGAGGCCG | |
| | FLSKKQEVNMSDS | AAGACTCCTACCACTTCAGCAGCGCCAAGATGACCGC | |
| | ALDCVRDEAINKL | CACCTTCCTGAGCAAGAAGCAGGAGGTGAACATGAGC | |
| | QQIFNTSYNQTYE | GATTCAGCTCTGGACTGCGTGCGGGACGAGGCCATCA | |
| | KYGNVSVFETTGG | ACAAGCTGCAGCAGATCTTCAACACCAGCTACAACCA | |
| | LVVFWQGIKQKSL | GACTTACGAGAAGTATGGAAACGTGAGCGTGTTCGAG | |
| | VELERLANRSSLN | ACAACCGGCGGCCTCGTGGTGTTCTGGCAGGGTATCA | |
| | LTHNRTKRSTDGN | AGCAGAAGTCTCTCGTGGAGCTGGAGAGACTGGCCAA | |
| | NATHLSNMESVH | CAGAAGCAGCCTGAACCTGACCCACAACCGGACCAAG | |
| | NLVYAQLQFTYDT | CGGAGCACCGACGGCAACAACGCTACCCATCTGTCTA | |
| | LRGYINRALAQIA | ACATGGAGTCAGTGCACAACCTGGTGTACGCCCAGCT | |
| | EAWCVDQRRTLE | GCAGTTCACCTACGACACCCTGCGGGGCTACATCAAC | |
| | VFKELSKINPSAIL | CGGGCCCTGGCCCAGATCGCCGAGGCCTGGTGCGTGG | |
| | SAIYNKPIAARFM | ACCAGCGGCGGACCCTGGAGGTGTTCAAGGAGCTGTC | |
| | GDVLGLASCVTIN | CAAGATCAACCCTTCCGCCATCCTGAGCGCCATTTATA | |
| | QTSVKVLRDMNV | ATAAGCCGATCGCCGCCCGGTTCATGGGCGATGTTCTG | |
| | KESPGRCYSRPVVI | GGCCTGGCCAGCTGCGTCACCATTAATCAGACCAGCG | |
| | FNFANSSYVQYGQ | TTAAGGTCCTGCGGGACATGAATGTCAAGGAGAGCCC | |
| | LGEDNEILLGNHR | CGGCAGGTGCTACTCCCGCCCCGTGGTGATATTCAACT | |
| | TEECQLPSLKIFIA | TCGCCAACTCTAGCTACGTGCAGTACGGCCAACTAGG | |
| | GNSAYEYVDYLF | CGAGGACAACGAGATCTTGCTCGGTAACCACCGGACC | |
| | KRMIDLSSISTVDS | GAGGAGTGCCAGTTACCTTCCCTGAAGATTTTCATCGC | |
| | MIALDIDPLENTDF | GGGCAACTCCGCCTACGAGTATGTGGACTACCTGTTCA | |
| | RVLELYSQKELRS | AGCGGATGATCGATCTTTCTAGCATCAGCACCGTGGAC | |
| | SNVFDLEEIMREF | AGCATGATAGCCCTGGACATCGACCCACTGGAGAACA | |
| | NSYKQHHHHHH | CCGACTTCCGGGTGCTGGAGCTGTACAGCCAGAAGGA | |
| | (SEQ ID NO: 83) | GCTTCGGAGCAGCAATGTGTTCGACCTGGAGGAGATC | |
| | | ATGCGGGAGTTCAATTCTTACAAGCAGCACCACCACC | |
| | | ATCATCAC (SEQ ID NO: 131) | |
| SE_CMV_ | MESRIWCLVVCV | ATGGAGAGCCGGATCTGGTGCCTCGTGGTGTGCGTGA | C2/ |
| TrgBXHis_060 | NLCIVCLGAAVSS | ACCTCTGCATCGTGTGCCTCGGCGCCGCCGTGTCTTCA | CAP1/ |
| | SSTRGTSATHSHH | TCCTCCACCCGGGGCACCTCCGCCACCCACTCCCACCA | T100 |
| | SSHTTSAAHSRSG | CTCCTCCCACACCACTAGTGCCGCCCACTCACGCTCCG | |
| | SVSQRVTSSQTVS | GCTCCGTCTCCCAGCGCGTCACCTCATCCCAGACAGTG | |
| | HGVNETIYNTTLK | AGCCACGGCGTCAACGAAACCATCTACAACACCACCC | |
| | YGDVVGVNTTKY | TCAAGTACGGCGACGTCGTGGGCGTGAACACTACAAA | |
| | PYRVCSMAQGTD | GTACCCCTACCGCGTCTGCTCCATGGCCCAGGGCACCG | |
| | LIRFERNIVCTSMK | ACCTGATCCGCTTCGAGCGCAACATCGTCTGCACCTCC | |
| | PINEDLDEGIMVV | ATGAAGCCCATCAACGAGGACCTCGACGAGGGCATCA | |
| | YKRNIVAHTFKVR | TGGTCGTCTACAAGAGGAACATTGTGGCCCACACCTTC | |
| | VYQKVLTFRRSYA | AAGGTCCGCGTCTACCAGAAGGTCCTCACCTTCCGCCG | |
| | YIHTTYLLGSNTE | CTCCTACGCCTACATCCACACTACGTACCTCCTCGGCT | |
| | YVAPPMWEIHHIN | CCAACACCGAGTACGTCGCCCCTCCCATGTGGGAGAT | |
| | SHSQCYSSYSRVIA | CCACCACATCAACTCGCACAGCCAGTGCTACTCCTCCT | |
| | GTVFVAYHRDSYE | ACTCCCGCGTCATCGCCGGCACCGTCTTCGTCGCCTAC | |
| | NKTMQLMPDDYS | CACCGCGACTCCTACGAGAACAAGACCATGCAGCTCA | |
| | NTHSTRYVTVKD | TGCCCGACGACTATAGCAACACACATAGCACCCGCTA | |
| | QWHSRGSTWLYR | CGTCACCGTCAAGGACCAGTGGCATAGCAGAGGCTCC | |
| | ETCNLNCMVTITT | ACCTGGCTCTACCGCGAGACGTGCAACCTCAACTGCAT | |
| | ARSKYPYHFFATS | GGTCACCATCACCACCGCCCGCAGCAAGTATCCATATC | |
| | TGDVVDISPFYNG | ACTTCTTCGCCACCTCCACAGGAGACGTGGTCGACATC | |
| | TNRNASYFGENAD | TCGCCTTTCTACAACGGCACCAACCGCAACGCTAGCTA | |
| | KFFIFPNYTIVSDF | CTTCGGCGAGAACGCCGACAAGTTCTTCATCTTCCCCA | |
| | GRPNSALETHRLV | ACTACACCATCGTCTCCGACTTCGGCCGCCCCAACTCC | |
| | AFLERADSVISWDI | GCCCTCGAGACTCACCGCTTGGTGGCCTTCCTCGAGCG | |
| | QDEKNVTCQLTF | CGCCGACTCCGTCATCTCCTGGGACATCCAGGACGAG | |
| | WEASERTIRSEAE | AAGAACGTCACCTGCCAGCTGACCTTCTGGGAGGCCT | |
| | DSYHFSSAKMTAT | CCGAGCGCACCATCCGCTCCGAGGCCGAGGACAGCTA | |
| | FLSKKQEVNMSDS | CCACTTCAGCAGCGCCAAGATGACCGCCACCTTCCTCT | |
| | ALDCVRDEAINKL | CCAAGAAGCAGGAGGTCAACATGAGCGACAGCGCCCT | |
| | QQIFNTSYNQTYE | TGACTGCGTCCGCGACGAGGCCATCAACAAGCTCCAG | |
| | KYGNVSVFETTGG | CAGATCTTCAACACCTCCTACAACCAGACTTATGAGAA | |
| | LVVFWQGIKQKSL | GTACGGAAACGTCTCCGTTTTCGAGACAACAGGAGGC | |
| | VELERLANRSSLN | CTGGTTGTCTTCTGGCAGGGCATTAAGCAGAAGTCCCT | |
| | ITHNRTKRSTDGN | CGTCGAGCTGGAGAGACTCGCCAACCGCTCCTCCCTCA | |
| | NATHLSNMESVH | ACCTCACCCACAACCGCACCAAGCGCTCCACCGACGG | |
| | NLVYAQLQFTYDT | CAACAATGCTACACACCTGAGCAACATGGAGTCCGTC | |
| | LRGYINRALAQIA | CACAACCTCGTCTACGCCCAGCTCCAGTTCACCTACGA | |
| | EAWCVDQRRTLE | CACCCTCCGCGGCTACATCAACCGCGCCCTCGCCCAGA | |
| | VFKELSKINPSAIL | TCGCCGAGGCGTGGTGCGTCGACCAGCGCCGCACCCT | |
| | SAIYNKPIAARFM | CGAGGTCTTCAAGGAGCTGTCCAAGATCAACCCTAGC | |
| | GDVLGLASCVTIN | GCCATCCTGTCCGCAATCTATAACAAGCCTATCGCGGC | |
| | QTSVKVLRDMNV | TAGGTTCATGGGCGATGTGCTCGGCCTCGCCTCCTGCG | |
| | KESPGRCYSRPVVI | TGACTATTAATCAGACCAGCGTCAAGGTGCTGCGCGA | |
| | FNFANSSYVQYGQ | CATGAACGTGAAGGAGAGCCCTGGCCGCTGCTATTCC | |
| | LGEDNEILLGNHR | AGGCCCGTCGTCATCTTCAATTTCGCCAATTCCAGCTA | |
| | TEECQLPSLKIFIA | TGTCCAGTACGGCCAGCTCGGCGAGGACAACGAGATC | |
| | GNSAYEYVDYLF | CTGCTTGGCAACCACCGCACCGAGGAGTGTCAGCTCC | |
| | KRMIDLSSISTVDS | CTAGCCTGAAGATTTTCATTGCCGGCAATAGCGCTTAT | |
| | MIALDIDPLENTDF | GAGTATGTGGACTACCTCTTCAAGCGCATGATCGACCT | |
| | RVLELYSQKELRS | CTCCTCCATCTCCACCGTCGACTCCATGATCGCCCTGG | |
| | SNVFDLEEIMREF | ACATCGACCCACTGGAGAACACCGACTTCCGCGTGCT | |
| | NSYKQHHHHHH | CGAACTCTACTCCCAGAAGGAACTGAGATCAAGCAAC | |
| | (SEQ ID NO: 83) | GTGTTCGACCTCGAGGAGATCATGCGCGAGTTCAACTC | |
| | | TTATAAGCAGCACCACCACCATCATCAC (SEQ ID NO: | |
| | | 132) | |
| SE_CMV_ | MLRLLLRHHFHCL | ATGCTCAGACTTCTCCTCAGACACCACTTCCACTGCCT | C2/ |
| UL130_021 | LLCAVWATPCLAS | CTTGCTTTGTGCCGTCTGGGCCACACCTTGCCTCGCCA | CAP1/ |
| | PWSTLTANQNPSP | GCCCTTGGAGCACCTTGACAGCCAACCAGAACCCTTCC | T100 |
| | PWSKLTYSKPHDA | CCTCCTTGGTCAAAGTTGACCTACAGCAAGCCTCACGA | |
| | ATFYCPFLYPSPPR | CGCTGCTACCTTCTACTGTCCATTCCTGTACCCTAGCCC | |
| | SPLQFSGFQRVST | TCCAAGATCTCCGCTGCAGTTCAGCGGCTTCCAGAGGG | |
| | GPECRNETLYLLY | TGTCTACCGGACCTGAGTGCAGGAATGAGACGCTGTA | |
| | NREGQTLVERSST | CCTGCTGTACAACAGAGAGGGCCAGACCCTGGTGGAA | |
| | WVKKVIWYLSGR | AGAAGCTCCACCTGGGTCAAGAAGGTAATCTGGTACC | |
| | NQTILQRMPRTAS | TGAGCGGCAGAAACCAGACAATACTCCAGAGAATGCC | |
| | KPSDGNVQISVED | ACGGACCGCTAGCAAGCCTAGCGATGGAAACGTGCAG | |
| | AKIFGAHMVPKQT | ATTAGCGTGGAGGACGCAAAGATTTTCGGCGCCCACA | |
| | KLLRFVVNDGTRY | TGGTGCCAAAGCAGACAAAGCTGCTGCGGTTCGTGGT | |
| | QMCVMKLESWAH | CAACGACGGCACCCGGTACCAGATGTGCGTGATGAAG | |
| | VFRDYSVSFQVRL | CTGGAGAGCTGGGCTCACGTGTTCAGAGATTACTCTGT | |
| | TFTEANNQTYTFC | GAGCTTCCAAGTGCGGCTCACCTTCACCGAAGCCAAC | |
| | THPNLIV (SEQ ID | AATCAGACCTACACTTTCTGTACTCACCCTAACCTGAT | |
| | NO: 65) | CGTG (SEQ ID NO: 133) | |
| SE_CMV_ | MLRLLLRHHFHCL | ATGCTTAGACTCCTCCTCAGACACCACTTCCATTGTCT | C2/ |
| UL130_022 | LLCAVWATPCLAS | CCTTTTGTGCGCCGTGTGGGCCACCCCTTGCCTTGCAT | CAP1/ |
| | PWSTLTANQNPSP | CACCTTGGTCTACCCTCACCGCCAACCAGAACCCTAGC | T100 |
| | PWSKLTYSKPHDA | CCTCCTTGGAGTAAGTTAACATACTCTAAGCCGCACGA | |
| | ATFYCPFLYPSPPR | CGCCGCCACCTTCTACTGTCCTTTCCTCTACCCAAGCC | |
| | SPLQFSGFQRVST | CACCTCGTAGCCCACTTCAGTTCTCTGGATTCCAGAGA | |
| | GPECRNETLYLLY | GTTTCAACAGGCCCTGAGTGTCGGAACGAGACTCTGT | |
| | NREGQTLVERSST | ACCTGTTGTATAACAGAGAGGGACAGACCCTGGTGGA | |
| | WVKKVIWYLSGR | GCGGTCCTCCACCTGGGTGAAGAAGGTGATCTGGTAT | |
| | NQTILQRMPRTAS | CTGAGCGGCAGAAACCAGACCATCCTGCAGCGGATGC | |
| | KPSDGNVQISVED | CAAGGACCGCTAGCAAGCCAAGCGACGGCAATGTGCA | |
| | AKIFGAHMVPKQT | GATTAGCGTGGAGGATGCTAAGATTTTCGGCGCACAC | |
| | KLLRFVVNDGTRY | ATGGTTCCTAAGCAGACCAAGCTGTTACGGTTCGTGGT | |
| | QMCVMKLESWAH | GAACGATGGAACTCGGTACCAAATGTGCGTGATGAAG | |
| | VFRDYSVSFQVRL | CTGGAGTCATGGGCACATGTGTTCCGTGACTACTCTGT | |
| | TFTEANNQTYTFC | TTCTTTCCAGGTGCGCCTGACCTTCACCGAGGCCAATA | |
| | THPNLIV (SEQ ID | ACCAGACATACACCTTCTGTACGCACCCAAATCTGATC | |
| | NO: 65) | GTA (SEQ ID NO: 134) | |
| SE_CMV_ | MLRLLLRHHFHCL | ATGCTCAGACTTTTGCTCAGACACCACTTCCACTGTTT | C2/ |
| UL130_023 | LLCAVWATPCLAS | GTTGTTATGTGCCGTGTGGGCTACCCCTTGCCTCGCAT | CAP1/ |
| | PWSTLTANQNPSP | CTCCGTGGTCCACACTCACAGCCAACCAGAATCCTTCT | T100 |
| | PWSKLTYSKPHDA | CCTCCTTGGAGCAAGCTCACATATAGCAAGCCTCACG | |
| | ATFYCPFLYPSPPR | ACGCGGCAACCTTCTACTGCCCATTCCTGTATCCTTCT | |
| | SPLQFSGFQRVST | CCTCCGCGGAGCCCTCTGCAGTTCTCCGGATTCCAGAG | |
| | GPECRNETLYLLY | AGTGTCCACCGGTCCTGAGTGCAGAAATGAAACACTG | |
| | NREGQTLVERSST | TATCTTCTCTACAACAGAGAGGGCCAGACCCTTGTGGA | |
| | WVKKVIWYLSGR | GAGAAGCAGCACCTGGGTGAAGAAGGTCATTTGGTAT | |
| | NQTILQRMPRTAS | CTGTCTGGCAGAAACCAGACCATACTGCAGCGGATGC | |
| | KPSDGNVQISVED | CAAGAACAGCCTCCAAGCCATCCGACGGTAACGTGCA | |
| | AKIFGAHMVPKQT | GATCTCCGTGGAGGACGCCAAGATTTTCGGCGCCCAC | |
| | KLLRFVVNDGTRY | ATGGTGCCAAAGCAGACCAAGCTGCTGAGATTCGTGG | |
| | QMCVMKLESWAH | TGAACGATGGCACCAGGTACCAGATGTGCGTTATGAA | |
| | VFRDYSVSFQVRL | GCTTGAGTCCTGGGCTCACGTGTTCAGAGACTACTCTG | |
| | TFTEANNQTYTFC | TGAGCTTCCAGGTGAGACTGACATTCACAGAGGCCAA | |
| | THPNLIV (SEQ ID | CAACCAGACTTACACCTTCTGCACGCATCCTAATCTGA | |
| | NO: 65) | TCGTG (SEQ ID NO: 135) | |
| SE_CMV_ | MLRLLLRHHFHCL | ATGCTCAGACTTCTTCTCAGACACCACTTCCACTGTTT | C2/ |
| UL130_024 | LLCAVWATPCLAS | GCTTCTCTGCGCAGTGTGGGCAACCCCTTGCCTTGCTT | CAP1/ |
| | PWSTLTANQNPSP | CCCCTTGGTCGACTCTCACCGCCAACCAGAATCCAAGC | T100 |
| | PWSKLTYSKPHDA | CCTCCTTGGAGCAAGCTCACTTACAGCAAGCCGCACG | |
| | ATFYCPFLYPSPPR | ACGCCGCCACCTTCTACTGTCCTTTCCTGTACCCTAGC | |
| | SPLQFSGFQRVST | CCTCCAAGATCTCCTCTGCAATTCTCTGGATTCCAGAG | |
| | GPECRNETLYLLY | AGTGAGCACCGGCCCAGAGTGCCGGAACGAGACTCTG | |
| | NREGQTLVERSST | TATCTGCTGTACAATAGGGAGGGACAAACCCTGGTGG | |
| | WVKKVIWYLSGR | AGAGGAGCAGCACATGGGTGAAGAAGGTGATCTGGTA | |
| | NQTILQRMPRTAS | CCTGAGCGGCAGAAACCAGACCATCCTGCAGAGAATG | |
| | KPSDGNVQISVED | CCACGGACCGCCAGCAAGCCAAGCGATGGCAACGTCC | |
| | AKIFGAHMVPKQT | AGATTAGCGTGGAAGACGCCAAGATCTTCGGAGCCCA | |
| | KLLRFVVNDGTRY | CATGGTGCCTAAGCAGACCAAGCTTCTGCGATTCGTGG | |
| | QMCVMKLESWAH | TGAACGACGGTACCCGCTACCAAATGTGCGTGATGAA | |
| | VFRDYSVSFQVRL | GCTGGAGTCATGGGCCCACGTCTTCCGCGACTACAGC | |
| | TFTEANNQTYTFC | GTATCCTTCCAGGTGAGGCTTACCTTCACCGAGGCCAA | |
| | THPNLIV (SEQ ID | CAACCAAACCTACACATTCTGCACCCATCCAAATTTGA | |
| | NO: 65) | TTGTG (SEQ ID NO: 136) | |
| SE_CMV_ | MLRLLLRHHFHCL | ATGCTCAGACTATTGTTGAGACATCACTTCCATTGCCT | C2/ |
| UL130_025 | LLCAVWATPCLAS | CCTTTTGTGCGCCGTGTGGGCTACCCCTTGCCTCGCCT | CAP1/ |
| | PWSTLTANQNPSP | CACCTTGGAGCACCTTGACCGCCAACCAGAACCCGAG | T100 |
| | PWSKLTYSKPHDA | CCCTCCGTGGTCAAAGCTCACCTACAGCAAGCCTCACG | |
| | ATFYCPFLYPSPPR | ACGCCGCAACCTTCTATTGTCCATTCCTGTACCCTTCTC | |
| | SPLQFSGFQRVST | CGCCGAGGTCCCCTCTTCAGTTCAGCGGATTCCAGAGA | |
| | GPECRNETLYLLY | GTGTCTACCGGACCAGAATGCAGAAACGAAACACTGT | |
| | NREGQTLVERSST | ATCTGCTGTACAACCGGGAGGGCCAGACCCTGGTCGA | |
| | WVKKVIWYLSGR | GCGGAGCTCTACCTGGGTCAAGAAGGTTATATGGTAT | |
| | NQTILQRMPRTAS | CTGAGCGGCAGGAACCAGACCATCCTGCAGCGCATGC | |
| | KPSDGNVQISVED | CTAGAACCGCTAGCAAGCCAAGCGACGGCAACGTTCA | |
| | AKIFGAHMVPKQT | GATCTCCGTGGAGGACGCTAAGATCTTCGGCGCCCAT | |
| | KLLRFVVNDGTRY | ATGGTGCCAAAGCAGACTAAGCTGCTGAGATTCGTGG | |
| | QMCVMKLESWAH | TAAACGACGGCACAAGATATCAGATGTGCGTGATGAA | |
| | VFRDYSVSFQVRL | GCTGGAGAGCTGGGCTCATGTGTTCAGGGACTACTCC | |
| | TFTEANNQTYTFC | GTGAGTTTCCAGGTGAGGCTGACATTCACCGAGGCTA | |
| | THPNLIV (SEQ ID | ATAATCAGACCTACACCTTCTGCACTCACCCAAATCTG | |
| | NO: 65) | ATCGTG (SEQ ID NO: 137) | |
| SE_CMV_ | MLRLLLRHHFHCL | ATGCTCAGATTATTGCTCAGACACCACTTCCACTGCCT | C2/ |
| UL130_026 | LLCAVWATPCLAS | CCTCTTGTGCGCCGTGTGGGCGACACCGTGTCTCGCAA | CAP1/ |
| | PWSTLTANQNPSP | GCCCTTGGTCCACACTAACGGCCAACCAGAACCCTAG | TI00 |
| | PWSKLTYSKPHDA | CCCTCCTTGGAGCAAGCTCACTTATAGCAAGCCACACG | |
| | ATFYCPFLYPSPPR | ATGCGGCCACTTTCTACTGTCCTTTCCTGTATCCATCCC | |
| | SPLQFSGFQRVST | CTCCTAGATCTCCTCTGCAGTTCAGCGGATTCCAGAGA | |
| | GPECRNETLYLLY | GTATCTACTGGCCCTGAGTGCAGAAATGAAACCCTCTA | |
| | NREGQTLVERSST | TCTCCTGTACAATCGGGAGGGCCAGACTTTGGTGGAG | |
| | WVKKVIWYLSGR | CGCAGCTCCACCTGGGTGAAGAAGGTGATCTGGTACC | |
| | NQTILQRMPRTAS | TGAGCGGCAGAAACCAGACCATCCTACAGAGGATGCC | |
| | KPSDGNVQISVED | AAGGACCGCCAGCAAGCCATCTGACGGCAACGTGCAG | |
| | AKIFGAHMVPKQT | ATCTCTGTGGAGGACGCCAAGATCTTCGGAGCCCATAT | |
| | KLLRFVVNDGTRY | GGTGCCTAAGCAGACAAAGCTGTTGAGGTTCGTCGTG | |
| | QMCVMKLESWAH | AATGACGGCACAAGATACCAGATGTGTGTGATGAAGC | |
| | VFRDYSVSFQVRL | TGGAGAGCTGGGCTCACGTGTTCCGAGACTACAGCGT | |
| | TFTEANNQTYTFC | CTCGTTCCAGGTGAGACTGACATTCACCGAGGCAAAC | |
| | THPNLIV (SEQ ID | AACCAGACCTACACCTTCTGTACGCACCCTAACCTGAT | |
| | NO: 65) | CGTT (SEQ ID NO: 138) | |
| SE_CMV_ | MLRLLLRHHFHCL | ATGCTTCGGCTCCTCCTTCGGCACCACTTCCACTGCCT | C2/ |
| UL130_027 | LLCAVWATPCLAS | CCTCCTCTGCGCCGTGTGGGCCACACCTTGCCTCGCCA | CAP1/ |
| | PWSTLTANQNPSP | GCCCCTGGAGCACCCTCACCGCCAACCAGAACCCCAG | T100 |
| | PWSKLTYSKPHDA | CCCTCCGTGGTCTAAGTTAACCTACAGCAAGCCCCACG | |
| | ATFYCPFLYPSPPR | ACGCCGCCACCTTCTACTGCCCCTTCCTGTACCCTTCA | |
| | SPLQFSGFQRVST | CCGCCGCGGAGCCCGCTGCAGTTCAGCGGCTTCCAGC | |
| | GPECRNETLYLLY | GGGTGAGCACCGGCCCCGAGTGCCGGAACGAGACGCT | |
| | NREGQTLVERSST | GTACCTGCTGTACAACCGGGAGGGCCAGACCCTGGTG | |
| | WVKKVIWYLSGR | GAGCGGAGCAGCACCTGGGTGAAGAAGGTGATCTGGT | |
| | NQTILQRMPRTAS | ACCTGAGCGGCCGGAACCAGACCATCCTGCAGCGGAT | |
| | KPSDGNVQISVED | GCCCCGGACCGCCTCAAAGCCAAGCGACGGCAACGTG | |
| | AKIFGAHMVPKQT | CAGATCAGCGTGGAGGACGCCAAGATCTTCGGCGCCC | |
| | KLLRFVVNDGTRY | ACATGGTGCCCAAGCAGACCAAGTTGCTGCGCTTCGT | |
| | QMCVMKLESWAH | GGTGAACGACGGCACCCGGTACCAGATGTGCGTGATG | |
| | VFRDYSVSFQVRL | AAGCTGGAGAGCTGGGCCCACGTGTTCCGGGACTACA | |
| | TFTEANNQTYTFC | GCGTGAGCTTCCAGGTGCGGCTGACATTCACCGAGGC | |
| | THPNLIV (SEQ ID | CAACAATCAGACCTACACCTTCTGCACCCACCCCAACC | |
| | NO: 65) | TGATCGTG (SEQ ID NO: 139) | |
| SE_CMV_ | MLRLLLRHHFHCL | ATGTTACGGCTCCTTCTCCGCCACCACTTCCACTGCCT | C2/ |
| UL130_028 | LLCAVWATPCLAS | CTTACTCTGCGCCGTGTGGGCCACTCCATGCCTTGCCA | CAP1/ |
| | PWSTLTANQNPSP | GCCCCTGGAGCACCTTGACCGCCAACCAGAACCCCAG | T100 |
| | PWSKLTYSKPHDA | CCCACCTTGGAGTAAGCTCACCTACAGCAAGCCCCAC | |
| | ATFYCPFLYPSPPR | GACGCCGCCACCTTCTACTGCCCCTTCCTGTATCCGAG | |
| | SPLQFSGFQRVST | CCCACCGCGGAGCCCGCTGCAGTTCAGCGGCTTCCAG | |
| | GPECRNETLYLLY | CGGGTGAGCACCGGCCCCGAGTGCCGGAACGAAACCC | |
| | NREGQTLVERSST | TGTACCTGCTGTACAACCGGGAGGGCCAGACCCTGGT | |
| | WVKKVIWYLSGR | GGAGCGGAGCAGCACCTGGGTGAAGAAGGTGATCTGG | |
| | NQTILQRMPRTAS | TACCTGAGCGGCCGGAACCAGACCATCCTGCAGCGGA | |
| | KPSDGNVQISVED | TGCCCCGGACCGCTAGTAAGCCTAGCGACGGCAACGT | |
| | AKIFGAHMVPKQT | GCAGATCAGCGTGGAGGACGCCAAGATCTTCGGCGCC | |
| | KLLRFVVNDGTRY | CACATGGTGCCCAAGCAGACCAAGCTGCTTAGGTTCG | |
| | QMCVMKLESWAH | TGGTGAACGACGGCACCCGGTACCAGATGTGCGTGAT | |
| | VFRDYSVSFQVRL | GAAGCTGGAGAGCTGGGCCCACGTGTTCCGGGACTAC | |
| | TFTEANNQTYTFC | AGCGTGAGCTTCCAGGTGCGGCTGACCTTCACCGAGG | |
| | THPNLIV (SEQ ID | CCAACAACCAGACATACACCTTCTGCACCCACCCCAA | |
| | NO: 65) | CCTGATCGTG (SEQ ID NO: 140) | |
| SE_CMV_ | MLRLLLRHHFHCL | ATGCTACGGCTCCTACTCCGCCACCACTTCCACTGCTT | C2/ |
| UL130_029 | LLCAVWATPCLAS | ACTTTTGTGCGCCGTGTGGGCCACACCATGCTTGGCCA | CAP1/ |
| | PWSTLTANQNPSP | GCCCCTGGAGCACCCTCACCGCCAACCAGAACCCCTC | T100 |
| | PWSKLTYSKPHDA | ACCTCCCTGGTCCAAGCTCACCTACTCCAAGCCCCACG | |
| | ATFYCPFLYPSPPR | ACGCCGCCACCTTCTACTGCCCCTTCCTCTATCCATCTC | |
| | SPLQFSGFQRVST | CTCCACGCAGCCCACTCCAGTTCTCCGGCTTCCAGCGC | |
| | GPECRNETLYLLY | GTCTCCACCGGCCCCGAGTGCCGCAACGAGACGCTCT | |
| | NREGQTLVERSST | ACCTCCTCTACAACCGCGAGGGCCAGACCCTCGTCGA | |
| | WVKKVIWYLSGR | GAGGTCATCCACCTGGGTCAAGAAGGTCATCTGGTAC | |
| | NQTILQRMPRTAS | CTCTCCGGCCGCAACCAGACCATCCTCCAGCGCATGCC | |
| | KPSDGNVQISVED | CCGCACCGCGTCTAAGCCGTCCGACGGCAACGTCCAG | |
| | AKIFGAHMVPKQT | ATCTCCGTCGAGGACGCCAAGATCTTCGGCGCCCACAT | |
| | KLLRFVVNDGTRY | GGTCCCCAAGCAGACCAAGCTCCTCCGCTTCGTCGTCA | |
| | QMCVMKLESWAH | ACGACGGCACCCGCTACCAGATGTGCGTCATGAAGCT | |
| | VFRDYSVSFQVRL | CGAGTCCTGGGCCCACGTCTTCCGCGACTACTCCGTCT | |
| | TFTEANNQTYTFC | CCTTCCAGGTCCGCCTCACCTTCACCGAGGCCAATAAC | |
| | THPNLIV (SEQ ID | CAGACTTACACCTTCTGCACCCACCCCAACCTCATCGT | |
| | NO: 65) | C (SEQ ID NO: 141) | |
| SE_CMV_ | MLRLLLRHHFHCL | ATGCTACGGCTCCTACTCCGCCACCACTTCCACTGCTT | C2/ |
| UL130_029 | LLCAVWATPCLAS | ACTTTGTGCGCCGTGTGGGCCACACCATGCTTGGCCA | CAP1/ |
| | PWSTLTANQNPSP | GCCCCTGGAGCACCCTCACCGCCAACCAGAACCCCTC | T100 |
| | PWSKLTYSKPHDA | ACCTCCCTGGTCCAAGCTCACCTACTCCAAGCCCCACG | |
| | ATFYCPFLYPSPPR | ACGCCGCCACCTTCTACTGCCCCTTCCTCTATCCATCTC | |
| | SPLQFSGFQRVST | CTCCACGCAGCCCACTCCAGTTCTCCGGCTTCCAGCGC | |
| | GPECRNETLYLLY | GTCTCCACCGGCCCCGAGTGCCGCAACGAGACGCTCT | |
| | NREGQTLVERSST | ACCTCCTCTACAACCGCGAGGGCCAGACCCTCGTCGA | |
| | WVKKVIWYLSGR | GAGGTCATCCACCTGGGTCAAGAAGGTCATCTGGTAC | |
| | NQTILQRMPRTAS | CTCTCCGGCCGCAACCAGACCATCCTCCAGCGCATGCC | |
| | KPSDGNVQISVED | CCGCACCGCGTCTAAGCCGTCCGACGGCAACGTCCAG | |
| | AKIFGAHMVPKQT | ATCTCCGTCGAGGACGCCAAGATCTTCGGCGCCCACAT | |
| | KLLRFVVNDGTRY | GGTCCCCAAGCAGACCAAGCTCCTCCGCTTCGTCGTCA | |
| | QMCVMKLESWAH | ACGACGGCACCCGCTACCAGATGTGCGTCATGAAGCT | |
| | VFRDYSVSFQVRL | CGAGTCCTGGGCCCACGTCTTCCGCGACTACTCCGTCT | |
| | TFTEANNQTYTFC | CCTTCCAGGTCCGCCTCACCTTCACCGAGGCCAATAAC | |
| | THPNLIV (SEQ ID | CAGACTTACACCTTCTGCACCCACCCCAACCTCATCGT | |
| | NO: 65) | C (SEQ ID NO: 142) | |
| SE_CMV_ | MLRLLLRHHFHCL | ATGCTTCGGCTCCTCCTAAGGCACCACTTCCACTGCCT | C2/ |
| UL130_030 | LLCAVWATPCLAS | TTTGCTTTGCGCCGTGTGGGCCACCCCTTGCTTGGCCA | CAP1/ |
| | PWSTLTANQNPSP | GCCCCTGGAGCACCCTCACCGCCAACCAGAACCCCTC | T100 |
| | PWSKLTYSKPHDA | CCCTCCCTGGTCCAAGCTCACCTACTCCAAGCCCCACG | |
| | ATFYCPFLYPSPPR | ACGCCGCCACCTTCTACTGCCCCTTCCTCTACCCGTCC | |
| | SPLQFSGFQRVST | CCTCCACGCAGCCCACTCCAGTTCTCCGGCTTCCAGCG | |
| | GPECRNETLYLLY | CGTCTCCACCGGCCCCGAGTGCCGCAACGAAACACTC | |
| | NREGQTLVERSST | TACCTCCTCTACAACCGCGAGGGCCAGACCCTCGTCGA | |
| | WVKKVIWYLSGR | GCGCTCCTCCACCTGGGTCAAGAAGGTCATCTGGTACC | |
| | NQTILQRMPRTAS | TCTCCGGCCGCAACCAGACCATCCTCCAGCGCATGCCC | |
| | KPSDGNVQISVED | CGCACCGCAAGCAAGCCATCCGACGGCAACGTCCAGA | |
| | AKIFGAHMVPKQT | TCTCCGTCGAGGACGCCAAGATCTTCGGCGCCCACATG | |
| | KLLRFVVNDGTRY | GTCCCCAAGCAGACCAAGCTCCTCCGCTTCGTCGTCAA | |
| | QMCVMKLESWAH | CGACGGCACCCGCTACCAGATGTGCGTCATGAAGCTC | |
| | VFRDYSVSFQVRL | GAGTCCTGGGCCCACGTCTTCCGCGACTACTCCGTCTC | |
| | TFTEANNQTYTFC | CTTCCAGGTCCGCCTCACCTTCACCGAGGCCAATAATC | |
| | THPNLIV (SEQ ID | AGACATACACCTTCTGCACCCACCCCAACCTCATCGTC | |
| | NO: 65) | (SEQ ID NO: 143) | |
| UL131A | MRLCRVWLSVCL | ATGCGGCTGTGTCGGGTGTGGCTGTCTGTTTGTCTGTG | C2/ |
| | CAVVLGQCQRET | CGCCGTGGTGCTGGGTCAGTGCCAGCGGGAAACCGCG | Cap1/ |
| | AEKNDYYRVPHY | GAAAAGAACGATTATTACCGAGTACCGCATTACTGGG | tailless |
| | WDACSRALPDQT | ACGCGTGCTCTCGCGCGCTGCCCGACCAAACCCGTTAC | |
| | RYKYVEQLVDLTL | AAGTATGTGGAACAGCTCGTGGACCTCACGTTGAACT | |
| | NYHYDASHGLDN | ACCACTACGATGCGAGCCACGGCTTGGACAACTTTGA | |
| | FDVLKRINVTEVS | CGTGCTCAAGAGAATCAACGTGACCGAGGTGTCGTTG | |
| | LLISDFRRQNRRG | CTCATCAGCGACTTTAGACGTCAGAACCGTCGCGGCG | |
| | GTNKRTTFNAAGS | GCACCAACAAAAGGACCACGTTCAACGCCGCCGGTTC | |
| | LAPHARSLEFSVR | GCTGGCGCCACACGCCCGGAGCCTCGAGTTCAGCGTG | |
| | LFAN (SEQ ID NO: | CGCCTCTTTGCCAAC (SEQ ID NO: 144) | |
| | 67) | | |
| UL131A | MRLCRVWLSVCL | ATGCGGCTGTGTCGGGTGTGGCTGTCTGTTTGTCTGTG | C2/ |
| | CAVVLGQCQRET | CGCCGTGGTGCTGGGTCAGTGCCAGCGGGAAACCGCG | no cap/ |
| | AEKNDYYRVPHY | GAAAAGAACGATTATTACCGAGTACCGCATTACTGGG | T100 |
| | WDACSRALPDQT | ACGCGTGCTCTCGCGCGCTGCCCGACCAAACCCGTTAC | |
| | RYKYVEQLVDLTL | AAGTATGTGGAACAGCTCGTGGACCTCACGTTGAACT | |
| | NYHYDASHGLDN | ACCACTACGATGCGAGCCACGGCTTGGACAACTTTGA | |
| | FDVLKRINVTEVS | CGTGCTCAAGAGAATCAACGTGACCGAGGTGTCGTTG | |
| | LLISDFRRQNRRG | CTCATCAGCGACTTTAGACGTCAGAACCGTCGCGGCG | |
| | GTNKRTTFNAAGS | GCACCAACAAAAGGACCACGTTCAACGCCGCCGGTTC | |
| | LAPHARSLEFSVR | GCTGGCGCCACACGCCCGGAGCCTCGAGTTCAGCGTG | |
| | LFAN (SEQ ID NO: | CGGCTCTTTGCCAAC (SEQ ID NO: 144) | |
| | 67) | | |
| UL131A | MRLCRVWLSVCL | ATGCGGCTGTGTCGGGTGTGGCTGTCTGTTTGTCTGTG | C1/ |
| | CAVVLGQCQRET | CGCCGTGGTGCTGGGTCAGTGCCAGCGGGAAACCGCG | CAP1/ |
| | AEKNDYYRVPHY | GAAAAGAACGATTATTACCGAGTACCGCATTACTGGG | T100 |
| | WDACSRALPDQT | ACGCGTGCTCTCGCGCGCTGCCCGACCAAACCCGTTAC | |
| | RYKYVEQLVDLTL | AAGTATGTGGAACAGCTCGTGGACCTCACGTTGAACT | |
| | NYHYDASHGLDN | ACCACTACGATGCGAGCCACGGCTTGGACAACTTTGA | |
| | FDVLKRINVTEVS | CGTGCTCAAGAGAATCAACGTGACCGAGGTGTCGTTG | |
| | LLISDFRRQNRRG | CTCATCAGCGACTTTAGACGTCAGAACCGTCGCGGCG | |
| | GTNKRTTFNAAGS | GCACCAACAAAAGGACCACGTTCAACGCCGCCGGTTC | |
| | LAPHARSLEFSVR | GCTGGCGCCACACGCCCGGAGCCTCGAGTTCAGCGTG | |
| | LFAN (SEQ ID NO: | CGGCTCTTTGCCAAC (SEQ ID NO: 144) | |
| | 67) | | |

*The nucleotide sequences shown in Table 13 are open reading frame sequences, which can be linked to sequences encoding a 5′UTR and a 3′UTR.
5′ UTR coding sequence:
TCAAGCTTTTGGACCCTCGTACAGAAGCTAATACGACTCACTATAGGGAAATAAGAGAGAAAAGAAGAGTAAGAAGAAATATAAGAGCCACC (SEQ ID NO: 145)
5′ UTR (without promoter) coding sequence:
GGGAAATAAGAGAGAAAAGAAGAGTAAGAAGAAATATAAGAGCCACC (SEQ ID NO: 146)
3′ UTR coding sequence:
TGATAATAGGCTGGAGCCTCGGTGGCCATGCTTCTTGCCCCTTGGGCCTCCCCCCAGCCCCTCCTCCCCTTCCTGCACCCGTACCCCCGTGGTCTTTGAATAAAGTCTGAGTGGGCGGC (SEQ ID NO: 147)
C1: No chemical modification
C2: N1-methylpseudouridine chemical modification

### Example 38 Expression of gB from Codon-Optimized mRNA Variant Constructs

HCMV glycoprotein B (gB) expression level in HEK 293 cells or on the cell surface was tested for codon-optimized gB mRNA variants (Var #1-Var #10, SEQ ID NOs: 94, 157, and 95-102, respectively, see Table 13). HEK293 cells were transiently transfected with mRNA encoding the codon-optimized gB mRNA variants using Trans IT®-mRNA Transfection Kit (Mirus Bio LLC) per the manufacturer's recommendations. At 24 hr post-transfection, cells were lysed in 1% Digitonin buffer supplemented with complete mini-EDTA free protease inhibitor cocktail tablets (ThermoFisher Scientific). Precleared lysates were resolved on Novex 4-12% Bis-Tris gels (Invitrogen) and blotted with anti-gB mouse monoclonal antibody (clone CH28, Santa Cruz Biotechnology) and mouse anti-(i actin (Cell Signaling Technology). Alexa Fluor 680 goat anti-mouse IgG (ThermoFisher Scientific) was used as secondary antibody. All images were captured on a ChemiDoc MP Imaging System (Bio-Rad Laboratories).

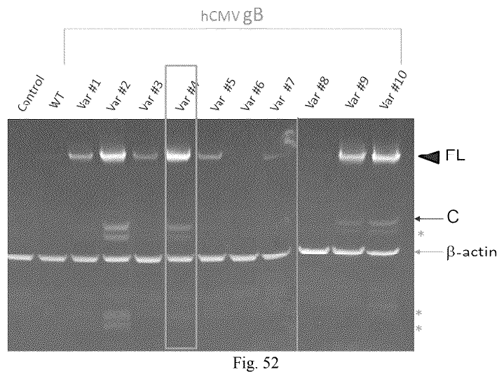

The result shows that all of the codon-optimized variants were expressed. Compared to the wild type gB mRNA, several of the codon-optimized variants (Var #1-Var #4, Var #9, and Var #10) showed enhanced expression in HEK293 cells, among which Var #4 showed the highest expression level (FIG. 52).

### EQUIVALENTS

Those skilled in the art will recognize, or be able to ascertain using no more than routine experimentation, many equivalents to the specific embodiments of the disclosure described herein. Such equivalents are intended to be encompassed by the following claims.

All references, including patent documents, disclosed herein are incorporated by reference in their entirety. This application incorporates by reference the entire contents, including all the drawings and all parts of the specification (including sequence listing or amino acid/polynucleotide sequences) of PCT Application No. PCT/US2016/058310, filed on Oct. 21, 2016, and entitled “HUMAN CYTOMEGALOVIRUS VACCINE.”

1.-180. (canceled)

181. A human cytomegalovirus (hCMV) vaccine comprising:
(a) a messenger ribonucleic acid (mRNA) polynucleotide comprising from 5′ to 3′ a 5′UTR, an open reading frame (ORF) encoding an hCMV pp65 protein, a 3′ UTR, and a poly(A) tail, wherein 100% of the uridines in the ORF of the mRNA polynucleotide are N1-methylpseudouridines; and
a lipid nanoparticle.

182. The hCMV vaccine of claim 181, wherein the hCMV pp65 protein comprises an amino acid sequence having at least 90% identity to the amino acid sequence of SEQ ID NO:71.

183. The hCMV vaccine of claim 181, wherein the hCMV pp65 protein comprises the amino acid sequence of SEQ ID NO:71.

184. The hCMV vaccine of claim 181, further comprising:
(b) an mRNA polynucleotide having from 5′ to 3′ a 5′UTR, an ORF encoding an hCMV gH protein, a 3′ UTR, and a poly(A) tail;
(c) an mRNA polynucleotide having from 5′ to 3′ a 5′UTR, an ORF encoding an hCMV gL protein, a 3′ UTR, and a poly(A) tail;
(d) an mRNA polynucleotide having from 5′ to 3′ a 5′UTR, an ORF encoding an hCMV UL128 protein, a 3′ UTR, and a poly(A) tail;
(e) an mRNA polynucleotide having from 5′ to 3′ a 5′UTR, an ORF encoding an hCMV UL130 protein, a 3′ UTR, and a poly(A) tail; and
(f) an mRNA polynucleotide having from 5′ to 3′ a 5′UTR, an ORF encoding an hCMV UL131A protein, a 3′ UTR, and a poly(A) tail;
wherein 100% of the uridines in the ORFs of each of the mRNA polynucleotides of (b) to (f) are N1-methylpseudouridines.

185. The hCMV vaccine of claim 184, wherein the hCMV pp65 protein comprises an amino acid sequence that has at least 90% identity to the amino acid sequence of SEQ ID NO:71, the hCMV gH protein comprises an amino acid sequence that has at least 90% identity to the amino acid sequence of SEQ ID NO:59, the hCMV gL protein comprises an amino acid sequence that has at least 90% identity to the amino acid sequence of SEQ ID NO:61, the hCMV UL128 protein comprises an amino acid sequence that has at least 90% identity to the amino acid sequence of SEQ ID NO:63, the hCMV UL130 protein comprises an amino acid sequence that has at least 90% identity to the amino acid sequence of SEQ ID NO:65, and the hCMV UL131 protein comprises an amino acid sequence that has at least 90% identity to the amino acid sequence of SEQ ID NO:67.

186. The hCMV vaccine of claim 184, wherein the hCMV pp65 protein comprises the amino acid sequence of SEQ ID NO:71, the hCMV gH protein comprises the amino acid sequence of SEQ ID NO:59, the hCMV gL protein comprises the amino acid sequence of SEQ ID NO:61, the hCMV UL128 protein comprises the amino acid sequence of SEQ ID NO:63, the hCMV UL130 protein comprises the amino acid sequence of SEQ ID NO:65, and the hCMV UL131A protein comprises the amino acid sequence of SEQ ID NO:67.

187. The hCMV vaccine of claim 181, wherein the lipid nanoparticle comprises an ionizable cationic lipid, a non-cationic lipid, a sterol, and a PEG-modified lipid.

188. The hCMV vaccine of claim 187, wherein the lipid nanoparticle comprises a molar ratio of 20-60% ionizable cationic lipid, 5-25% non-cationic lipid, 25-55% sterol, and 0.5-15% PEG-modified lipid.

189. A method of inducing an anti-hCMV immune response in a human subject, the method comprising administering to the subject an amount of the hCMV vaccine of claim 181 effective to produce the anti-hCMV immune response in the human subject.

190. The method of claim 189, wherein the human subject is an immunocompromised organ transplant recipient.
